(19) United States
(12) Patent Application Publication
(10) Pub. No.: US 2025/0049714 A1
(43) Pub. Date: Feb. 13, 2025

(54) SYNTHESIS OF ESTER, CARBONATE, AND CARBAMATE-DERIVED NOVEL BIODEGRADABLE IONIZABLE LIPIDS FROM METHYL RICINOLEATE OR METHYL 12-HYDROXYSTEARATE AND ITS APPLICATIONS

(71) Applicant: Massachusetts Institute of Technology, Cambridge, MA (US)

(72) Inventors: Daniel Griffith Anderson, Framingham, MA (US); Luke Hyunsik Rhym, Cambridge, MA (US); Bowen Li, Malden, MA (US); Akiva Gordon, Somerville, MA (US); Manan Rajith Singh, Arlington, MA (US); Jacob Witten, Cambridge, MA (US)

(73) Assignee: Massachusetts Institute of Technology, Cambridge, MA (US)

(21) Appl. No.: 18/717,095

(22) PCT Filed: Dec. 8, 2022
(86) PCT No.: PCT/US2022/052314

§ 371 Date: Jun. 6, 2024

Related U.S. Application Data
(60) Provisional application No. 63/287,709, filed on Dec. 9, 2021.
(60) Provisional application No. 63/314,016, filed on Feb. 25, 2022.

(51) Int. Cl. A61K 9/127; A61K 9/00; A61K 9/16; A61K 9/51; A61K 31/7105; A61K 31/711; C07C 229/12; C07C 229/16; C07D 207/06; C07D 213/38; C07D 255/02; C07D 295/13; C12N 15/88

(52) U.S. Cl. CPC A61K 9/1272; A61K 9/0075; A61K 9/1623; A61K 9/5123; A61K 31/7105; A61K 31/711; C07C 229/12; C07C 229/16; C07D 207/06; C07D 213/38; C07D 255/02; C07D 295/13; C12N 15/88

(57) ABSTRACT

Provided herein are compounds, such as compounds of Formula (I), and pharmaceutically acceptable salts, hydrates, solvates, polymorphs, co-crystals, tautomers, stereoisomers, and isotopically labeled derivatives thereof, and compositions, methods, uses, and kits thereof. The compounds provided herein are lipids useful for delivery of polynucleotides, such as mRNA, for the treatment and/or prevention of various diseases and conditions (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease).

### CROSS-REFERENCE TO RELATED APPLICATIONS

This application claims the benefit of and priority under 35 U.S.C. § 119(e) to U.S. Provisional Application No. 63/287,709, filed Dec. 9, 2021, titled SYNTHESIS OF ESTER, CARBONATE, AND CARBAMATE-DERIVED NOVEL BIODEGRADABLE IONIZABLE LIPIDS FROM METHYL RICINOLEATE OR METHYL 12-HYDROXYSTEARATE AND ITS APPLICATIONS, and to U.S. Provisional Application No. 63/314,016, filed Feb. 25, 2022, titled SYNTHESIS OF ESTER, CARBONATE, AND CARBAMATE-DERIVED NOVEL BIODEGRADABLE IONIZABLE LIPIDS FROM METHYL RICINOLEATE OR METHYL 12-HYDROXYSTEARATE AND ITS APPLICATIONS, the contents of each of which are incorporated herewith by reference in their entirety.

### STATEMENT OF SPONSORED RESEARCH

Research for this invention was supported by awards from the Cystic Fibrosis Foundation.

### BACKGROUND

Nucleic acids represent a new and promising class of therapeutics. Rather than treating conditions by dosing small molecules or proteins, DNA or RNA is administered to patients, whose cells then produce the desired pharmaceutically relevant protein from these “blueprints.” As nucleic acids are much more stable and easier to produce and modify than fully translated proteins, there is enormous potential for these drugs to treat disease, with applications ranging from cancer to chronic conditions like cystic fibrosis and even neurological disorders.¹³⁻¹⁶ Recently, nucleic acid therapeutics have come into the public eye in the form of mRNA vaccines against the SARS-CoV-2 virus.

Although these vaccines highlighted the amazing potential of nucleic acid therapeutics, they also brought to the forefront two key shortcomings: Feasibility of storage and administration of RNA lipoplexes. In the fall of 2020, even as preliminary clinical trial results offered new hope in the form of incredibly effective mRNA vaccines against COVID-19, attention immediately turned to the issue of the necessary cold storage chains across the United States and the world as a whole. A month before the Pfizer/BioNTech BNT162b2 vaccine was authorized for emergency use, hospitals were already scrambling to obtain special freezers and rural areas expressed frustration that they were unable to afford the necessary equipment to use the newly developed vaccine.¹⁷ Additionally, the feasibility of making the quality mRNA vaccines available to the developing world remains an open question due to these storage concerns.¹⁸

Although nucleic acids alone are fairly stable, mRNA therapeutics generally employ polymeric or lipid nanoparticles (LNPs) to deliver the mRNA, which, while excellent delivery carriers, rapidly aggregate in solution, losing 20% of their activity in just one week at standard refrigerator temperatures.¹¹ This aggregation unfortunately makes it infeasible for patients to store these therapeutics at home, limiting their potential for clinical translation. Concerns were also raised that vaccinating enough people could take over a year simply due to the availability of vaccination sites and personnel.¹⁹ These concerns carry over to many other applications of mRNA therapeutics, especially chronic conditions like cystic fibrosis, for which a patient-centric home-stable and self-administrable therapeutic is desirable. For cystic fibrosis in particular, which is an excellent RNA therapy candidate due to its single-gene-mutation nature, it is also desirable to have local delivery to the lungs, which cannot be trivially accomplished using conventional LNP solutions.⁹

mRNA therapeutics have significant potential to treat various diseases through protein-replacement, immunomodulation, and gene editing. Non-viral nanoparticles are promising mRNA delivery vehicles to target cells in vivo. However, effective mRNA delivery requires circumventing RNAse-mediated degradation, cellular entry, and endosomal escape. Accordingly, new compounds capable of forming delivery vehicles capable of specific mRNA delivery to tissues are needed.

### SUMMARY OF THE INVENTION

The present disclosure relates in part to new compounds (e.g., compounds of Formulae (I) and (II)) that can deliver agents to a subject or cell, and compositions and methods of using and preparing such compounds.

Provided herein are compounds (e.g., compounds of Formulae (I) and (II)), and pharmaceutically acceptable salts, solvates, tautomers, stereoisomers, and isotopically labeled derivatives thereof, and pharmaceutical compositions thereof. In certain embodiments, the compounds provided herein can form particles for delivery of various agents and can therefore be useful for the treatment and/or prevention of diseases (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease).

The present disclosure also provides methods of using the compounds and compositions provided herein, e.g., for delivering a polynucleotide to a subject or a cell. Also provided herein are methods of preparing compounds provided herein (e.g., compounds of Formulae (I) and (II)), and pharmaceutically acceptable salts, solvates, tautomers, stereoisomers, and isotopically labeled derivatives thereof. The present disclosure also provides kits comprising a compound provided herein (e.g., a compound of Formulae (I) and (II)), or a pharmaceutically acceptable salt, solvate, tautomer, stereoisomer, or isotopically labeled derivative thereof, or a pharmaceutical composition thereof.

In one aspect, the disclosure provides a compound of Formula (I):

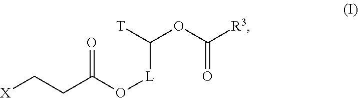

or a pharmaceutically acceptable salt, solvate, tautomer, stereoisomer, or isotopically labeled derivative thereof, wherein X, T, L, and R³ are as defined herein.

In one aspect, the compound of Formula (I) is of Formula (II):

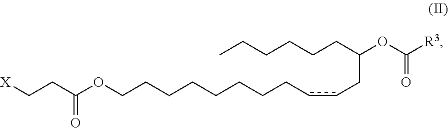

or a pharmaceutically acceptable salt, solvate, tautomer, stereoisomer, or isotopically labeled derivative thereof, wherein X,

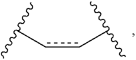

and R³ are as defined herein.

In certain embodiments, the compound of Formula (II) is of Formula (II-A):

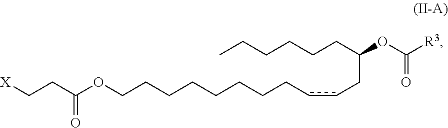

or a pharmaceutically acceptable salt, solvate, tautomer, stereoisomer, or isotopically labeled derivative thereof, wherein X,

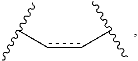

and R³ are as defined herein.

In certain embodiments, the compound of Formula (II) is of Formula (II-B):

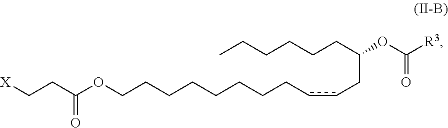

or a pharmaceutically acceptable salt, solvate, tautomer, stereoisomer, or isotopically labeled derivative thereof, wherein X,

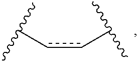

and R³ are as defined herein.

In certain embodiments, the compound of Formula (II) is of Formula (II-C):

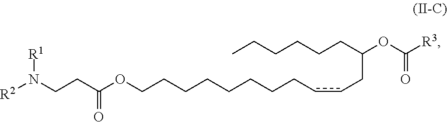

or a pharmaceutically acceptable salt, solvate, tautomer, stereoisomer, or isotopically labeled derivative thereof, wherein R¹, R²,

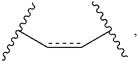

and R³ are as defined herein.

In certain embodiments, the compound of Formula (II) is of Formula (II-D):

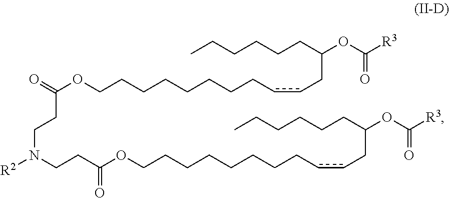

or a pharmaceutically acceptable salt, solvate, tautomer, stereoisomer, or isotopically labeled derivative thereof, wherein R¹, R²,

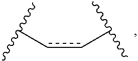

and R³ are as defined herein.

In another aspect, the disclosure provides compounds prepared by reacting a compound of Formula (III):

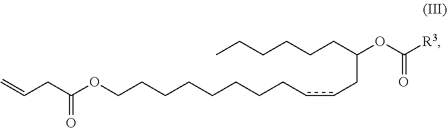

or a salt thereof, with a compound of formula:

H—X,

or a salt thereof, wherein R³,

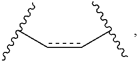

and X are as defined herein.

In another aspect, the present disclosure provides pharmaceutical compositions comprising a compound provided herein (e.g., a compound of Formula (I) or (II)), or a pharmaceutically acceptable salt, solvate, tautomer, stereoisomer, or isotopically labeled derivative thereof, and a pharmaceutically acceptable excipient. In some embodiments, the pharmaceutical composition further comprises an additional pharmaceutical agent. In certain embodiments, the additional agent is a polynucleotide. In some embodiments, the polynucleotide is mRNA.

In another aspect, the present disclosure provides inhalable dry powder compositions comprising microparticles, wherein the microparticles comprise a polynucleotide, a lipid nanoparticle, and a saccharide excipient, and wherein at least about 50% of the microparticles in the composition have an aerodynamic size of about 1 micron to about 5 microns, a physical diameter of about 3 microns to about 10 microns, or a combination thereof. In certain embodiments, the polynucleotide is encapsulated by the lipid nanoparticle. In some embodiments, the lipid nanoparticle comprises a compound provided herein (e.g., a compound of Formula (I) or (II)).

In another aspect, the present disclosure provides methods for treating and/or preventing a disease, disorder, or condition (e.g., a genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject, comprising administering to the subject a composition comprising an agent and a compound provided herein (e.g., a compound of Formula (I) or (II)), or a pharmaceutically acceptable salt, solvate, tautomer, stereoisomer, or isotopically labeled derivative thereof.

In another aspect, the present disclosure provides methods of treating or preventing a disorder or a disease by administering to a subject in need thereof a therapeutically effective amount of an inhalable dry powder composition provided herein.

In another aspect, the present disclosure provides use of an inhalable dry powder composition provided herein for the prophylaxis, treatment, and/or amelioration of a disorder or a disease.

In certain embodiments, the disease is a genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease. In certain embodiments, the disorder or disease is an allergic disease, an autoimmune disease, an infectious disease, or a cancer. In some embodiments, the disorder or disease is a lung disease, a cardiovascular disease, or a neuronal disease.

In yet another aspect, the present disclosure provides methods of delivering a polynucleotide to a subject, comprising administering to the subject a composition comprising a polynucleotide and a compound provided herein (e.g., a compound of Formula (I) or (II)), or a pharmaceutically acceptable salt, solvate, tautomer, stereoisomer, or isotopically labeled derivative thereof.

In another aspect, the present disclosure provides kits comprising a compound provided herein (e.g., a compound of Formula (I) or (II)), or a pharmaceutically acceptable salt, solvate, tautomer, stereoisomer, or isotopically labeled derivative thereof; or a pharmaceutical composition thereof, and instructions for using the compound, or pharmaceutically acceptable salt, solvate, tautomer, stereoisomer, or isotopically labeled derivative thereof, or pharmaceutical composition (e.g., for treating and/or preventing a disease or condition in a subject or delivering a polynucleotide to a subject).

In another aspect, the disclosure provides methods of preparing a compound of Formula (II), the method comprising reacting a compound of Formula (III):

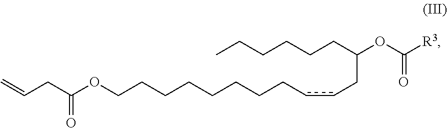

or a salt thereof, with a compound of formula:
or a salt thereof, wherein R³,

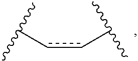

and X are as defined herein.

In another aspect, the disclosure provides methods of preparing a dry powder composition, the method comprising: a) providing an aqueous mixture comprising a polynucleotide, a lipid nanoparticle, and a saccharide excipient, wherein the polynucleotide is encapsulated by the lipid nanoparticles; and b) spray freeze drying the mixture to form microparticles, wherein at least about 50% of the microparticles in the composition have an aerodynamic size of about 1 micron to about 5 microns, a physical diameter of about 3 microns to about 10 microns, or a combination thereof, thereby preparing the dry powder composition. In some embodiments, the lipid nanoparticle comprises a compound provided herein (e.g., a compound of Formula (I) or (II)).

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description, provide non-limiting examples of the invention.

FIG. 1 shows the synthesis of the ricinoleic acrylate/12-hydroxy stearyl acrylate precursor.

FIG. 2 shows possible transformations of the ricinoleic acrylate/12-hydroxy stearyl acrylate precursor to biodegradable esters, carbonates, and carbamates.

FIG. 3 shows the synthesis of ester-, carbonate-, and carbamate-derived lipids with one tail or two tails obtained by reacting the ricinoleic acrylate/12-hydroxy stearyl acrylate precursor with primary or secondary amines, respectively.

FIGS. 4A-4C depict functional mRNA delivery using ionizable lipids. FIG. 4A shows potent and selective mRNA delivery and FLuc expression upon administration of MR-1-178-4 (left image) and MR-1-178-5 (right image) lipid nanoparticles containing FLuc mRNA.

FIG. 4B Human erythropoietin (hEPO) protein secretion upon administration of 1-178-4 (MR-1-178-4), 2-93-3 (MR-2-93-3), and MC3 (DLin-MC3-DMA) lipid nanoparticles containing hEPO mRNA. FIG. 4C shows the structures of top-performing ester-derived novel ionizable lipids MR-1-178-4, MR-1-178-5, and MR-2-93-3.

FIGS. 5A-5C depict ionizable lipid screening in ALI cultures. FIG. 5A shows luciferase mRNA delivery to ALI cultures. FIG. 5B depicts structures of hits, 177-2 (MR-1-177-2), 172-3 (MR-1-172-3), 183-2 (MR-1-183-2), and 2-90-4 (MR-2-90-4). FIG. 5C shows a confirmatory screen in primary human bronchial epithelial ALI cultures derived from both large and small airways.

FIGS. 6A and 6B show nebulized mRNA delivery with 177-2 (MR-1-177-2) and 183-2 (MR-1-183-2) (500 μg mRNA dose in a whole-body nebulization chamber; only a small fraction of the 500 μg dose is inhaled). FIG. 6A shows a comparison of 177-2 (MR-1-177-2) and 183-2 (MR-1-183-2) nebulized delivery with C12-200 nebulized delivery. FIG. 6B shows representative luminescence images of lungs following nebulization.

FIG. 7 depicts the synthesis of carbonate-derived biodegradable lipids from ricinoleic/stearyl acrylate and shows representative examples of alcohols used therein.

FIG. 8 depicts in vitro results using HEK293T cells for the 28-carbonate-derived biodegradable ionizable lipids (RCB) as compared to MD1 and PBS. Ten of the carbonate-derived ionizable lipids showed better transfection than MD-1.

FIGS. 9A-9D show in vivo and in vitro delivery using carbonate-derived biodegradable ionizable lipids. FIG. 9A shows Ivis images and quantification of the Luciferase expression of the best performing lipids (2-72-1 (RCB-2-72-1), 2-209-2 (RCB-2-209-2), 2-223-2 (RCB-2-223-2), and 2-223-3 (RCB-01-223-3)) from the first set of 28 lipids.

FIG. 9B depicts a comparison of luminescence obtain in vitro with A459 cells, in vivo with HEK cells, and in vivo by IM injection using carbonate-derived biodegradable ionizable lipids (RCB). FIG. 9C shows ROI over MD-1 through intratracheal administration for the top performing lipids (72-1 (RCB-02-72-1), 78-3 (RCB-02-78-3), 209-2 (RCB-2-209-2), 79-3 (RCB-02-79-3), 76-1 (RCB-02-76-1), 76-3 (RCB-02-76-3), 223-3 (RCB-01-223-3)). FIG. 9D shows dose response for MD-1, 223-3 (RCB-01-223-3), and 76-3 (RCB-02-76-3) using 5 μg, 2.5 μg, 1.5 μg, or 1 μg of mRNA.

FIGS. 10A and 10B show amine groups and lipid tails used to generate a structural library. FIG. 10A shows amines. FIG. 10B shows carbonate lipid tails.

FIGS. 11A-11C shows screening and luciferase expression of carbonate lipids. FIG. 11A shows a heat map depicting the relative luciferase expression/cell viability after incubating A549 cells with mLuc-loaded LNPs overnight. FIG. 11B shows IVIS images of luciferase expression of the best performing lipids (MID1, 102-5 (RCB-02-102-5), 19-8 (RCB-19-8; also shown herein as RCB-02-113-2), 104-1 (RCB-02-104-1), 104-4 (RCB-02-104-4), 4-8 (RCB-2-4-8), 76-3 (RCB-02-76-3), 223-3 (RCB-01-223-3)) from the combinatorial library in the lungs of mice after I.T. administration (0.125 mg/kg mRNA). FIG. 11C shows structures of top performing lipids.

FIG. 12A depicts a quantitative assessment of lung cells transfected by RCB-2-4-8 incorporating LNPs determined using a Cre-loxP mouse model designed to express tdTomato only in cells that translate cre-recombinase mRNA. Lung cells expressing tdTomato fluorescence were analyzed by flow cytometry. FIG. 12B depicts the percentage of untreated, NP-Cre X1, AAV+NP-Cre X1, and NP-Cre X3 cells expressing tdTomato fluorescence.

FIG. 12C shows quantification of edited (tdTomatao+) club cells (left) and ciliated cells (right).

FIGS. 13A and 13B depict detection of genome editing in mouse lung. FIG. 13A shows lung cells from untreated Ai9 mice. FIG. 13B shows tdTomato+ cells in the airway after I.T. administration of RCB-2-4-8 mCas9/sgRNA LNPs, indicating genome editing. FIG. 13C shows representative native fluorescence images of lung sections. FIG. 13D shows quantification of tdTomato+ cells. LNPhigh-SPCas9mRNA serves as a negative control. Scale bar: 100 Um. N=9 sections from 3 mice. Errors bars are S.D.

FIGS. 14A-14C show SEM images of dry powders prepared with three proprietary lipids, MD-1 (FIG. 14A), RCB-01-223-3 (FIG. 14B), and RCB-02-76-3 (FIG. 14C), showing irregular particle shape with approximate size between 1 and 10 microns. FIG. 14D depicts MMAD data showed aerodynamic size with approximately 70% of observed powder falling into the ideal 1-5-micron range for RCB-01-223-3. FIG. 14E shows transfection distribution in mouse lung following dry powder administration using proprietary lipid RCB-02-76-3 via tracheal administration; all lobes show significant and comparable luminescence.

FIG. 15 shows dosing of dry powders comprising lipids MD-1, 223-3 (RCB-01-223-3), or 76-3 (RCB-02-76-3) to A549 and Hela cells.

FIGS. 16A-16B show mannitol formulation conditions through formulation studies in in vitro transfection of Hela cells. FIG. 16A compares transfection of several cryoprotectants and concentrations tested (M=mannitol, L=lactose, T=trehalose). Percentages indicate w/v proportion at nanoparticle synthesis. FIG. 16B compares transfection of the addition of mannitol before and after formulation showing that although mannitol disrupts formulation significantly, it leads to significantly stabler particles for a more-active final powder.

FIG. 17 depicts transfection distribution in rat lung following administration of powder using commercial lipid D-Lin-MC3-DMA. Despite tracheal administration, luminescence is significant throughout the lungs.

FIG. 18 shows video frame images (0.87 s, 1.00 s, 1.34 s, and 1.86 s) of dry powder aerodynamic characteristics. The powder flows easily out of the syringe and dissipates quickly in a uniform cloud. Similar behavior in an insufflated rodent model leads to an even distribution.

FIG. 19 shows liver firefly luciferase (FFL) mRNA delivery (0.05 mg/kg dose) of lipids compared to the FDA approved lipid MC3 and RML1, also known as MR-2-93-3.

FIG. 20 shows intramuscular mRNA delivery of amino alcohol headgroup-based lipids compared to MC3.

FIG. 21 shows IN FFL mRNA delivery (0.05 mg/kg dose) compared to control lipids MC-3 and MD-1.

FIGS. 22A-22B show IV FFL mRNA delivery (0.05 mg/kg dose) compared to control lipids SM-102 and MC3. FIG. 22A shows delivery to liver. FIG. 22B shows delivery to spleen.

FIG. 23 shows high-dose IV mRNA delivery (0.8 mg/kg) of RML66 with 20:1 (default) and 15:1 (indicated) ionizable lipid:mRNA weight ratios compared to control lipids Lipid 5 and MC3.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In a formula, the bond [symbol] is a single bond, the dashed line --- is a single bond or absent, and the bond [symbol] or [symbol] is a single or double bond.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₈ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₈ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

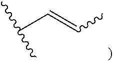

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₈ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C7), cyclooctyl (C₈), cyclooctenyl (C₈), bicyclo[2.2.1]heptanyl (C7), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆-14 aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, −NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂—P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ee}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{f} is, independently, selected from hydrogen, C₁₋₁₀ alkyl, C₁-10 perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NRC^{bb}O₂R^{aa}, —NR^{bb}C(═O)N(R^{bb})₂—NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting ofp-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N—S-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N—S-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl,p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R_{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The disclosure is not intended to be limited in any manner by the above exemplary listing of substituents. Additional terms may be defined in other sections of this disclosure.

### Other Definitions

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N*(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “isotopes” refers to variants of a particular chemical element such that, while all isotopes of a given element share the same number of protons in each atom of the element, those isotopes differ in the number of neutrons.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

As used herein, “lipophilic” refers to the ability of a group to dissolve in fats, oils, lipids, and lipophilic non-polar solvents such as hexane or toluene. In general, a lipophilic group refers to an unsubstituted n-alkyl or unsubstituted n-alkenyl group having 6 to 50 carbon atoms, e.g., 6 to 40, 6 to 30, 6 to 20, 8 to 20, 8 to 19, 8 to 18, 8 to 17, 8 to 16, or 8 to 15 carbon atoms.

The terms “phosphorylethanolamine” and “phosphoethanolamine” are used interchangeably.

The term “sterol” refers to a subgroup of steroids also known as steroid alcohols, i.e., a steroid containing at least one hydroxyl group. Sterols are usually divided into two classes: (1) plant sterols also known as “phytosterols,” and (2) animal sterols also known as “zoosterols.” The term “sterol” includes, but is not limited to, cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers.

As used here, the term “PEG-lipid” refers to a PEGylated lipid.

An “amino acid” refers to natural and unnatural D/L alpha-amino acids, as well as natural and unnatural beta- and gamma-amino acids. A “peptide” refers to two amino acids joined by a peptide bond. A “polypeptide” refers to three or more amino acids joined by peptide bonds. An “amino acid side chain” refers to the group(s) pended to the alpha carbon (if an alpha amino acid), alpha and beta carbon (if a beta amino acid), or the alpha, beta, and gamma carbon (if a gamma amino acid). Exemplary amino acid side chains are depicted herein.

A “protein,” “peptide,” or “polypeptide” comprises a polymer of amino acid residues linked together by peptide bonds. The term, as used herein, refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “apolipoprotein” refers to a protein that binds a lipid (e.g., triacylglycerol or cholesterol) to form a lipoprotein. Apolipoproteins also serve as enzyme cofactors, receptor ligands, and lipid transfer carriers that regulate the metabolism of lipoproteins and their uptake in tissues. Major types of apolipoproteins include integral and non-integral apolipoproteins. Exemplary apolipoproteins include apoA (e.g., apoA-I, apoA-II, apoA-IV, and apoA-V); apoB (e.g., apoB48 and apoB 100); apoC (e.g., apoC-I, apoC-II, apoC-III, and apoC-IV); apoD; apoE; apoH; and apoJ.

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), a provirus, a lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), a polyinosinic acid, a ribozyme, a flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Such promoters include, but are not limited to: the SV40 early promoter region (Bernoist et al., Nature, 290, 304-310, (1981); Yamamoto et al., Cell, 22, 787-797, (1980); Wagner et al., Proc. Natl. Acad. Sci. U.S.A. 78, 1441-1445, (1981); Brinster et al., Nature 296, 39-42, (1982)). Any type of plasmid, cosmid, yeast artificial chromosome or viral vector can be used to prepare the recombinant DNA construct that can be introduced directly into the tissue site. Alternatively, viral vectors can be used which selectively infect the desired tissue, in which case administration may be accomplished by another route (e.g., systemically).

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “recombinant nucleic acid molecule” is a nucleic acid molecule that has undergone a molecular biological manipulation, i.e., non-naturally occurring nucleic acid molecule or genetically engineered nucleic acid molecule. Furthermore, the term “recombinant DNA molecule” refers to a nucleic acid sequence which is not naturally occurring, or can be made by the artificial combination of two otherwise separated segments of nucleic acid sequence, i.e., by ligating together pieces of DNA that are not normally continuous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site specific targets, e.g., promoters, DNA replication sites, regulation sequences, control sequences, open reading frames, or other useful features may be incorporated by design. Examples of recombinant nucleic acid molecule include recombinant vectors, such as cloning or expression vectors which contain DNA sequences encoding Ror family proteins or immunoglobulin proteins which are in a 5′ to 3′ (sense) orientation or in a 3′ to 5′ (antisense) orientation.

The term “pDNA,” “plasmid DNA,” or “plasmid” refers to a small DNA molecule that is physically separate from, and can replicate independently of, chromosomal DNA within a cell. Plasmids can be found in all three major domains: Archaea, Bacteria, and Eukarya. In nature, plasmids carry genes that may benefit survival of the subject (e.g., antibiotic resistance) and can frequently be transmitted from one bacterium to another (even of another species) via horizontal gene transfer. Artificial plasmids are widely used as vectors in molecular cloning, serving to drive the replication of recombinant DNA sequences within host subjects. Plasmid sizes may vary from 1 to over 1,000 kbp. Plasmids are considered replicons, capable of replicating autonomously within a suitable host.

“RNA transcript” refers to the product resulting from RNA polymerase-catalyzed transcription of a DNA sequence. When the RNA transcript is a complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “gene silencing” refers to an epigenetic process of gene regulation where a gene is “switched off” by a mechanism other than genetic modification. That is, a gene which would be expressed (i.e., “turned on”) under normal circumstances is switched off by machinery in the cell. Gene silencing occurs when RNA is unable to make a protein during translation. Genes are regulated at either the transcriptional or post-transcriptional level. Transcriptional gene silencing is the result of histone modifications, creating an environment of heterochromatin around a gene that makes it inaccessible to transcriptional machinery (e.g., RNA polymerase and transcription factors). Post-transcriptional gene silencing is the result of mRNA of a particular gene being destroyed or blocked. The destruction of the mRNA prevents translation and thus the formation of a gene product (e.g., a protein). A common mechanism of post-transcriptional gene silencing is RNAi.

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles (e.g., lipid nanoparticles), macroscopic particles, pellets, crystals, aggregates, composites, pulverized, milled or otherwise disrupted matrices, and cross-linked protein or polysaccharide particles, each of which have an average characteristic dimension of about less than about 1 mm and at least 1 nm, where the characteristic dimension, or “critical dimension,” of the particle is the smallest cross-sectional dimension of the particle. A particle may be composed of a single substance or multiple substances. In certain embodiments, the particle is not a viral particle. In other embodiments, the particle is not a liposome. In certain embodiments, the particle is not a micelle. In certain embodiments, the particle is substantially solid throughout. In certain embodiments, the particle is a nanoparticle. In certain embodiments, the particle is a microparticle.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound or agent described herein refers to an amount sufficient to elicit the desired biological response. An effective amount of a compound or agent described herein may vary depending on such factors as the desired biological endpoint, severity of side effects, disease, or disorder, the identity, pharmacokinetics, and pharmacodynamics of the particular compound or agent, the condition being treated, the mode, route, and desired or required frequency of administration, the species, age and health or general condition of the subject. In certain embodiments, an effective amount is a therapeutically effective amount. In certain embodiments, an effective amount is a prophylactically effective amount. In certain embodiments, an effective amount is the amount of a compound or agent described herein in a single dose. In certain embodiments, an effective amount is the combined amounts of a compound or agent described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound or agent described herein is an amount sufficient to provide a therapeutic benefit in the treatment of a condition or to delay or minimize one or more symptoms associated with the condition. A therapeutically effective amount of a compound or agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for delivering an agent to a subject or a cell. In certain embodiments, a therapeutically effective amount is an amount sufficient for delivering a polynucleotide to a subject or a cell. In certain embodiments, a therapeutically effective amount is an amount sufficient for delivering mRNA to a subject or a cell. In certain embodiments, a therapeutically effective amount is an amount sufficient for treating a disease, disorder, or condition. In certain embodiments, a therapeutically effective amount is an amount sufficient for delivering an agent to a subject or a cell and treating a disease, disorder, or condition. In certain embodiments, a therapeutically effective amount is an amount sufficient for delivering a polynucleotide to a subject or a cell and treating a disease, disorder, or condition. In certain embodiments, a therapeutically effective amount is an amount sufficient for delivering mRNA to a subject or a cell and treating a disease, disorder, or condition.

A “prophylactically effective amount” of a compound or agent described herein is an amount sufficient to prevent a condition, or one or more symptoms associated with the condition or prevent its recurrence. A prophylactically effective amount of a compound or agent means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for delivering an agent to a subject or a cell. In certain embodiments, a prophylactically effective amount is an amount sufficient for delivering a polynucleotide to a subject or a cell. In certain embodiments, a prophylactically effective amount is an amount sufficient for delivering mRNA to a subject or a cell. In certain embodiments, a prophylactically effective amount is an amount sufficient for preventing a disease, disorder, or condition. In certain embodiments, a prophylactically effective amount is an amount sufficient for delivering an agent to a subject or a cell and preventing a disease, disorder, or condition. In certain embodiments, a prophylactically effective amount is an amount sufficient for delivering a polynucleotide to a subject or a cell and preventing a disease, disorder, or condition. In certain embodiments, a prophylactically effective amount is an amount sufficient for delivering mRNA to a subject or a cell and preventing a disease, disorder, or condition.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “neuronal disease” refers to diseases of the central and/or peripheral nervous system associated with neuronal degeneration or damage. Specific examples of neuronal diseases are characterized by neuronal necrosis or loss, for example, associated with Alzheimer's disease, Parkinson's disease, Huntington's chorea, seizures, ALS, peripheral neuropathy, and central, peripheral or motor neurons.

The term “infectious disease” refers to a disease caused by pathogenic microorganisms residing and proliferating in the tissue, body fluid, or surface of a host (e.g., a human, animal, or plant). Infectious diseases can be divided into different types according to the route of infection and contagiousness. Infectious diseases include, but are not limited to diseases arising from viral infection, fungal infection, bacterial infection, protozoan infection, or parasitic infection.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

The term “lung disease” or “pulmonary disease” refers to a disease of the lung. Examples of lung diseases include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

A “painful condition” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g., nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy; neuralgia (e.g., post-herpetic neuralgia, trigeminal neuralgia); pain resulting from physical trauma (e.g., amputation; surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression; nerve crush, nerve stretch or incomplete nerve transsection; mononeuropathy or polyneuropathy. Neuropathic pain can also result from a disorder such as dorsal root ganglion compression; inflammation of the spinal cord; contusion, tumor or hemisection of the spinal cord; tumors of the brainstem, thalamus or cortex; or trauma to the brainstem, thalamus or cortex.

The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia). [00449] In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulus of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “musculoskeletal disease” or “MSD” refers to an injury and/or pain in a subject's joints, ligaments, muscles, nerves, tendons, and structures that support limbs, neck, and back. In certain embodiments, an MSD is a degenerative disease. In certain embodiments, an MSD includes an inflammatory condition. Body parts of a subject that may be associated with MSDs include upper and lower back, neck, shoulders, and extremities (arms, legs, feet, and hands). In certain embodiments, an MSD is a bone disease, such as achondroplasia, acromegaly, bone callus, bone demineralization, bone fracture, bone marrow disease, bone marrow neoplasm, dyskeratosis congenita, leukemia (e.g., hairy cell leukemia, lymphocytic leukemia, myeloid leukemia, Philadelphia chromosome-positive leukemia, plasma cell leukemia, stem cell leukemia), systemic mastocytosis, myelodysplastic syndromes, paroxysmal nocturnal hemoglobinuria, myeloid sarcoma, myeloproliferative disorders, multiple myeloma, polycythemia vera, pearson marrow-pancreas syndrome, bone neoplasm, bone marrow neoplasm, Ewing sarcoma, osteochondroma, osteoclastoma, osteosarcoma, brachydactyly, Camurati-Engelmann syndrome, Craniosynostosis, Crouzon craniofacial dysostosis, dwarfism, achondroplasia, bloom syndrome, Cockayne syndrome, Ellis-van Creveld syndrome, Seckel syndrome, spondyloepiphyseal dysplasia, spondyloepiphyseal dysplasia congenita, Werner syndrome, hyperostosis, osteophyte, Klippel-Trenaunay-Weber syndrome, Marfan syndrome, McCune-Albright syndrome, osteitis, osteoarthritis, osteochondritis, osteochondrodysplasia, Kashin-Beck disease, Leri-Weill dyschondrosteosis, osteochondrosis, osteodystrophy, osteogenesis imperfecta, osteolysis, Gorham-Stout syndrome, osteomalacia, osteomyelitis, osteonecrosis, osteopenia, osteopetrosis, osteoporosis, osteosclerosis, otospondylomegaepiphyseal dysplasia, pachydermoperiostosis, Paget disease of bone, Polydactyly, Meckel syndrome, rickets, Rothmund-Thomson syndrome, Sotos syndrome, spondyloepiphyseal dysplasia, spondyloepiphyseal dysplasia congenita, syndactyly, Apert syndrome, syndactyly type II, or Werner syndrome. In certain embodiments, an MSD is a cartilage disease, such as cartilage neoplasm, osteochondritis, osteochondrodysplasia, Kashin-Beck disease, or Leri-Weill dyschondrosteosis. In certain embodiments, an MSD is hernia, such as intervertebral disk hernia. In certain embodiments, an MSD is a joint disease, such as arthralgia, arthritis (e.g., gout (e.g., Kelley-Seegmiller syndrome, Lesch-Nyhan syndrome), Lyme disease, osteoarthritis, psoriatic arthritis, reactive arthritis, rheumatic fever, rheumatoid arthritis, Felty syndrome, synovitis, Blau syndrome, nail-patella syndrome, spondyloarthropathy, reactive arthritis, Stickler syndrome, synovial membrane disease, synovitis, or Blau syndrome. In certain embodiments, an MSD is Langer-Giedion syndrome. In certain embodiments, an MSD is a muscle disease, such as Barth syndrome, mitochondrial encephalomyopathy, MELAS syndrome, MERRF syndrome, MNGIE syndrome, mitochondrial myopathy, Kearns-Sayre syndrome, myalgia, fibromyalgia, polymyalgia rheumatica, myoma, myositis, dermatomyositis, neuromuscular disease, Kearns-Sayre syndrome, muscular dystrophy, myasthenia, congenital myasthenic syndrome, Lambert-Eaton myasthenic syndrome, myasthenia gravis, myotonia, myotonia congenita, spinal muscular atrophy, tetany, ophthalmoplegia, or rhabdomyolysis. In certain embodiments, an MSD is Proteus syndrome. In certain embodiments, an MSD is a rheumatic diseases, such as arthritis (e.g., gout (e.g., Kelley-Seegmiller syndrome, Lesch-Nyhan lyme disease)), osteoarthritis, psoriatic arthritis, reactive arthritis, rheumatic fever, rheumatoid arthritis, Felty syndrome, synovitis, Blau syndrome, gout (e.g., Kelley-Seegmiller syndrome, Lesch-Nyhan syndrome), polymyalgia rheumatica, rheumatic fever, rheumatic heart disease, or Sjogren syndrome. In certain embodiments, an MSD is Schwartz-Jampel syndrome. In certain embodiments, an MSD is a skeleton disease, such as Leri-Weill dyschondrosteosis, skeleton malformations, Melnick-Needles syndrome, pachydermoperiostosis, Rieger syndrome, spinal column disease, intervertebral disk hernia, scoliosis, spina bifida, spondylitis, ankylosing spondylitis, spondyloarthropathy, reactive arthritis, spondyloepiphyseal dysplasia, spondyloepiphyseal dysplasia congenita, or spondylosis.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity.

In certain embodiments, the metabolic disorder is a wasting condition. A “wasting condition” includes but is not limited to, anorexia and cachexias of various natures (e.g., weight loss associated with cancer, weight loss associated with other general medical conditions, weight loss associated with failure to thrive, and the like). In certain embodiments, the metabolic disorder is an obesity-related condition or a complication thereof. An “obesity-related condition” includes, but is not limited to, obesity, undesired weight gain (e.g., from medication-induced weight gain, from cessation of smoking) and an over-eating disorder (e.g., binge eating, bulimia, compulsive eating, or a lack of appetite control each of which can optionally lead to undesired weight gain or obesity). “Obesity” and “obese” refers to class I obesity, class II obesity, class III obesity and pre-obesity (e.g., being “over-weight”) as defined by the World Health Organization.

Reduction of storage fat is expected to provide various primary and/or secondary benefits in a subject (e.g., in a subject diagnosed with a complication associated with obesity) such as, for example, an increased insulin responsiveness (e.g., in a subject diagnosed with Type II diabetes mellitus); a reduction in elevated blood pressure; a reduction in elevated cholesterol levels; and/or a reduction (or a reduced risk or progression) of ischemic heart disease, arterial vascular disease, angina, myocardial infarction, stroke, migraines, congestive heart failure, deep vein thrombosis, pulmonary embolism, gall stones, gastroesophagael reflux disease, obstructive sleep apnea, obesity hypoventilation syndrome, asthma, gout, poor mobility, back pain, erectile dysfunction, urinary incontinence, liver injury (e.g., fatty liver disease, liver cirrhosis, alcoholic cirrhosis, endotoxin mediated liver injury) or chronic renal failure.

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease).

The term “allergic disease” refers to a hypersensitivity disorder of the immune system, such as, but not limited to, allergic rhinitis, allergic conjunctivitis, allergic bronchial asthma, atopic eczema, anaphylaxis, insect bite, drug allergies, food allergies, allergic eye disease or multiple allergies (such as asthma, eczema and allergic rhinitis together). In some embodiments, the allergic disease is seasonal. In some embodiments, the allergic disease is perennial.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In certain embodiments, the inflammatory disorder and/or the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are compounds (e.g., compounds of Formulae (I) and (II)), and pharmaceutically acceptable salts, solvates, tautomers, stereoisomers, and isotopically labeled derivatives thereof, and compositions and kits thereof. The compounds provided herein can form particles and may therefore be used to deliver agents (e.g., a polynucleotide) to a subject, target tissue, or cell. Also provided herein are methods of delivery and methods of treating a disease, disorder, or condition, comprising administering to the subject a composition comprising a compound provided herein (e.g., a compound of Formulae (I) or (II)), or a pharmaceutically acceptable salt, solvate, tautomer, stereoisomer, or isotopically labeled derivative thereof. Methods of synthesis of a compound provided herein (e.g., a compound of Formulae (I) or (II)), or a pharmaceutically acceptable salt, solvate, tautomer, stereoisomer, or isotopically labeled derivative thereof, are also provided.

### Compounds

Provided herein are compounds of Formula (I):

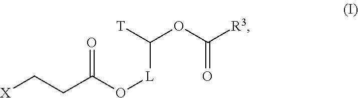

or a pharmaceutically acceptable salt, solvate, tautomer, stereoisomer, or isotopically labeled derivative thereof, wherein:
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl;
T is optionally substituted C₁-C₂₀ alkyl, optionally substituted C₂-C₂₀ alkenyl, or optionally substituted C₂-C₂₀ alkynyl;
L is optionally substituted C₁-C₂₀ alkylene, optionally substituted C₂-C₂₀ alkenylene, or optionally substituted C₂-C₂₀ alkynylene; and
R³ is optionally substituted aliphatic or optionally substituted heteroaliphatic.

As defined herein, X is —OR¹, —SR¹, or —NR¹R². In certain embodiments, X is —OR¹, or —SR¹. In some embodiments, X is —SR¹ or —NR¹R². In certain embodiments, X is —OR¹ or —NR¹R². In some embodiments, X is —OR¹. In certain embodiments, X is —SR¹. In some embodiments, X is —NR¹R².

As defined herein, T is optionally substituted C₁-C₂₀ alkyl, optionally substituted C₂-C₂₀ alkenyl, or optionally substituted C₂-C₂₀ alkynyl. In some embodiments, T is unsubstituted C₁-C₂₀ alkyl, unsubstituted C₂-C₂₀ alkenyl, or unsubstituted C₂-C₂₀ alkynyl. In some embodiments, T is unsubstituted C₁-C₂₀ alkyl or unsubstituted C₂-C₂₀ alkenyl. In some embodiments, T is unsubstituted C₁-C₂₀ alkyl. In some embodiments, T is unsubstituted C₁-C₁₀ alkyl. In some embodiments, T is unsubstituted C₅-C₁₀ alkyl. In some embodiments, T is unsubstituted C₁-C₂₀ alkenyl. In some embodiments, T is unsubstituted C₁-C₁₀ alkenyl. In some embodiments, T is unsubstituted C₅-C₁₀ alkenyl. In some embodiments, T is unsubstituted C₁-C₂₀ alkenyl with one double bond. In some embodiments, T is unsubstituted C₁-C₁₀ alkenyl with one double bond. In some embodiments, T is unsubstituted C₅-C₁₀ alkenyl with one double bond.

In some embodiments, T is selected from:

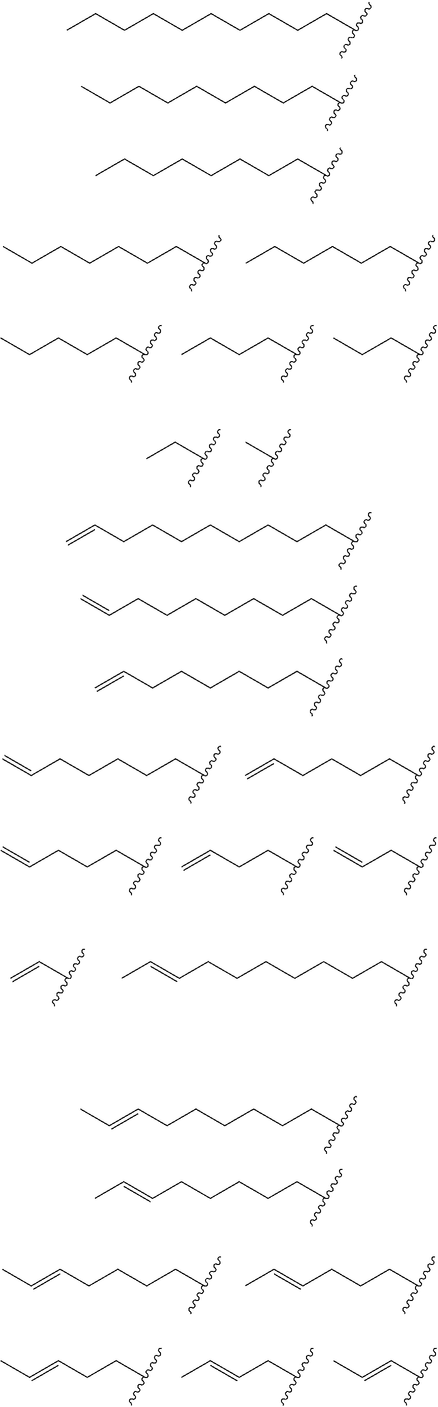

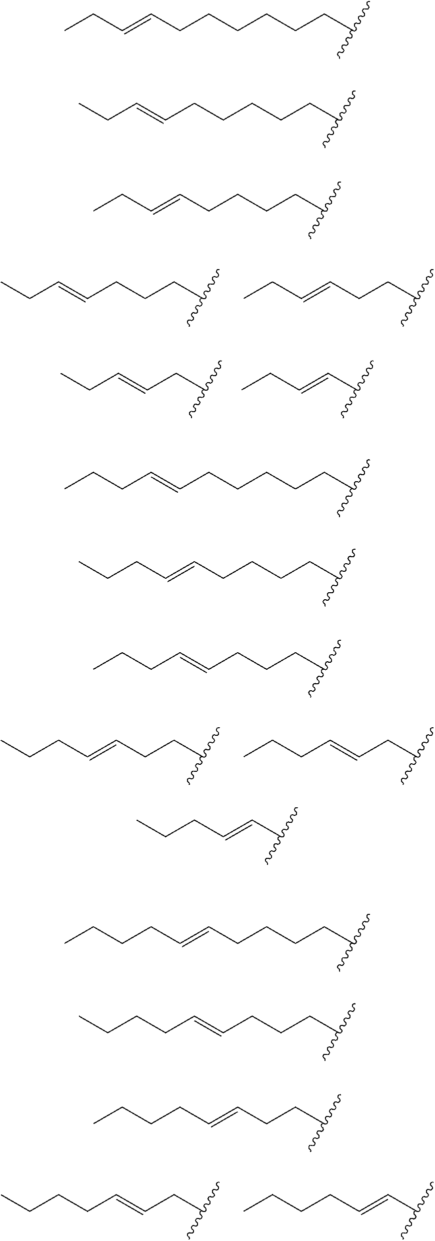

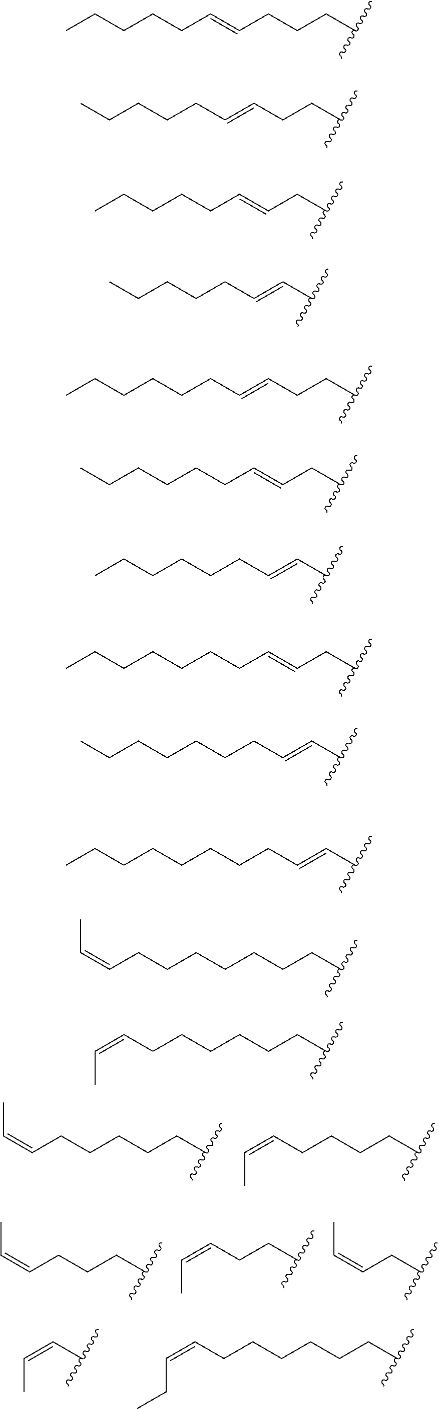

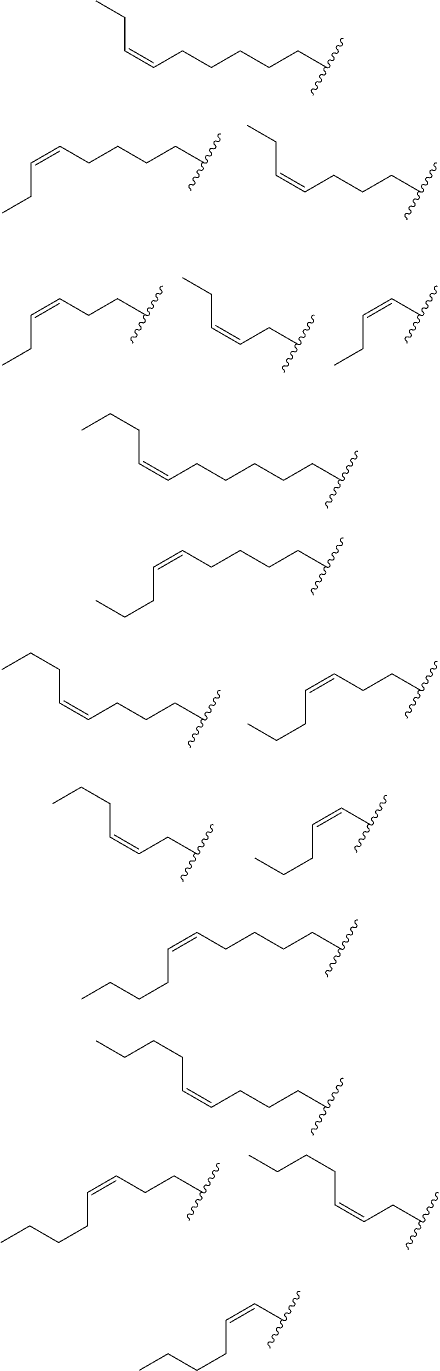

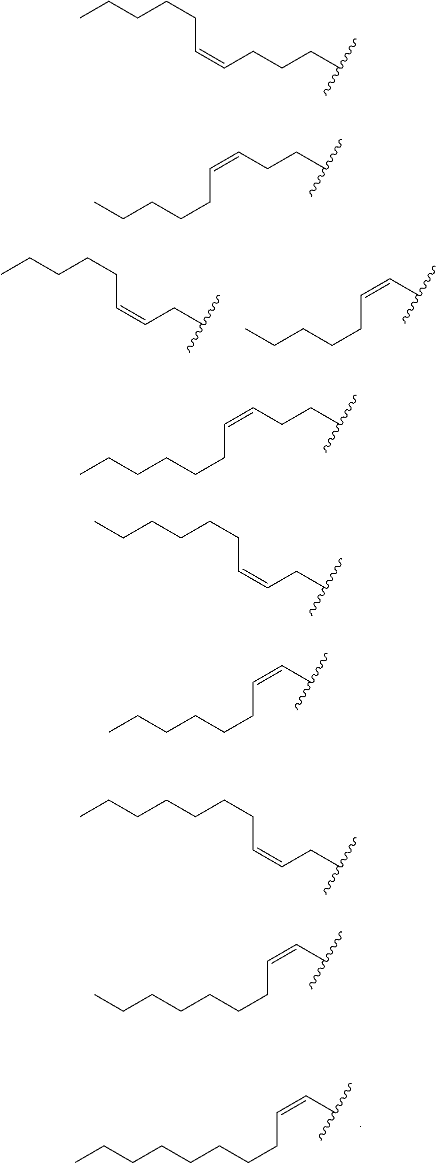

In some embodiments, T is selected from

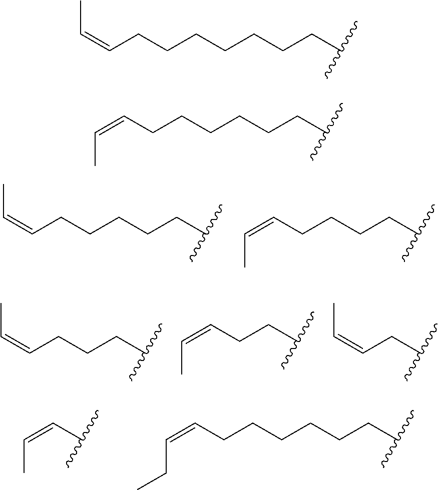

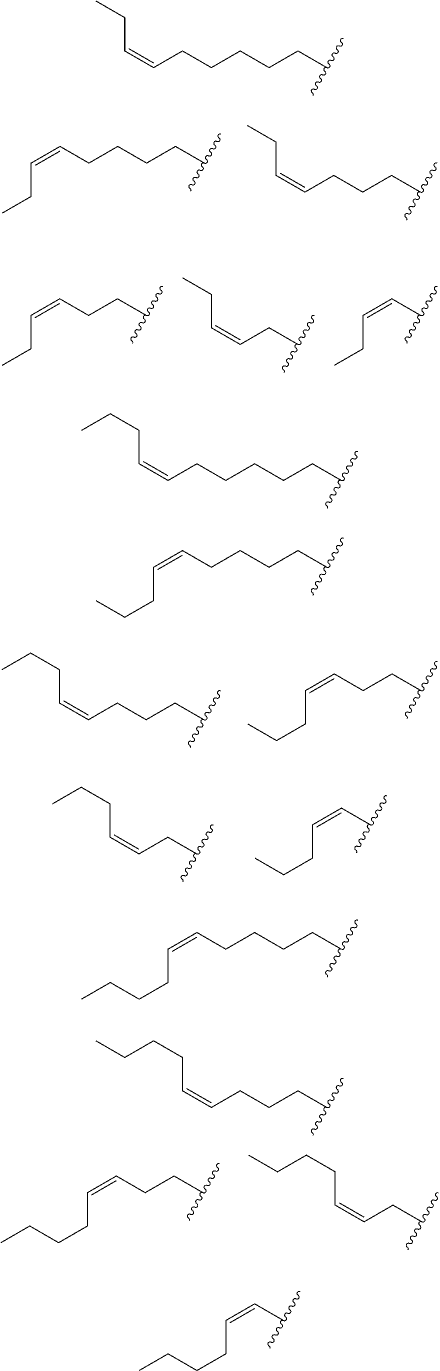

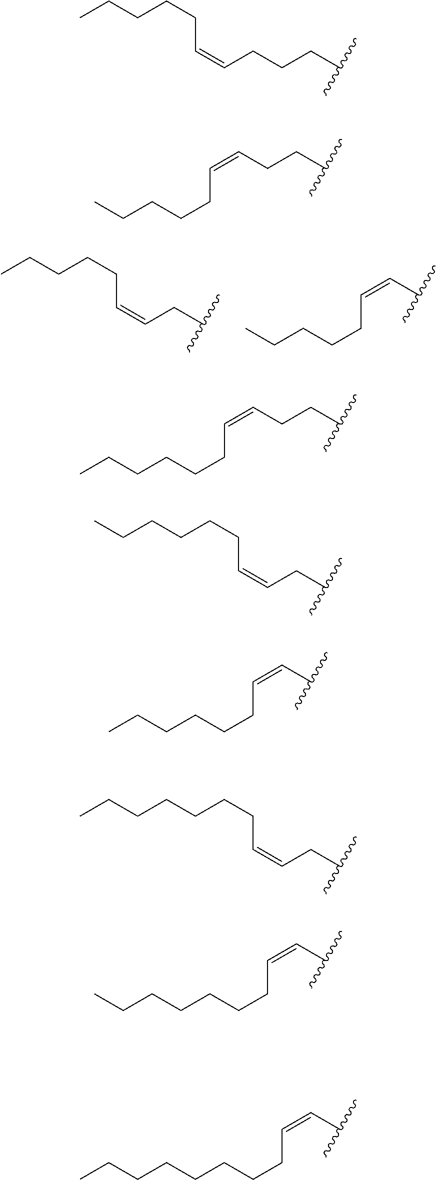

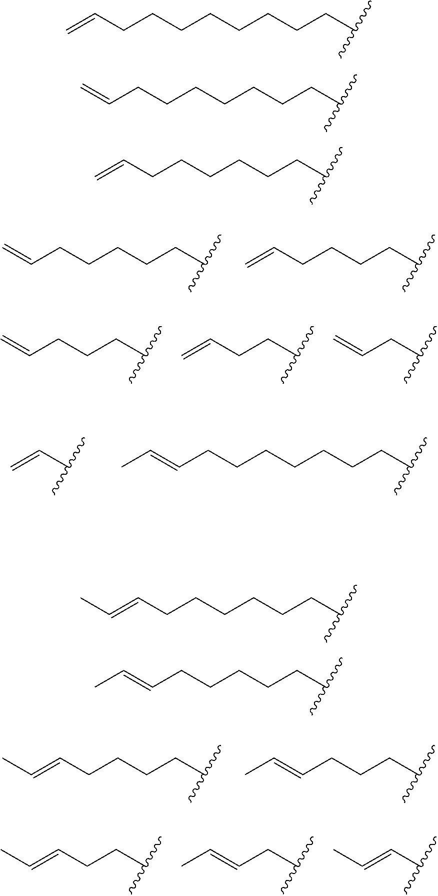

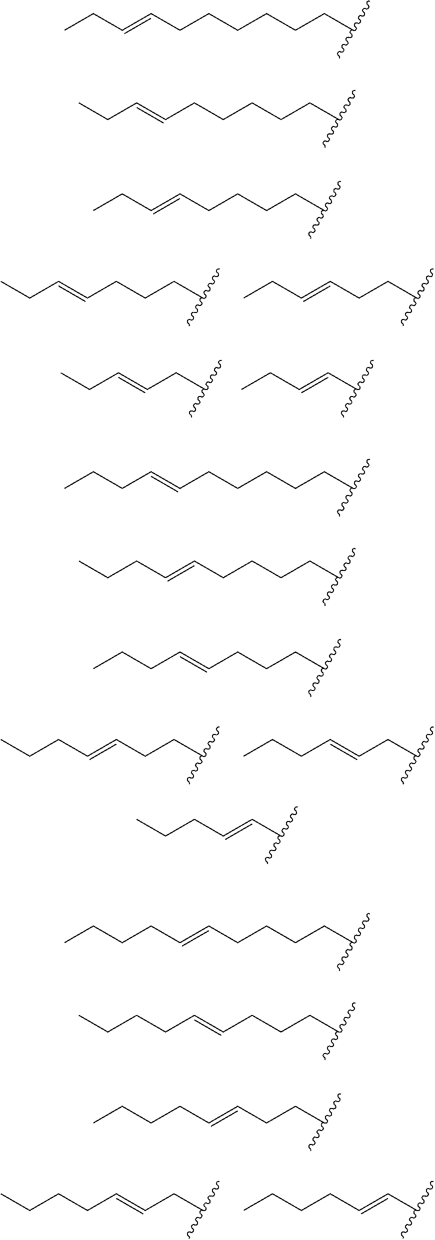

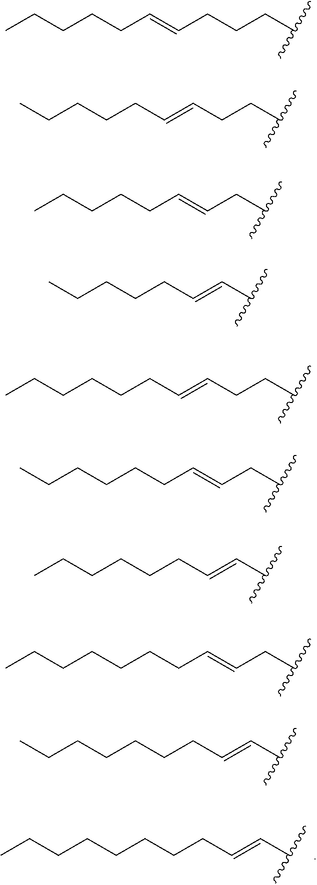

In some embodiments, T is selected from

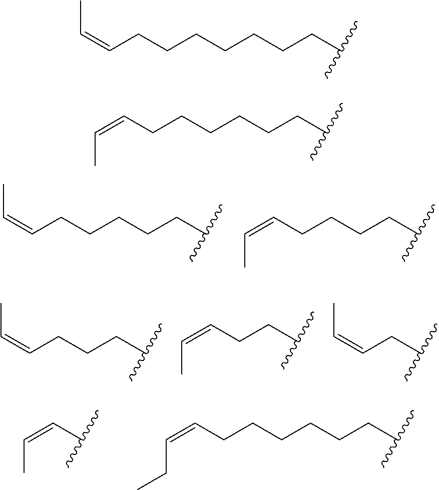

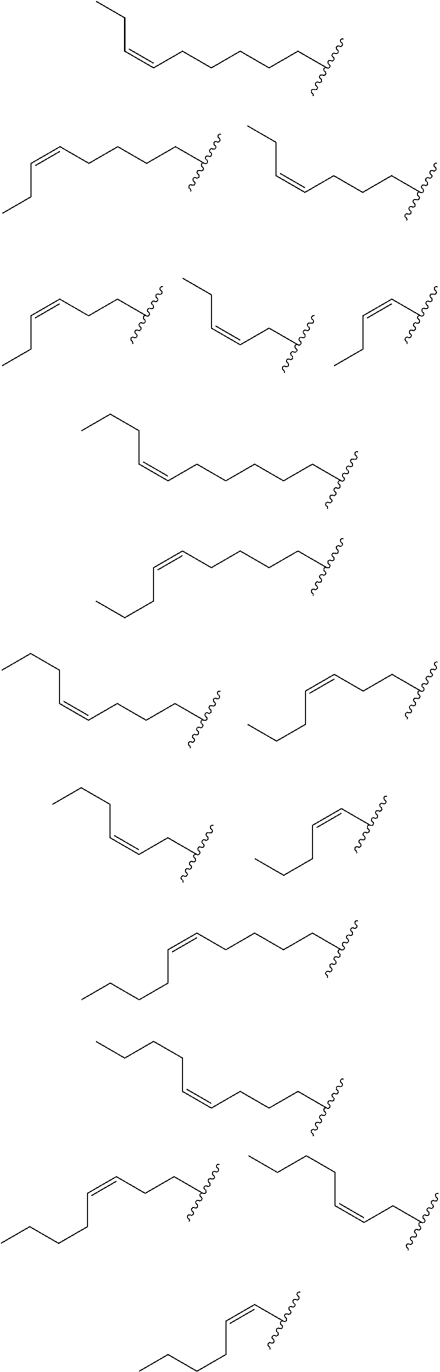

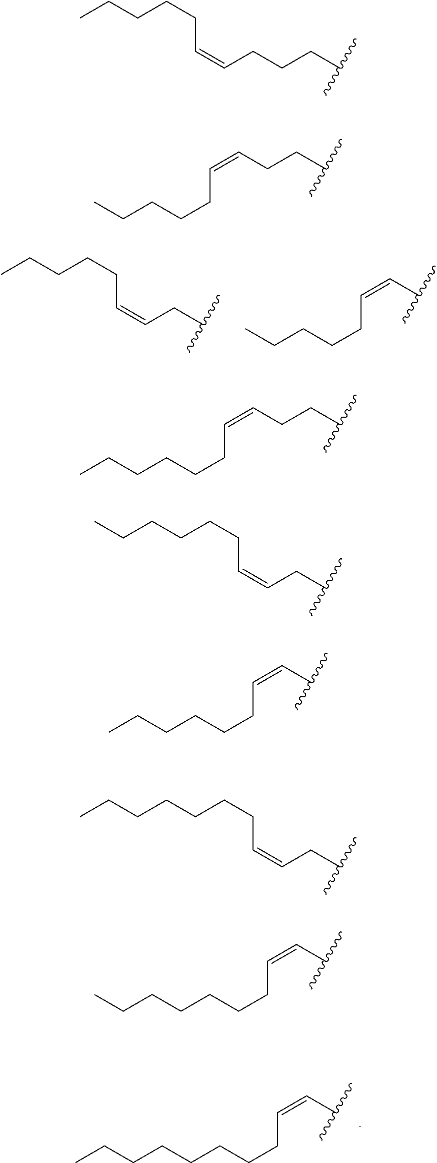

In some embodiments, T is selected from

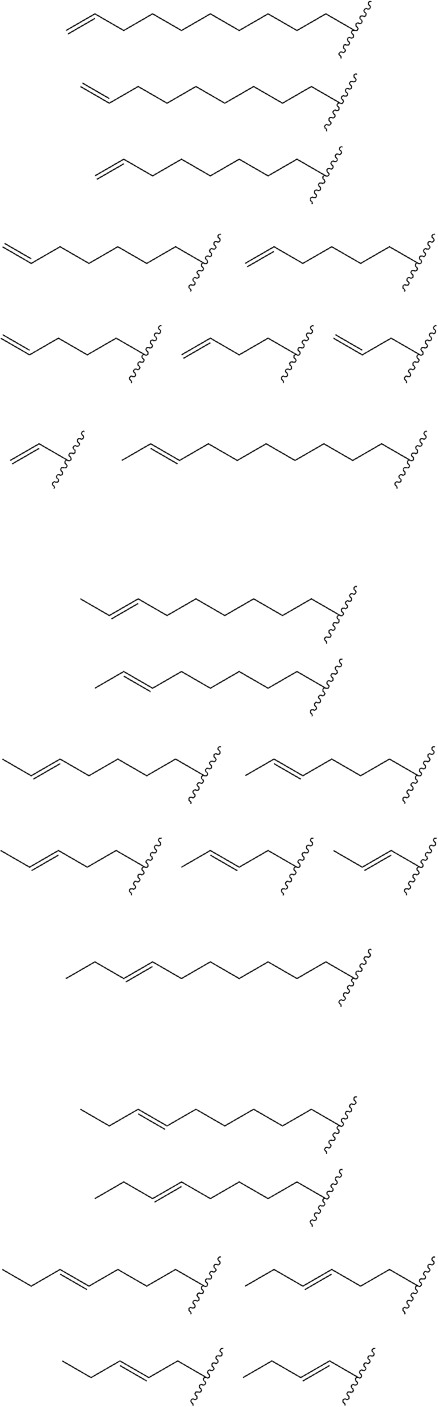

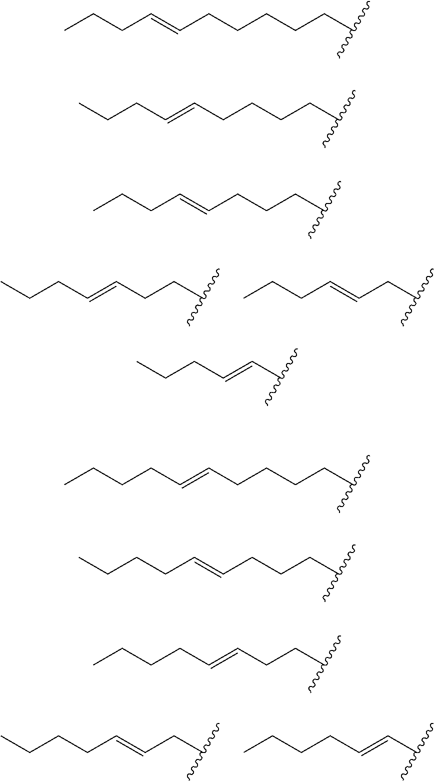

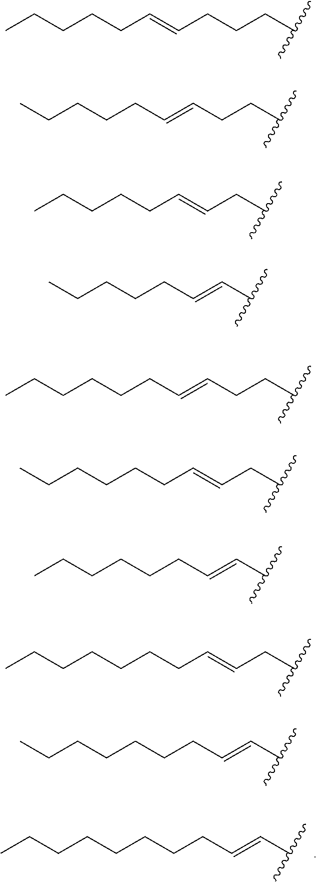

In some embodiments T is selected from

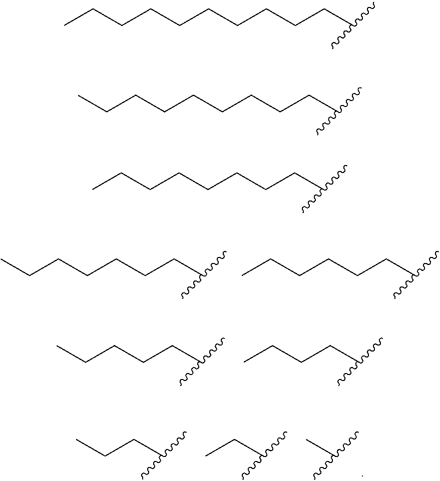

In some embodiments, T is

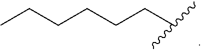 [SMILES: *CCCCCC]

As defined herein, L is optionally substituted C₁-C₂₀ alkylene, optionally substituted C₂-C₂₀ alkenylene, or optionally substituted C₂-C₂₀ alkynylene. In some embodiments, L is unsubstituted C₁-C₂₀ alkylene, unsubstituted C₂-C₂₀ alkenylene, or unsubstituted C₂-C₂₀ alkynylene. In some embodiments, L is unsubstituted C₁-C₂₀ alkylene or unsubstituted C₂-C₂₀ alkenylene. In some embodiments, L is unsubstituted C₅-C₁₅ alkylene or unsubstituted C₅-C₁₅ alkenylene. In some embodiments, L is unsubstituted C₁-C₂₀ alkylene. In some embodiments, L is unsubstituted C₅-C₁₅ alkylene. In some embodiments, L is unsubstituted C₉-C₁₃ alkylene. In some embodiments, L is unsubstituted C₂-C₂₀ alkenylene. In some embodiments, L is unsubstituted C₅-C₁₅ alkenylene. In some embodiments, L is unsubstituted C₉-C₁₃ alkenylene. In some embodiments, L is unsubstituted C₂-C₂₀ alkenylene with one double bond. In some embodiments, L is unsubstituted C₅-C₁₅ alkenylene with one double bond. In some embodiments, L is unsubstituted C₉-C₁₃ alkenylene with one double bond.

In some embodiments, L is selected from:

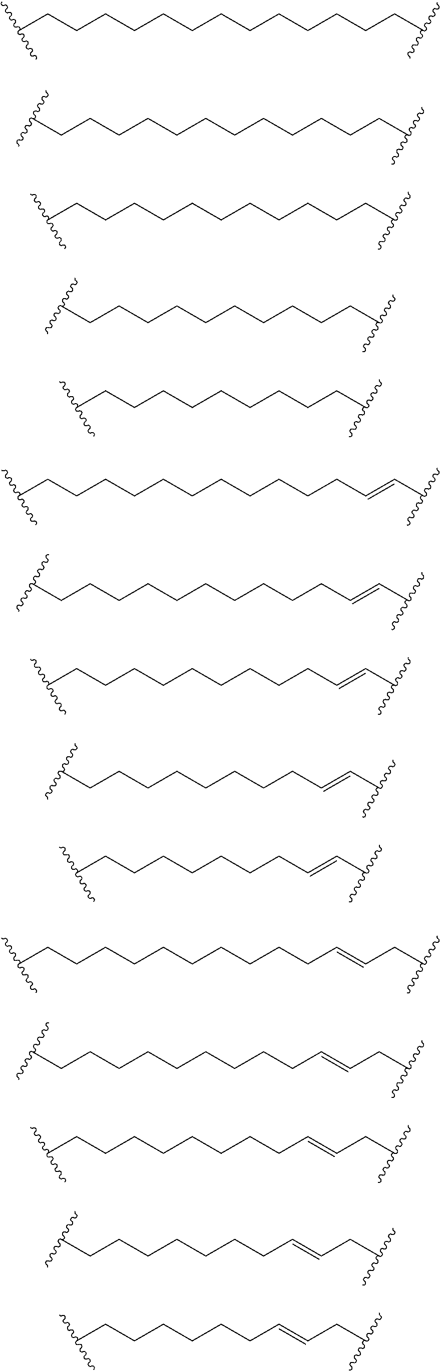

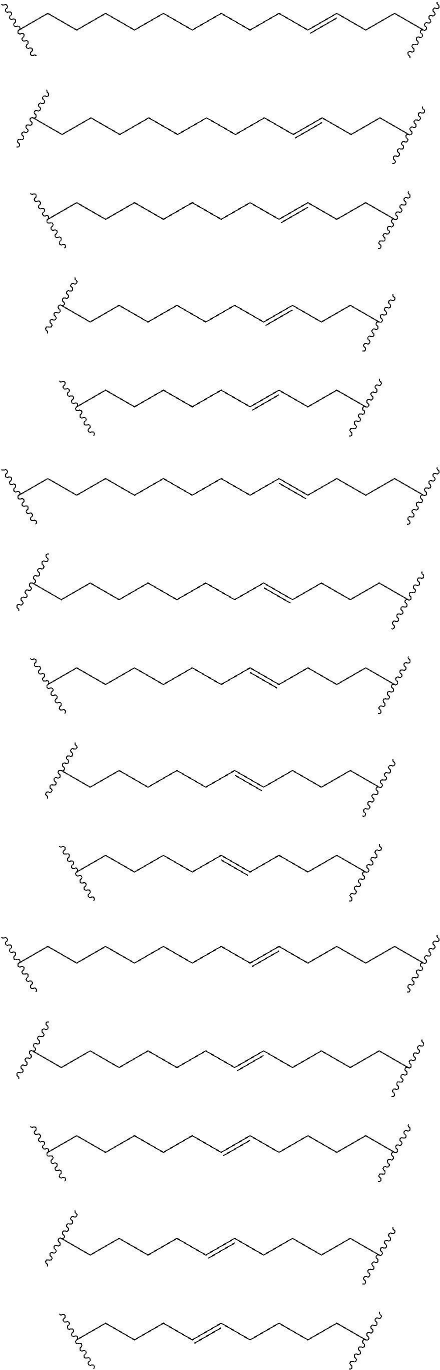

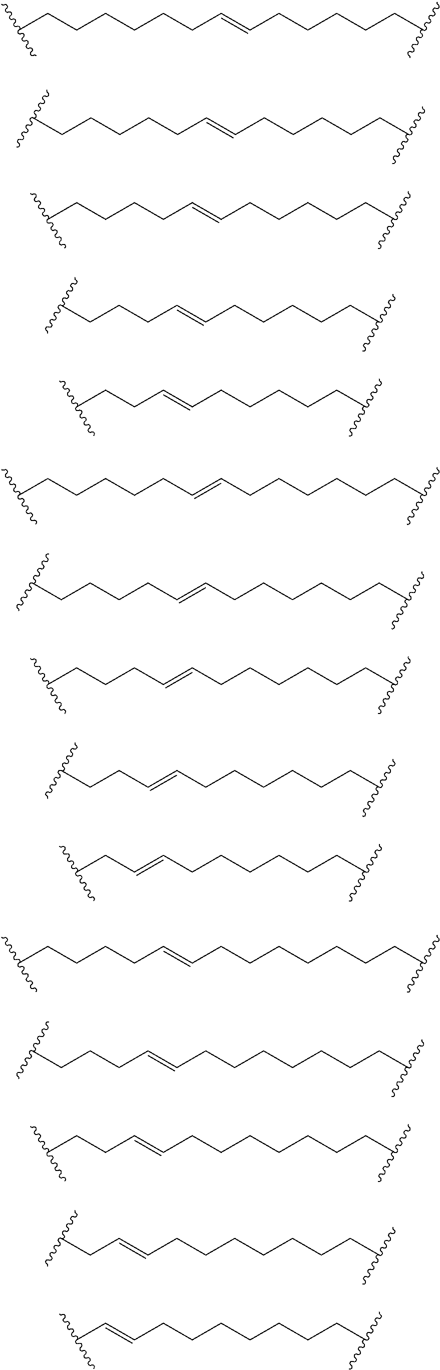

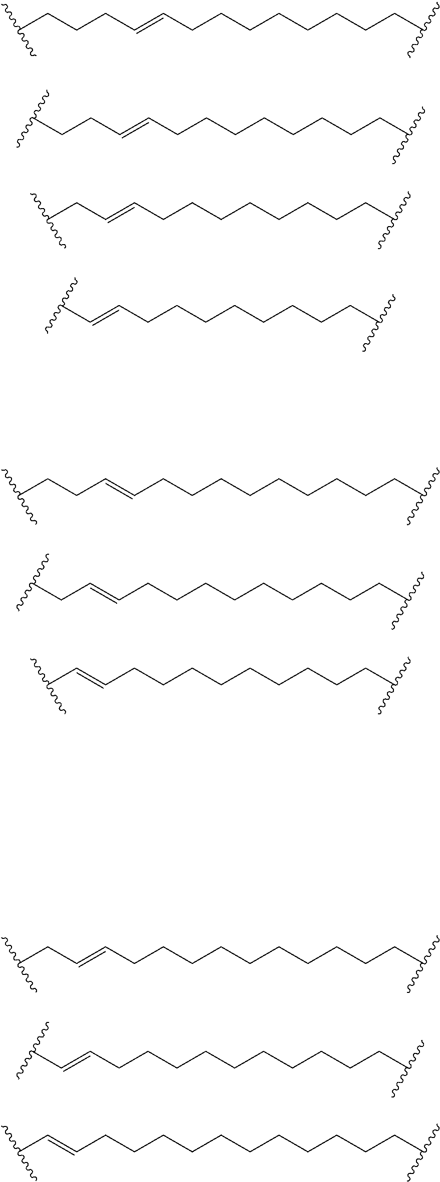

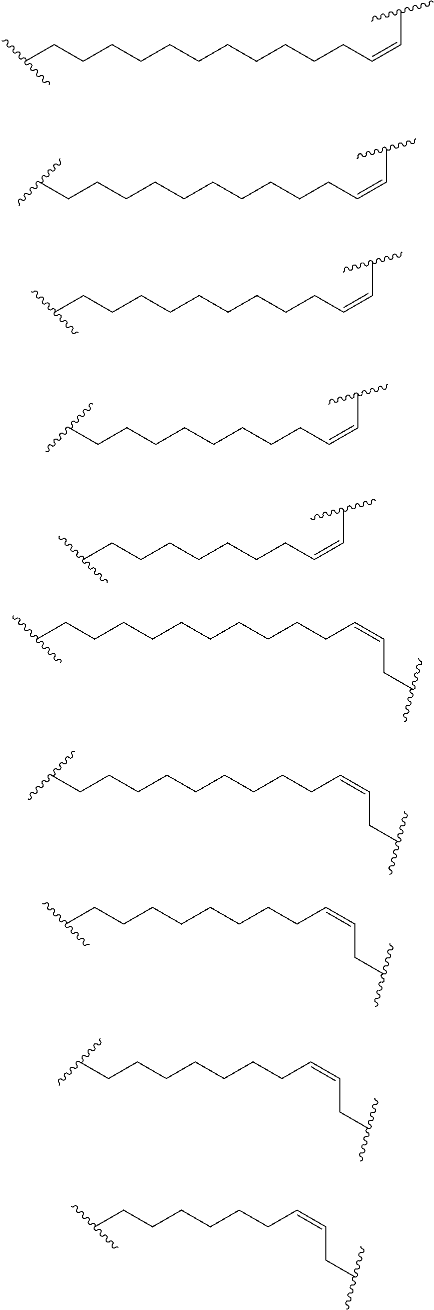

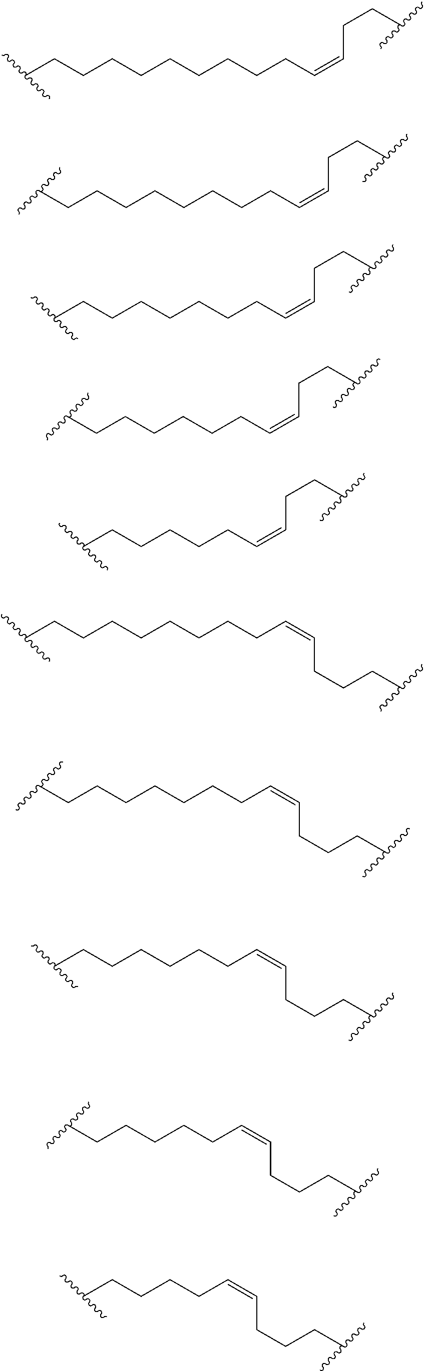

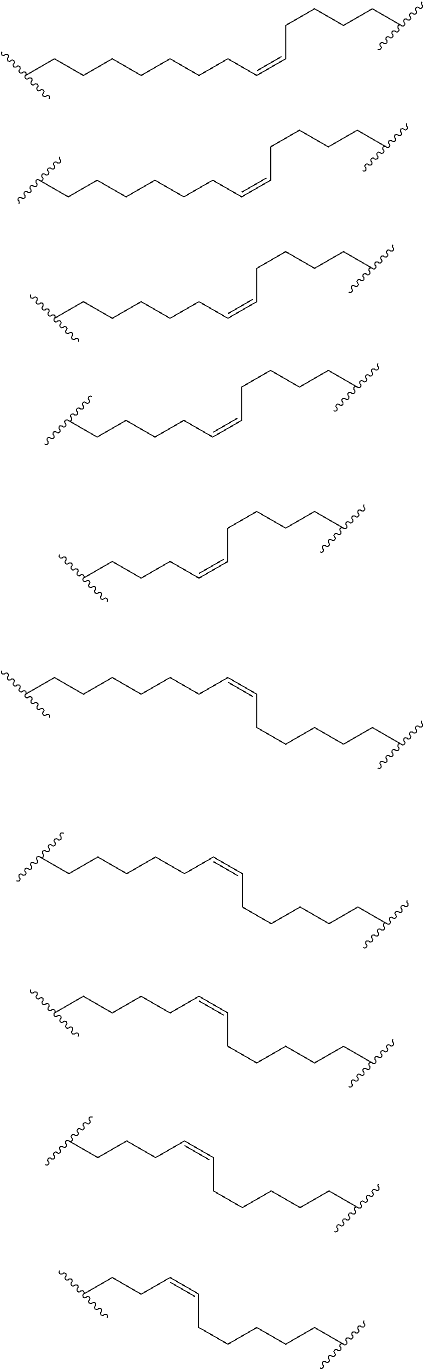

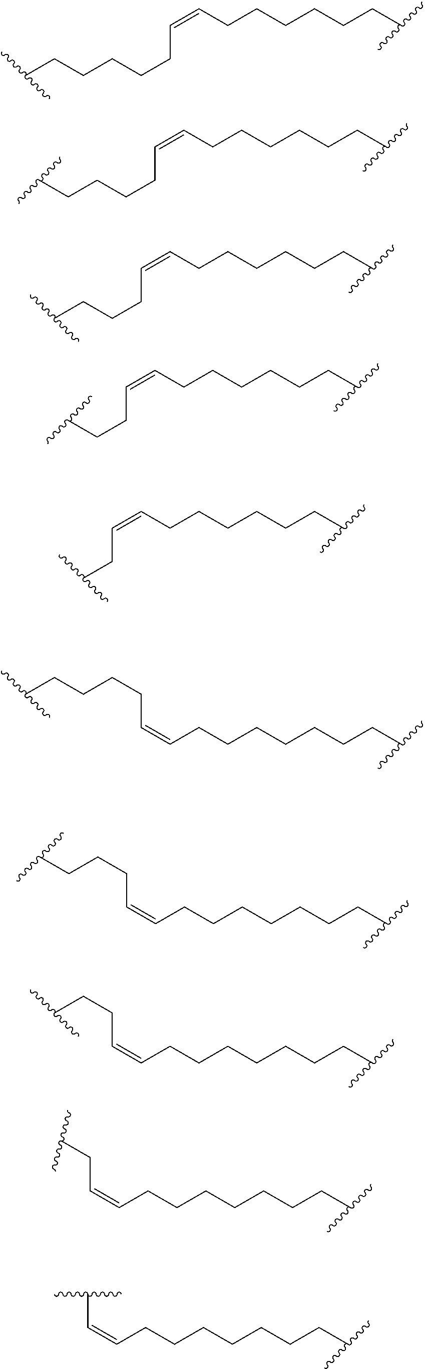

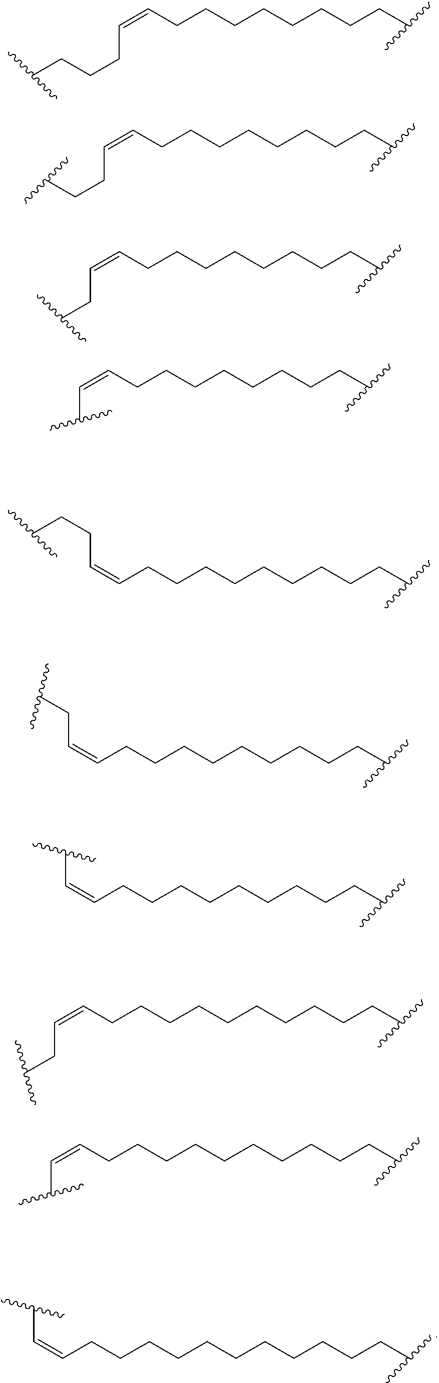

In some embodiments, L is selected from:

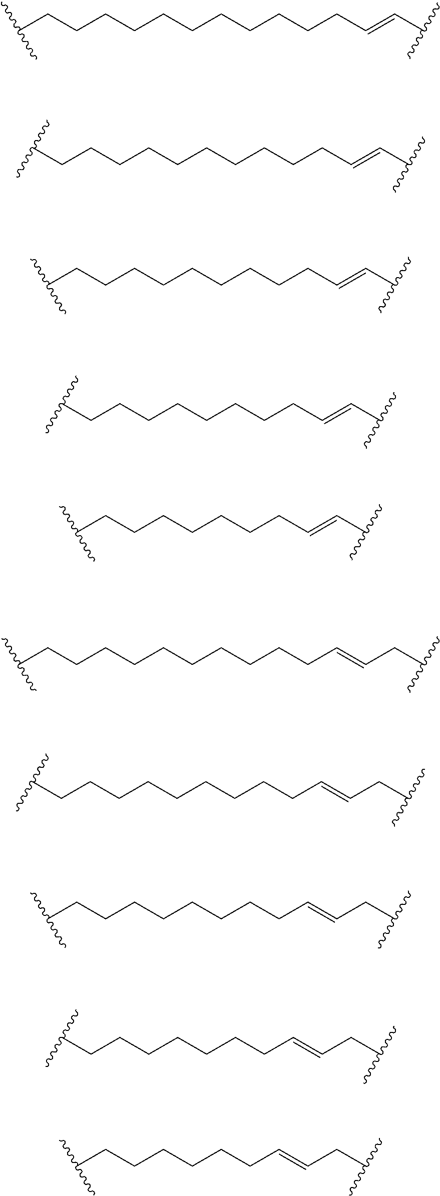

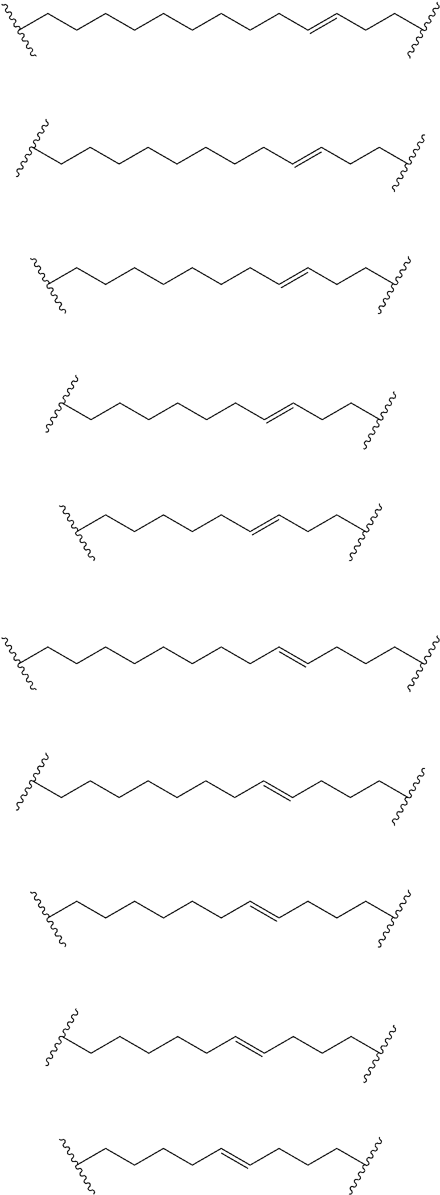

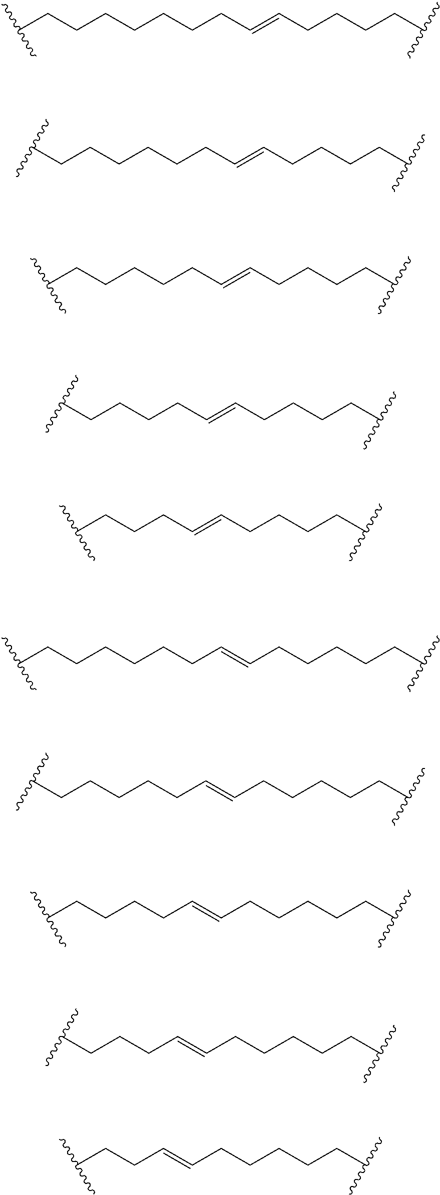

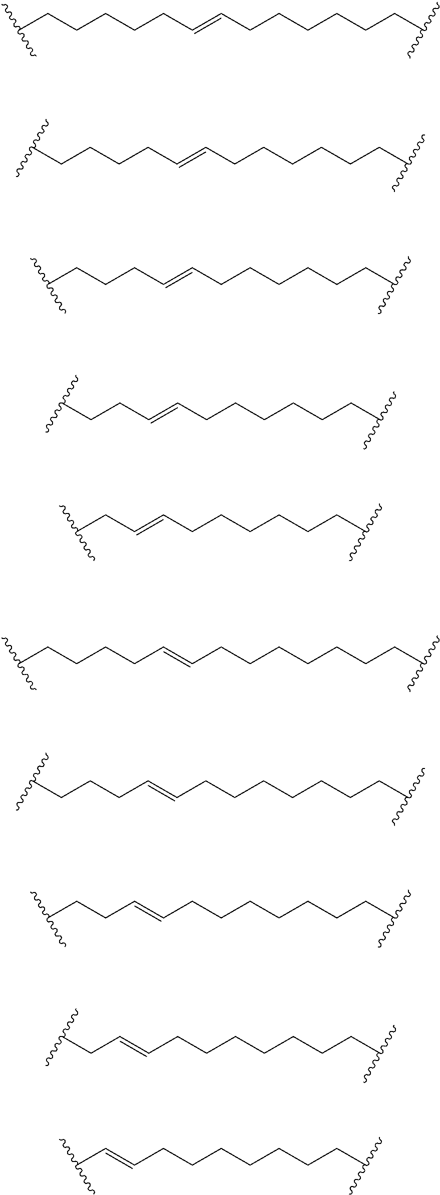

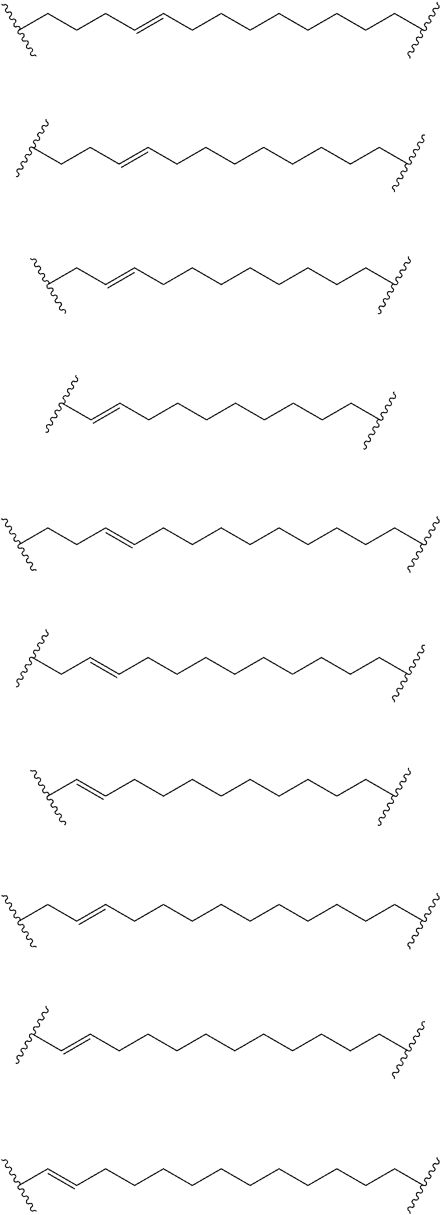

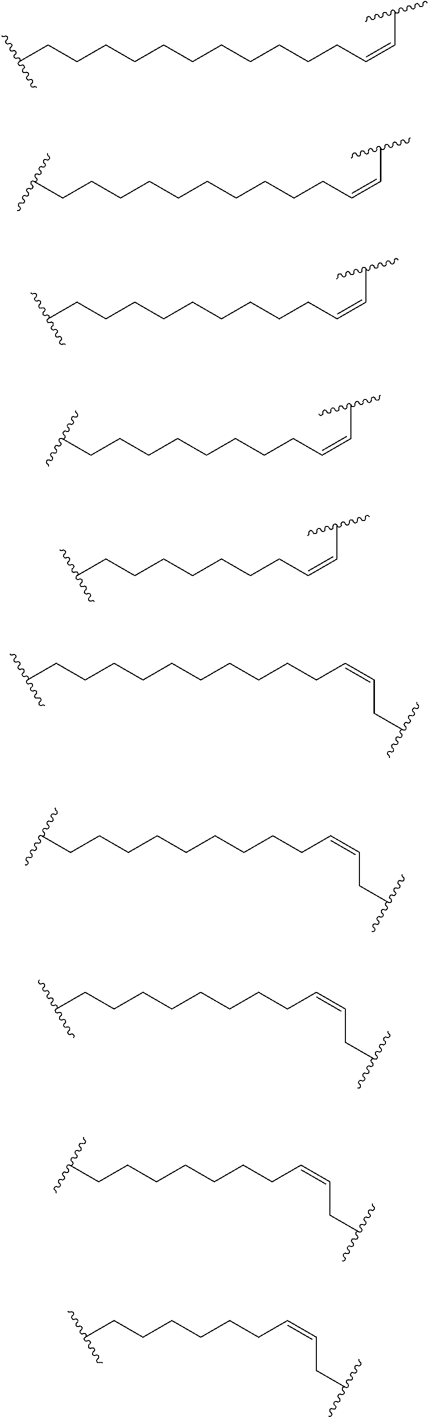

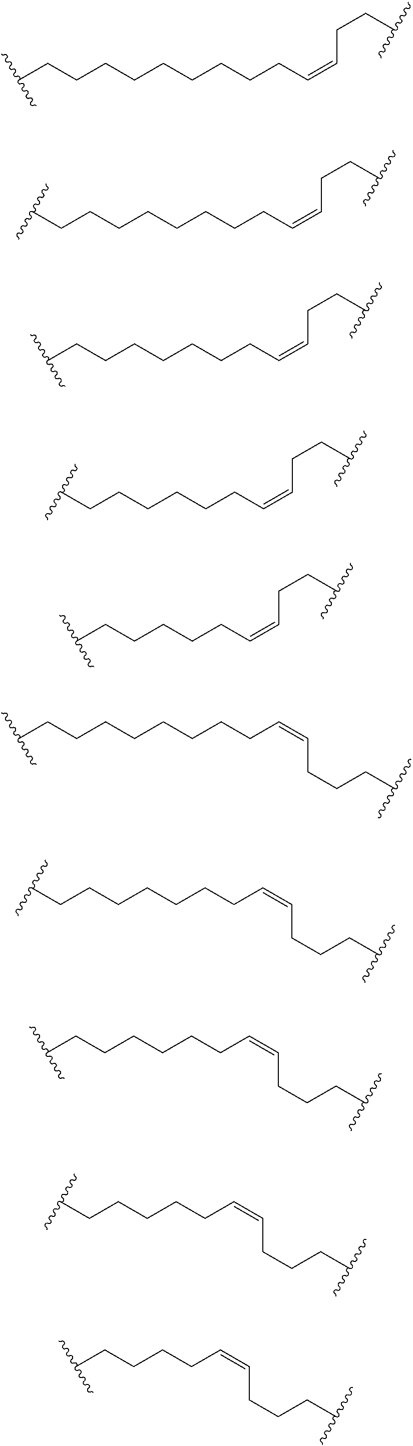

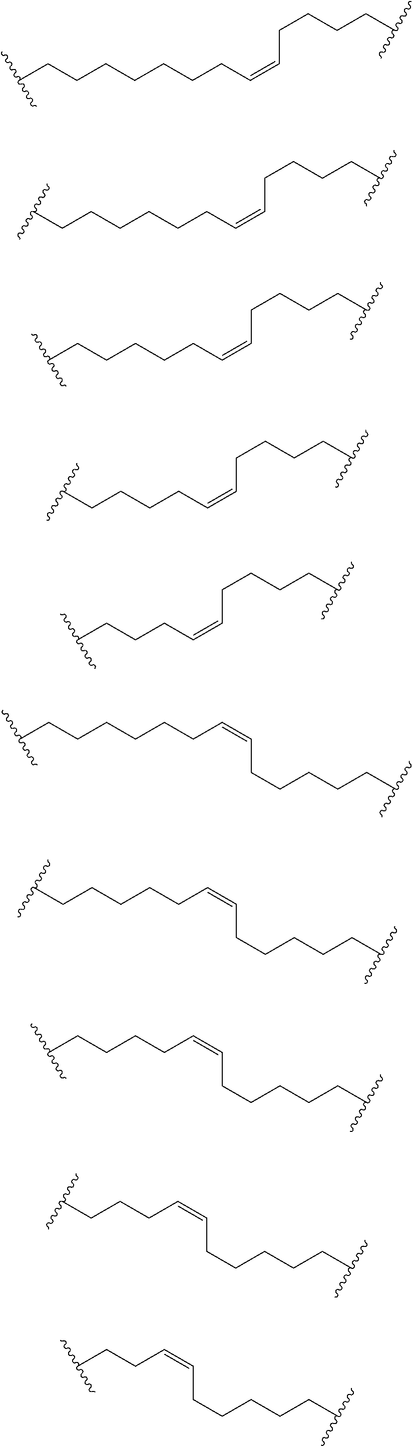

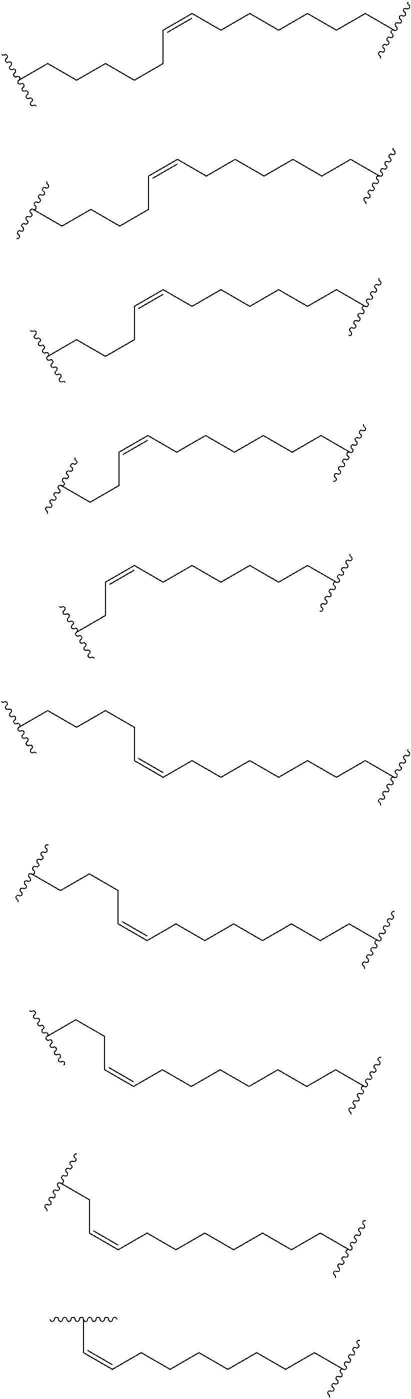

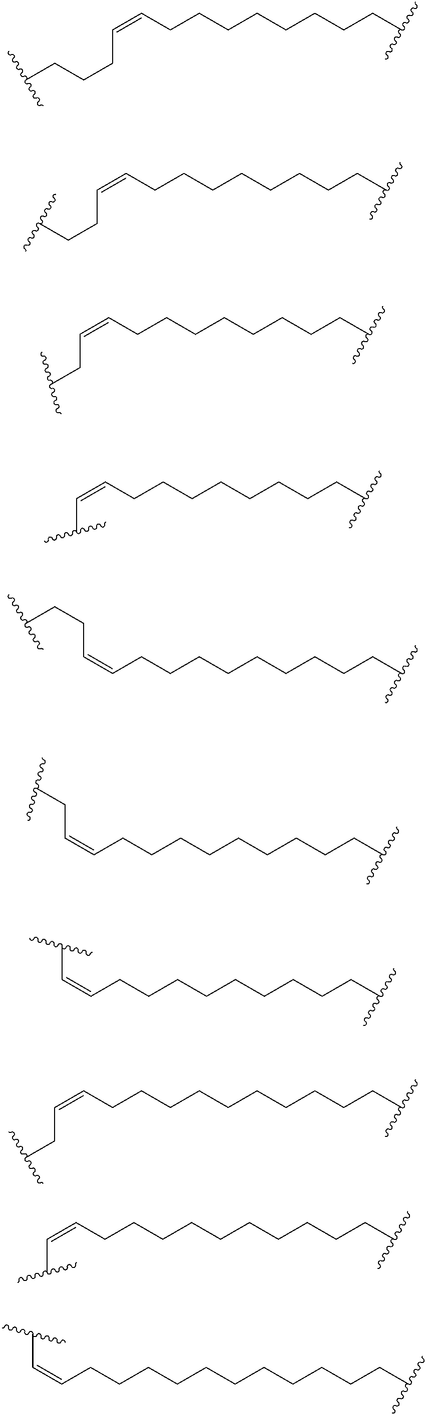

In some embodiments, L is selected from:

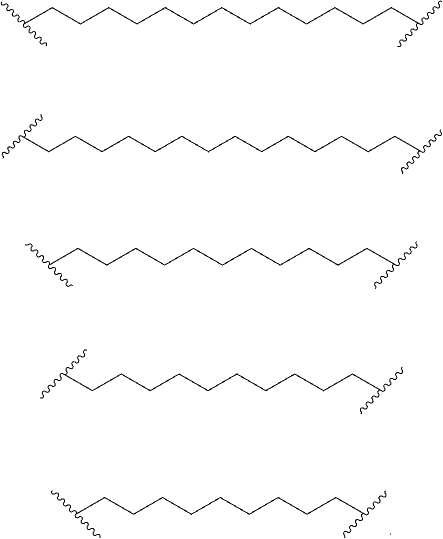 [SMILES: *CCCCCCCCCCCCC* ; *CCCCCCCCCCCCCC* ; *CCCCCCCCCCC* ; *CCCCCCCCCC* ; *CCCCCCCCC*]

In some embodiments, L is selected from:

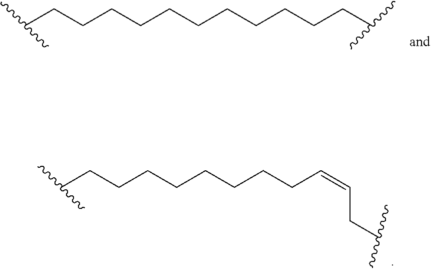 [SMILES: *CCCCCCCCCCC* ; *C/C=C\CCCCCCCC*]

As defined herein, R³ is optionally substituted aliphatic or optionally substituted heteroaliphatic. In some embodiments, R³ is optionally substituted aliphatic, —OR^{3O}, or —N(R^{3N})₂, wherein each instance of R^{3O} and R^{3N} is independently optionally substituted aliphatic. In certain embodiments, R³ is C₁-C₂₅ optionally substituted aliphatic, —OR^{3O}, or —N(R^{3N})₂, wherein each instance of R^{3O} and R^{3N} is independently optionally substituted C₁-C₂₅ aliphatic. In some embodiments, R³ is optionally substituted aliphatic or —OR^{3O}, wherein R^{3O} is optionally substituted aliphatic. In certain embodiments, R³ is C₁-C₂₅ optionally substituted aliphatic or —OR^{3O}, wherein R^{3O} is optionally substituted C₁-C₂₅ aliphatic.

In some embodiments, R³ is C₁-C₂₅ optionally substituted aliphatic. In some embodiments, R³ is C₁-C₂₅ substituted aliphatic. In certain embodiments, R³ is C₁-C₂₅ unsubstituted aliphatic. In some embodiments, R³ is C₁-C₂₅ optionally substituted alkyl. In some embodiments, R³ is C₁-C₂₅ substituted alkyl. In certain embodiments, R³ is C₁-C₂₅ unsubstituted alkyl. In some embodiments, R³ is C₁-C₂₅ alkyl substituted with C₃-C₈ unsubstituted cycloalkyl. In some embodiments, R³ is optionally substituted C₁-C₁₅ alkyl. In some embodiments, R³ is substituted C₁-C₁₅ alkyl. In some embodiments, R³ is unsubstituted C₁-C₁₅ alkyl. In some embodiments, R³ is C₁-C₁₅ alkyl substituted with C₃-C₈ unsubstituted cycloalkyl. In some embodiments, R³ is

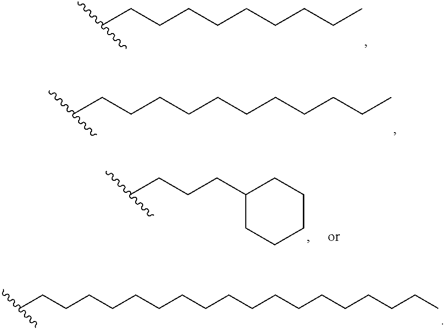

In some embodiments, R³ is

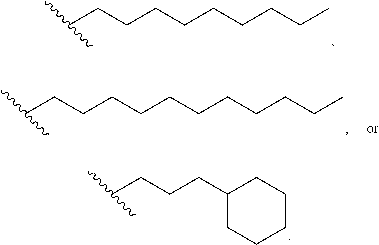

In some embodiments, R³ is

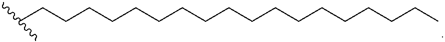 [SMILES: *CCCCCCCCCCCCCCCCCC]

In some embodiments, R³ is C₁-C₂₅ optionally substituted alkenyl. In some embodiments, R³ is C₁-C₂₅ substituted alkenyl. In certain embodiments, R³ is C₁-C₂₅ unsubstituted alkenyl. In some embodiments, R³ is C₁-C₂₅ optionally substituted alkenyl comprising one double bond. In some embodiments, R³ is C₁-C₂₅ optionally substituted alkenyl comprising at least two double bonds. In some embodiments, R³ is

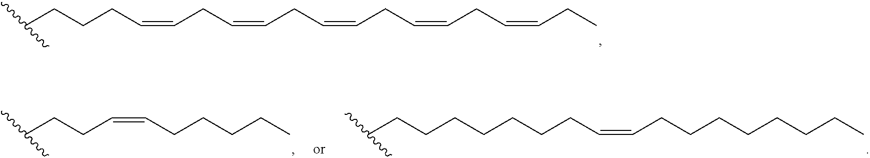

In some embodiments, R³ is

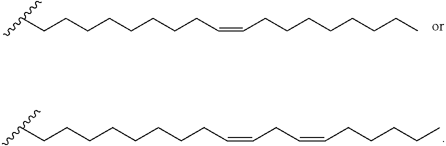 [SMILES: *CCCCCCCC/C=C\CCCCCCCC ; *CCCCCCCC/C=C\C/C=C\CCCCC]

In some embodiments, R³ is

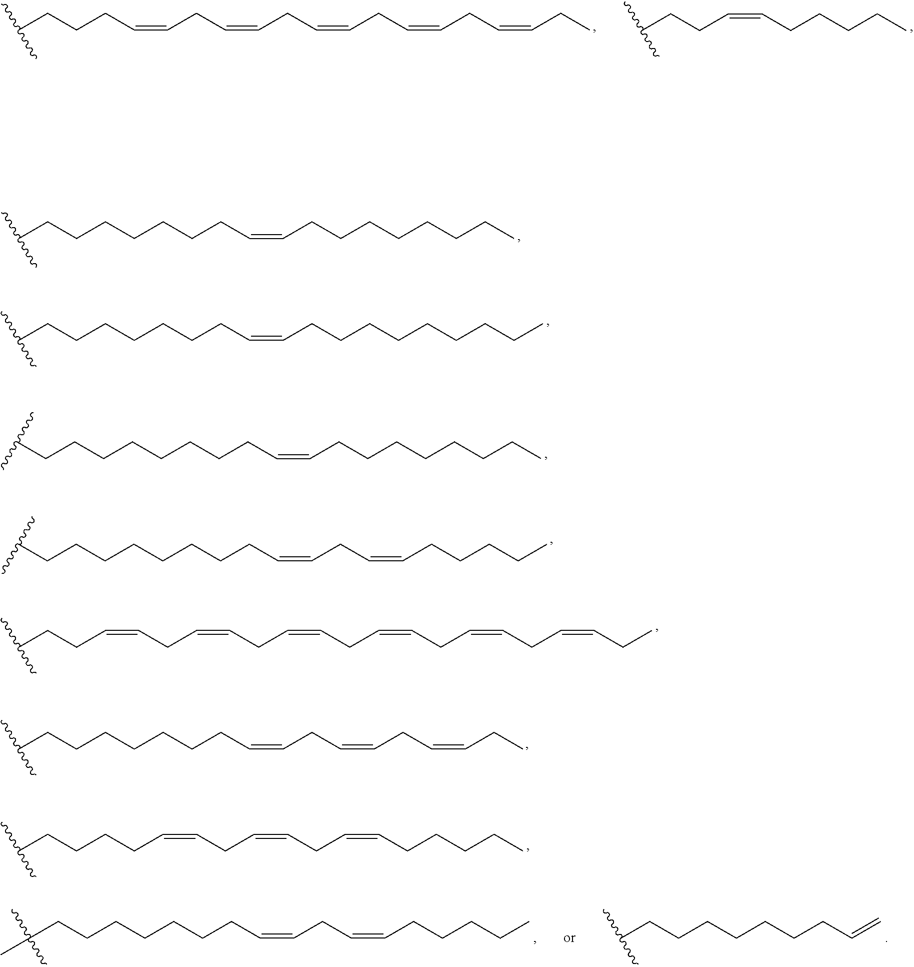

In some embodiments, R³ is C₁-C₂₅ optionally substituted alkynyl. In some embodiments, R³ is C₁-C₂₅ substituted alkynyl. In certain embodiments, R³ is C₁-C₂₅ unsubstituted alkynyl. In some embodiments, R³ is

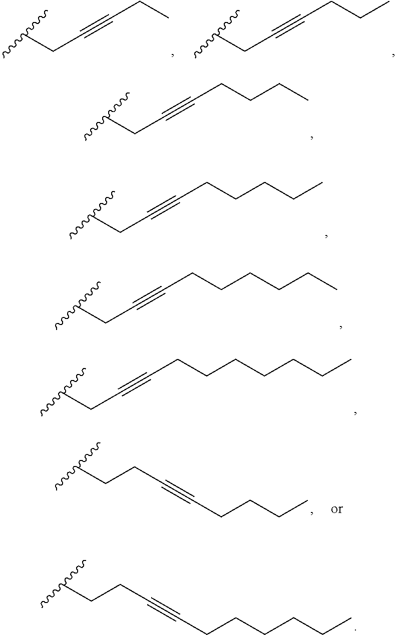

In certain embodiments, R³ is selected from the group consisting of

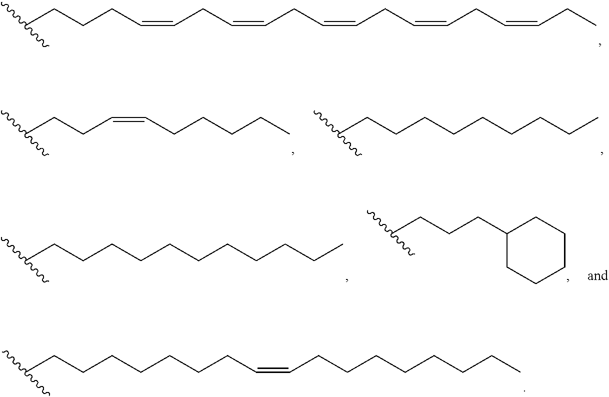

In certain embodiments, R³ is selected from the group consisting of

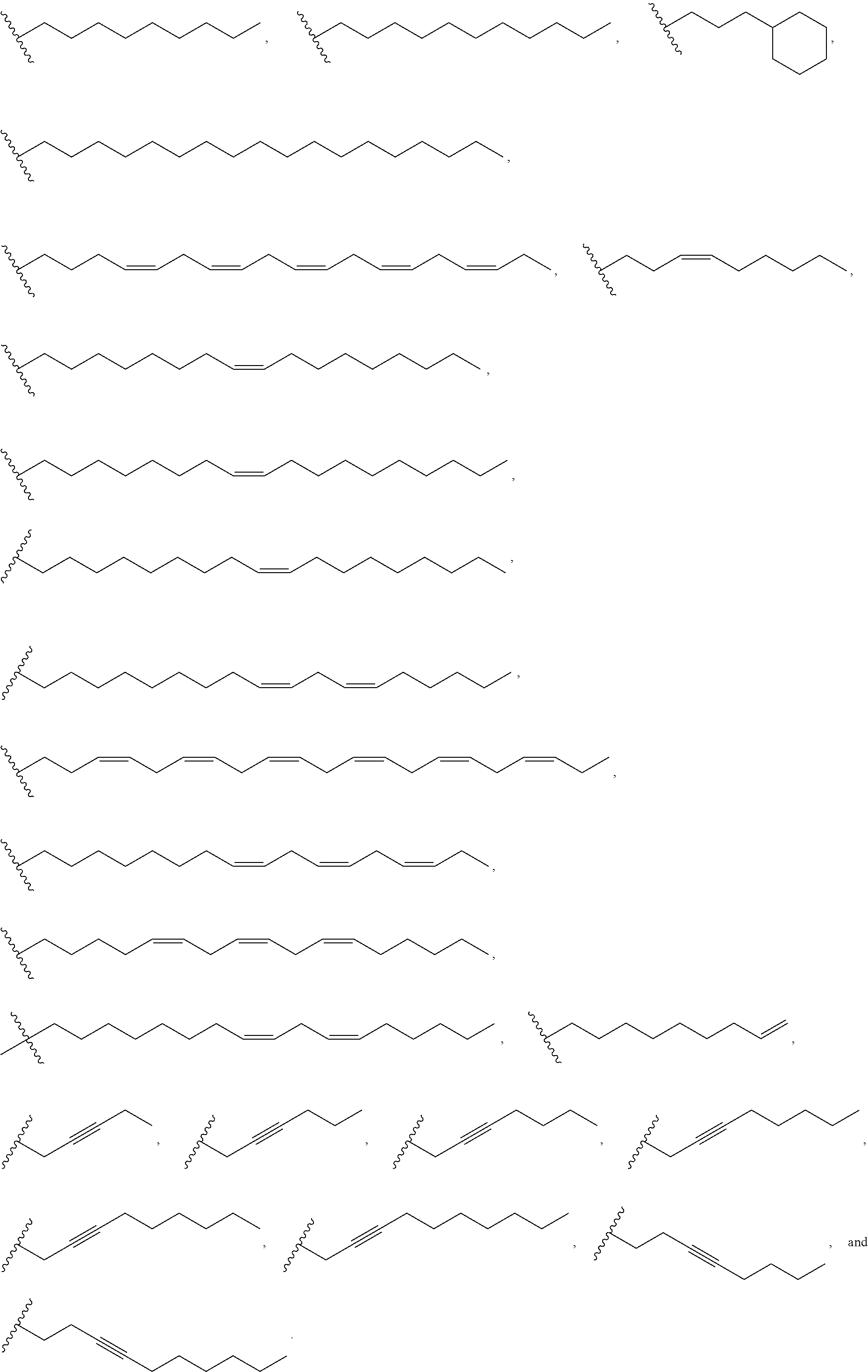

In certain embodiments, R³ is selected from the group consisting of

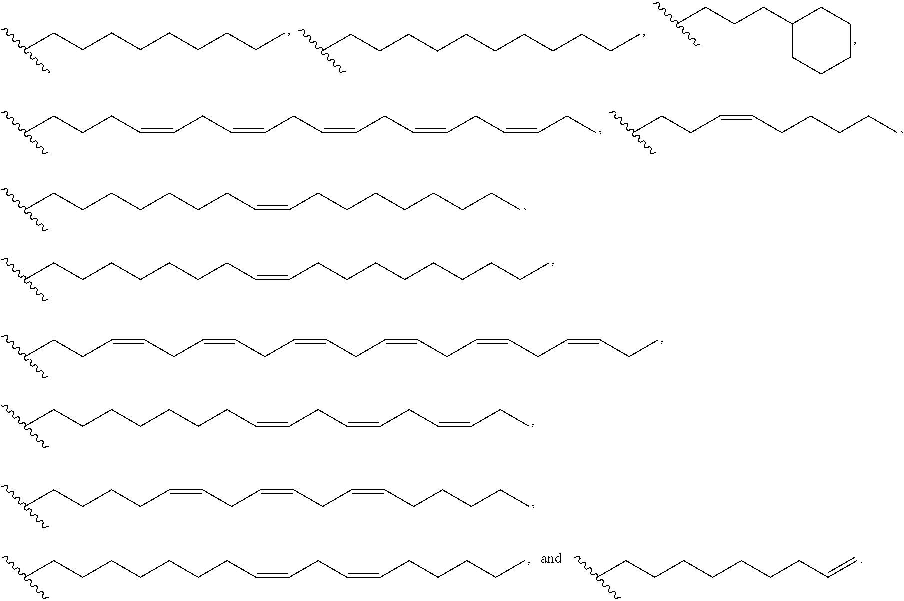

In some embodiments, R³ is optionally substituted C₁-C₂₅ heteroaliphatic. In certain embodiments, R³ is —OR^{3O} or —N(R^{3N})₂, wherein each instance of R^{3O} and R^{3N} is independently optionally substituted aliphatic. In certain embodiments, R³ is —OR^{3O} or —N(R^{3N})₂, wherein each instance of R^{3O} and R^{3N} is independently optionally substituted C₁-C₂₅ aliphatic.

In certain embodiments, R³ is —OR^{3O}, wherein R^{3O} is optionally substituted C₁-C₂₅ aliphatic.

In some embodiments, R^{3O} is C₁-C₂₅ optionally substituted aliphatic. In some embodiments, R^{3O} is C₁-C₂₅ substituted aliphatic. In certain embodiments, R^{3O} is C₁-C₂₅ unsubstituted aliphatic. In some embodiments, R^{3O} is C₁-C₂₅ optionally substituted alkyl. In some embodiments, R^{3O} is C₁-C₂₅ substituted alkyl. In certain embodiments, R^{3O} is C₁-C₂₅ unsubstituted alkyl. In some embodiments, R^{3O} is

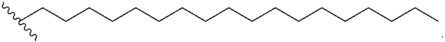 [SMILES: *CCCCCCCCCCCCCCCCCC]

In some embodiments, R^{3O} is C₁-C₂₅ optionally substituted alkenyl. In some embodiments, R^{3O} is C₁-C₂₅ substituted alkenyl. In certain embodiments, R^{3O} is C₁-C₂₅ unsubstituted alkenyl. In some embodiments, R^{3O} is C₁-C₂₅ optionally substituted alkenyl comprising one double bond. In some embodiments, R^{3O} is C₁-C₂₅ optionally substituted alkenyl comprising at least two double bonds. In some embodiments, R^{3O} is

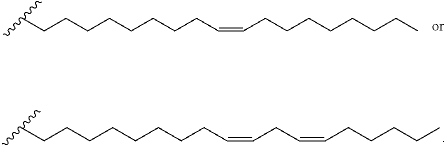 [SMILES: *CCCCCCCC/C=C\CCCCCCCC ; *CCCCCCCC/C=C\C/C=C\CCCCC]

In some embodiments, R^{3O} is C₁-C₂₅ optionally substituted alkynyl. In some embodiments, R^{3O} is C₁-C₂₅ substituted alkynyl. In certain embodiments, R^{3O} is C₁-C₂₅ unsubstituted alkynyl. In some embodiments, R^{3O} is

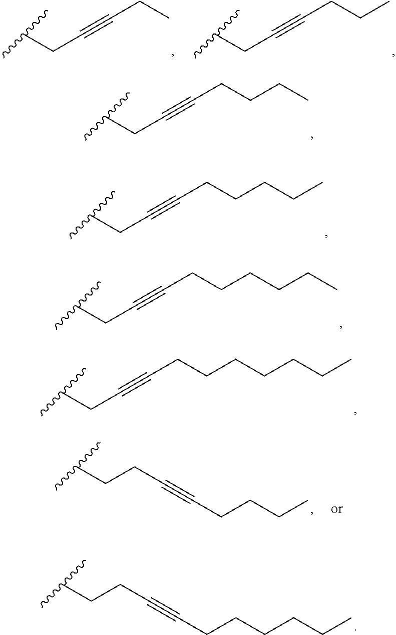

In some embodiments, R^{3O} is selected from the group consisting of

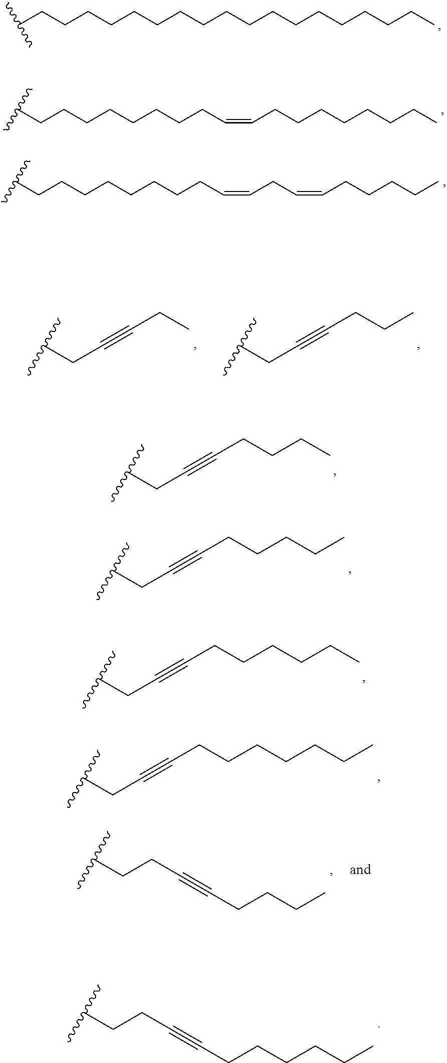

In certain embodiments, R³ is —N(R^{3N})₂, wherein each instance of R^{3N} is independently optionally substituted C₁-C₂₅ aliphatic.

In some embodiments, R^{3N} is C₁-C₂₅ optionally substituted aliphatic. In some embodiments, R^{3N} is C₁-C₂₅ substituted aliphatic. In certain embodiments, R^{3N} is C₁-C₂₅ unsubstituted aliphatic. In some embodiments, R^{3N} is C₁-C₂₅ optionally substituted alkyl. In some embodiments, R^{3N} is C₁-C₂₅ substituted alkyl.

In some embodiments, R^{3N} is C₁-C₂₅ optionally substituted alkenyl. In some embodiments, R^{3N} is C₁-C₂₅ substituted alkenyl. In certain embodiments, R^{3N} is C₁-C₂₅ unsubstituted alkenyl. In some embodiments, R^{3N} is C₁-C₂₅ optionally substituted alkenyl comprising one double bond. In some embodiments, R^{3N} is C₁-C₂₅ optionally substituted alkenyl comprising at least two double bonds.

In some embodiments, R^{3N} is C₁-C₂₅ optionally substituted alkynyl. In some embodiments, R^{3N} is C₁-C₂₅ substituted alkynyl. In certain embodiments, R^{3N} is C₁-C₂₅ unsubstituted alkynyl.

In some embodiments, the compound of Formula (I) is of Formula (II):

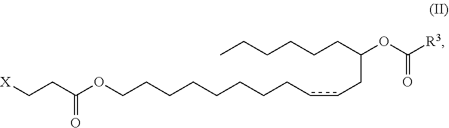

and pharmaceutically acceptable salts, solvates, tautomers, stereoisomers, or isotopically labeled derivatives thereof, wherein:
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl;
R³ is optionally substituted aliphatic or optionally substituted heteroaliphatic; and

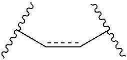

represents either a single or a double bond.

In certain embodiments, the compound of Formula (II) is of Formula (II-A):

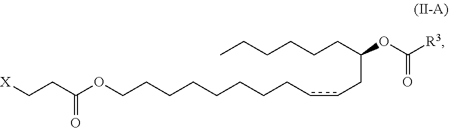

or a pharmaceutically acceptable salt, solvate, tautomer, stereoisomer, or isotopically labeled derivative thereof.

In certain embodiments, the compound of Formula (II) is of Formula (II-B):

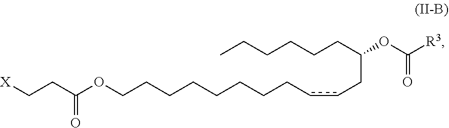

or a pharmaceutically acceptable salt, solvate, tautomer, stereoisomer, or isotopically labeled derivative thereof.

In certain embodiments, the compound of Formula (II) is of Formula (II-C):

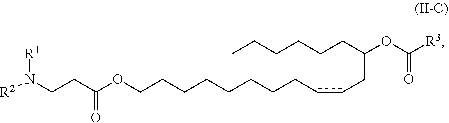

or a pharmaceutically acceptable salt, solvate, tautomer, stereoisomer, or isotopically labeled derivative thereof.

As defined herein, R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group; or R¹ and R² are joined together with the intervening atoms to form an optionally substituted heterocyclyl.

In certain embodiments, R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group. In some embodiments, R¹ is —H, optionally substituted alkyl, optionally substituted heteroalkyl, optionally substituted aryl, optionally substituted heteroaryl, optionally substituted cycloalkyl, or optionally substituted heterocyclyl. In certain embodiments, R¹ is optionally substituted alkyl, optionally substituted heteroalkyl, optionally substituted aryl, optionally substituted heteroaryl, optionally substituted cycloalkyl, or optionally substituted heterocyclyl. In some embodiments, R¹ is —H, optionally substituted alkyl, optionally substituted heteroalkyl, optionally substituted cycloalkyl, or optionally substituted heterocyclyl. In some embodiments, R¹ is optionally substituted alkyl, optionally substituted heteroalkyl, optionally substituted cycloalkyl, or optionally substituted heterocyclyl. In certain embodiments, R¹ is —H, optionally substituted alkyl, optionally substituted heteroalkyl, or optionally substituted heterocyclyl. In certain embodiments, R¹ is optionally substituted alkyl, optionally substituted heteroalkyl, or optionally substituted heterocyclyl.

In certain embodiments, R¹ is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R¹ is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R¹ is optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In some embodiments, R¹ is optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In certain embodiments, R¹ is —H, optionally substituted C₁-C₂₅ alkyl, optionally substituted C₁-C₂₅ heteroalkyl, or optionally substituted 3- to 8-membered heterocyclyl. In certain embodiments, R¹ is optionally substituted C₁-C₂₅ alkyl, optionally substituted C₁-C₂₅ heteroalkyl, or optionally substituted 3- to 8-membered heterocyclyl.

In certain embodiments, R¹ is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R¹ is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R¹ is optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In some embodiments, R¹ is optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In certain embodiments, R¹ is —H, optionally substituted C₁-C₂₀ alkyl, optionally substituted C₁-C₂₀ heteroalkyl, or optionally substituted 3- to 8-membered heterocyclyl. In certain embodiments, R¹ is optionally substituted C₁-C₂₀ alkyl, optionally substituted C₁-C₂₀ heteroalkyl, or optionally substituted 3- to 8-membered heterocyclyl.

In some embodiments, R¹ is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃₋₈ cycloalkyl, optionally substituted 3- to 8-membered heterocyclyl, or a protecting group. In some embodiments, R¹ is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-s cycloalkyl, or optionally substituted 3- to 8-membered heterocyclyl. In certain embodiments, R¹ is optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In some embodiments, R¹ is optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In certain embodiments, R¹ is —H, optionally substituted C₁-C₁₀ alkyl, optionally substituted C₁-C₁₀ heteroalkyl, or optionally substituted 3- to 8-membered heterocyclyl. In certain embodiments, R¹ is optionally substituted C₁-C₁₀ alkyl, optionally substituted C₁-C₁₀ heteroalkyl, or optionally substituted 3- to 8-membered heterocyclyl.

In some embodiments, R¹ is a protecting group. In some embodiments the protecting group is a nitrogen protecting group, oxygen protecting group, or sulfur protecting group. In some embodiments, the protecting group is a nitrogen protecting group. In some embodiments the protecting group is an oxygen protecting group. In some embodiments, the protecting group is a sulfur protecting group. In some embodiments, R¹ is —H. In certain embodiments, R¹ is —H or a protecting group. In some embodiments, R¹ is —H or a nitrogen protecting group, an oxygen protecting group, or a sulfur protecting group. In some embodiments, R¹ is —H or a nitrogen protecting group.

In certain embodiments, R¹ is optionally substituted alkyl. In some embodiments, R¹ is optionally substituted C₁-C₂₅ alkyl. In certain embodiments, R¹ is optionally substituted C₁-C₂₀ alkyl. In some embodiments, R¹ is optionally substituted C₁-C₁₅ alkyl. In certain embodiments, R¹ is optionally substituted C₁-C₁₀ alkyl. In some embodiments, R¹ is optionally substituted C₁-C₆ alkyl. In certain embodiments, R¹ is substituted alkyl. In some embodiments, R¹ is substituted C₁-C₂₅ alkyl. In certain embodiments, R¹ is substituted C₁-C₂₀ alkyl. In some embodiments, R¹ is substituted C₁-C₁₅ alkyl. In certain embodiments, R¹ is substituted C₁-C₁₀ alkyl. In some embodiments, R¹ is substituted C₁-C₆ alkyl. In certain embodiments, R¹ is unsubstituted alkyl. In some embodiments, R¹ is unsubstituted C₁-C₂₅ alkyl. In certain embodiments, R¹ is unsubstituted C₁-C₂₀ alkyl. In some embodiments, R¹ is unsubstituted C₁-C₁₅ alkyl. In certain embodiments, R¹ is unsubstituted C₁-C₁₀ alkyl. In some embodiments, R¹ is unsubstituted C₁-C₆ alkyl. In certain embodiments, R¹ is methyl, ethyl, propyl, or butyl. In some embodiments, R¹ is methyl or ethyl. In certain embodiments, R¹ is methyl. In some embodiments, R¹ is ethyl.

In certain embodiments, R¹ is optionally substituted heteroalkyl. In some embodiments, R¹ is optionally substituted C₁-C₂₅ heteroalkyl. In certain embodiments, R¹ is optionally substituted C₁-C₂₀ heteroalkyl. In some embodiments, R¹ is optionally substituted C₁-C₁₅ heteroalkyl. In certain embodiments, R¹ is optionally substituted C₁-C₁₀ heteroalkyl. In some embodiments, R¹ is optionally substituted C₁-C₆ heteroalkyl. In certain embodiments, R¹ is substituted heteroalkyl. In some embodiments, R¹ is substituted C₁-C₂₅ heteroalkyl. In certain embodiments, R¹ is substituted C₁-C₂₀ heteroalkyl. In some embodiments, R¹ is substituted C₁-C₁₅ heteroalkyl. In certain embodiments, R¹ is substituted C₁-C₁₀ heteroalkyl. In some embodiments, R¹ is substituted C₁-C₆ heteroalkyl. In certain embodiments, R¹ is unsubstituted heteroalkyl. In some embodiments, R¹ is unsubstituted C₁-C₂₅ heteroalkyl. In certain embodiments, R¹ is unsubstituted C₁-C₂₀ heteroalkyl. In some embodiments, R¹ is unsubstituted C₁-C₁₅ heteroalkyl. In certain embodiments, R¹ is unsubstituted C₁-C₁₀ heteroalkyl. In some embodiments, R¹ is unsubstituted C₁-C₆ heteroalkyl. In some embodiments, R¹ is

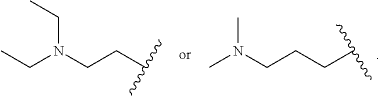

In some embodiments, R¹ is optionally substituted C₆-C₁₄ aryl. In some embodiments, R¹ is substituted C₆-C₁₄ aryl. In some embodiments, R¹ is unsubstituted C₆-C₁₄ aryl. In certain embodiments, R¹ is optionally substituted C₆-C₁₀ aryl. In certain embodiments, R¹ is substituted C₆-C₁₀ aryl. In certain embodiments, R¹ is unsubstituted C₆-C₁₀ aryl. In some embodiments, R¹ is optionally substituted phenyl or optionally substituted naphthyl. In certain embodiments, R¹ is optionally substituted phenyl. In some embodiments, R¹ is optionally substituted naphthyl.

In some embodiments, R¹ is optionally substituted 5- to 14-membered heteroaryl. In some embodiments, R¹ is substituted 5- to 14-membered heteroaryl. In some embodiments, R¹ is unsubstituted 5- to 14-membered heteroaryl. In some embodiments, R¹ is optionally substituted 5- to 10-membered heteroaryl. In some embodiments, R¹ is substituted 5- to 10-membered heteroaryl. In some embodiments, R¹ is unsubstituted 5- to 10-membered heteroaryl. In some embodiments, R¹ is optionally substituted pyridinyl.

In some embodiments, R¹ is optionally substituted C₃-C₈ cycloalkyl. In some embodiments, R¹ is substituted C₃-C₈ cycloalkyl. In some embodiments, R¹ is unsubstituted C₃-C₈ cycloalkyl. In some embodiments, R¹ is C₃-C₈ cycloalkyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R¹ is optionally substituted 3- to 8-membered heterocyclyl. In some embodiments, R¹ is substituted 3- to 8-membered heterocyclyl. In some embodiments, R¹ is unsubstituted 3- to 8-membered heterocyclyl. In some embodiments, R¹ is 3- to 8-membered heterocyclyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R¹ is not

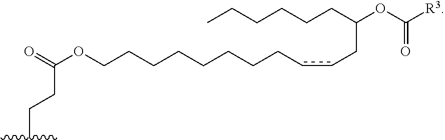

In certain embodiments, the compound of Formula (II) is of Formula (II-C-i):

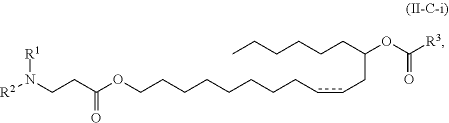

or a pharmaceutically acceptable salt, solvate, tautomer, stereoisomer, or isotopically labeled derivative thereof, wherein:

R¹ is —H, -Me, -Et, or

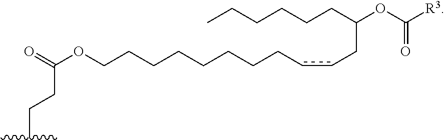

In certain embodiments, the compound of Formula (II) is of Formula (II-C-ii):

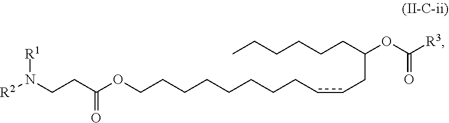

or a pharmaceutically acceptable salt, solvate, tautomer, stereoisomer, or isotopically labeled derivative thereof, wherein:
R¹ is —H, optionally substituted alkyl, optionally substituted heteroalkyl, optionally substituted aryl, optionally substituted heteroaryl, optionally substituted cycloalkyl, or optionally substituted heterocyclyl;
provided that R¹ is not:

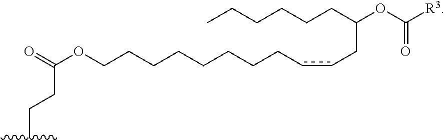

In certain embodiments R¹ is H, -Me, or -Et. In some embodiments, R¹ is —H, -Me, -Et,

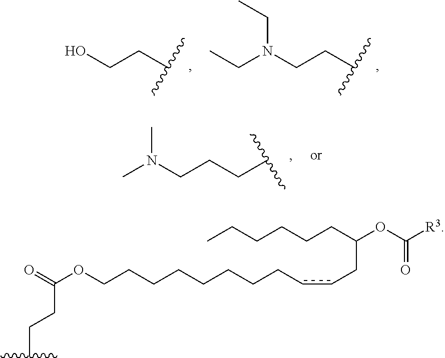

In some embodiments, R¹ is —H, -Me, -Et,

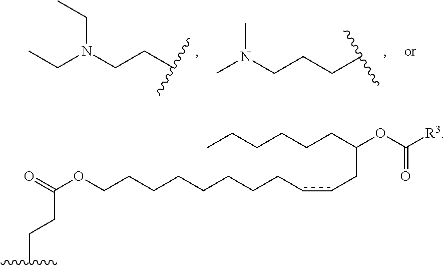

In some embodiments, R¹ is —H, -Me, -Et, or

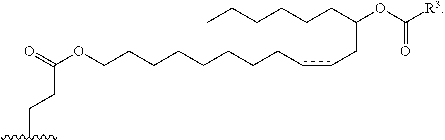

In some embodiments, R¹ is —H, -Me, -Et,

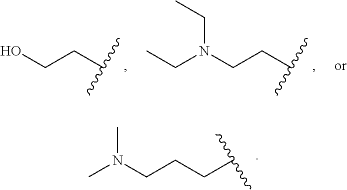

In some embodiments, R¹ is

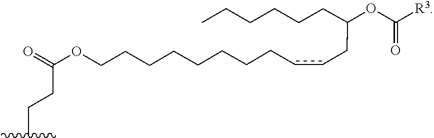

In certain embodiments, the compound of Formula (II) is of Formula (II-D):

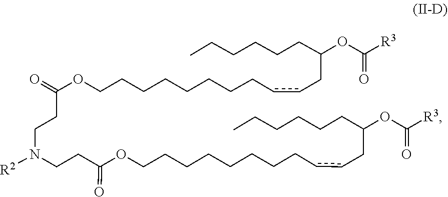

or a pharmaceutically acceptable salt, solvate, tautomer, stereoisomer, or isotopically labeled derivative thereof.

As defined herein, R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or R¹ and R² are joined together with the intervening atoms to form an optionally substituted heterocyclyl.

In certain embodiments, R² is —H, optionally substituted alkyl, optionally substituted heteroalkyl, optionally substituted aryl, optionally substituted heteroaryl, optionally substituted cycloalkyl, optionally substituted heterocyclyl, or a protecting group. In some embodiments, R² is —H, optionally substituted alkyl, optionally substituted heteroalkyl, optionally substituted aryl, optionally substituted heteroaryl, optionally substituted cycloalkyl, or optionally substituted heterocyclyl. In certain embodiments, R² is optionally substituted alkyl, optionally substituted heteroalkyl, optionally substituted aryl, optionally substituted heteroaryl, optionally substituted cycloalkyl, or optionally substituted heterocyclyl. In some embodiments, R² is —H, optionally substituted alkyl, optionally substituted heteroalkyl, optionally substituted cycloalkyl, or optionally substituted heterocyclyl. In some embodiments, R² is optionally substituted alkyl, optionally substituted heteroalkyl, optionally substituted cycloalkyl, or optionally substituted heterocyclyl. In certain embodiments, R² is —H, optionally substituted alkyl, optionally substituted heteroalkyl, or optionally substituted heterocyclyl. In certain embodiments, R² is optionally substituted alkyl, optionally substituted heteroalkyl, or optionally substituted heterocyclyl.

In certain embodiments, R² is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R² is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R² is optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In some embodiments, R² is optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In certain embodiments, R² is —H, optionally substituted C₁-C₂₅ alkyl, optionally substituted C₁-C₂₅ heteroalkyl, or optionally substituted 3- to 8-membered heterocyclyl. In certain embodiments, R² is optionally substituted C₁-C₂₅ alkyl, optionally substituted C₁-C₂₅ heteroalkyl, or optionally substituted 3- to 8-membered heterocyclyl.

In certain embodiments, R² is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R² is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R² is optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In some embodiments, R² is optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In certain embodiments, R² is —H, optionally substituted C₁-C₂₀ alkyl, optionally substituted C₁-C₂₀ heteroalkyl, or optionally substituted 3- to 8-membered heterocyclyl. In certain embodiments, R² is optionally substituted C₁-C₂₀ alkyl, optionally substituted C₁-C₂₀ heteroalkyl, or optionally substituted 3- to 8-membered heterocyclyl.

In some embodiments, R² is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃₋₈ cycloalkyl, optionally substituted 3- to 8-membered heterocyclyl, or a protecting group. In some embodiments, R² is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-s cycloalkyl, or optionally substituted 3- to 8-membered heterocyclyl. In certain embodiments, R² is optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In some embodiments, R² is optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In certain embodiments, R² is —H, optionally substituted C₁-C₁₀ alkyl, optionally substituted C₁-C₁₀ heteroalkyl, or optionally substituted 3- to 8-membered heterocyclyl. In certain embodiments, R² is optionally substituted C₁-C₁₀ alkyl, optionally substituted C₁-C₁₀ heteroalkyl, or optionally substituted 3- to 8-membered heterocyclyl.

In some embodiments, R² is a protecting group. In some embodiments the protecting group is a nitrogen protecting group. In some embodiments, R² is —H. In some embodiments, R² is —H or a protecting group. In some embodiments, R² is —H or a nitrogen protecting group.

In certain embodiments, R² is optionally substituted alkyl. In some embodiments, R² is optionally substituted C₁-C₂₅ alkyl. In certain embodiments, R² is optionally substituted C₁-C₂₀ alkyl. In some embodiments, R² is optionally substituted C₁-C₁₅ alkyl. In certain embodiments, R² is optionally substituted C₁-C₁₀ alkyl. In some embodiments, R² is optionally substituted C₁-C₆ alkyl. In certain embodiments, R² is substituted alkyl. In some embodiments, R² is substituted C₁-C₂₅ alkyl. In certain embodiments, R² is substituted C₁-C₂₀ alkyl. In some embodiments, R² is substituted C₁-C₁₅ alkyl. In certain embodiments, R² is substituted C₁-C₁₀ alkyl. In some embodiments, R² is substituted C₁-C₆ alkyl. In certain embodiments, R² is unsubstituted alkyl. In some embodiments, R² is unsubstituted C₁-C₂₅ alkyl. In certain embodiments, R² is unsubstituted C₁-C₂₀ alkyl. In some embodiments, R² is unsubstituted C₁-C₁₅ alkyl. In certain embodiments, R² is unsubstituted C₁-C₁₀ alkyl. In some embodiments, R² is unsubstituted C₁-C₆ alkyl. In certain embodiments, R² is methyl, ethyl, propyl, or butyl. In some embodiments, R² is methyl or ethyl. In certain embodiments, R² is methyl. In some embodiments, R² is ethyl.

In certain embodiments, R² is optionally substituted heteroalkyl. In some embodiments, R² is optionally substituted C₁-C₂₅ heteroalkyl. In certain embodiments, R² is optionally substituted C₁-C₂₀ heteroalkyl. In some embodiments, R² is optionally substituted C₁-C₁₅ heteroalkyl. In certain embodiments, R² is optionally substituted C₁-C₁₀ heteroalkyl. In some embodiments, R² is optionally substituted C₁-C₆ heteroalkyl. In certain embodiments, R² is substituted heteroalkyl. In some embodiments, R² is substituted C₁-C₂₅ heteroalkyl. In certain embodiments, R² is substituted C₁-C₂₀ heteroalkyl. In some embodiments, R² is substituted C₁-C₁₅ heteroalkyl. In certain embodiments, R² is substituted C₁-C₁₀ heteroalkyl. In some embodiments, R² is substituted C₁-C₆ heteroalkyl. In certain embodiments, R² is unsubstituted heteroalkyl. In some embodiments, R² is unsubstituted C₁-C₂₅ heteroalkyl. In certain embodiments, R² is unsubstituted C₁-C₂₀ heteroalkyl. In some embodiments, R² is unsubstituted C₁-C₁₅ heteroalkyl. In certain embodiments, R² is unsubstituted C₁-C₁₀ heteroalkyl. In some embodiments, R² is unsubstituted C₁-C₆ heteroalkyl.

In certain embodiments, R² is optionally substituted heteroalkyl comprising one or more N atoms substituted with

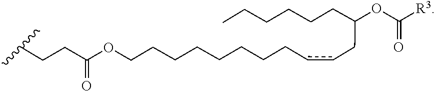

In some embodiments, R² is optionally substituted heteroalkyl, wherein the optionally substituted alkyl comprises two N atoms substituted with

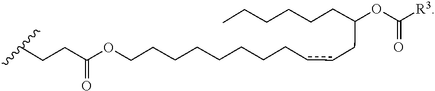

In certain embodiments, R² is optionally substituted heteroalkyl, wherein the optionally substituted alkyl comprises three N atoms substituted with

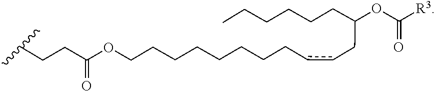

In certain embodiments,

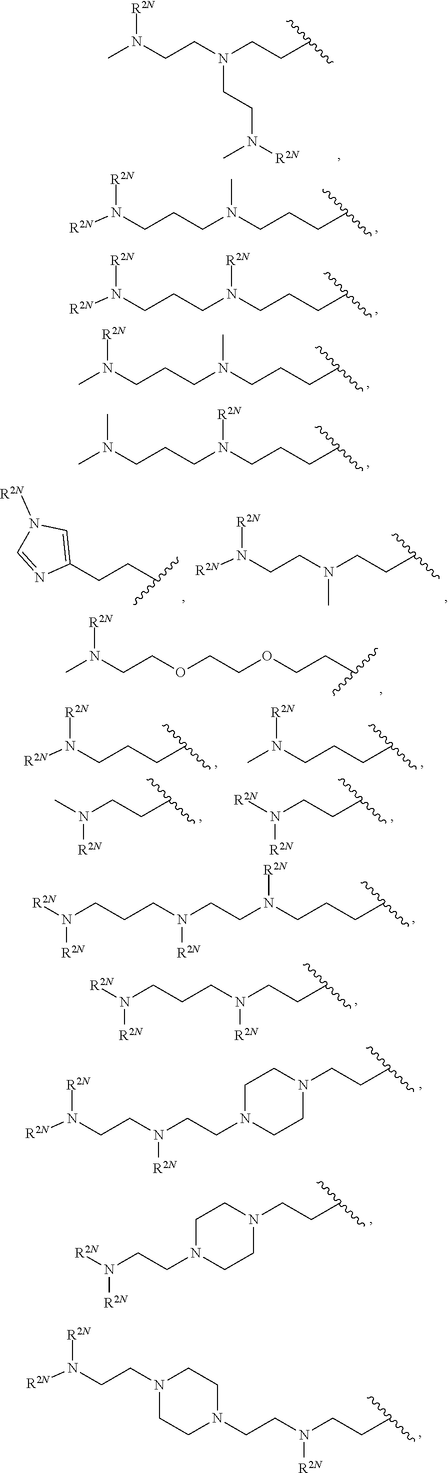

wherein each instance of R^{2N} is independently —H or

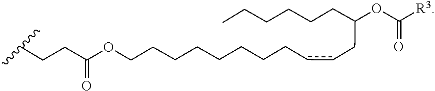

In certain embodiments, R² is

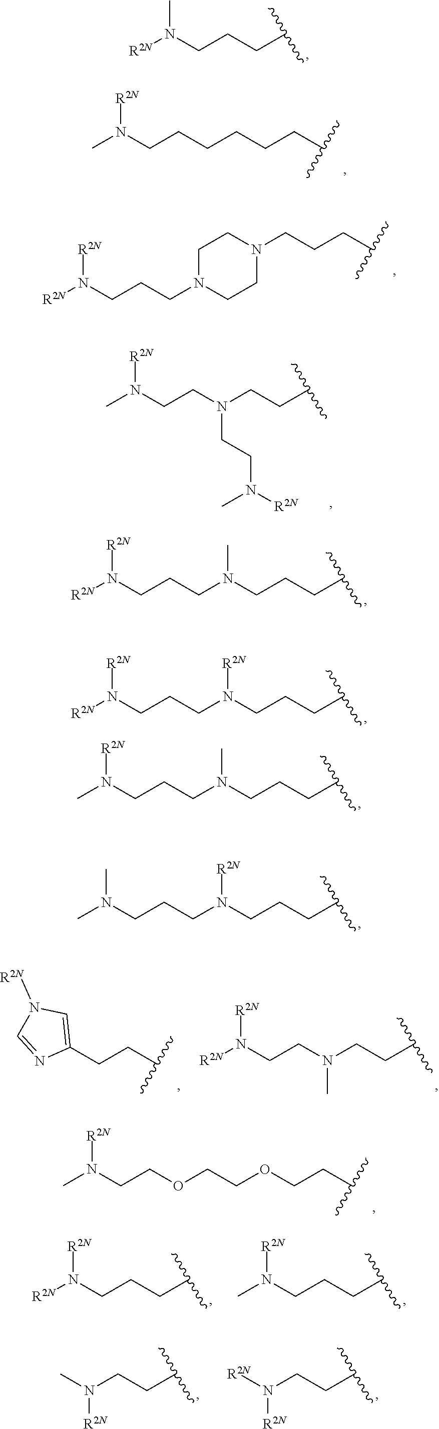

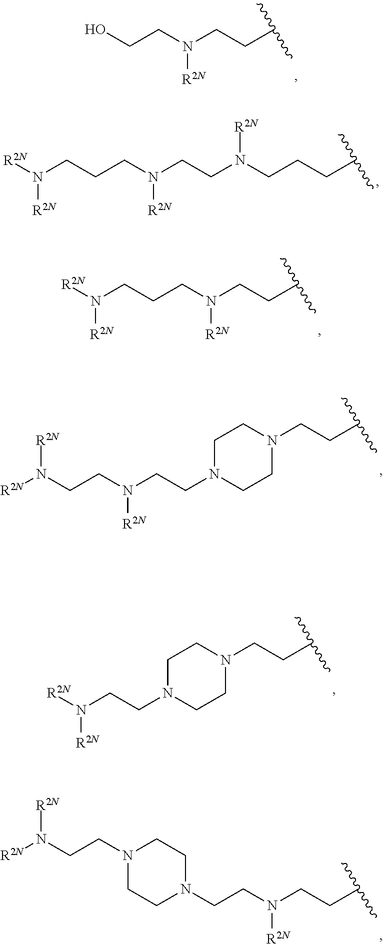

wherein each instance of R^{2N} is independently —H, optionally substituted C₁₋₁₀ aliphatic, optionally substituted C₁₋₁₀ heteroaliphatic, optionally substituted acyl, a nitrogen protecting group, or

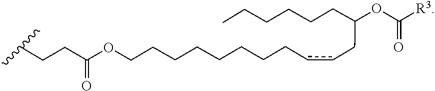

In certain embodiments, R² is

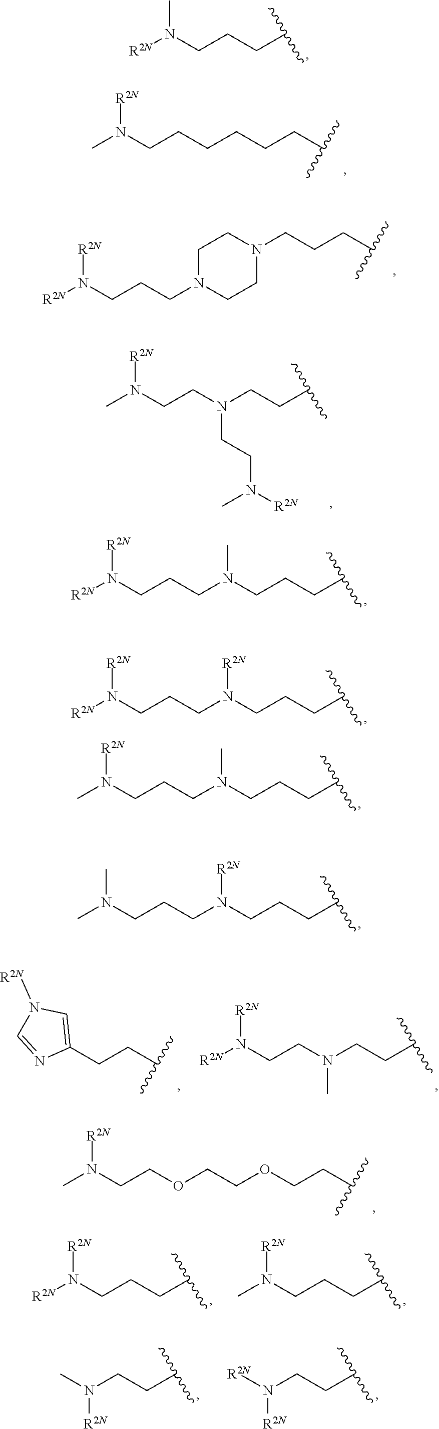

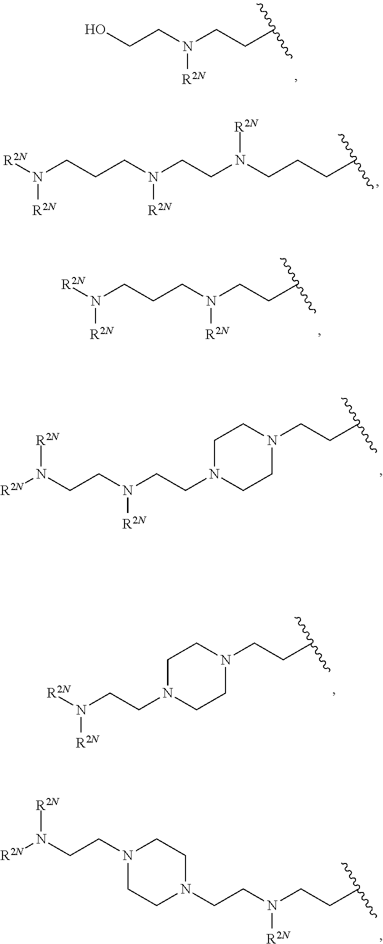

wherein each instance of R^{2N} is independently —H or

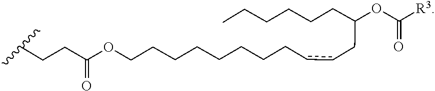

In some embodiments, R^{2N} is —H or

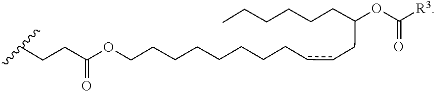

In some embodiments, R^{2N} is —H. In certain embodiments, R^{2N} is

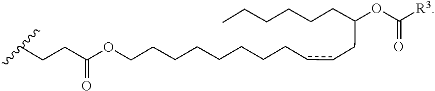

In some embodiments, R^{2N} is optionally substituted acyl. In some embodiments, R^{2N} is a nitrogen protecting group. In some embodiments, R^{2N} is Bn, Boc, Cbz, Fmoc, trifluoroacetyl, triphenylmethyl, acetyl, or Ts. In some embodiments, R^{2N} is optionally substituted C₁₋₁₀ aliphatic or optionally substituted C₁₋₁₀ heteroaliphatic. In some embodiments, R^{2N} is optionally substituted C₁₋₁₀ aliphatic. In some embodiments, R^{2N} is C₁₋₁₀ alkyl. In some embodiments, R^{2N} is -Me, -Et, -nPr, -iPr, -nBu, -iBu, or -tBu. In some embodiments, R^{2N} is -Me or -Et. In some embodiments, R^{2N} is -Me. In some embodiments, R^{2N} is -Et. In some embodiments, R^{2N} is C₂₋₁₀ alkenyl. In some embodiments, R^{2N} is C₂₋₁₀ alkynyl. In some embodiments, R^{2N} is optionally substituted C₁₋₁₀ heteroaliphatic. In some embodiments, R^{2N} is C₁₋₁₀ heteroalkyl. In some embodiments, R^{2N} is C₂₋₁₀ heteroalkenyl. In some embodiments, R^{2N} is C₂₋₁₀ heteroalkynyl.

In some embodiments, R² is optionally substituted C₆-C₁₄ aryl. In some embodiments, R² is substituted C₆-C₁₄ aryl. In some embodiments, R² is unsubstituted C₆-C₁₄ aryl. In certain embodiments, R² is optionally substituted C₆-C₁₀ aryl. In certain embodiments, R² is substituted C₆-C₁₀ aryl. In certain embodiments, R² is unsubstituted C₆-C₁₀ aryl. In some embodiments, R² is optionally substituted phenyl or optionally substituted naphthyl. In certain embodiments, R² is optionally substituted phenyl. In some embodiments, R² is optionally substituted naphthyl.

In some embodiments, R² is optionally substituted 5- to 14-membered heteroaryl. In some embodiments, R² is substituted 5- to 14-membered heteroaryl. In some embodiments, R² is unsubstituted 5- to 14-membered heteroaryl. In some embodiments, R² is optionally substituted 5- to 10-membered heteroaryl. In some embodiments, R² is substituted 5- to 10-membered heteroaryl. In some embodiments, R² is unsubstituted 5- to 10-membered heteroaryl. In some embodiments, R² is optionally substituted pyridinyl.

In some embodiments, R² is optionally substituted C₃-C₈ cycloalkyl. In some embodiments, R² is substituted C₃-C₈ cycloalkyl. In some embodiments, R² is unsubstituted C₃-C₈ cycloalkyl. In some embodiments, R² is C₃-C₈ cycloalkyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R² is optionally substituted 3- to 8-membered heterocyclyl. In some embodiments, R² is substituted 3- to 8-membered heterocyclyl. In some embodiments, R² is unsubstituted 3- to 8-membered heterocyclyl. In some embodiments, R² is 3- to 8-membered heterocyclyl substituted with optionally substituted alkyl, optionally substituted heteroalkyl, optionally substituted cycloalkyl, optionally substituted heterocyclyl, optionally substituted heteroaryl, or optionally substituted aryl.

In certain embodiments, R² is selected from the group consisting of -Me,

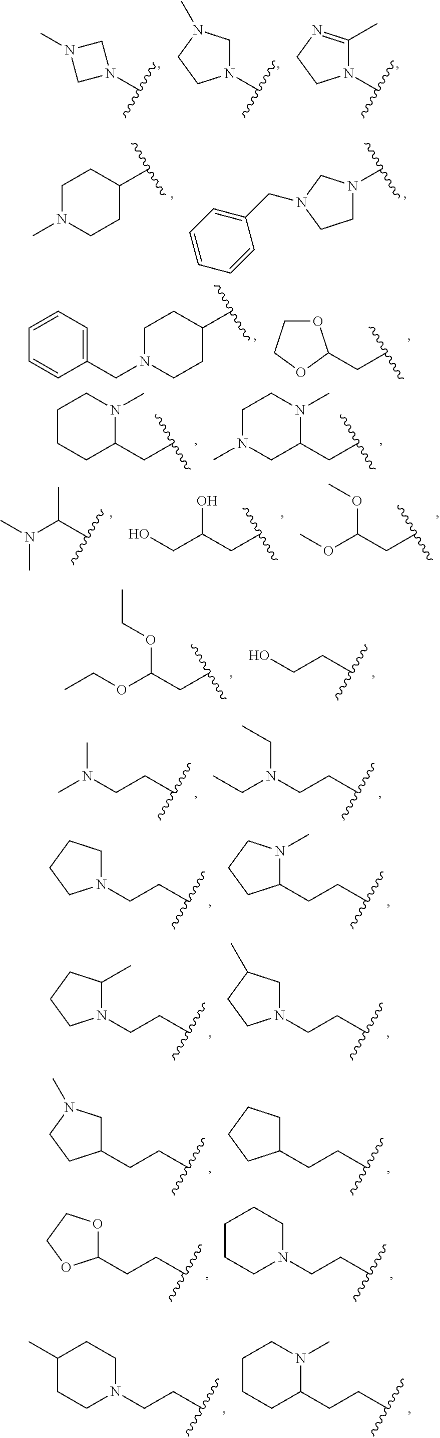

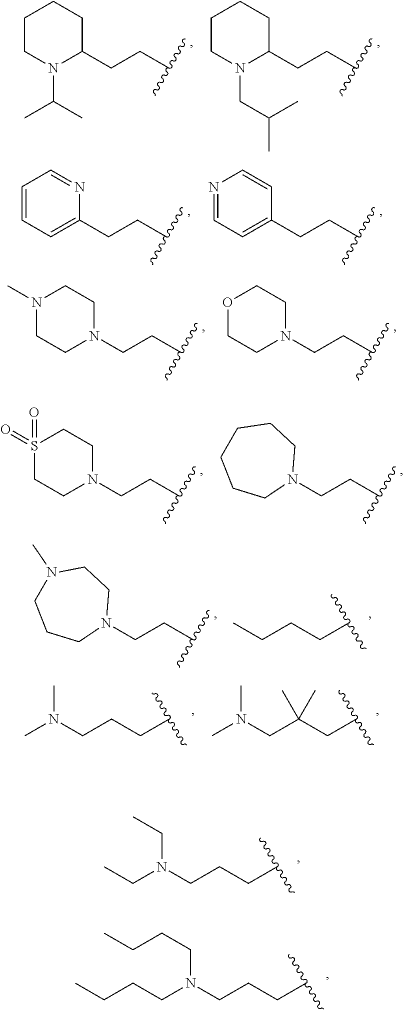

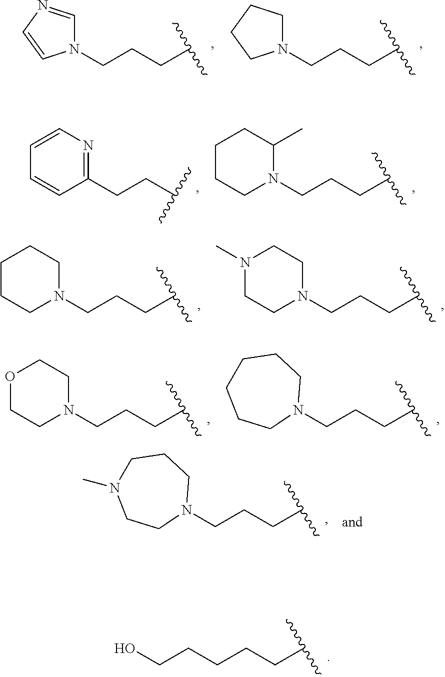

In certain embodiments, R² is selected from the group consisting of -Me,

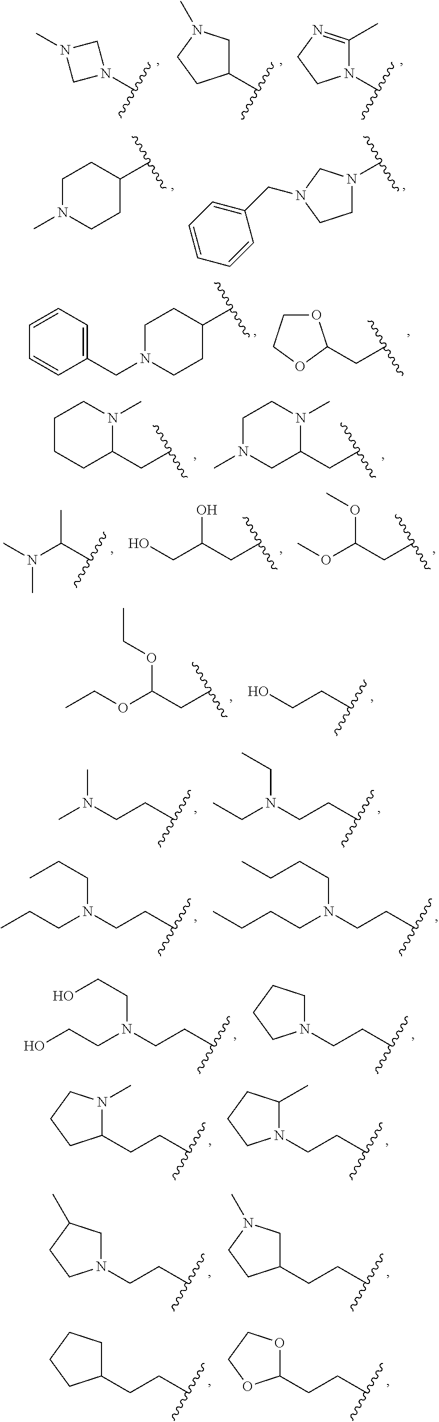

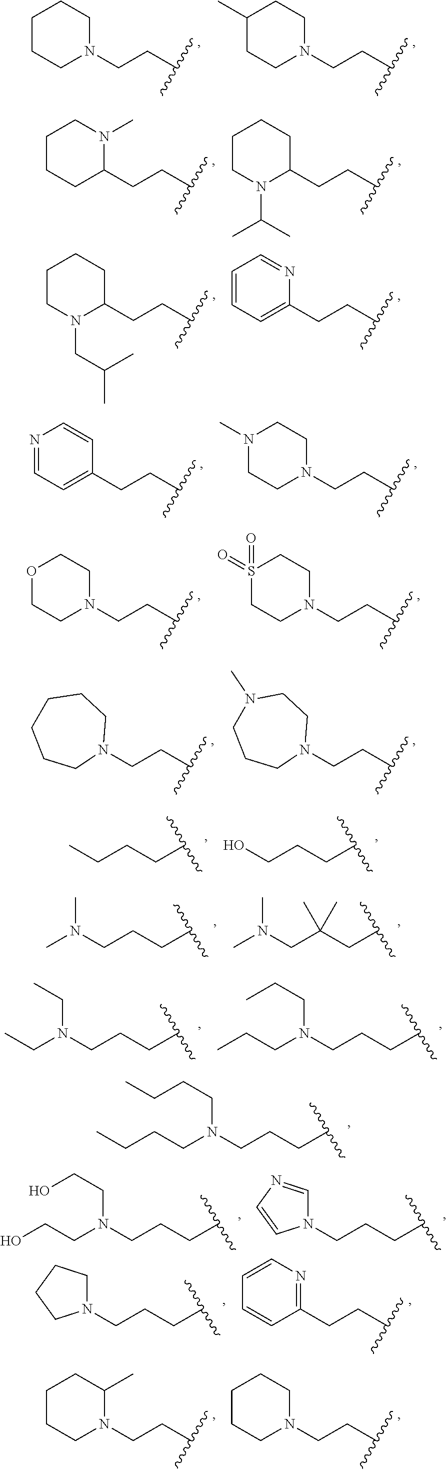

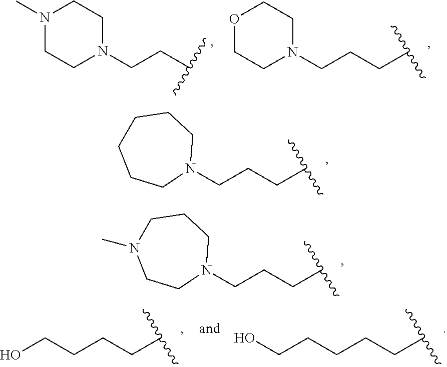

In some embodiments, R¹ is not —H. In certain embodiments, R² is not —H. In some embodiments, R¹ and R² are not both H.

In certain embodiments, R¹ and R² are joined together with the intervening atoms to form an optionally substituted heterocycle. In some embodiments, R¹ and R² are joined together with the intervening atoms to form an optionally substituted N-heterocycle. In certain embodiments, R¹ and R² are joined together with the intervening atoms to form an optionally substituted 3- to 6-membered heterocycle. In some embodiments, R¹ and R² are joined together with the intervening atoms to form an optionally substituted 3- to 6-membered N-heterocycle. In some embodiments, R¹ and R² are joined together with the intervening atoms to form a substituted 3- to 6-membered N-heterocycle. In some embodiments, R¹ and R² are joined together with the intervening atoms to form an unsubstituted 3- to 6-membered N-heterocycle. In certain embodiments, R¹ and R² are joined together with the intervening atoms to form optionally substituted aziridine, azetidine, pyrrolidine, piperidine, or piperazine. In certain embodiments, R¹ and R² are joined together with the intervening atoms to form aziridine, azetidine, pyrrolidine, piperidine, or piperazine substituted with C₁-C₁₀ alkyl, C₁-C₁₀ heteroalkyl, C₃-C₈ carbocyclyl, 3- to 8-membered heterocyclyl, C₆-C₁₄ aryl, or 5- to 14-membered heteroaryl. In certain embodiments, R¹ and R² are joined together with the intervening atoms to form unsubstituted aziridine, azetidine, pyrrolidine, piperidine, or piperazine.

In certain embodiments, X is —NR¹R² wherein R¹ and R² are joined together with the intervening atoms to form an optionally substituted heterocycle selected from the group consisting of

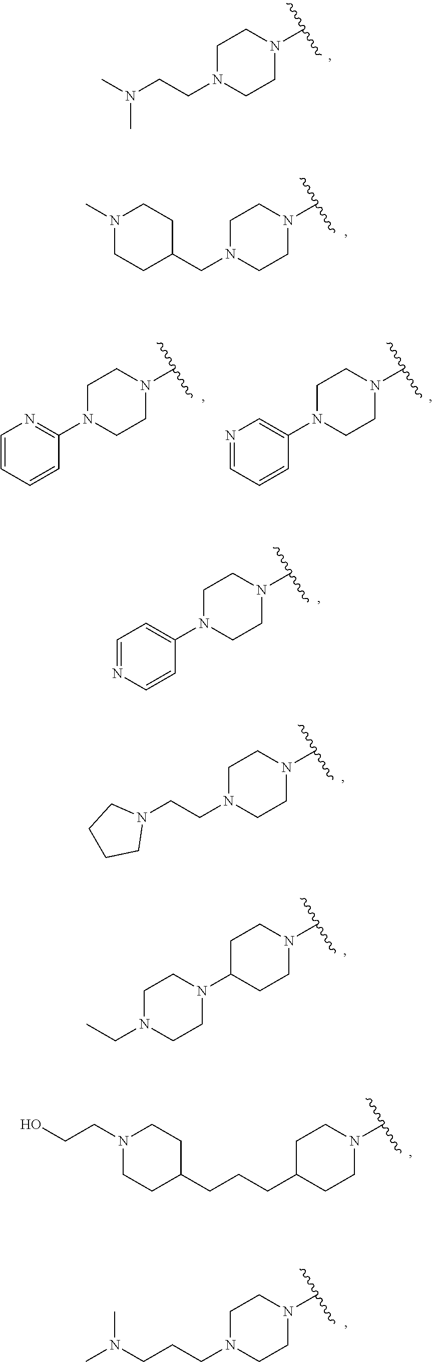

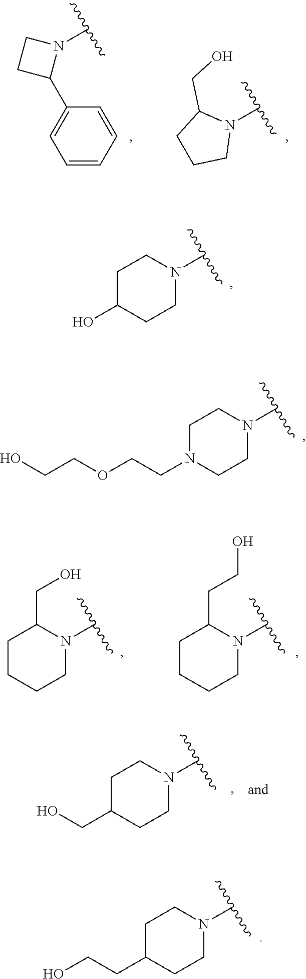

In certain embodiments, X is —NR¹R² wherein R¹ and R² are joined together with the intervening atoms to form an optionally substituted heterocycle selected from the group consisting of

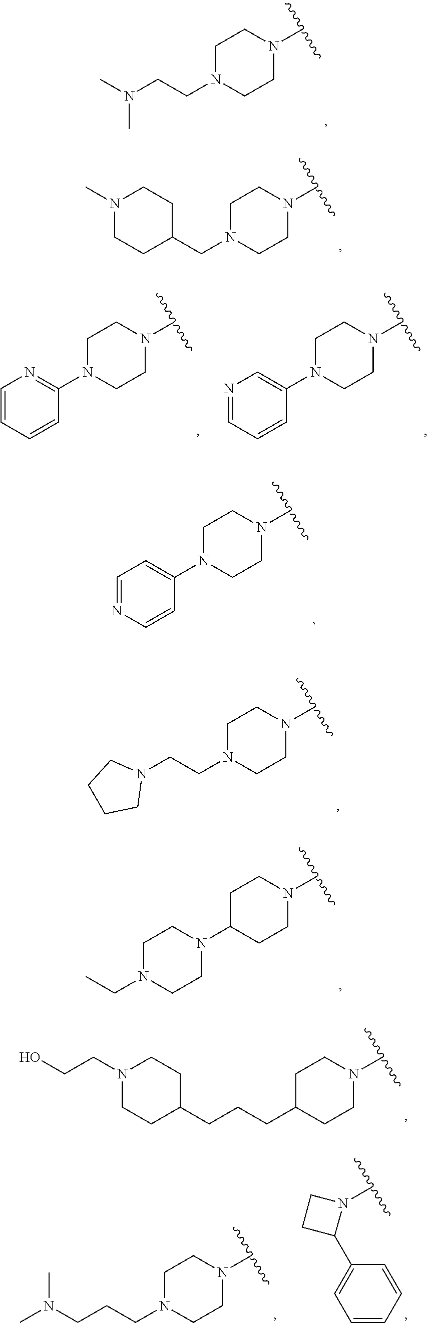

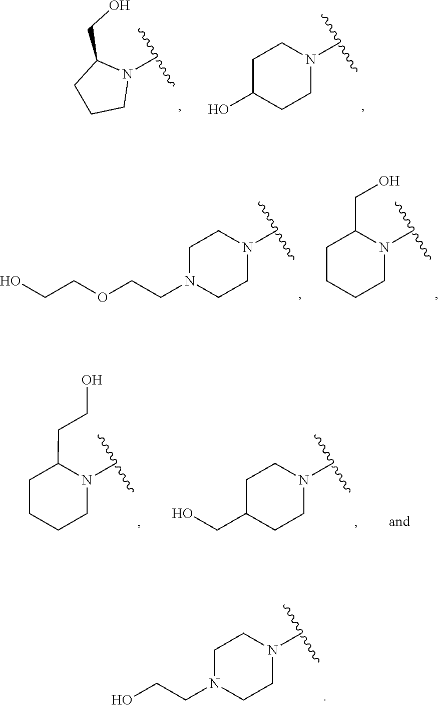

In some embodiments, R¹ and R² are joined together with the intervening atoms to form an optionally substituted heterocycle comprising one or more N atoms substituted with

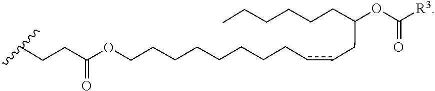

In certain embodiments, R¹ and R² are joined together with the intervening atoms to form an optionally substituted heterocycle comprising two N atoms substituted with

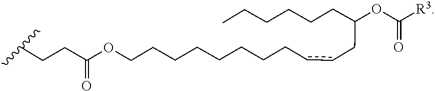

In some embodiments, R¹ and R² are joined together with the intervening atoms to form an optionally substituted heterocycle comprising three N atoms substituted with

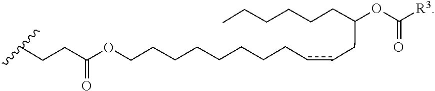

In some embodiments, R¹ and R² are joined together with the intervening atoms to form an optionally substituted heterocycle selected from the group consisting of:

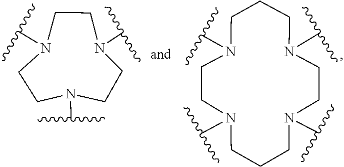 [SMILES: *N1CCN(*)CCN(*)CC1 ; *N1CCCN(*)CCN(*)CCCN(*)CC1]

wherein each N atom is substituted with

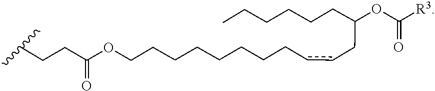

In certain embodiments,

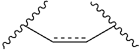

represents a single bond. In some embodiments,

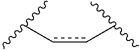

represents a double bond.

In certain embodiments, the compound of Formula (II) is:

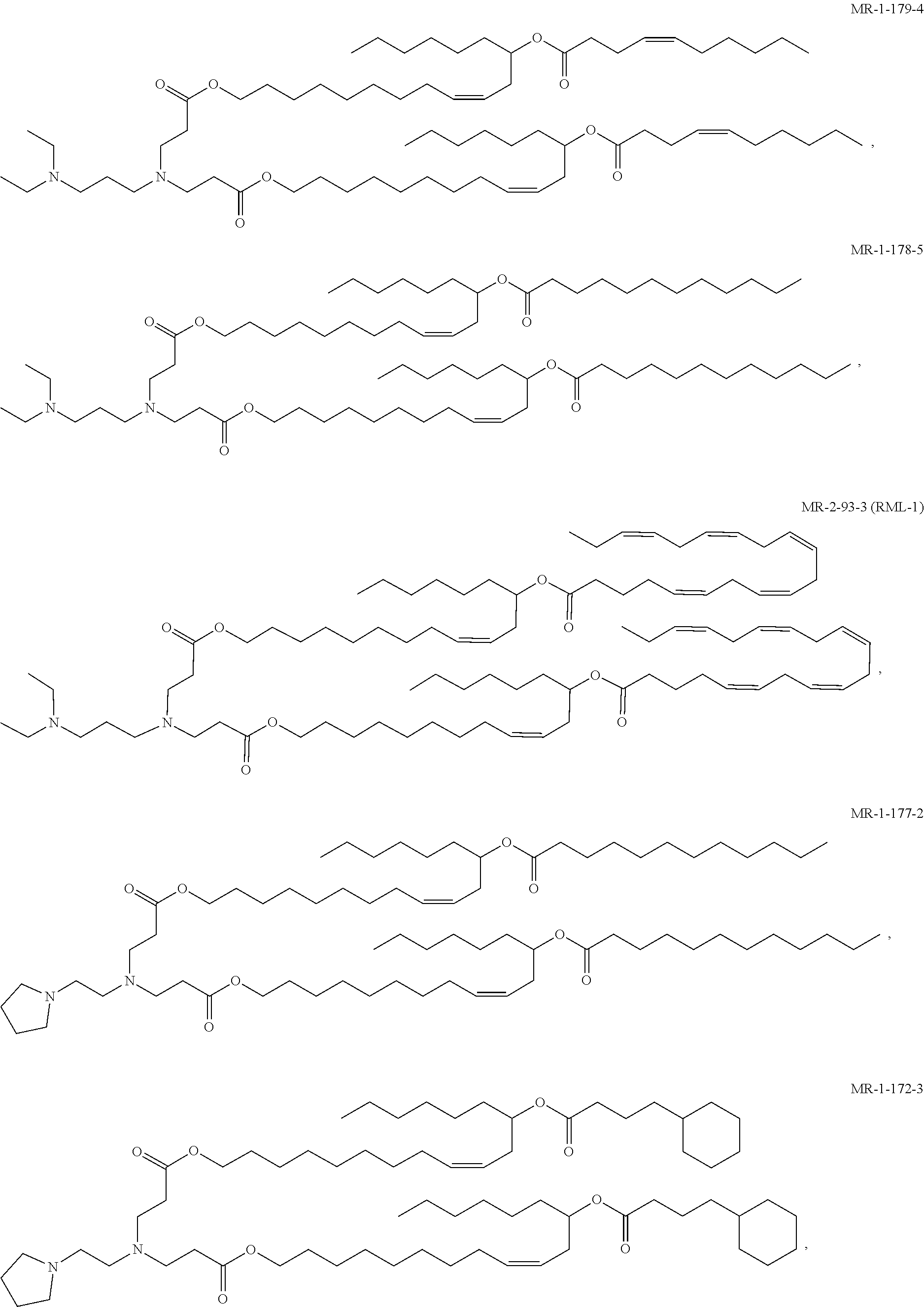

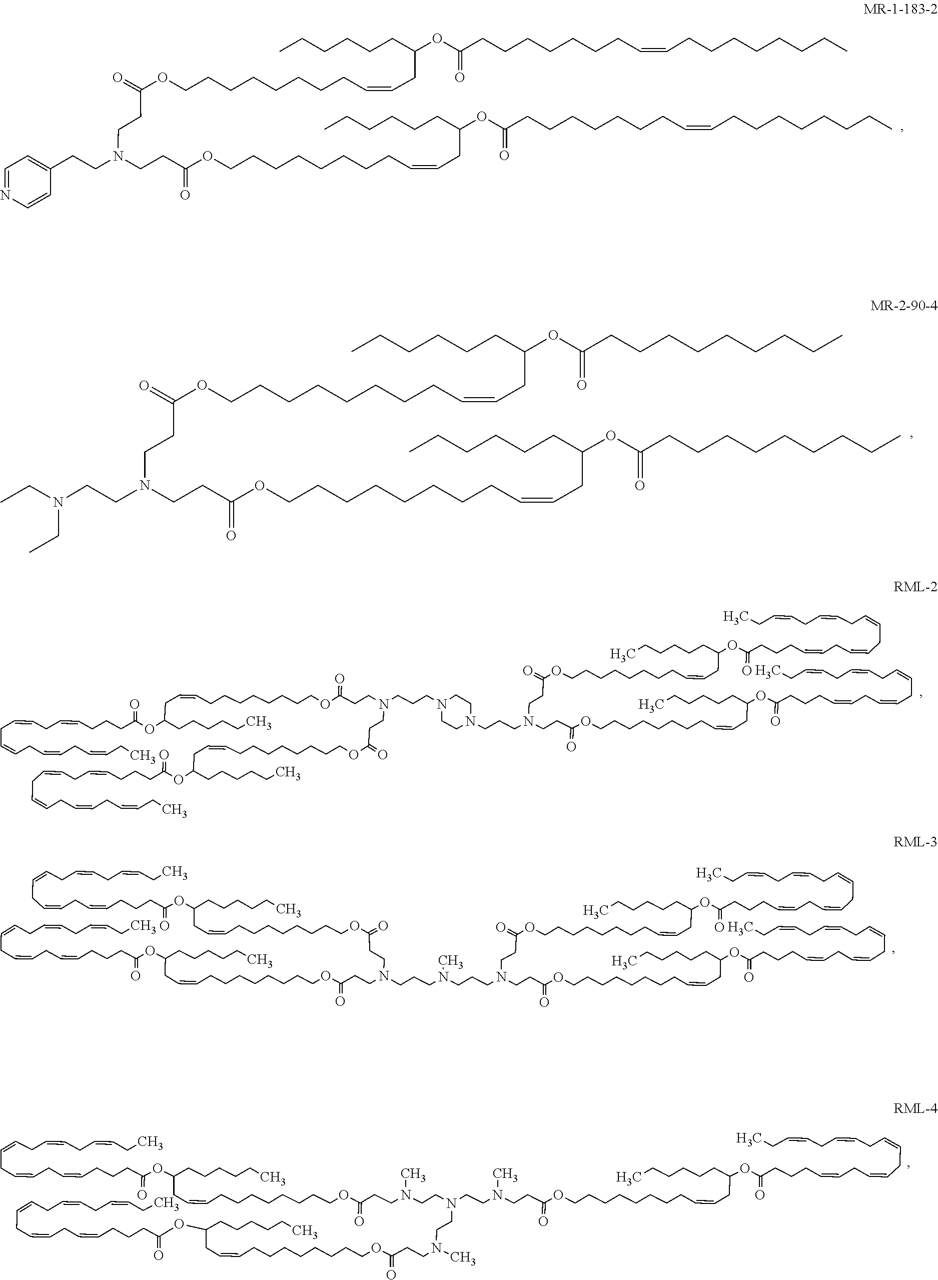

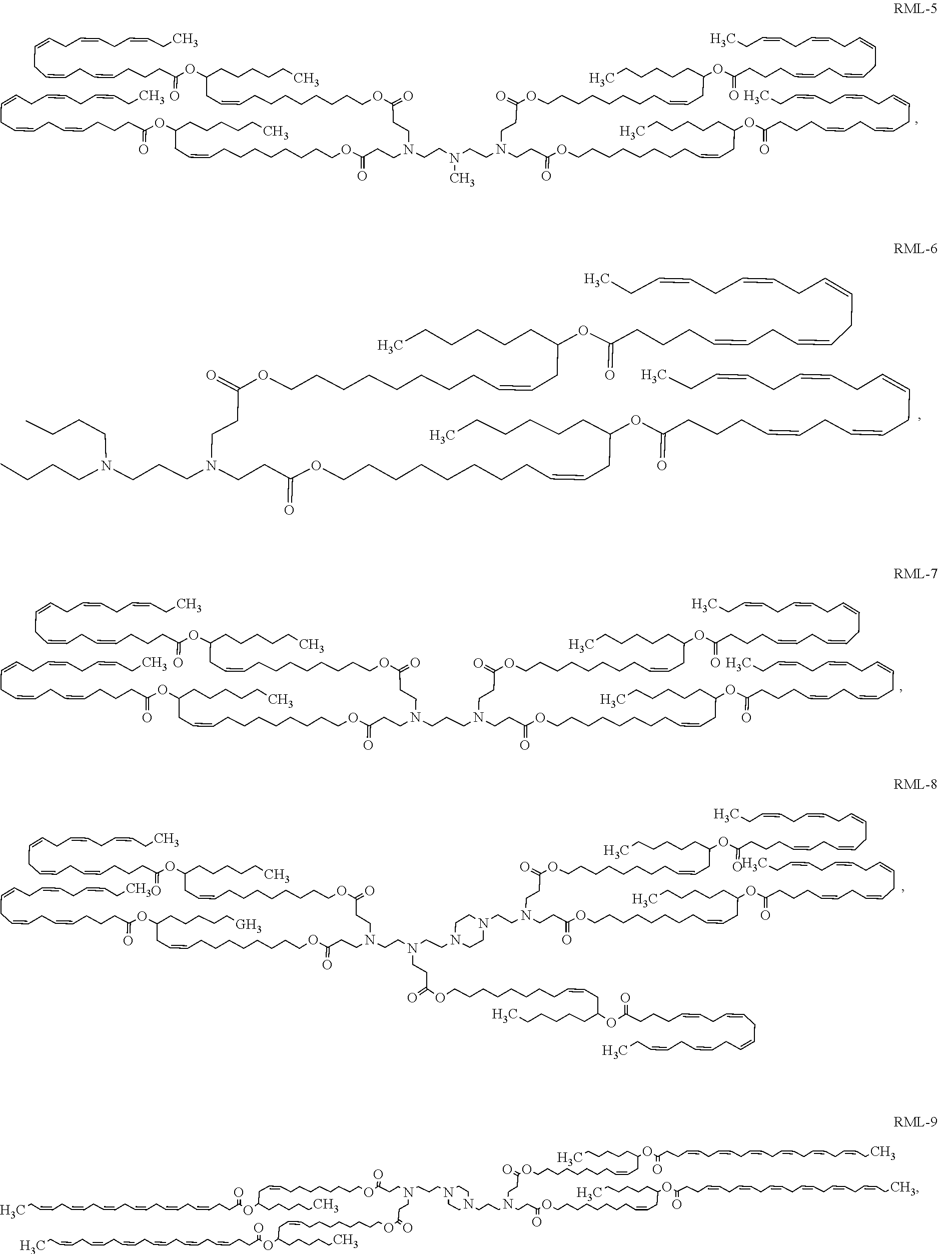

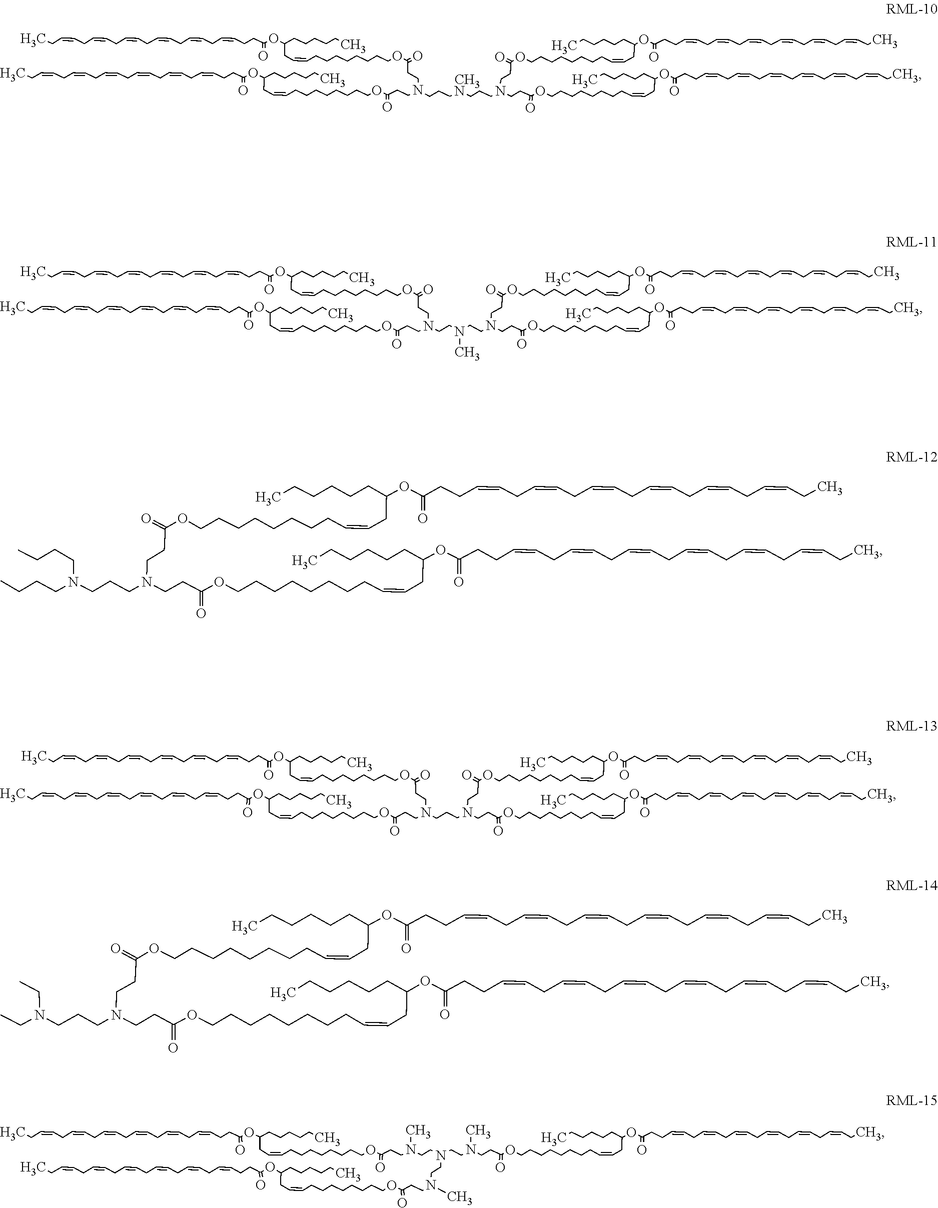

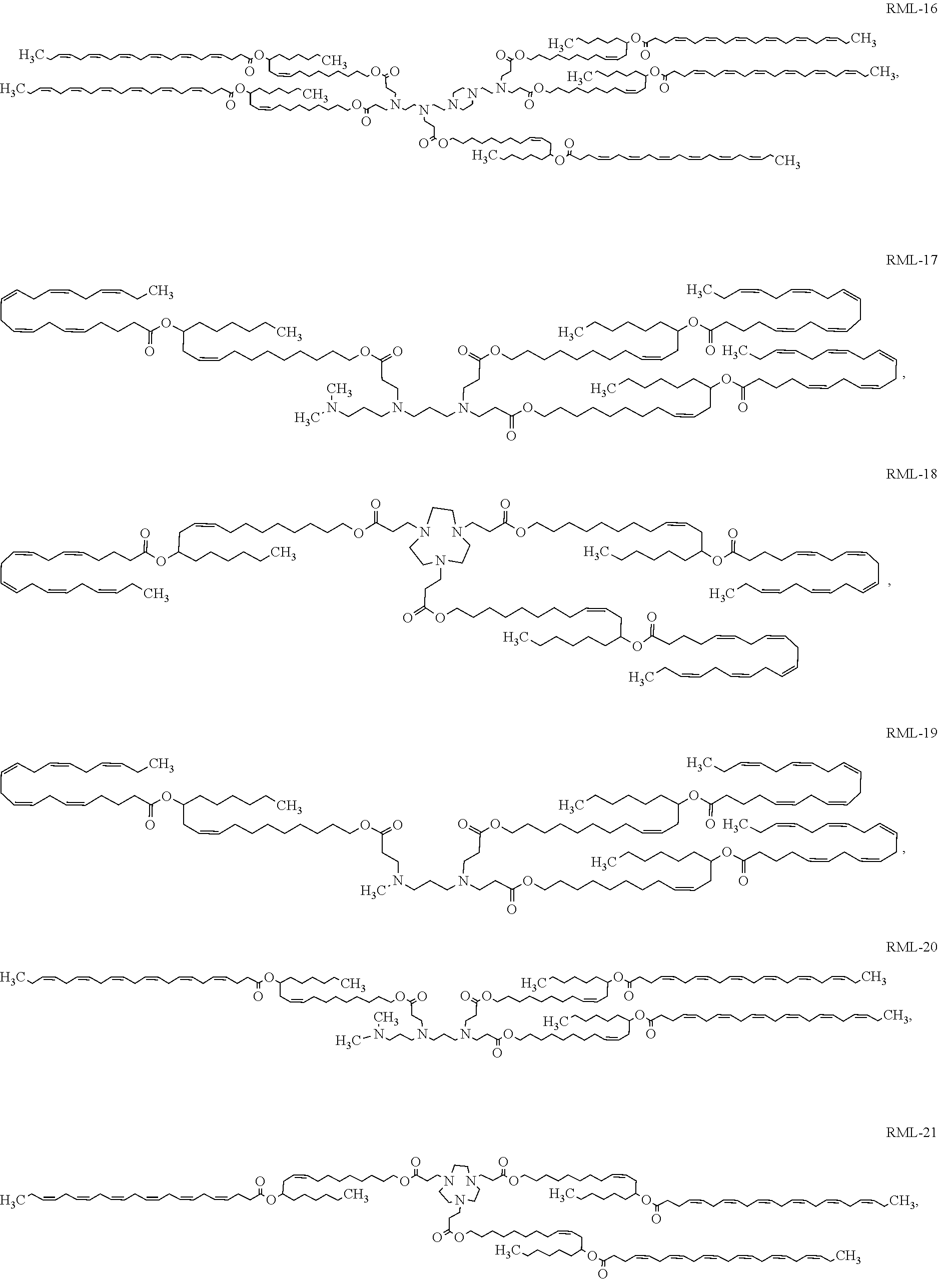

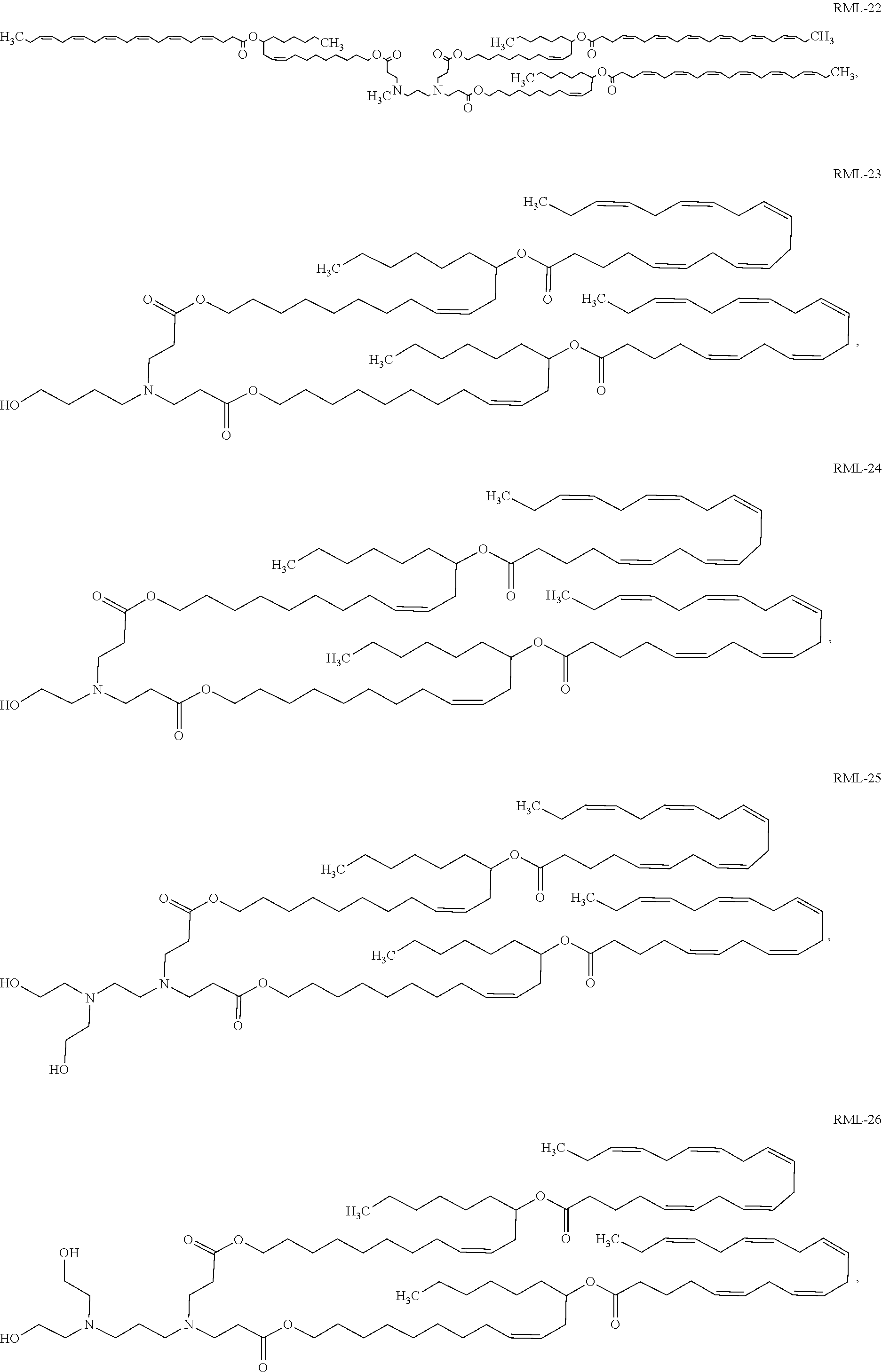

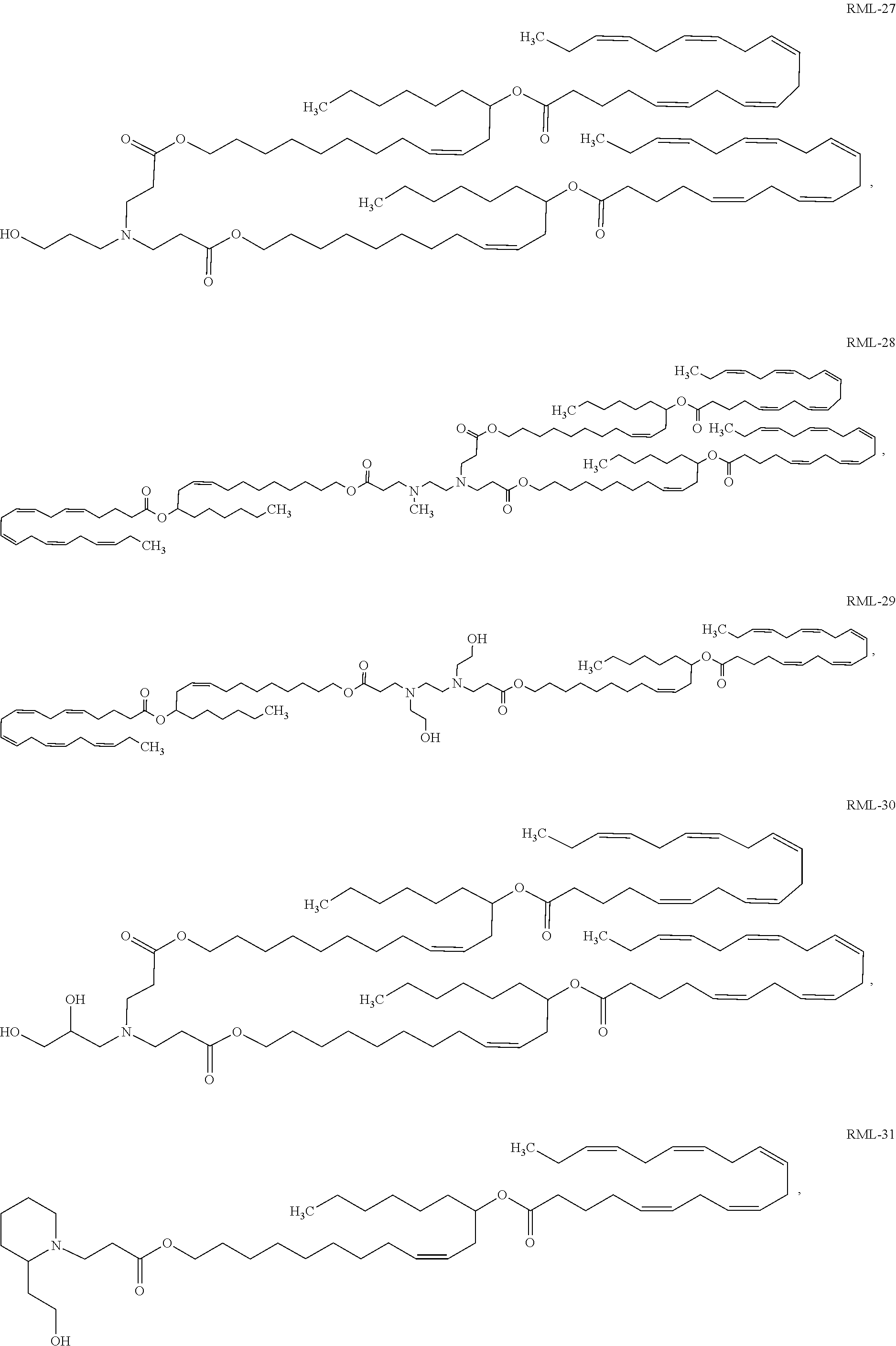

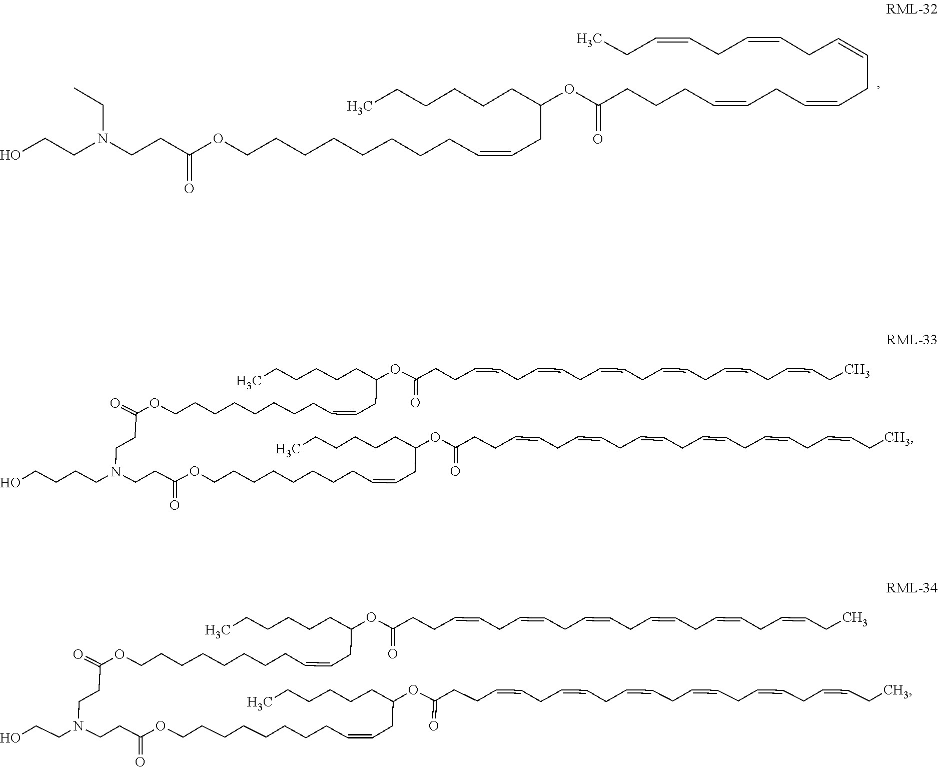 [SMILES: CC/C=C\C/C=C\C/C=C\C/C=C\C/C=C\CCCC(=O)OC(C/C=C\CCCCCCCCOC(=O)CCN(CC)CCO)CCCCCC ; CC/C=C\C/C=C\C/C=C\C/C=C\C/C=C\C/C=C\CCC(=O)OC(C/C=C\CCCCCCCCOC(=O)CCN(CCCCO)CCC(=O)OCCCCCCCC/C=C\CC(CCCCCC)OC(=O)CC/C=C\C/C=C\C/C=C\C/C=C\C/C=C\C/C=C\CC)CCCCCC ; CC/C=C\C/C=C\C/C=C\C/C=C\C/C=C\C/C=C\CCC(=O)OC(C/C=C\CCCCCCCCOC(=O)CCN(CCO)CCC(=O)OCCCCCCCC/C=C\CC(CCCCCC)OC(=O)CC/C=C\C/C=C\C/C=C\C/C=C\C/C=C\C/C=C\CC)CCCCCC]

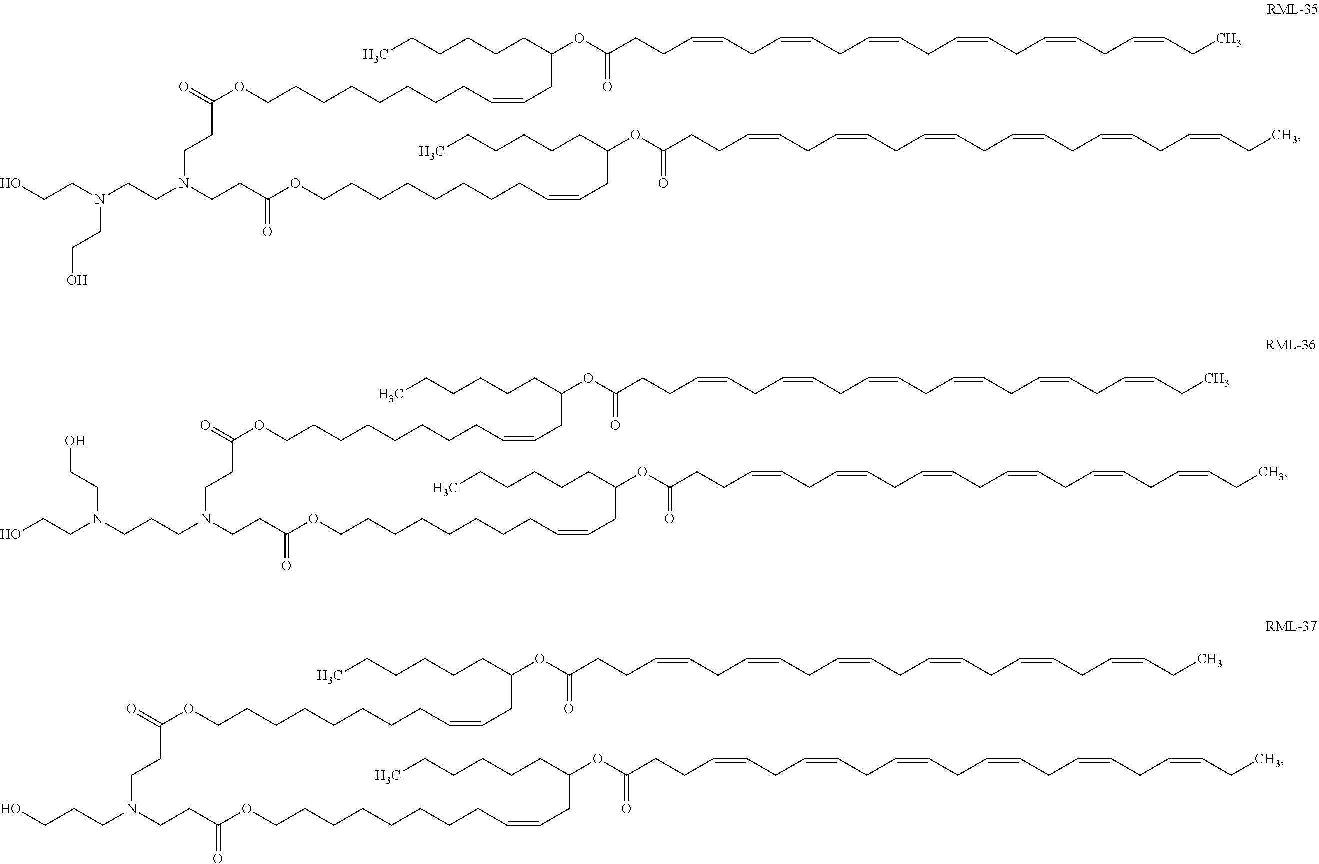 [SMILES: CC/C=C\C/C=C\C/C=C\C/C=C\C/C=C\C/C=C\CCC(=O)OC(C/C=C\CCCCCCCCOC(=O)CCN(CCC(=O)OCCCCCCCC/C=C\CC(CCCCCC)OC(=O)CC/C=C\C/C=C\C/C=C\C/C=C\C/C=C\C/C=C\CC)CCN(CCO)CCO)CCCCCC ; CC/C=C\C/C=C\C/C=C\C/C=C\C/C=C\C/C=C\CCC(=O)OC(C/C=C\CCCCCCCCOC(=O)CCN(CCCN(CCO)CCO)CCC(=O)OCCCCCCCC/C=C\CC(CCCCCC)OC(=O)CC/C=C\C/C=C\C/C=C\C/C=C\C/C=C\C/C=C\CC)CCCCCC ; CC/C=C\C/C=C\C/C=C\C/C=C\C/C=C\C/C=C\CCC(=O)OC(C/C=C\CCCCCCCCOC(=O)CCN(CCCO)CCC(=O)OCCCCCCCC/C=C\CC(CCCCCC)OC(=O)CC/C=C\C/C=C\C/C=C\C/C=C\C/C=C\C/C=C\CC)CCCCCC]

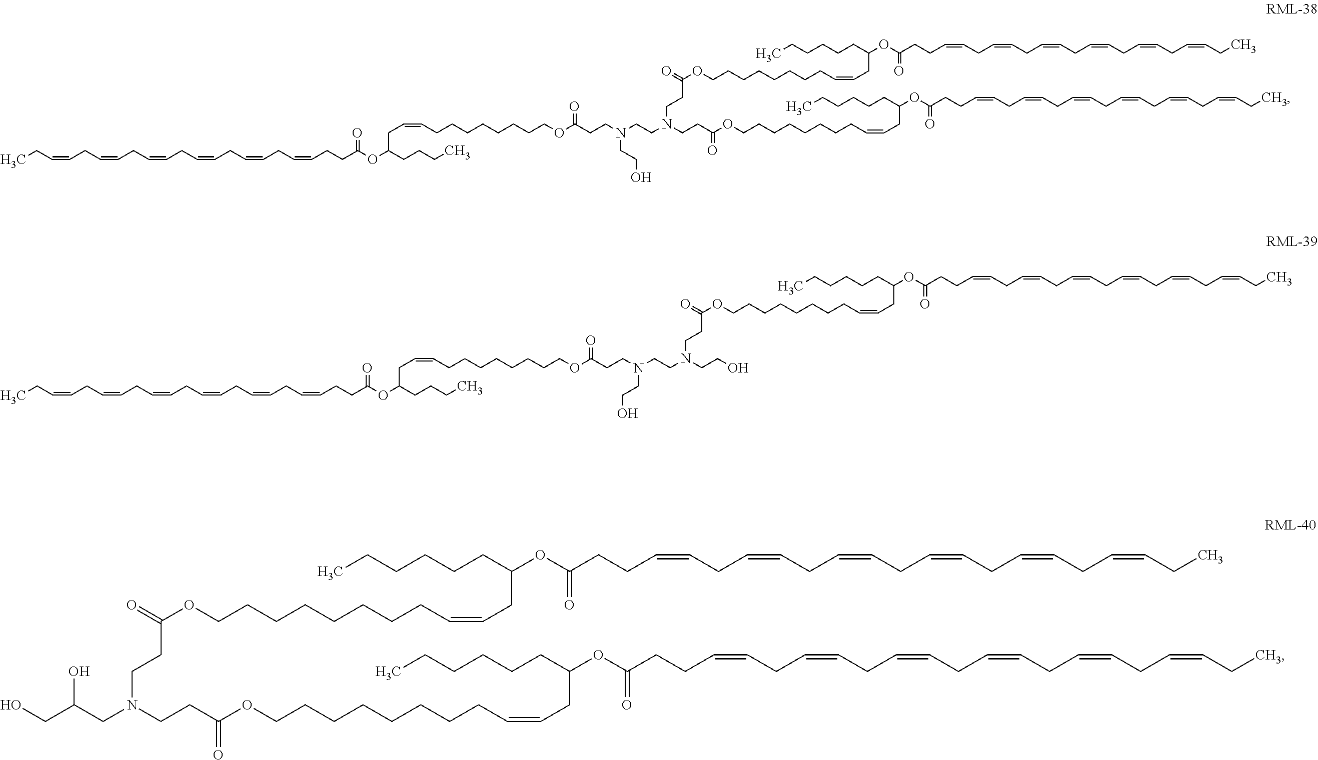

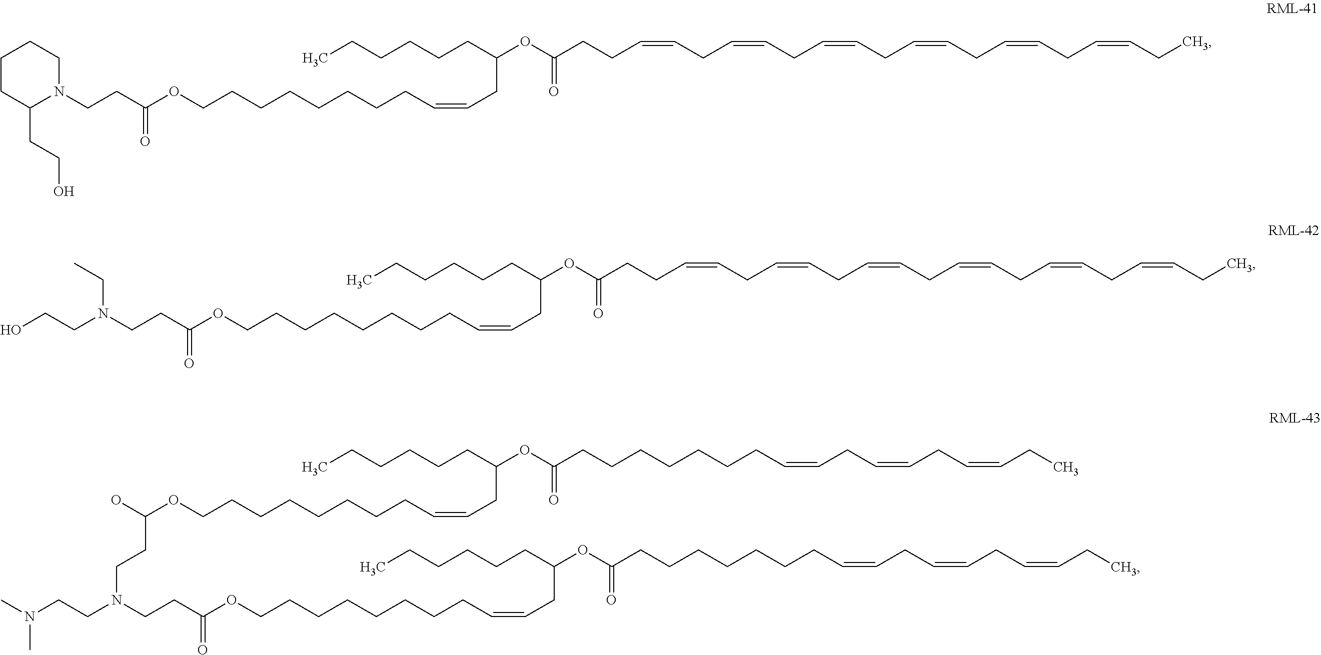 [SMILES: CC/C=C\C/C=C\C/C=C\C/C=C\C/C=C\C/C=C\CCC(=O)OC(C/C=C\CCCCCCCCOC(=O)CCN1CCCCC1CCO)CCCCCC ; CC/C=C\C/C=C\C/C=C\C/C=C\C/C=C\C/C=C\CCC(=O)OC(C/C=C\CCCCCCCCOC(=O)CCN(CC)CCO)CCCCCC ; CC/C=C\C/C=C\C/C=C\CCCCCCCC(=O)OC(C/C=C\CCCCCCCCOC(=O)CCN(CCC(O)OCCCCCCCC/C=C\CC(CCCCCC)OC(=O)CCCCCCC/C=C\C/C=C\C/C=C\CC)CCN(C)C)CCCCCC]

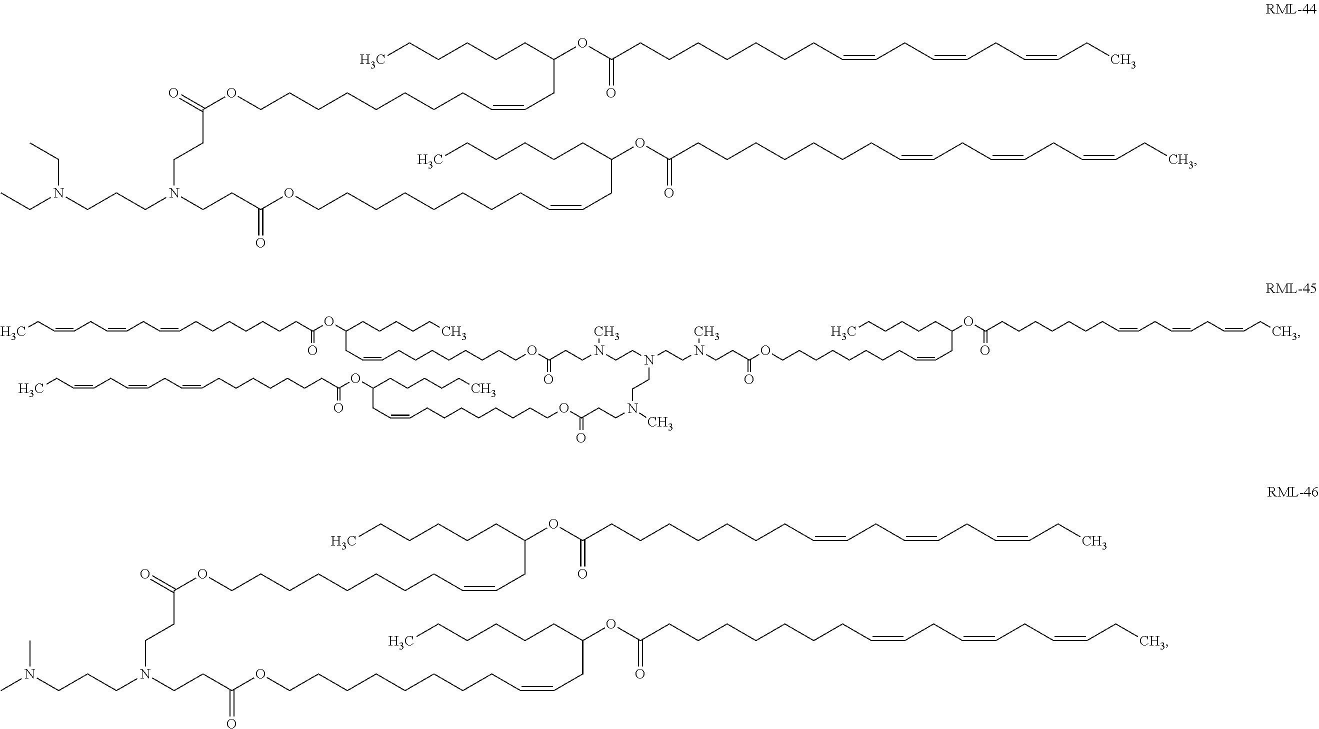 [SMILES: CC/C=C\C/C=C\C/C=C\CCCCCCCC(=O)OC(C/C=C\CCCCCCCCOC(=O)CCN(CCCN(CC)CC)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCC(=O)OC(C/C=C\CCCCCCCCOC(=O)CCN(C)CCN(CCN(C)CCC(=O)OCCCCCCCC/C=C\CC(CCCCCC)OC(=O)CCCCCCC/C=C\C/C=C\C/C=C\CC)CCN(C)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCC(=O)OC(C/C=C\CCCCCCCCOC(=O)CCN(CCCN(C)C)CCC(=O)OCCCCCCCC/C=C\CC(CCCCCC)OC(=O)CCCCCCC/C=C\C/C=C\C/C=C\CC)CCCCCC]

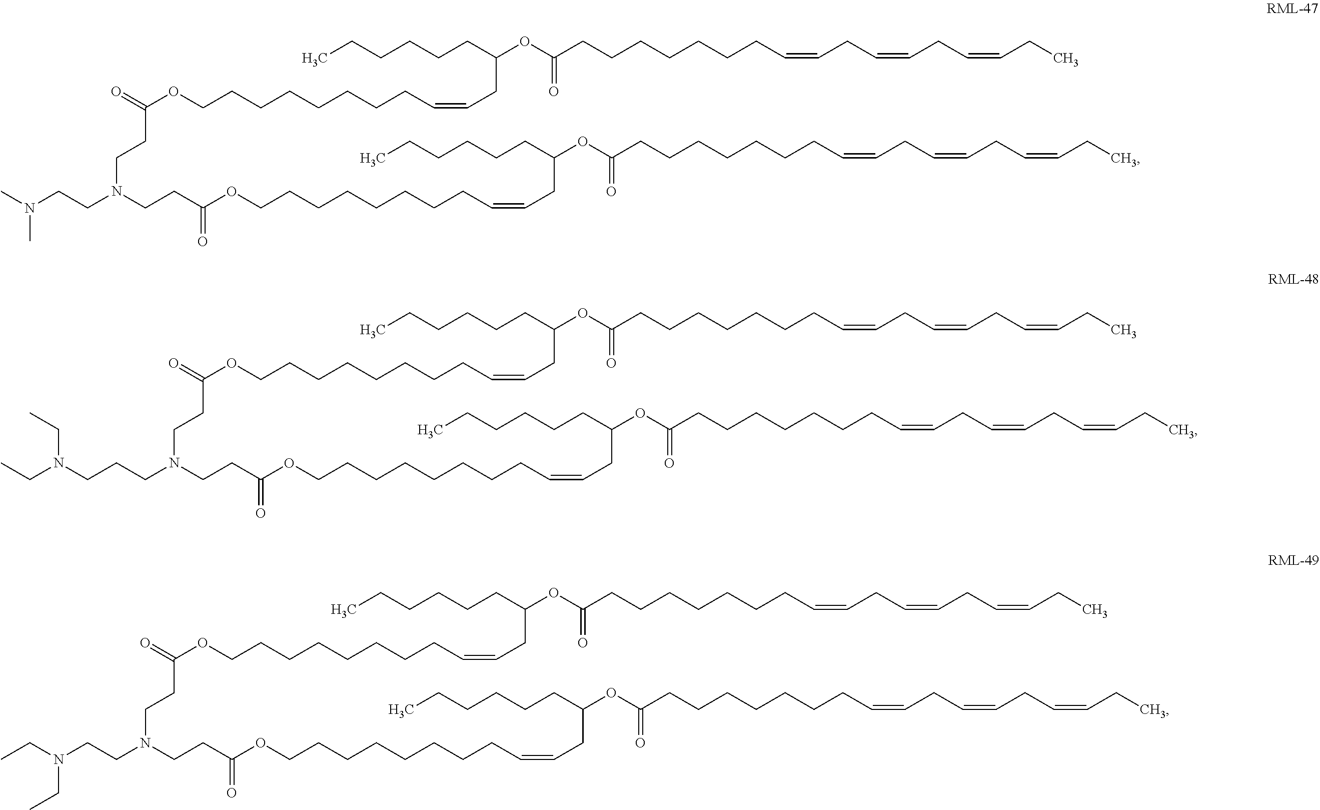 [SMILES: CC/C=C\C/C=C\C/C=C\CCCCCCCC(=O)OC(C/C=C\CCCCCCCCOC(=O)CCN(CCC(=O)OCCCCCCCC/C=C\CC(CCCCCC)OC(=O)CCCCCCC/C=C\C/C=C\C/C=C\CC)CCN(C)C)CCCCCC ; CC/C=C\C/C=C\C/C=C\CCCCCCCC(=O)OC(C/C=C\CCCCCCCCOC(=O)CCN(CCCN(CC)CC)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCC(=O)OC(C/C=C\CCCCCCCCOC(=O)CCN(CCC(=O)OCCCCCCCC/C=C\CC(CCCCCC)OC(=O)CCCCCCC/C=C\C/C=C\C/C=C\CC)CCN(CC)CC)CCCCCC]

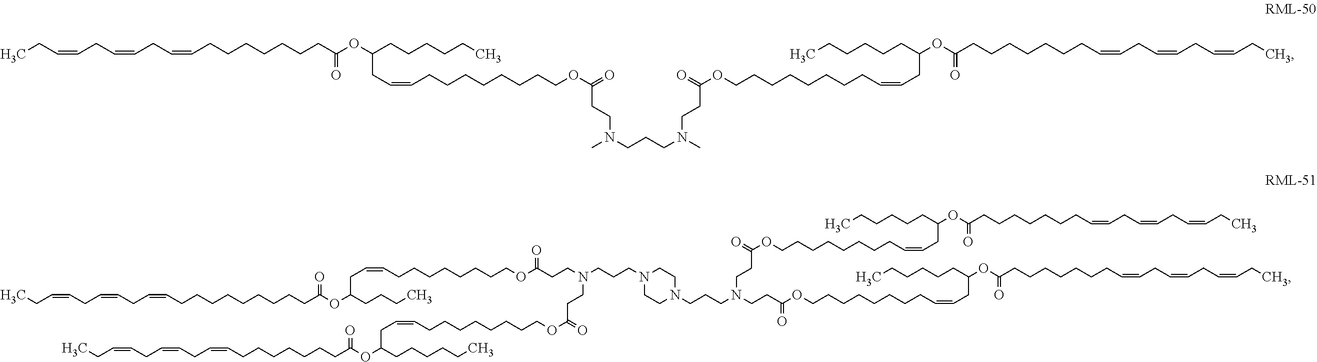 [SMILES: CC/C=C\C/C=C\C/C=C\CCCCCCCC(=O)OC(C/C=C\CCCCCCCCOC(=O)CCN(C)CCCN(C)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCCCC(=O)OC(C/C=C\CCCCCCCCOC(=O)CCN(CCCN1CCN(CCCN(CCC(=O)OCCCCCCCC/C=C\CC(CCCCCC)OC(=O)CCCCCCC/C=C\C/C=C\C/C=C\CC)CCC(=O)OCCCCCCCC/C=C\CC(CCCCCC)OC(=O)CCCCCCC/C=C\C/C=C\C/C=C\CC)CC1)CCC(=O)OCCCCCCCC/C=C\CC(CCCCCC)OC(=O)CCCCCCC/C=C\C/C=C\C/C=C\CC)CCCC]

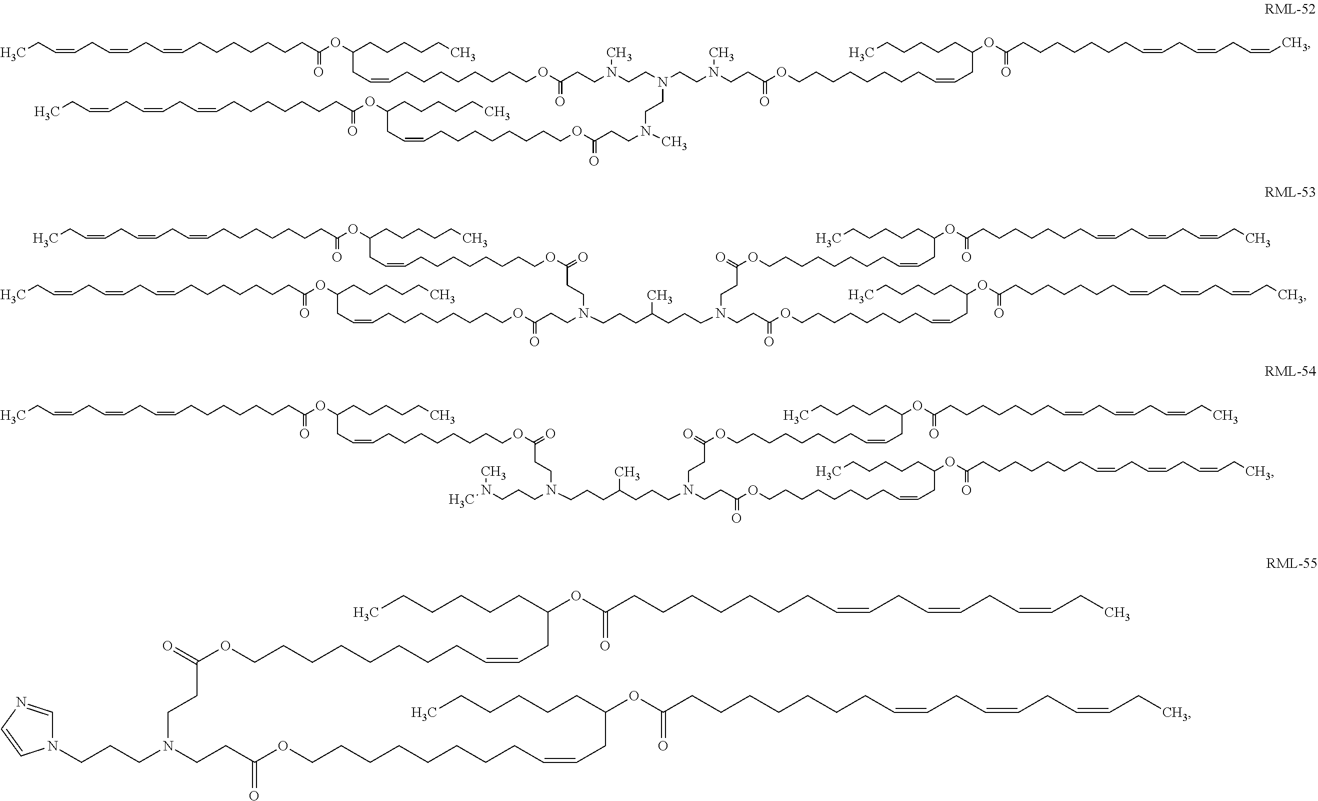

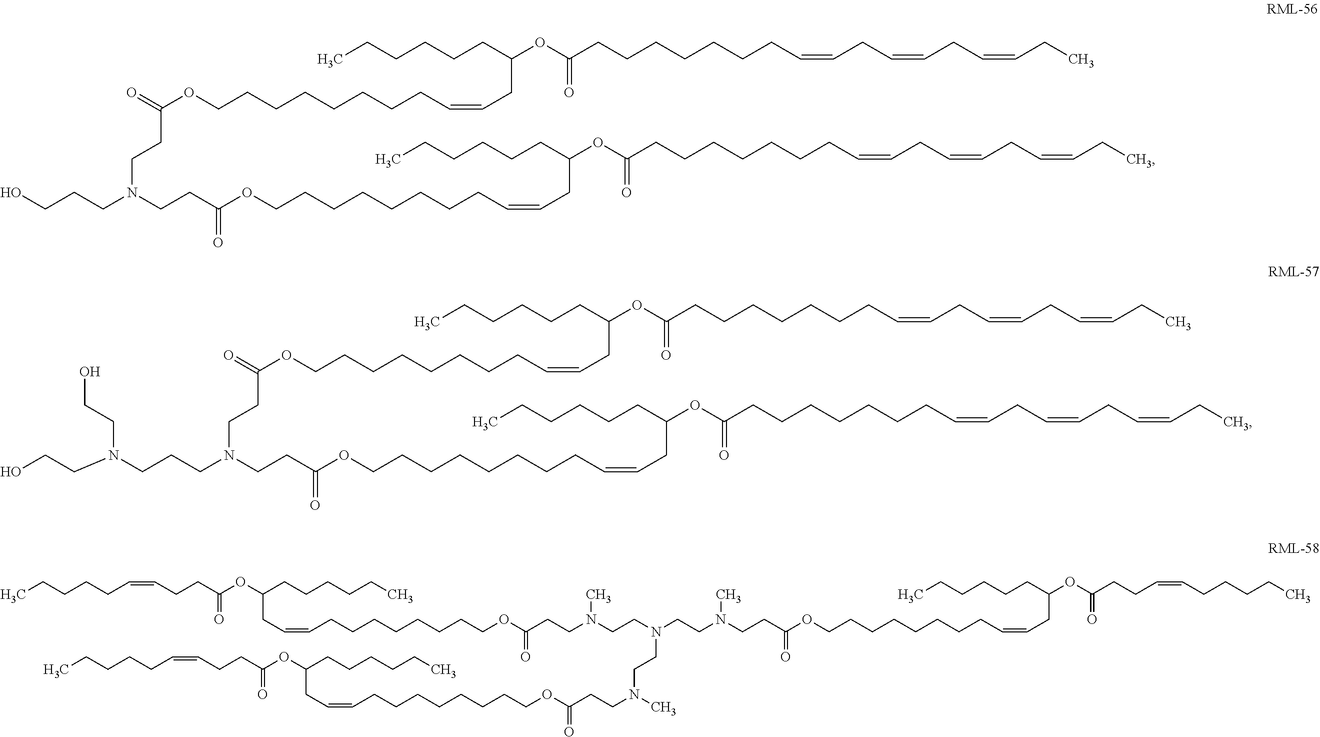 [SMILES: CC/C=C\C/C=C\C/C=C\CCCCCCCC(=O)OC(C/C=C\CCCCCCCCOC(=O)CCN(CCCO)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCC(=O)OC(C/C=C\CCCCCCCCOC(=O)CCN(CCCN(CCO)CCO)CCC(=O)OCCCCCCCC/C=C\CC(CCCCCC)OC(=O)CCCCCCC/C=C\C/C=C\C/C=C\CC)CCCCCC ; CCCCC/C=C\CCC(=O)OC(C/C=C\CCCCCCCCOC(=O)CCN(C)CCN(CCN(C)CCC(=O)OCCCCCCCC/C=C\CC(CCCCCC)OC(=O)CC/C=C\CCCCC)CCN(C)CCC(=O)OCCCCCCCC/C=C\CC(CCCCCC)OC(=O)CC/C=C\CCCCC)CCCCCC]

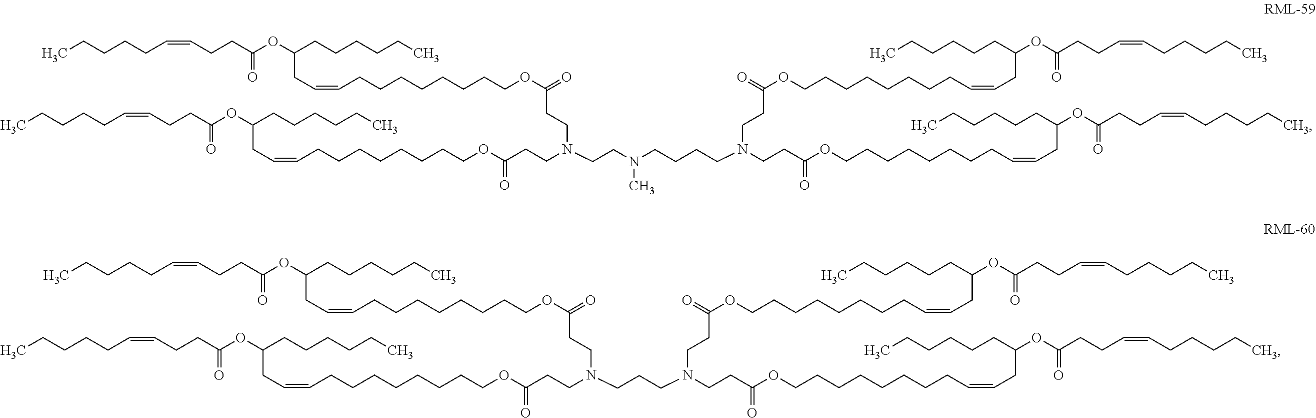 [SMILES: CCCCC/C=C\CCC(=O)OC(C/C=C\CCCCCCCCOC(=O)CCN(CCCCN(C)CCN(CCC(=O)OCCCCCCCC/C=C\CC(CCCCCC)OC(=O)CC/C=C\CCCCC)CCC(=O)OCCCCCCCC/C=C\CC(CCCCCC)OC(=O)CC/C=C\CCCCC)CCC(=O)OCCCCCCCC/C=C\CC(CCCCCC)OC(=O)CC/C=C\CCCCC)CCCCCC ; CCCCC/C=C\CCC(=O)OC(C/C=C\CCCCCCCCOC(=O)CCN(CCCN(CCC(=O)OCCCCCCCC/C=C\CC(CCCCCC)OC(=O)CC/C=C\CCCCC)CCC(=O)OCCCCCCCC/C=C\CC(CCCCCC)OC(=O)CC/C=C\CCCCC)CCC(=O)OCCCCCCCC/C=C\CC(CCCCCC)OC(=O)CC/C=C\CCCCC)CCCCCC]

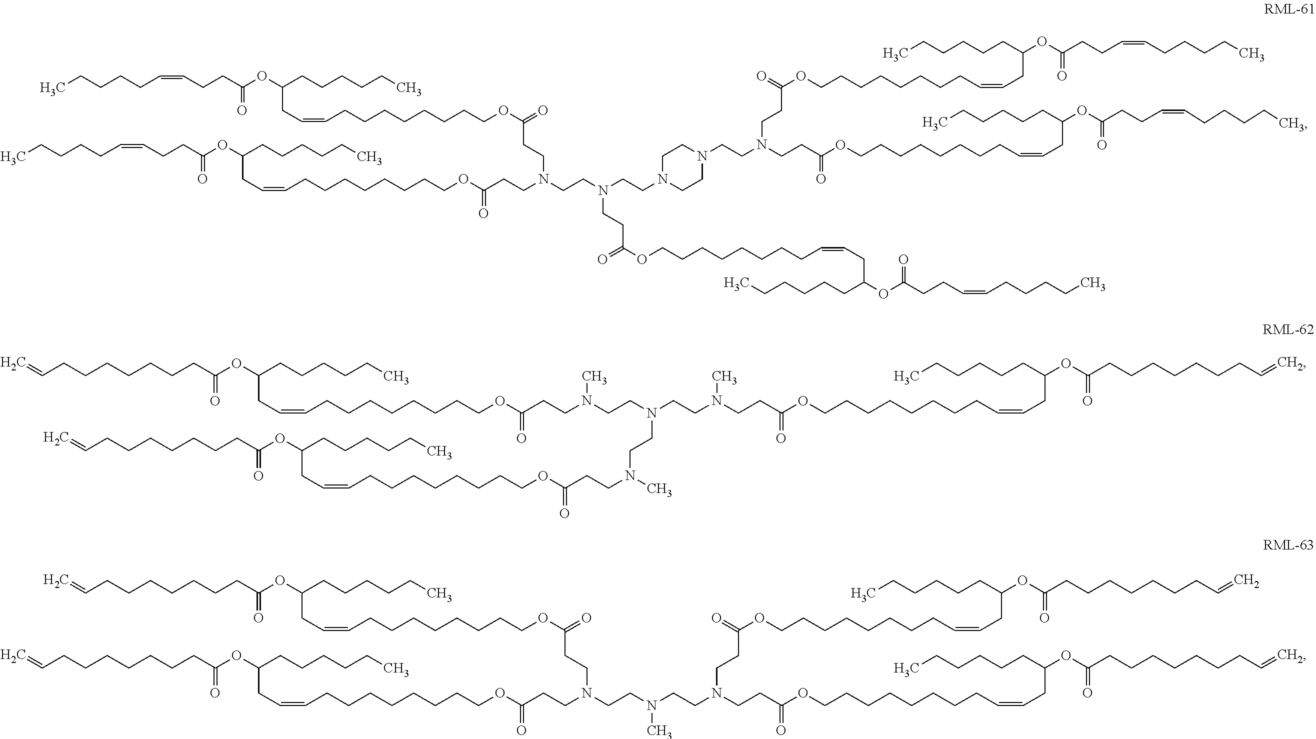

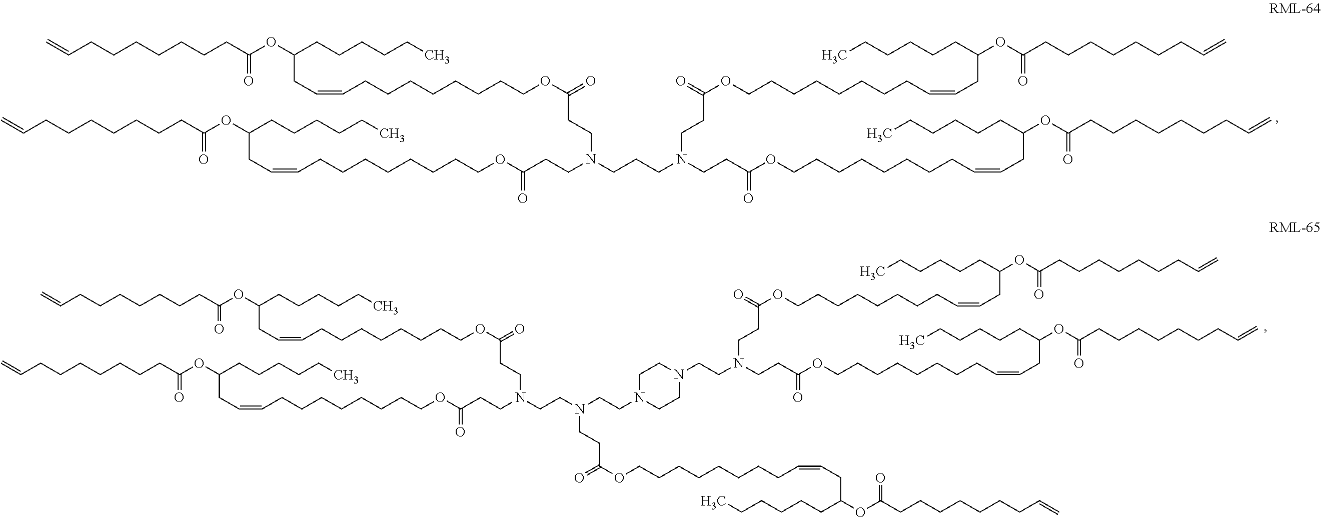

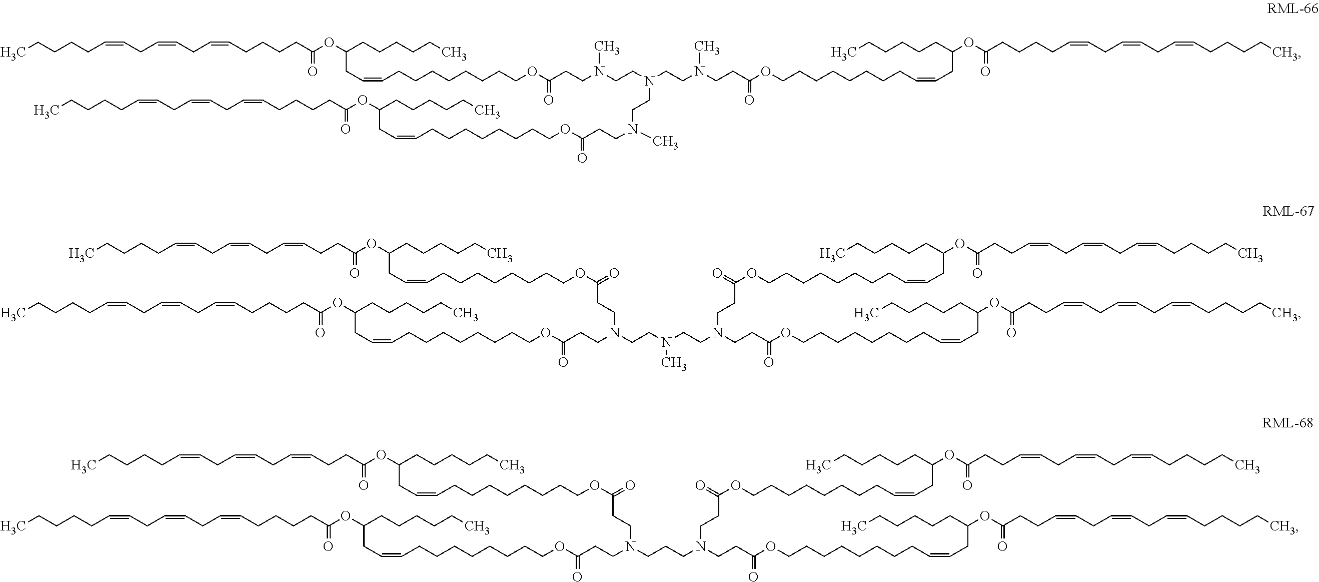

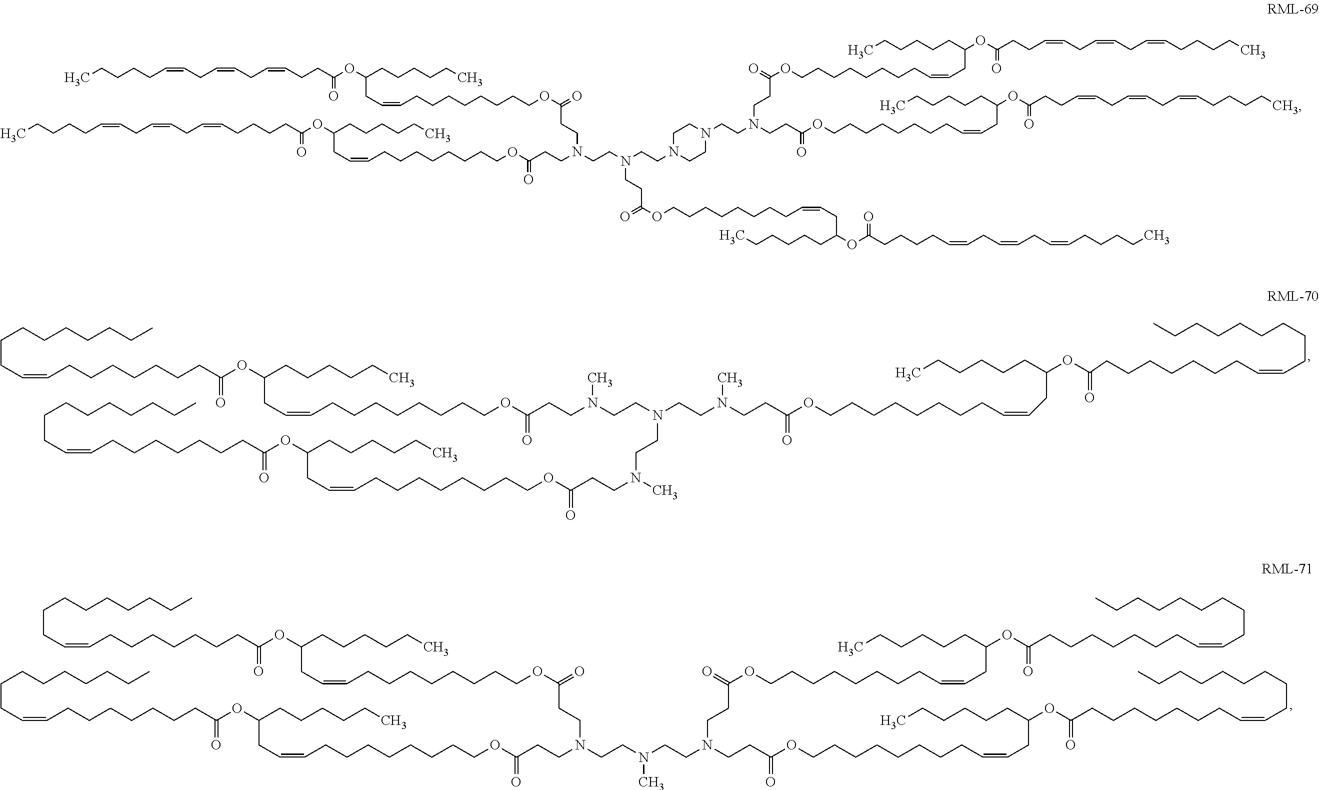

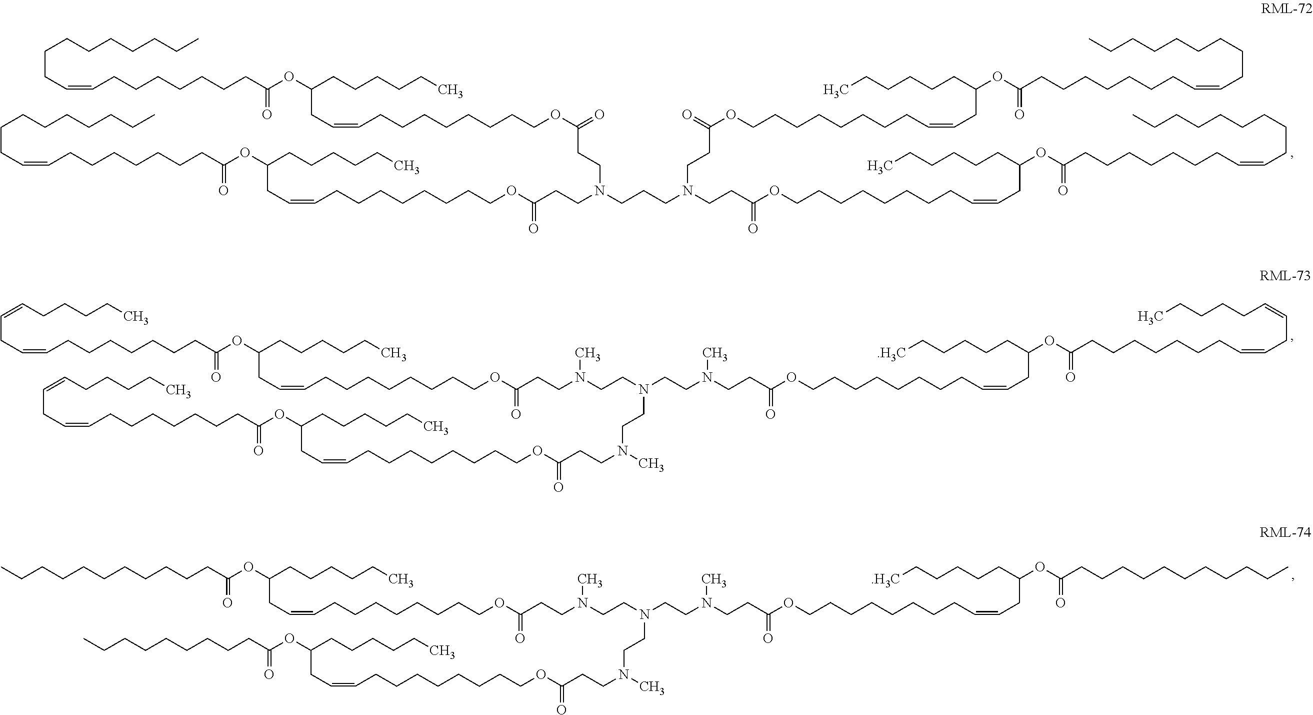

or a pharmaceutically acceptable salt, solvate, tautomer, stereoisomer, or isotopically labeled derivative thereof.

In certain embodiments, the compound of Formula (II) is:

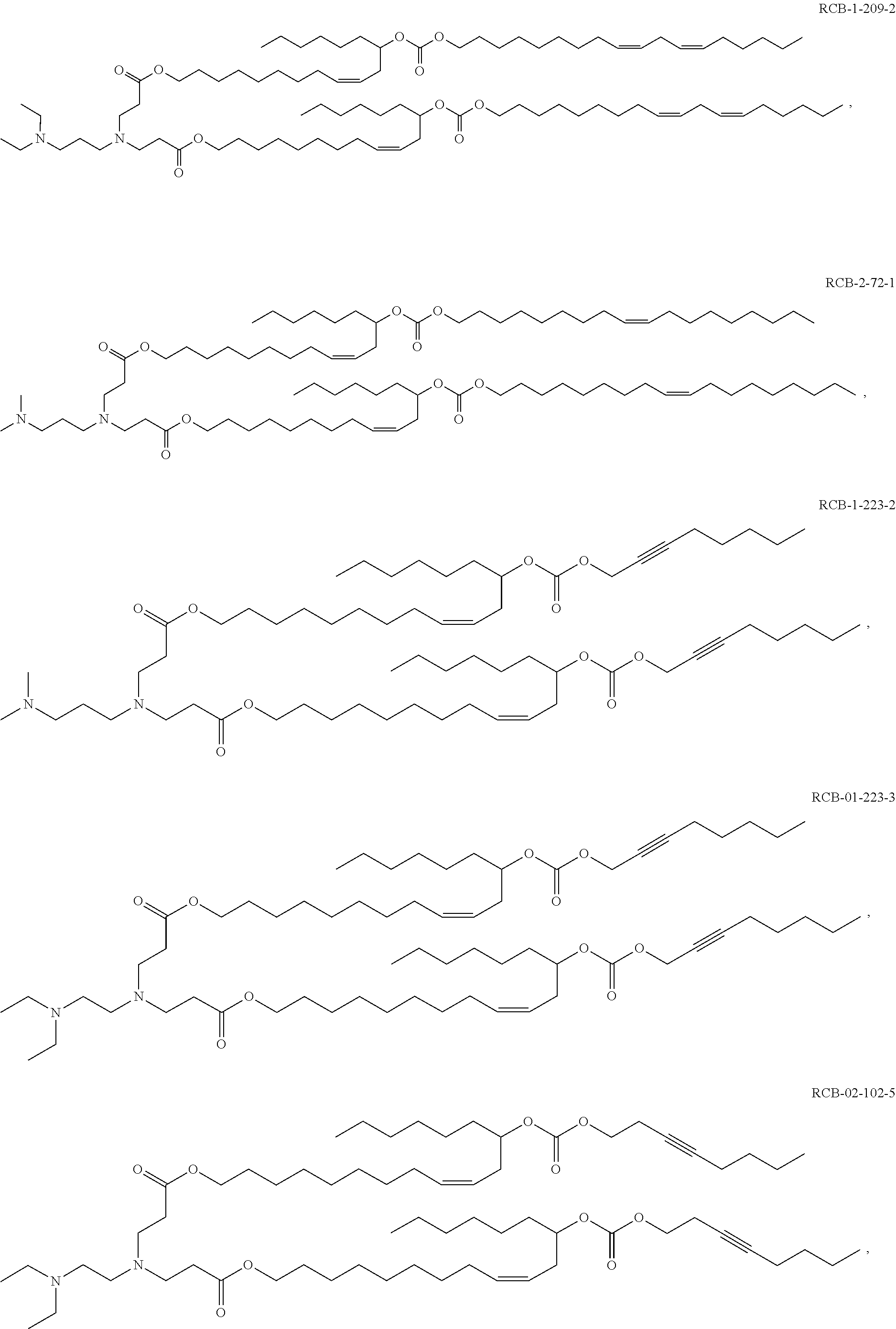

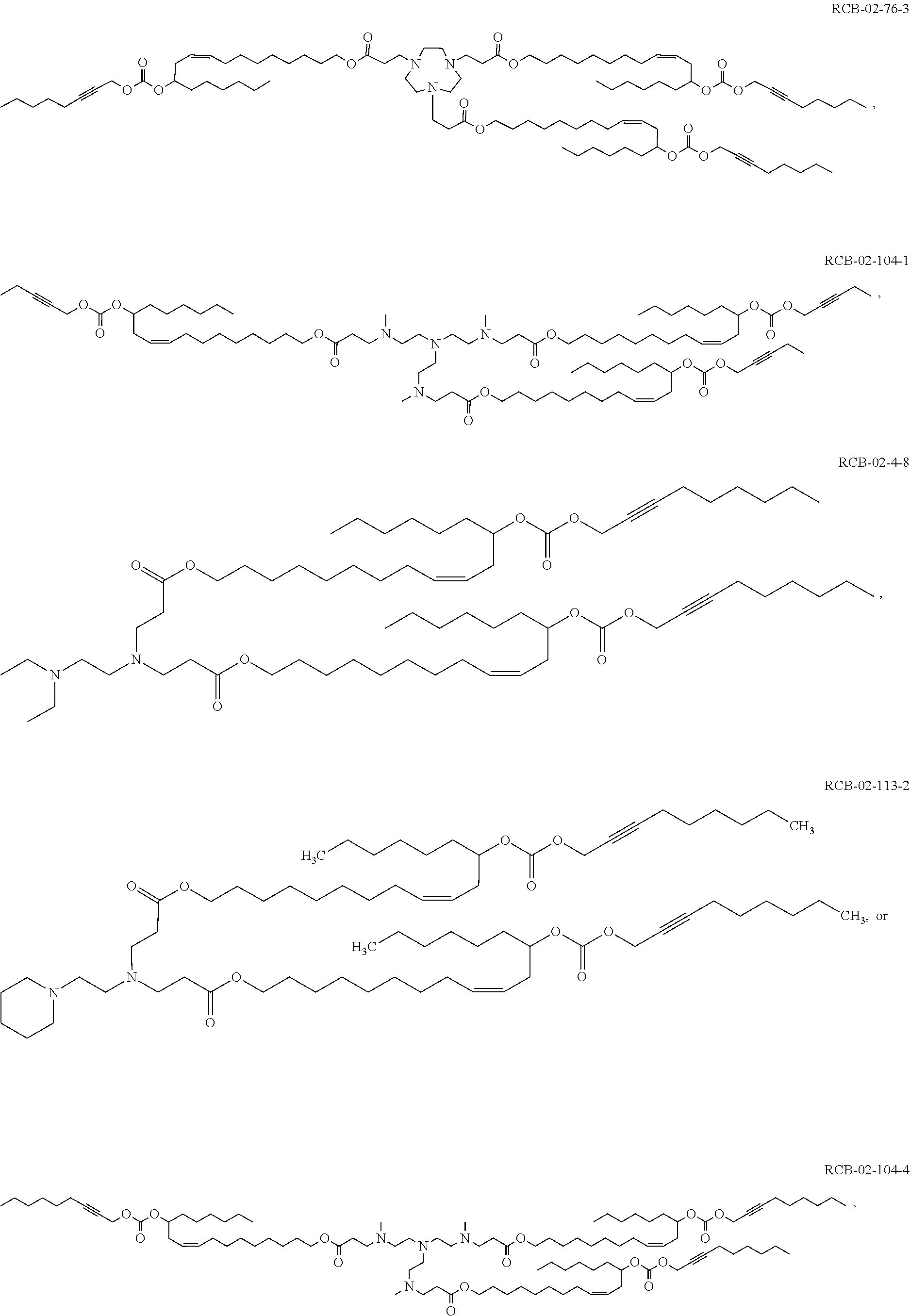

or a pharmaceutically acceptable salt, solvate, tautomer, stereoisomer, or isotopically labeled derivative thereof.

In another aspect, the disclosure provides a compound prepared by reacting a compound of Formula (III):

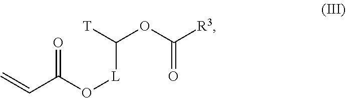

or a salt thereof, with a compound of formula:
or a salt thereof, wherein:
T is optionally substituted C₁-C₂₀ alkyl, optionally substituted C₂-C₂₀ alkenyl, or optionally substituted C₂-C₂₀ alkynyl;
L is optionally substituted C₁-C₂₀ alkylene, optionally substituted C₂-C₂₀ alkenylene, or optionally substituted C₂-C₂₀ alkynylene;
R³ is optionally substituted aliphatic or optionally substituted heteroaliphatic;
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group; and
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl.

In another aspect, the disclosure provides a compound prepared by reacting a compound of Formula (IV):

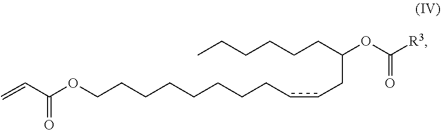

or a salt thereof, with a compound of formula:

H—X,

or a salt thereof, wherein:
R³ is optionally substituted aliphatic or optionally substituted heteroaliphatic;
X is —OR¹, —SR¹, or —NR¹R²;
R¹ is optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl; and

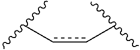

represents either a single or a double bond.

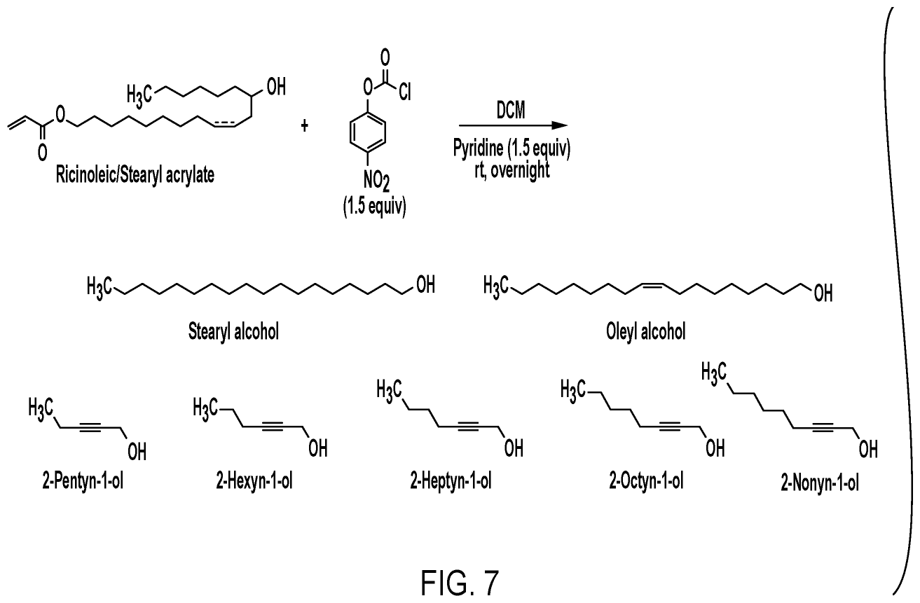

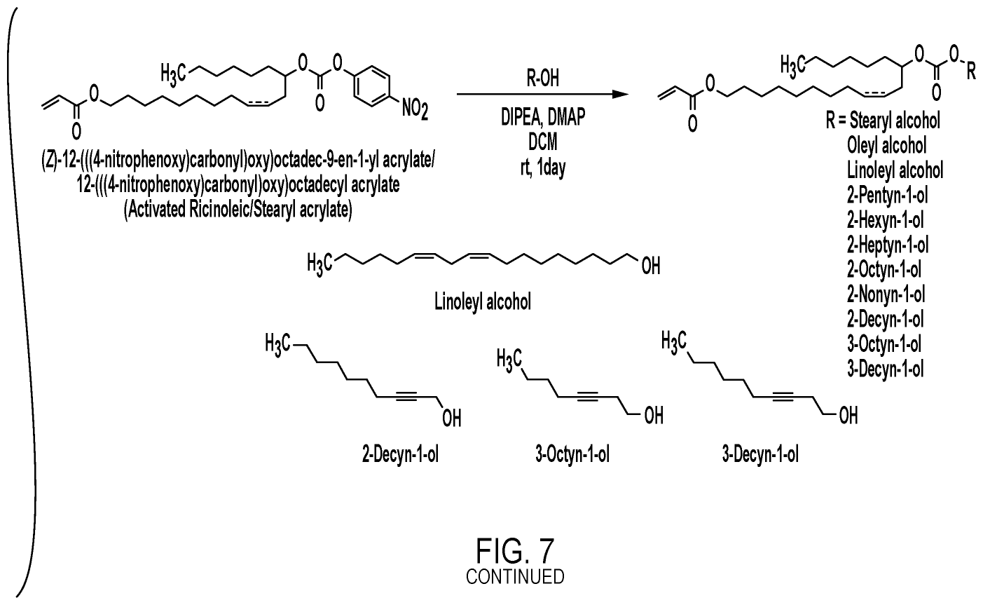

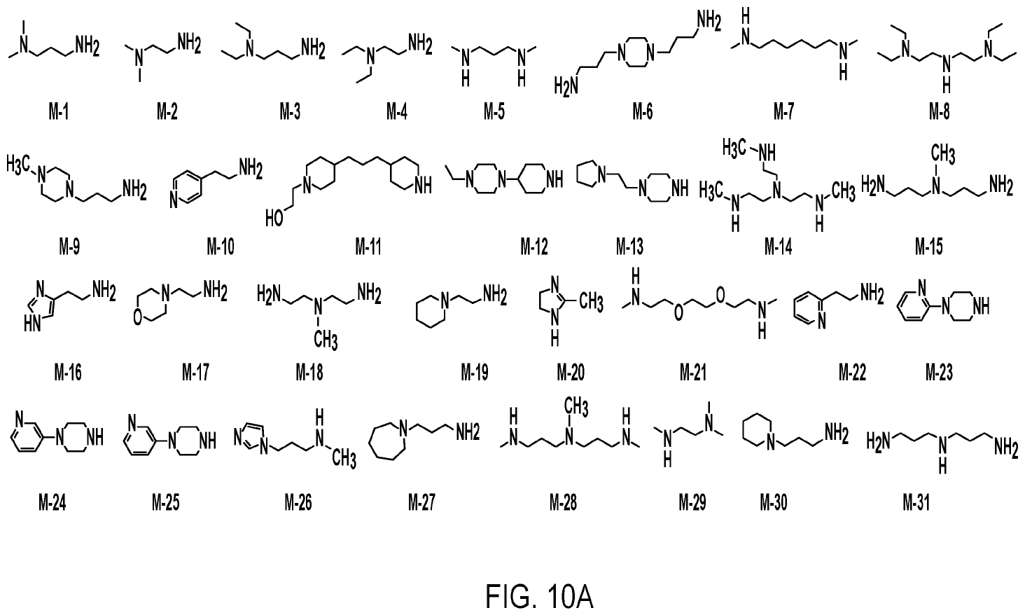

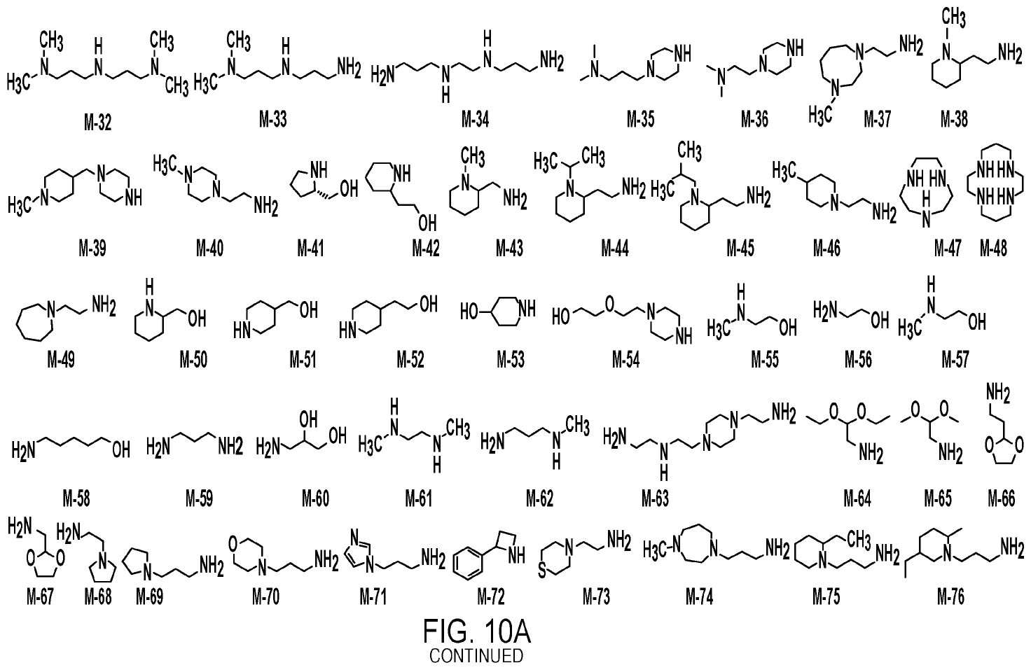

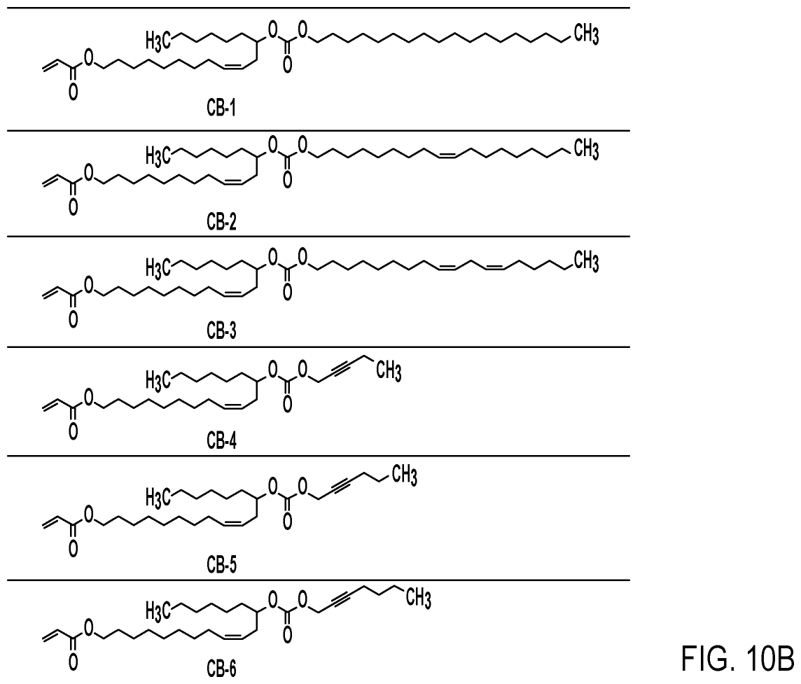

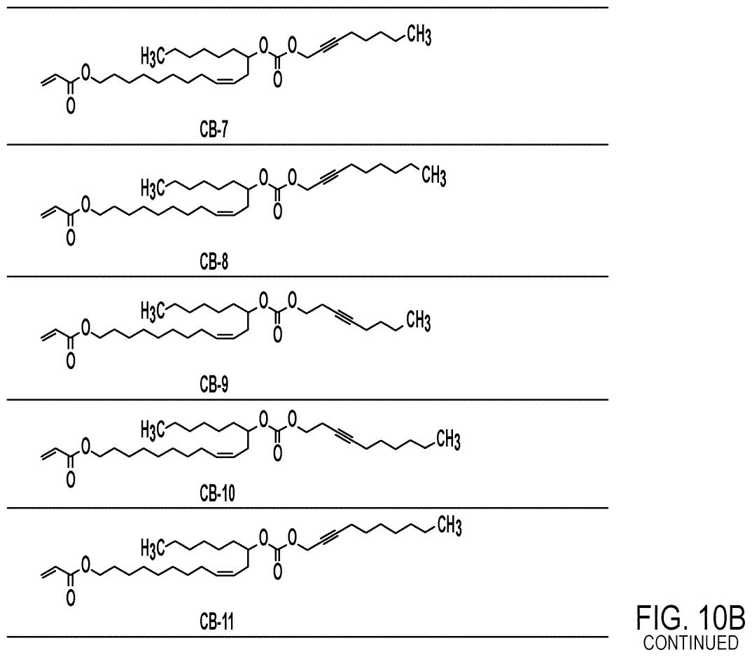

In some embodiments, the compound of Formula (IV) is selected from an acrylate provided in FIG. 7. In some embodiments, the compound of Formula (IV) is selected from an acrylate provided in FIG. 10B. In some embodiments, the compound of Formula (IV) is selected from an acrylate provided in FIG. 7 or FIG. 10B.

As defined herein, R³ is optionally substituted aliphatic or optionally substituted heteroaliphatic. In some embodiments, R³ is optionally substituted aliphatic, —OR^{3O}, or —N(R^{3N})₂, wherein each instance of R^{3O} and R^{3N} is independently optionally substituted aliphatic. In certain embodiments, R³ is C₁-C₂₅ optionally substituted aliphatic, —OR^{3O}, or —N(R^{3N})₂, wherein each instance of R^{3O} and R^{3N} is independently optionally substituted C₁-C₂₅ aliphatic. In some embodiments, R³ is optionally substituted aliphatic or —OR^{3O}, wherein R^{3O} is optionally substituted aliphatic. In certain embodiments, R³ is C₁-C₂₅ optionally substituted aliphatic or —OR^{3O}, wherein R^{3O} is optionally substituted C₁-C₂₅ aliphatic.

In some embodiments, R³ is C₁-C₂₅ optionally substituted aliphatic. In some embodiments, R³ is C₁-C₂₅ substituted aliphatic. In certain embodiments, R³ is C₁-C₂₅ unsubstituted aliphatic. In some embodiments, R³ is C₁-C₂₅ optionally substituted alkyl. In some embodiments, R³ is C₁-C₂₅ substituted alkyl. In certain embodiments, R³ is C₁-C₂₅ unsubstituted alkyl. In some embodiments, R³ is C₁-C₂₅ alkyl substituted with C₃-C₈ unsubstituted cycloalkyl. In some embodiments, R³ is optionally substituted C₁-C₁₅ alkyl. In some embodiments, R³ is substituted C₁-C₁₅ alkyl. In some embodiments, R³ is unsubstituted C₁-C₁₅ alkyl. In some embodiments, R³ is C₁-C₁₅ alkyl substituted with C₃-C₈ unsubstituted cycloalkyl. In some embodiments, R³ is

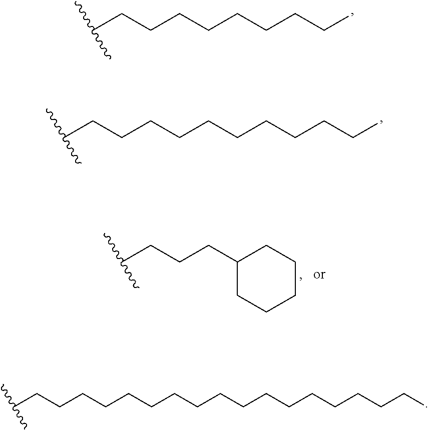

In some embodiments, R³ is

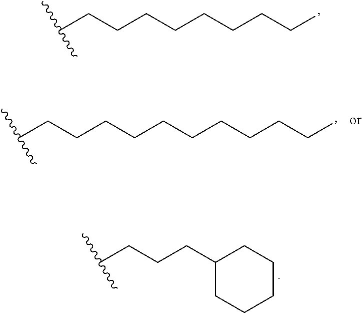

In some embodiments, R³ is

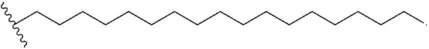

In some embodiments, R³ is C₁-C₂₅ optionally substituted alkenyl. In some embodiments, R³ is C₁-C₂₅ substituted alkenyl. In certain embodiments, R³ is C₁-C₂₅ unsubstituted alkenyl. In some embodiments, R³ is C₁-C₂₅ optionally substituted alkenyl comprising one double bond. In some embodiments, R³ is C₁-C₂₅ optionally substituted alkenyl comprising at least two double bonds. In some embodiments, R³ is

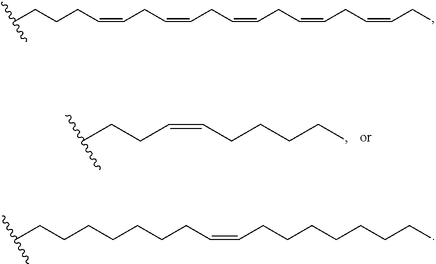

In some embodiments, R³ is

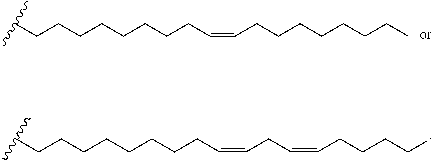

In some embodiments, R³ is

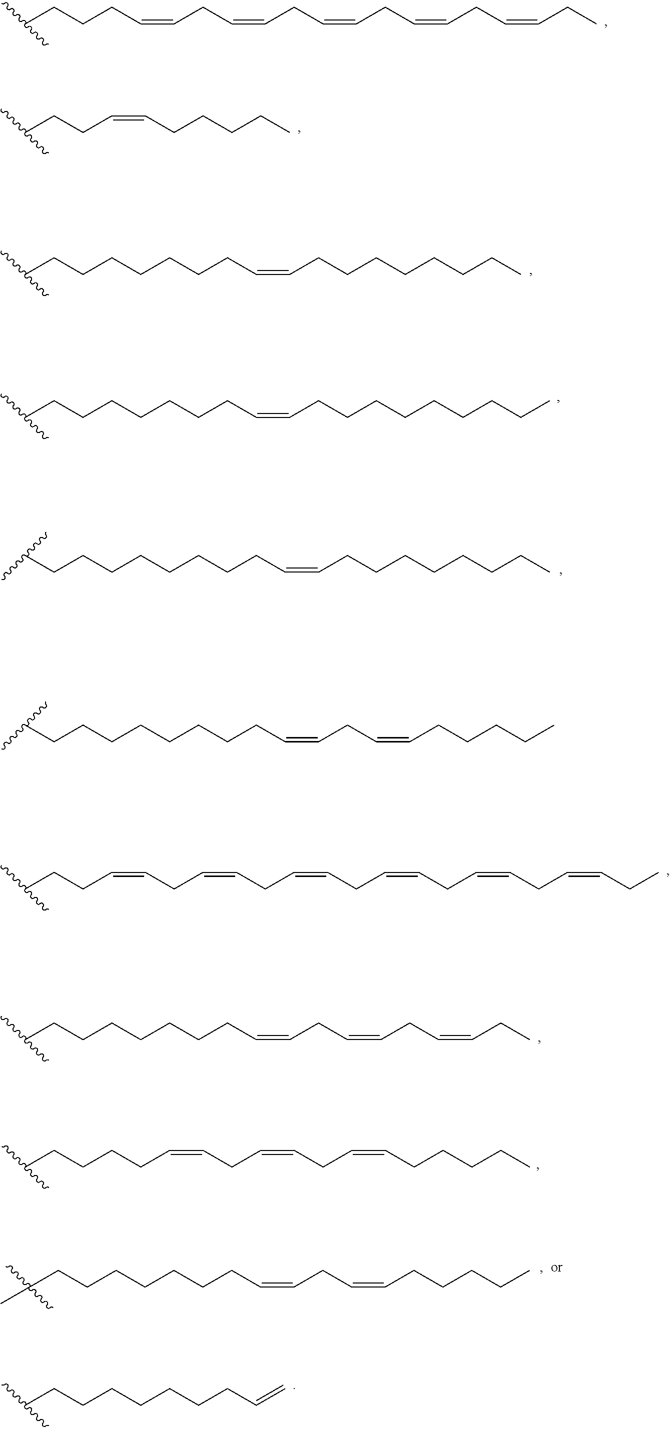

In some embodiments, R³ is C₁-C₂₅ optionally substituted alkynyl. In some embodiments, R³ is C₁-C₂₅ substituted alkynyl. In certain embodiments, R³ is C₁-C₂₅ unsubstituted alkynyl. In some embodiments, R³ is

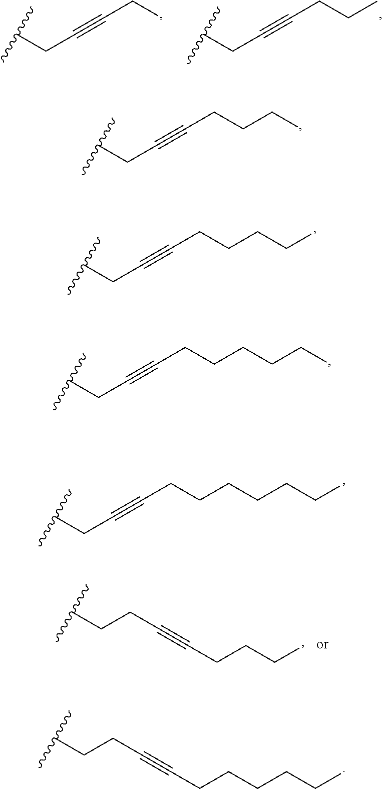

In certain embodiments, R³ is selected from the group consisting of

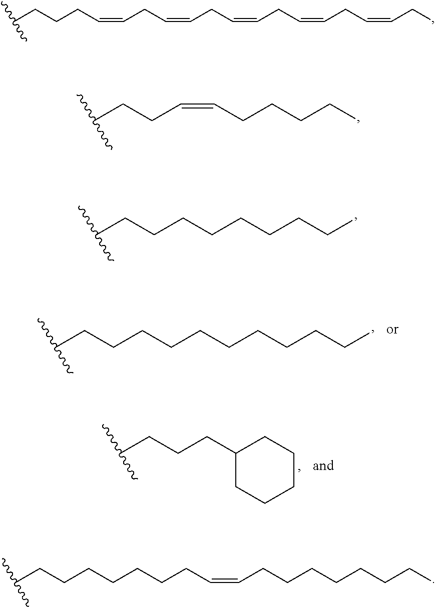

In certain embodiments, R³ is selected from the group consisting of

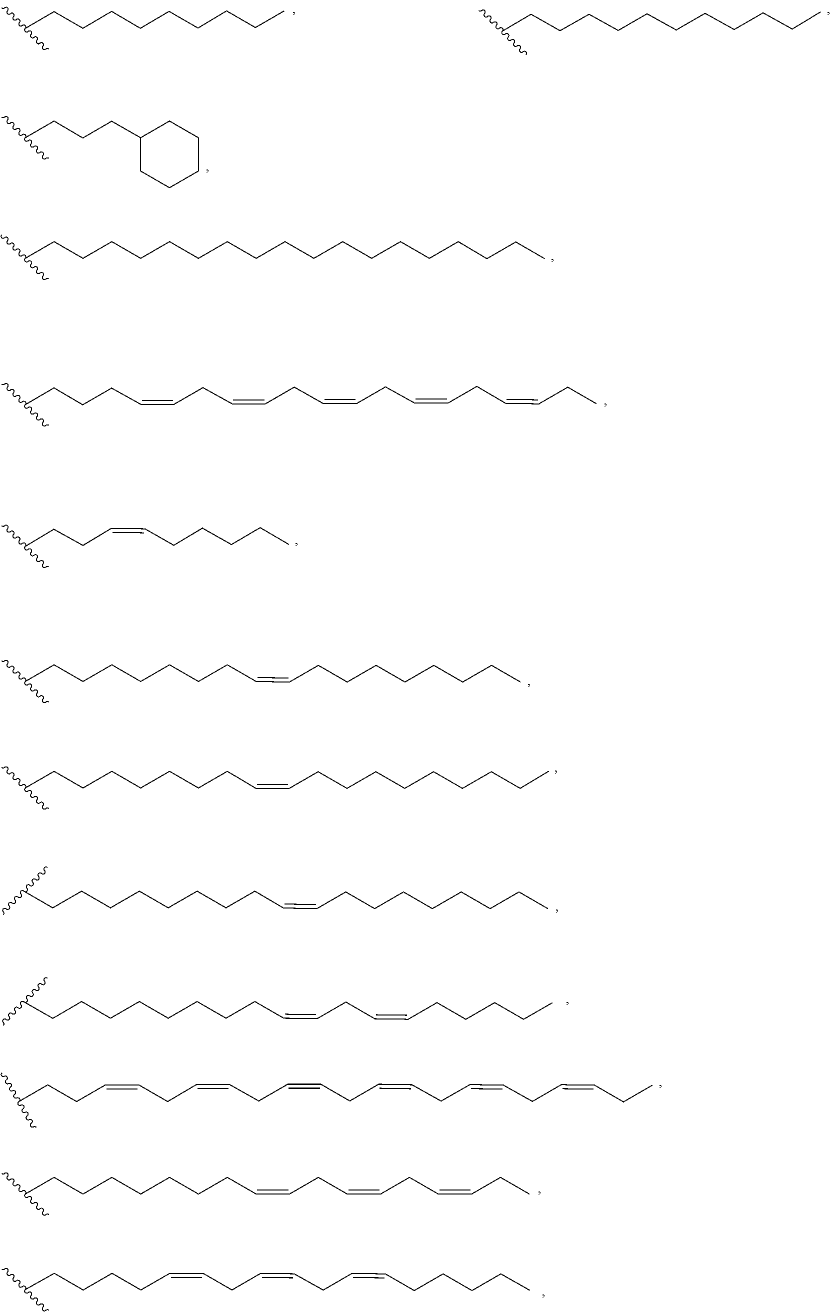

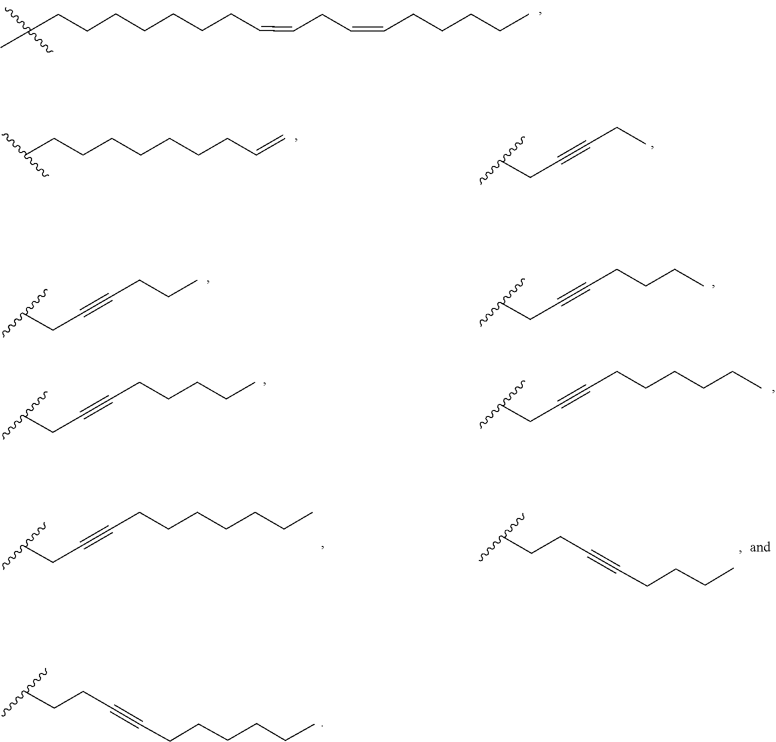

In certain embodiments, R³ is selected from the group consisting of

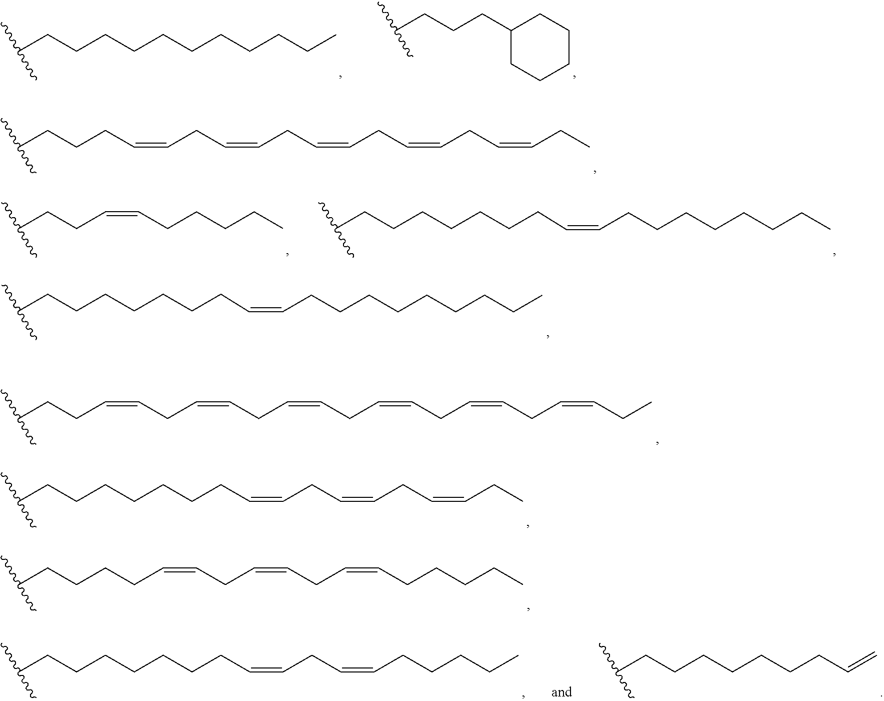

In some embodiments, R³ is optionally substituted C₁-C₂₅ heteroaliphatic. In certain embodiments, R³ is —OR^{3O} or —N(R^{3N})₂, wherein each instance of R^{3O} and R^{3N} is independently optionally substituted aliphatic. In certain embodiments, R³ is —OR^{3O} or —N(R^{3N})₂, wherein each instance of R^{3O} and R^{3N} is independently optionally substituted C₁-C₂₅ aliphatic.

In certain embodiments, R³ is —OR^{3O}, wherein R^{3O} is optionally substituted C₁-C₂₅ aliphatic.

In some embodiments, R^{3O} is C₁-C₂₅ optionally substituted aliphatic. In some embodiments, R^{3O} is C₁-C₂₅ substituted aliphatic. In certain embodiments, R^{3O} is C₁-C₂₅ unsubstituted aliphatic. In some embodiments, R^{3O} is C₁-C₂₅ optionally substituted alkyl. In some embodiments, R^{3O} is C₁-C₂₅ substituted alkyl. In certain embodiments, R^{3O} is C₁-C₂₅ unsubstituted alkyl. In some embodiments, R^{3O} is

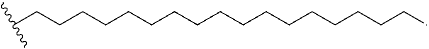

In some embodiments, R^{3O} is C₁-C₂₅ optionally substituted alkenyl. In some embodiments, R^{3O} is C₁-C₂₅ substituted alkenyl. In certain embodiments, R^{3O} is C₁-C₂₅ unsubstituted alkenyl. In some embodiments, R^{3O} is C₁-C₂₅ optionally substituted alkenyl comprising one double bond. In some embodiments, R^{3O} is C₁-C₂₅ optionally substituted alkenyl comprising at least two double bonds. In some embodiments, R^{3O} is

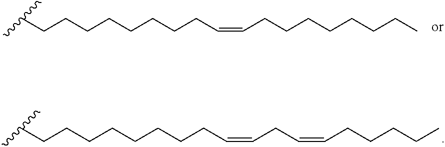 [SMILES: *CCCCCCCC/C=C\CCCCCCCC ; *CCCCCCCC/C=C\C/C=C\CCCCC]

In some embodiments, R^{3O} is C₁-C₂₅ optionally substituted alkynyl. In some embodiments, R^{3O} is C₁-C₂₅ substituted alkynyl. In certain embodiments, R^{3O} is C₁-C₂₅ unsubstituted alkynyl. In some embodiments, R^{3O} is

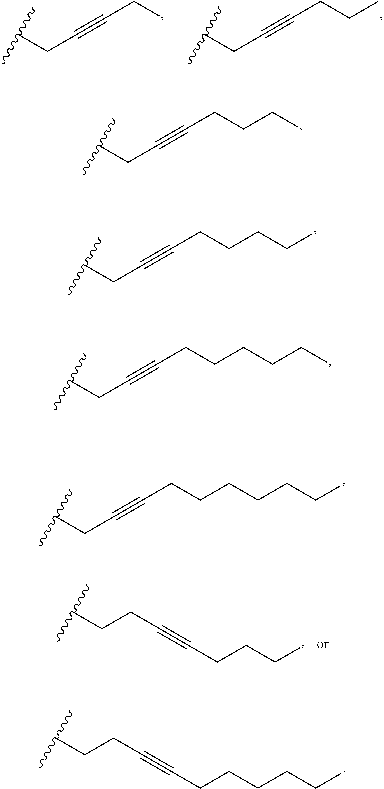

In some embodiments, R^{3O} is selected from the group consisting of

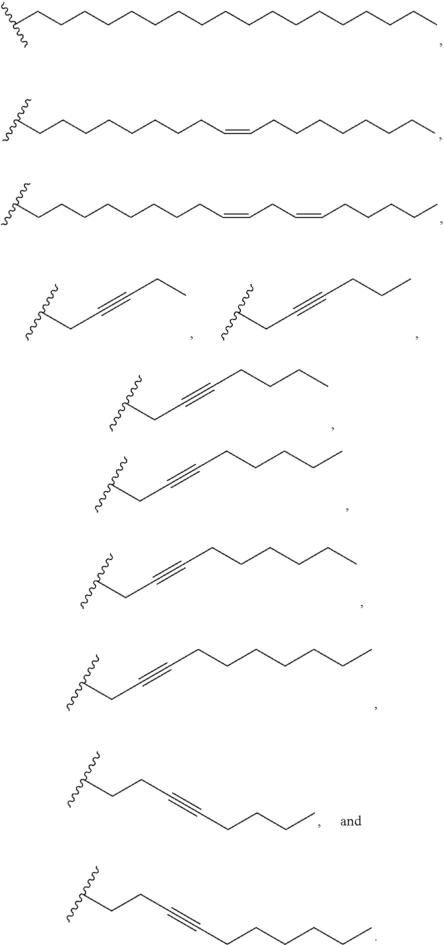

In certain embodiments, R³ is —N(R^{3N})₂, wherein each instance of R^{3N} is independently optionally substituted C₁-C₂₅ aliphatic.

In some embodiments, R^{3N} is C₁-C₂₅ optionally substituted aliphatic. In some embodiments, R^{3N} is C₁-C₂₅ substituted aliphatic. In certain embodiments, R^{3N} is C₁-C₂₅ unsubstituted aliphatic. In some embodiments, R^{3N} is C₁-C₂₅ optionally substituted alkyl. In some embodiments, R^{3N} is C₁-C₂₅ substituted alkyl.

In some embodiments, R^{3N} is C₁-C₂₅ optionally substituted alkenyl. In some embodiments, R^{3N} is C₁-C₂₅ substituted alkenyl. In certain embodiments, R^{3N} is C₁-C₂₅ unsubstituted alkenyl. In some embodiments, R^{3N} is C₁-C₂₅ optionally substituted alkenyl comprising one double bond. In some embodiments, R^{3N} is C₁-C₂₅ optionally substituted alkenyl comprising at least two double bonds.

In some embodiments, R^{3N} is C₁-C₂₅ optionally substituted alkynyl. In some embodiments, R^{3N} is C₁-C₂₅ substituted alkynyl. In certain embodiments, R^{3N} is C₁-C₂₅ unsubstituted alkynyl.

As defined herein, X is —OR¹, —SR¹, or —NR¹R². In certain embodiments, X is —OR¹, or —SR¹. In some embodiments, X is —SR¹ or —NR¹R². In certain embodiments, X is —OR¹ or —NR¹R². In some embodiments, X is —OR¹. In certain embodiments, X is —SR¹. In some embodiments, X is —NRR².

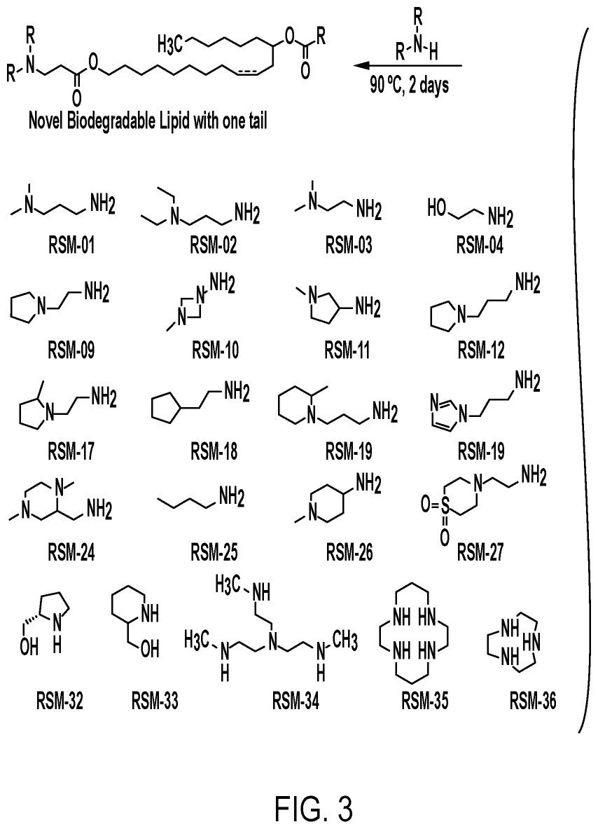

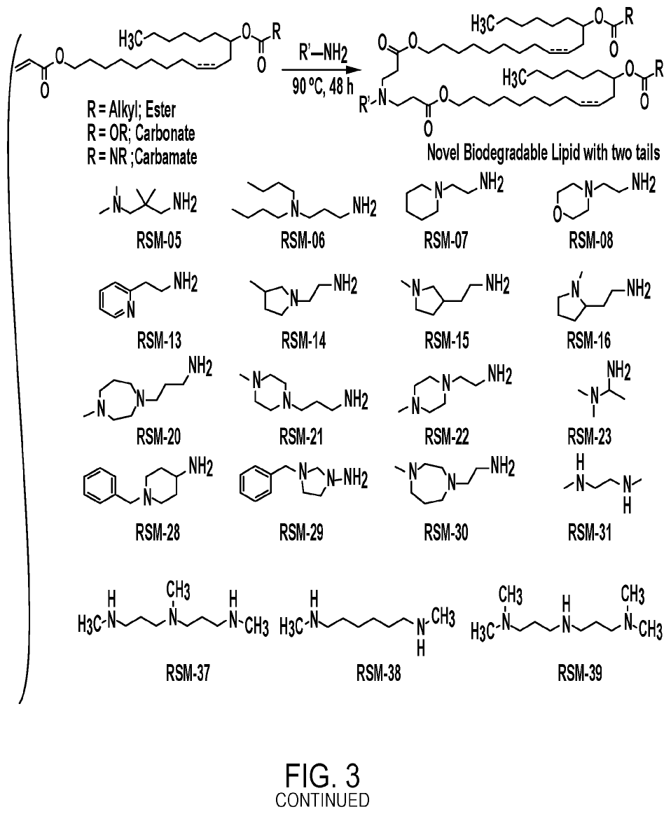

In some embodiments, H—X is H—NR¹R². In certain embodiments, H—X is selected from an amine provided in FIG. 3 or FIG. 10A.

As defined herein, R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group; or R¹ and R² are joined together with the intervening atoms to form an optionally substituted heterocyclyl.

In certain embodiments, R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group. In some embodiments, R¹ is —H, optionally substituted alkyl, optionally substituted heteroalkyl, optionally substituted aryl, optionally substituted heteroaryl, optionally substituted cycloalkyl, or optionally substituted heterocyclyl. In certain embodiments, R¹ is optionally substituted alkyl, optionally substituted heteroalkyl, optionally substituted aryl, optionally substituted heteroaryl, optionally substituted cycloalkyl, or optionally substituted heterocyclyl. In some embodiments, R¹ is —H, optionally substituted alkyl, optionally substituted heteroalkyl, optionally substituted cycloalkyl, or optionally substituted heterocyclyl. In some embodiments, R¹ is optionally substituted alkyl, optionally substituted heteroalkyl, optionally substituted cycloalkyl, or optionally substituted heterocyclyl. In certain embodiments, R¹ is —H, optionally substituted alkyl, optionally substituted heteroalkyl, or optionally substituted heterocyclyl. In certain embodiments, R¹ is optionally substituted alkyl, optionally substituted heteroalkyl, or optionally substituted heterocyclyl.

In certain embodiments, R¹ is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R¹ is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R¹ is optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In some embodiments, R¹ is optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In certain embodiments, R¹ is —H, optionally substituted C₁-C₂₅ alkyl, optionally substituted C₁-C₂₅ heteroalkyl, or optionally substituted 3- to 8-membered heterocyclyl. In certain embodiments, R¹ is optionally substituted C₁-C₂₅ alkyl, optionally substituted C₁-C₂₅ heteroalkyl, or optionally substituted 3- to 8-membered heterocyclyl.

In certain embodiments, R¹ is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R¹ is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R¹ is optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In some embodiments, R¹ is optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In certain embodiments, R¹ is —H, optionally substituted C₁-C₂₀ alkyl, optionally substituted C₁-C₂₀ heteroalkyl, or optionally substituted 3- to 8-membered heterocyclyl. In certain embodiments, R¹ is optionally substituted C₁-C₂₀ alkyl, optionally substituted C₁-C₂₀ heteroalkyl, or optionally substituted 3- to 8-membered heterocyclyl.

In some embodiments, R¹ is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃₋₈ cycloalkyl, optionally substituted 3- to 8-membered heterocyclyl, or a protecting group. In some embodiments, R¹ is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-s cycloalkyl, or optionally substituted 3- to 8-membered heterocyclyl. In certain embodiments, R¹ is optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In some embodiments, R¹ is optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In certain embodiments, R¹ is —H, optionally substituted C₁-C₁₀ alkyl, optionally substituted C₁-C₁₀ heteroalkyl, or optionally substituted 3- to 8-membered heterocyclyl. In certain embodiments, R¹ is optionally substituted C₁-C₁₀ alkyl, optionally substituted C₁-C₁₀ heteroalkyl, or optionally substituted 3- to 8-membered heterocyclyl.

In some embodiments, R¹ is a protecting group. In some embodiments the protecting group is a nitrogen protecting group, oxygen protecting group, or sulfur protecting group. In some embodiments, the protecting group is a nitrogen protecting group. In some embodiments the protecting group is an oxygen protecting group. In some embodiments, the protecting group is a sulfur protecting group. In some embodiments, R¹ is —H. In certain embodiments, R¹ is —H or a protecting group. In some embodiments, R¹ is —H or a nitrogen protecting group, an oxygen protecting group, or a sulfur protecting group. In some embodiments, R¹ is —H or a nitrogen protecting group.

In certain embodiments, R¹ is optionally substituted alkyl. In some embodiments, R¹ is optionally substituted C₁-C₂₅ alkyl. In certain embodiments, R¹ is optionally substituted C₁-C₂₀ alkyl. In some embodiments, R¹ is optionally substituted C₁-C₁₅ alkyl. In certain embodiments, R¹ is optionally substituted C₁-C₁₀ alkyl. In some embodiments, R¹ is optionally substituted C₁-C₆ alkyl. In certain embodiments, R¹ is substituted alkyl. In some embodiments, R¹ is substituted C₁-C₂₅ alkyl. In certain embodiments, R¹ is substituted C₁-C₂₀ alkyl. In some embodiments, R¹ is substituted C₁-C₁₅ alkyl. In certain embodiments, R¹ is substituted C₁-C₁₀ alkyl. In some embodiments, R¹ is substituted C₁-C₆ alkyl. In certain embodiments, R¹ is unsubstituted alkyl. In some embodiments, R¹ is unsubstituted C₁-C₂₅ alkyl. In certain embodiments, R¹ is unsubstituted C₁-C₂₀ alkyl. In some embodiments, R¹ is unsubstituted C₁-C₁₅ alkyl. In certain embodiments, R¹ is unsubstituted C₁-C₁₀ alkyl. In some embodiments, R¹ is unsubstituted C₁-C₆ alkyl. In certain embodiments, R¹ is methyl, ethyl, propyl, or butyl. In some embodiments, R¹ is methyl or ethyl. In certain embodiments, R¹ is methyl. In some embodiments, R¹ is ethyl.

In certain embodiments, R¹ is optionally substituted heteroalkyl. In some embodiments, R¹ is optionally substituted C₁-C₂₅ heteroalkyl. In certain embodiments, R¹ is optionally substituted C₁-C₂₀ heteroalkyl. In some embodiments, R¹ is optionally substituted C₁-C₁₅ heteroalkyl. In certain embodiments, R¹ is optionally substituted C₁-C₁₀ heteroalkyl. In some embodiments, R¹ is optionally substituted C₁-C₆ heteroalkyl. In certain embodiments, R¹ is substituted heteroalkyl. In some embodiments, R¹ is substituted C₁-C₂₅ heteroalkyl. In certain embodiments, R¹ is substituted C₁-C₂₀ heteroalkyl. In some embodiments, R¹ is substituted C₁-C₁₅ heteroalkyl. In certain embodiments, R¹ is substituted C₁-C₁₀ heteroalkyl. In some embodiments, R¹ is substituted C₁-C₆ heteroalkyl. In certain embodiments, R¹ is unsubstituted heteroalkyl. In some embodiments, R¹ is unsubstituted C₁-C₂₅ heteroalkyl. In certain embodiments, R¹ is unsubstituted C₁-C₂₀ heteroalkyl. In some embodiments, R¹ is unsubstituted C₁-C₁₅ heteroalkyl. In certain embodiments, R¹ is unsubstituted C₁-C₁₀ heteroalkyl. In some embodiments, R¹ is unsubstituted C₁-C₆ heteroalkyl. In some embodiments, R¹ is

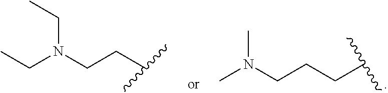

In some embodiments, R¹ is optionally substituted C₆-C₁₄ aryl. In some embodiments, R¹ is substituted C₆-C₁₄ aryl. In some embodiments, R¹ is unsubstituted C₆-C₁₄ aryl. In certain embodiments, R¹ is optionally substituted C₆-C₁₀ aryl. In certain embodiments, R¹ is substituted C₆-C₁₀ aryl. In certain embodiments, R¹ is unsubstituted C₆-C₁₀ aryl. In some embodiments, R¹ is optionally substituted phenyl or optionally substituted naphthyl. In certain embodiments, R¹ is optionally substituted phenyl. In some embodiments, R¹ is optionally substituted naphthyl.

In some embodiments, R¹ is optionally substituted 5- to 14-membered heteroaryl. In some embodiments, R¹ is substituted 5- to 14-membered heteroaryl. In some embodiments, R¹ is unsubstituted 5- to 14-membered heteroaryl. In some embodiments, R¹ is optionally substituted 5- to 10-membered heteroaryl. In some embodiments, R¹ is substituted 5- to 10-membered heteroaryl. In some embodiments, R¹ is unsubstituted 5- to 10-membered heteroaryl. In some embodiments, R¹ is optionally substituted pyridinyl.

In some embodiments, R¹ is optionally substituted C₃-C₈ cycloalkyl. In some embodiments, R¹ is substituted C₃-C₈ cycloalkyl. In some embodiments, R¹ is unsubstituted C₃-C₈ cycloalkyl. In some embodiments, R¹ is C₃-C₈ cycloalkyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R¹ is optionally substituted 3- to 8-membered heterocyclyl. In some embodiments, R¹ is substituted 3- to 8-membered heterocyclyl. In some embodiments, R¹ is unsubstituted 3- to 8-membered heterocyclyl. In some embodiments, R¹ is 3- to 8-membered heterocyclyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R¹ is not

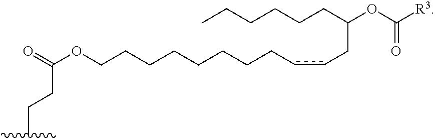

In certain embodiments, R¹ is —H, -Me, or -Et. In some embodiments, R¹ is —H, -Me, -Et,

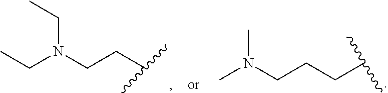

In certain embodiments, R¹ is —H, -Me, or -Et,

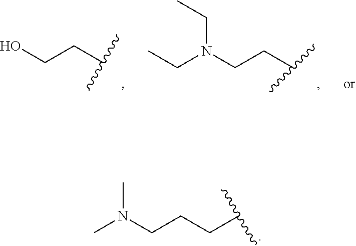

In certain embodiments, R¹ is —H, -Me, or -Et,

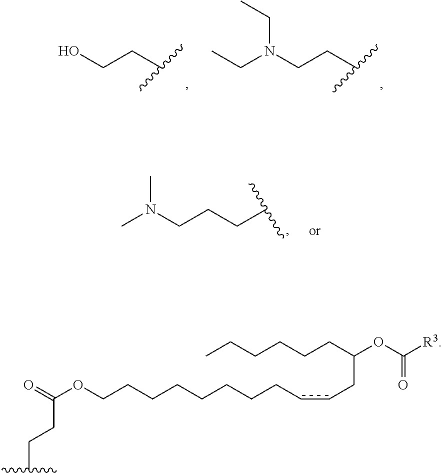

In some embodiments, R¹ is —H, -Me, or -Et,

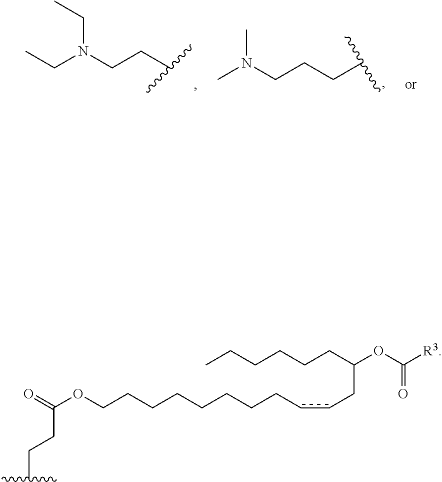

In some embodiments, R¹ is —H, -Me, -Et, or

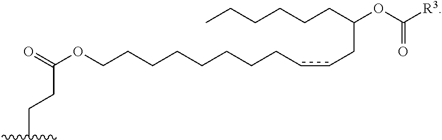

In some embodiments, R¹ is

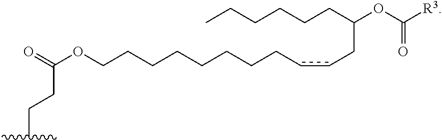

As defined herein, R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or R¹ and R² are joined together with the intervening atoms to form an optionally substituted heterocyclyl.

In certain embodiments, R² is —H, optionally substituted alkyl, optionally substituted heteroalkyl, optionally substituted aryl, optionally substituted heteroaryl, optionally substituted cycloalkyl, optionally substituted heterocyclyl, or a protecting group. In some embodiments, R² is —H, optionally substituted alkyl, optionally substituted heteroalkyl, optionally substituted aryl, optionally substituted heteroaryl, optionally substituted cycloalkyl, or optionally substituted heterocyclyl. In certain embodiments, R² is optionally substituted alkyl, optionally substituted heteroalkyl, optionally substituted aryl, optionally substituted heteroaryl, optionally substituted cycloalkyl, or optionally substituted heterocyclyl. In some embodiments, R² is —H, optionally substituted alkyl, optionally substituted heteroalkyl, optionally substituted cycloalkyl, or optionally substituted heterocyclyl. In some embodiments, R² is optionally substituted alkyl, optionally substituted heteroalkyl, optionally substituted cycloalkyl, or optionally substituted heterocyclyl. In certain embodiments, R² is —H, optionally substituted alkyl, optionally substituted heteroalkyl, or optionally substituted heterocyclyl. In certain embodiments, R² is optionally substituted alkyl, optionally substituted heteroalkyl, or optionally substituted heterocyclyl.

In certain embodiments, R² is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R² is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R² is optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In some embodiments, R² is optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In certain embodiments, R² is —H, optionally substituted C₁-C₂₅ alkyl, optionally substituted C₁-C₂₅ heteroalkyl, or optionally substituted 3- to 8-membered heterocyclyl. In certain embodiments, R² is optionally substituted C₁-C₂₅ alkyl, optionally substituted C₁-C₂₅ heteroalkyl, or optionally substituted 3- to 8-membered heterocyclyl.

In certain embodiments, R² is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R² is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R² is optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In some embodiments, R² is optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In certain embodiments, R² is —H, optionally substituted C₁-C₂₀ alkyl, optionally substituted C₁-C₂₀ heteroalkyl, or optionally substituted 3- to 8-membered heterocyclyl. In certain embodiments, R² is optionally substituted C₁-C₂₀ alkyl, optionally substituted C₁-C₂₀ heteroalkyl, or optionally substituted 3- to 8-membered heterocyclyl.

In some embodiments, R² is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃₋₈ cycloalkyl, optionally substituted 3- to 8-membered heterocyclyl, or a protecting group. In some embodiments, R² is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-s cycloalkyl, or optionally substituted 3- to 8-membered heterocyclyl. In certain embodiments, R² is optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In some embodiments, R² is optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In certain embodiments, R² is —H, optionally substituted C₁-C₁₀ alkyl, optionally substituted C₁-C₁₀ heteroalkyl, or optionally substituted 3- to 8-membered heterocyclyl. In certain embodiments, R² is optionally substituted C₁-C₁₀ alkyl, optionally substituted C₁-C₁₀ heteroalkyl, or optionally substituted 3- to 8-membered heterocyclyl.

In some embodiments, R² is a protecting group. In some embodiments the protecting group is a nitrogen protecting group. In some embodiments, R² is —H. In some embodiments, R² is —H or a protecting group. In some embodiments, R² is —H or a nitrogen protecting group.

In certain embodiments, R² is optionally substituted alkyl. In some embodiments, R² is optionally substituted C₁-C₂₅ alkyl. In certain embodiments, R² is optionally substituted C₁-C₂₀ alkyl. In some embodiments, R² is optionally substituted C₁-C₁₅ alkyl. In certain embodiments, R² is optionally substituted C₁-C₁₀ alkyl. In some embodiments, R² is optionally substituted C₁-C₆ alkyl. In certain embodiments, R² is substituted alkyl. In some embodiments, R² is substituted C₁-C₂₅ alkyl. In certain embodiments, R² is substituted C₁-C₂₀ alkyl. In some embodiments, R² is substituted C₁-C₁₅ alkyl. In certain embodiments, R² is substituted C₁-C₁₀ alkyl. In some embodiments, R² is substituted C₁-C₆ alkyl. In certain embodiments, R² is unsubstituted alkyl. In some embodiments, R² is unsubstituted C₁-C₂₅ alkyl. In certain embodiments, R² is unsubstituted C₁-C₂₀ alkyl. In some embodiments, R² is unsubstituted C₁-C₁₅ alkyl. In certain embodiments, R² is unsubstituted C₁-C₁₀ alkyl. In some embodiments, R² is unsubstituted C₁-C₆ alkyl. In certain embodiments, R² is methyl, ethyl, propyl, or butyl. In some embodiments, R² is methyl or ethyl. In certain embodiments, R² is methyl. In some embodiments, R² is ethyl.

In certain embodiments, R² is optionally substituted heteroalkyl. In some embodiments, R² is optionally substituted C₁-C₂₅ heteroalkyl. In certain embodiments, R² is optionally substituted C₁-C₂₀ heteroalkyl. In some embodiments, R² is optionally substituted C₁-C₁₅ heteroalkyl. In certain embodiments, R² is optionally substituted C₁-C₁₀ heteroalkyl. In some embodiments, R² is optionally substituted C₁-C₆ heteroalkyl. In certain embodiments, R² is substituted heteroalkyl. In some embodiments, R² is substituted C₁-C₂₅ heteroalkyl. In certain embodiments, R² is substituted C₁-C₂₀ heteroalkyl. In some embodiments, R² is substituted C₁-C₁₅ heteroalkyl. In certain embodiments, R² is substituted C₁-C₁₀ heteroalkyl. In some embodiments, R² is substituted C₁-C₆ heteroalkyl. In certain embodiments, R² is unsubstituted heteroalkyl. In some embodiments, R² is unsubstituted C₁-C₂₅ heteroalkyl. In certain embodiments, R² is unsubstituted C₁-C₂₀ heteroalkyl. In some embodiments, R² is unsubstituted C₁-C₁₅ heteroalkyl. In certain embodiments, R² is unsubstituted C₁-C₁₀ heteroalkyl. In some embodiments, R² is unsubstituted C₁-C₆ heteroalkyl.

In certain embodiments, R² is optionally substituted heteroalkyl comprising one or more N atoms. In some embodiments, R² is optionally substituted heteroalkyl, wherein the optionally substituted alkyl comprises two N atoms. In certain embodiments, R² is optionally substituted heteroalkyl, wherein the optionally substituted alkyl comprises three N atoms. In certain embodiments, R² is

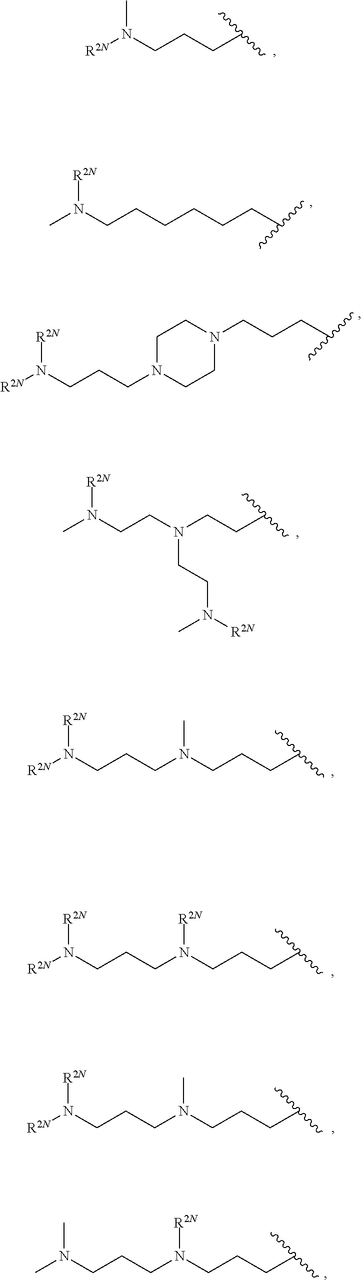

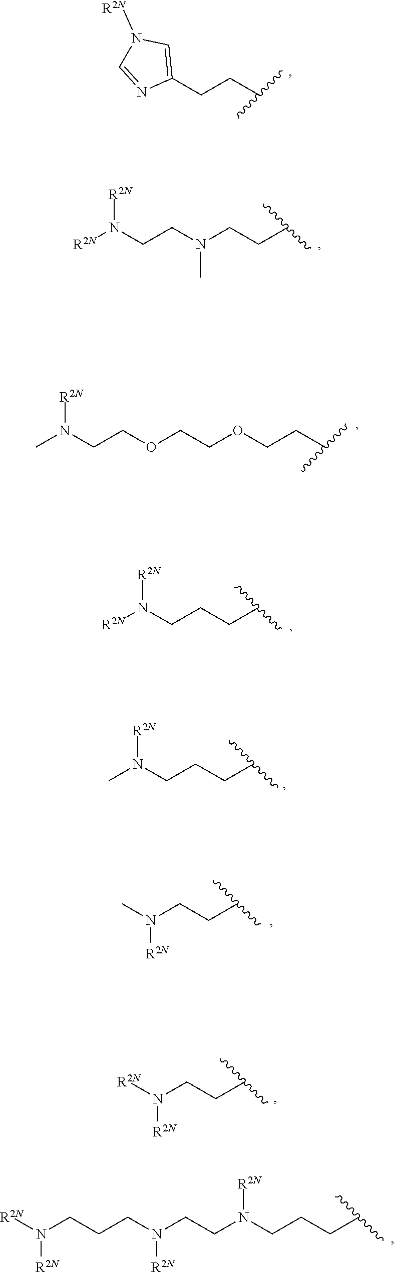

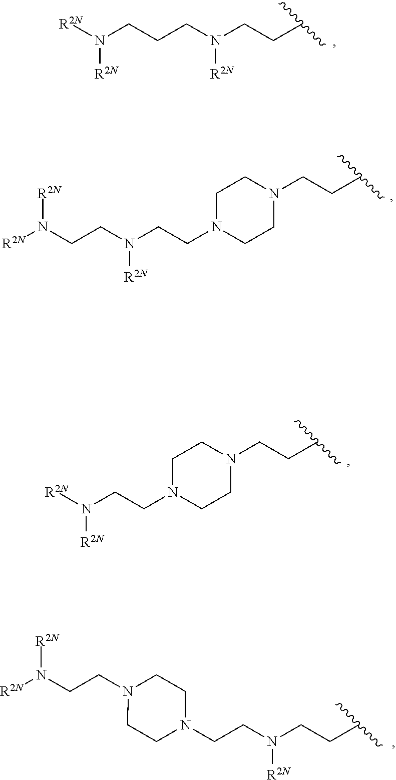

wherein each instance of R^{2N} is —H. In certain embodiments, R² is

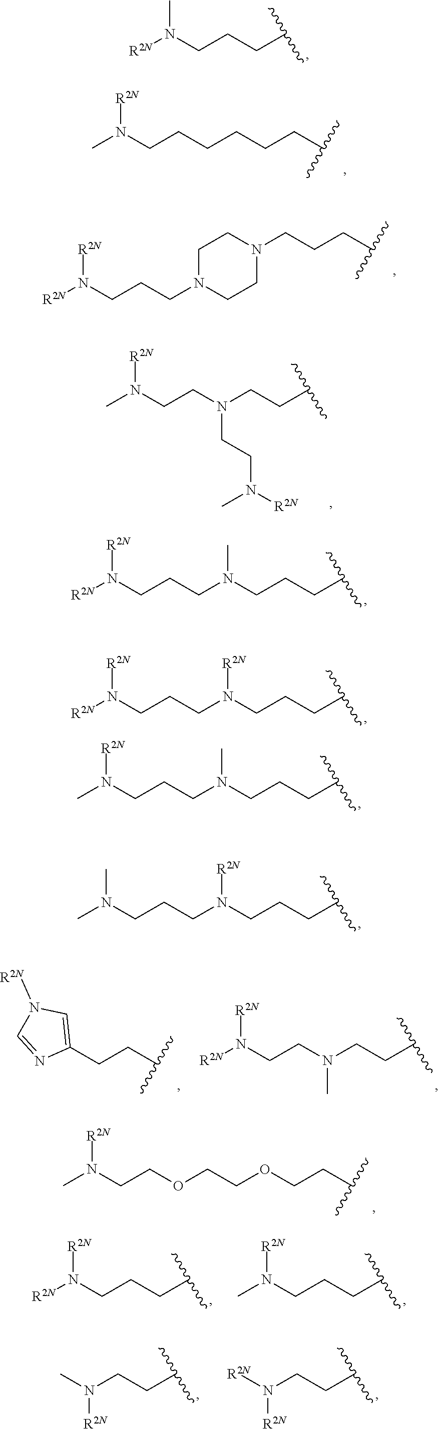

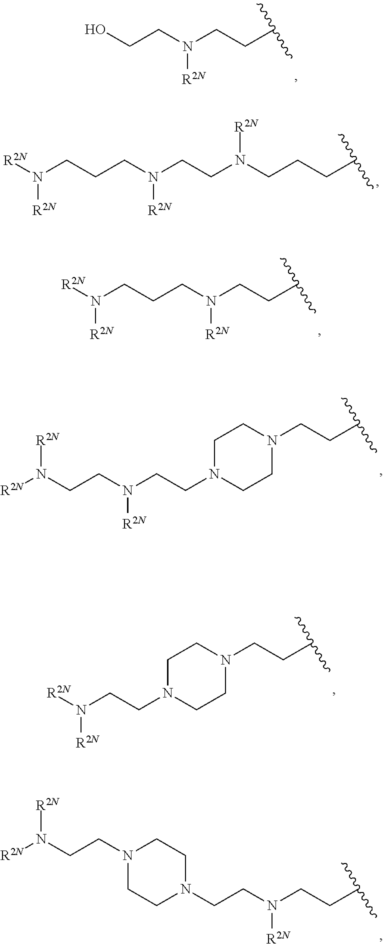

wherein each instance of R^{2N} is independently —H, optionally substituted C₁₋₁₀ aliphatic, optionally substituted C₁₋₁₀ heteroaliphatic, optionally substituted acyl, or a nitrogen protecting group. In certain embodiments, R² is

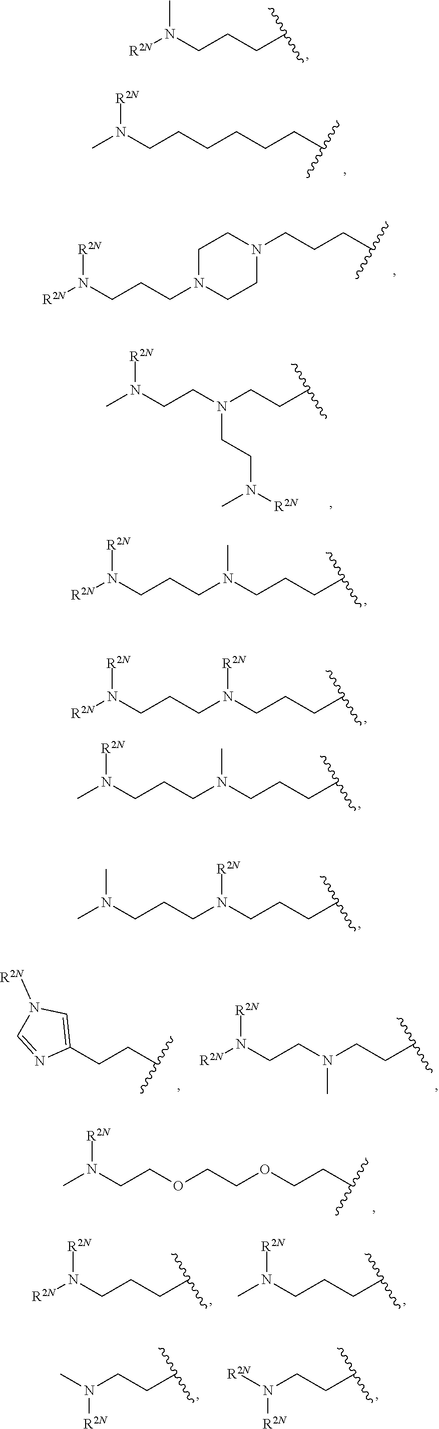

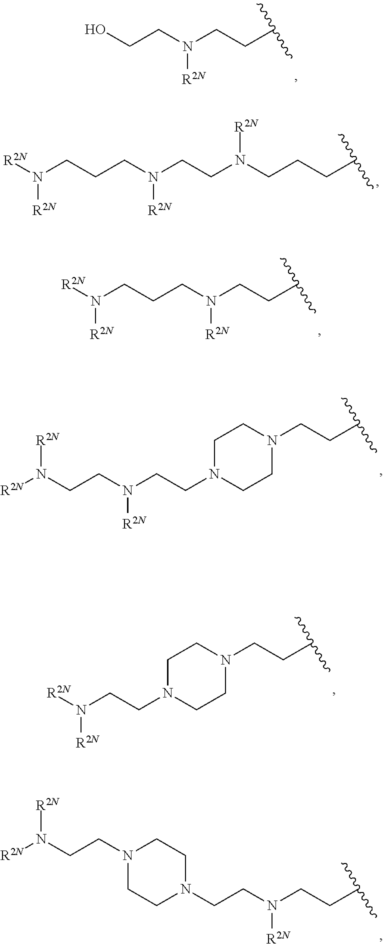

wherein each instance of R^{2N} is independently —H, optionally substituted C₁₋₁₀ aliphatic, optionally substituted C₁₋₁₀ heteroaliphatic, optionally substituted acyl, a nitrogen protecting group, or

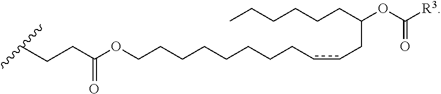

In some embodiments, R^{2N} is —H or

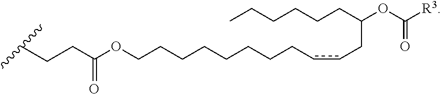

In some embodiments, R^{2N} is —H. In certain embodiments, R^{2N} is

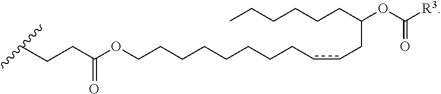

In some embodiments, R^{2N} is optionally substituted acyl. In some embodiments, R^{2N} is a nitrogen protecting group. In some embodiments, R^{2N} is Bn, Boc, Cbz, Fmoc, trifluoroacetyl, triphenylmethyl, acetyl, or Ts. In some embodiments, R^{2N} is optionally substituted C₁₋₁₀ aliphatic or optionally substituted C₁₋₁₀ heteroaliphatic. In some embodiments, R^{2N} is optionally substituted C₁₋₁₀ aliphatic. In some embodiments, R^{2N} is C₁₋₁₀ alkyl. In some embodiments, R^{2N} is -Me, -Et, -nPr, -iPr, -nBu, -iBu, or -tBu. In some embodiments, R^{2N} is -Me or -Et. In some embodiments, R^{2N} is -Me. In some embodiments, R^{2N} is -Et. In some embodiments, R^{2N} is C₂₋₁₀ alkenyl. In some embodiments, R^{2N} is C₂₋₁₀ alkynyl. In some embodiments, R^{2N} is optionally substituted C₁₋₁₀ heteroaliphatic. In some embodiments, R^{2N} is C₁₋₁₀ heteroalkyl. In some embodiments, R^{2N} is C₂₋₁₀ heteroalkenyl. In some embodiments, R^{2N} is C₂₋₁₀ heteroalkynyl

In some embodiments, R² is optionally substituted C₆-C₁₄ aryl. In some embodiments, R² is substituted C₆-C₁₄ aryl. In some embodiments, R² is unsubstituted C₆-C₁₄ aryl. In certain embodiments, R² is optionally substituted C₆-C₁₀ aryl. In certain embodiments, R² is substituted C₆-C₁₀ aryl. In certain embodiments, R² is unsubstituted C₆-C₁₀ aryl. In some embodiments, R² is optionally substituted phenyl or optionally substituted naphthyl. In certain embodiments, R² is optionally substituted phenyl. In some embodiments, R² is optionally substituted naphthyl.

In some embodiments, R² is optionally substituted 5- to 14-membered heteroaryl. In some embodiments, R² is substituted 5- to 14-membered heteroaryl. In some embodiments, R² is unsubstituted 5- to 14-membered heteroaryl. In some embodiments, R² is optionally substituted 5- to 10-membered heteroaryl. In some embodiments, R² is substituted 5- to 10-membered heteroaryl. In some embodiments, R² is unsubstituted 5- to 10-membered heteroaryl. In some embodiments, R² is optionally substituted pyridinyl.

In some embodiments, R² is optionally substituted C₃-C₈ cycloalkyl. In some embodiments, R² is substituted C₃-C₈ cycloalkyl. In some embodiments, R² is unsubstituted C₃-C₈ cycloalkyl. In some embodiments, R² is C₃-C₈ cycloalkyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R² is optionally substituted 3- to 8-membered heterocyclyl. In some embodiments, R² is substituted 3- to 8-membered heterocyclyl. In some embodiments, R² is unsubstituted 3- to 8-membered heterocyclyl. In some embodiments, R² is 3- to 8-membered heterocyclyl substituted with optionally substituted alkyl, optionally substituted heteroalkyl, optionally substituted cycloalkyl, optionally substituted heterocyclyl, optionally substituted heteroaryl, or optionally substituted aryl.

In certain embodiments, R² is selected from the group consisting of -Me,

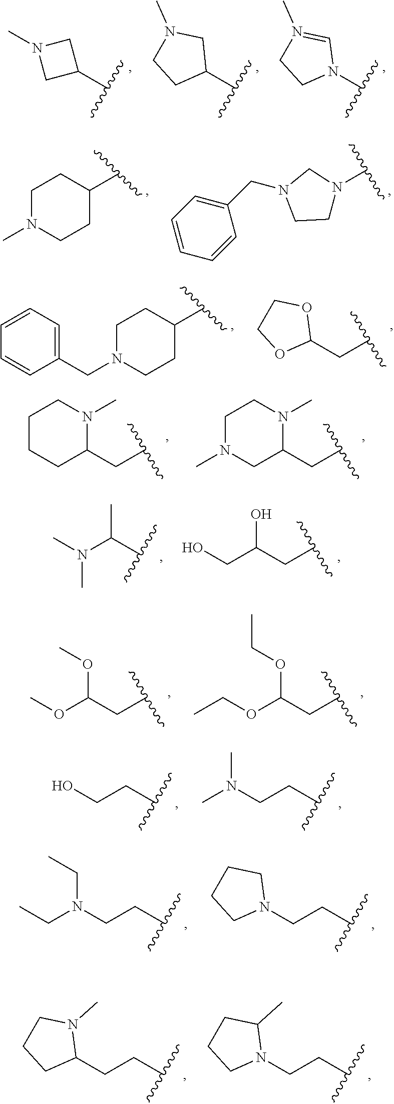

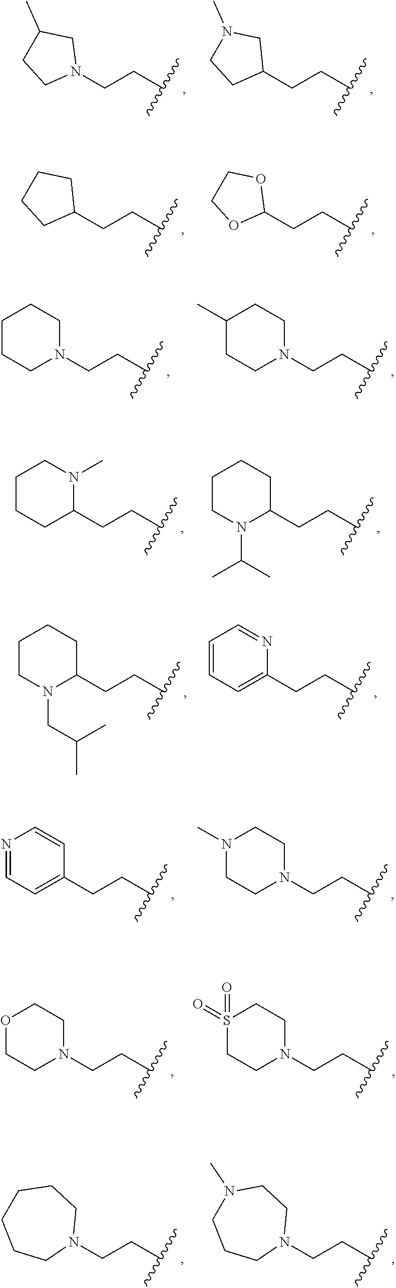

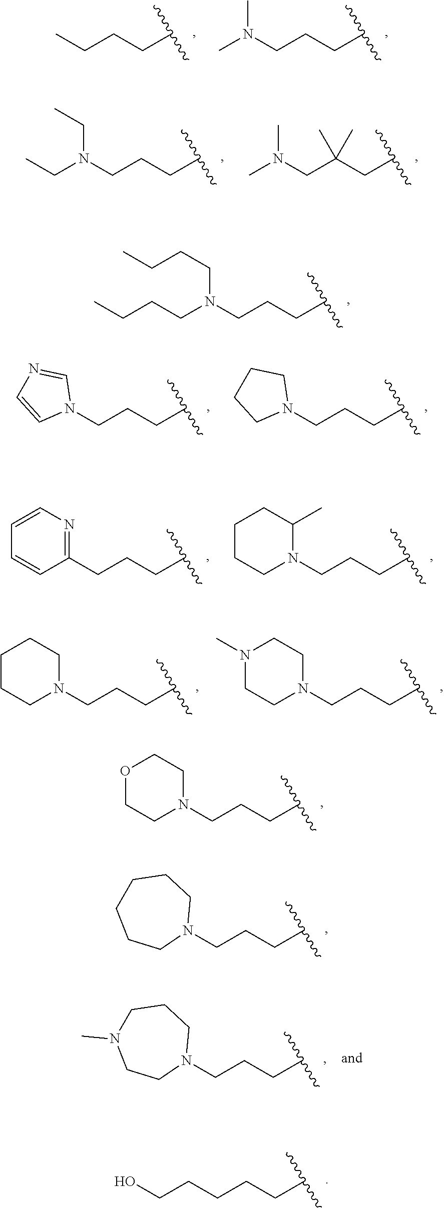

In certain embodiments, R² is selected from the group consisting of -Me,

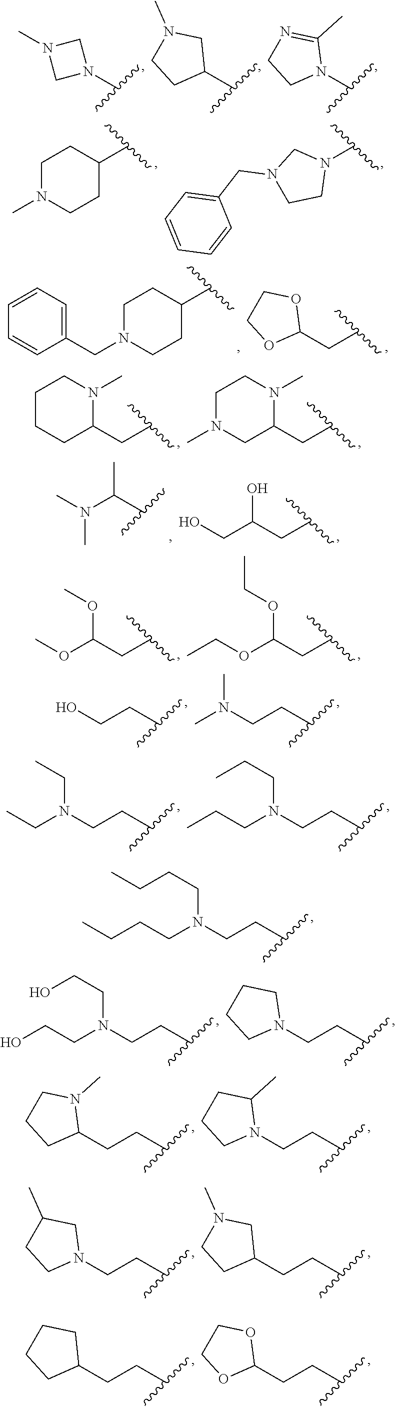

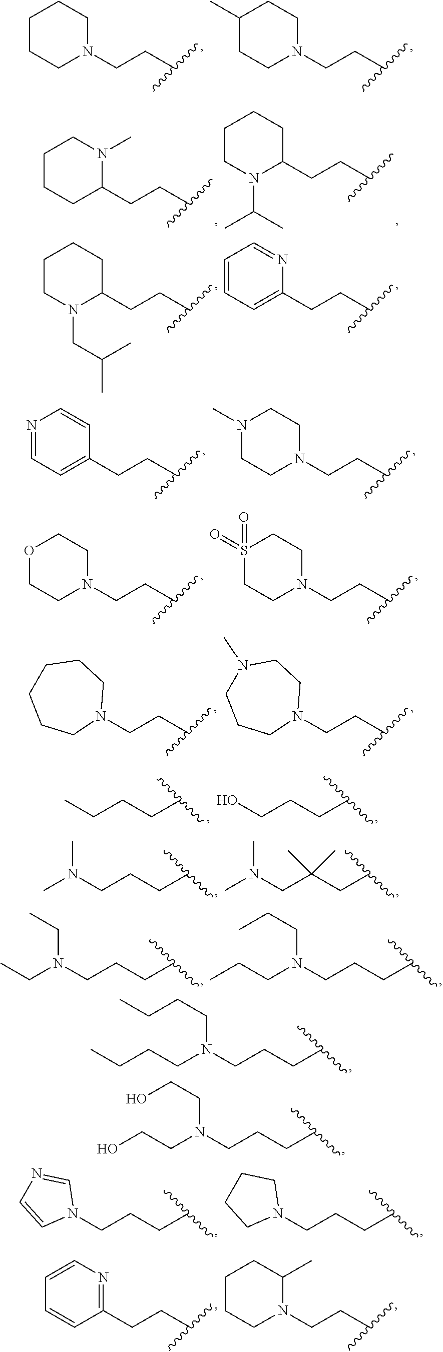

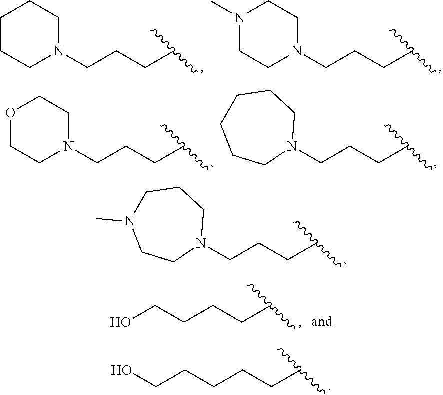

In some embodiments, R¹ is not —H. In certain embodiments, R² is not —H. In some embodiments, R¹ and R² are not both H.

In certain embodiments, R¹ and R² are joined together with the intervening atoms to form an optionally substituted heterocycle. In some embodiments, R¹ and R² are joined together with the intervening atoms to form an optionally substituted N-heterocycle. In certain embodiments, R¹ and R² are joined together with the intervening atoms to form an optionally substituted 3- to 6-membered heterocycle. In some embodiments, R¹ and R² are joined together with the intervening atoms to form an optionally substituted 3- to 6-membered N-heterocycle. In some embodiments, R¹ and R² are joined together with the intervening atoms to form a substituted 3- to 6-membered N-heterocycle. In some embodiments, R¹ and R² are joined together with the intervening atoms to form an unsubstituted 3- to 6-membered N-heterocycle. In certain embodiments, R¹ and R² are joined together with the intervening atoms to form optionally substituted aziridine, azetidine, pyrrolidine, piperidine, or piperazine. In certain embodiments, R¹ and R² are joined together with the intervening atoms to form aziridine, azetidine, pyrrolidine, piperidine, or piperazine substituted with C₁-C₁₀ alkyl, C₁-C₁₀ heteroalkyl, C₃-C₈ carbocyclyl, 3- to 8-membered heterocyclyl, C₆-C₁₄ aryl, or 5- to 14-membered heteroaryl. In certain embodiments, R¹ and R² are joined together with the intervening atoms to form unsubstituted aziridine, azetidine, pyrrolidine, piperidine, or piperazine.

In certain embodiments, X is —NR¹R² wherein R¹ and R² are joined together with the intervening atoms to form an optionally substituted heterocycle selected from the group consisting of

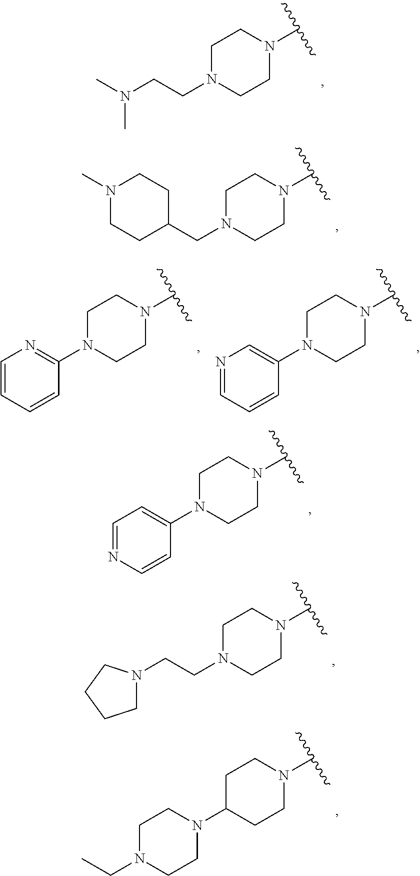

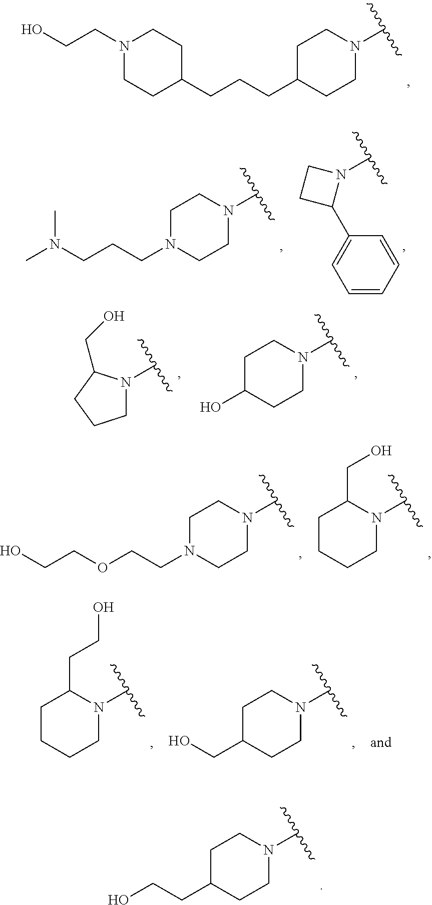

In certain embodiments, X is —NR¹R² wherein R¹ and R² are joined together with the intervening atoms to form an optionally substituted heterocycle selected from the group consisting of

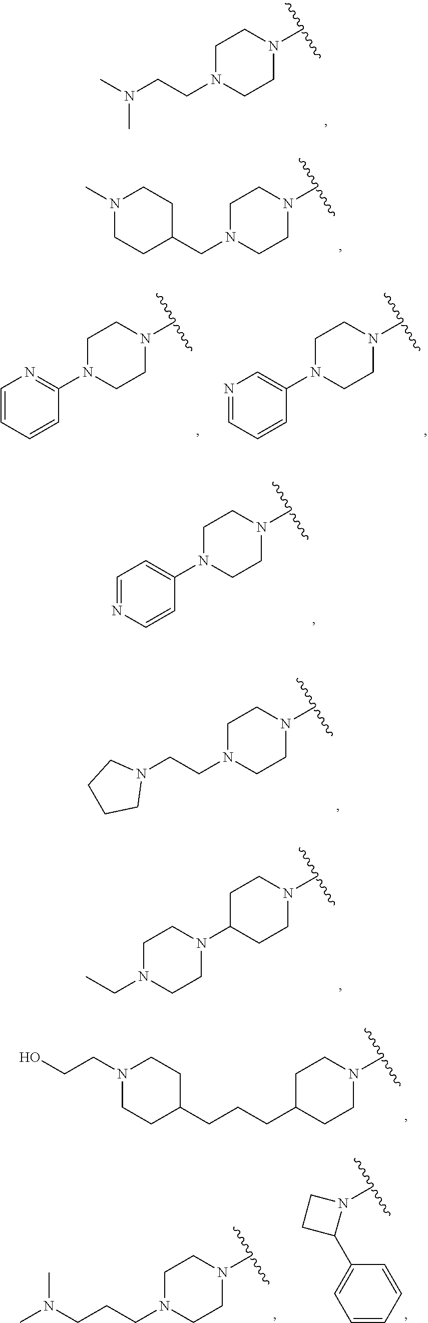

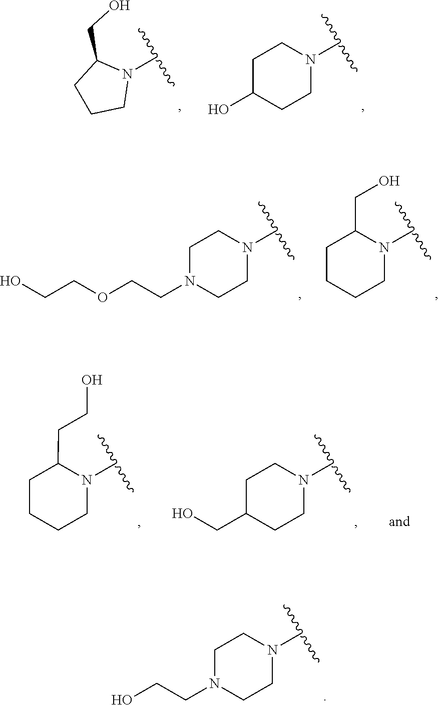

In some embodiments, R¹ and R² are joined together with the intervening atoms to form an optionally substituted heterocycle comprising one or more N atoms. In certain embodiments, R¹ and R² are joined together with the intervening atoms to form an optionally substituted heterocycle comprising two N atoms. In some embodiments, R¹ and R² are joined together with the intervening atoms to form an optionally substituted heterocycle comprising three N atoms. In some embodiments, R¹ and R² are joined together with the intervening atoms to form an optionally substituted heterocycle selected from the group consisting of:

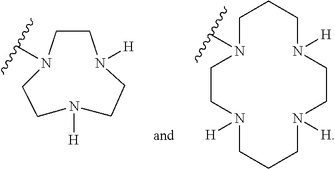

In certain embodiments,

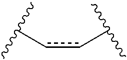

represents a single bond. In some embodiments,

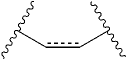

represents a double bond.

### Pharmaceutical Compositions and Administration

The present disclosure provides compositions (e.g., pharmaceutical compositions) comprising a compound provided herein (e.g., a compound of Formula (I) or (II)), or a pharmaceutically acceptable salt, solvate, tautomer, stereoisomer, or isotopically labeled derivative thereof, and an excipient (e.g., a pharmaceutically acceptable excipient). In one aspect, the composition is a pharmaceutical composition, and the excipient is a pharmaceutically acceptable excipient.

In certain embodiments, the compound described herein is provided in an effective amount in the pharmaceutical composition. In certain embodiments, the effective amount is an amount effective for delivering an agent to a subject or cell. In certain embodiments, the effective amount is an amount effective for delivering a polynucleotide to a subject or cell. In certain embodiments, the effective amount is an amount effective for delivering mRNA to a subject or cell.

In certain embodiments, the pharmaceutical composition further comprises one or more of a PEG-lipid, sterol, phospholipid, or charged lipid. In some embodiments, the pharmaceutical composition comprises a PEG-lipid, sterol, phospholipid, and charged lipid. In certain embodiments, the pharmaceutical composition comprises a PEG-lipid, sterol, and phospholipid. In some embodiments, the pharmaceutical composition comprises a PEG-lipid, sterol, and charged lipid. In certain embodiments, the pharmaceutical composition comprises a PEG-lipid. In some embodiments, the pharmaceutical composition comprises a sterol. In certain embodiments, the pharmaceutical composition comprises a phospholipid.

In some embodiments, the pharmaceutical composition comprises a charged lipid. In certain embodiments, the pharmaceutical composition comprises one or more of a PEG-lipid, sterol, phospholipid, or charged lipid and is formulated as a particle. In some embodiments, the pharmaceutical composition comprises one or more of a PEG-lipid, sterol, phospholipid, or charged lipid and is formulated as a nanoparticle or microparticle. In certain embodiments, the pharmaceutical composition comprises one or more of a PEG-lipid, sterol, phospholipid, or charged lipid and is formulated as a lipid nanoparticle.

In some embodiments, the pharmaceutical composition comprises approximately 40-60% of a compound of Formula (II) by mass. In some embodiments, the pharmaceutical composition comprises approximately 45-55% of a compound of Formula (II) by mass. In some embodiments, the pharmaceutical composition comprises approximately 50% of a compound of Formula (II) by mass.

In some embodiments, the pharmaceutical composition comprises approximately 15-35% of a charged lipid by mass. In some embodiments, the pharmaceutical composition comprises approximately 20-30% of a charged lipid by mass. In some embodiments, the pharmaceutical composition comprises approximately 25% of a charged lipid by mass.

In some embodiments, the pharmaceutical composition comprises approximately 5-25% of a sterol by mass. In some embodiments, the pharmaceutical composition comprises approximately 10-20% of a sterol by mass. In some embodiments, the pharmaceutical composition comprises approximately 15% of a sterol by mass.

In some embodiments, the pharmaceutical composition comprises approximately 0-20% of a PEG-lipid by mass. In some embodiments, the pharmaceutical composition comprises approximately 5-15% of a PEG-lipid by mass. In some embodiments, the pharmaceutical composition comprises approximately 10% of a PEG-lipid by mass.

In some embodiments, the pharmaceutical composition comprises approximately 50% of a compound of Formula (II), approximately 25% of a charged lipid, approximately 15% of a sterol, and approximately 10% of a PEG-lipid by mass. In some embodiments, the pharmaceutical composition comprises approximately 50% of a compound of Formula (II), approximately 25% of a charged lipid, approximately 16.5% of a sterol, and approximately 8.5% of a PEG-lipid by mass.

In some embodiments, the pharmaceutical composition comprises a 10:1 ratio of a compound of Formula (II):agent by mass. In some embodiments, the pharmaceutical composition comprises a 10:1 ratio of a compound of Formula (II):polynucleotide by mass. In some embodiments, the pharmaceutical composition comprises a 10:1 ratio of a compound of Formula (II):mRNA by mass.

The present disclosure also provides inhalable dry powder compositions comprising microparticles, wherein the microparticles comprise a polynucleotide, a lipid nanoparticle, and a saccharide excipient, and wherein at least about 50% of the microparticles in the composition have an aerodynamic size of about 1 micron to about 5 microns, a physical diameter of about 3 microns to about 10 microns, or a combination thereof. In some embodiments, the polynucleotide is encapsulated by the lipid nanoparticle.

In some embodiments, at least about 70% of the microparticles in the composition have an aerodynamic size of about 1 micron to about 5 microns. In certain embodiments, at least about 70% of the microparticles in the composition have a physical diameter of between about 3 microns and about 10 microns.

In certain embodiments, the polynucleotide is any polynucleotide provided herein. In some embodiments, the polynucleotide is at least about 30 nucleotides in length. In certain embodiments, the polynucleotide is at least about 1,000 nucleotides in length. In some embodiments, the polynucleotide is a messenger RNA (mRNA). In certain embodiments, the mRNA encodes an antigen or a therapeutic protein. In some embodiments, the mRNA encodes an antigen. In certain embodiments, the mRNA encodes a therapeutic protein. In some embodiments, the antigen is an antigen from a pathogen or a cancer-associated antigen. In some embodiments, the antigen is an antigen from a pathogen. In some embodiments, the antigen is a cancer-associated antigen.

In certain embodiments, the saccharide excipient comprises a polyol. In some embodiments, the polyol is mannitol or erythritol. In some embodiments, the polyol is mannitol. In certain embodiments, the polyol is erythritol. In some embodiments, the saccharide comprises an oligosaccharide. In some embodiments, the oligosaccharide is trehalose or lactose. In some embodiments, the oligosaccharide is trehalose. In some embodiments, the oligosaccharide is lactose. In some embodiments, the saccharide excipient is mannitol, erythritol, trehalose, or lactose. In some embodiments, the saccharide excipient is mannitol, erythritol, or trehalose. In some embodiments, the saccharide excipient is mannitol. In some embodiments, the saccharide excipient is erythritol. In some embodiments, the saccharide excipient is trehalose. In some embodiments, the saccharide excipient is lactose.

In some embodiments, the lipid nanoparticle comprises an ionizable cationic lipid and one or more of a PEG-lipid, a phospholipid, a charged lipid, and a sterol. In some embodiments, the lipid nanoparticle comprises an ionizable cationic lipid and one or more of a PEG-lipid, a phospholipid, a charged lipid, and cholesterol.

In some embodiments, the ionizable lipid is CKK-E12 (MD-1), C12-200, Dlin-MC3-DMA (MC3), a compound of Formula (I), a compound of Formula (II), or a pharmaceutically acceptable salt, solvate, tautomer, stereoisomer, or isotopically labeled derivative thereof, or a lipid molecule that is neutral at physiological pH, but will be protonated at lower pH, which makes it positively charged. In certain embodiments, the ionizable lipid is CKK-E12 (MD-1), C12-200, Dlin-MC3-DMA (MC3), RCB-01-223-3, RCB-02-76-3, or a pharmaceutically acceptable salt, solvate, tautomer, stereoisomer, or isotopically labeled derivative thereof or a lipid molecule that is neutral at physiological pH, but will be protonated at lower pH, which makes it positively charged. In some embodiments, the ionizable lipid is CKK-E12 (MD-1), C12-200, Dlin-MC3-DMA (MC3), a compound of Formula (I), or a pharmaceutically acceptable salt, solvate, tautomer, stereoisomer, or isotopically labeled derivative thereof, or a lipid molecule that is neutral at physiological pH, but will be protonated at lower pH, which makes it positively charged. In some embodiments, the ionizable lipid is CKK-E12 (MD-1), C12-200, Dlin-MC3-DMA (MC3), a compound of Formula (II), or a pharmaceutically acceptable salt, solvate, tautomer, stereoisomer, or isotopically labeled derivative thereof, or a lipid molecule that is neutral at physiological pH, but will be protonated at lower pH, which makes it positively charged.

In some embodiments, the ionizable lipid is CKK-E12 (MD-1), or a pharmaceutically acceptable salt, solvate, tautomer, stereoisomer, or isotopically labeled derivative thereof. In some embodiments, the ionizable lipid is CKK-E12 (MD-1). In certain embodiments, the ionizable lipid is C12-200, or a pharmaceutically acceptable salt, solvate, tautomer, stereoisomer, or isotopically labeled derivative thereof. In certain embodiments, the ionizable lipid is C12-200. In some embodiments, the ionizable lipid is Dlin-MC3-DMA (MC3), or a pharmaceutically acceptable salt, solvate, tautomer, stereoisomer, or isotopically labeled derivative thereof. In some embodiments, the ionizable lipid is Dlin-MC3-DMA (MC3). In some embodiments, the ionizable lipid is a compound provided herein (e.g., a compound of Formula (I) or (II)), or a pharmaceutically acceptable salt, solvate, tautomer, stereoisomer, or isotopically labeled derivative thereof. In some embodiments, the ionizable lipid is a compound of Formula (I), or a pharmaceutically acceptable salt, solvate, tautomer, stereoisomer, or isotopically labeled derivative thereof. In some embodiments, the ionizable lipid is a compound of Formula (II), or a pharmaceutically acceptable salt, solvate, tautomer, stereoisomer, or isotopically labeled derivative thereof. In some embodiments, the ionizable lipid is RCB-01-223-3 or RCB-02-76-3, or a pharmaceutically acceptable salt, solvate, tautomer, stereoisomer, or isotopically labeled derivative thereof. In some embodiments, the ionizable lipid is RCB-01-223-3, or a pharmaceutically acceptable salt, solvate, tautomer, stereoisomer, or isotopically labeled derivative thereof. In some embodiments, the ionizable lipid is RCB-01-223-3. In some embodiments, the ionizable lipid is RCB-02-76-3, or a pharmaceutically acceptable salt, solvate, tautomer, stereoisomer, or isotopically labeled derivative thereof. In certain embodiments, the ionizable lipid is RCB-02-76-3. In some embodiments, the ionizable lipid is a lipid molecule that is neutral at physiological pH, but will be protonated at lower pH, which makes it positively charged.

In certain embodiments, the charged lipid is any charged lipid provided herein. In some embodiments, the charged lipid is 1,2-dioleoyl-3-trimethylammonium-propane (DOTAP), dimethyldioctadecylammonium (DDA), or a combination thereof. In certain embodiments, the charged lipid is DOTAP. In some embodiments, the charged lipid is DDA. In some embodiments, the charged lipid is a cationic lipid. In some embodiments, the cationic lipid is 1,2-dioleoyl-3-trimethylammonium-propane (DOTAP), dimethyldioctadecylammonium (DDA), or a combination thereof. In certain embodiments, the cationic lipid is DOTAP. In some embodiments, the cationic lipid is DDA.

In certain embodiments, the phospholipid is any phospholipid provided herein. In some embodiments, the phospholipid is 1,2-dioleoyl-sn-glycero-3-phosphoethanolamine (DOPE) or 1,2-distearoyl-sn-glycero-3-phosphocholine (DSPC), or a combination thereof. In certain embodiments, the phospholipid is DOPE. In some embodiments, the phospholipid is DSPC.

In some embodiments, the lipid nanoparticle further comprises a lipid conjugated to a solubilizing group. In certain embodiments, the solubilizing group is a polymer of polyethylene glycol (PEG). In some embodiments, the lipid nanoparticle further comprises any PEG-lipid provided herein.

In some embodiments, the lipid nanoparticle further comprises any sterol provided herein. In certain embodiments, the lipid nanoparticle further comprises a substituted or unsubstituted cholesterol. In certain embodiments, the lipid nanoparticle further comprises a substituted cholesterol. In certain embodiments, the lipid nanoparticle further comprises an unsubstituted cholesterol.

In some embodiments, the composition is a pharmaceutical composition. In some embodiments, the composition is a pharmaceutical composition comprising at least one pharmaceutically acceptable excipient. In some embodiments, the pharmaceutical composition is a vaccine.

In some embodiments, the composition is formulated for endotracheal, mucosal, intranasal, inhalation or pulmonary delivery. In some embodiments, the composition is formulated for endotracheal delivery. In some embodiments, the composition is formulated for mucosal delivery. In some embodiments, the composition is formulated for intranasal delivery. In some embodiments, the composition is formulated for inhalation. In some embodiments, the composition is formulated for pulmonary delivery.

In some embodiments, at least some of the saccharide excipient in the composition is encapsulated by the lipid nanoparticles.

In some embodiments, the composition is spray freeze dried.

Pharmaceutical compositions described herein can be prepared by any method known in the art of pharmaceutics. In general, such preparatory methods include bringing a compound, agent, or particle described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents such as calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor*, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (II) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound, agent, or particle described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound, agent, or particle in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds, agents, or particles provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound, agent, or particle required to achieve an effective amount will vary from subject to subject, depending, for example, on species, age, and general condition of a subject, severity of the side effects or disorder, identity of the particular compound, agent or particle, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound, agent, or particle described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound, agent, or particle described herein. In certain embodiments, a dose described herein includes independently between 1 mg and 3 mg, inclusive, of a compound, agent, or particle described herein. In certain embodiments, a dose described herein includes independently between 3 mg and 10 mg, inclusive, of a compound, agent, or particle described herein. In certain embodiments, a dose described herein includes independently between 10 mg and 30 mg, inclusive, of a compound, agent, or particle described herein. In certain embodiments, a dose described herein includes independently between 30 mg and 100 mg, inclusive, of a compound, agent, or particle described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that treat a disease in a subject in need thereof, prevent a disease in a subject in need thereof, or reduce the risk to develop a disease in a subject in need thereof, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, polynucleotides, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., lung disease or liver disease). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

### PEG-Lipid

In some embodiments, the PEG-lipid is a PEG-phospholipid or PEG-glyceride lipid. In certain embodiments, the PEG-lipid is 1,2-dimyristoyl-sn-glycero-3-phosphoethanolamine-N-[methoxy(polyethylene glycol)-2000](C₁₄PEG2000) or 1,2-dimyristoyl-rac-glycero-3-methoxypolyethylene glycol-2000 (DMG-PEG2000).

In certain embodiments, the PEG-lipid is a PEG-phospholipid. In certain embodiments, the PEG-phospholipid is a PEG-phosphoethanolamine. In some embodiments, the PEG-phospholipid is a PEG-phosphatidylcholine.

In certain embodiments, the PEG component has a molecular weight of about 350, about 550, about 750, about 1000, about 2000, about 3000, or about 5000. In some embodiments, the PEG component has a molecular weight of about 750, about 1000, about 2000, about 3000, or about 5000. In certain embodiments, the PEG component has a molecular weight of about 1000, about 2000, or about 3000. In some embodiments, the PEG component has a molecular weight of about 2000.

In certain embodiments, the PEG-lipid is stearoyl-substituted (C₁₈). In some embodiments, the PEG-phospholipid is palmitoyl-substituted (C₁₆). In certain embodiments, the PEG-phospholipid is myristoyl-substituted (C₁₄).

In certain embodiments, the PEG-lipid is selected from the group consisting of 1,2-distearoyl-sn-glycero-3-phosphoethanolamine-N-[methoxy(polyethylene glycol)-5000](C₁₈PEG5000), 1,2-dipalmitoyl-sn-glycero-3-phosphoethanolamine-N-[methoxy(polyethylene glycol)-5000](C₁₆PEG5000), 1,2-dimyristoyl-sn-glycero-3-phosphoethanolamine-N-[methoxy(polyethylene glycol)-5000](C₁₄PEG5000), 1,2-distearoyl-sn-glycero-3-phosphoethanolamine-N-[methoxy(polyethylene glycol)-3000](C₁₈PEG3000), 1,2-dipalmitoyl-sn-glycero-3-phosphoethanolamine-N-[methoxy(polyethylene glycol)-3000](C₁₆PEG3000), 1,2-dimyristoyl-sn-glycero-3-phosphoethanolamine-N-[methoxy(polyethylene glycol)-3000](C₁₄PEG3000), 1,2-distearoyl-sn-glycero-3-phosphoethanolamine-N-[methoxy(polyethylene glycol)-2000](C₁₈PEG2000), 1,2-dipalmitoyl-sn-glycero-3-phosphoethanolamine-N—[methoxy(polyethylene glycol)-2000](C₁₆PEG2000), 1,2-dimyristoyl-sn-glycero-3-phosphoethanolamine-N-[methoxy(polyethylene glycol)-2000](C₁₄PEG2000), 1,2-distearoyl-sn-glycero-3-phosphoethanolamine-N-[methoxy(polyethylene glycol)-1000](C₁₈PEGI000), 1,2-dipalmitoyl-sn-glycero-3-phosphoethanolamine-N-[methoxy(polyethylene glycol)-1000](C₁₆PEG1000), 1,2-dimyristoyl-sn-glycero-3-phosphoethanolamine-N-[methoxy(polyethylene glycol)-1000](C₁₄PEG1000), 1,2-distearoyl-sn-glycero-3-phosphoethanolamine-N-[methoxy(polyethylene glycol)-750](C₁₈PEG750), 1,2-dipalmitoyl-sn-glycero-3-phosphoethanolamine-N-[methoxy(polyethylene glycol)-750](C₁₆PEG750), and 1,2-dimyristoyl-sn-glycero-3-phosphoethanolamine-N-[methoxy(polyethylene glycol)-750](C₁₄PEG750). In some embodiments, the PEG-lipid is selected from the group consisting of 1,2-distearoyl-sn-glycero-3-phosphoethanolamine-N-[methoxy(polyethylene glycol)-2000](C₁₈PEG2000), 1,2-dipalmitoyl-sn-glycero-3-phosphoethanolamine-N-[methoxy(polyethylene glycol)-2000](C₁₆PEG2000), and 1,2-dimyristoyl-sn-glycero-3-phosphoethanolamine-N-[methoxy(polyethylene glycol)-2000](C₁₄PEG2000). In certain embodiments, the PEG-lipid is selected from the group consisting of 1,2-dimyristoyl-sn-glycero-3-phosphoethanolamine-N-[methoxy(polyethylene glycol)-5000](C₁₄PEG5000), 1,2-dimyristoyl-sn-glycero-3-phosphoethanolamine-N-[methoxy(polyethylene glycol)-3000](C₁₄PEG3000), 1,2-dimyristoyl-sn-glycero-3-phosphoethanolamine-N-[methoxy(polyethylene glycol)-2000](C₁₄PEG2000), 1,2-dimyristoyl-sn-glycero-3-phosphoethanolamine-N-[methoxy(polyethylene glycol)-1000](C₁₄PEG1000), and 1,2-dimyristoyl-sn-glycero-3-phosphoethanolamine-N-[methoxy(polyethylene glycol)-750](C₁₄PEG750). In certain embodiments, the PEG-phospholipid is 1,2-dimyristoyl-sn-glycero-3-phosphoethanolamine-N-[methoxy(polyethylene glycol)-2000](C₁₄PEG2000).

In some embodiments, the PEG-lipid is a PEG-glyceride lipid. In certain embodiments, the PEG-lipid is 1,2-distearoyl-rac-glycero-3-methoxypolyethylene glycol-2000 (DSG-PEG2000) or 1,2-dimyristoyl-rac-glycero-3-methoxypolyethylene glycol-2000 (DMG-PEG2000). In some embodiments, the PEG-lipid is 1,2-distearoyl-rac-glycero-3-methoxypolyethylene glycol-2000 (DSG-PEG2000). In certain embodiments, the PEG-lipid is 1,2-dimyristoyl-rac-glycero-3-methoxypolyethylene glycol-2000 (DMG-PEG2000).

### Sterol

In certain embodiments, the sterol is cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, or a derivative thereof. In some embodiments, the sterol is cholesterol, or a derivative thereof. In certain embodiments, the sterol is cholesterol.

### Charged Lipid

In some embodiments, the charged lipid is a fixed cationic lipid or salt thereof. In some embodiments, the fixed cationic lipid is 1,2-dioleoyl-3-trimethylammonium propane (DOTAP), 1,2-di-O-octadecenyl-3-trimethylammonium propane (DOTMA), 1,2-stearoyl-3-trimethylammonium-propane (18:0 TAP), 1,2-dipalmitoyl-3-trimethylammonium-propane (16:0 TAP), 1,2-dimyristoyl-3-trimethylammonium-propane (14:0 TAP), dimethyldioctadecylammonium (18:0 DDAB), 1,2-dimyristoleoyl-sn-glycero-3-ethylphosphocholine (14:1 EPC), 1-palmitoyl-2-oleoyl-sn-glycero-3-ethylphosphocholine (16:0-18:1 EPC), 1,2-dioleoyl-sn-glycero-3-ethylphosphocholine (18:1 EPC), 1,2-distearoyl-sn-glycero-3-ethylphosphocholine (18:0 EPC), 1,2-dipalmitoyl-sn-glycero-3-ethylphosphocholine (16:0 EPC), 1,2-dimyristoyl-sn-glycero-3-ethylphosphocholine (14:0 EPC), 1,2-dilauroyl-sn-glycero-3-ethylphosphocholine (12:0 EPC), O,O′-ditetradecanoyl-N—(α-trimethylammonioacetyl)diethanolamine (DC-6-14), or N-(2-hydroxyethyl)-N,N-dimethyl-2,3-bis(oleoyloxy)propan-1-aminium. In some embodiments, the fixed cationic lipid is 1,2-dioleoyl-3-trimethylammonium propane (DOTAP).

In some embodiments, the charged lipid is a salt of a fixed cationic ligand. In certain embodiments, the salt of a fixed cationic lipid is a chloride salt, bromide salt, methyl sulfate salt, or triflate salt. In some embodiments, the salt of a fixed cationic ligand is a chloride salt.

In some embodiments, the charged lipid is an ionizable lipid. In certain embodiments, the ionizable lipid is 1,2-distearoyl-3-dimethylammonium-propane (18:0 DAP), 1,2-dipalmitoyl-3-dimethylammonium-propane (16:0 DAP), 1,2-dimyristoyl-3-dimethylammonium-propane (14:0 DAP), 1,2-dioleoyl-3-dimethylammonium-propane (DODAP or 18:1 DAP), or 1,2-dioleyloxy-3-dimethylaminopropane (DODMA).

In some embodiments, the charged lipid is a phospholipid. In certain embodiments, the charged lipid is not a phospholipid.

### Phospholipid

In certain embodiments, the phospholipid is a phosphoethanolamine or phosphatidylcholine. In some embodiments, the phospholipid is 1,2-distearoyl-sn-glycero-3-phosphorylethanolamine (DSPE), 1,2-distearoyl-sn-glycero-3-phosphocholine (DSPC), or 1,2-dioleoyl-sn-glycero-3-phosphoethanolamine (DOPE). In some embodiments, the phospholipid is a phosphoethanolamine. In certain embodiments, the phospholipid is 1,2-distearoyl-sn-glycero-3-phosphorylethanolamine (DSPE) or phospholipid is 1,2-dioleoyl-sn-glycero-3-phosphoethanolamine (DOPE). In certain embodiments, the phospholipid is 1,2-distearoyl-sn-glycero-3-phosphorylethanolamine (DSPE). In certain embodiments, the phospholipid is 1,2-dioleoyl-sn-glycero-3-phosphoethanolamine (DOPE). In some embodiments, the phospholipid is a phosphatidylcholine. In some embodiments, the phospholipid is 1,2-distearoyl-sn-glycero-3-phosphocholine (DSPC).

### Agents

In certain embodiments, the pharmaceutical composition further comprises an agent. In some embodiments, the agent is an organic molecule, inorganic molecule, protein, peptide, polynucleotide, targeting agent, an isotopically labeled chemical compound, vaccine, an immunological agent, or an agent useful in bioprocessing.

In some embodiments, the agent and the compound, or the pharmaceutically acceptable salt, solvate, tautomer, stereoisomer, or isotopically labeled derivative thereof, are not covalently attached.

Agents that are delivered by the systems (e.g., pharmaceutical compositions) described herein may be (e.g., therapeutic or prophylactic), diagnostic, cosmetic, or nutraceutical agents. Any chemical compound to be administered to a subject may be delivered using the complexes, picoparticles, nanoparticles (e.g., lipid nanoparticles), microparticles, micelles, or liposomes, described herein. In some embodiments, the agent is an organic molecule, inorganic molecule, protein, peptide, polynucleotide, targeting agent, an isotopically labeled chemical compound, vaccine, an immunological agent, or an agent useful in bioprocessing (e.g., intracellular manufacturing of proteins, such as a cell's bioprocessing of a commercially useful chemical or fuel). For example, intracellular delivery of an agent may be useful in bioprocessing by maintaining the cell's health and/or growth, e.g., in the manufacturing of proteins. Any chemical compound to be administered to a subject or contacted with a cell may be delivered to the subject or cell using the compositions.

Exemplary agents that may be included in a composition described herein include, but are not limited to, small molecules, organometallic compounds, polynucleotides, proteins, peptides, carbohydrates, monosaccharides, oligosaccharides, polysaccharides, nucleoproteins, mucoproteins, lipoproteins, small molecules linked to proteins, glycoproteins, steroids, nucleotides, oligonucleotides, polynucleotides, nucleosides, antisense oligonucleotides, lipids, hormones, vitamins, cells, metals, targeting agents, isotopically labeled chemical compounds, drugs (e.g., compounds approved for human or veterinary use by the U.S. Food and Drug Administration as provided in the Code of Federal Regulations), vaccines, immunological agents, agents useful in bioprocessing, and mixtures thereof. The targeting agents are described in more detail herein. In certain embodiments, the agents are nutraceutical agents. In certain embodiments, the agents are pharmaceutical agents (e.g., a therapeutic or prophylactic agent). In certain embodiments, the agent is an antibiotic agent (e.g., an anti-bacterial, anti-viral, or anti-fungal agent), anesthetic, steroidal agent, anti-proliferative agent, anti-inflammatory agent, anti-angiogenesis agent, anti-neoplastic agent, anti-cancer agent, anti-diabetic agent, antigen, vaccine, antibody, decongestant, antihypertensive, sedative, birth control agent, progestational agent, anti-cholinergic, analgesic, immunosuppressant, anti-depressant, anti-psychotic, P-adrenergic blocking agent, diuretic, cardiovascular active agent, vasoactive agent, non-steroidal, nutritional agent, anti-allergic agent, or pain-relieving agent. Vaccines may comprise isolated proteins or peptides, inactivated organisms and viruses, dead organisms and viruses, genetically altered organisms or viruses, polynucleotide (e.g., mRNA), and cell extracts. Therapeutic and prophylactic agents may be combined with interleukins, interferon, cytokines, and adjuvants such as cholera toxin, alum, and Freund's adjuvant, etc.

In certain embodiments, an agent to be delivered or used in a composition described herein is a polynucleotide. In certain embodiments, the agent is plasmid DNA (pDNA). In certain embodiments, the agent is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA. In certain embodiments, the agent is RNA. In certain embodiments, the agent is small interfering RNA (siRNA). In certain embodiments, the agent is messenger RNA (mRNA). In certain embodiments, the agent is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or incRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In certain embodiments, the polynucleotide may be provided as an antisense agent or RNAi. See, e.g., Fire et al., Nature 391:806-811, 1998. Antisense therapy is meant to include, e.g., administration or in situ provision of single- or double-stranded polynucleotides, or derivatives thereof, which specifically hybridize, e.g., bind, under cellular conditions, with cellular mRNA and/or genomic DNA, or mutants thereof, so as to inhibit the expression of the encoded protein, e.g., by inhibiting transcription and/or translation. See, e.g., Crooke, “Molecular mechanisms of action of antisense drugs,” Biochim. Biophys. Acta 1489(1):31-44, 1999; Crooke, “Evaluating the mechanism of action of anti-proliferative antisense drugs,” Antisense Nucleic AcidDrugDev. 10(2):123-126, discussion 127, 2000; Methods in Enzymology volumes 313-314, 1999. The binding may be by conventional base pair complementarity, or, for example, in the case of binding to DNA duplexes, through specific interactions in the major groove of the double helix (i.e., triple helix formation). See, e.g., Chan et al., J. Mol. Med. 75(4):267-282, 1997.

In some embodiments, pDNA, siRNA, dsRNA, shRNA, miRNA, mRNA, tRNA, asRNA, and/or RNAi can be designed and/or predicted using one or more of a large number of available algorithms. To give but a few examples, the following resources can be utilized to design and/or predict polynucleotides: algorithms found at Alnylum Online; Dharmacon Online; OligoEngine Online; Molecula Online; Ambion Online; BioPredsi Online; RNAi Web Online; Chang Bioscience Online; Invitrogen Online; LentiWeb Online GenScript Online; Protocol Online; Reynolds et al., 2004, Nat. Biotechnol., 22:326; Naito et al., 2006, Nucleic Acids Res., 34:W448; Li et al., 2007, RNA, 13:1765; Yiu et al., 2005, Bioinformatics, 21:144; and Jia et al., 2006, BMC Bioinformatics, 7: 271.

The polynucleotide included in a composition may be of any size or sequence, and they may be single- or double-stranded. In certain embodiments, the polynucleotide includes at least about 30, at least about 100, at least about 300, at least about 1,000, at least about 3,000, or at least about 10,000 base pairs. In certain embodiments, the polynucleotide includes less than about 10,000, less than about 3,000, less than about 1,000, less than about 300, less than about 100, or less than about 30 base pairs. Combinations of the above ranges (e.g., at least about 100 and less than about 1,000) are also within the scope of the invention. The polynucleotide may be provided by any means known in the art. In certain embodiments, the polynucleotide is engineered using recombinant techniques. See, e.g., Ausubel et al., Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989). The polynucleotide may also be obtained from natural sources and purified from contaminating components found normally in nature. The polynucleotide may also be chemically synthesized in a laboratory. In certain embodiments, the polynucleotide is synthesized using standard solid phase chemistry. The polynucleotide may be isolated and/or purified. In certain embodiments, the polynucleotide is substantially free of impurities. In certain embodiments, the polynucleotide is at least about 50%, at least about 60%, at least about 70%, at least about 80%, at least about 90%, at least about 95%, or at least about 99% free of impurities.

The polynucleotide may be modified by physical, chemical, and/or biological means. The modifications include methylation, phosphorylation, and end-capping, etc. In certain embodiments, the modifications lead to increased stability of the polynucleotide.

Wherever a polynucleotide is employed in the composition, a derivative of the polynucleotide may also be used. These derivatives include products resulted from modifications of the polynucleotide in the base moieties, sugar moieties, and/or phosphate moieties of the polynucleotide. Modified base moieties include, but are not limited to, 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, 0(6)-methylguanine, and 2-thiocytidine. Modified sugar moieties include, but are not limited to, 2′-fluororibose, ribose, 2′-deoxyribose, 3′-azido-2′,3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′—N-phosphoramidite linkages. Combinations of the various modifications may be used in a single polynucleotide. These modified polynucleotides may be provided by any means known in the art; however, as will be appreciated by those of skill in the art, the modified polynucleotides may be prepared using synthetic chemistry in vitro.

The polynucleotide described herein may be in any form, such as a circular plasmid, a linearized plasmid, a cosmid, a viral genome, a modified viral genome, and an artificial chromosome.

The polynucleotide described herein may be of any sequence. In certain embodiments, the polynucleotide encodes a protein or peptide. The encoded protein may be an enzyme, structural protein, receptor, soluble receptor, ion channel, active (e.g., pharmaceutically active) protein, cytokine, interleukin, antibody, antibody fragment, antigen, coagulation factor, albumin, growth factor, hormone, and insulin, etc. The polynucleotide may also comprise regulatory regions to control the expression of a gene. These regulatory regions may include, but are not limited to, promoters, enhancer elements, repressor elements, TATA boxes, ribosomal binding sites, and stop sites for transcription, etc. In certain embodiments, the polynucleotide is not intended to encode a protein. For example, the polynucleotide may be used to fix an error in the genome of the cell being transfected.

In certain embodiments, the polynucleotide described herein comprises a sequence encoding an antigenic peptide or protein. A composition containing the polynucleotide can be delivered to a subject to induce an immunologic response sufficient to decrease the chance of a subsequent infection and/or lessen the symptoms associated with such an infection. The polynucleotide of these vaccines may be combined with interleukins, interferon, cytokines, and/or adjuvants described herein.

The antigenic protein or peptides encoded by the polynucleotide may be derived from bacterial organisms, such as Streptococccus pneumoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, and Camphylobacter jejuni; from viruses, such as smallpox virus, influenza A virus, influenza B virus, respiratory syncytial virus, parainfluenza virus, measles virus, HIV virus, varicella-zoster virus, herpes simplex 1 virus, herpes simplex 2 virus, cytomegalovirus, Epstein-Barr virus, rotavirus, rhinovirus, adenovirus, papillomavirus, poliovirus, mumps virus, rabies virus, rubella virus, coxsackieviruses, equine encephalitis virus, Japanese encephalitis virus, yellow fever virus, Rift Valley fever virus, hepatitis A virus, hepatitis B virus, hepatitis C virus, hepatitis D virus, and hepatitis E virus; and from fungal, protozoan, or parasitic organisms, such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, and Schistosoma mansoni.

In certain embodiments, the agent is erythropoietin (EPO), e.g., recombinant human erythropoietin (rhEPO). Erythropoietin is an essential hormone for red blood cell production, and may be used in treating hematological diseases, e.g., anemia., such as anemia resulting from chronic kidney disease, chemotherapy induced anemia in patients with cancer, inflammatory bowel disease (Crohn's disease and ulcerative colitis) and myelodysplasia from the treatment of cancer (chemotherapy and radiation). Recombinant human erythropoietins available for use include EPOGEN/PROCRIT (Epoetin alfa, rINN) and ARANESP (Darbepoetin alfa, rINN).

An agent described herein may be non-covalently (e.g., complexed or encapsulated) attached to a compound as described herein, or included in a composition described herein. In certain embodiments, upon delivery of the agent into a cell, the agent is able to interfere with the expression of a specific gene in the cell.

In certain embodiments, the agent in a composition that is delivered to a subject in need thereof may be a mixture of two or more agents that may be useful as, e.g., combination therapies. The compositions including the two or more agents can be administered to achieve a synergistic effect. In certain embodiments, the compositions including the two or more agents can be administered to improve the activity and/or bioavailability, reduce and/or modify the metabolism, inhibit the excretion, and/or modify the distribution within the body of a subject, of each one of the two or more agents. It will also be appreciated that the therapy employed may achieve a desired effect for the same disorder, and/or it may achieve different effects.

The compositions (e.g., pharmaceutical compositions) can be administered concurrently with, prior to, or subsequent to the one or more agents (e.g., pharmaceutical agents). The two or more agents may be useful for treating and/or preventing a same disease or different diseases described herein. Each one of the agents may be administered at a dose and/or on a time schedule determined for that agent. The agents may also be administered together with each other and/or with the composition described herein in a single dose or administered separately in different doses. The particular combination to employ in a regimen will take into account compatibility of the agents and/or the desired therapeutic and/or prophylactic effect to be achieved. In general, it is expected that the agents utilized in combination be utilized at levels that do not exceed the levels at which they are utilized individually. In some embodiments, the levels utilized in combination will be lower than those utilized individually.

### Targeting Agents

Since it is often desirable to target a particular cell, collection of cells, or tissue, compounds provided herein, and the complexes, liposomes, micelles, and particles (e.g., microparticles and nanoparticles) thereof, may be modified to include targeting moieties.

For example, a compound provided herein may include a targeting moiety. A variety of agents or regions that target particular cells are known in the art. See, e.g., Cotten et al., Methods Enzym. 217:618, 1993. The targeting agent may be included throughout a particle of a compound provided herein or may be only on the surface of the particle. The targeting agent may be a protein, peptide, carbohydrate, glycoprotein, lipid, small molecule, or polynucleotide, etc. The targeting agent may be used to target specific cells or tissues or may be used to promote endocytosis or phagocytosis of the particle. Examples of targeting agents include, but are not limited to, antibodies, fragments of antibodies, proteins, peptides, carbohydrates, receptor ligands, sialic acid, and aptamers, etc. If the targeting agent is included throughout a particle, the targeting agent may be included in the mixture that is used to form the particle. If the targeting agent is only on the surface of a particle, the targeting agent may be associated with (e.g., by covalent or non-covalent (e.g., electrostatic, hydrophobic, hydrogen bonding, van der Waals, π-π stacking) interactions) the formed particle using standard chemical techniques.

### Particles

In some embodiments, a composition including a compound provided herein and an agent is in the form of a particle. In certain embodiments, the compound provided herein and agent form a complex, and the complex is in the form of a particle. In certain embodiments, the compound provided herein encapsulates the agent and is in the form of a particle. In certain embodiments, the compound provided herein is mixed with the agent, and the mixture is in the form of a particle. In some embodiments, the particle encapsulates the agent.

In certain embodiments, a complex of a compound provided herein and an agent in a composition of is in the form of a particle. In some embodiments, the particle is a nanoparticle or a microparticle. In certain embodiments, the particle is a microparticle (i.e., particle having a characteristic dimension of less than about 1 millimeter and at least about 1 micrometer, where the characteristic dimension of the particle is the smallest cross-sectional dimension of the particle). In certain embodiments, the particle is a nanoparticle (i.e., a particle having a characteristic dimension of less than about 1 micrometer and at least about 1 nanometer, where the characteristic dimension of the particle is the smallest cross-sectional dimension of the particle). In certain embodiments, the average diameter of the particle is at least about 10 nm, at least about 30 nm, at least about 100 nm, at least about 300 nm, at least about 1 μm, at least about 3 μm, at least about 10 μm, at least about 30 μm, at least about 100 μm, at least about 300 μm, or at least about 1 mm. In certain embodiments, the average diameter of the particle is less than about 1 mm, less than about 300 μm, less than about 100 μm, less than about 30 μm less than about 10 μm, less than about 3 μm, less than about 1 μm, less than about 300 nm, less than about 100 nm, less than about 30 nm, or less than about 10 nm. Combinations of the above ranges (e.g., at least about 100 nm and less than about 1 μm) are also within the scope of the present invention.

The particles described herein may include additional materials such as polymers (e.g., synthetic polymers (e.g., PEG, PLGA) and natural polymers (e.g., phospholipids)). In certain embodiments, the additional materials are approved by a regulatory agency, such as the U.S. FDA, for human and veterinary use.

The particles may be prepared using any method known in the art, such as precipitation, milling, spray drying, single and double emulsion solvent evaporation, solvent extraction, phase separation, and simple and complex coacervation. In certain embodiments, methods of preparing the particles are the double emulsion process and spray drying. The conditions used in preparing the particles may be altered to yield particles of a desired size or property (e.g., hydrophobicity, hydrophilicity, external morphology, “stickiness”, shape, polydispersity, etc.). The method of preparing the particle and the conditions (e.g., solvent, temperature, concentration, and air flow rate, etc.) used may also depend on the agent being complexed, encapsulated, or mixed, and/or the composition of the matrix.

Methods developed for making particles for delivery of agents that are included in the particles are described in the literature. See, e.g., Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al., Reactive Polymers 6:275-283, 1987; Mathiowitz et al., J. Appl. Polymer Sci. 35:755-774, 1988.

If the particles prepared by any of the above methods have a size range outside of the desired range, the particles can be sized, for example, using a sieve. The particles may also be coated. In certain embodiments, the particles are coated with a targeting agent. In certain embodiments, the particles are coated with a surface-altering agent. In some embodiments, the particles are coated to achieve desirable surface properties (e.g., a particular charge).

In certain embodiments, the polydispersity index (PDI, determined by dynamic light scattering) of the particles described herein (e.g., particles included in a composition described herein) is between 0.01 and 0.9, between 0.1 and 0.9, between 0.1 and 0.7, between 0.1 and 0.5, between 0.01 and 0.4, between 0.03 and 0.4, between 0.1 and 0.4, between 0.01 and 0.3, between 0.03 and 0.3, or between 0.1 and 0.3.

### Micelles and Liposomes

A composition including one or more compounds provided herein and an agent may be in the form of a micelle, liposome, or lipoplex. In certain embodiments, the compound provided herein is in the form of a micelle or liposome. In certain embodiments, the agent is in the form of a micelle or liposome. In certain embodiments, the compound provided herein and agent form a complex, and the complex is in the form of a micelle or liposome. In certain embodiments, the compound provided herein encapsulates the agent and is in the form of a micelle or liposome. In certain embodiments, the compound provided herein is mixed with the agent, and the mixture is in the form of a micelle or liposome. Micelles and liposomes are particularly useful in delivering an agent, such as a hydrophobic agent. When the micelle or liposome is complexed with (e.g., encapsulates or covers) a polynucleotide, the resulting complex may be referred to as a “lipoplex.” Many techniques for preparing micelles and liposomes are known in the art, and any such method may be used herein to make micelles and liposomes.

In certain embodiments, liposomes are formed through spontaneous assembly. In some embodiments, liposomes are formed when thin lipid films or lipid cakes are hydrated and stacks of lipid crystalline bilayers become fluid and swell. The hydrated lipid sheets detach during agitation and self-close to form large, multilamellar vesicles (LMV). This prevents interaction of water with the hydrocarbon core of the bilayers at the edges. Once these liposomes have formed, reducing the size of the liposomes can be modified through input of sonic energy (sonication) or mechanical energy (extrusion). See, e.g., Walde, P. “Preparation of Vesicles (Liposomes)” In Encylopedia of Nanoscience and Nanotechnology; Nalwa, H. S. Ed. American Scientific Publishers: Los Angeles, 2004; Vol. 9, pp. 43-79; Szoka et al., “Comparative Properties and Methods of Preparation of Lipid Vesicles (Liposomes)” Ann. Rev. Biophys. Bioeng. 9:467-508, 1980; each of which is incorporated herein by reference. The preparation of lipsomes may involve preparing a compound provided herein for hydration, hydrating the compound with agitation, and sizing the vesicles to achieve a homogenous distribution of liposomes. A compound provided herein may be first dissolved in an organic solvent in a container to result in a homogeneous mixture. The organic solvent is then removed to form a polymer-derived film. This polymer-derived film is thoroughly dried to remove residual organic solvent by placing the container on a vacuum pump for a period of time. Hydration of the polymer-derived film is accomplished by adding an aqueous medium and agitating the mixture. Disruption of LMV suspensions using sonic energy typically produces small unilamellar vesicles (SUV) with diameters in the range of 15-50 nm. Lipid extrusion is a technique in which a lipid/polymer suspension is forced through a polycarbonate filter with a defined pore size to yield particles having a diameter near the pore size of the filter used. Extrusion through filters with 100 nm pores typically yields large, unilamellar polymer-derived vesicles (LUV) with a mean diameter of 120-140 nm. In certain embodiments, the amount of a compound provided herein in the liposome ranges from about 30 mol % to about 80 mol %, from about 40 mol % to about 70 mol %, or from about 60 mol % to about 70 mol %. In certain embodiments, the compound provided herein employed further complexes an agent, such as a polynucleotide. In such embodiments, the application of the liposome is the delivery of the polynucleotide.

The following scientific papers described other methods for preparing liposomes and micelles: Narang et al., “Cationic Lipids with Increased DNA Binding Affinity for Nonviral Gene Transfer in Dividing and Nondividing Cells,” Bioconjugate Chem. 16:156-68, 2005; Hofland et al., “Formation of stable cationic lipid/DNA complexes for gene transfer,” Proc. Natl. Acad. Sci. USA 93:7305-7309, July 1996; Byk et al., “Synthesis, Activity, and Structure—Activity Relationship Studies of Novel Cationic Lipids for DNA Transfer,” J. Med. Chem. 41(2):224-235, 1998; Wu et al., “Cationic Lipid Polymerization as a Novel Approach for Constructing New DNA Delivery Agents,” Bioconjugate Chem. 12:251-57, 2001; Lukyanov et al., “Micelles from lipid derivatives of water-soluble polymers as delivery systems for poorly soluble drugs,” Advanced Drug Delivery Reviews 56:1273-1289, 2004; Tranchant et al., “Physicochemical optimisation of plasmid delivery by cationic lipids,” J. Gene Med. 6:S24-S35, 2004; van Balen et al., “Liposome/Water Lipophilicity: Methods, Information Content, and Pharmaceutical Applications,” Medicinal Research Rev. 24(3):299-324, 2004.

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for preventing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for reducing the risk of developing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits are useful for delivering an agent to a subject or cell. In certain embodiments, the kits are useful for delivering a polynucleotide to a subject or cell. In certain embodiments, the kits are useful for delivering mRNA to a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for preventing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for reducing the risk of developing a disease (e.g., genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease) in a subject in need thereof. In certain embodiments, the kits and instructions provide for delivering an agent to a subject or cell. In certain embodiments, the kits and instructions provide for delivering a polynucleotide to a subject or cell. In certain embodiments, the kits and instructions provide for delivering mRNA to a subject or cell. A kit described herein may include one or more additional pharmaceutical agents described herein as a separate composition.

### Methods of Treatment and Uses

Also provided herein are methods for treating and/or preventing a disease, disorder, or condition in a subject, comprising administering to the subject a composition comprising an agent and a compound provided herein (e.g., a compound of Formula (I) or Formula (II)), or a pharmaceutically acceptable salt, solvate, tautomer, stereoisomer, or isotopically labeled derivative thereof.

Also provided herein are methods of treating or preventing a disorder or a disease by administering to a subject in need thereof a therapeutically effective amount of an inhalable dry powder composition provided herein.

Also provided herein are uses of an inhalable dry powder composition provided herein for the prophylaxis, treatment, and/or amelioration of a disorder or a disease.

In certain embodiments, the disease, disorder, or condition is a genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease. In some embodiments, the disease, disorder, or condition is a genetic disease. In some embodiments, the disease, disorder, or condition is a proliferative disease. In some embodiments, the disease, disorder, or condition is a hematological disease. In some embodiments, the disease, disorder, or condition is a neurological disease. In some embodiments, the disease, disorder, or condition is a liver disease. In some embodiments, the disease, disorder, or condition is a spleen disease. In some embodiments, the disease, disorder, or condition is a lung disease. In some embodiments, the disease, disorder, or condition is a painful condition. In some embodiments, the disease, disorder, or condition is a psychiatric disorder. In some embodiments, the disease, disorder, or condition is a musculoskeletal disease. In some embodiments, the disease, disorder, or condition is a metabolic disorder. In some embodiments, the disease, disorder, or condition is an inflammatory disease. In some embodiments, the disease, disorder, or condition is an autoimmune disease.

In some embodiments, the disorder or disease is an allergic disease, an autoimmune disease, an infectious disease, or a cancer. In certain embodiments, the disorder or disease is an allergic disease. In some embodiments, the disorder or disease is an autoimmune disease.

In certain embodiments, the disorder or disease is an infectious disease. In some embodiments, the infectious disease is a viral, bacterial, or protozoological infectious disease.

In some embodiments, the infectious disease is a viral disease. In some embodiments, the infectious disease is a bacterial disease. In some embodiments, the infectious disease is a protozoological infectious disease. In some embodiments, the disorder or disease is a cancer.

In some embodiments, the disorder or disease is a lung disease, a cardiovascular disease, or a neuronal disease. In some embodiments, the disorder or disease is a lung disease. In some embodiments, the lung disease is asthma, chronic obstructive pulmonary disease (COPD), cystic fibrosis (CF), lymphangioleiomyomatosis (LAM) or pulmonary fibrosis. In some embodiments, the lung disease is asthma. In some embodiments, the lung disease is chronic obstructive pulmonary disease (COPD). In some embodiments, the lung disease is cystic fibrosis (CF). In some embodiments, the lung disease is lymphangioleiomyomatosis (LAM). In some embodiments, the lung disease is pulmonary fibrosis. In some embodiments, the disorder or disease is a cardiovascular disease. In some embodiments, the disorder or disease is a neuronal disease.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal.

In certain embodiments, the animal is a transgenic animal (e.g., transgenic mice and transgenic pigs). In certain embodiments, the subject is a fish or reptile.

In some embodiments, the agent is any agent provided herein. In certain embodiments, the agent is a polynucleotide. In some embodiments, the agent is mRNA.

### Additional Methods and Uses

Also provided herein are methods of delivering an agent (e.g., a polynucleotide), comprising administering a composition comprising a polynucleotide and a compound provided herein (e.g., a compound of Formula (I) or (II)), or a pharmaceutically acceptable salt, solvate, tautomer, stereoisomer, or isotopically labeled derivative thereof. In certain embodiments, the method is for delivering an agent to a subject, cell, collection of cells, or tissue. In some embodiments, the method is for delivering an agent to a subject or cell. In certain embodiments, the method is for delivering an agent to a subject. In some embodiments, the method is for delivering an agent to a cell.

In some embodiments, the agent is any agent provided herein. In certain embodiments, the agent is a polynucleotide. In some embodiments, the agent is mRNA.

In some embodiments, the agent is delivered to a subject. In some embodiments, the agent is delivered to the lungs, liver, or spleen of the subject. In certain embodiments, the agent is delivered to the lungs of the subject. In some embodiments, the agent is delivered to the liver of the subject. In certain embodiments, the agent is delivered to the spleen of the subject. In some embodiments, the polynucleotide is delivered to a subject. In some embodiments, the polynucleotide is delivered to the lungs, liver, or spleen of the subject. In certain embodiments, the polynucleotide is delivered to the lungs of the subject. In some embodiments, the polynucleotide is delivered to the liver of the subject. In certain embodiments, the polynucleotide is delivered to the spleen of the subject. In some embodiments, the mRNA is delivered to a subject. In some embodiments, the mRNA is delivered to the lungs, liver, or spleen of the subject. In certain embodiments, the mRNA is delivered to the lungs of the subject. In some embodiments, the mRNA is delivered to the liver of the subject. In certain embodiments, the mRNA is delivered to the spleen of the subject.

In certain embodiments, the agent is delivered to a cell. In certain embodiments, the polynucleotide is delivered to a cell. In certain embodiments, the mRNA is delivered to a cell. In some embodiments, the cell is in vivo, e.g., in an organism. In certain embodiments, the cell is in vitro, e.g., in cell culture. In some embodiments, the cell is ex vivo, meaning the cell is removed from an organism prior to the delivery.

In some embodiments, the composition is administered by any method provided herein. In certain embodiments, the composition is administered by inhalation, intravenously, intratracheally, or intramuscularly. In some embodiments, the composition is administered by inhalation. In certain embodiments, the composition is administered intravenously. In some embodiments, the composition is administered intratracheally. In certain embodiments, the composition is administered intramuscularly.

In another aspect, the disclosure provides a method of preparing a compound of Formula (I), the method comprising reacting a compound of Formula (III):

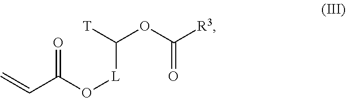

or a salt thereof, with a compound of formula:

H—X,

or a salt thereof, wherein:
T is optionally substituted C₁-C₂₀ alkyl, optionally substituted C₂-C₂₀ alkenyl, or optionally substituted C₂-C₂₀ alkynyl;
L is optionally substituted C₁-C₂₀ alkylene, optionally substituted C₂-C₂₀ alkenylene, or optionally substituted C₂-C₂₀ alkynylene;
R³ is optionally substituted aliphatic or optionally substituted heteroaliphatic;
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group; and
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl.

Also provided herein is a method of preparing a compound of Formula (II), the method comprising reacting a compound of Formula (IV):

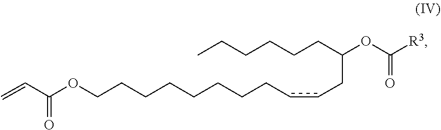

or a salt thereof, with a compound of formula:

H—X,

or a salt thereof, wherein:
R³ is optionally substituted aliphatic or optionally substituted heteroaliphatic;
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl; and

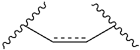

represents either a single or a double bond.

As defined herein, T is optionally substituted C₁-C₂₀ alkyl, optionally substituted C₂-C₂₀ alkenyl, or optionally substituted C₂-C₂₀ alkynyl. In some embodiments, T is unsubstituted C₁-C₂₀ alkyl, unsubstituted C₂-C₂₀ alkenyl, or unsubstituted C₂-C₂₀ alkynyl. In some embodiments, T is unsubstituted C₁-C₂₀ alkyl or unsubstituted C₂-C₂₀ alkenyl. In some embodiments, T is unsubstituted C₁-C₂₀ alkyl. In some embodiments, T is unsubstituted C₁-C₁₀ alkyl. In some embodiments, T is unsubstituted C₅-C₁₀ alkyl. In some embodiments, T is unsubstituted C₁-C₂₀ alkenyl. In some embodiments, T is unsubstituted C₁-C₁₀ alkenyl. In some embodiments, T is unsubstituted C₅-C₁₀ alkenyl. In some embodiments, T is unsubstituted C₁-C₂₀ alkenyl with one double bond. In some embodiments, T is unsubstituted C₁-C₁₀ alkenyl with one double bond. In some embodiments, T is unsubstituted C₅-C₁₀ alkenyl with one double bond.

In some embodiments, T is selected from:

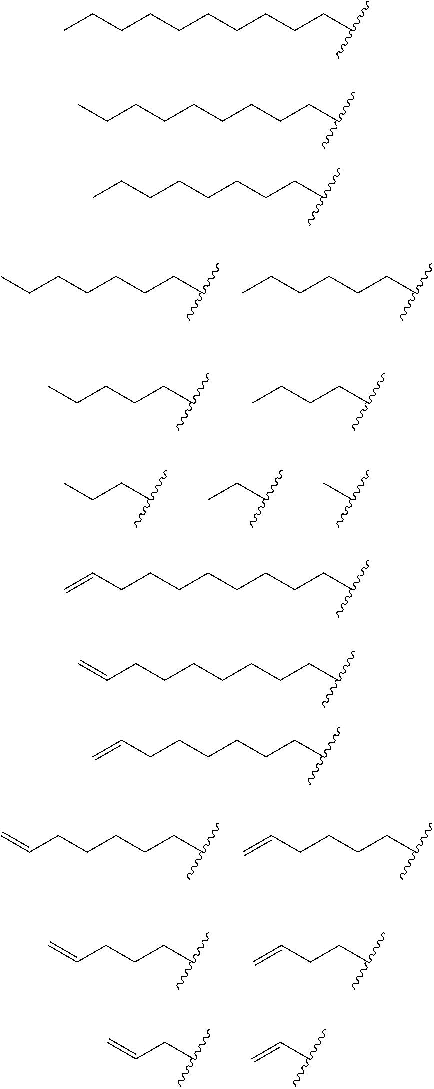

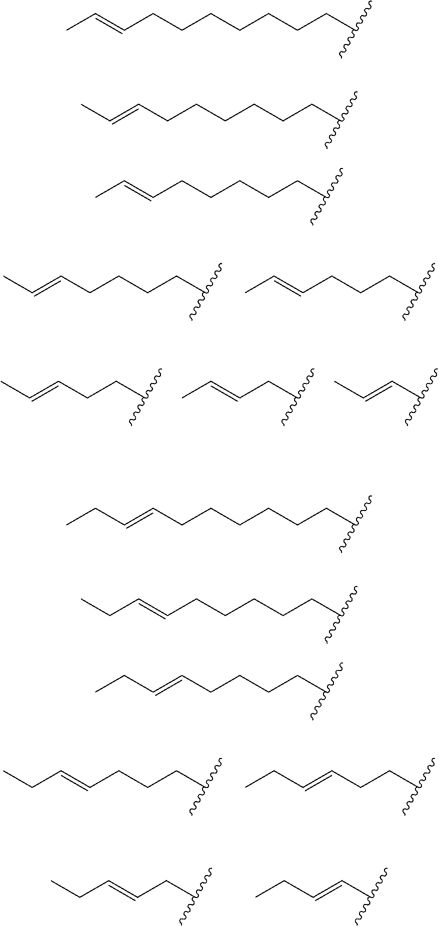

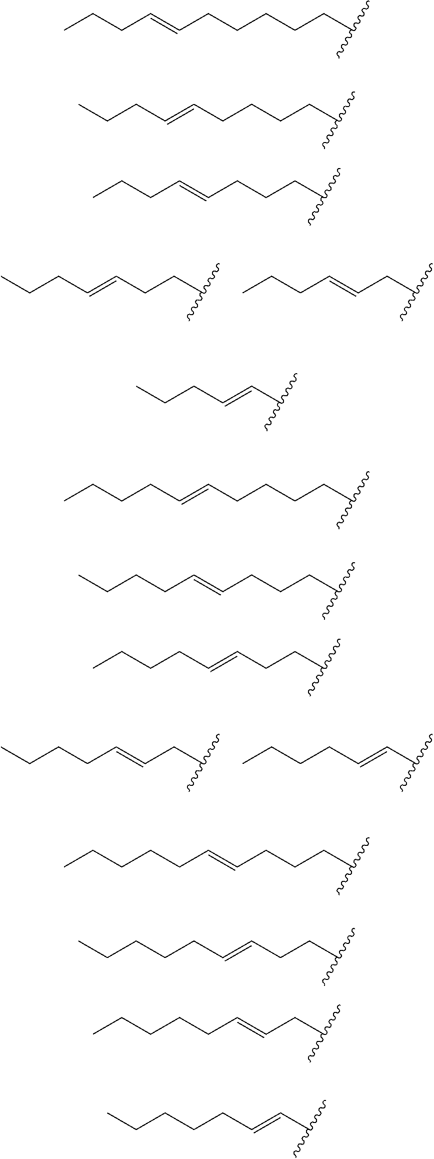

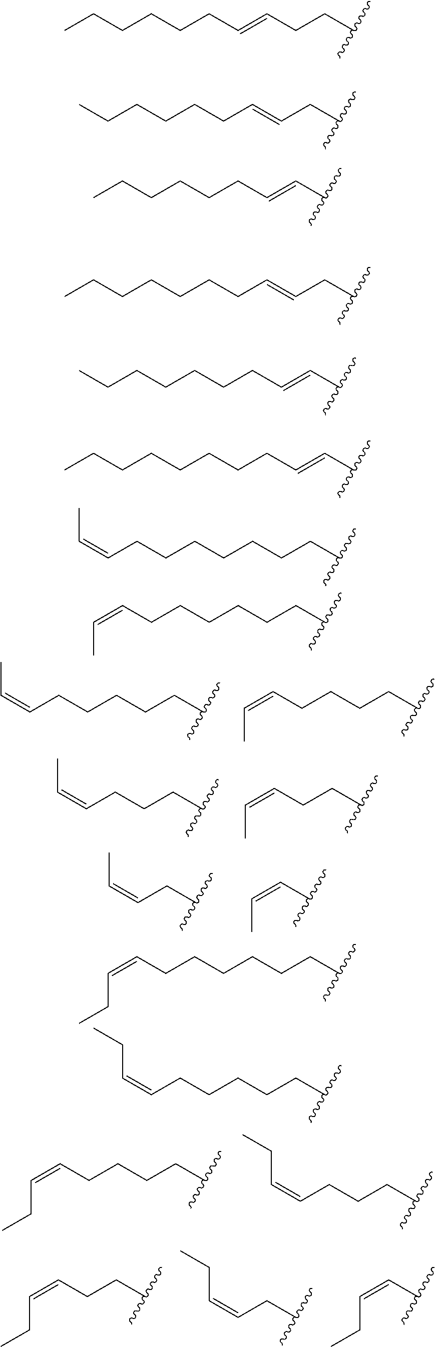

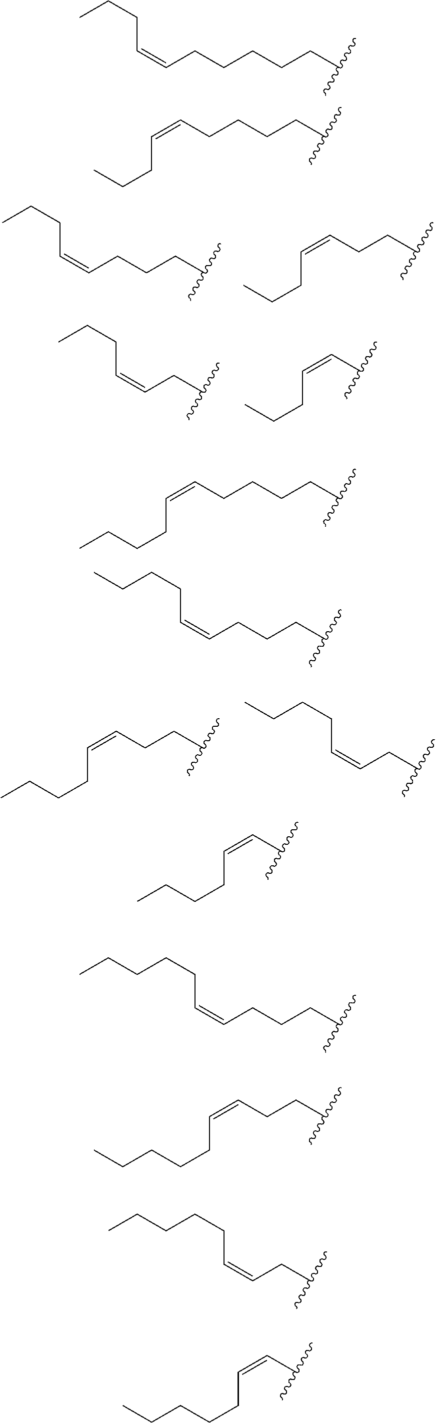

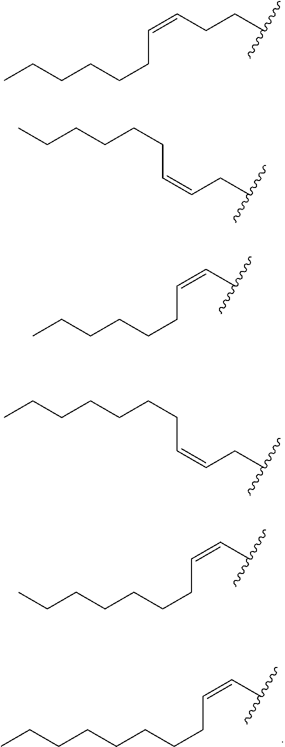

In some embodiments, T is selected from

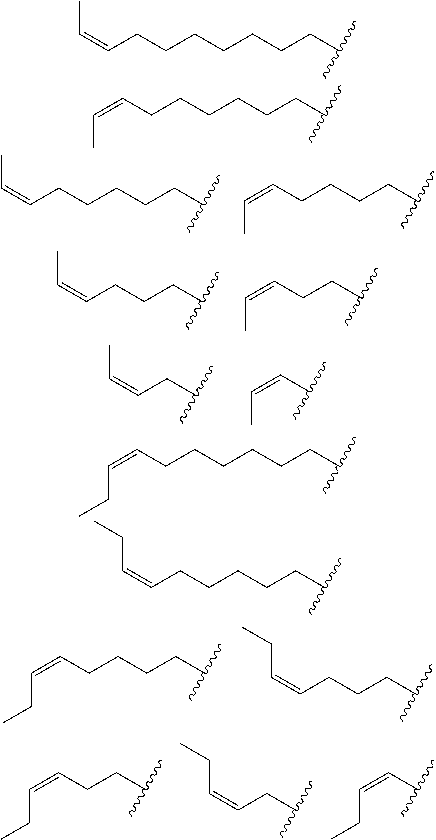

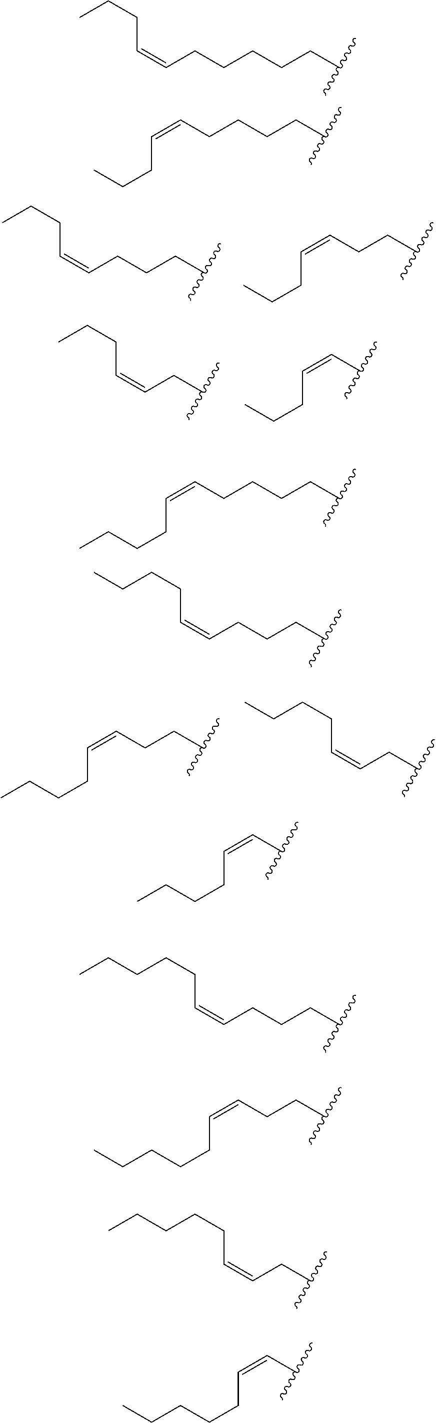

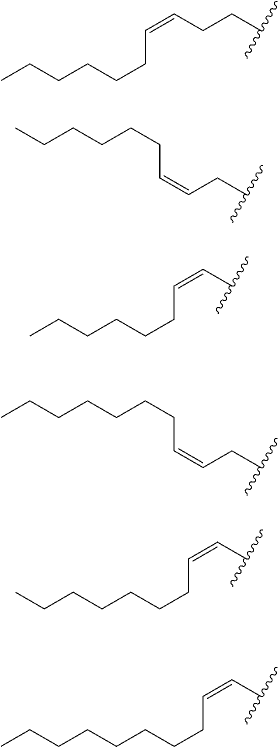

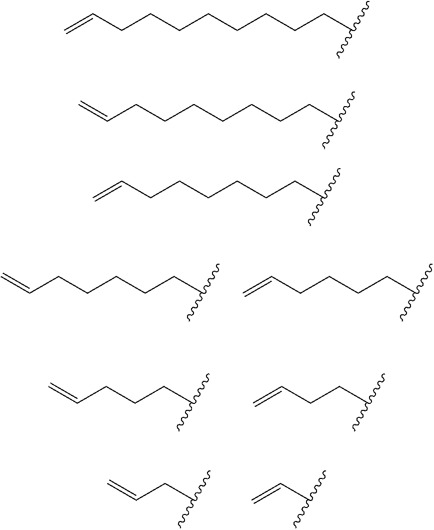

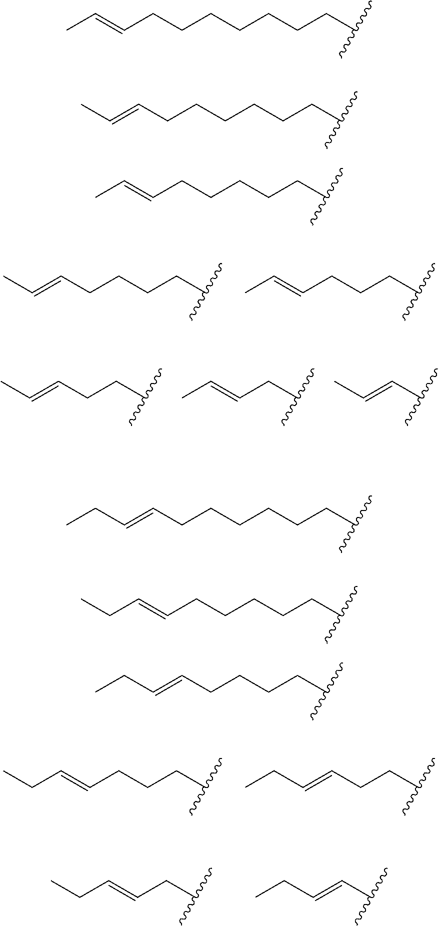

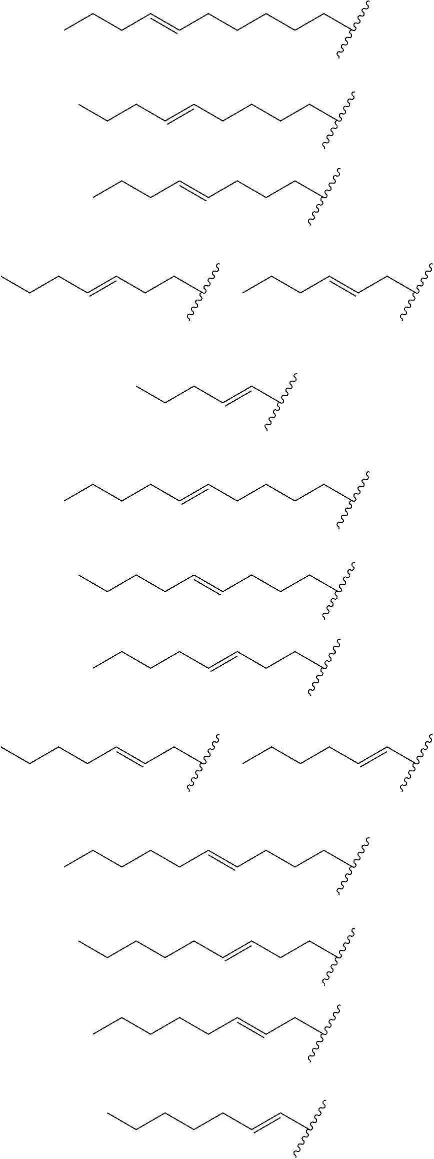

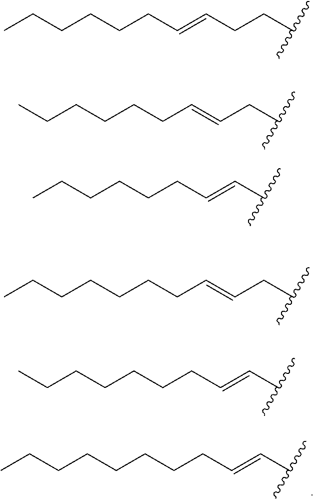

In some embodiments, T is selected from

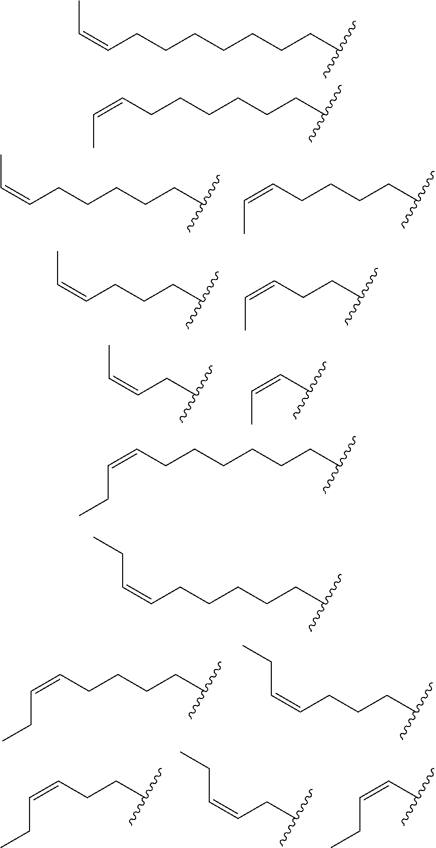

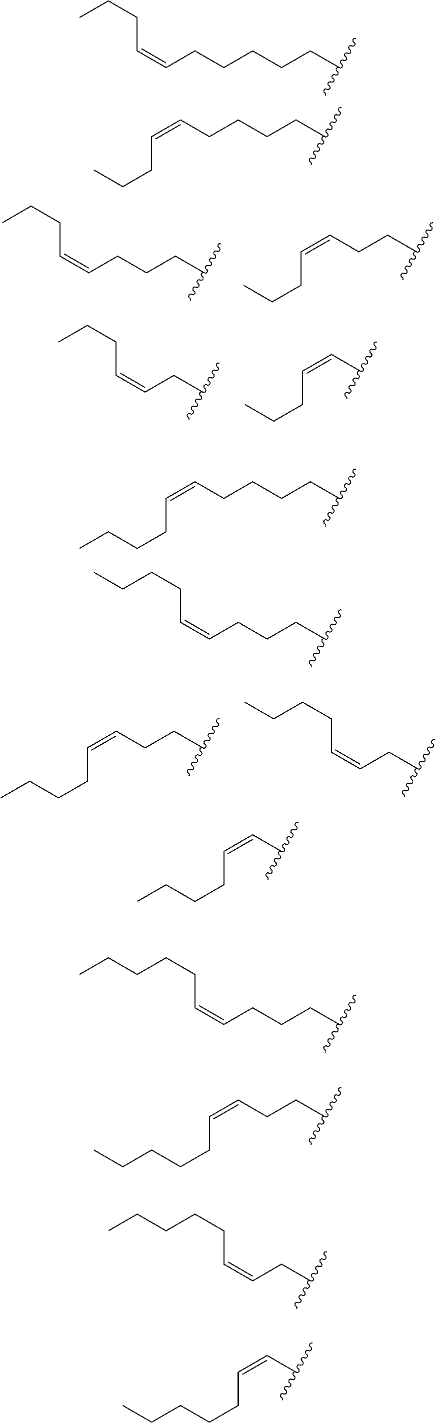

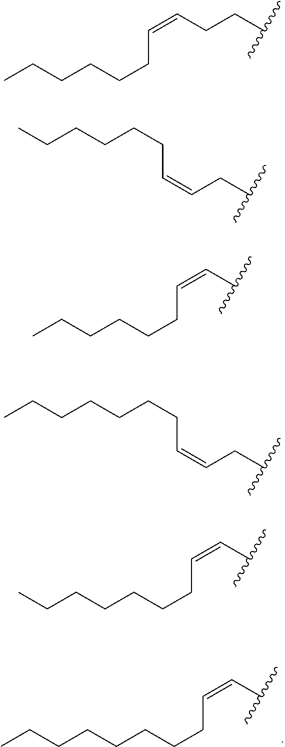

In some embodiments, T is selected from

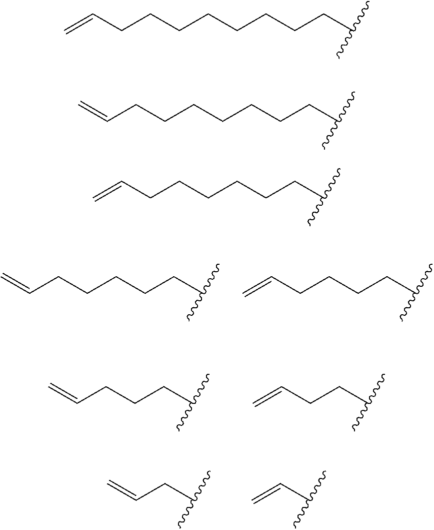

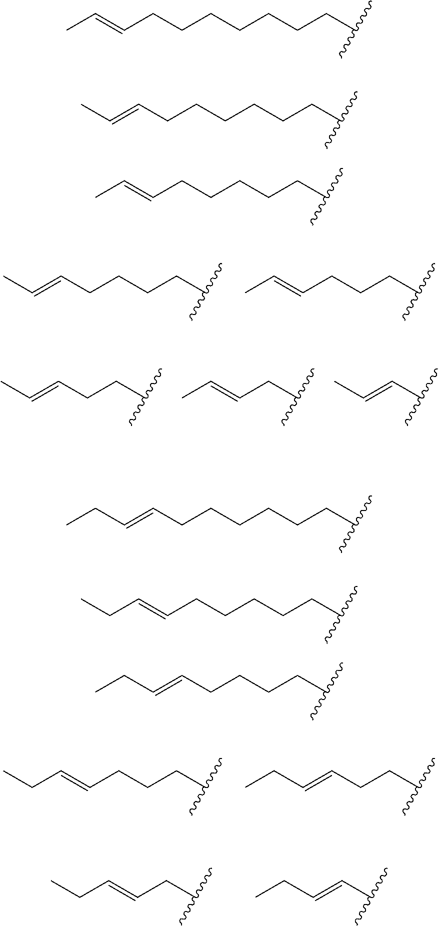

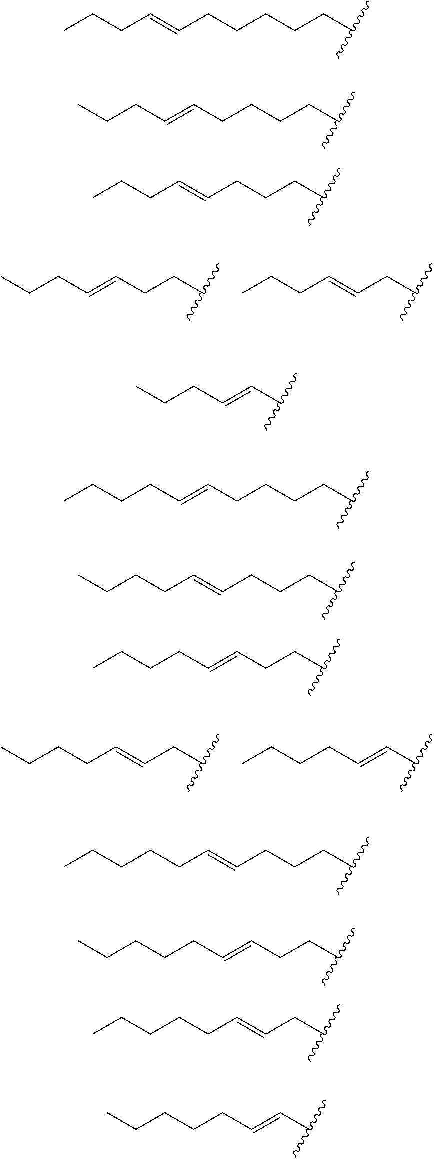

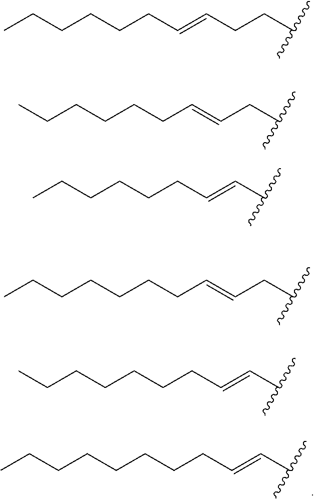

In some embodiments, T is selected from

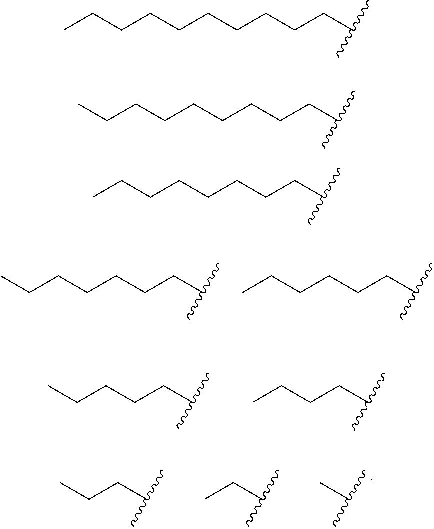

In some embodiments, T is

[symbol]

As defined herein, L is optionally substituted C₁-C₂₀ alkylene, optionally substituted C₂-C₂₀ alkenylene, or optionally substituted C₂-C₂₀ alkynylene. In some embodiments, L is unsubstituted C₁-C₂₀ alkylene, unsubstituted C₂-C₂₀ alkenylene, or unsubstituted C₂-C₂₀ alkynylene. In some embodiments, L is unsubstituted C₁-C₂₀ alkylene or unsubstituted C₂-C₂₀ alkenylene. In some embodiments, L is unsubstituted C₅-C₁₅ alkylene or unsubstituted C₅-C₁₅ alkenylene. In some embodiments, L is unsubstituted C₁-C₂₀ alkylene. In some embodiments, L is unsubstituted C₅-C₁₅ alkylene. In some embodiments, L is unsubstituted C₉-C₁₃ alkylene. In some embodiments, L is unsubstituted C₂-C₂₀ alkenylene. In some embodiments, L is unsubstituted C₅-C₁₅ alkenylene. In some embodiments, L is unsubstituted C₉-C₁₃ alkenylene. In some embodiments, L is unsubstituted C₂-C₂₀ alkenylene with one double bond. In some embodiments, L is unsubstituted C₅-C₁₅ alkenylene with one double bond. In some embodiments, L is unsubstituted C₉-C₁₃ alkenylene with one double bond.

In some embodiments, L is selected from:

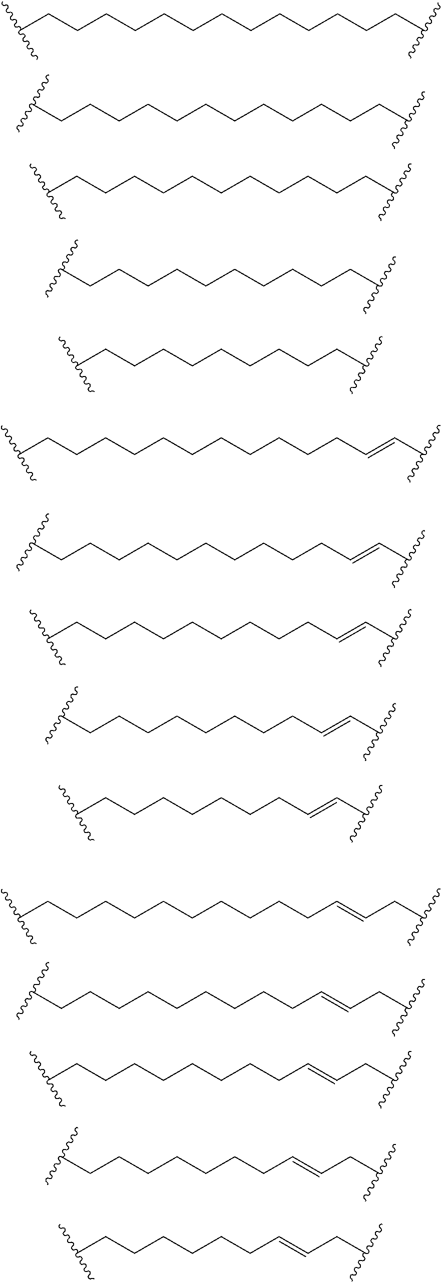

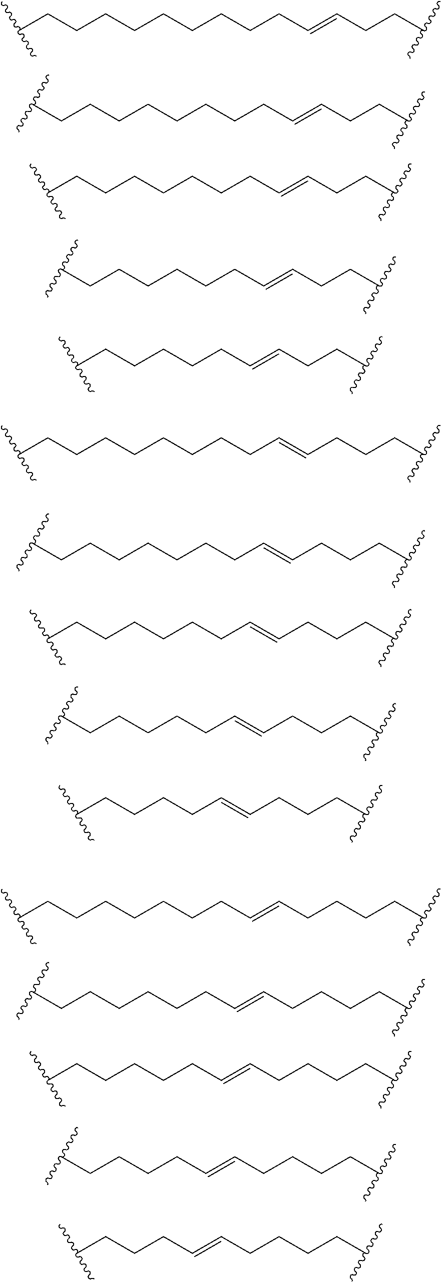

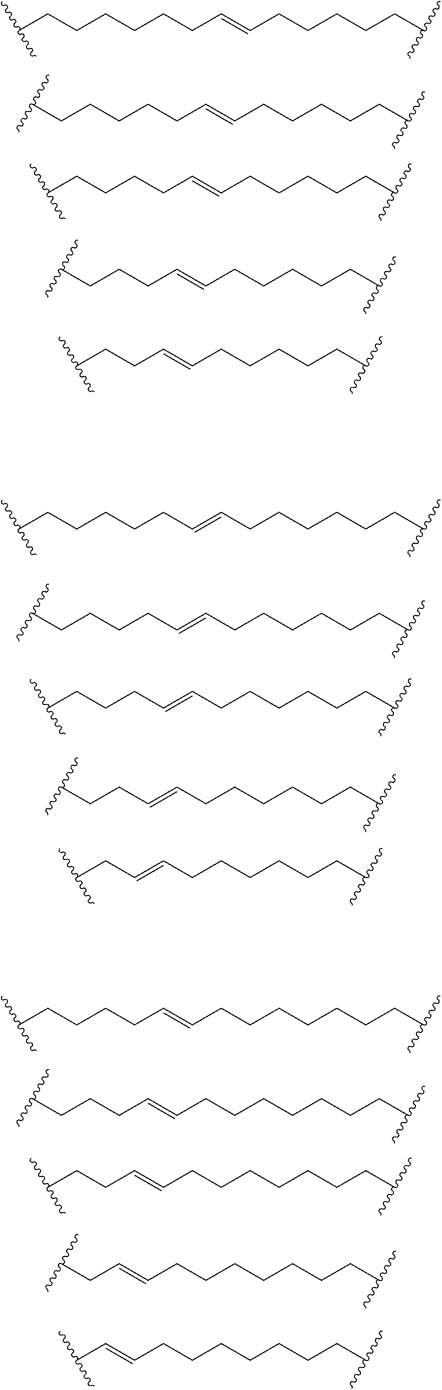

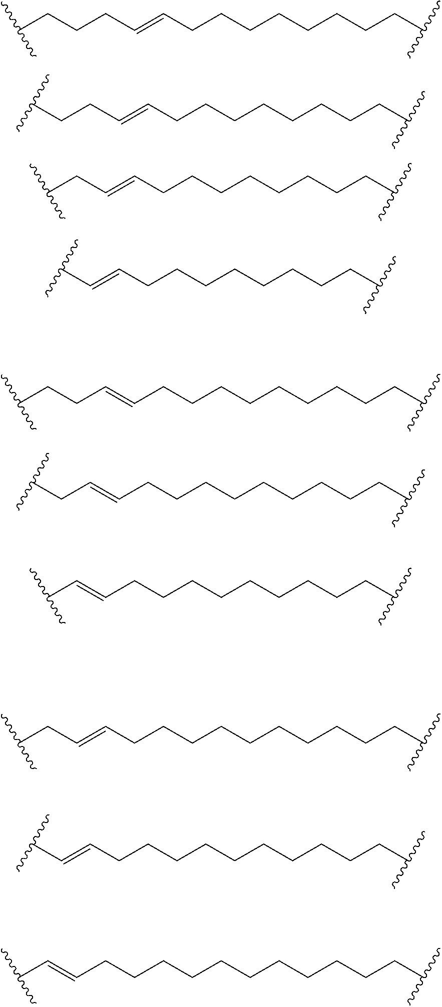

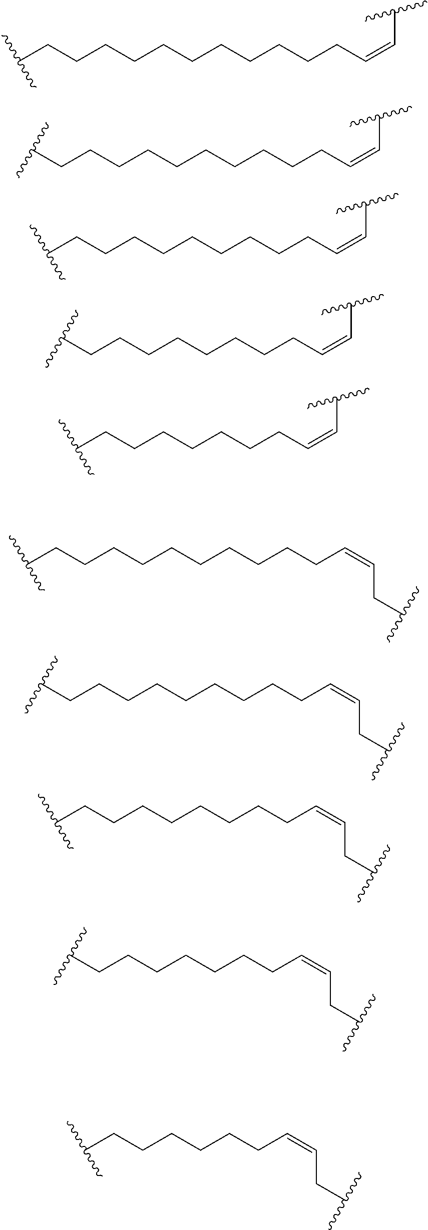

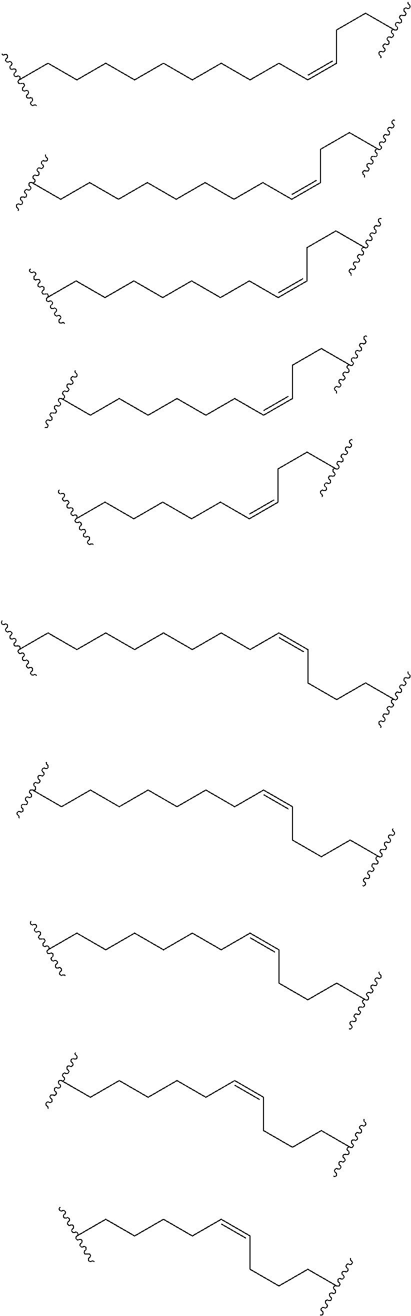

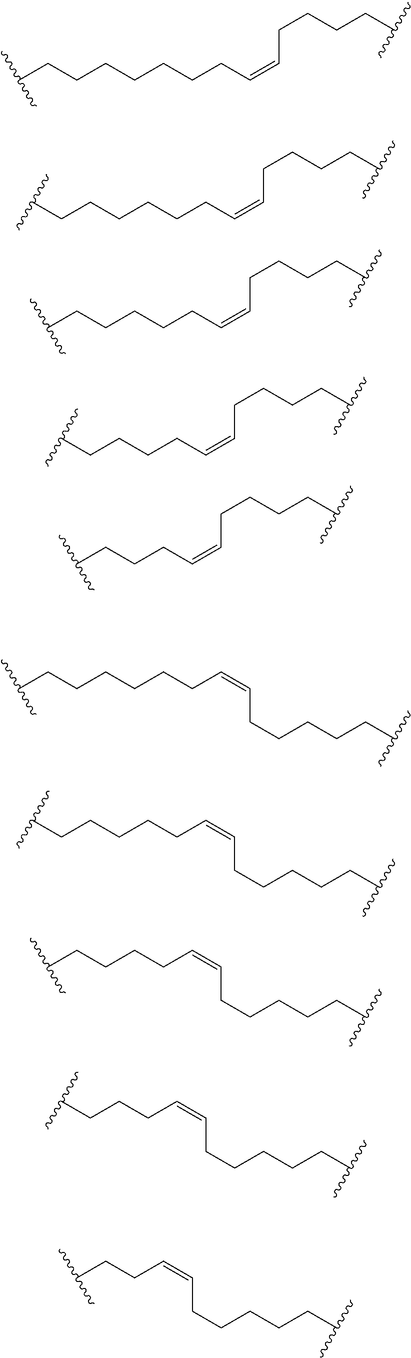

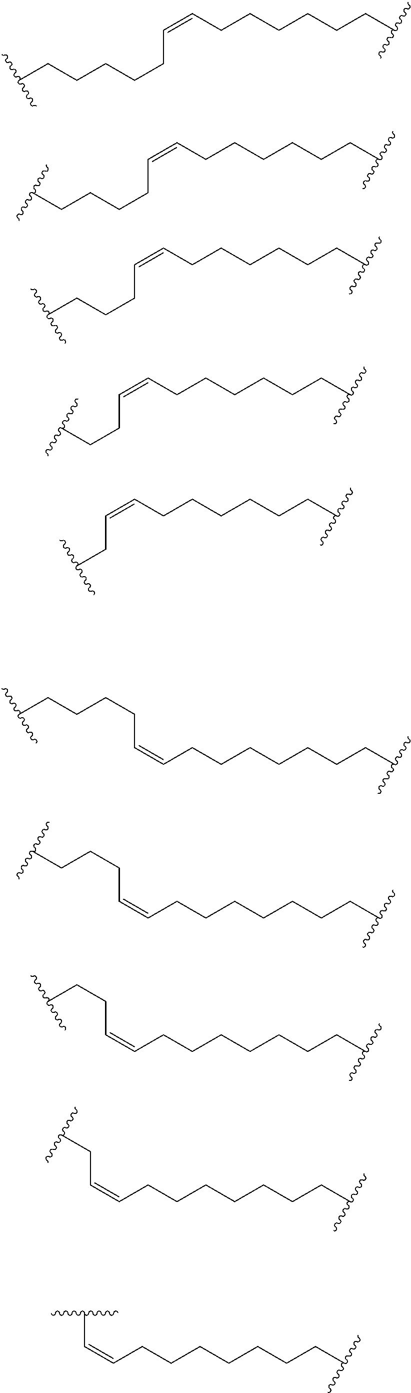

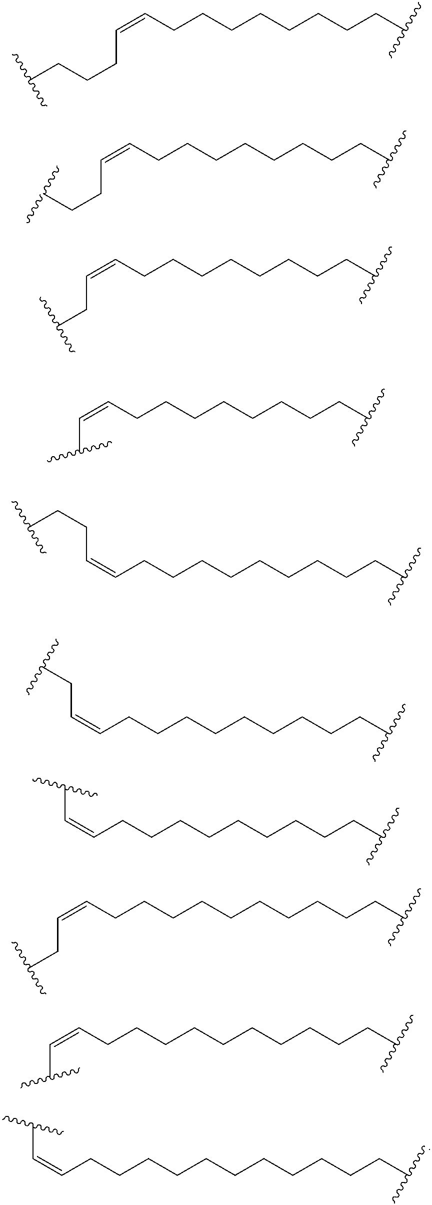

In some embodiments, L is selected from:

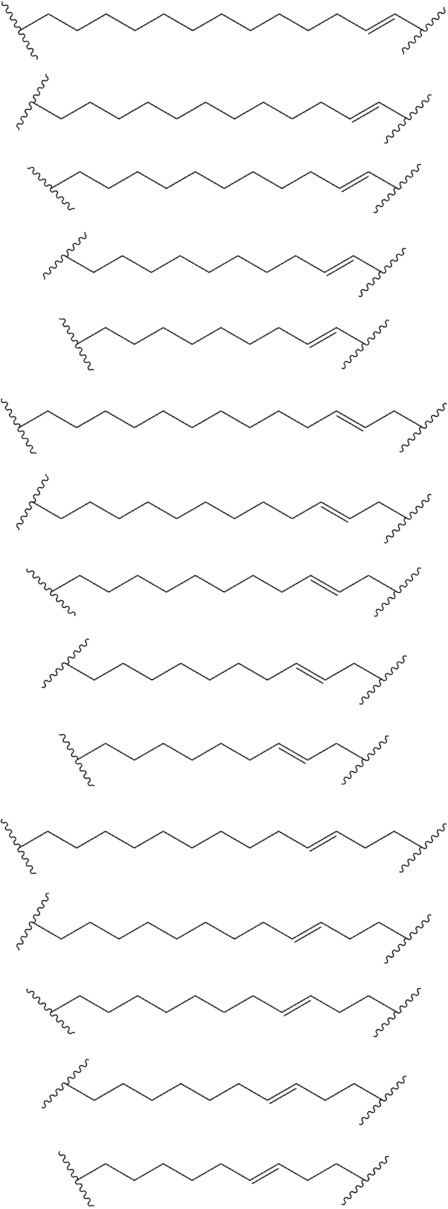

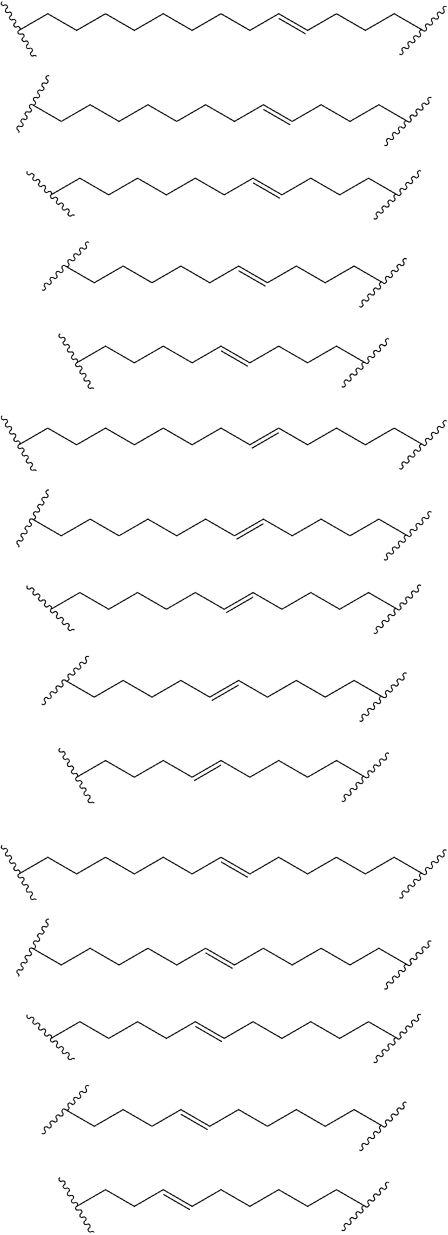

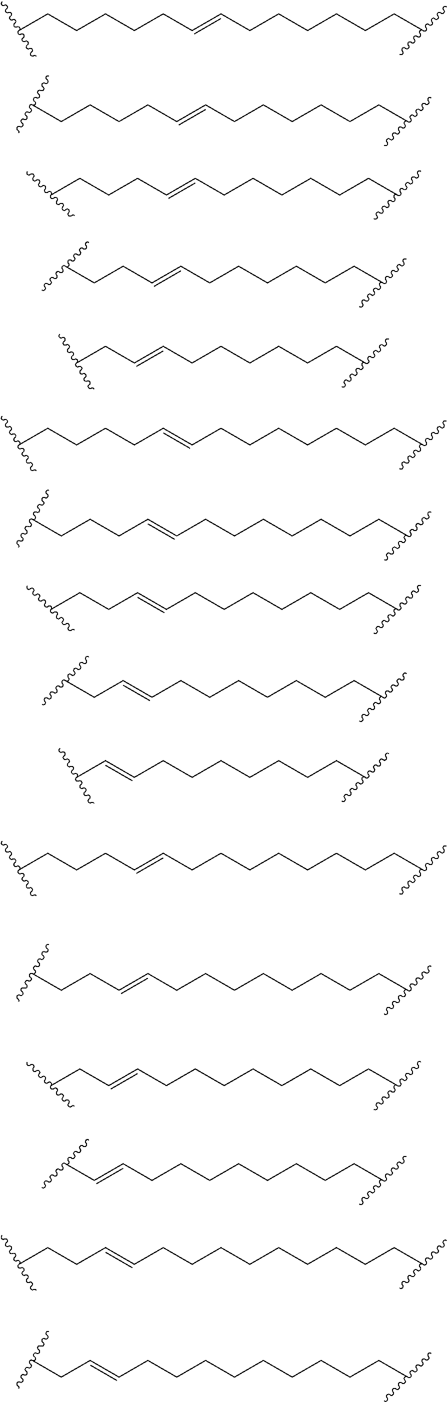

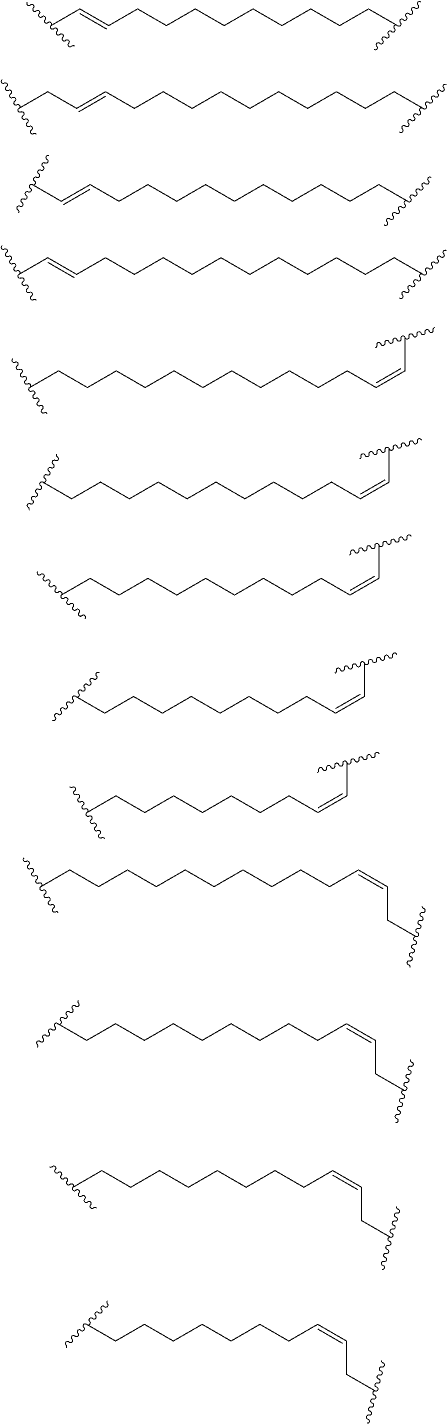

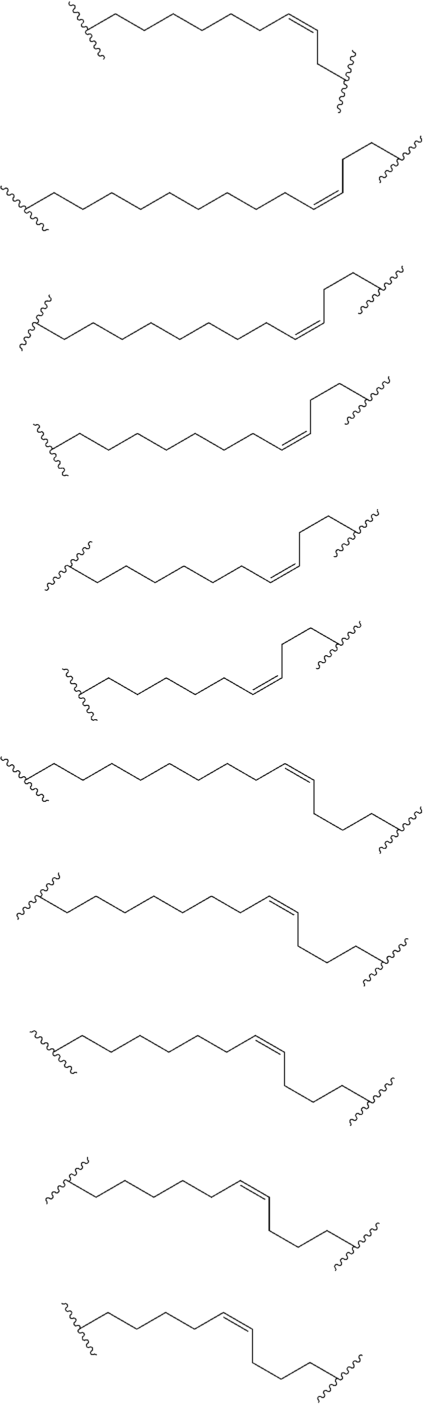

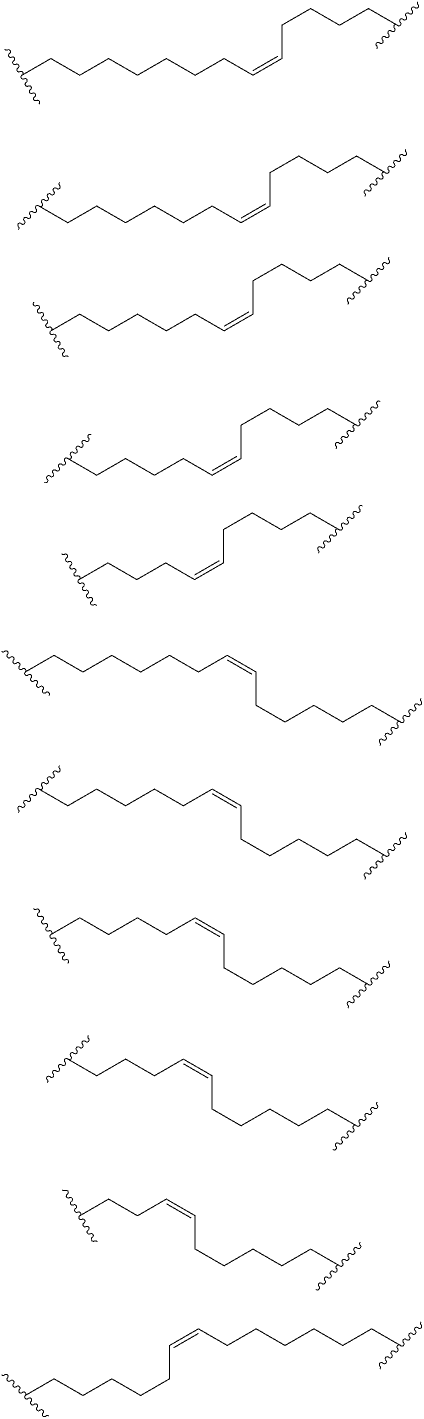

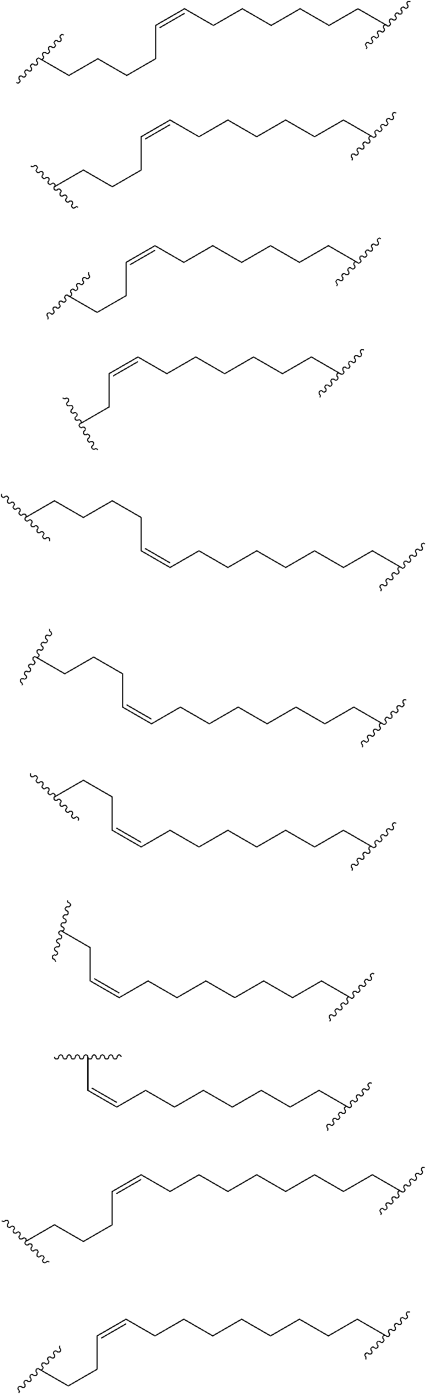

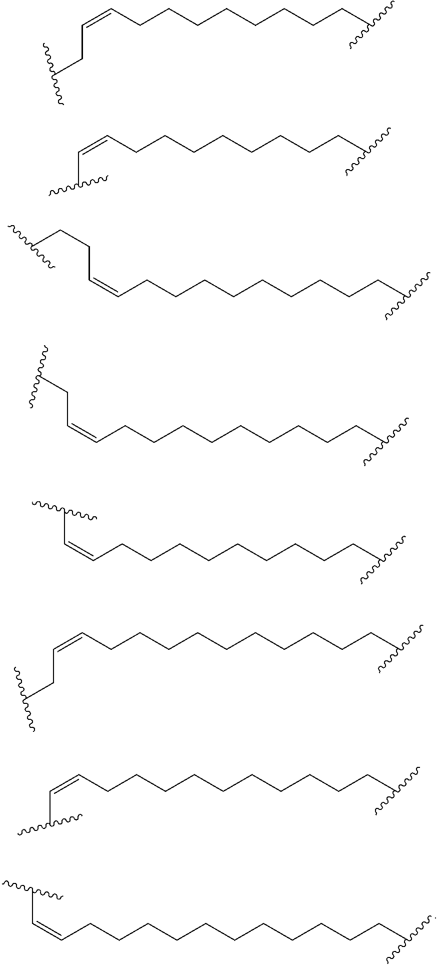

In some embodiments, L is selected from.

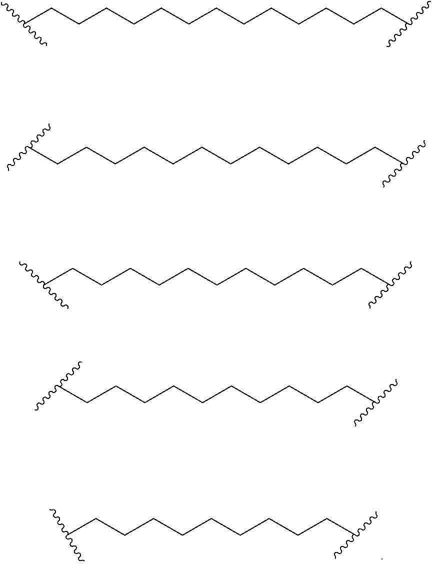 [SMILES: *CCCCCCCCCCCCC* ; *CCCCCCCCCCCC* ; *CCCCCCCCCCC* ; *CCCCCCCCCC* ; *CCCCCCCCC*]

In some embodiments, L is selected from

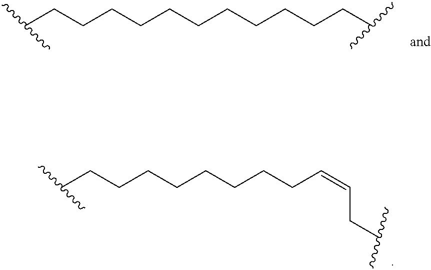 [SMILES: *CCCCCCCCCCC* ; *C/C=C\CCCCCCCC*]

In some embodiments, the compound of Formula (IV) is selected from an acrylate provided in FIG. 7. In some embodiments, the compound of Formula (IV) is selected from an acrylate provided in FIG. 10B. In some embodiments, the compound of Formula (IV) is selected from an acrylate provided in FIG. 7 or FIG. 10B.

As defined herein, R³ is optionally substituted aliphatic or optionally substituted heteroaliphatic. In some embodiments, R³ is optionally substituted aliphatic, —OR^{3O}, or —N(R^{3N})₂, wherein each instance of R^{3O} and R^{3N} is independently optionally substituted aliphatic. In certain embodiments, R³ is C₁-C₂₅ optionally substituted aliphatic, —OR^{3O}, or —N(R^{3N})₂, wherein each instance of R^{3O} and R^{3N} is independently optionally substituted C₁-C₂₅ aliphatic. In some embodiments, R³ is optionally substituted aliphatic or —OR^{3O}, wherein R^{3O} is optionally substituted aliphatic. In certain embodiments, R³ is C₁-C₂₅ optionally substituted aliphatic or —OR^{3O}, wherein R^{3O} is optionally substituted C₁-C₂₅ aliphatic.

In some embodiments, R³ is C₁-C₂₅ optionally substituted aliphatic. In some embodiments, R³ is C₁-C₂₅ substituted aliphatic. In certain embodiments, R³ is C₁-C₂₅ unsubstituted aliphatic. In some embodiments, R³ is C₁-C₂₅ optionally substituted alkyl. In some embodiments, R³ is C₁-C₂₅ substituted alkyl. In certain embodiments, R³ is C₁-C₂₅ unsubstituted alkyl. In some embodiments, R³ is C₁-C₂₅ alkyl substituted with C₃-C₈ unsubstituted cycloalkyl. In some embodiments, R³ is optionally substituted C₁-C₁₅ alkyl. In some embodiments, R³ is substituted C₁-C₁₅ alkyl. In some embodiments, R³ is unsubstituted C₁-C₁₅ alkyl. In some embodiments, R³ is C₁-C₁₅ alkyl substituted with C₃-C₈ unsubstituted cycloalkyl. In some embodiments, R³ is

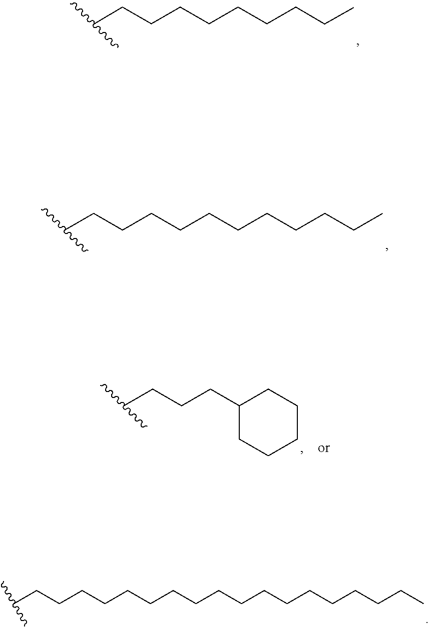

In some embodiments, R is

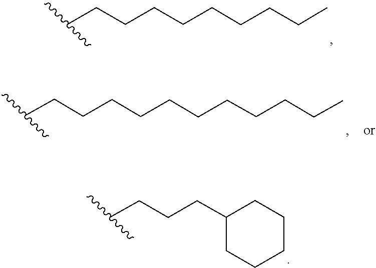

In some embodiments, R³ is

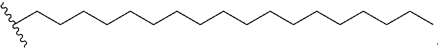 [SMILES: *CCCCCCCCCCCCCCCCCC]

In some embodiments, R³ is C₁-C₂₅ optionally substituted alkenyl. In some embodiments, R³ is C₁-C₂₅ substituted alkenyl. In certain embodiments, R³ is C₁-C₂₅ unsubstituted alkenyl. In some embodiments, R³ is C₁-C₂₅ optionally substituted alkenyl comprising one double bond. In some embodiments, R³ is C₁-C₂₅ optionally substituted alkenyl comprising at least two double bonds. In some embodiments, R³ is

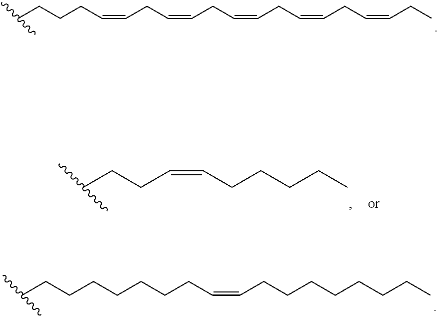

In some embodiments, R³ is

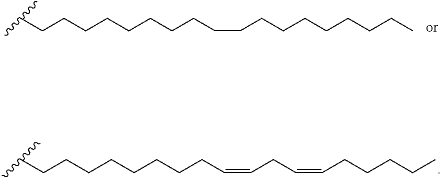 [SMILES: *CCCCCCCCCCCCCCCCCC ; *CCCCCCCC/C=C\C/C=C\CCCCC]

In some embodiments, R³ is

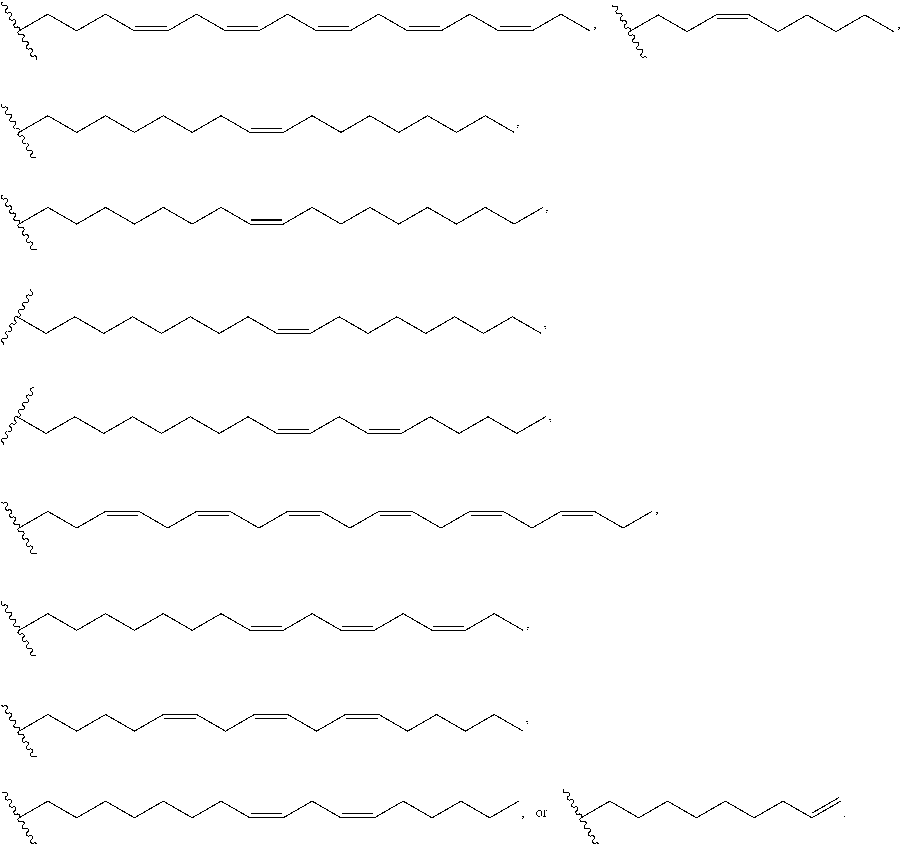

In some embodiments, R³ is C₁-C₂₅ optionally substituted alkynyl. In some embodiments, R³ is C₁-C₂₅ substituted alkynyl. In certain embodiments, R³ is C₁-C₂₅ unsubstituted alkynyl. In some embodiments, R³ is

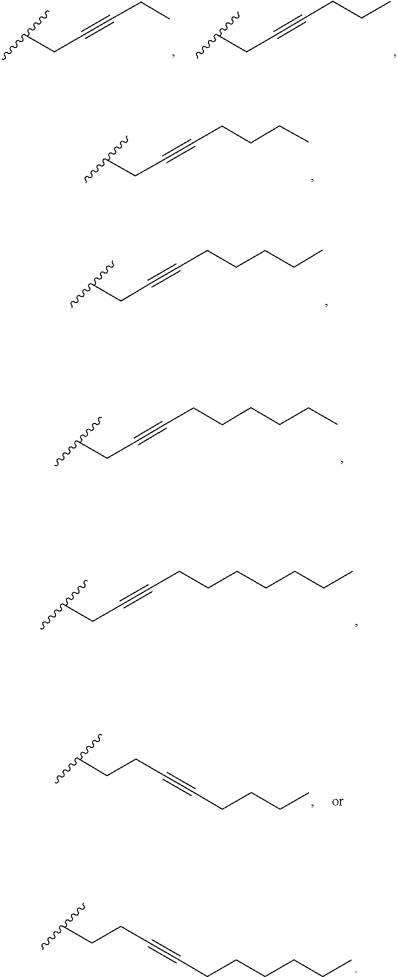

In certain embodiments, R³ is selected from the group consisting of

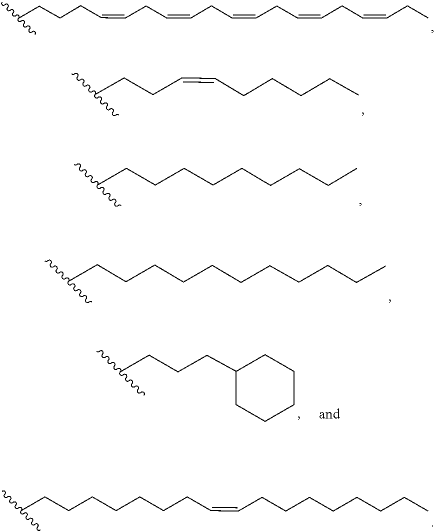

In certain embodiments, R³ is selected from the group consisting of

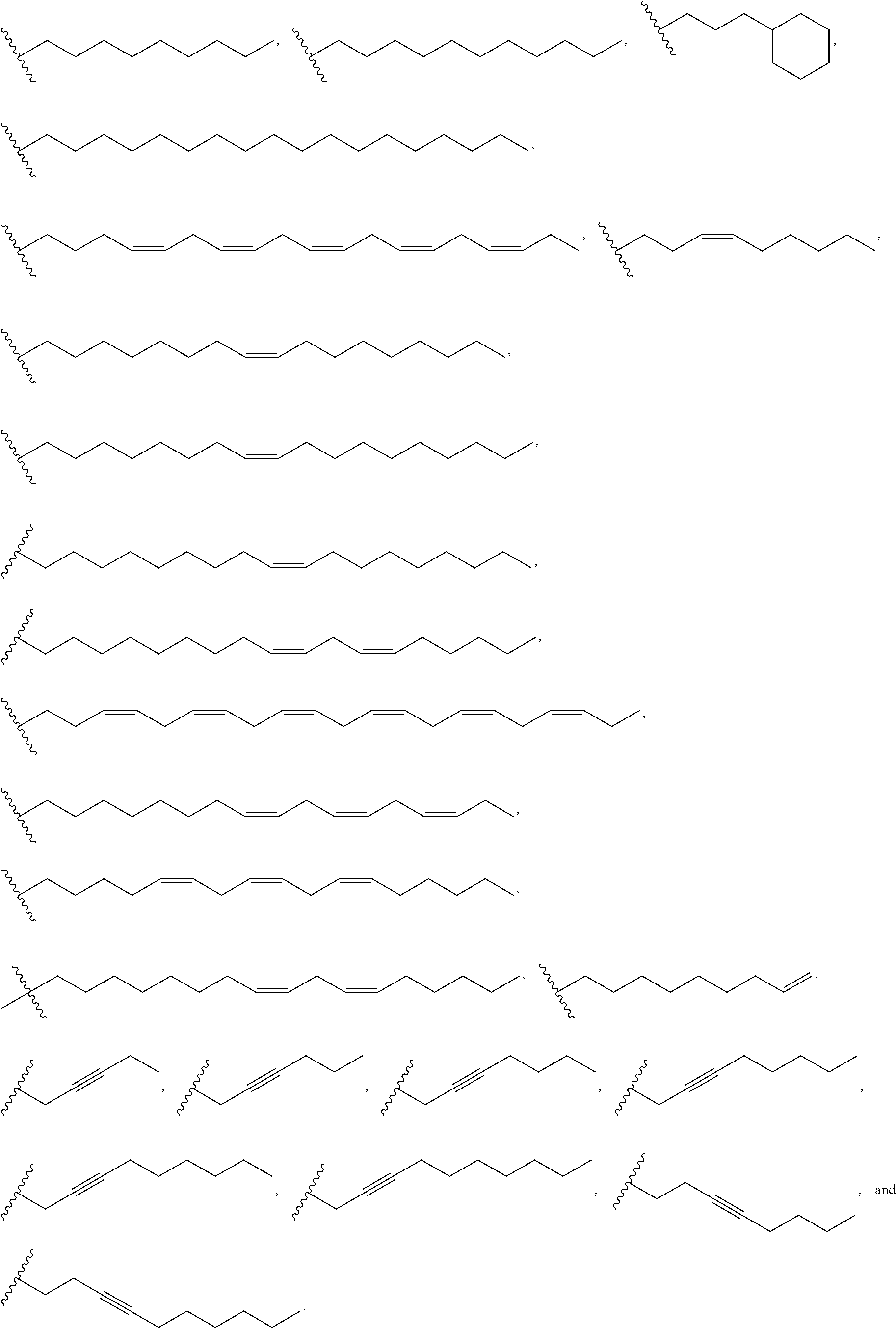

In certain embodiments, R³ is selected from the group consisting of

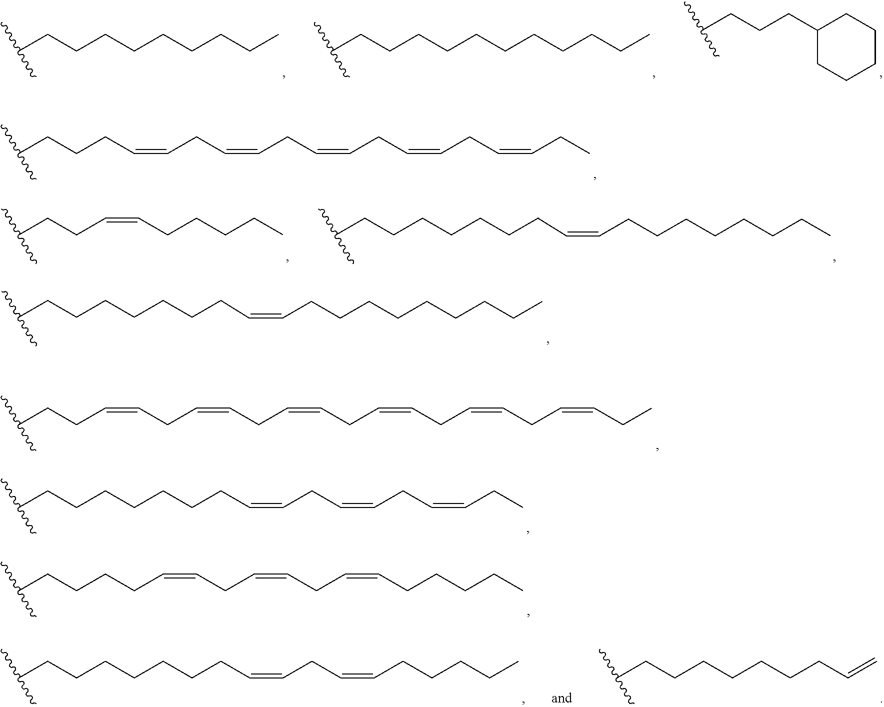

In some embodiments, R³ is optionally substituted C₁-C₂₅ heteroaliphatic. In certain embodiments, R³ is —OR^{3O} or —N(R^{3N})₂, wherein each instance of R^{3O} and R^{3N} is independently optionally substituted aliphatic. In certain embodiments, R³ is —OR^{3O} or —N(R^{3N})₂, wherein each instance of R^{3O} and R^{3N} is independently optionally substituted C₁-C₂₅ aliphatic.

In certain embodiments, R³ is —OR^{3O}, wherein R^{3O} is optionally substituted C₁-C₂₅ aliphatic.

In some embodiments, R^{3O} is C₁-C₂₅ optionally substituted aliphatic. In some embodiments, R^{3O} is C₁-C₂₅ substituted aliphatic. In certain embodiments, R^{3O} is C₁-C₂₅ unsubstituted aliphatic. In some embodiments, R^{3O} is C₁-C₂₅ optionally substituted alkyl. In some embodiments, R^{3O} is C₁-C₂₅ substituted alkyl. In certain embodiments, R^{3O} is C₁-C₂₅ unsubstituted alkyl. In some embodiments, R^{3O} is

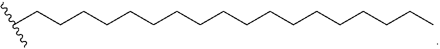 [SMILES: *CCCCCCCCCCCCCCCCCC]

In some embodiments, R^{3O} is C₁-C₂₅ optionally substituted alkenyl. In some embodiments, R^{3O} is C₁-C₂₅ substituted alkenyl. In certain embodiments, R^{3O} is C₁-C₂₅ unsubstituted alkenyl. In some embodiments, R^{3O} is C₁-C₂₅ optionally substituted alkenyl comprising one double bond. In some embodiments, R^{3O} is C₁-C₂₅ optionally substituted alkenyl comprising at least two double bonds. In some embodiments, R^{3O} is

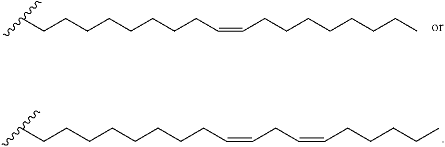 [SMILES: *CCCCCCCC/C=C\CCCCCCCC ; *CCCCCCCC/C=C\C/C=C\CCCCC]

In some embodiments, R^{3O} is C₁-C₂₅ optionally substituted alkynyl. In some embodiments, R^{3O} is C₁-C₂₅ substituted alkynyl. In certain embodiments, R^{3O} is C₁-C₂₅ unsubstituted alkynyl. In some embodiments, R^{3O} is

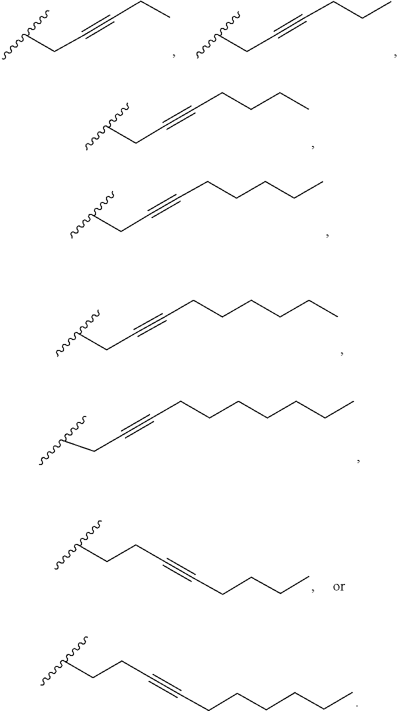

In some embodiments, R^{3O} is selected from the group consisting of

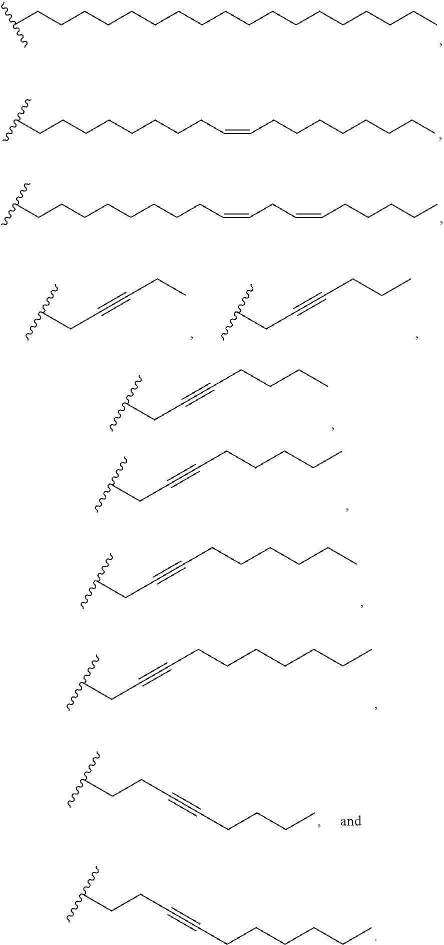

In certain embodiments, R³ is —N(R^{3N})₂, wherein each instance of R^{3N} is independently optionally substituted C₁-C₂₅ aliphatic.

In some embodiments, R^{3N} is C₁-C₂₅ optionally substituted aliphatic. In some embodiments, R^{3N} is C₁-C₂₅ substituted aliphatic. In certain embodiments, R^{3N} is C₁-C₂₅ unsubstituted aliphatic. In some embodiments, R^{3N} is C₁-C₂₅ optionally substituted alkyl. In some embodiments, R^{3N} is C₁-C₂₅ substituted alkyl.

In some embodiments, R^{3N} is C₁-C₂₅ optionally substituted alkenyl. In some embodiments, R^{3N} is C₁-C₂₅ substituted alkenyl. In certain embodiments, R^{3N} is C₁-C₂₅ unsubstituted alkenyl. In some embodiments, R^{3N} is C₁-C₂₅ optionally substituted alkenyl comprising one double bond. In some embodiments, R^{3N} is C₁-C₂₅ optionally substituted alkenyl comprising at least two double bonds.

In some embodiments, R^{3N} is C₁-C₂₅ optionally substituted alkynyl. In some embodiments, R^{3N} is C₁-C₂₅ substituted alkynyl. In certain embodiments, R^{3N} is C₁-C₂₅ unsubstituted alkynyl.

As defined herein, X is —OR¹, —SR¹, or —NR¹R². In certain embodiments, X is —OR¹, or —SR¹. In some embodiments, X is —SR¹ or —NR¹R². In certain embodiments, X is —OR¹ or —NR¹R². In some embodiments, X is —OR¹. In certain embodiments, X is —SR¹. In some embodiments, X is —NR¹R².

In some embodiments, H—X is H—NR¹R². In certain embodiments, H—X is selected from an amine provided in FIG. 3 or FIG. 10A.

As defined herein, R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group; or R¹ and R² are joined together with the intervening atoms to form an optionally substituted heterocyclyl.

In certain embodiments, R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group. In some embodiments, R¹ is —H, optionally substituted alkyl, optionally substituted heteroalkyl, optionally substituted aryl, optionally substituted heteroaryl, optionally substituted cycloalkyl, or optionally substituted heterocyclyl. In certain embodiments, R¹ is optionally substituted alkyl, optionally substituted heteroalkyl, optionally substituted aryl, optionally substituted heteroaryl, optionally substituted cycloalkyl, or optionally substituted heterocyclyl. In some embodiments, R¹ is —H, optionally substituted alkyl, optionally substituted heteroalkyl, optionally substituted cycloalkyl, or optionally substituted heterocyclyl. In some embodiments, R¹ is optionally substituted alkyl, optionally substituted heteroalkyl, optionally substituted cycloalkyl, or optionally substituted heterocyclyl. In certain embodiments, R¹ is —H, optionally substituted alkyl, optionally substituted heteroalkyl, or optionally substituted heterocyclyl. In certain embodiments, R¹ is optionally substituted alkyl, optionally substituted heteroalkyl, or optionally substituted heterocyclyl.

In certain embodiments, R¹ is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R¹ is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R¹ is optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In some embodiments, R¹ is optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In certain embodiments, R¹ is —H, optionally substituted C₁-C₂₅ alkyl, optionally substituted C₁-C₂₅ heteroalkyl, or optionally substituted 3- to 8-membered heterocyclyl. In certain embodiments, R¹ is optionally substituted C₁-C₂₅ alkyl, optionally substituted C₁-C₂₅ heteroalkyl, or optionally substituted 3- to 8-membered heterocyclyl.

In certain embodiments, R¹ is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R¹ is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R¹ is optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In some embodiments, R¹ is optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In certain embodiments, R¹ is —H, optionally substituted C₁-C₂₀ alkyl, optionally substituted C₁-C₂₀ heteroalkyl, or optionally substituted 3- to 8-membered heterocyclyl. In certain embodiments, R¹ is optionally substituted C₁-C₂₀ alkyl, optionally substituted C₁-C₂₀ heteroalkyl, or optionally substituted 3- to 8-membered heterocyclyl.

In some embodiments, R¹ is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃₋₈ cycloalkyl, optionally substituted 3- to 8-membered heterocyclyl, or a protecting group. In some embodiments, R¹ is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-s cycloalkyl, or optionally substituted 3- to 8-membered heterocyclyl. In certain embodiments, R¹ is optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R¹ is —H, optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In some embodiments, R¹ is optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In certain embodiments, R¹ is —H, optionally substituted C₁-C₁₀ alkyl, optionally substituted C₁-C₁₀ heteroalkyl, or optionally substituted 3- to 8-membered heterocyclyl. In certain embodiments, R¹ is optionally substituted C₁-C₁₀ alkyl, optionally substituted C₁-C₁₀ heteroalkyl, or optionally substituted 3- to 8-membered heterocyclyl.

In some embodiments, R¹ is a protecting group. In some embodiments the protecting group is a nitrogen protecting group, oxygen protecting group, or sulfur protecting group. In some embodiments, the protecting group is a nitrogen protecting group. In some embodiments the protecting group is an oxygen protecting group. In some embodiments, the protecting group is a sulfur protecting group. In some embodiments, R¹ is —H. In certain embodiments, R¹ is —H or a protecting group. In some embodiments, R¹ is —H or a nitrogen protecting group, an oxygen protecting group, or a sulfur protecting group. In some embodiments, R¹ is —H or a nitrogen protecting group.

In certain embodiments, R¹ is optionally substituted alkyl. In some embodiments, R¹ is optionally substituted C₁-C₂₅ alkyl. In certain embodiments, R¹ is optionally substituted C₁-C₂₀ alkyl. In some embodiments, R¹ is optionally substituted C₁-C₁₅ alkyl. In certain embodiments, R¹ is optionally substituted C₁-C₁₀ alkyl. In some embodiments, R¹ is optionally substituted C₁-C₆ alkyl. In certain embodiments, R¹ is substituted alkyl. In some embodiments, R¹ is substituted C₁-C₂₅ alkyl. In certain embodiments, R¹ is substituted C₁-C₂₀ alkyl. In some embodiments, R¹ is substituted C₁-C₁₅ alkyl. In certain embodiments, R¹ is substituted C₁-C₁₀ alkyl. In some embodiments, R¹ is substituted C₁-C₆ alkyl. In certain embodiments, R¹ is unsubstituted alkyl. In some embodiments, R¹ is unsubstituted C₁-C₂₅ alkyl. In certain embodiments, R¹ is unsubstituted C₁-C₂₀ alkyl. In some embodiments, R¹ is unsubstituted C₁-C₁₅ alkyl. In certain embodiments, R¹ is unsubstituted C₁-C₁₀ alkyl. In some embodiments, R¹ is unsubstituted C₁-C₆ alkyl. In certain embodiments, R¹ is methyl, ethyl, propyl, or butyl. In some embodiments, R¹ is methyl or ethyl. In certain embodiments, R¹ is methyl. In some embodiments, R¹ is ethyl.

In certain embodiments, R¹ is optionally substituted heteroalkyl. In some embodiments, R¹ is optionally substituted C₁-C₂₅ heteroalkyl. In certain embodiments, R¹ is optionally substituted C₁-C₂₀ heteroalkyl. In some embodiments, R¹ is optionally substituted C₁-C₁₅ heteroalkyl. In certain embodiments, R¹ is optionally substituted C₁-C₁₀ heteroalkyl. In some embodiments, R¹ is optionally substituted C₁-C₆ heteroalkyl. In certain embodiments, R¹ is substituted heteroalkyl. In some embodiments, R¹ is substituted C₁-C₂₅ heteroalkyl. In certain embodiments, R¹ is substituted C₁-C₂₀ heteroalkyl. In some embodiments, R¹ is substituted C₁-C₁₅ heteroalkyl. In certain embodiments, R¹ is substituted C₁-C₁₀ heteroalkyl. In some embodiments, R¹ is substituted C₁-C₆ heteroalkyl. In certain embodiments, R¹ is unsubstituted heteroalkyl. In some embodiments, R¹ is unsubstituted C₁-C₂₅ heteroalkyl. In certain embodiments, R¹ is unsubstituted C₁-C₂₀ heteroalkyl. In some embodiments, R¹ is unsubstituted C₁-C₁₅ heteroalkyl. In certain embodiments, R¹ is unsubstituted C₁-C₁₀ heteroalkyl. In some embodiments, R¹ is unsubstituted C₁-C₆ heteroalkyl. In some embodiments, R¹ is

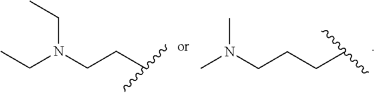

In some embodiments, R¹ is optionally substituted C₆-C₁₄ aryl. In some embodiments, R¹ is substituted C₆-C₁₄ aryl. In some embodiments, R¹ is unsubstituted C₆-C₁₄ aryl. In certain embodiments, R¹ is optionally substituted C₆-C₁₀ aryl. In certain embodiments, R¹ is substituted C₆-C₁₀ aryl. In certain embodiments, R¹ is unsubstituted C₆-C₁₀ aryl. In some embodiments, R¹ is optionally substituted phenyl or optionally substituted naphthyl. In certain embodiments, R¹ is optionally substituted phenyl. In some embodiments, R¹ is optionally substituted naphthyl.

In some embodiments, R¹ is optionally substituted 5- to 14-membered heteroaryl. In some embodiments, R¹ is substituted 5- to 14-membered heteroaryl. In some embodiments, R¹ is unsubstituted 5- to 14-membered heteroaryl. In some embodiments, R¹ is optionally substituted 5- to 10-membered heteroaryl. In some embodiments, R¹ is substituted 5- to 10-membered heteroaryl. In some embodiments, R¹ is unsubstituted 5- to 10-membered heteroaryl. In some embodiments, R¹ is optionally substituted pyridinyl.

In some embodiments, R¹ is optionally substituted C₃-C₈ cycloalkyl. In some embodiments, R¹ is substituted C₃-C₈ cycloalkyl. In some embodiments, R¹ is unsubstituted C₃-C₈ cycloalkyl. In some embodiments, R¹ is C₃-C₈ cycloalkyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R¹ is optionally substituted 3- to 8-membered heterocyclyl. In some embodiments, R¹ is substituted 3- to 8-membered heterocyclyl. In some embodiments, R¹ is unsubstituted 3- to 8-membered heterocyclyl. In some embodiments, R¹ is 3- to 8-membered heterocyclyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R¹ is not

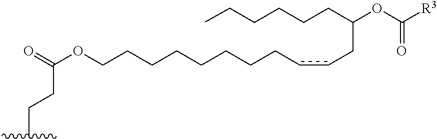

In certain embodiments, R¹ is —H, -Me, or -Et. In some embodiments, R¹ is —H, -Me,

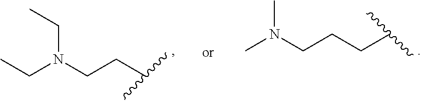

In certain embodiments, R¹ is —H, -Me, or -Et,

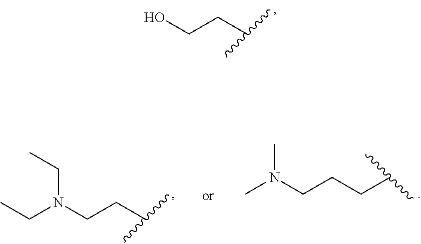

In some embodiments, R¹ is —H, -Me, -Et,

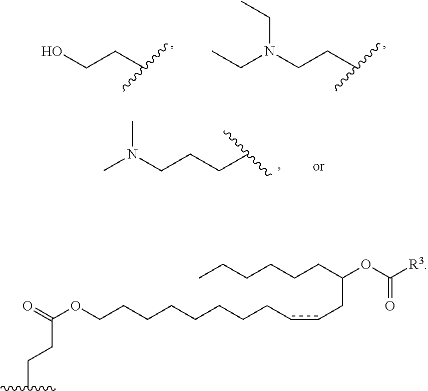

In some embodiments, R¹ is —H, -Me, -Et,

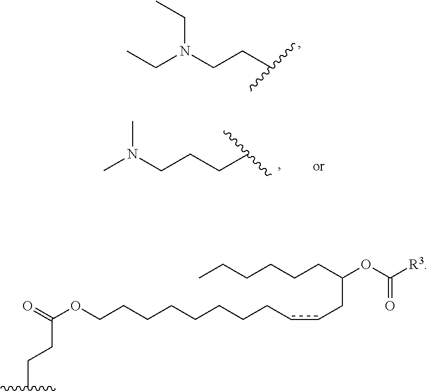

In some embodiments, R¹ is —H, -Me, -Et, or

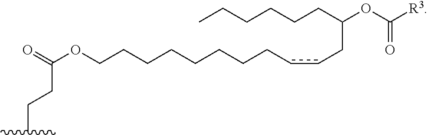

In some embodiments, R¹ is

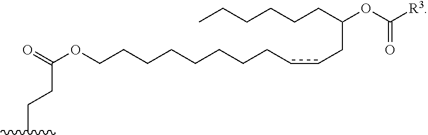

As defined herein, R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or R¹ and R² are joined together with the intervening atoms to form an optionally substituted heterocyclyl.

In certain embodiments, R² is —H, optionally substituted alkyl, optionally substituted heteroalkyl, optionally substituted aryl, optionally substituted heteroaryl, optionally substituted cycloalkyl, optionally substituted heterocyclyl, or a protecting group. In some embodiments, R² is —H, optionally substituted alkyl, optionally substituted heteroalkyl, optionally substituted aryl, optionally substituted heteroaryl, optionally substituted cycloalkyl, or optionally substituted heterocyclyl. In certain embodiments, R² is optionally substituted alkyl, optionally substituted heteroalkyl, optionally substituted aryl, optionally substituted heteroaryl, optionally substituted cycloalkyl, or optionally substituted heterocyclyl. In some embodiments, R² is —H, optionally substituted alkyl, optionally substituted heteroalkyl, optionally substituted cycloalkyl, or optionally substituted heterocyclyl. In some embodiments, R² is optionally substituted alkyl, optionally substituted heteroalkyl, optionally substituted cycloalkyl, or optionally substituted heterocyclyl. In certain embodiments, R² is —H, optionally substituted alkyl, optionally substituted heteroalkyl, or optionally substituted heterocyclyl. In certain embodiments, R² is optionally substituted alkyl, optionally substituted heteroalkyl, or optionally substituted heterocyclyl.

In certain embodiments, R² is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R² is —H, optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R² is optionally substituted C₁-C₂₅ alkyl, optionally substituted C₁-C₂₅ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In some embodiments, R² is optionally substituted C₁-C₂₅ alkyl, optionally substituted C₁-C₂₅ heteroalkyl, optionally substituted C₃-C₈ cycloalkyl, or optionally substituted 3- to 8-membered heterocyclyl. In certain embodiments, R² is —H, optionally substituted C₁-C₂₅ alkyl, optionally substituted C₁-C₂₅ heteroalkyl, or optionally substituted 3- to 8-membered heterocyclyl. In certain embodiments, R² is optionally substituted C₁-C₂₅ alkyl, optionally substituted C₁-C₂₅ heteroalkyl, or optionally substituted 3- to 8-membered heterocyclyl.

In certain embodiments, R² is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, optionally substituted 3- to 8-membered heterocyclyl, or a protecting group. In some embodiments, R² is —H, optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In certain embodiments, R² is optionally substituted C₁-C₂₀ alkyl, optionally substituted C₁-C₂₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In some embodiments, R² is optionally substituted C₁-C₂₀ alkyl, optionally substituted C₁-C₂₀ heteroalkyl, optionally substituted C₃-C₈ cycloalkyl, or optionally substituted 3- to 8-membered heterocyclyl. In certain embodiments, R² is —H, optionally substituted C₁-C₂₀ alkyl, optionally substituted C₁-C₂₀ heteroalkyl, or optionally substituted 3- to 8-membered heterocyclyl. In certain embodiments, R² is optionally substituted C₁-C₂₀ alkyl, optionally substituted C₁-C₂₀ heteroalkyl, or optionally substituted 3- to 8-membered heterocyclyl.

In some embodiments, R² is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃₋₈ cycloalkyl, optionally substituted 3- to 8-membered heterocyclyl, or a protecting group. In some embodiments, R² is —H, optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-s cycloalkyl, or optionally substituted 3- to 8-membered heterocyclyl. In certain embodiments, R² is optionally substituted C₁-C₁₀ alkyl, optionally substituted C₁-C₁₀ heteroalkyl, optionally substituted C₆-C₁₄ aryl, optionally substituted 5- to 14-membered heteroaryl, optionally substituted C₃-C₈ cycloalkyl, or optionally substituted 3- to 8-membered heterocyclyl. In some embodiments, R² is —H, optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In some embodiments, R² is optionally substituted C₁-C₁₀ alkyl, optionally substituted C₁-C₁₀ heteroalkyl, optionally substituted C₃-C₈ cycloalkyl, or optionally substituted 3- to 8-membered heterocyclyl. In certain embodiments, R² is —H, optionally substituted C₁-C₁₀ alkyl, optionally substituted C₁-C₁₀ heteroalkyl, or optionally substituted 3- to 8-membered heterocyclyl. In certain embodiments, R² is optionally substituted C₁-C₁₀ alkyl, optionally substituted C₁-C₁₀ heteroalkyl, or optionally substituted 3- to 8-membered heterocyclyl.

In some embodiments, R² is a protecting group. In some embodiments the protecting group is a nitrogen protecting group. In some embodiments, R² is —H. In some embodiments, R² is —H or a protecting group. In some embodiments, R² is —H or a nitrogen protecting group.

In certain embodiments, R² is optionally substituted alkyl. In some embodiments, R² is optionally substituted C₁-C₂₅ alkyl. In certain embodiments, R² is optionally substituted C₁-C₂₀ alkyl. In some embodiments, R² is optionally substituted C₁-C₁₅ alkyl. In certain embodiments, R² is optionally substituted C₁-C₁₀ alkyl. In some embodiments, R² is optionally substituted C₁-C₆ alkyl. In certain embodiments, R² is substituted alkyl. In some embodiments, R² is substituted C₁-C₂₅ alkyl. In certain embodiments, R² is substituted C₁-C₂₀ alkyl. In some embodiments, R² is substituted C₁-C₁₅ alkyl. In certain embodiments, R² is substituted C₁-C₁₀ alkyl. In some embodiments, R² is substituted C₁-C₆ alkyl. In certain embodiments, R² is unsubstituted alkyl. In some embodiments, R² is unsubstituted C₁-C₂₅ alkyl. In certain embodiments, R² is unsubstituted C₁-C₂₀ alkyl. In some embodiments, R² is unsubstituted C₁-C₁₅ alkyl. In certain embodiments, R² is unsubstituted C₁-C₁₀ alkyl. In some embodiments, R² is unsubstituted C₁-C₆ alkyl. In certain embodiments, R² is methyl, ethyl, propyl, or butyl. In some embodiments, R² is methyl or ethyl. In certain embodiments, R² is methyl. In some embodiments, R² is ethyl.

In certain embodiments, R² is optionally substituted heteroalkyl. In some embodiments, R² is optionally substituted C₁-C₂₅ heteroalkyl. In certain embodiments, R² is optionally substituted C₁-C₂₀ heteroalkyl. In some embodiments, R² is optionally substituted C₁-C₁₅ heteroalkyl. In certain embodiments, R² is optionally substituted C₁-C₁₀ heteroalkyl. In some embodiments, R² is optionally substituted C₁-C₆ heteroalkyl. In certain embodiments, R² is substituted heteroalkyl. In some embodiments, R² is substituted C₁-C₂₅ heteroalkyl. In certain embodiments, R² is substituted C₁-C₂₀ heteroalkyl. In some embodiments, R² is substituted C₁-C₁₅ heteroalkyl. In certain embodiments, R² is substituted C₁-C₁₀ heteroalkyl. In some embodiments, R² is substituted C₁-C₆ heteroalkyl. In certain embodiments, R² is unsubstituted heteroalkyl. In some embodiments, R² is unsubstituted C₁-C₂₅ heteroalkyl. In certain embodiments, R² is unsubstituted C₁-C₂₀ heteroalkyl. In some embodiments, R² is unsubstituted C₁-C₁₅ heteroalkyl. In certain embodiments, R² is unsubstituted C₁-C₁₀ heteroalkyl. In some embodiments, R² is unsubstituted C₁-C₆ heteroalkyl.

In certain embodiments, R² is optionally substituted heteroalkyl comprising one or more N atoms. In some embodiments, R² is optionally substituted heteroalkyl, wherein the optionally substituted alkyl comprises two N atoms. In certain embodiments, R² is optionally substituted heteroalkyl, wherein the optionally substituted alkyl comprises three N atoms. In certain embodiments, R² is

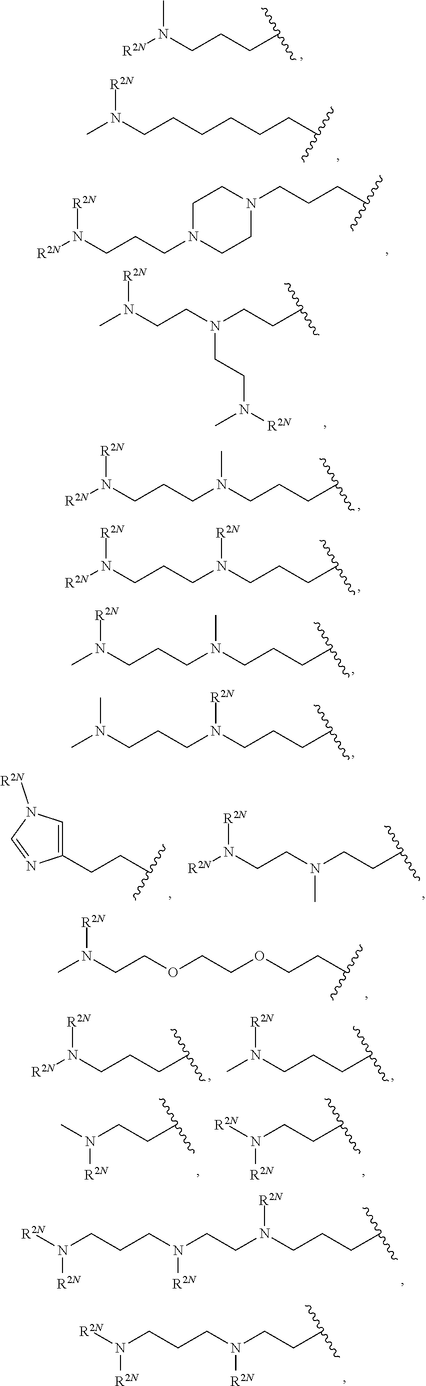

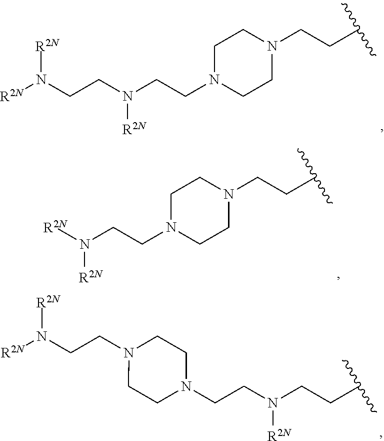

wherein each instance of R^{2N} is —H. In certain embodiments, R² is

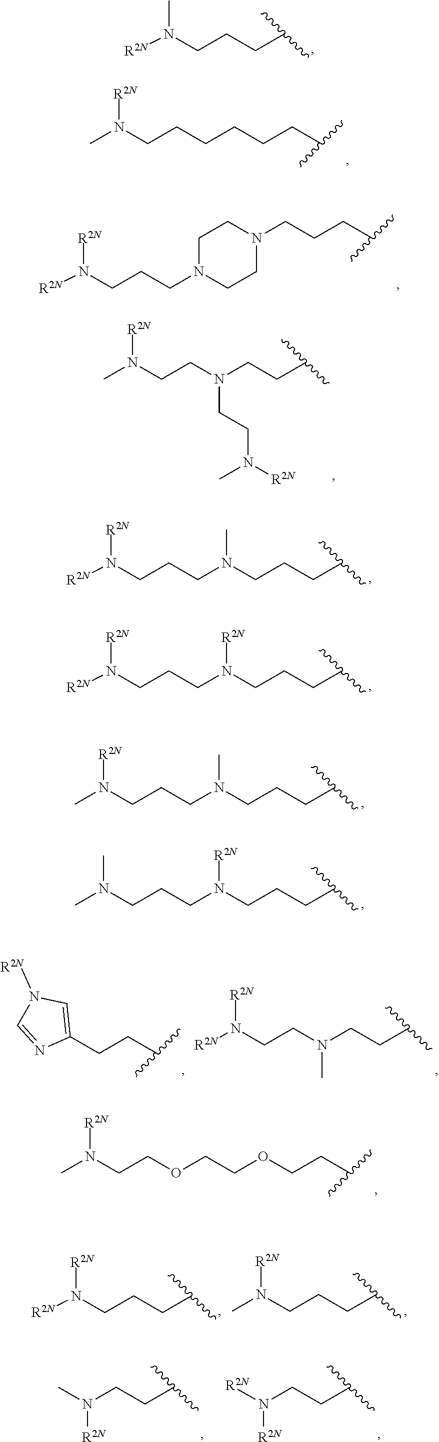

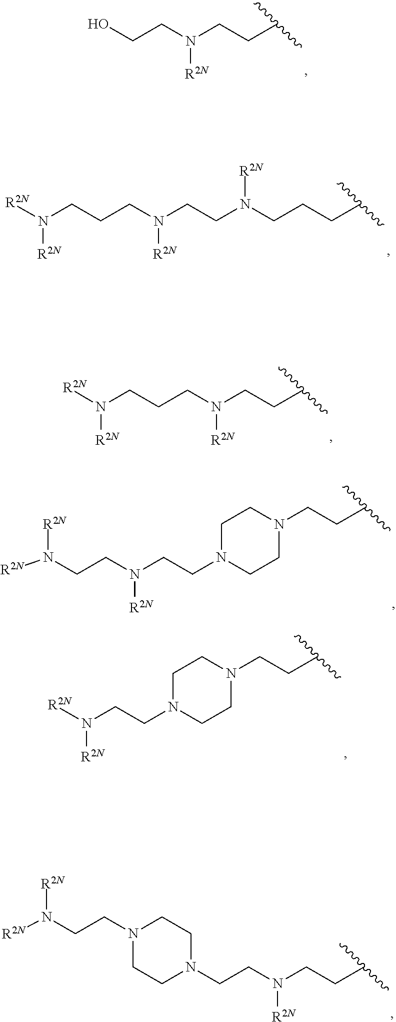

wherein each instance of R^{2N} is independently —H, optionally substituted C₁₋₁₀ aliphatic, optionally substituted C₁₋₁₀ heteroaliphatic, optionally substituted acyl, or a nitrogen protecting group. In certain embodiments, R² is

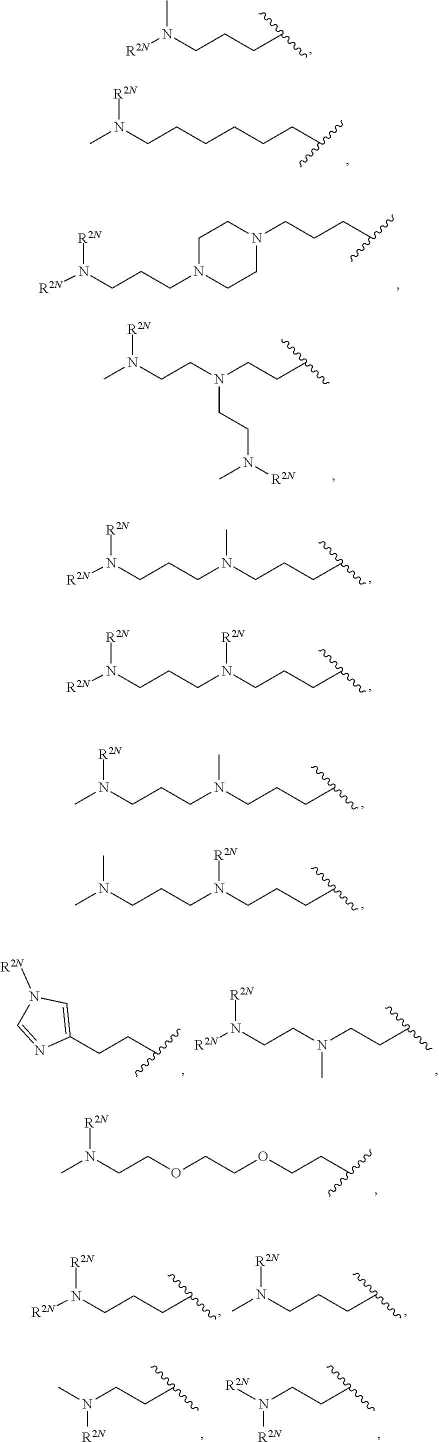

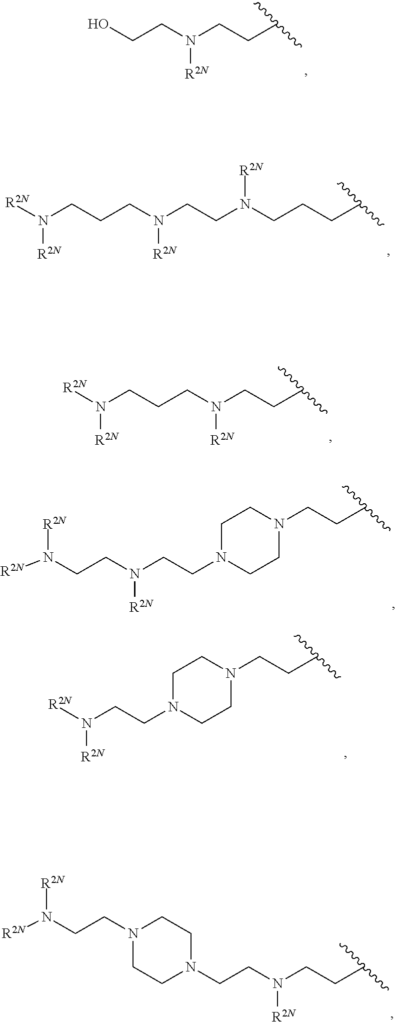

wherein each instance of R^{2N} is independently —H, optionally substituted C₁₋₁₀ aliphatic, optionally substituted C₁₋₁₀ heteroaliphatic, optionally substituted acyl, a nitrogen protecting group, or

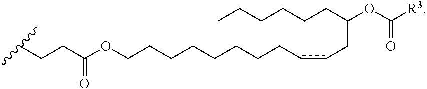

In some embodiments, R^{2N} is —H or

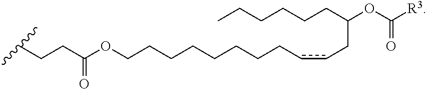

In some embodiments, R^{2N} is —H. In certain embodiments, R^{2N} is

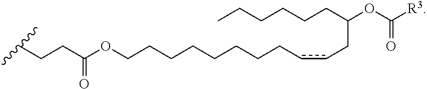

In some embodiments, R^{2N} is optionally substituted acyl. In some embodiments, R^{2N} is a nitrogen protecting group. In some embodiments, R^{2N} is Bn, Boc, Cbz, Fmoc, trifluoroacetyl, triphenylmethyl, acetyl, or Ts. In some embodiments, R^{2N} is optionally substituted C₁₋₁₀ aliphatic or optionally substituted C₁₋₁₀ heteroaliphatic. In some embodiments, R^{2N} is optionally substituted C₁₋₁₀ aliphatic. In some embodiments, R^{2N} is C₁₋₁₀ alkyl. In some embodiments, R^{2N} is -Me, -Et, -nPr, -iPr, -nBu, -iBu, or -tBu. In some embodiments, R^{2N} is -Me or -Et. In some embodiments, R^{2N} is -Me. In some embodiments, R^{2N} is -Et. In some embodiments, R^{2N} is C₂₋₁₀ alkenyl. In some embodiments, R^{2N} is C₂₋₁₀ alkynyl. In some embodiments, R^{2N} is optionally substituted C₁₋₁₀ heteroaliphatic. In some embodiments, R^{2N} is C₁₋₁₀ heteroalkyl. In some embodiments, R^{2N} is C₂₋₁₀ heteroalkenyl. In some embodiments, R^{2N} is C₂₋₁₀ heteroalkynyl.

In some embodiments, R² is optionally substituted C₆-C₁₄ aryl. In some embodiments, R² is substituted C₆-C₁₄ aryl. In some embodiments, R² is unsubstituted C₆-C₁₄ aryl. In certain embodiments, R² is optionally substituted C₆-C₁₀ aryl. In certain embodiments, R² is substituted C₆-C₁₀ aryl. In certain embodiments, R² is unsubstituted C₆-C₁₀ aryl. In some embodiments, R² is optionally substituted phenyl or optionally substituted naphthyl. In certain embodiments, R² is optionally substituted phenyl. In some embodiments, R² is optionally substituted naphthyl.

In some embodiments, R² is optionally substituted 5- to 14-membered heteroaryl. In some embodiments, R² is substituted 5- to 14-membered heteroaryl. In some embodiments, R² is unsubstituted 5- to 14-membered heteroaryl. In some embodiments, R² is optionally substituted 5- to 10-membered heteroaryl. In some embodiments, R² is substituted 5- to 10-membered heteroaryl. In some embodiments, R² is unsubstituted 5- to 10-membered heteroaryl. In some embodiments, R² is optionally substituted pyridinyl.

In some embodiments, R² is optionally substituted C₃-C₈ cycloalkyl. In some embodiments, R² is substituted C₃-C₈ cycloalkyl. In some embodiments, R² is unsubstituted C₃-C₈ cycloalkyl. In some embodiments, R² is C₃-C₈ cycloalkyl substituted with optionally substituted alkyl, optionally substituted heteroalkyl, optionally substituted cycloalkyl, optionally substituted heterocyclyl, optionally substituted heteroaryl, or optionally substituted aryl.

In some embodiments, R² is optionally substituted 3- to 8-membered heterocyclyl. In some embodiments, R² is substituted 3- to 8-membered heterocyclyl. In some embodiments, R² is unsubstituted 3- to 8-membered heterocyclyl. In some embodiments, R² is 3- to 8-membered heterocyclyl substituted with optionally substituted alkyl, optionally substituted heteroalkyl, optionally substituted cycloalkyl, optionally substituted heterocyclyl, optionally substituted heteroaryl, or optionally substituted aryl.

In certain embodiments, R² is selected from the group consisting of -Me,

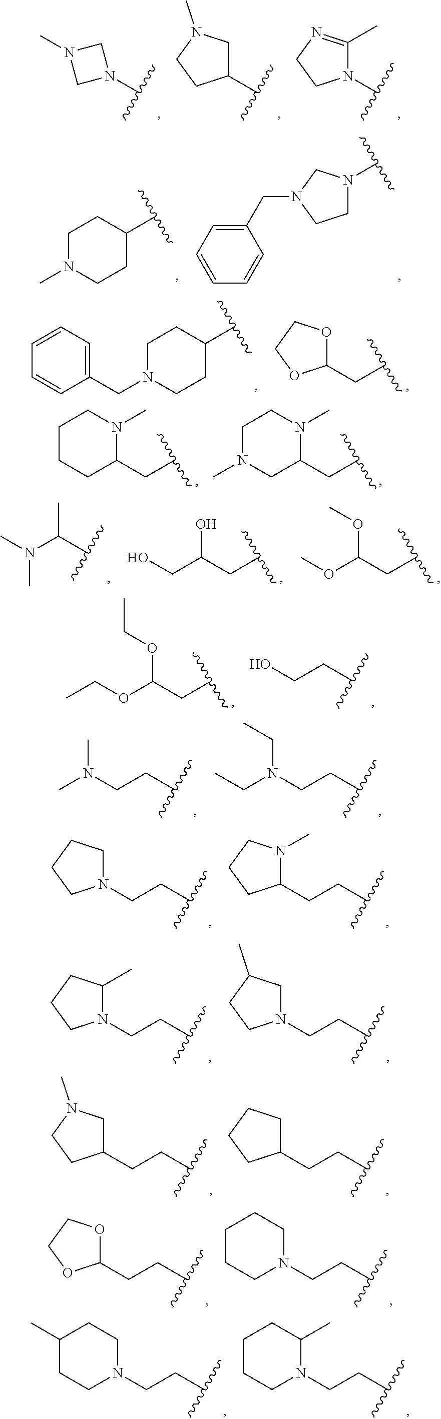

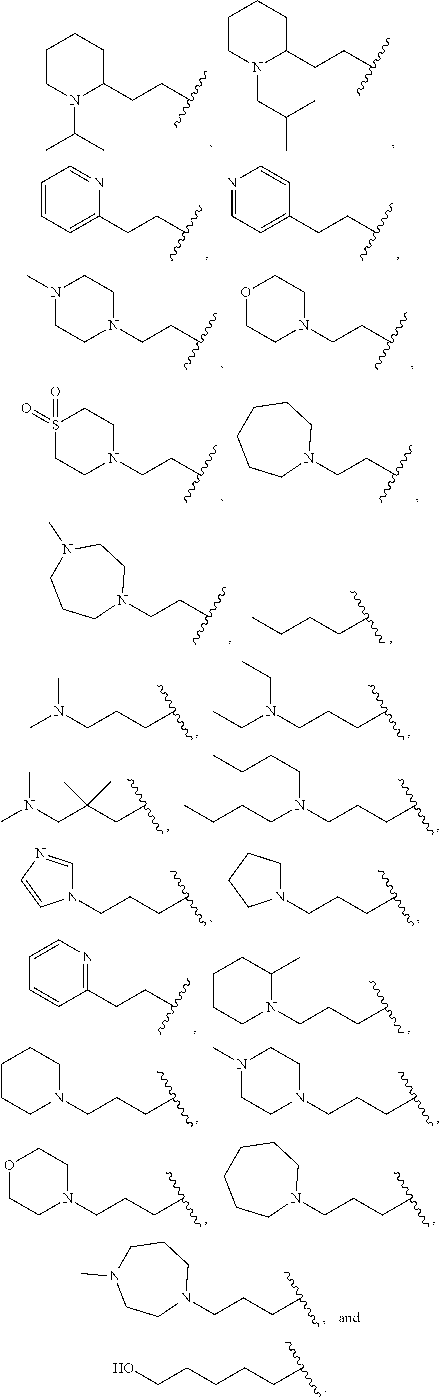

In certain embodiments, R² is selected from the group consisting of -Me,

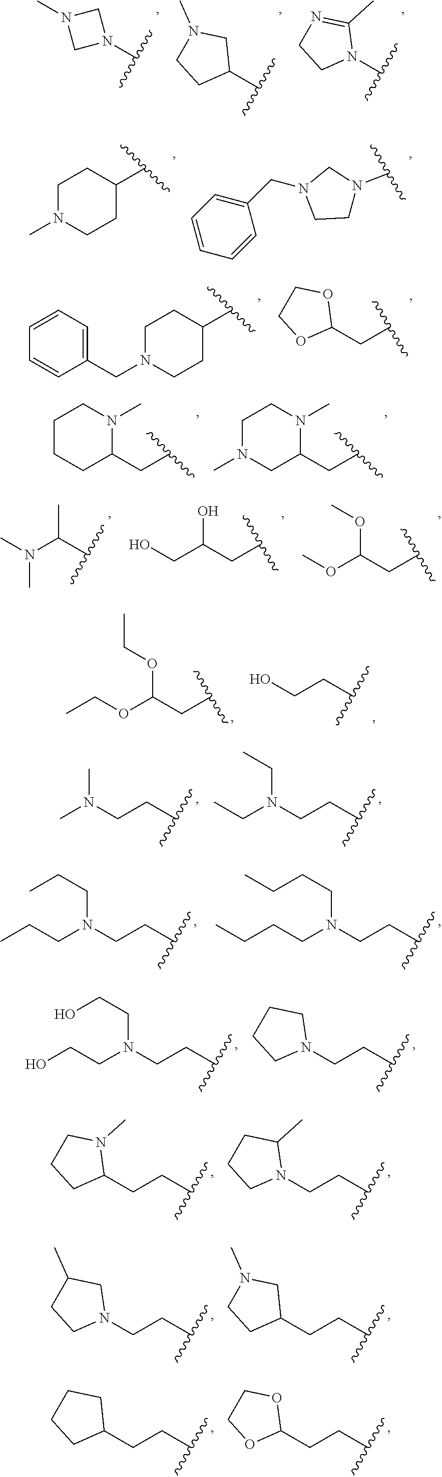

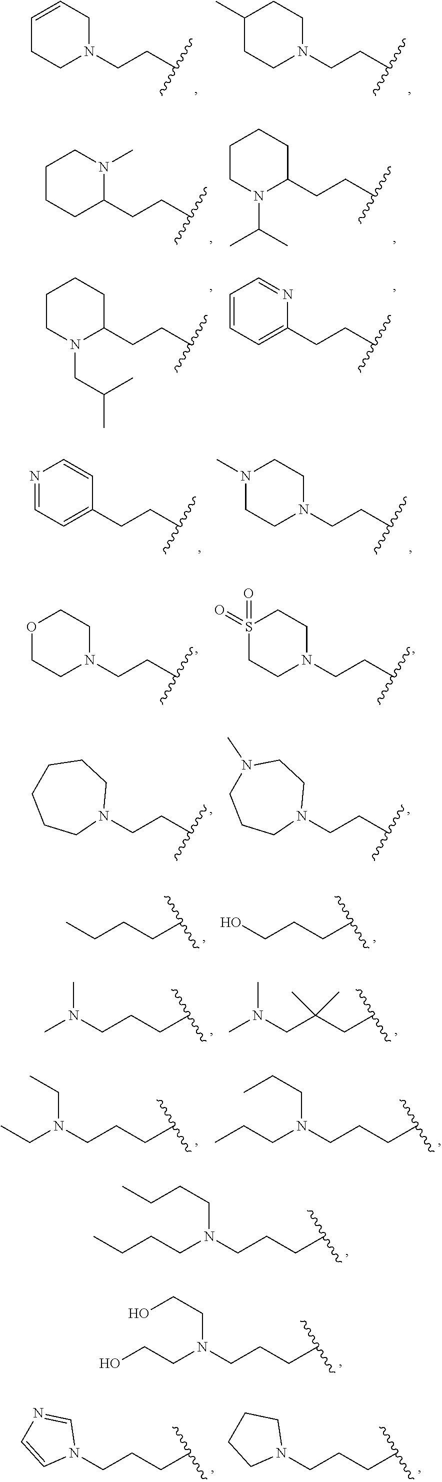

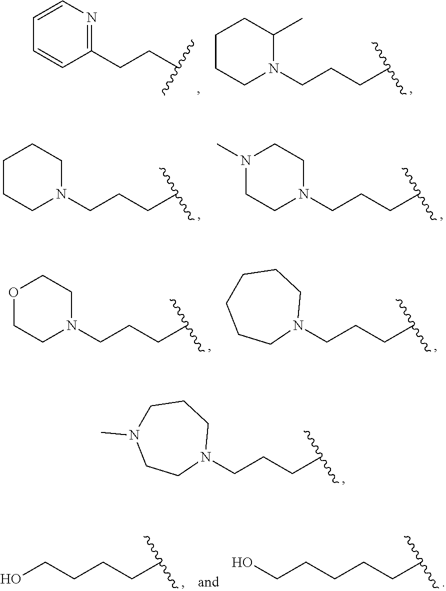

In some embodiments, R¹ is not —H. In certain embodiments, R² is not —H. In some embodiments, R¹ and R² are not both H.

In certain embodiments, R¹ and R² are joined together with the intervening atoms to form an optionally substituted heterocycle. In some embodiments, R¹ and R² are joined together with the intervening atoms to form an optionally substituted N-heterocycle. In certain embodiments, R¹ and R² are joined together with the intervening atoms to form an optionally substituted 3- to 6-membered heterocycle. In some embodiments, R¹ and R² are joined together with the intervening atoms to form an optionally substituted 3- to 6-membered N-heterocycle. In some embodiments, R¹ and R² are joined together with the intervening atoms to form a substituted 3- to 6-membered N-heterocycle. In some embodiments, R¹ and R² are joined together with the intervening atoms to form an unsubstituted 3- to 6-membered N-heterocycle. In certain embodiments, R¹ and R² are joined together with the intervening atoms to form optionally substituted aziridine, azetidine, pyrrolidine, piperidine, or piperazine. In certain embodiments, R¹ and R² are joined together with the intervening atoms to form aziridine, azetidine, pyrrolidine, piperidine, or piperazine substituted with C₁-C₁₀ alkyl, C₁-C₁₀ heteroalkyl, C₃-C₈ carbocyclyl, 3- to 8-membered heterocyclyl, C₆-C₁₄ aryl, or 5- to 14-membered heteroaryl. In certain embodiments, R¹ and R² are joined together with the intervening atoms to form unsubstituted aziridine, azetidine, pyrrolidine, piperidine, or piperazine.

In certain embodiments, X is —NR¹R² wherein R¹ and R² are joined together with the intervening atoms to form an optionally substituted heterocycle selected from the group consisting of

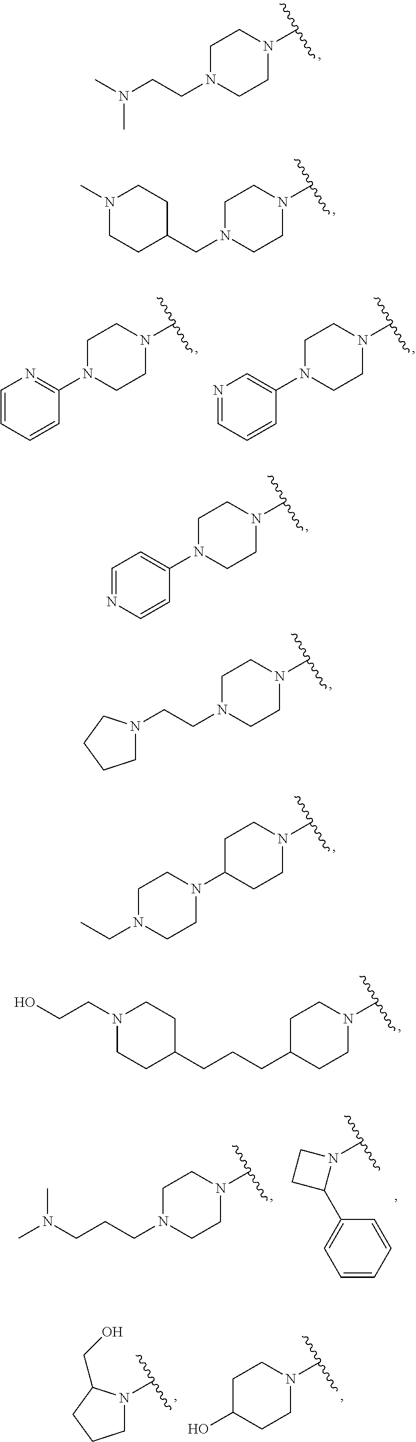

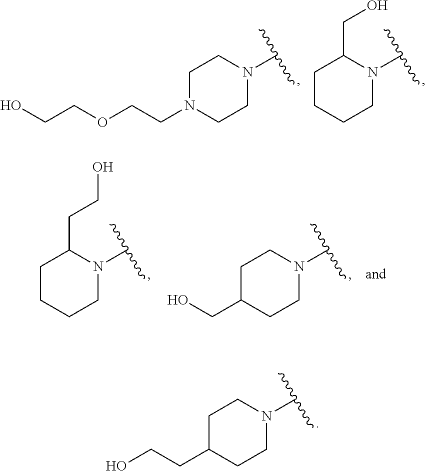

In certain embodiments, X is —NR¹R² wherein R¹ and R² are joined together with the intervening atoms to form an optionally substituted heterocycle selected from the group consisting of

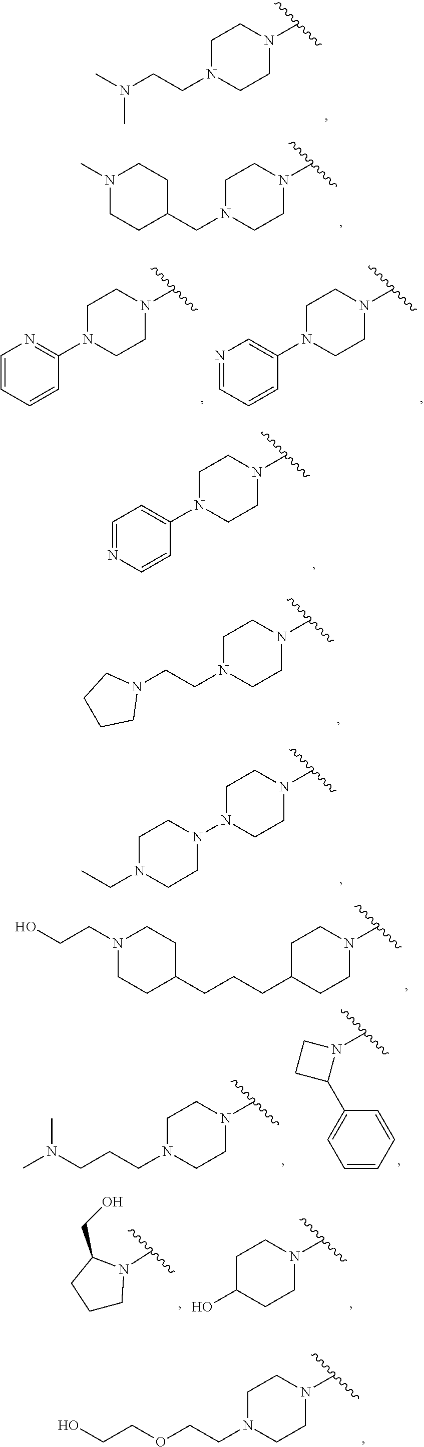

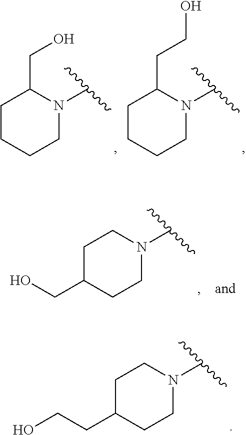

In some embodiments, R¹ and R² are joined together with the intervening atoms to form an optionally substituted heterocycle comprising one or more N atoms. In certain embodiments, R¹ and R² are joined together with the intervening atoms to form an optionally substituted heterocycle comprising two N atoms. In some embodiments, R¹ and R² are joined together with the intervening atoms to form an optionally substituted heterocycle comprising three N atoms. In some embodiments, R¹ and R² are joined together with the intervening atoms to form an optionally substituted heterocycle selected from the group consisting of:

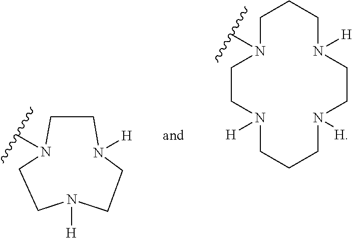

In certain embodiments,

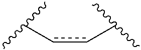

represents a single bond. In some embodiments,

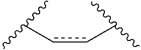

represents a double bond.

Also provided herein is a method of preparing a dry powder composition, the method comprising: a) providing an aqueous mixture comprising a polynucleotide, a lipid nanoparticle, and a saccharide excipient, wherein the polynucleotide is encapsulated by the lipid nanoparticle; and b) spray freeze drying the mixture to form microparticles, wherein at least about 50% of the microparticles in the composition have an aerodynamic size of about 1 micron to about 5 microns, a physical diameter of about 3 microns to about 10 microns, or a combination thereof, thereby preparing the dry powder composition.

In some embodiments, at least about 70% of the microparticles in the composition have an aerodynamic size of about 1 micron to about 5 microns. In certain embodiments, at least about 70% of the microparticles in the composition have a physical diameter of between about 3 microns and about 10 microns.

In some embodiments, the polynucleotide is at least about 30 nucleotides in length. In certain embodiments, the polynucleotide is at least about 1,000 nucleotides in length. In some embodiments, the polynucleotide is a messenger RNA (mRNA). In certain embodiments, the mRNA encodes an antigen. In some embodiments, the polynucleotide is any polynucleotide provided herein.

In some embodiments, the aqueous mixture comprises about 10 μg/ml to about 50 μg/ml of the polynucleotide. In certain embodiments, the aqueous mixture comprises about 20 μg/ml to about 30 μg/ml of the polynucleotide.

In some embodiments, the aqueous mixture comprises about 0.2 mg/ml to about 0.6 mg/ml total lipid mass. In certain embodiments, the aqueous mixture comprises about 0.4 mg/mL to about 0.5 mg/ml total lipid mass.

In certain embodiments, the saccharide excipient comprises a polyol. In some embodiments, the polyol is mannitol or erythritol. In some embodiments, the polyol is mannitol. In certain embodiments, the polyol is erythritol. In some embodiments, the saccharide comprises an oligosaccharide. In some embodiments, the oligosaccharide is trehalose or lactose. In some embodiments, the oligosaccharide is trehalose. In some embodiments, the oligosaccharide is lactose. In some embodiments, the saccharide excipient is mannitol, erythritol, trehalose, or lactose. In some embodiments, the saccharide excipient is mannitol, erythritol, or trehalose. In some embodiments, the saccharide excipient is mannitol. In some embodiments, the saccharide excipient is erythritol. In some embodiments, the saccharide excipient is trehalose. In some embodiments, the saccharide excipient is lactose.

In some embodiments, the lipid nanoparticle comprises about 2% to about 20% of the saccharide excipient. In certain embodiments, the lipid nanoparticle comprises about 2% to about 4% of the saccharide excipient.

In some embodiments, the aqueous mixture comprises about 0.25% to about 2.5% of the saccharide excipient. In certain embodiments, the aqueous mixture comprises about 0.25% to about 0.5% of the saccharide excipient.

In some embodiments, the weight ratio of the saccharide excipient to the polynucleotide is about 50:1 to about 1000:1. In certain embodiments, the weight ratio of the saccharide excipient to polynucleotide is about 50:1 to about 200:1. In some embodiments, the weight ratio of the saccharide excipient to polynucleotide is about 100:1.

In some embodiments, the lipid nanoparticle comprises an ionizable lipid and one or more of a charged lipid and a phospholipid.

In some embodiments, the ionizable lipid is CKK-E12 (MD-1), C12-200, Dlin-MC3-DMA (MC3), a compound of Formula (I), a compound of Formula (II), or a pharmaceutically acceptable salt, solvate, tautomer, stereoisomer, or isotopically labeled derivative thereof, or a lipid molecule that is neutral at physiological pH, but will be protonated at lower pH, which makes it positively charged. In certain embodiments, the ionizable lipid is CKK-E12 (MD-1), C12-200, Dlin-MC3-DMA (MC3), RCB-01-223-3, RCB-02-76-3, or a pharmaceutically acceptable salt, solvate, tautomer, stereoisomer, or isotopically labeled derivative thereof or a lipid molecule that is neutral at physiological pH, but will be protonated at lower pH, which makes it positively charged. In some embodiments, the ionizable lipid is CKK-E12 (MD-1), C12-200, Dlin-MC3-DMA (MC3), a compound of Formula (I), or a pharmaceutically acceptable salt, solvate, tautomer, stereoisomer, or isotopically labeled derivative thereof, or a lipid molecule that is neutral at physiological pH, but will be protonated at lower pH, which makes it positively charged. In some embodiments, the ionizable lipid is CKK-E12 (MD-1), C12-200, Dlin-MC3-DMA (MC3), a compound of Formula (II), or a pharmaceutically acceptable salt, solvate, tautomer, stereoisomer, or isotopically labeled derivative thereof, or a lipid molecule that is neutral at physiological pH, but will be protonated at lower pH, which makes it positively charged.

In some embodiments, the ionizable lipid is CKK-E12 (MD-1), or a pharmaceutically acceptable salt, solvate, tautomer, stereoisomer, or isotopically labeled derivative thereof. In some embodiments, the ionizable lipid is CKK-E12 (MD-1). In certain embodiments, the ionizable lipid is C12-200, or a pharmaceutically acceptable salt, solvate, tautomer, stereoisomer, or isotopically labeled derivative thereof. In certain embodiments, the ionizable lipid is C12-200. In some embodiments, the ionizable lipid is Dlin-MC3-DMA (MC3), or a pharmaceutically acceptable salt, solvate, tautomer, stereoisomer, or isotopically labeled derivative thereof. In some embodiments, the ionizable lipid is Dlin-MC3-DMA (MC3). In some embodiments, the ionizable lipid is a compound provided herein (e.g., a compound of Formula (I) or (II)), or a pharmaceutically acceptable salt, solvate, tautomer, stereoisomer, or isotopically labeled derivative thereof. In some embodiments, the ionizable lipid is a compound of Formula (I), or a pharmaceutically acceptable salt, solvate, tautomer, stereoisomer, or isotopically labeled derivative thereof. In some embodiments, the ionizable lipid is a compound of Formula (II), or a pharmaceutically acceptable salt, solvate, tautomer, stereoisomer, or isotopically labeled derivative thereof. In some embodiments, the ionizable lipid is RCB-01-223-3 or RCB-02-76-3, or a pharmaceutically acceptable salt, solvate, tautomer, stereoisomer, or isotopically labeled derivative thereof. In some embodiments, the ionizable lipid is RCB-01-223-3, or a pharmaceutically acceptable salt, solvate, tautomer, stereoisomer, or isotopically labeled derivative thereof. In some embodiments, the ionizable lipid is RCB-01-223-3. In some embodiments, the ionizable lipid is RCB-02-76-3, or a pharmaceutically acceptable salt, solvate, tautomer, stereoisomer, or isotopically labeled derivative thereof. In certain embodiments, the ionizable lipid is RCB-02-76-3. In some embodiments, the ionizable lipid is a lipid molecule that is neutral at physiological pH, but will be protonated at lower pH, which makes it positively charged.

In certain embodiments, the charged lipid is any charged lipid provided herein. In some embodiments, the charged lipid is 1,2-dioleoyl-3-trimethylammonium-propane (DOTAP), dimethyldioctadecylammonium (DDA), or a combination thereof. In certain embodiments, the charged lipid is DOTAP. In some embodiments, the charged lipid is DDA.In some embodiments, the charged lipid is a cationic lipid. In some embodiments, the cationic lipid is 1,2-dioleoyl-3-trimethylammonium-propane (DOTAP), dimethyldioctadecylammonium (DDA), or a combination thereof. In certain embodiments, the cationic lipid is DOTAP. In some embodiments, the cationic lipid is DDA.

In certain embodiments, the phospholipid is any phospholipid provided herein. In some embodiments, the phospholipid is 1,2-dioleoyl-sn-glycero-3-phosphoethanolamine (DOPE) or 1,2-distearoyl-sn-glycero-3-phosphocholine (DSPC), or a combination thereof. In certain embodiments, the phospholipid is DOPE. In some embodiments, the phospholipid is DSPC.

In some embodiments, the lipid nanoparticle further comprises a lipid conjugated to a solubilizing group. In certain embodiments, the solubilizing group is a polymer of polyethylene glycol (PEG). In some embodiments, the lipid nanoparticle further comprises any PEG-lipid provided herein.

In some embodiments, the lipid nanoparticle further comprises any sterol provided herein. In certain embodiments, the lipid nanoparticle further comprises a substituted or unsubstituted cholesterol. In certain embodiments, the lipid nanoparticle further comprises a substituted cholesterol. In certain embodiments, the lipid nanoparticle further comprises an unsubstituted cholesterol.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1

Common Precursor ricinoleic acrylate 12-hydroxy stearyl acrylate

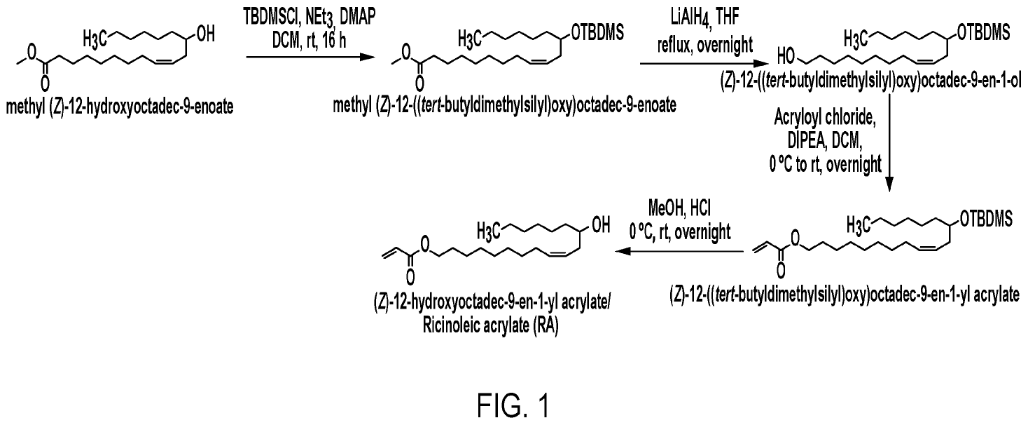

The common precursors ricinoleic acrylate or 12-hydroxy stearyl acrylate were synthesized by using a simple four step procedure and from very inexpensive materials (FIG. 1). In the first step, the secondary alcohol was protected with TBDMS followed by LiAlH4 reduction of ester to yield a primary alcohol. Addition of acryloyl chloride onto the primary alcohol synthesized the acrylate, followed by deprotection of alcohol, yielded the common precursor.

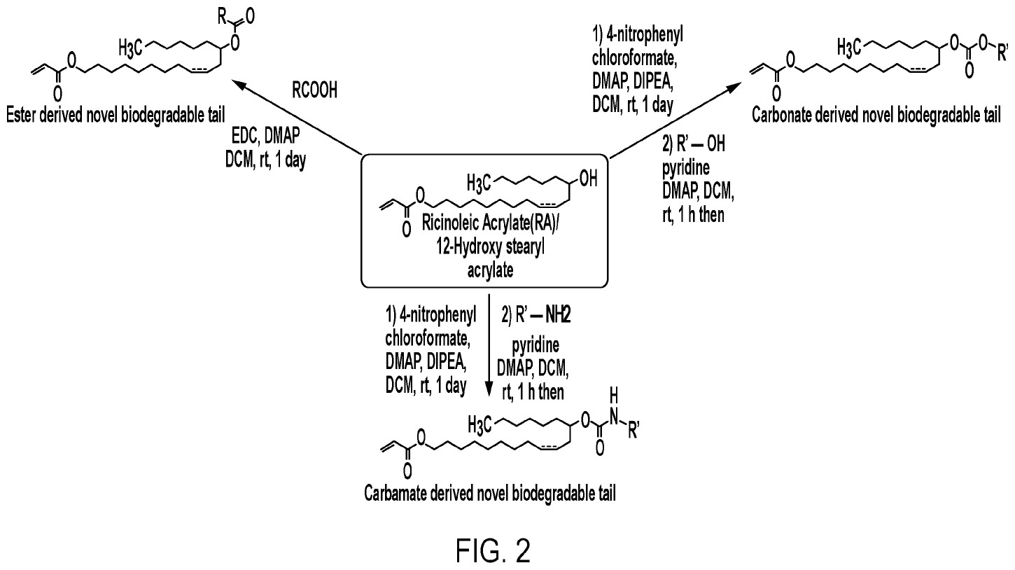

The common precursor can be further transformed to either esters, carbonates, or carbamates upon reacting with acids, alcohols, and amines, respectively (FIG. 2). These esters, carbonates, and carbamates can react with primary or secondary amines to yield lipids.

By using the Michael addition reaction strategy, reaction with a primary amine or a secondary amine formed lipids with two tails or one tail, respectively (FIG. 3). Around 150 ester-derived lipids were synthesized and characterized. For carbonate-derived lipids, around 50 lipids were tested. All these compounds were verified by NMR and IRMS to have greater than 95% purity. pKa studies for the lipid with encapsulated mRNA demonstrated pKas in the range of 5.8 to 7.2, which is appropriate for endosomal escape.⁶

### Synthetic Procedures

Unless otherwise noted, all manipulations were carried out under an Argon atmosphere using standard Schlenk-line techniques. All glassware were oven-dried at 150° C. for overnight prior to use. Anhydrous THF, DCM, DMF, toluene, and ether were purchased from Sigma Aldrich as sure-seal bottles and used as received under Argon atmosphere. Amines were purchased from Sigma-Aldrich, TCI America, Alfa Aesar, Ambeed, and Enamine. All other starting materials were obtained from commercial sources or outsourced and were used as received. High throughput reactions were carried out in 96-well deep-well plates with glass inserts (VWR catalog #97055-472).

¹H NMR spectra were obtained on a 500 MHz spectrometer, and chemical shifts were recorded relative to residual protio solvent peak (CDCl₃: δ 7.26, CD3OD: δ 3.31). Data are reported as follows: chemical shift, multiplicity (s=singlet, d=doublet, dd=doublet of doublet, t=triplet, q=quartet, m=multiplet), integration and coupling constants are reported in Hz. ¹³C NMR spectra were obtained on a 125 MHz spectrometer and chemical shifts were recorded to the solvent resonance with carbon and proton decoupling. Both ¹H and ¹³C NMR chemical shifts were reported in parts per million downfield from tetramethylsilane (δ=0). High-resolution mass spectra were obtained on a BioTOf IRMS at DCIF facility (MIT Chemistry).

### Synthesis of methyl (Z)-12-((tert-butyldimethylsilyl)oxy)octadic-9-enoate

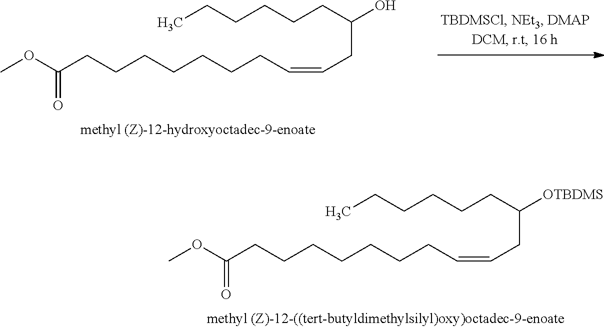 [SMILES: CCCCCCC(O)C/C=C\CCCCCCCC(=O)OC ; CCCCCCC(C/C=C\CCCCCCCC(=O)OC)O[Si](C)(C)C(C)(C)C]

To an oven dried 250-mL round-bottom flask equipped with a magnetic stir bar, methyl (Z)-12-hydroxyoctadec-9-enoate (methyl Ricinoleate, CAS #141-24-2)) (10.0 g, 32.0 mmol) was added and then suspended in 80 mL of DMF. Imidazole (4.4 g, 64.0 mmol) was then added as a solid and allowed to stir for 15 min. Tert-butyldimethylsilyl chloride (7.3 mL, 48.0 mmol) was then added as a solid and allowed to stir at room temperature for overnight. The reaction was quenched with 0.1M HCl and extracted with three times with DCM (3×100 mL) and dried over anhydrous MgSO₄. Solvents were removed in vacuo. Products were purified via SiO₂ gel flash column chromatography using hexanes and ethyl acetate as eluent at 50:1 to afford methyl (Z)-12-(tert-butyldimethylsilyl)oxy)octadic-9-enoate as a colorless liquid; Yield: 12.3 g, 90%.

¹H-NMR (500 MHz, CDCl₃): δ 5.41-5.43 (m, 2H), 4.12 (q, J=7.0 Hz, 1H), 3.68 (s, 3H), 2.30 (t, J=7.5 Hz, 2H), 2.19 (t, J=5.5 Hz, 2H), 2.01-2.06 (m, 3H), 1.61-1.67 (m, 2H), 1.26-1.42 (m, 19H), 0.90 (s, 9H), −0.06 (s, 6H).

¹³C-NMR (125 MHz, CDCl₃): δ 174.3, 131.3, 126.0, 72.4, 60.4, 51.4, 36.9, 35.3, 34.1, 31.9, 29.6, 29.5, 29.18, 29.16, 29.1, 27.4, 25.9, 25.4, 25.9, 22.6, 21.1, 18.2, 14.2, 14.1, 4.3.

### Synthesis of (Z)-12-((tert-butyldimethylsilyl)oxy)octadic-9-en-1-ol

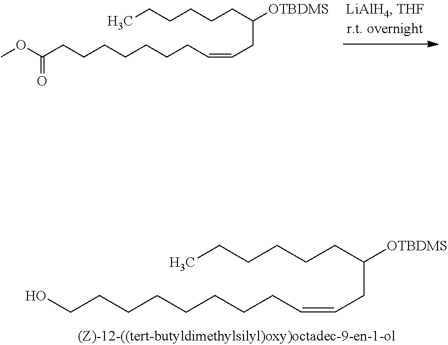 [SMILES: CCCCCCC(C/C=C\CCCCCCCC(=O)OC)O[Si](C)(C)C(C)(C)C ; CCCCCCC(C/C=C\CCCCCCCCO)O[Si](C)(C)C(C)(C)C]

To an oven dried two neck 250 mL round bottom flask containing LiAlH4 (1.31 g, 34.6 mmol) was slowly added 60 mL THF at 0° C. Methyl (Z)-12-((tert-butyldimethylsilyl)oxy)octadic-9-enoate (12.3 g, 28.8 mmol, 1.0 equiv) was dissolved in 40 mL THF, added slowly over 30 min to the above solution, and stirred at room temperature for overnight. On the next day the reaction was cooled to 0° C. and 10% aq. NaOH solution (10 mL) was added dropwise and stirred for 1 h at room temperature (Fieser workup). The crude reaction was filtered using celite, and the filtrate was dried with anhydrous MgSO₄.

Solvents were removed in vacuo. Products were purified via SiO2 gel flash column chromatography using hexanes and Ethyl acetate as eluent at 25:1 to afford (Z)-12-(((tert-butyldimethylsilyl)oxy)octadic-9-en-1-ol as a colorless liquid; Yield: 8.2 g, 71%.

¹H-NMR (500 MHz, CD3OD): δ 5.41-5.47 (m, 2H), 3.70-3.75 (m, 1H), 3.54 (t, J=7.0 Hz, 2H), 3.32-3.36 (m, 4H), 2.18-2.25 (m, 2H), 2.05-2.08 (m, 2H), 1.51-1.58 (m, 2H), 1.35-1.42 (m, 18H), 0.92 (s, 9H), −0.09 (s, 6H).

¹³C-NMR (125 MHz, CD3OD): δ 131.1, 125.6, 72.3, 61.6, 36.6, 34.8, 32.3, 31.7, 29.4, 29.3, 29.21, 29.16, 29.0, 27.1, 25.6, 25.1, 25.0, 22.3, 17.6, 13.0, −5.5.

### Synthesis of (Z)-12-((tert-butyldimethylsilyl)oxy)octadic-9-en-1-yl acrylate

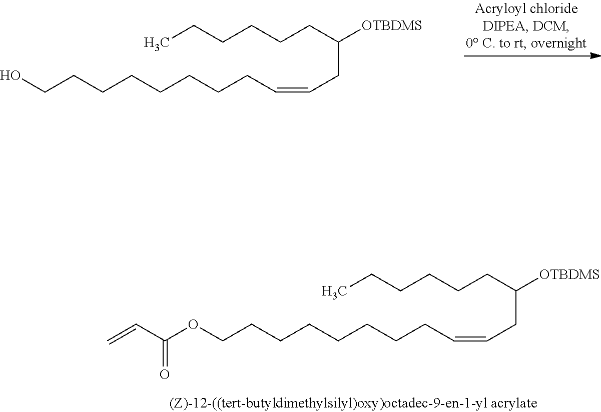 [SMILES: CCCCCCC(C/C=C\CCCCCCCCO)O[Si](C)(C)C(C)(C)C ; C=CC(=O)OCCCCCCCC/C=C\CC(CCCCCC)O[Si](C)(C)C(C)(C)C]

To an oven dried two neck 250 mL round bottom flask was added (Z)-12-(((tert-butyldimethylsilyl)oxy)octadic-9-en-1-ol (8.2 g, 20.6 mmol) followed by 40 mL DCM. the above solution was cooled to 0° C. added DIPEA (9.0 mL, 51.5 mmol) slowly and acryloyl chloride (2.2 mL, 25.0 mmol) was added drop wise over 10 min. The reaction was stirred at room temperature overnight. The crude reaction was quenched with sat. NaHCO₃ and extracted with DCM for 3 times (3×100 mL). The organic layer was dried over anhydrous MgSO₄ and concentrated in vacuo. Crude products were purified via SiO2 gel flash column chromatography using hexanes and ethyl acetate as eluent at 30:1 to afford (Z)-12-((tert-butyldimethylsilyl)oxy)octadic-9-en-1-yl acrylate as a colorless liquid; Yield: 7.2 g, 77%.

¹H-NMR (500 MHz, CDCl₃): δ 6.40 (dd, J1=17.0 Hz, J2=1.5 Hz, 1H), 6.12 (dd, J1=17.0 Hz, J2=10.5 Hz, 1H), 5.82 (dd, J1=10.5 Hz, J2=1.5 Hz, 1H), 5.38-5.47 (m, 2H), 4.16 (t, J=7.0 Hz, 2H), 3.64-3.69 (m, 1H), 2.19 (t, J=5.5 Hz, 2H), 2.02-2.06 (m, 2H), 1.63-1.75 (m, 4H), 1.51-1.55 (m, 2H), 1.29-1.32 (m, 18H), 0.98 (d, J=9.0 Hz, 2H), 0.89-0.91 (m, 9H), 0.07 (s, 6H).

¹³C-NMR (125 MHz, CDCl₃): δ 166.4, 131.4, 130.4, 128.7, 125.9, 72.4, 64.7, 36.9, 35.3, 34.7, 31.9, 31.6, 29.7, 29.5, 29.4, 29.27, 29.25, 28.6, 27.5, 25.9, 25.4, 22.66, 22.65, 14.12, 14.10, −4.34.

### Synthesis of (Z)-12-hydroxyoctadec-9-en-1-yl acrylate/Ricinoleic acrylate (RA)

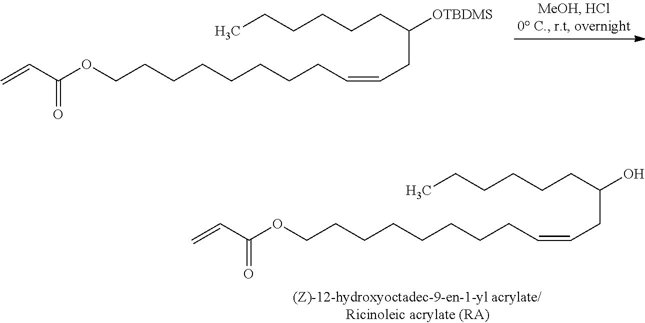 [SMILES: C=CC(=O)OCCCCCCCC/C=C\CC(CCCCCC)O[Si](C)(C)C(C)(C)C ; C=CC(=O)OCCCCCCCC/C=C\CC(O)CCCCCC]

To an oven dried 250-mL round-bottom flask equipped with a magnetic stir bar was added (Z)-12-((tert-butyldimethylsilyl)oxy)octadic-9-en-1-yl acrylate (7.2 g, 16.0 mmol), which was then suspended in 60 mL of MeOH. 1% HCl was then added and allowed to stir overnight at room temperature. The solvent was evaporated under reduced pressure and diluted with water and extracted with ethyl acetate three times (3×100 mL). The organic layer was dried over anhydrous MgSO₄ and concentrated in vacuo. Crude products were purified via SiO₂ gel flash column chromatography using hexanes and ethyl acetate as eluent at 40:1 to afford (Z)-12-hydroxyoctadec-9-en-1-yl acrylate/Ricinoleic acrylate (RA) as a colorless liquid; Yield: 4.3 g, 79%.

¹H-NMR (500 MHz, CDCl₃): δ 6.39 (dd J1=17.5 Hz, J2=1.5 Hz, 1H), 6.11 (dd, J1=17.0 Hz, J2=1.5 Hz, 1H), 5.82 (dd, J1=10.5 Hz, J2=1.5 Hz, 1H), 5.55-5.60 (m, 1H), 5.40-5.45 (m, 1H), 4.15 (t, J=6.5 Hz, 2H), 3.61-3.66 (m, 1H), 2.22 (t, J=7.5 Hz, 2H), 2.05-2.09 (m, 2H), 1.65-1.71 (m, 2H), 1.45-1.51 (m, 2H), 1.26-1.39 (m, 18H), 0.89 (t, J=7.0 Hz, 3H).

¹³C-NMR (125 MHz, CDCl₃): δ 166.4, 133.4, 130.4, 128.7, 125.2, 71.5, 64.7, 38.9, 35.4, 31.9, 29.6, 29.38, 29.36, 29.21, 29.20, 28.6, 27.4, 25.9, 25.7, 22.6, 14.1.

### General Procedure for Esterification of Ricinoleic Acrylate

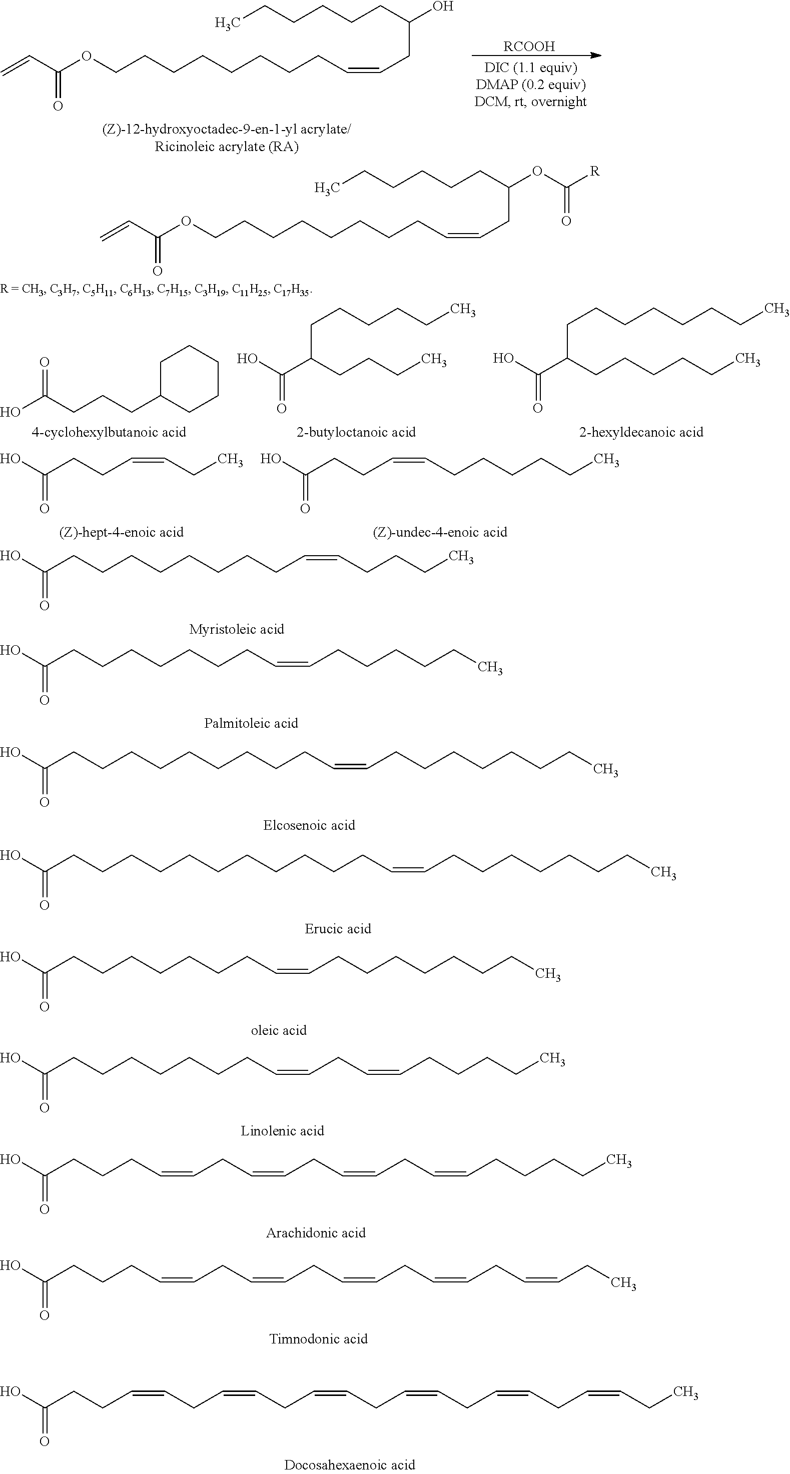

To an oven dried two neck 50 mL round bottom flask under argon atmosphere were added aliphatic carboxylic acid (4.5 mmol, 1.5 equiv), N,N′-Diisopropylcarbodiimide (DIC) (4.5 mmol, 1.5 equiv) and DMAP (0.6 mmol, 0.2 equiv) with 10 mL anhydrous DCM. the reaction mixture was stirred at room temperature. After 30 min Ricinoleic acrylate (RA) (1.0 g, 3.0 mmol, 1.0 equiv, in 5 mL DCM) was added slowly to the above reaction mixture over 10 min and stirred at room temperature overnight. The crude reaction was quenched with sat. NaHCO₃ and extracted with DCM 3 times (3×100 mL). The combined organic layers were washed with brine, dried over anhydrous MgSO₄, and concentrated in vacuo. Crude products were purified via SiO₂ gel flash column chromatography using hexanes and ethyl acetate as eluent at 30:1 to afford esterified Ricinoleic acrylate.

### Synthesis of (Z)-18-(acryloyloxy)octadec-9-en-7-yl (5Z,8Z,11Z,14Z,17Z)-icosa-5,8,11,14,17-pentaenoate

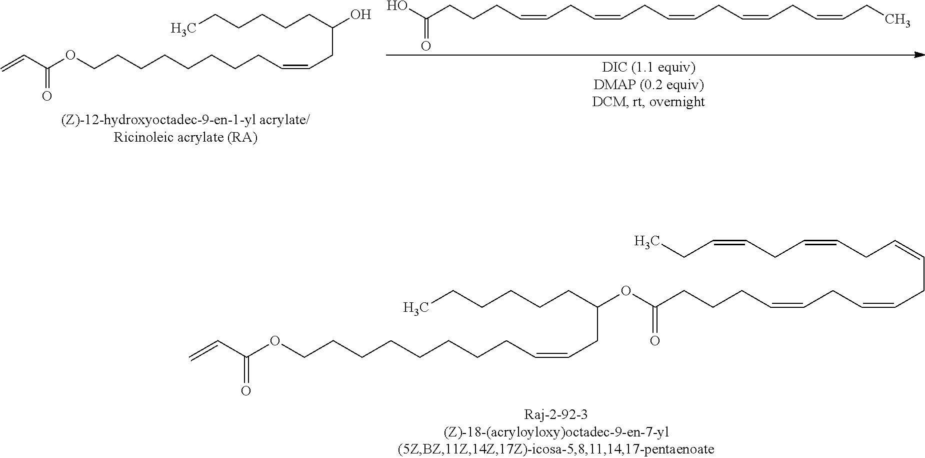 [SMILES: C=CC(=O)OCCCCCCCC/C=C\CC(O)CCCCCC ; CC/C=C\C/C=C\C/C=C\C/C=C\C/C=C\CCCC(=O)O ; C=CC(=O)OCCCCCCCC/C=C\CC(CCCCCC)OC(=O)CCC/C=C\C/C=C\C/C=C\C/C=C\C/C=C\CC]

To an oven dried two neck 50 mL round bottom flask under argon atmosphere were added cis-5,8,11,14,17-Eicosapentaenoic acid (1.4 g, 4.5 mmol, 1.5 equiv), N,N′-Diisopropylcarbodiimide (DIC) (0.7 mL, 4.5 mmol, 1.5 equiv) and DMAP (75 mg, 0.6 mmol, 0.2 equiv) followed by 10 mL anhydrous DCM. The reaction mixture was stirred at room temperature. After 30 min Ricinoleic acrylate (RA) (1.0 g, 3.0 mmol, 1.0 equiv, in 5 mL DCM) was added slowly to the above reaction mixture over 10 min and stirred at room temperature overnight. The crude reaction was quenched with sat. NaHCO₃ and extracted with DCM for 3 times (3×100 mL). The combined organic layers were washed with brine, dried over anhydrous MgSO₄, and concentrated in vacuo. Crude products were purified via SiO₂ gel flash column chromatography using hexanes and ethyl acetate as eluent at 30:1 to afford (Z)-18-(acryloyloxy)octadec-9-en-7-yl (5Z,8Z,11Z,14Z,17Z)-icosa-5,8,11,14,17-pentaenoate Yield: 1.5 g, 78%.

¹H-NMR (500 MHz, CDCl₃): δ 6.40 (dd J1=17.5 Hz, J2=1.5 Hz, 1H), 6.12 (dd, J1=17.5 Hz, J2=10.5 Hz, 1H), 5.82 (dd, J1=10.0 Hz, J2=1.5 Hz, 1H), 5.32-5.52 (m, 12H), 4.88 (m, 1H), 4.16 (t, J=7.0 Hz, 2H), 2.84-2.87 (m, 8H), 2.26-2.36 (m, 4H), 2.02-2.15 (m, 6H), 1.67-1.75 (m, 4H), 1.53-1.59 (m, 4H), 1.29-1.40 (m, 18H), 0.98 (t, J=7.5 Hz, 3H), 0.88 (t, J=7.0 Hz, 3H).

¹³C-NMR (125 MHz, CDCl₃): δ 173.3, 166.4, 132.6, 132.1, 130.4, 129.1, 128.74, 128.66, 128.6, 128.3, 128.14, 128.12, 127.9, 127.0, 124.3, 73.8, 64.7, 34.1, 33.6, 32.0, 31.8, 29.6, 29.4, 29.24, 29.17, 28.63, 27.4, 26.7, 25.59, 25.64, 25.55, 25.38, 25.0, 22.6, 20.6, 14.3, 14.1.

### Synthesis of (Z)-18(acryloyloxy)octadec-9-en-7-yl (9Z,12Z,15Z)-octadeca-9,12,15-trienoate

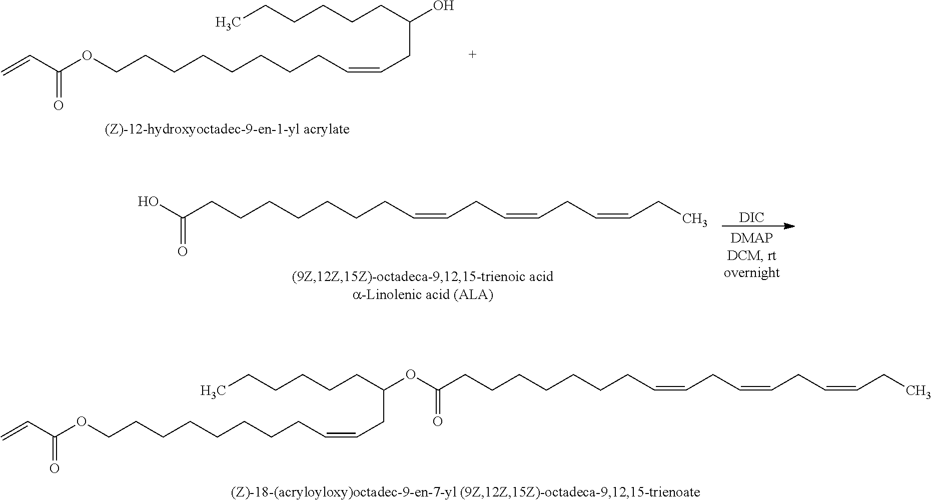

To an oven dried two neck 50 mL round bottom flask under argon atmosphere containing cis-(9Z,12Z,15Z)-octadeca-9,12,15-trienoic acid (2.47 g, 8.9 mmol, 1.5 equiv), N,N′-Diisopropylcarbodiimide (DIC) (1.4 mL, 8.9 mmol, 1.5 equiv) and DMAP (145 mg, 1.2 mmol, 0.2 equiv) was added 20 mL anhydrous DCM stirred the reaction mixture at room temperature. After 30 min Ricinoleic acrylate (RA) (2.0 g, 5.9 mmol, 1.0 equiv, in 10 mL DCM) was added slowly to the above reaction mixture over 10 min and stirred at room temperature for overnight. The crude reaction was quenched with sat. NaHCO₃ and extracted with DCM for 3 times (3×100 mL). The combined organic layers were washed with brine, dried over anhydrous MgSO₄, and concentrated in vacuo. Crude products were purified via SiO₂ gel flash column chromatography using hexanes and Ethyl acetate as eluent at 30:1 to afford (Z)-18(acryloyloxy)octadic-9-en-7-yl (9Z,12Z,15Z)-octadeca-9,12,15-trienoate Yield: 3.2 g, 89%.

¹H-NMR (500 MHz, CDCl₃): δ 6.42 (dd J1=17.4 Hz, J2=1.4 Hz, 1H), 6.14 (dd, J1=17.3 Hz, J2=10.4 Hz, 1H), 5.83 (dd, J1=10.4 Hz, J2=1.4 Hz, 1H), 5.54-5.32 (m, 7H), 4.90 (p, J=6.2 Hz, 1H), 4.17 (t, J=6.7 Hz, 2H), 2.89-2.77 (m, 4H), 2.30 (qd, J1=7.7 Hz, J2=2.7 Hz, 4H), 2.19-1.92 (m, 6H), 1.74-1.59 (m, 4H), 1.54 (t, J=6.7 Hz, 2H), 1.47-1.18 (m, 26H), 1.00 (t, J=7.5 Hz, 3H), 0.94 (t, J=7.0 Hz, 3H).

¹³C-NMR (125 MHz, CDCl₃): δ 173.6, 166.4, 132.6, 132.0, 130.4, 130.3, 128.7, 128.30, 128.26, 127.7, 127.1, 124.3, 73.7, 64.7, 34.7, 33.7, 32.0, 31.8, 29.61, 29.58, 29.4, 29.3, 29.23, 29.17, 28.6, 27.4, 27.2, 25.9, 25.6, 25.5, 25.4, 25.1, 22.6, 20.6, 14.3, 14.1.

### Synthesis of (Z)-18(acryloyloxy)octadic-9-en-7-yl dec-9-enoate

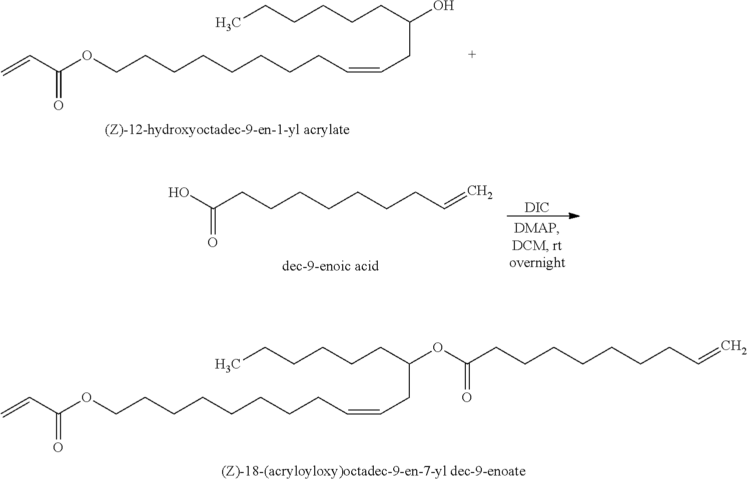

To an oven dried two neck 50 mL round bottom flask under argon atmosphere containing dec-9-enoic acid (1.13 g, 6.7 mmol, 1.5 equiv), N,N′-Diisopropylcarbodiimide (DIC) (1.1 mL, 6.7 mmol, 1.5 equiv) and DMAP (108 mg, 0.89 mmol, 0.2 equiv) was added 15 mL anhydrous DCM. The reaction mixture was stirred at room temperature. After 30 min Ricinoleic acrylate (RA) (1.5 g, 4.4 mmol, 1.0 equiv, in 10 mL DCM) was added slowly to the above reaction mixture over 10 min and stirred at room temperature for overnight. The crude reaction was quenched with sat. NaHCO₃ and extracted with DCM for 3 times (3×100 mL). The combined organic layers were washed with brine, dried over anhydrous MgSO₄, and concentrated in vacuo. Crude products were purified via SiO₂ gel flash column chromatography using hexanes and ethyl acetate as eluent at 30:1 to afford (Z)-18(acryloyloxy)octadic-9-en-7-yl dec-9-enoate Yield: 1.91 g, 87.8%.

¹H-NMR (500 MHz, CDCl₃): δ 6.42 (dd J1=17.4 Hz, J2=1.4 Hz, 1H), 6.14 (dd, J1=17.3 Hz, J2=10.4 Hz, 1H), 5.90-5.77 (m, 1H), 5.54-5.44 (m, 1H), 5.40-5.28 (m, 1H), 5.07-4.86 (m, 3H), 4.17 (t, J=6.7 Hz, 2H), 2.29 (t, J=7.5 Hz, 4H), 2.05 (p, J=7.0 Hz, 4H), 1.74-1.59 (m, 5H), 1.55 (q, J=7.1 Hz, 2H), 1.45-1.15 (m, 26H), 0.90 (t, J=7.0 Hz, 3H).

¹³C-NMR (125 MHz, CDCl₃): δ 173.6, 166.4, 139.1, 132.6, 130.4, 128.7, 124.3, 114.2, 73.7, 64.7, 34.7, 33.8, 33.7, 32.0, 31.8, 29.6, 29.44, 29.24, 29.17, 29.15, 29.13, 29.0, 28.9, 28.6, 27.4, 25.9, 25.4, 25.1, 22.6, 14.1.

### Synthesis of (Z)-18(acryloyloxy)octadec-9-en-7-yl (6Z,9Z,12Z)-octadeca-6,9,12-trienoate

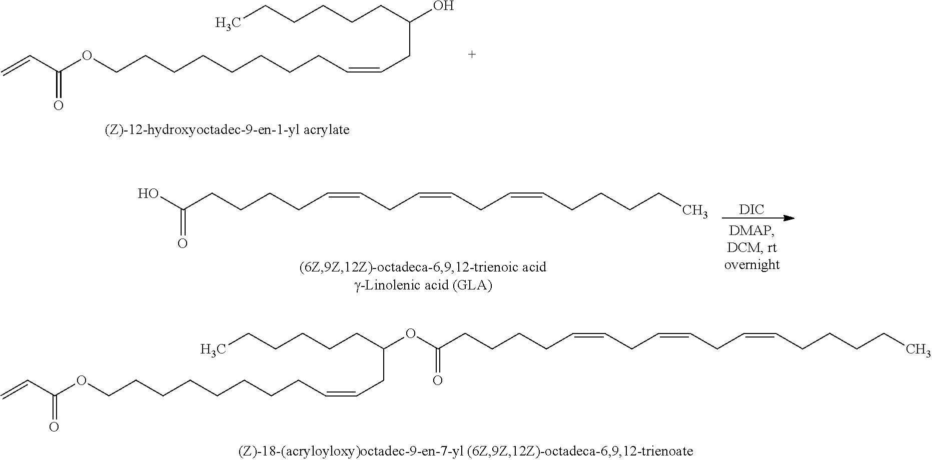

To an oven dried two neck 50 mL round bottom flask under argon atmosphere containing cis-(6Z,9Z,12Z)-octadeca-6,9,12-trienoic acid (3.4 g, 12.2 mmol, 1.5 equiv), N,N′-Diisopropylcarbodiimide (DIC) (1.9 mL, 12.2 mmol, 1.5 equiv) and DMAP (199 mg, 1.6 mmol, 0.2 equiv) was added 20 mL anhydrous DCM stirred the reaction mixture at room temperature. After 30 min, Ricinoleic acrylate (RA) (2.75 g, 8.1 mmol, 1.0 equiv, in 10 mL DCM) was added slowly to the above reaction mixture over 10 min and stirred at room temperature for overnight. The crude reaction was quenched with sat. NaHCO₃ and extracted with DCM for 3 times (3×100 mL). The combined organic layers were washed with brine, dried over anhydrous MgSO₄, and concentrated in vacuo. Crude products were purified via SiO₂ gel flash column chromatography using hexanes and ethyl acetate as eluent at 30:1 to afford (Z)-18(acryloyloxy)octadec-9-en-7-yl (6Z,9Z,12Z)-octadeca-6,9,12-trienoate Yield: 4.3 g, 87.3%.

¹H-NMR (500 MHz, CDCl₃): δ 6.42 (dd J1=17.3 Hz, J2=1.5 Hz, 1H), 6.14 (dd, J1=17.4 Hz, J2=10.4 Hz, 1H), 5.84 (dd, J1=10.4 Hz, J2=1.5 Hz, 1H), 5.55-5.28 (m, 8H), 4.91 (p, J=6.3 Hz, 1H), 4.17 (t, J=6.7 Hz, 2H), 2.83 (t, J=6.0 Hz, 4H), 2.30 (td, J1=7.3 Hz, J2=4.0 Hz, 4H), 2.16-1.95 (m, 6H), 1.76-1.62 (m, 4H), 1.62-1.50 (m, 3H), 1.49-1.14 (m, 26H), 0.91 (t, J=7.0 Hz, 6H).

¹³C-NMR (125 MHz, CDCl₃): δ 173.4, 166.4, 132.6, 130.5, 130.4, 129.7, 128.7, 128.4, 128.2, 128.1, 127.6, 124.3, 73.8, 64.7, 34.6, 33.7, 32.0, 31.8, 31.5, 29.6, 29.4, 29.34, 29.25, 29.17, 29.15, 28.6, 27.4, 27.2, 25.9, 25.6, 25.4, 24.8, 22.6, 14.1.

### General Procedure for Michael Addition Reaction with Ester Derived Ricinoleic Acrylate

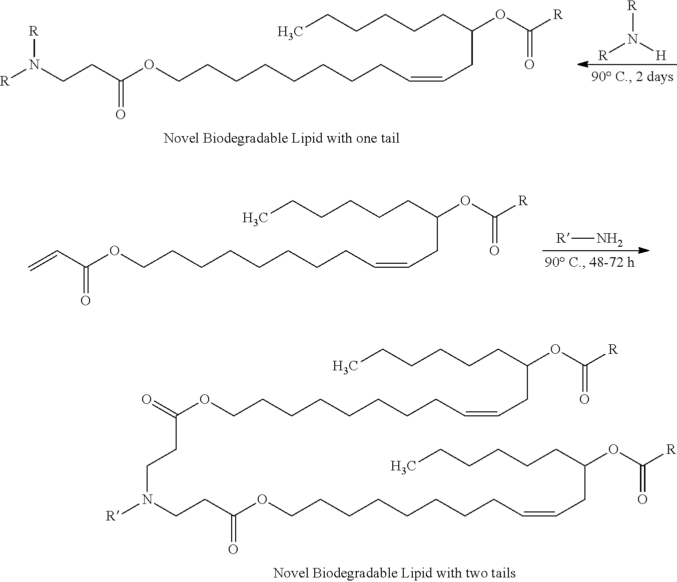

Into a 1-dram scintillation vial equipped with magnetic stir bar was placed ester derived ricinoleic acrylate ester (0.5 mmol, 2.5 equiv) and aliphatic amine (0.2 mmol, 1.0 equiv). Generally, the lipids were synthesized with a molar ratio of amine:ricinoleic acrylate ester in 1:2.5 and 1:1.25 equiv for primary and secondary amines respectively. The vial was sealed with a silicone-lined screwcap and stirred at 90° C. for 2-3 days or until complete conversion of the starting material amine. Further purification was achieved by flash column chromatography on an Isco Combi flash system eluting with a gradient (0-100%) Ultra (3% concentrated ammonium hydroxide, 22% methanol, 75% dichloromethane): dichloromethane to afford ionizable lipids (MR).

### Synthesis of (9Z,9′Z)-((3,3′-((3-(diethylamino)propyl)azanediyl)bis(propanoyl))bis(oxy))bis(202ctadic-9-ene-18,7-diyl) (5Z,5′Z,8Z,8′Z,11Z,11′Z,14Z,14′Z,17Z,17′Z)-bis(icosa-5,8,11,14,17-pentaenoate)

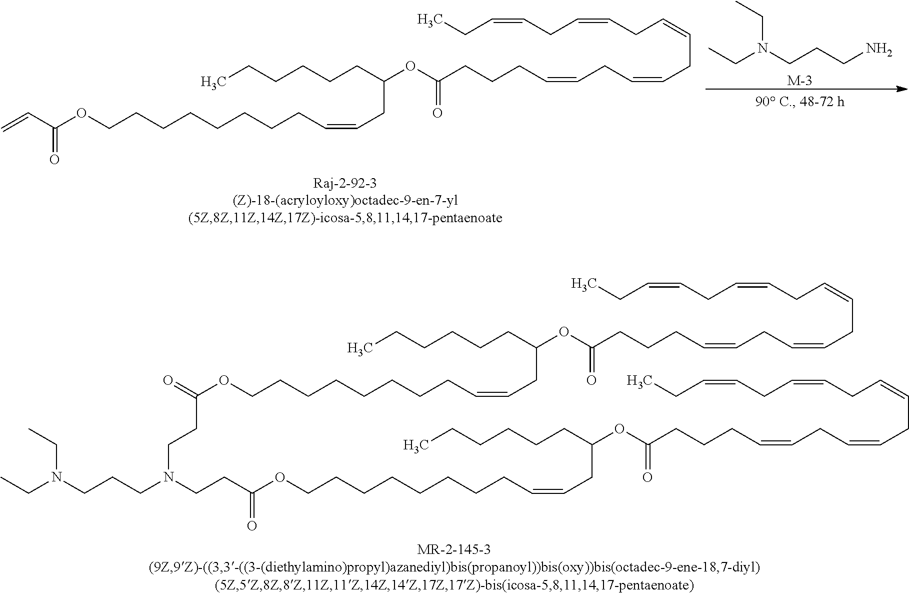

Into a 1-dram scintillation vial equipped with magnetic stir bar was placed (Z)-18-(acryloyloxy)octadic-9-en-7-yl (5Z,8Z,11Z,14Z,17Z)-icosa-5,8,11,14,17-pentaenoate (310 mg, 0.5 mmol, 2.5 equiv) and N,N-diethylpropane-1,3-diamine (26 mg, 0.2 mmol, 1.0 equiv). The vial was sealed with a silicone-lined screwcap and stirred at 90° C. for 2-3 days or until complete conversion of the starting material amine. Further purification was achieved by flash column chromatography on an Isco Combi flash system eluting with a gradient (0-100%) Ultra (3% concentrated ammonium hydroxide, 22% methanol, 75% dichloromethane): dichloromethane to afford (9Z,9′Z)-((3,3′-((3-(diethylamino)propyl)azanediyl)bis(propanoyl))bis(oxy))bis(octadic-9-ene-18,7-diyl) (5Z,5′Z,8Z,8′Z,11Z,11′Z,14Z,14′Z,17Z,17′Z)-bis(icosa-5,8,11,14,17-pentaenoate) as a colorless liquid (yield 222 mg, 81%)

¹H-NMR (500 MHz, CDCl₃): δ 5.31-5.51 (m, 24H), 4.88-4.93 (m, 2H), 4.06 (t, J=6.5 Hz, 4H), 2.78-2.87 (m, 18H), 2.40-2.55 (m, 12H), 2.29-2.33 (m, 8H), 2.02-2.15 (m, 12H), 1.69-1.75 (m, 4H), 1.55-1.65 (m, 16H), 1.29-1.39 (m, 36H), 0.98-1.05 (m, 12H), 0.88 (t, J=7.0 Hz, 6H)

¹³C-NMR (125 MHz, CDCl₃): δ 173.3, 172.8, 132.6, 132.1, 129.1, 128.7, 128.6, 128.3, 128.14, 128.12, 127.9, 127.0, 124.3, 73.8, 64.6, 51.9, 50.9, 49.2, 46.9, 34.1, 33.6, 32.6, 31.9, 31.8, 29.6, 29.5, 29.33, 29.29, 29.2, 28.7, 27.4, 26.7, 25.9, 25.64, 25.55, 25.4, 25.0, 22.6, 20.6, 14.3, 14.1, 11.7

### Synthesis of RML-66

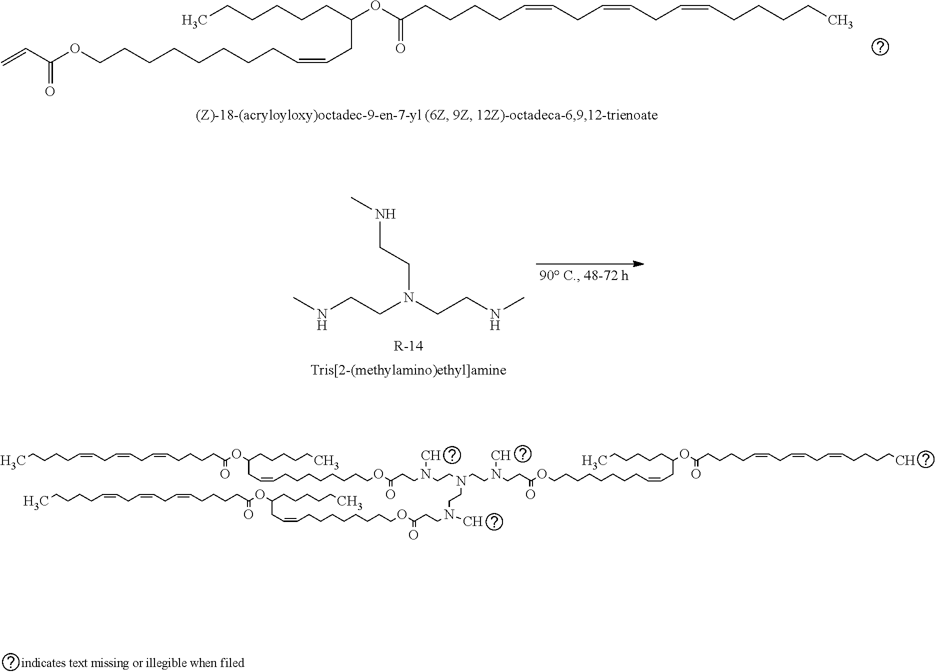

Into a 1-dram scintillation vial equipped with magnetic stir bar was placed (Z)-18(acryloyloxy)Octadic-9-en-7-yl (6Z,9Z,12Z)-octadeca-6,9,12-trienoate (255 mg, 0.42 mmol, 4.0 equiv) and tris [2-(methylamino)ethyl]amine (20 mg, 0.11 mmol, 1.0 equiv). The vial was sealed with a silicone-lined screwcap and stirred at 90° C. for 2-3 days or until complete conversion of the starting material amine. Further purification was achieved by flash column chromatography on an Isco Combi flash system eluting with a gradient (0-100%) Ultra (3% concentrated ammonium hydroxide, 22% methanol, 75% dichloromethane): dichloromethane to afford RML-66 as a colorless liquid (yield 156 mg, 74%).

¹H-NMR (500 MHz, CDCl₃): δ 5.50-5.31 (m, 24H), 4.93-4.88 (m, 3H), 4.09 (t, J=6.5 Hz, 6H), 2.84-2.74 (m, 20H), 2.64-2.59 (m, 6H), 2.54-2.43 (m, 11H), 2.35-2.22 (m, 20H), 2.12-2.01 (m, 18H), 1.69-1.60 (m, 12H), 1.56-1.54. (m, 6H), 1.45-1.23 (m, 78H), 0.92-0.88 (m, 18H).

¹³C NMR (126 MHz, CDCl₃) δ 173.4, 172.6, 132.6, 130.5, 129.7, 128.4, 128.2, 128.1, 127.6, 124.3, 73.7, 64.6, 55.6, 53.3, 53.0, 42.4, 34.6, 33.6, 32.5, 32.0, 31.8, 31.5, 29.6, 29.5, 29.34, 29.31, 29.28, 29.2, 29.1, 28.7, 27.4, 27.2, 26.9, 25.9, 25.6, 25.4, 24.8, 22.6, 14.1.

### Synthesis of (Z)-12-(((4-nitrophenoxy)carbonyl)oxy)octadic-9-en-1-yl acrylate

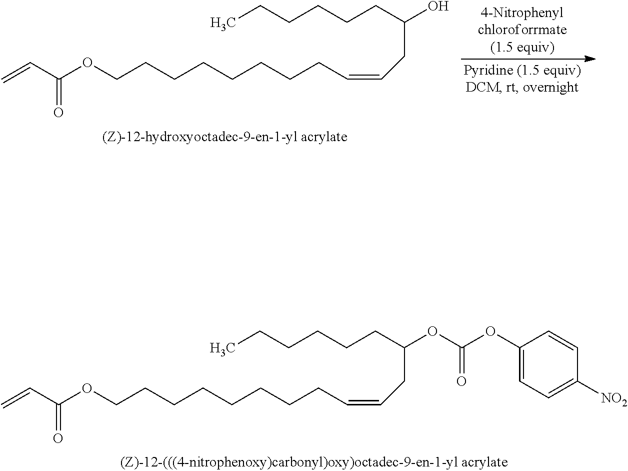 [SMILES: C=CC(=O)OCCCCCCCC/C=C\CC(O)CCCCCC ; C=CC(=O)OCCCCCCCC/C=C\CC(CCCCCC)OC(=O)Oc1ccc([N+](=O)[O-])cc1]

To a 100 mL round bottom flask taken 4-Nitrophenyl chloroformate (1.8 g, 9.0 mmol), and (Z)-12-hydroxyoctadec-9-en-1-yl acrylate (2.0 g, 6.0 mmol) and DCM (30 mL) was added under argon atmosphere. After dissolving all the starting materials, pyridine (0.72 mL, 9.0 mmol) was slowly added drop wise and the reaction mixture stirred at room temperature for overnight or until completion of the starting material. Reaction mass was diluted with brine (20 mL) and extracted with dichloromethane (3×100 mL) dried over anhydrous. Na₂SO₄ and concentrated under reduced pressure. Crude products were purified via SiO2 gel flash column chromatography using hexanes and Ethyl acetate as eluent at 25:1 to afford (Z)-12-(((4-nitrophenoxy)carbonyl)oxy)octadic-9-en-1-yl acrylate as a light yellow in color. Yield: 2.7 g, 88%.

¹H-NMR (500 MHz, CDCl₃): δ 8.29 (d, J=9.0 Hz, 2H), 7.38 (d, J=9.0 Hz, 2H), 6.40 (dd, J1=17.0 Hz, J2=1.5 Hz, 1H), 6.11 (dd, J1=17.5 Hz, J2=10.5 Hz, 1H), 5.82 (dd, J1=10.5 Hz, J2=1.5 Hz, 1H), 5.55-5.61 (m, 1H), 5.39-5.43 (m, 1H), 4.83-4.88 (m, 1H), 4.15 (t, J=7.0 Hz, 2H), 2.39-2.53 (m, 2H), 2.06 (q, J=7.0 Hz, 2H), 1.65-1.76 (m, 4H), 1.28-1.44 (m, 18H), 0.90 (t, J=7.0 Hz, 3H).

¹³C-NMR (125 MHz, CDCl₃): δ 166.4, 155.7, 152.3, 145.3, 133.7, 130.7, 130.5, 128.6, 125.3, 123.2, 121.8, 80.6, 64.7, 35.5, 31.9, 31.7, 29.5, 29.4, 29.23, 29.20, 29.1, 28.6, 27.4, 25.9, 25.2, 22.6, 14.1.

### General Procedure for Synthesis of Carbonates of Ricinoleic Acrylates

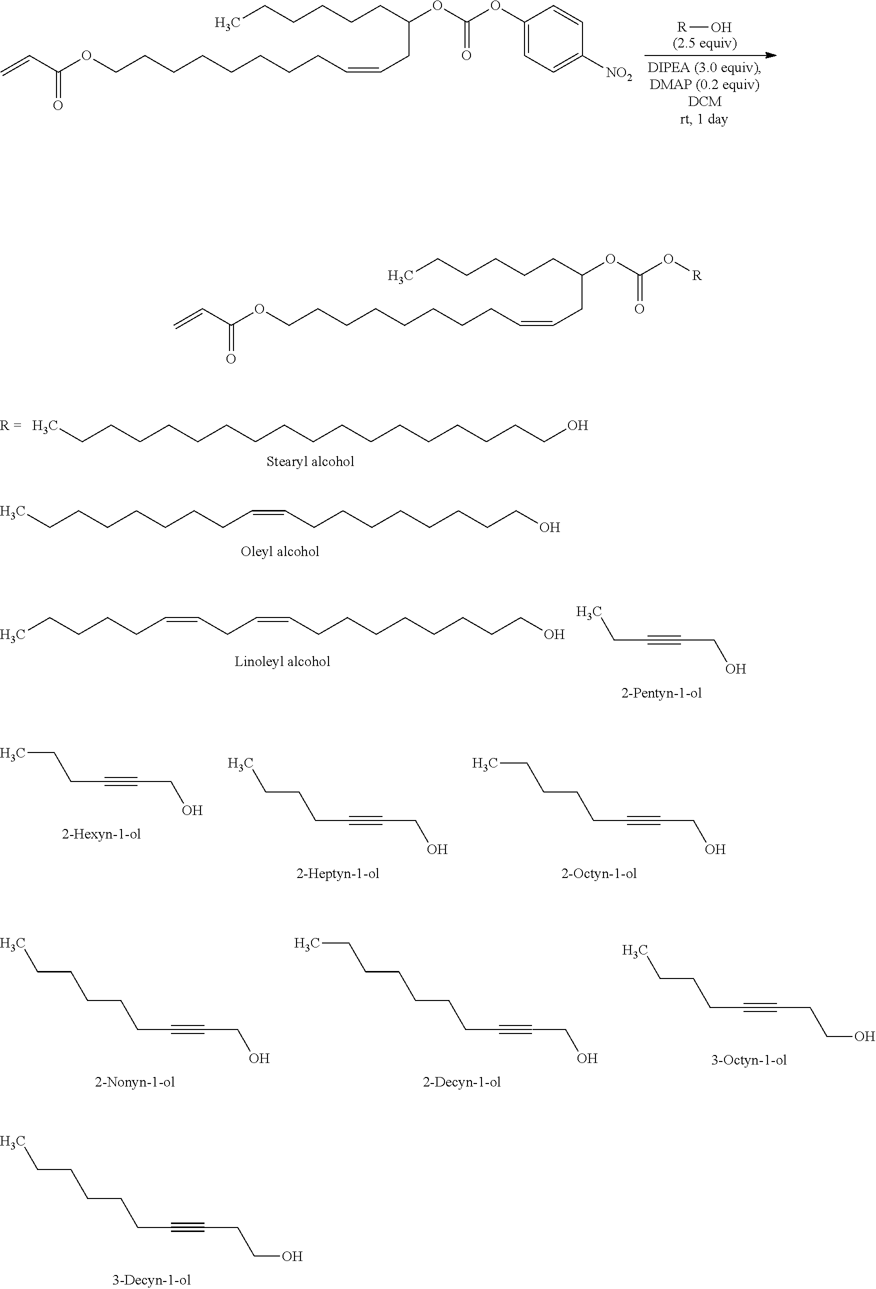

To a round bottom flask containing 4-Nitrophenyl chloroformate (1.0 mmol, 1.0 equiv), DMAP (0.2 mmol, 0.2 equiv) and aliphatic alcohol (2.5 mmol, 2.5 equiv) in DCM (20 mL) were added. After dissolving all the starting materials DIPEA (3.0 mmol, 3.0 equiv) was slowly added drop wise and the reaction mixture stirred at room temperature overnight or until conversion of the starting material. Reaction mass was diluted with brine (20 mL) and extracted with dichloromethane (3×100 mL) dried over anhydrous Na₂SO₄ and concentrated under reduced pressure. The organic layer was dried over anhydrous MgSO₄ and concentrated in vacuo. Crude products were purified via SiO₂ gel flash column chromatography using hexanes and ethyl acetate as eluent at 30:1 to afford ricinoleic carbonates as a colorless liquid.

### Synthesis of (Z)-12-(((non-2-yn-1-yloxy)carbonyl)oxy)octadec-9-en-1-yl acrylate

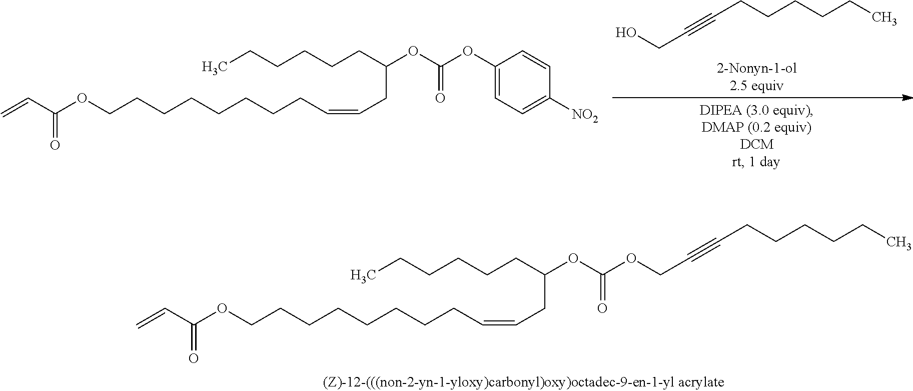

To a round bottom flask containing 4-Nitrophenyl chloroformate (500 mg, 1.0 mmol, 1.0 equiv), DMAP (24 mg, 0.2 mmol) and 2-Nonyn-1-ol (350 mg, 2.5 mmol, 2.5 equiv) in DCM (20 mL) were added. After dissolving all the starting materials DIPEA (0.52 mL, 3.0 mmol) was slowly added drop wise and the reaction mixture stirred at room temperature overnight or until conversion of the starting material. Reaction mass was diluted with brine (20 mL) and extracted with dichloromethane (3×100 mL). Crude organic layer was dried over anhydrous MgSO₄ and concentrated in vacuo. Crude products were purified via SiO₂ gel flash column chromatography using hexanes and ethyl acetate as eluent at 30:1 to afford (Z)-12-(((non-2-yn-1-yloxy)carbonyl)oxy)octadec-9-en-1-yl acrylate as a colorless liquid; Yield: 522 mg, 83%.

¹H-NMR (500 MHz, CDCl₃): δ 6.44 (dd, J1=17.5 Hz, J2=1.5 Hz, 1H), 6.11 (dd, J1=17.5 Hz, J2=10.5 Hz, 1H), 5.82 (dd, J1=10.5 Hz, J2=1.5 Hz, 1H), 5.49-5.54 (m, 1H), 5.35-5.39 (m, 1H), 4.68-4.74 (m, 3H), 4.16 (t, J=6.5 Hz, 2H), 2.31-2.43 (m, 2H), 1.67-1.71 (m, 2H), 1.58-1.63 (m, 2H), 1.49-1.56 (m, 2H), 1.24-1.42 (m, 23H), 0.88 (m, 6H).

¹³C-NMR (125 MHz, CDCl₃): δ 166.4, 154.6, 133.1, 130.4, 128.7, 123.7, 88.4, 79.0, 73.5, 64.7, 56.0, 33.5, 31.9, 31.7, 31.3, 29.5, 29.4, 29.2, 29.1, 28.6, 28.5, 28.3, 27.4, 25.9, 25.2, 22.6, 22.5, 18.8, 14.07, 14.05

### General Procedure for Michael Addition Reaction with Carbonate Derived Ricinoleic Acrylate

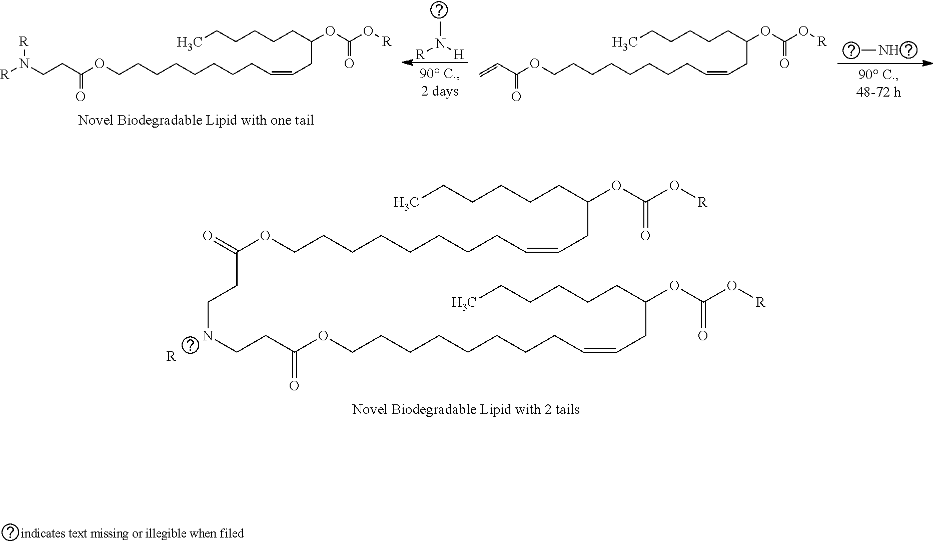

Into a 1-dram scintillation vial equipped with magnetic stir bar was placed carbonate derived Ricinoleic acrylate (0.5 mmol, 2.5 equiv) and Aliphatic amine (0.2 mmol, 1.0 equiv). Generally, the lipids were synthesized with a molar ratio of amine/Ricinoleic acrylate carbonates in 1:2.5 and 1:1.25 equiv for primary and secondary amines respectively. The vial was sealed with a silicone-lined screwcap and stirred at 90° C. for 2-3 days or until complete conversion of the starting material amine. Further purification was achieved by flash column chromatography on an Isco Combiflash system eluting with a gradient (0-100%) Ultra (3% concentrated ammonium hydroxide, 22% methanol, 75% dichloromethane): dichloromethane to afford carbonate derived Ricinoleic acrylate based ionizable lipids (RCB).

### Synthesis of bis((Z)-12-(((non-2-yn-1-yloxy)carbonyl)oxy)octadec-9-en-¹-yl) 3,3′-((2-(diethylamino)ethyl)azanediyl)dipropionate

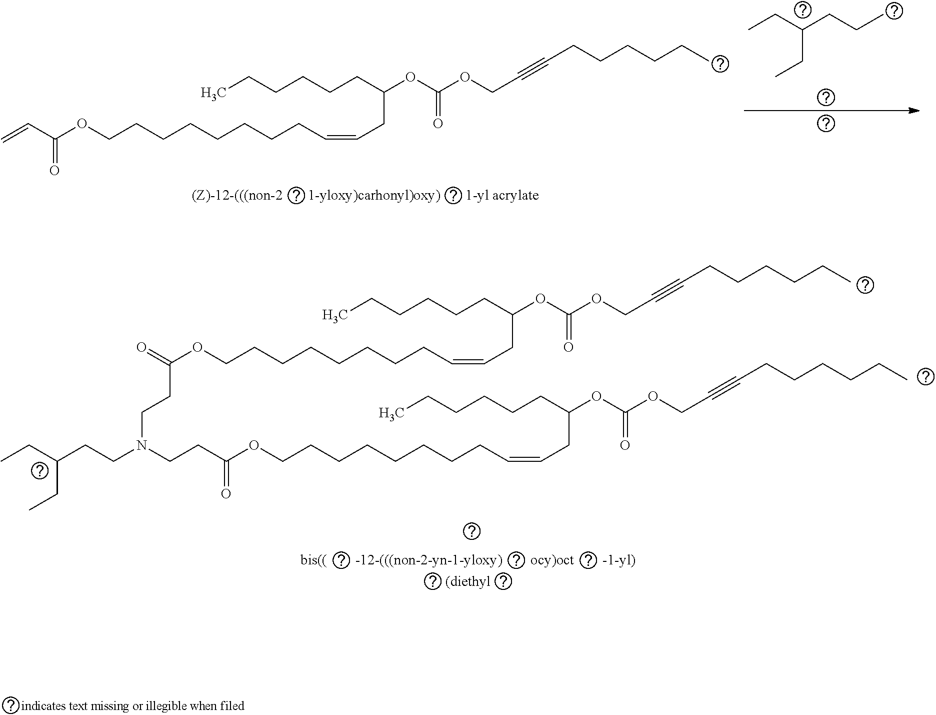

Into a 1-dram scintillation vial equipped with magnetic stir bar was placed (Z)-12-(((non-2-yn-1-yloxy)carbonyl)oxy)octadec-9-en-1-yl acrylate (250 mg, 0.5 mmol, 2.5 equiv) and N,N-diethylethane-1,2-diamine (23 mg, 0.2 mmol, 1.0 equiv). The vial was sealed with a silicone-lined screwcap and stirred at 90° C. for 2-3 days or until complete conversion of the starting material amine. Further purification was achieved by flash column chromatography on an Isco Combiflash system eluting with a gradient (0-100%) Ultra (3% concentrated ammonium hydroxide, 22% methanol, 75% dichloromethane): dichloromethane to afford bis((Z)-12-(((non-2-yn-1-yloxy)carbonyl)oxy)octadec-9-en-1-yl) 3,3′-((2-(diethylamino)ethyl)azanediyl)dipropionate as a colorless oil (Yield: 176 mg, 79%).

¹H-NMR (500 MHz, CDCl₃): δ 5.48-5.54 (m, 2H), 5.34-5.39 (m, 2H), 4.69-4.76 (m, 6H), 4.06 (t, J=7.0 Hz, 4H), 2.80 (t, J=7.0 Hz, 4H), 2.52-2.57 (m, 8H), 2.45-2.48 (m, 4H), 2.31-2.43 (m, 4H), 2.21-2.25 (m, 4H), 2.02-2.07 (m, 4H), 1.61-1.67 (m, 1H), 1.51-1.59 (m, 4H), 1.29-1.42 (m, 46H), 1.03 (t, J=7.0 Hz, 6H), 0.88 (m, 12H).

¹³C-NMR (125 MHz, CDCl₃): δ 172.7, 154.6, 133.1, 123.6, 88.4, 79.0, 73.5, 64.6, 56.0, 52.2, 51.3, 49.8, 47.5, 33.5, 32.7, 31.9, 31.7, 31.3, 29.6, 29.5, 29.5, 29.3, 29.1, 28.6, 28.5, 28.3, 27.4, 25.9, 25.2, 22.6, 22.5, 18.8, 14.07, 14.05, 11.8.

### Synthesis of RCB-01-223-3

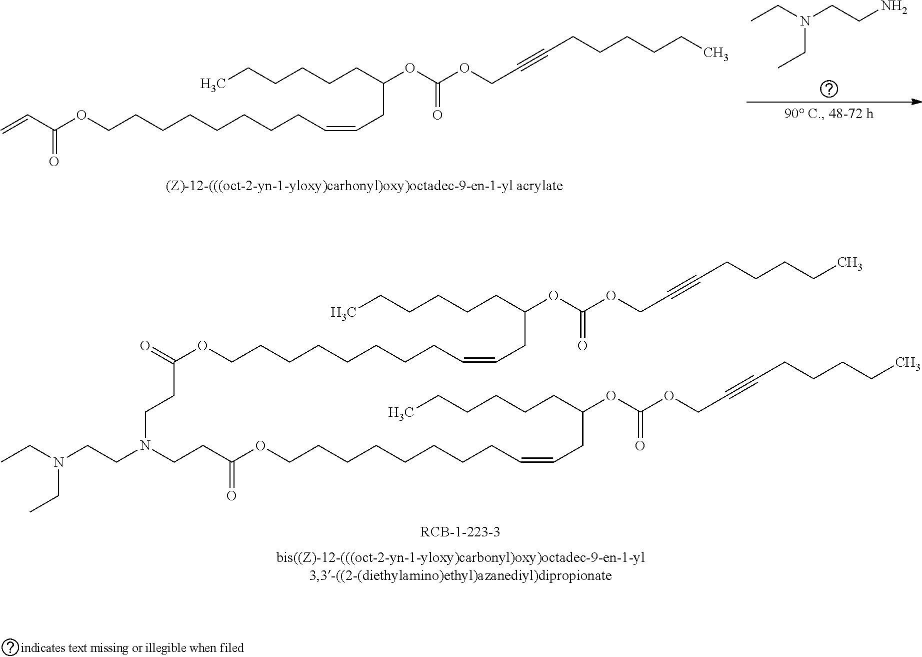

Into a 1-dram scintillation vial equipped with magnetic stir bar was placed (Z)-12-(((oct-2-yn-1-yloxy)octadiccarbonyl)oxy)206ctadic-9-en-1-yl acrylate (243 mg, 0.5 mmol, 2.5 equiv) and N,N-diethylethane-1,2-diamine (23 mg, 0.2 mmol, 1.0 equiv). The vial was sealed with a silicone-lined screwcap and stirred at 90° C. for 2-3 days or until complete conversion of the starting material amine. Further purification is achieved by flash column chromatography on an Isco Combiflash system eluting with a gradient (0-100%) Ultra (3% concentrated ammonium hydroxide, 22% methanol, 75% dichloromethane): dichloromethane to afford bis((Z)-12-(((oct-2-yn-1-yloxy)octadiccarbonyl)oxy)207ctadic-9-en-1-yl) 3,3′-((2-(diethylamino)ethyl)azanediyl)dipropionate as a colorless oil (Yield: 166 mg, 76%).

¹H NMR (500 MHz, CDCl₃) δ 5.56-5.45 (m, 2H), 5.41-5.32 (m, 2H), 4.72 (s, 6H), 4.07 (t, J=6.8 Hz, 4H), 2.82 (t, J=7.3 Hz, 4H), 2.55 (d, J=7.0 Hz, 8H), 2.46 (t, J=7.3 Hz, 4H), 2.44-2.29 (m, 5H), 2.27-2.18 (m, 4H), 2.09-1.98 (m, 4H), 1.63 (td, J1=14.5, J2=8.2 Hz, 10H), 1.53 (p, J=7.2 Hz, 5H), 1.46-1.18 (m, 45H), 1.04 (t, J=7.1 Hz, 6H), 0.96-0.83 (m, 12H).

¹³C NMR (126 MHz, CDCl₃) δ 172.7, 154.6, 133.1, 123.7, 88.4, 79.0, 73.5, 64.6, 56.0, 52.2, 51.3, 49.8, 47.5, 33.5, 32.7, 31.9, 31.7, 31.0, 31.0, 29.5, 29.5, 29.3, 29.1, 28.7, 28.1, 27.4, 26.0, 25.2, 22.6, 22.2, 18.7, 14.1, 14.0, 11.8.

### Synthesis of RCB-02-102-5

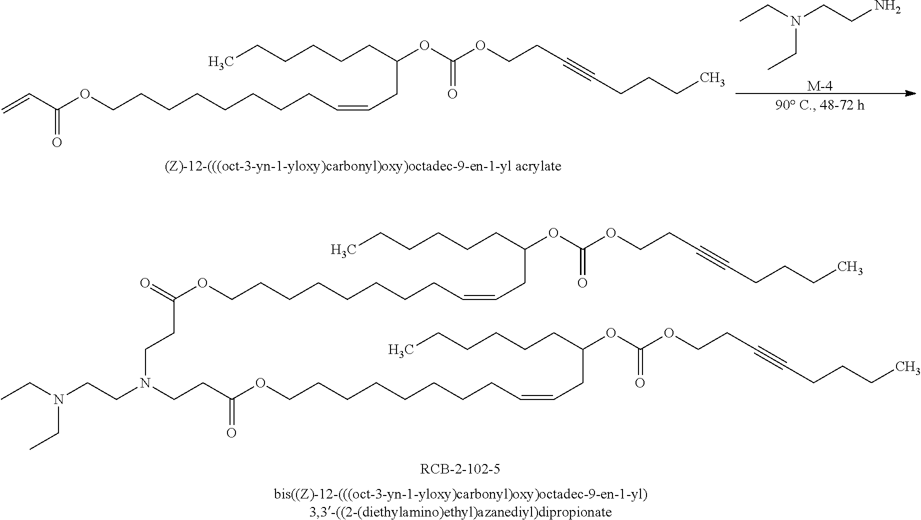

Into a 1-dram scintillation vial equipped with magnetic stir bar was placed (Z)-12-(((oct-3-yn-1-yloxy)octadiccarbonyl)oxy)octadec-9-en-1-yl acrylate (243 mg, 0.5 mmol, 2.5 equiv) and N,N-diethylethane-1,2-diamine (23.2 mg, 0.2 mmol, 1.0 equiv). The vial was sealed with a silicone-lined screwcap and stirred at 90° C. for 2-3 days or until complete conversion of the starting material amine. Further purification is achieved by flash column chromatography on an Isco Combiflash system eluting with a gradient (0-100%) Ultra (3% concentrated ammonium hydroxide, 22% methanol, 75% dichloromethane): dichloromethane to afford bis((Z)-12-(((oct-3-yn-1-yloxy)octadiccarbonyl)oxy)207ctadic-9-en-1-yl) 3,3′-((2-(diethylamino)ethyl)azanediyl)dipropionate as a colorless oil (Yield: 156 mg, 71%).

¹H NMR (500 MHz, CDCl₃) δ 5.59-5.46 (m, 2H), 5.46-5.33 (m, 2H), 4.79-4.64 (m, 2H), 4.19 (t, J=7.2 Hz, 4H), 4.07 (t, J=6.8 Hz, 4H), 2.82 (t, J=7.3 Hz, 4H), 2.55 (tt, J1=7.3, J2=2.6 Hz, 11H), 2.47 (t, J=7.3 Hz, 4H), 2.45-2.28 (m, 4H), 2.16 (tt, J1=7.0, J2=2.4 Hz, 4H), 2.11-1.99 (m, 4H), 1.61 (qd, J1=14.0, J2=7.8 Hz, 13H), 1.53-1.45 (m, 4H), 1.45-1.21 (m, 38H), 1.04 (t, J=7.1 Hz, 6H), 0.91 (dt, J1=11.5, J2=7.1 Hz, 12H).

¹³C NMR (126 MHz, CDCl₃) δ 172.7, 154.8, 133.1, 123.7, 82.3, 78.6, 74.9, 65.9, 64.6, 51.3, 49.8, 47.5, 33.6, 32.7, 31.9, 31.7, 31.0, 29.6, 29.5, 29.3, 29.1, 28.7, 27.4, 26.0, 25.2, 22.6, 21.9, 19.4, 18.4, 14.1, 13.6.

### Synthesis of RCB-02-76-3

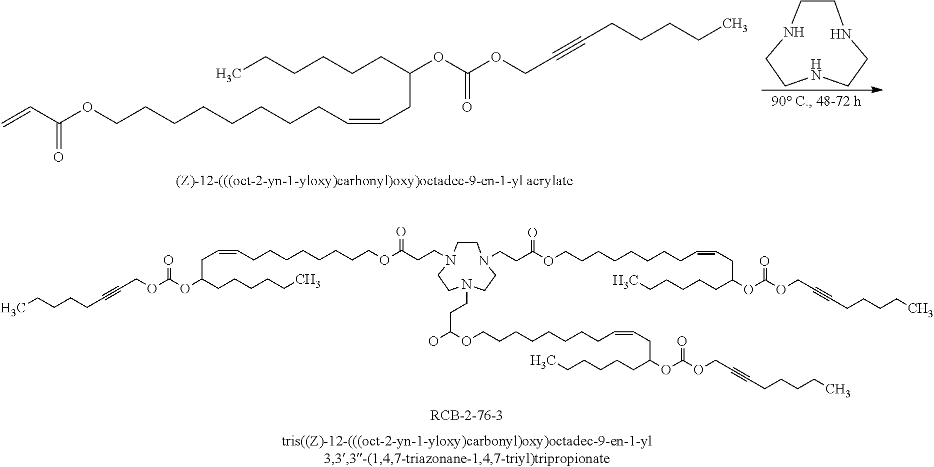

Into a 1-dram scintillation vial equipped with magnetic stir bar was placed (Z)-12-(((oct-2-yn-1-yloxy)octadiccarbonyl)oxy)octadec-9-en-1-yl acrylate (482 mg, 0.8 mmol, 4.0 equiv) and 1,4,7-Triazacyclononane (26 mg, 0.2 mmol, 1.0 equiv). The vial was sealed with a silicone-lined screwcap and stirred at 90° C. for 2-3 days or until complete conversion of the starting material amine. Further purification is achieved by flash column chromatography on an Isco Combiflash system eluting with a gradient (0-100%) Ultra (3% concentrated ammonium hydroxide, 22% methanol, 75% dichloromethane): dichloromethane to afford tris((Z)-12-(((oct-2-yn-1-yloxy)octadiccarbonyl)oxy)208ctadic-9-en-1-yl) 3,3′,3-(1,4,7-triazonane-1,4,7-triyl)tripropionate as a colorless oil (Yield: 256 mg, 66%).

¹H NMR (500 MHz, CDCl₃) δ 5.55-5.47 (m, 3H), 5.41-5.33 (m, 3H), 4.77-4.70 (m, 8H), 4.07 (t, J=6.8 Hz, 6H), 2.85 (t, J=7.3 Hz, 5H), 2.72 (s, 10H), 2.45 (t, J=7.3 Hz, 5H), 2.46-2.28 (m, 10H), 2.23 (tt, J1=7.2, J2=2.2 Hz, 6H), 2.09-2.01 (m, 6H), 1.74-1.58 (m, 12H), 1.54 (dq, J1=14.6, J2=6.7 Hz, 7H), 1.48-1.20 (m, 65H), 0.91 (dt, J1=10.0, J2=7.1 Hz, 18H).

¹³C NMR (126 MHz, CDCl₃) δ 172.9, 154.6, 133.1, 123.7, 88.4, 79.0, 73.5, 64.6, 56.0, 55.6, 54.2, 33.5, 31.9, 31.7, 31.0, 29.5, 29.5, 29.3, 29.1, 28.7, 28.1, 27.4, 26.0, 25.2, 22.6, 22.2, 18.7, 14.1, 14.0.

### Synthesis of RCB-02-104-1

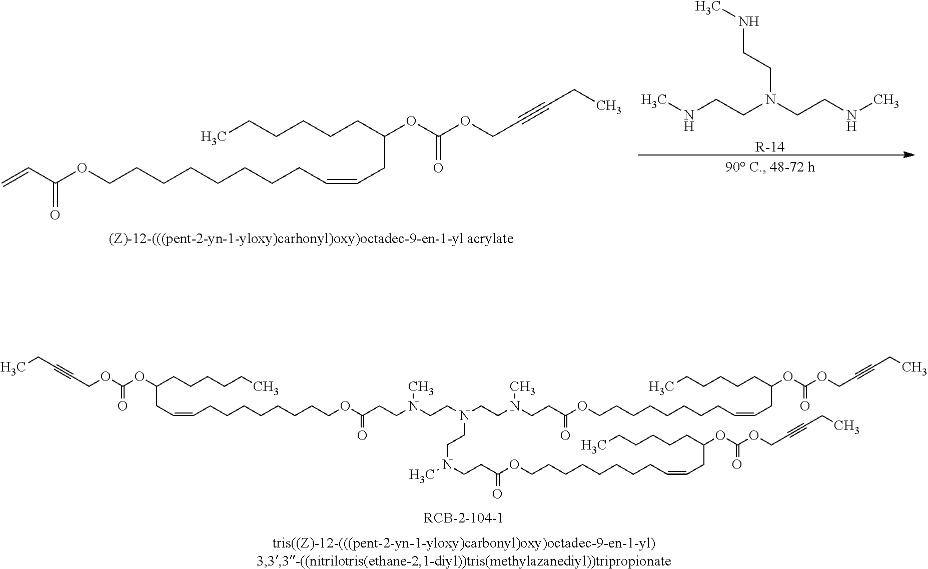

Into a 1-dram scintillation vial equipped with magnetic stir bar was placed (Z)-12-(((pent-2-yn-1-yloxy)octadiccarbonyl)oxy)octadec-9-en-1-yl acrylate (357 mg, 0.8 mmol, 4.0 equiv) and Tris[2-(methylamino)ethyl]amine (37.5 mg, 0.2 mmol, 1.0 equiv). The vial was sealed with a silicone-lined screwcap and stirred at 90° C. for 2-3 days or until complete conversion of the starting material amine. Further purification is achieved by flash column chromatography on an Isco Combiflash system eluting with a gradient (0-100%) Ultra (3% concentrated ammonium hydroxide, 22% methanol, 75% dichloromethane): dichloromethane to afford tris((Z)-12-(((pent-2-yn-1-yloxy)octadiccarbonyl)oxy)209ctadic-9-en-1-yl) 3,3′,3-((nitrilotris(ethane-2,1-diyl))tris(methylazanediyl))tripropionate as a colorless oil (Yield: 222 mg, 73%).

¹H NMR (500 MHz, CDCl₃) δ 5.58-5.44 (m, 3H), 5.44-5.33 (m, 3H), 4.79-4.69 (m, 8H), 4.08 (t, J=6.8 Hz, 6H), 2.73 (t, J=7.4 Hz, 6H), 2.62 (dd, J1=9.0, J2=5.7 Hz, 6H), 2.48 (t, J=7.3 Hz, 12H), 2.45-2.30 (m, 8H), 2.30-2.18 (m, 14H), 2.11-1.98 (m, 6H), 1.73-1.53 (m, 19H), 1.48-1.22 (m, 48H), 1.16 (t, J=7.5 Hz, 8H), 0.90 (t, J=6.8 Hz, 9H).

¹³C NMR (126 MHz, CDCl₃) δ 172.7, 154.6, 133.1, 123.7, 89.6, 79.0, 76.8, 72.9, 64.6, 56.0, 55.6, 53.3, 53.0, 42.4, 33.5, 32.5, 31.9, 31.7, 29.5, 29.5, 29.3, 29.1, 28.7, 27.4, 25.9, 25.2, 22.6, 14.1, 13.5, 12.5.

### Synthesis of RCB-02-113-2

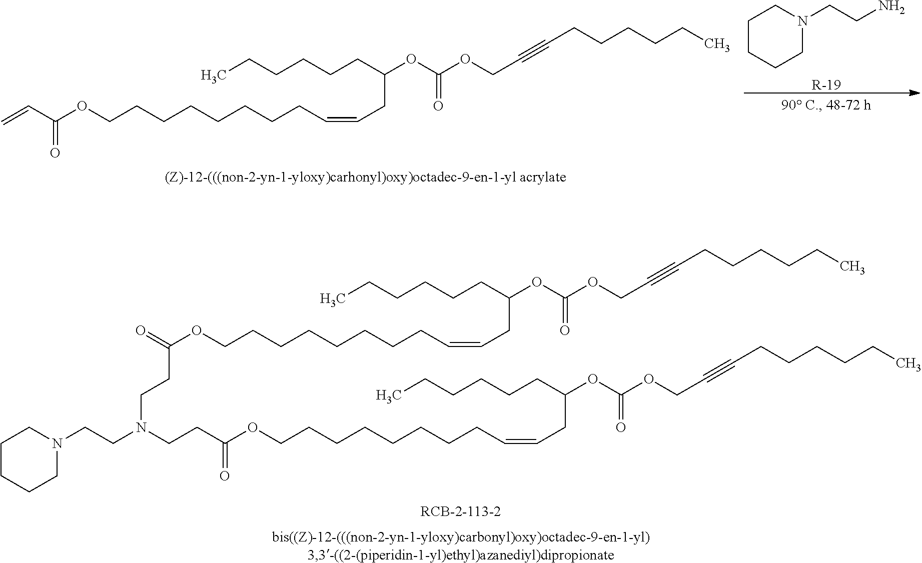

Into a 1-dram scintillation vial equipped with magnetic stir bar was placed (Z)-12-(((non-2-yn-1-yloxy)octadiccarbonyl)oxy)octadec-9-en-1-yl acrylate (254 mg, 0.5 mmol, 2.5 equiv) and 1-(2-Aminoethyl)piperidine (25.8 mg, 0.2 mmol, 1.0 equiv). The vial was sealed with a silicone-lined screwcap and stirred at 90° C. for 2-3 days or until complete conversion of the starting material amine. Further purification is achieved by flash column chromatography on an Isco Combiflash system eluting with a gradient (0-100%) Ultra (3% concentrated ammonium hydroxide, 22% methanol, 75% dichloromethane): dichloromethane to afford tris((Z)-12-(((pent-2-yn-1-yloxy)octadiccarbonyl)oxy)octadec-9-en-1-yl) 3,3′,3-((nitrilotris(ethane-2,1-diyl))tris(methylazanediyl))tripropionate as a colorless oil (Yield: 178 mg, 78%).

¹H NMR (500 MHz, CDCl₃) δ 5.57-5.47 (m, 2H), 5.43-5.34 (m, 2H), 4.79-4.70 (m, 6H), 4.07 (t, J=6.8 Hz, 4H), 2.82 (t, J=7.3 Hz, 4H), 2.62 (t, J=7.3 Hz, 2H), 2.46 (t, J=7.3 Hz, 4H), 2.44-2.30 (m, 8H), 2.23 (tt, J1=7.1, J2=2.2 Hz, 4H), 2.12-2.00 (m, 4H), 1.74-1.56 (m, 13H), 1.52 (p, J=7.2 Hz, 5H), 1.48-1.20 (m, 50H), 0.99-0.81 (m, 12H).

¹³C NMR (126 MHz, CDCl₃) δ 172.7, 154.6, 133.1, 123.7, 88.4, 79.0, 76.8, 73.5, 64.6, 57.4, 55.99, 55.1, 51.4, 49.8, 33.5, 32.7, 31.9, 31.7, 31.7, 31.3, 31.3, 29.5, 29.5, 29.3, 29.1, 28.7, 28.5, 28.35, 27.38, 26.02, 25.96, 25.2, 24.4, 22.6, 22.5, 18.8, 14.07, 14.06.

### Synthesis of RCB-02-104-4

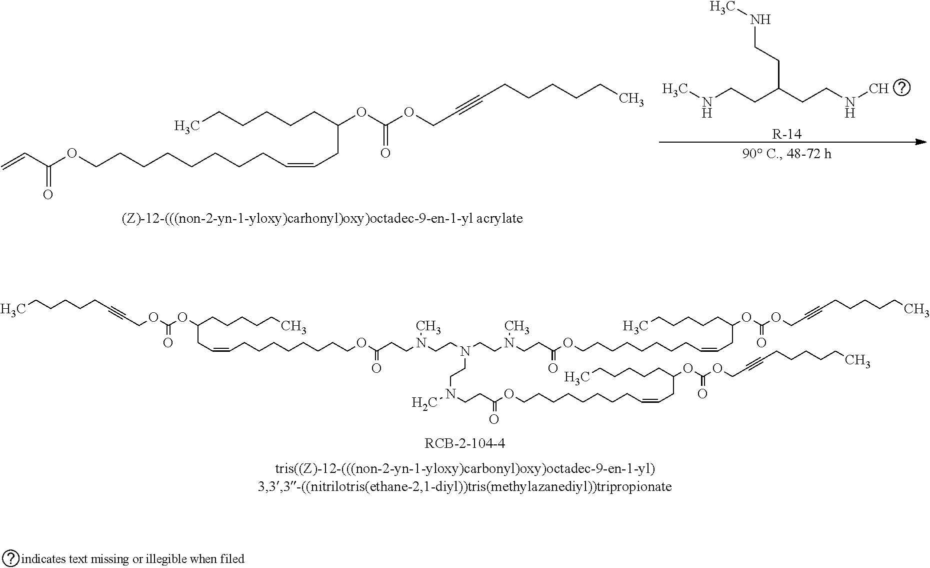

Into a 1-dram scintillation vial equipped with magnetic stir bar was placed (Z)-12-(((non-2-yn-1-yloxy)octadiccarbonyl)oxy)octadec-9-en-1-yl acrylate (254 mg, 0.5 mmol, 2.5 equiv) and Tris[2-(methylamino)ethyl]amine (38 mg, 0.2 mmol, 1.0 equiv). The vial was sealed with a silicone-lined screwcap and stirred at 90° C. for 2-3 days or until complete conversion of the starting material amine. Further purification is achieved by flash column chromatography on an Isco Combiflash system eluting with a gradient (0-100%) Ultra (3% concentrated ammonium hydroxide, 22% methanol, 75% dichloromethane): dichloromethane to afford tris((Z)-12-(((non-2-yn-lyloxy)octadiccarbonyl)oxy)octadec-9-en-1-yl) 3,3′,3-((nitrilotris(ethane-2,1-diyl))tris(methylazanediyl))tripropionate as a colorless oil (Yield: 222 mg, 73%).

¹H NMR (500 MHz, CDCl₃) δ 5.54-5.47 (m, 3H), 5.37 (dtt, J1=10.7, J2=7.3, J3=1.6 Hz, 3H), 4.76-4.70 (m, 8H), 4.08 (t, J=6.8 Hz, 6H), 2.73 (t, J=7.4 Hz, 6H), 2.62 (dd, J1=9.0, J2=5.7 Hz, 6H), 2.48 (t, J=7.3 Hz, 10H), 2.44-2.30 (m, 7H), 2.23 (tt, J1=7.2, J2=2.3 Hz, 7H), 2.25-2.28 (bs, 6H), 2.10-1.99 (m, 6H), 1.76-1.57 (m, 17H), 1.52 (p, J=7.1 Hz, 7H), 1.42-1.24 (m, 71H), 0.98-0.81 (m, 18H).

¹³C NMR (126 MHz, CDCl₃) δ 172.7, 154.6, 133.1, 123.6, 88.4, 79.0, 73.5, 64.6, 56.0, 55.6, 53.33, 53.0, 42.4, 33.5, 32.5, 31.9, 31.7, 31.3, 29.5, 29.5, 29.3, 29.1, 28.7, 28.5, 28.35, 27.38, 25.9, 25.19, 22.6, 22.5, 18.8, 14.07, 14.06.

Ester-Derived Novel Biodegradable Ionizable Lipids for the Delivery of mRNA to the Liver and Lungs

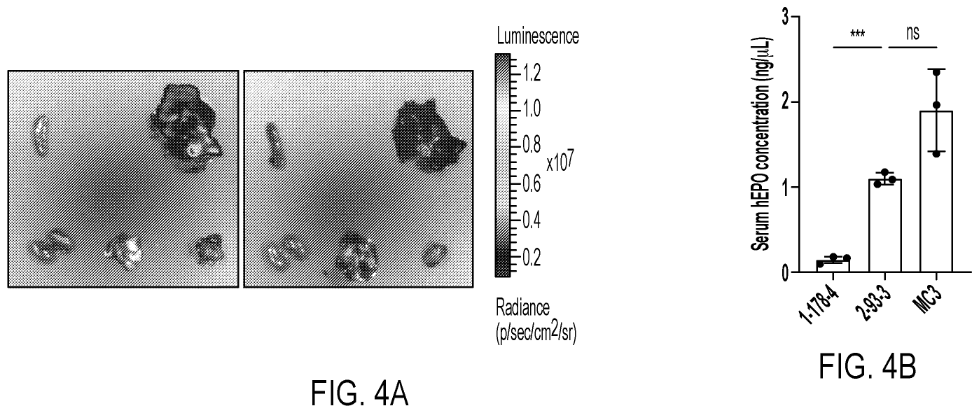

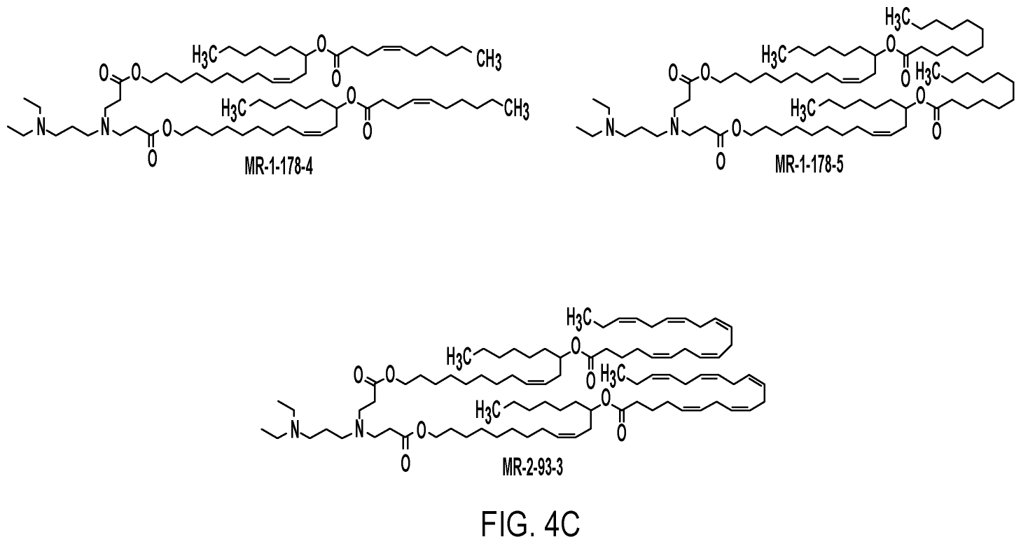

To determine whether any of the members of this library could be effective at targeted mRNA delivery in vivo, lipid nanoparticles were formulated using each of the synthesized lipids with DOPE, cholesterol, and C14-PEG2000 and evaluated their ability to deliver mRNA using a firefly luciferase (Fluc) reporter assay. This assay identified a number of lipids that were able to effectively deliver mRNA and effect Fluc protein expression selectively in the liver (FIG. 4A). Further optimization of the structure of MR-1-178-4 resulted in an order of magnitude increase in protein expression with MR-2-93-3, which results in a level of protein production comparable to Dlin-MC3-DMA, a benchmark lipid for nucleic acid delivery to the liver (FIG. 4B).

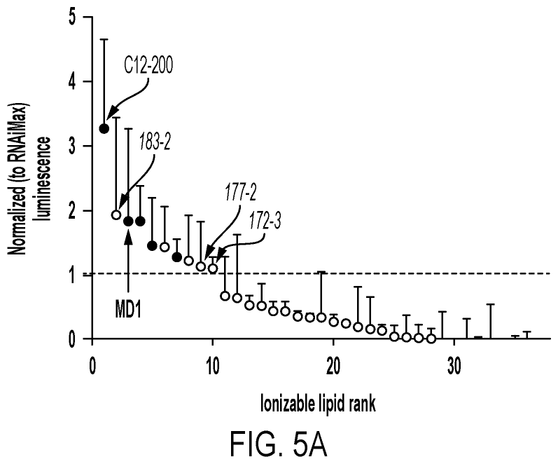

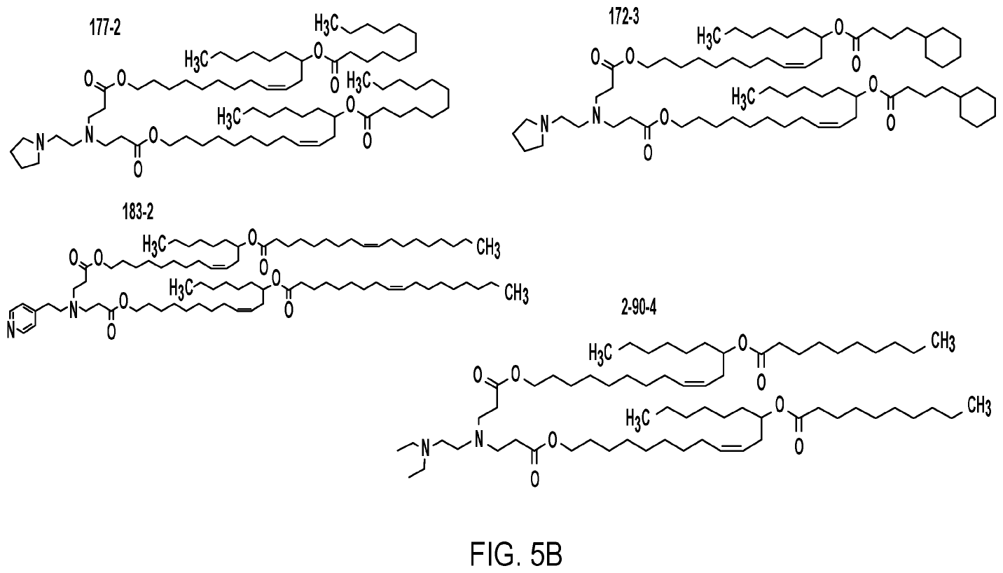

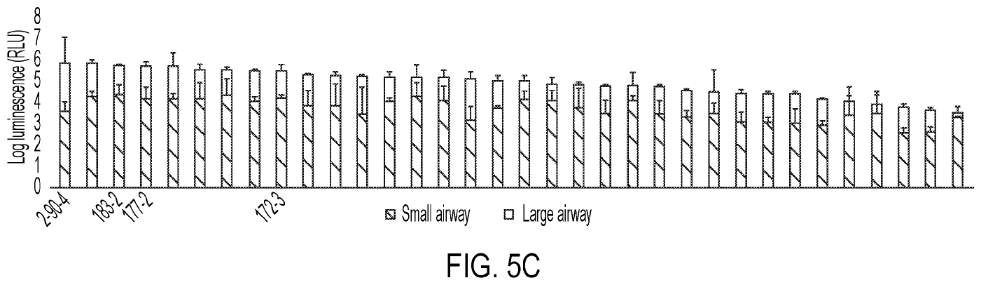

In order to identify lipids that could be used for nebulized delivery to the lung epithelium, mRNA delivery by LNPs was screened in vitro in A549 cells, identifying a number of potent in vitro LNPs. The top-performing lipids were screened for mRNA delivery in air-liquid interface (ALI) cultures. ALI cultures are a highly physiologically relevant in vitro model of the lung epithelium that recapitulates key lung properties and barriers to mRNA delivery, including mucus secretion, cell differentiation, and tight junction formation.⁷ Lipids were screened as part of LNPs containing 50.1% ionizable lipid, 24.6% DOTAP, 16.8% cholesterol, 8.5% PEG-lipid by mass, and a 10:1 ionizable lipid:mRNA weight ratio. LNPs generated using the MR-1-177-2 and MR-1-183-2 ionizable lipids performed excellently in ALI cultures (FIG. 5), along with numerous other lipids containing a headgroup matched to that of MR-1-177-2.

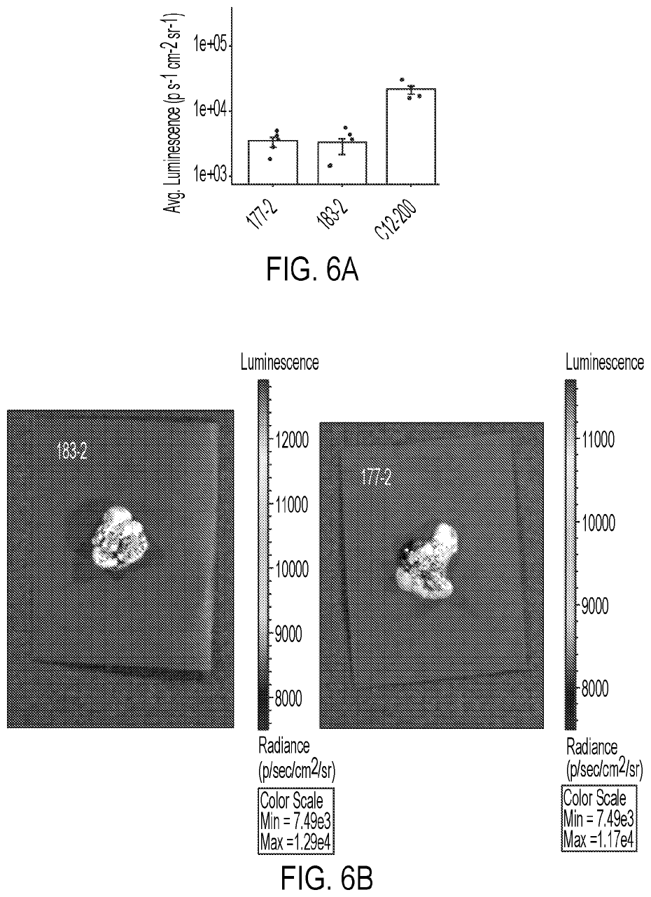

The two top candidates, MR-1-177-2 and MR-1-183-2, were tested in vivo via nebulization. They transfect the lung epithelium (FIG. 6) at a level 6.5-fold lower than C12-200, a promising level of transfection for biodegradable lipids.

The ability to efficiently deliver mRNA to the lung epithelium represents a significant advance in the treatment of lung diseases such as cystic fibrosis via RNA-based gene editing⁸ or via direct delivery of mRNA encoding therapeutic proteins.⁹ The use of biodegradable lipids, in particular, may allow for repeat dosing without toxic bioaccumulation of ionizable lipids.

Carbonate-Derived Novel Biodegradable Ionizable Branched Lipids for the Delivery of mRNA to the Lungs

Carbonate-derived ionizable lipids were synthesized by reacting ricinoleic acrylate or stearyl acrylate with 4-nitrophenyl chloroformate in the presence of pyridine followed by reacting with different alcohols (FIG. 7). Various alcohols such as saturated alcohols, alcohols having one or two double bonds, and alcohols with triple bonds were used.

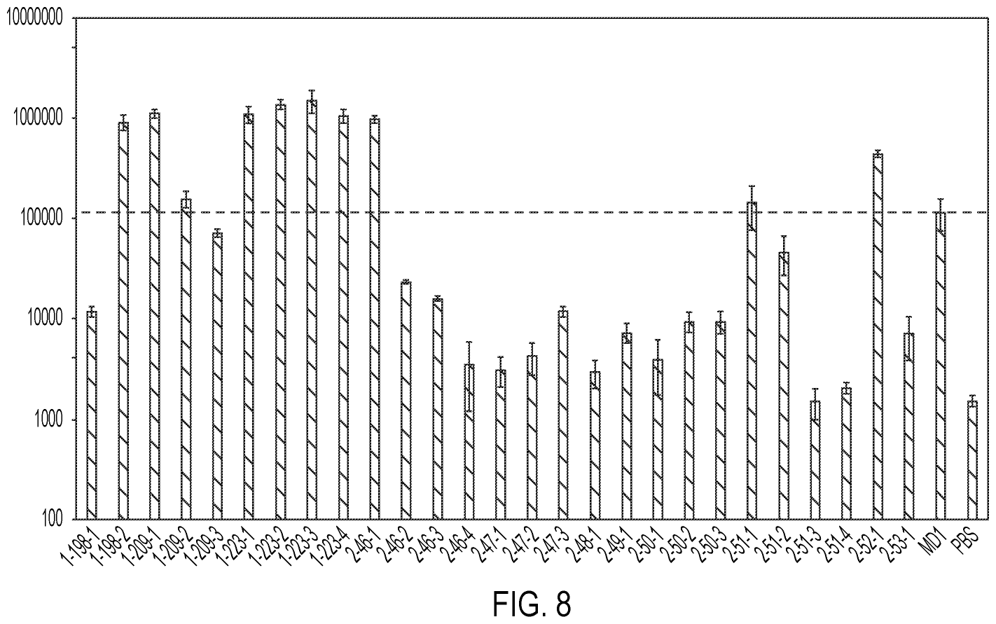

Around 30 lipids were synthesized and tested in vitro by using HEK293T cells (FIG. 8). Most of the carbonate-derived biodegradable lipids worked well, in fact, better than the control MD-1:

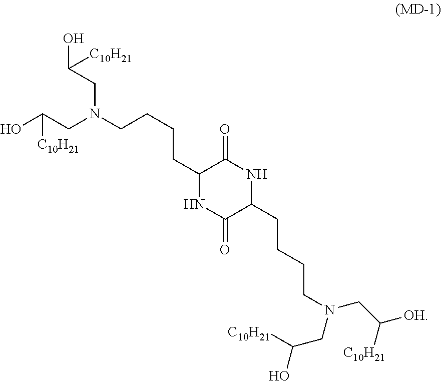

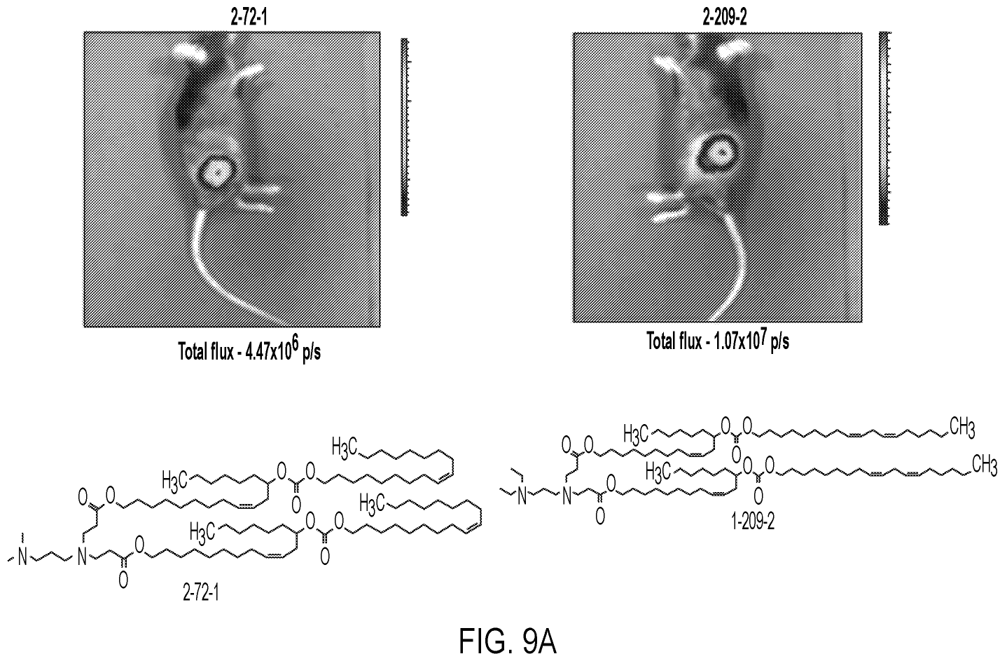

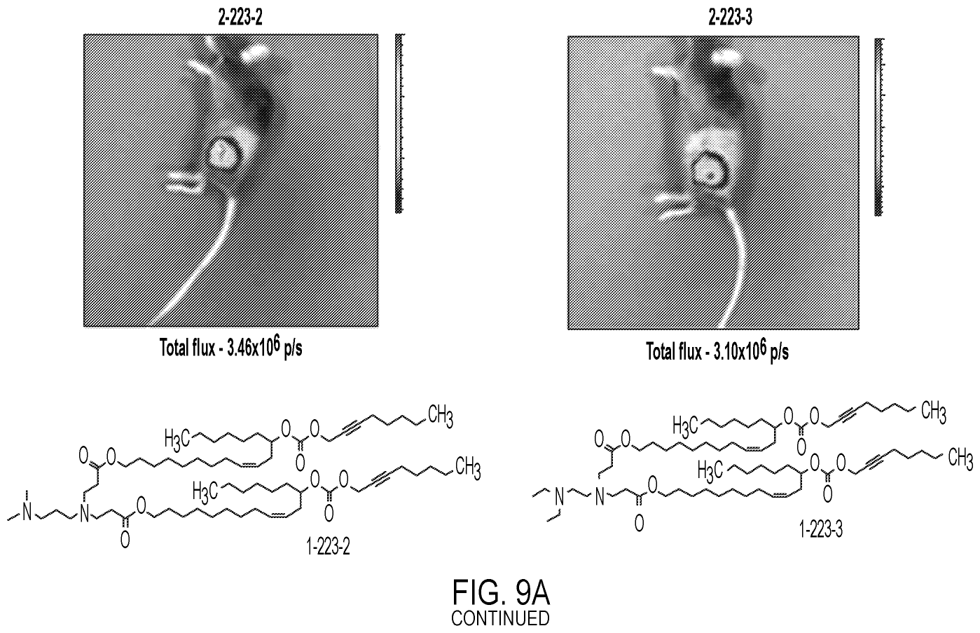

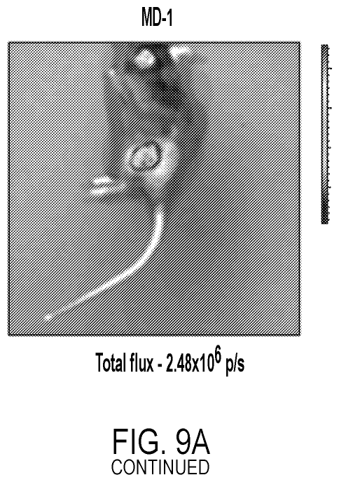

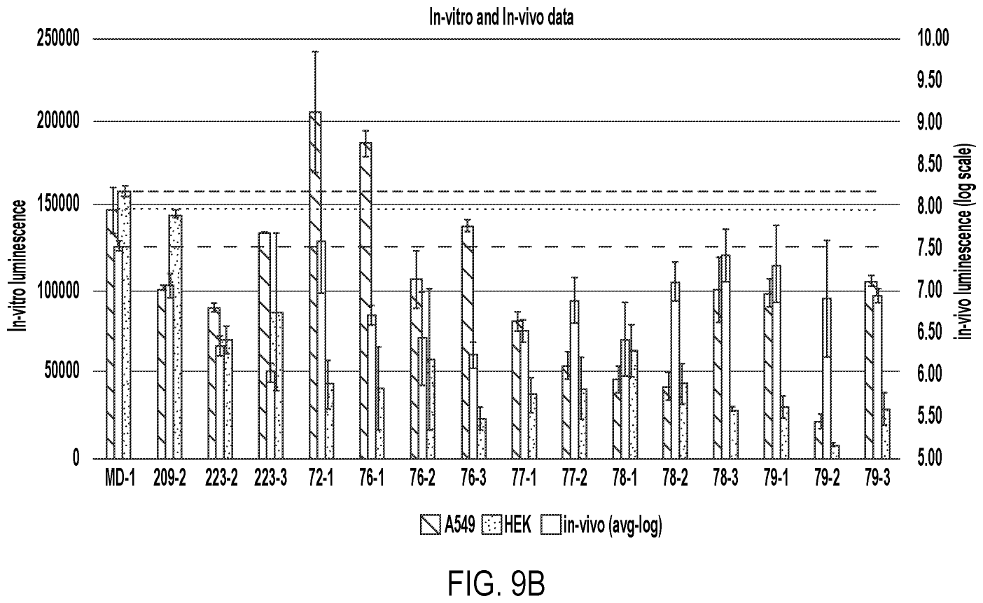

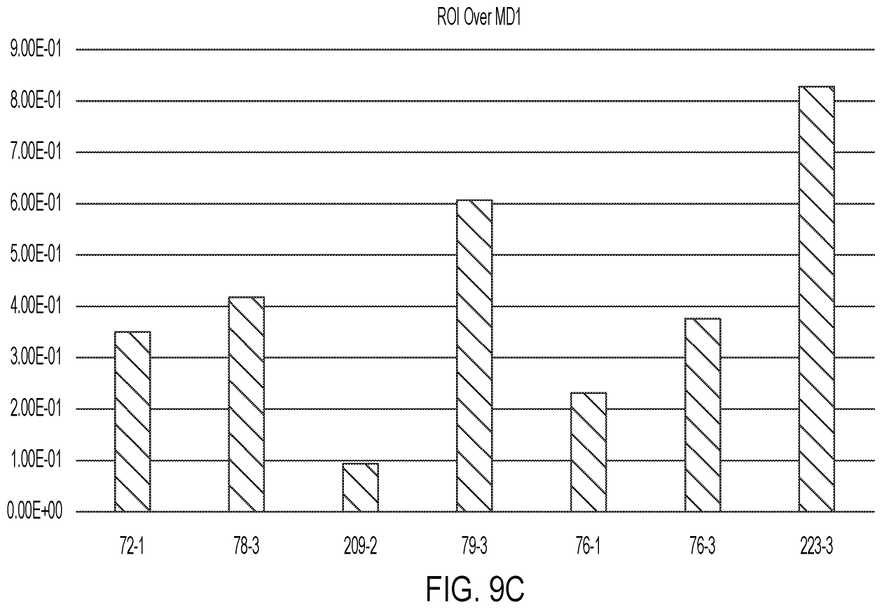

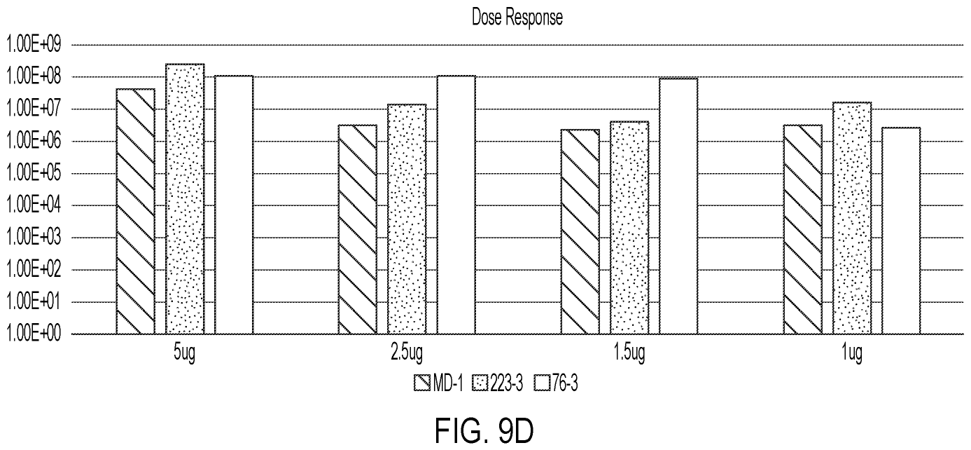

In vivo testing performed via intravenous injection did not show any transfection in any parts of the mice. Intramuscular and intratracheal administration was evaluated for mRNA delivery to the lungs. For the first round of batch of 28 lipids, four lipids showed much higher transfection when compared to control MD-1. Most of these top-performing lipids were found to be containing alkynes in between the tails. For A459 cells, two lipids showed higher transfection than MD-1. For HEK cells, none of the lipids showed higher transfection than MD-1. For IM cells, only one lipid that is RCB-2-72-1 showed improvement over MD-1 (FIG. 9). For IT administration, six lipids showed improvement relative to MD-1, and RCB-01-223-3 showed better transfection (FIG. 9C). Dose-response was performed for three lipids MD-1, RCB-01-223-3, and RCB-02-76-3 (FIG. 9D). At 5 μg, RCB-01-223-3 showed better transfection. At 2.5 μg and 1.5 μg, RCB-02-76-3 showed better transfection. At 1 μg, RCB-01-223-3 showed better transfection.

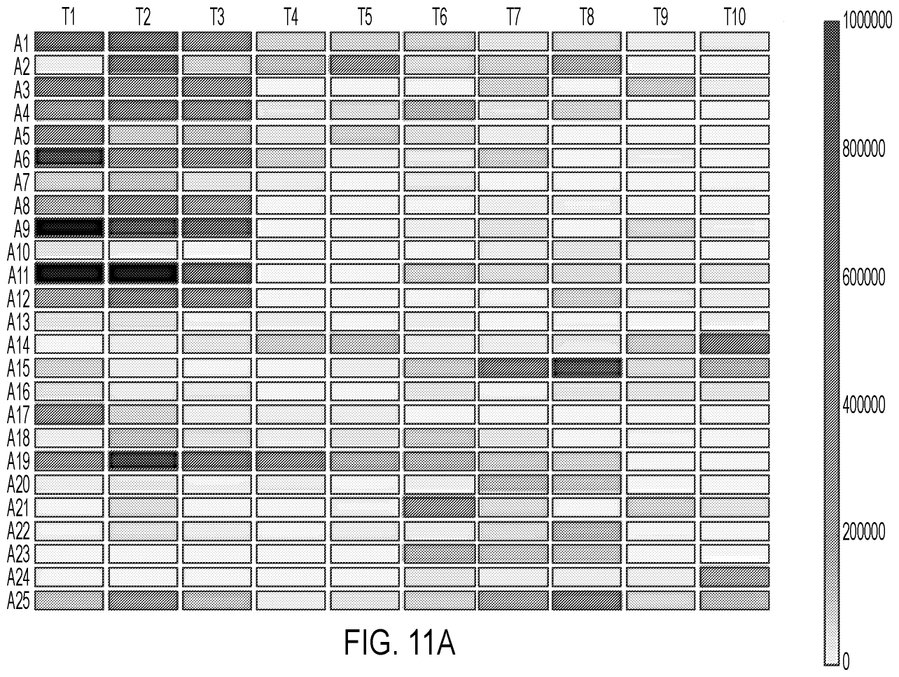

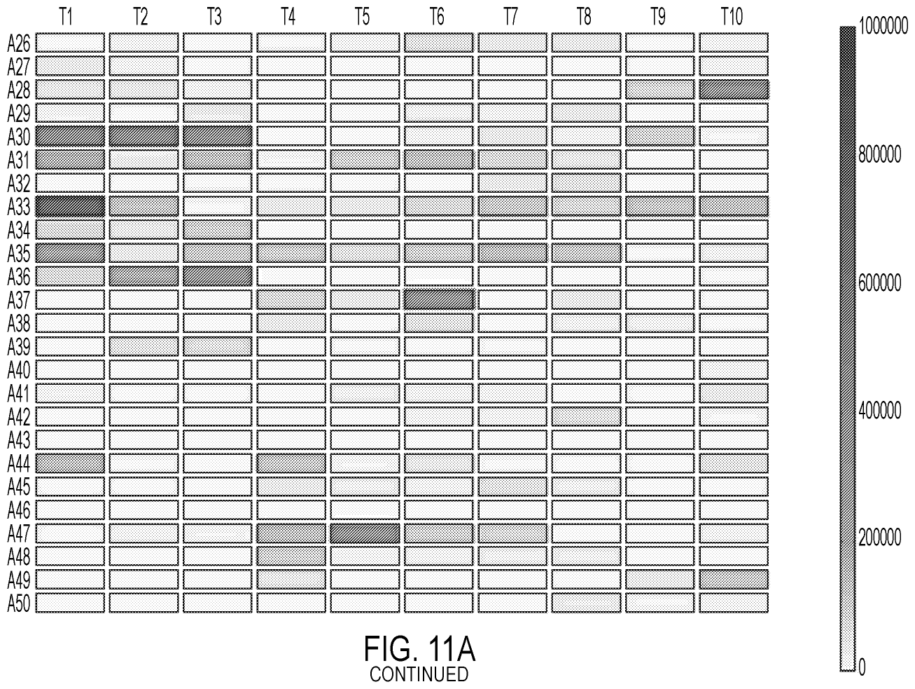

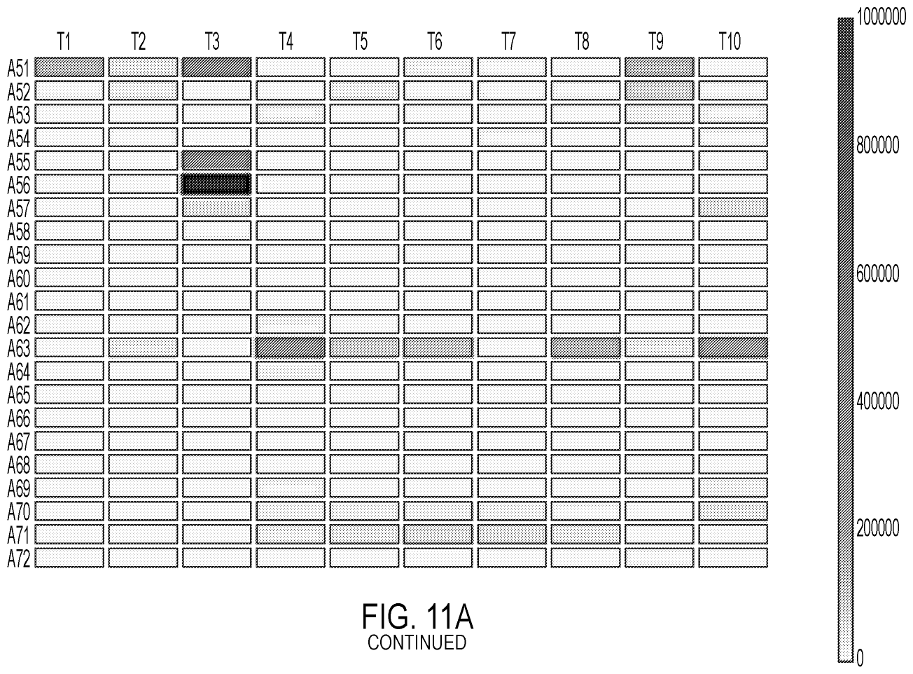

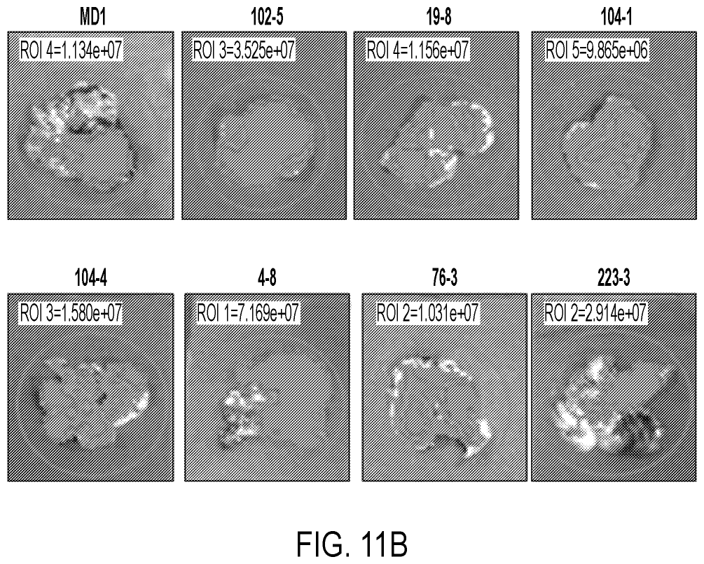

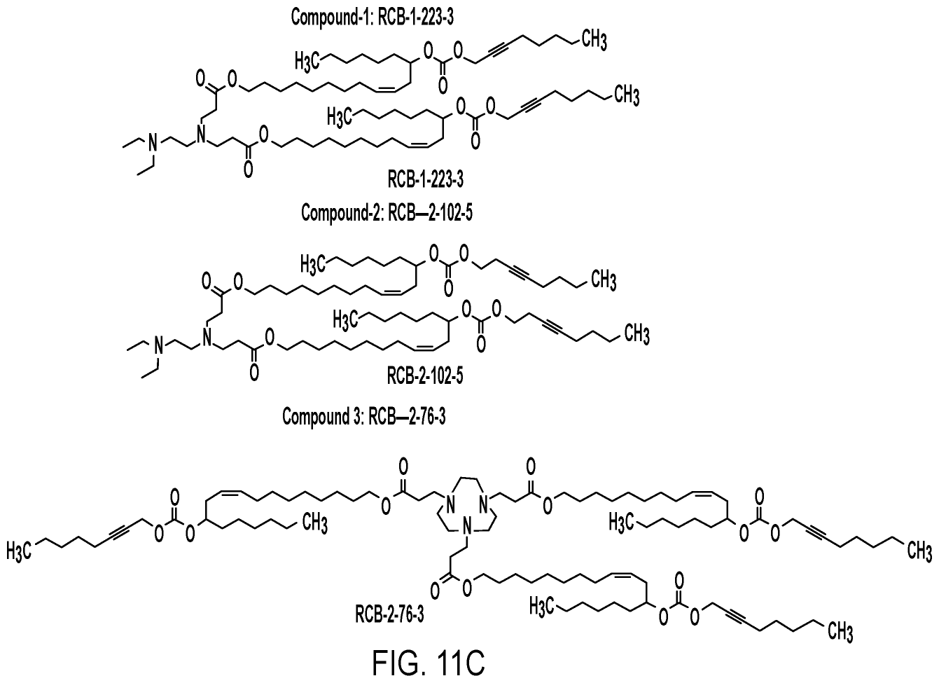

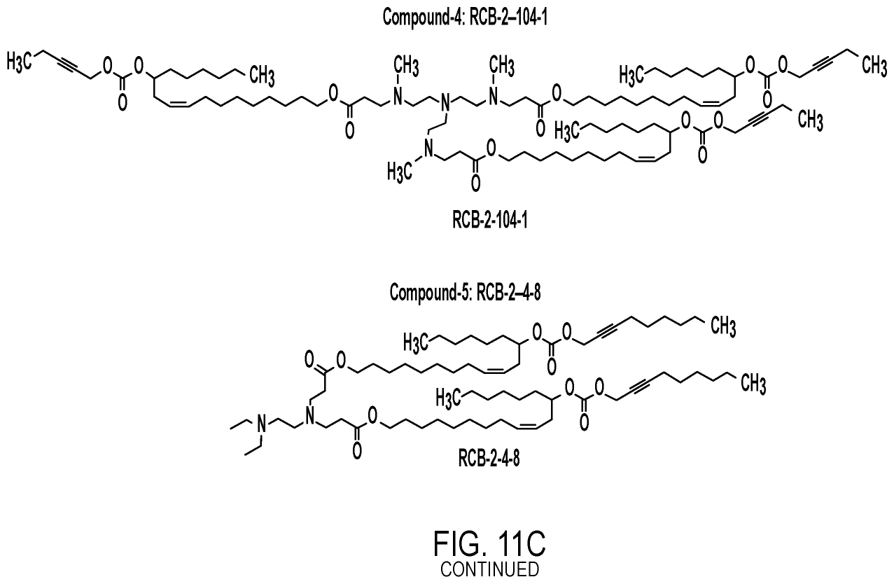

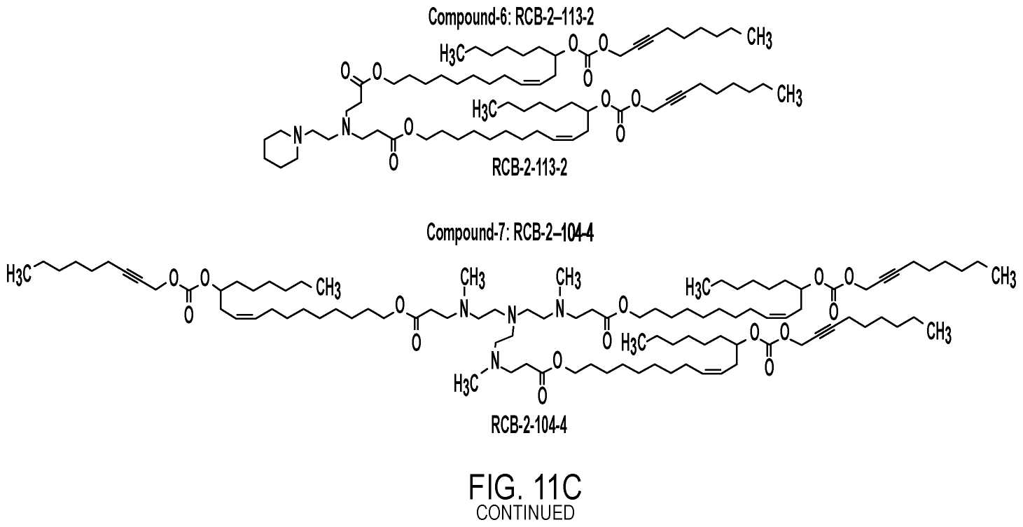

Inspired by these promising results, a combinatorial library of 720 biodegradable ionizable lipids was synthesized using the same chemistry. The library of amine groups and the lipids tails are shown in FIGS. 10A and 10B. In order to identify ionizable lipids that could be used for mRNA delivery to the lung, these lipids were formulated into LNPs loaded with firefly luciferase mRNA (mFFL). Their transfection performance was tested in A549 cells, identifying a number of potent lipids in vitro (FIG. 11A). The top-performing lipids from in vitro screening were evaluated for mRNA delivery in lung. Seven ionizable lipids (FIG. 11C) from this combinatorial library showed comparable or higher potency than MD-1 control in the lungs of mice post intratracheal (IT.) administration. Representative IVIS images are shown in FIG. 11B. The structures of the top-performing lipids are shown in FIG. 11C.

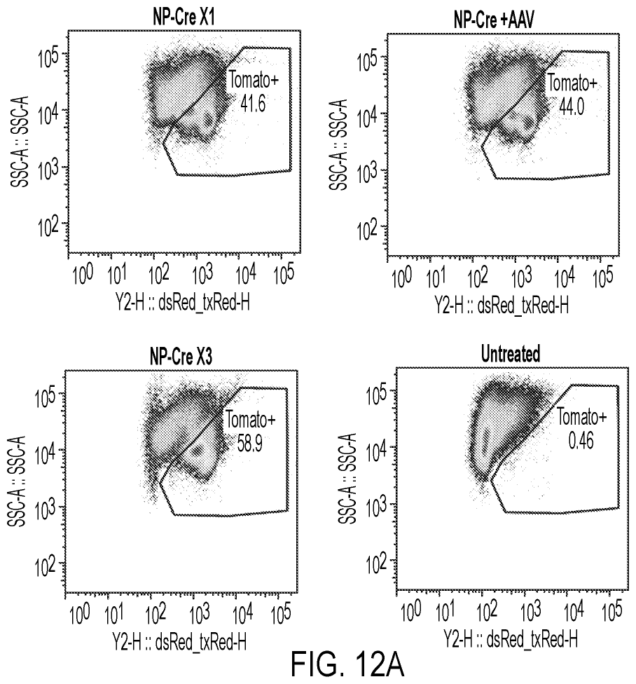

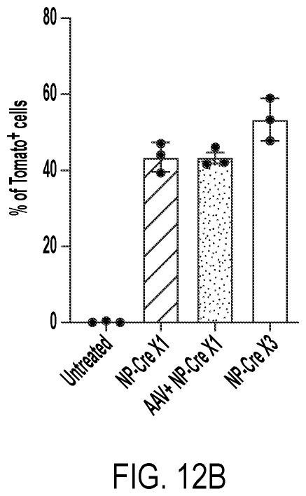

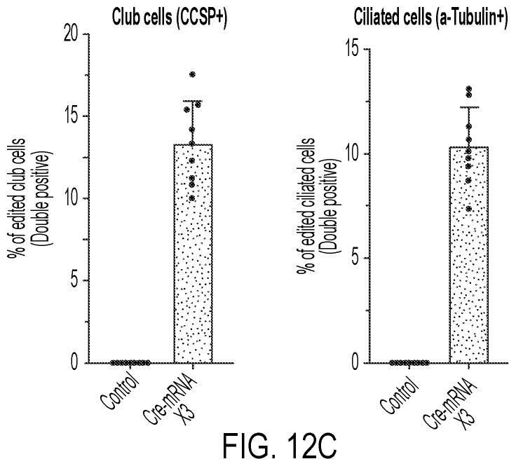

One of the top-performing lipids, lipid RCB-2-4-8, was formulated into LNPs for delivery of mRNA encoding Cre recombinase (mCre) to the lungs of Ai9 tdTomato reporter mice. These mice harbor a loxP-flanked stop cassette that controls gene expression of the fluorescent tdTomato protein which is only produced in the presence of Cre recombinase. Two cohorts of mice (n=3) were given one dose (day 0) and three doses (day 0, 2, 4) of mCre LNPs via I.T. administration, respectively. To test the potential immunogenicity of adeno-associated virus (AAV) on LNP, a third cohort of mice (n=3) was administered AAV via I.T. administration before the administration of mCre LNPs. The same dosage of mRNA (0.75 mg/kg) was adopted for all three cohorts of Ai9 mice. The lung cells were analyzed by flow cytometry (FIG. 12B). Results indicate that one-dose and three-dose I.T. administration led to successful transfection in ˜42% and ˜59% lung cells respectively. Meanwhile, pre-dosing of AAV did not affect the transfection potency of mCre LNP as ˜40% lung cells still expressed strong tdTomato fluorescence, similar to the result of single I.T. administration of mCre LNPs.

To further validate Cre-mRNA results, immunohistochemistry staining was performed on lung tissue sections. Antibodies against tdTomato and CCSP or acetylated-tubulin were used to detect edited cells (double-positive) in paraffin-fixed lung sections. 13±3% and 11±2% double-positive club and ciliated cells, respectively, were observed in the large airways under both confocal and standard fluorescence microscope (FIG. 12C). Together, these data demonstrate that the RCB-2-4-8 LNP can deliver mRNA to various cell types in respiratory epithelium and induce potent expression of functional proteins in these cells, providing a new avenue for using RNA therapeutics to treat lung diseases.

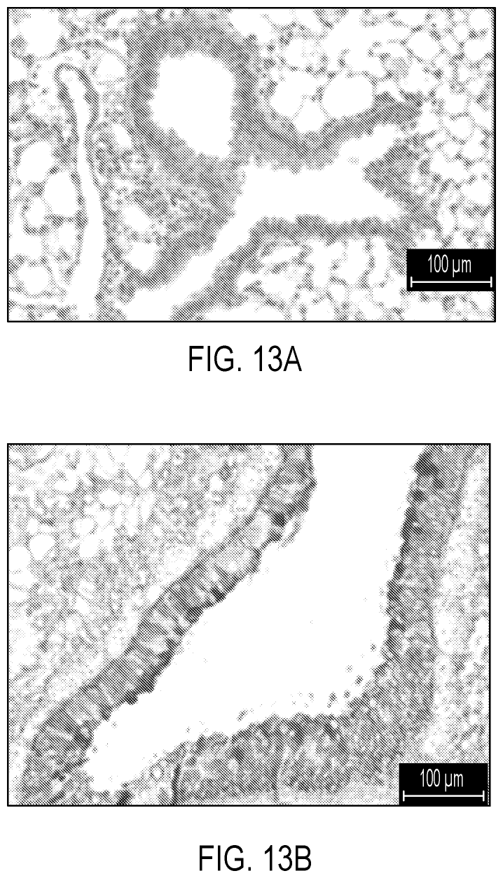

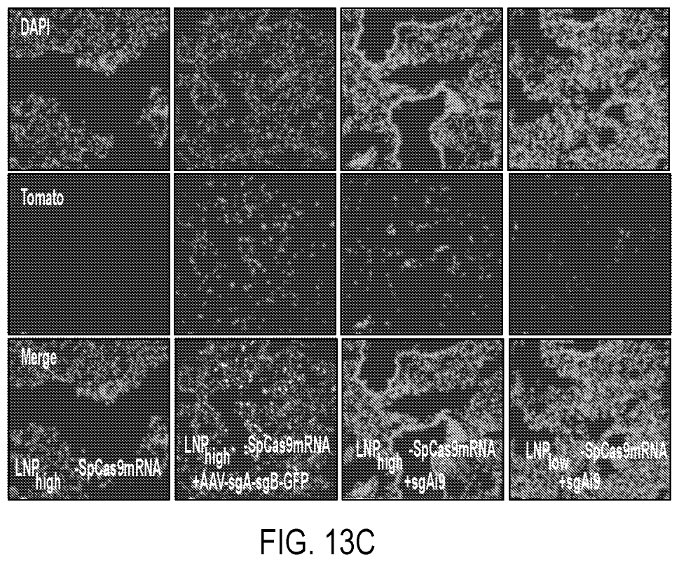

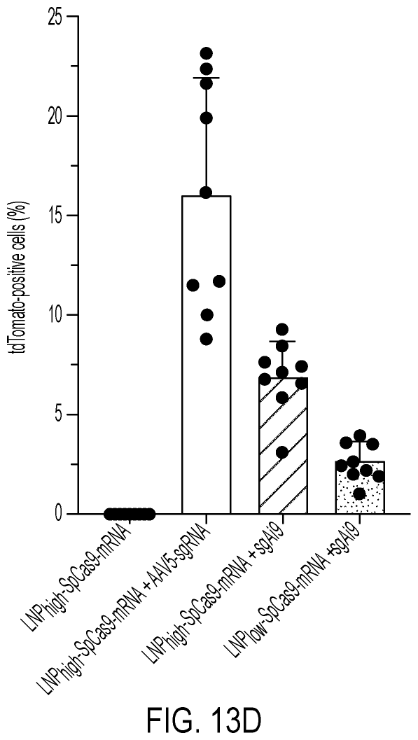

To reveal the potential of this LNP delivery system in gene editing, lipid RCB-2-4-8 was formulated into LNPs that contain a CRISPR-Cas9 mRNA (mCas9) and a loxP-targeting sgRNA. Three doses mCas9/sgRNA LNPs were given to Ai9 mice on day 0, 2 and 4 by I.T. administration, after which the mouse lungs were collected for immunostaining and histology analysis. Compared to the lung cells from untreated Ai9 mice (FIG. 13A), a high percentage of lung cells showed strong signal of tdTomato (darkest regions, FIG. 13B) after the I.T. administration of mCas9/sgRNA LNPs, demonstrating the ionizable lipid RCB-2-4-8 as an efficient tool for gene editing.

RCB-2-4-8 lipid was also formulated with CRISPR-Cas9 mRNA and a loxP-targeting sgRNA and administered intratracheally at low (LNP_{low}-SpCas9 mRNA+sgAi9) or high (LNP_{high}-SpCas9 mRNA+sgAi9) dose (0.5 or 1 mg·kg¹ total RNA) at 2-day intervals (three doses) and quantified gene editing on the 7^{th} day. In the absence of sgRNA, the negative control, RCB-4-8 LNPs carrying SpCas9 mRNA only (LNP_{high}-SpCas9 mRNA) did not display any tdTomato signal in the lung tissue; in contrast, LNP_{low}-SpCas9 mRNA+sgAi9 and LNP_{high}-SpCas9 mRNA+sgAi9 could result in 3+1.0% or 7±3.0% tdTomato-positive cells, respectively (FIGS. 13C and 13D).

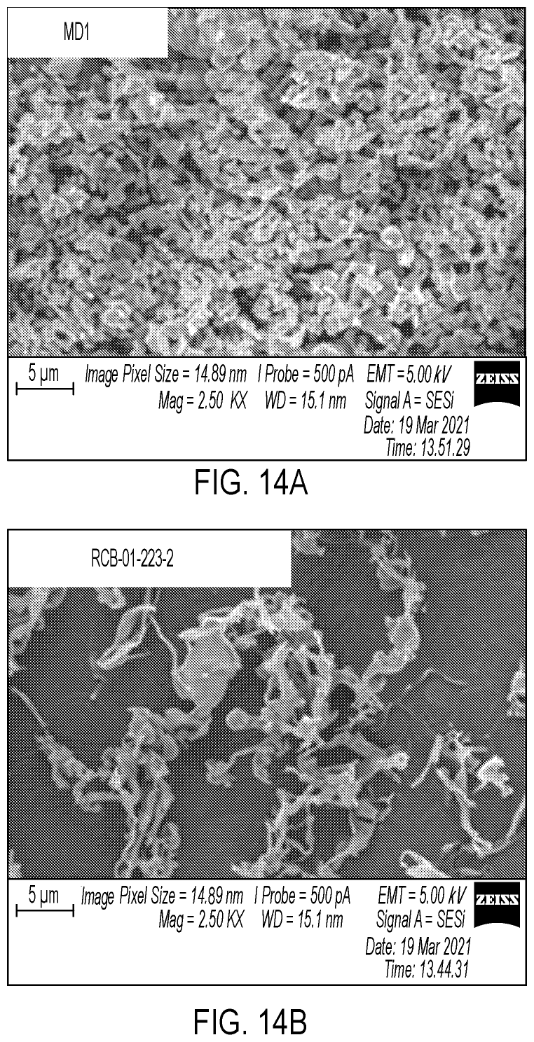

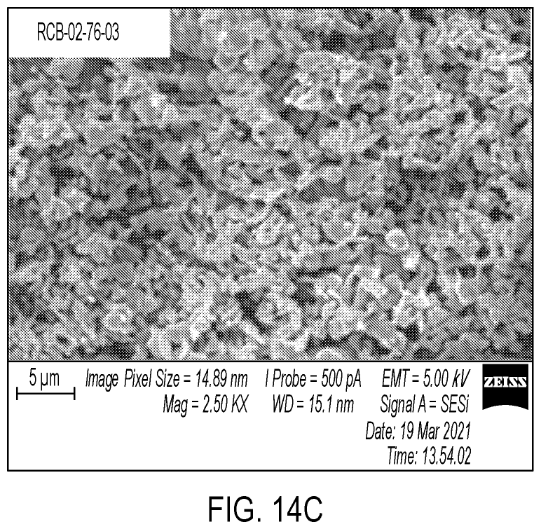

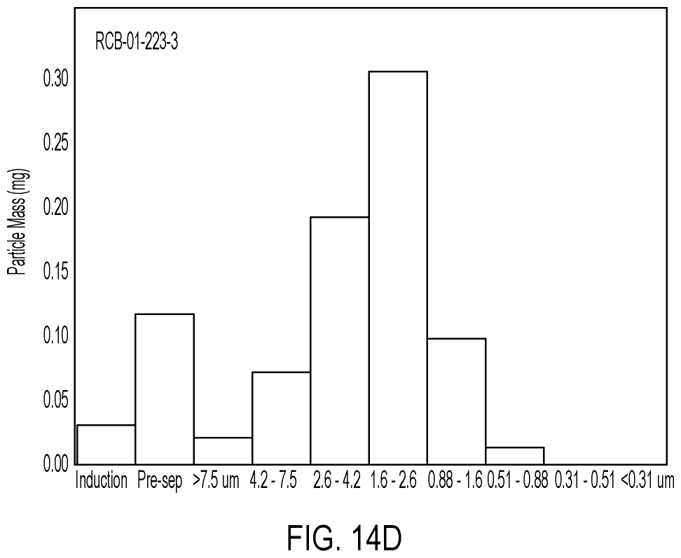

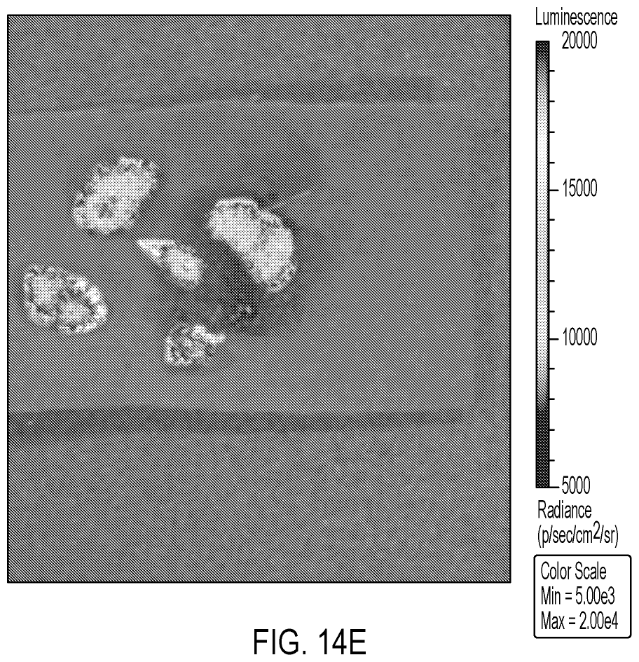

A technique to prepare an inhalable mRNA dry powder was also developed and optimized. Zhao, and Ball et al. published reported RNA dry powders that could be reconstituted in water for intravenous or intramuscular injection, but which are not optimized for direct delivery.¹¹ Dry powders for inhalation are generally produced via either spray drying or spray freeze drying using mannitol as the cryoprotectant and bulking agent.¹² Particles from the spray freeze-drying method are irregularly shaped with a physical diameter of 3 to 10 microns and a functional aerodynamic diameter of 0.9 to 7.5 microns (FIGS. 14A-14C). MMAD data showing aerodynamic size is shown in FIG. 14D. The dry powder showed both excellent lung distribution and effective local transfection from the preliminary analysis of dry powder administration to mice and rats (FIG. 14E).

### Carbamate-Derived Novel Biodegradable Ionizable Branched Lipids

Carbamate-derived ionizable lipids are synthesized from by reacting ricinoleic acrylate/stearyl acrylate with 4-nitrophenyl chloroformate in the presence of DMAP and DIPEA at 22° C. for 24 hours, followed by reaction with a primary amine in the presence of pyridine and DMAP at 22° C. for 1 hour (FIG. 2).

### Example 2

### Description Summary

The recent COVID-19 vaccine roll-out has brought to light two key facts: mRNA-based therapy is an effective and flexible tool to address public health measures, and the current cold-storage requirements for mRNA nanoparticles in solution are often prohibitively expensive. Therefore, there is a clear unmet need for shelf-stable mRNA products.

A flexible dry powder formulation for mRNA-containing nanoparticles has been developed. Dry powders can be easily self-administered by patients via established inhaler technologies (popularized by many asthma therapies such as the Advair Diskus), and boast shelf lives of months to years blister-packed at ambient conditions. They also facilitate drug delivery to the lungs, enabling novel treatment options for conditions such as cystic fibrosis.

Dry powders of four different ionizable lipid carriers that encapsulate two different mRNA sequences have been produced using a spray freeze drying (SFD) method via the inclusion of mannitol as a cryo-protectant. All powders retain mRNA biological activity following the SFD process, suggesting that a much wider variety of lipids and mRNA could be easily substituted without the need to significantly vary process parameters. Powder size and density have been optimized for deep lung deposition. Initial results have shown promising size distribution and excellent powder distribution and mRNA activity in mouse and rat models. A flexible new process has been developed to produce stable and effective mRNA-containing dry powders, enabling a wide variety of further medical applications.

Design and Optimization of a Dry Powder for Stable Storage and Pulmonary Delivery of mRNA

RNA therapeutics, utilizing lipid nanoparticles encapsulating messenger or silencing RNA, represent a new and growing class of pharmaceuticals. However, there are significant ongoing concerns about particle stability and long-term storage, several of which have received high visibility in the COVID-19 vaccine roll-out. To address this limitation, novel optimized spray freeze-drying technique for the production of inhalable RNA dry powders have been developed. These powders retain up to 100% of expected biological activity following reconstitution, varying with lipid composition, and are stable at 4° C. under dry conditions for at least 90 days. The powder microparticles are irregularly shaped with an observed diameter of 4-10 microns and are well suited to deposition in the deep lung. Two mRNA sequences have been tested with both showing transfection in-vivo following direct dry powder administration to rodent models.

### Introduction

An inhalable mRNA dry powder has been developed and optimized. Although a few groups have recently published on RNA powders, these are not optimized for direct delivery and must instead be reconstituted in water for intravenous or intramuscular injection.^{11,20} This reconstitution generally would require a sterile solution in a clinical context as well as injection by a trained professional, greatly limiting their potential for global use and patient self-administration. In contrast, dry powder inhalers are a well-established delivery mechanism for pulmonary drugs, and have recently seen wide usage in asthma medications such as the Advair Diskus. These dry powders are easy for patients to self-administer and are usually very stable, lasting for weeks to months when blister packed at ambient conditions.¹² Dry powders for inhalation are generally produced via either spray drying or spray freeze drying.¹² Due to the reduced stability of mRNA lipoplexes at higher temperatures,¹¹ spray freeze drying has been pursued.

To achieve deposition in the deep lung, it is necessary for a powder to have an aerodynamic diameter between 1 and 5 microns, as smaller particles are rapidly exhaled and larger particles settle in the mouth and trachea. It is notable, however, that the observed aerodynamic diameter can be somewhat smaller than the physical diameter of the particle, as less dense particles move more easily through the airways.²¹ A spray-freeze-drying process has been developed in which mannitol is used as a cryoprotectant and bulking agent. The particles created are irregularly shaped with an observed physical diameter between 3 and 10 microns, and a functional aerodynamic diameter of 0.9 to 7.5 microns. In preliminary testing of direct powder administration to mice and rats, the powders show both excellent lung distribution and significant local transfection, and are stable at room temperature under desiccating conditions.

The dry powders described herein are useful in a variety of situations, including as inhaled vaccines for infectious diseases or cancer and as targeted local therapeutics for conditions such as cystic fibrosis or asthma.

### Methods

### Spray Freeze Drying (SFD):

mRNA-containing lipid nanoparticles were prepared with additional cryoprotectants in the aqueous phase to a final concentration of 200 μg mRNA/ml. Particles were then diluted to a final nominal mRNA concentration of 25 μg/ml (8-fold dilution) in Rnase-free water. The solution was then fed through the peristaltic pump of a Buchi Mini Spray Dryer 290 with a pump setting of 30% (440 mL/hour). The solution was collected in liquid nitrogen at the atomizer outlet of the spray dryer with a nozzle air flow rate setting of 30 mm corresponding to a nominal flow rate of 357 L/hour. The collected particles were then dried using a pre-cooled shelf lyophilizer at −20° C. for at least 24 hours (up to 72 hours).

Physical and aerodynamic size of particles was assessed using a Sympatec Helos particle size analyzer and MSP NGI cascade impactor respectively. For cascade impactor measurements, modified particles with an additional encapsulated fluorescent dye (DiL) were used and quantification of mass fractions was carried out via fluorescence (excitation 530 nm, emission 570 nm). Particle morphology was further analyzed via scanning electron microscopy (SEM) imaging on a Zeiss Crossbeam 540 SEM/FIB at room temperature following gold thin film coating.

### In-Vitro and In-Vivo Dosing of SFD Dry Powders:

SFD particles were dissolved in Rnase-free water at room temperature at approximate concentrations between 1 and 10 mg/ml. Exact mRNA concentration was determined via the Invitrogen Quant-iT Ribogreen assay (carried out as per the manufacturer's protocol), and used to normalize dosing prior to in-vitro testing. Ribogreen was not used to confirm dosing in-vivo, as the powder was dosed directly and the mRNA dosage was estimated based on the total mass of powder delivered instead. In-vivo dosing was carried out via direct pulmonary insufflation of the dry powder using a tracheal cannula loaded with the powder and a syringe. In-vitro results were quantified using the Promega BrightGlo reagent, and in-vivo results were quantified using an In-Vivo Imaging System (IVIS) following luciferin injection (in the case of luciferase transfection) and lung extraction.

### Results

A Stable mRNA Dry Powder Retaining Biological Activity was Developed

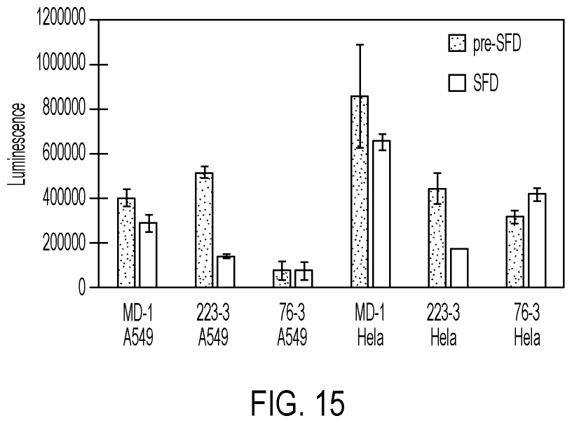

Dry powders were prepared as described above for three proprietary lipids (MD-1, RCB-01-223-3 and RCB-02-76-3) using firefly luciferase mRNA. Each powder was dosed to A549 and HeLa cells at 60 ng/well, with a cell density of approximately 30,000 cells/well (FIG. 15). Across the cell lines observed, MD-1 particles retained 72-76% of original activity, RCB-01-223-3 particles retained 27-39% of original activity, and RCB-02-76-3 particles retained over 96% of original activity. Solubility of RCB-02-76-3 particles was observed as a confounding factor in the accurate determination of in-vitro dosage, but these data demonstrate that a powdered form of mRNA LNPs retains relevant biological activity.

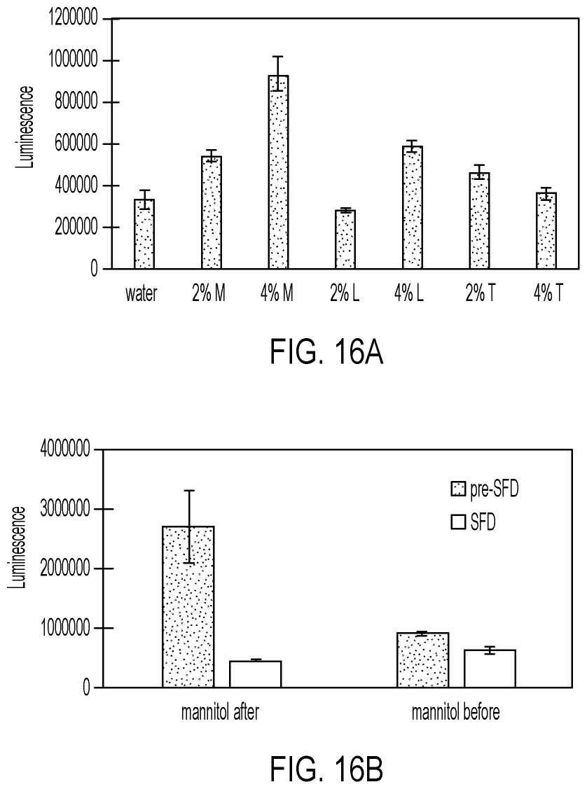

Additional particles were prepared containing a variety of cryoprotectants, to confirm the optimal effects of mannitol. As can be seen in FIG. 16A, mannitol outperformed all other cryoprotectants tested. Additionally, the observed particle size of the powders prepared with other cryoprotectants was significantly larger than that of those prepared with mannitol (most notably for lactose, whose particles were over 40 microns in diameter). Although a higher percentage of mannitol led to greater retained activity, mannitol's role as a bulking excipient limits the total powder that can be easily administered, requiring the dosing of more powder despite the higher activity. Additionally, the need to include mannitol at the formulation step, rather than being added prior to the SFD process was confirmed via the production of two otherwise-identical particles prepared with mannitol in formulation and with mannitol added afterward to the same final concentration. Both particles were made into powders and their transfection assessed in-vitro (see FIG. 16B). Although the inclusion of mannitol at formulation has a significant detrimental effect on particle transfection, the powder produced has greater relative and absolute potency.

A Powder with Desirable Characteristics for Lung Deposition was Optimized

Using the dry powder preparation and characterization described above via, powders were found to have physical diameter between 3 and 10 microns with low density, which is favorable for deep lung deposition. SEM imaging of the powders revealed that they were not spherical, but rather irregularly shaped (FIGS. 14A-14C). Cascade impactor measurements showed that the aerodynamic size distribution for RCB-01-223-3 particles contained a small population of oversized particles with the rest normally distributed around 3 microns, with approximately 70% falling between 1 and 5 microns (FIG. 14D). It is expected that particles with these size characteristics would deposit well in the deep lung in humans or animal models.

### The Powder Showed Potent and Well-Distributed Activity in Animal Models

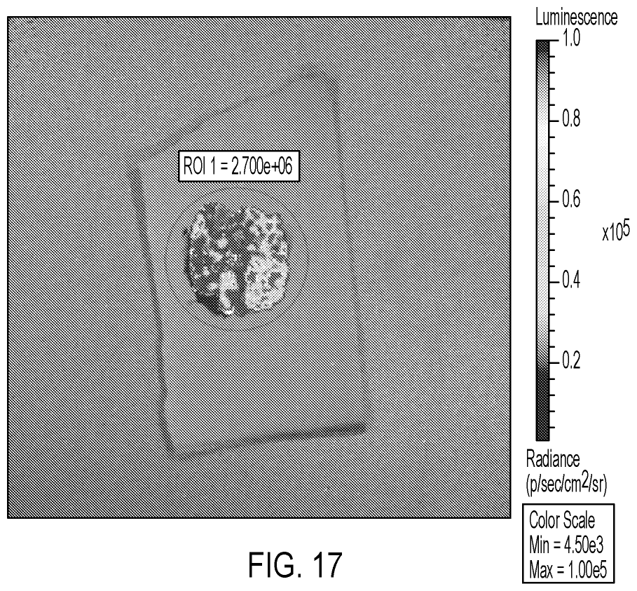

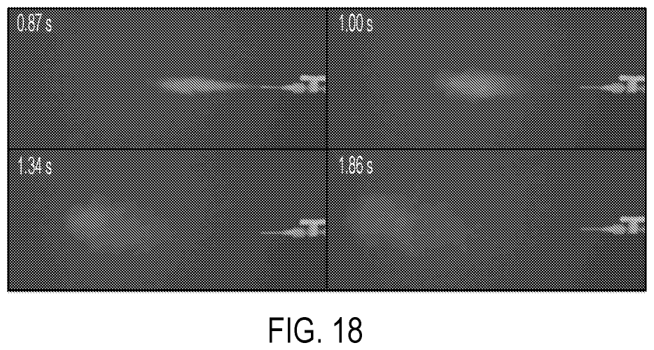

Following the promising results noted above in diagnostic assays and in-vitro, the powder was used to transfect lung cells in rat and mouse models. Using firefly luciferase mRNA, good distribution of the powder in the mouse lung with uniform transfection in all five lobes was observed (FIG. 14E, FIG. 17). This data is corroborated by the excellent aerosol characteristics observed in slow-motion video of the insufflator (FIG. 18). Similarly, in rat models, good distribution of the powder was observed. The dry powder afforded greatly improved distribution and stability.

### Discussion

As hundreds of millions around the globe receive novel mRNA vaccines against CoVID-19, and clinical trials for dozens of mRNA therapeutics are ongoing, it is increasingly important to have stable and easily deliverable modalities for mRNA therapeutics. Unfortunately, current mRNA modalities are generally liquids, requiring −20° or even −80° C. storage beyond a few days and intramuscular or intravenous injection by a trained professional. Development of a stable and effective dry powder modality for mRNA that can be easily self-administered by patients is a significant step toward more widely-usable and less-expensive mRNA therapeutics.

Various powder compositions, including different nanoparticle compositions, cryoprotectants and spray freeze drying conditions have been explored to improve the retained mRNA activity and to optimize powder size for efficient lung delivery. The optimized powder and spray drying conditions consistently yield powders with narrow size distributions at a desirable diameter for lung distribution and with significant biological activity. These powders are also significantly more stable under desiccating conditions at room temperature than nanoparticles in solution. In testing, the particles have shown good size distribution in a cascade impactor lung model and excellent deposition in-vivo in rodent models.

### Example 3

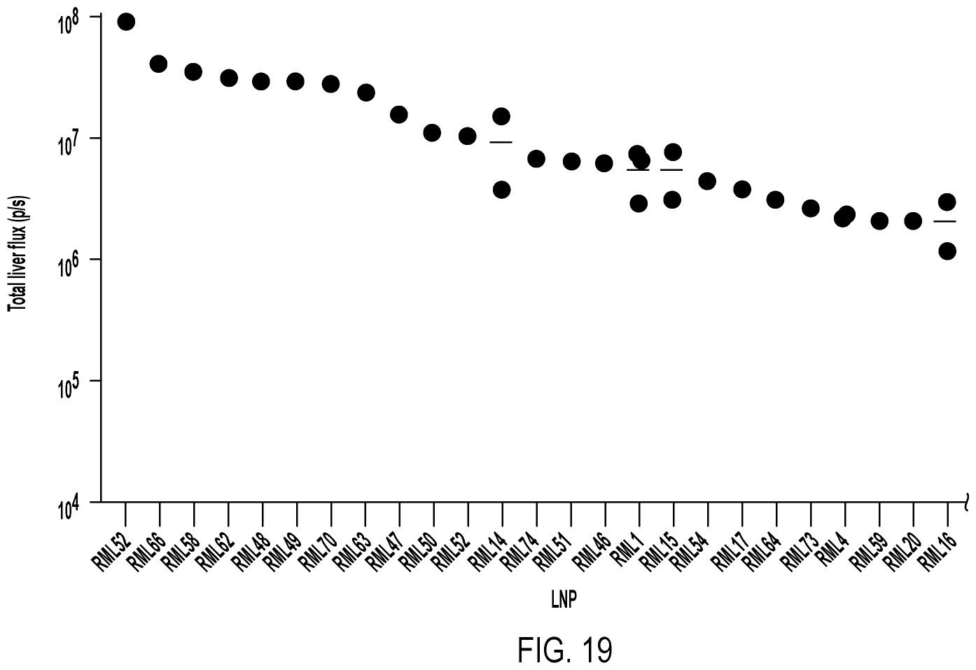

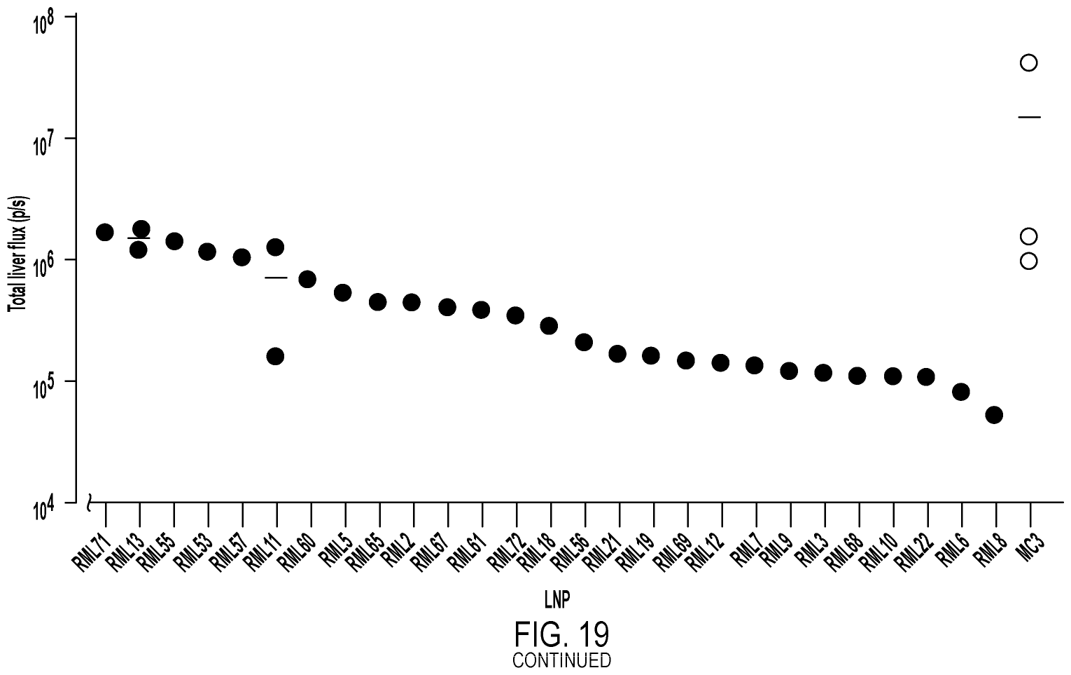

In order to identify lipids that could be used for liver mRNA delivery, 1941 LNPs were screened in silico using a deep learning model. LNPs were selected for testing based on the deep learning results. The top predicted lipids were tested for liver mRNA delivery intravenous (IV) delivery of 1p g total firefly luciferase (FFL) mRNA (approximately 0.05 mg/kg). The lipids were formulated using a formulation of 72.3% ionizable lipid, 7.8% DOPE, 15.6% cholesterol, and 4.2% PEG-lipid by mass (not molar ratios) and (unless otherwise specified) a 20:1 ionizable lipid:mRNA weight ratio. Promising lipids included RML52, RML58, and RML66 (FIG. 19).

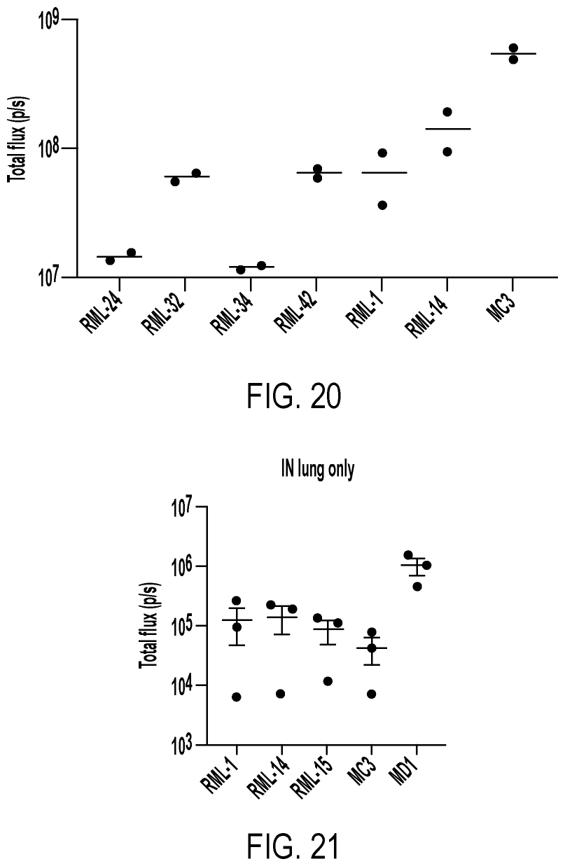

Testing LNPs with amino-alcohol headgroups combined with top tails predicted by machine learning afforded good intramuscular mRNA delivery activity (FIG. 20).

Lipids RML-1 to RML-22 were tested for intranasal (IN) administration. In lungs, RML-1 and 14 exhibit almost 3.5 times higher transfection than MC-3 and 7 times lower than the MD-1 (FIG. 21). In this test, the lipids were formulated using a formulation of 50.0% ionizable lipid, 10.0% DOPE, 38.5% cholesterol, and 1.5% PEG-lipid by mass and a 20:1 ionizable lipid:mRNA weight ratio.

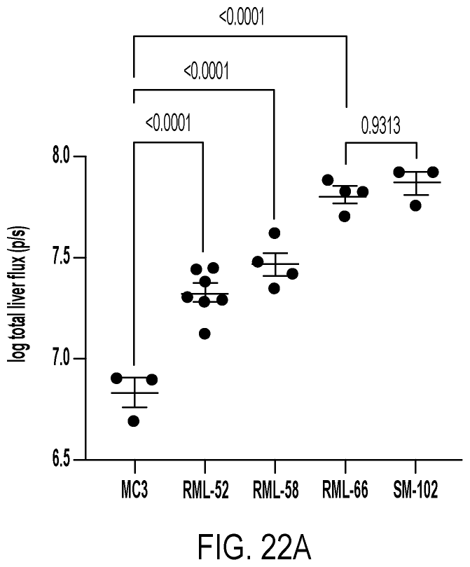

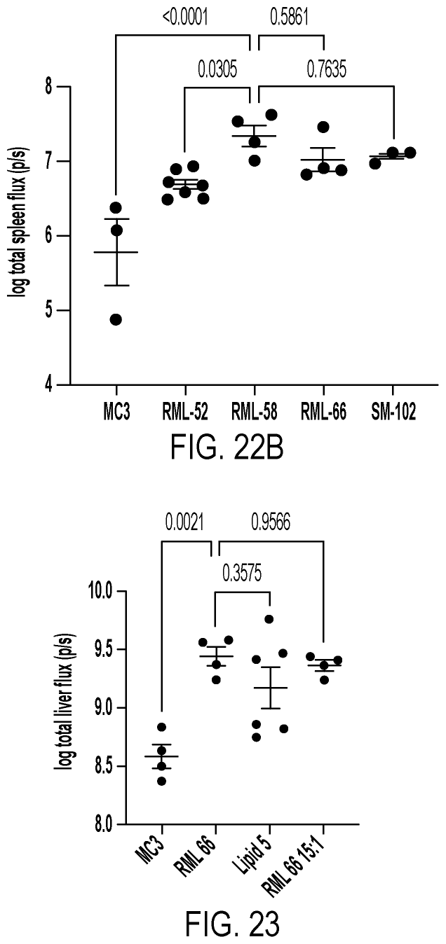

RML52, RML58, and RML66 were tested against the benchmark lipids DLin-MC3-DMA (“MC3”) and against SM-102, Moderna's FDA approved lipid for IM mRNA delivery.²² Each of these lipids was significantly better than MC3, and RML66 performed similarly to SM-102 (FIG. 22A). Interestingly, robust spleen delivery was observed for RML58 (FIG. 22B).

Next, RML66 was tested at a higher dose (0.8 mg/kg). It performed substantially better than MC3 and similarly to Lipid 5, a liver-optimized SM-102 analog (FIG. 23).²³ A reduced ionizable lipid:mRNA weight ratio of 15:1 exhibited either no differences or small differences from the 20:1 ratio (FIG. 23).

### REFERENCES

(1) Akinc, A.; Zumbuehl, A.; Goldberg, M.; Leshchiner, E. S.; Busini, V.; Hossain, N.; Bacallado, S. A.; Nguyen, D. N.; Fuller, J.; Alvarez, R.; Borodovsky, A.; Borland, T.; Constien, R.; de Fougerolles, A.; Dorkin, J. R.; Narayanannair Jayaprakash, K.; Jayaraman, M.; John, M.; Koteliansky, V.; Manoharan, M.; Nechev, L.; Qin, J.; Racie, T.; Raitcheva, D.; Rajeev, K. G.; Sah, D. W. Y.; Soutschek, J.; Toudjarska, I.; Vornlocher, H.-P.; Zimmermann, T. S.; Langer, R.; Anderson, D. G. A Combinatorial Library of Lipid-like Materials for Delivery of RNAi Therapeutics. Nat. Biotechnol. 2008, 26, 561.
(2) Whitehead, K. A.; Dorkin, J. R.; Vegas, A. J.; Chang, P. H.; Veiseh, O.; Matthews, J.; Fenton, O. S.; Zhang, Y.; Olejnik, K. T.; Yesilyurt, V.; Chen, D.; Barros, S.; Klebanov, B.; Novobrantseva, T.; Langer, R.; Anderson, D. G. Degradable Lipid Nanoparticles with Predictable in Vivo SiRNA Delivery Activity. Nat. Commun. 2014, 5, 4277.
(3) Liu, J.; Chang, J.; Jiang, Y.; Meng, X.; Sun, T.; Mao, L.; Xu, Q.; Wang, M. Fast and Efficient CRISPR/Cas9 Genome Editing In Vivo Enabled by Bioreducible Lipid and Messenger RNA Nanoparticles. Adv. Mater. 2019, 31 (33), 1902575. https://doi.org/https://doi.org/10.1002/adma.201902575.
(4) Maier, M. A.; Jayaraman, M.; Matsuda, S.; Liu, J.; Barros, S.; Querbes, W.; Tam, Y. K.; Ansell, S. M.; Kumar, V.; Qin, J.; Zhang, X.; Wang, Q.; Panesar, S.; Hutabarat, R.; Carioto, M.; Hettinger, J.; Kandasamy, P.; Butler, D.; Rajeev, K. G.; Pang, B.; Charisse, K.; Fitzgerald, K.; Mui, B. L.; Du, X.; Cullis, P.; Madden, T. D.; Hope, M. J.; Manoharan, M.; Akinc, A. Biodegradable Lipids Enabling Rapidly Eliminated Lipid Nanoparticles for Systemic Delivery of RNAi Therapeutics. Mol. Ther. 2013, 21 (8), 1570-1578. https://doi.org/10.1038/mt.2013.124.
(5) Sabnis, S.; Kumarasinghe, E. S.; Salerno, T.; Mihai, C.; Ketova, T.; Senn, J. J.; Lynn, A.; Bulychev, A.; McFadyen, I.; Chan, J.; Almarsson, Ö.; Stanton, M. G.; Benenato, K. E. A Novel Amino Lipid Series for MRNA Delivery: Improved Endosomal Escape and Sustained Pharmacology and Safety in Non-Human Primates. Mol. Ther. 2018, 26 (6), 1509-1519. https://doi.org/10.1016/j.ymthe.2018.03.010.
(6) Jayaraman, M.; Ansell, S. M.; Mui, B. L.; Tam, Y. K.; Chen, J.; Du, X.; Butler, D.; Eltepu, L.; Matsuda, S.; Narayanannair, J. K.; Rajeev, K. G.; Hafez, I. M.; Akinc, A.; Maier, M. A.; Tracy, M. A.; Cullis, P. R.; Madden, T. D.; Manoharan, M.; Hope, M. J. Maximizing the Potency of SiRNA Lipid Nanoparticles for Hepatic Gene Silencing In Vivo. Angew. Chemie Int. Ed. 2012, 51 (34), 8529-8533. https://doi.org/https://doi.org/10.1002/anie.201203263.
(7) Ghanem, R.; Laurent, V.; Roquefort, P.; Haute, T.; Ramel, S.; Le Gall, T.; Aubry, T.; Montier, T. Optimizations of In Vitro Mucus and Cell Culture Models to Better Predict In Vivo Gene Transfer in Pathological Lung Respiratory Airways: Cystic Fibrosis as an Example. Pharmaceutics. 2021. https://doi.org/10.3390/pharmaceutics13010047.
(8) Yin, H.; Song, C.-Q.; Suresh, S.; Wu, Q.; Walsh, S.; Rhym, L. H.; Mintzer, E.; Bolukbasi, M. F.; Zhu, L. J.; Kauffman, K.; Mou, H.; Oberholzer, A.; Ding, J.; Kwan, S.-Y.; Bogorad, R. L.; Zatsepin, T.; Koteliansky, V.; Wolfe, S. A.; Xue, W.; Langer, R.; Anderson, D. G. Structure-Guided Chemical Modification of Guide RNA Enables Potent Non-Viral in Vivo Genome Editing. Nat. Biotechnol. 2017, 35 (12), 1179-1187. https://doi.org/10.1038/nbt.4005.
(9) Patel, A. K.; Kaczmarek, J. C.; Bose, S.; Kauffman, K. J.; Mir, F.; Heartlein, M. W.; DeRosa, F.; Langer, R.; Anderson, D. G. Inhaled Nanoformulated MRNA Polyplexes for Protein Production in Lung Epithelium. Adv. Mater. 2019, 31 (8), 1805116. https://doi.org/10.1002/adma.201805116.
(10) Finn, J. D.; Smith, A. R.; Patel, M. C.; Shaw, L.; Youniss, M. R.; van Heteren, J.; Dirstine, T.; Ciullo, C.; Lescarbeau, R.; Seitzer, J.; Shah, R. R.; Shah, A.; Ling, D.; Growe, J.; Pink, M.; Rohde, E.; Wood, K. M.; Salomon, W. E.; Harrington, W. F.; Dombrowski, C.; Strapps, W. R.; Chang, Y.; Morrissey, D. V. A Single Administration of CRISPR/Cas9 Lipid Nanoparticles Achieves Robust and Persistent Genome Editing. Cell Rep. 2018, 22 (9), 2227-2235. https://doi.org/10.1016/i.celrep.2018.02.014.
(11) Zhao, P.; Hou, X.; Yan, J.; Du, S.; Xue, Y.; Li, W.; Xiang, G.; Dong, Y. Long-Term Storage of Lipid-like Nanoparticles for MRNA Delivery. Bioact. Mater. 2020, 5 (2), 358-363. https://doi.org/https://doi.org/10.1016/j.bioactmat.2020.03.001.
(12) Shetty, N.; Cipolla, D.; Park, H.; Zhou, Q. T. Physical Stability of Dry Powder Inhaler Formulations. Expert Opin. Drug Deliv. 2020, 17 (1), 77-96. https://doi.org/10.1080/17425247.2020.1702643.
(13) Sasaki, S. & Guo, S. Nucleic acid therapies for cystic fibrosis. Nucleic Acid Therapeutics vol. 28 1-9 (2018).
(14) Watts, J. K., Brown, R. H. & Khvorova, A. Nucleic Acid Therapeutics for Neurological Diseases. Neurotherapeutics vol. 16 245-247 (2019).
(15) Mukalel, A. J., Riley, R. S., Zhang, R. & Mitchell, M. J. Nanoparticles for nucleic acid delivery: Applications in cancer immunotherapy. Cancer Letters vol. 458 102-112 (2019).
(16) Study to Evaluate the Safety & Tolerability of MRT5005 Administered by Nebulization in Adults With Cystic Fibrosis—Full Text View—ClinicalTrials.gov. https://clinicaltrials.gov/ct2/show/NCT03375047.
(17) Rural hospitals can't afford freezers to store a Covid-19 vaccine—STAT. https://www.statnews.com/2020/11/11/rural-hospitals-cant-afford-freezers-to-store-pfizer-covid19-vaccine/.
(18) Pfizer COVID vaccine needs super-cold storage. But that could change|Fortune. https://fortune.com/2020/11/28/pfizer-covid-vaccine-cold-storage-update/.
(19) How Long It Will Take to Get Covid-19 Vaccines to Most Americans—WSJ. https://www.wsj.com/articles/where-our-current-covid-19-vaccination-rate-will-take-us-11611324000.
(20) Ball, R., Bajaj, P. & Whitehead, K. Achieving long-term stability of lipid nanoparticles: examining the effect of pH, temperature, and lyophilization. Int. J. Nanomedicine Volume 12, 305-315 (2016).
(21) Vanbever, R. et al. Formulation and physical characterization of large porous particles for inhalation. Pharm. Res. 16, 1735-1742 (1999).
(22) Han, X. et al. An ionizable lipid toolbox for RNA delivery. Nat. Commun. 12, 7233 (2021).
(23) Sabnis, S. et al. A Novel Amino Lipid Series for mRNA Delivery: Improved Endosomal Escape and Sustained Pharmacology and Safety in Non-human Primates. Mol. Ther. 26, 1509-1519 (2018).

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 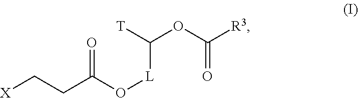
or a pharmaceutically acceptable salt, solvate, tautomer, stereoisomer, or isotopically labeled derivative thereof, wherein:
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl;
T is optionally substituted C₁-C₂₀ alkyl, optionally substituted C₂-C₂₀ alkenyl, or optionally substituted C₂-C₂₀ alkynyl;
L is optionally substituted C₁-C₂₀ alkylene, optionally substituted C₂-C₂₀ alkenylene, or optionally substituted C₂-C₂₀ alkynylene; and
R³ is optionally substituted aliphatic or optionally substituted heteroaliphatic.

2. The compound of claim 1, wherein the compound of Formula (I) is of Formula (II): 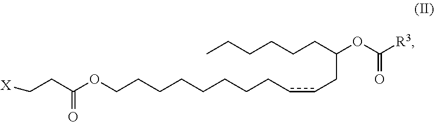
or a pharmaceutically acceptable salt, solvate, tautomer, stereoisomer, or isotopically labeled derivative thereof, wherein:
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl;
R³ is optionally substituted aliphatic or optionally substituted heteroaliphatic; and 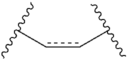
represents either a single or a double bond.

3. The compound of claim 2, or a pharmaceutically acceptable salt, solvate, tautomer, stereoisomer, or isotopically labeled derivative thereof, of Formula (II-A):

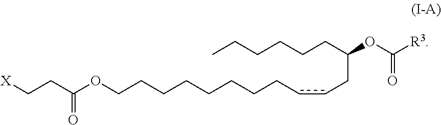

4. The compound of claim 2, or a pharmaceutically acceptable salt, solvate, tautomer, stereoisomer, or isotopically labeled derivative thereof, of Formula (II-B):

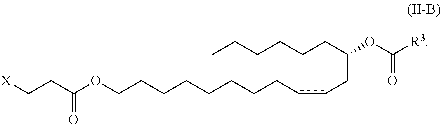

5. The compound of any one of claims 2-4, or a pharmaceutically acceptable salt, solvate, tautomer, stereoisomer, or isotopically labeled derivative thereof, of Formula (II-C):

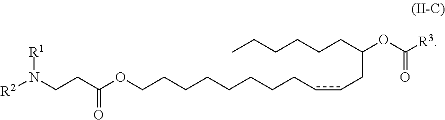

6. The compound of any one of claims 2-5, wherein R¹ is —H, optionally substituted alkyl, optionally substituted heteroalkyl, or optionally substituted heterocyclyl.

7. The compound of claim 6, wherein R¹ is -Me or -Et.

8. The compound of any one of claims 2-5, or a pharmaceutically acceptable salt, solvate, tautomer, stereoisomer, or isotopically labeled derivative thereof, wherein R¹ and R² are joined together with the intervening atoms to form an optionally substituted heterocycle.

9. The compound of claim 8, or a pharmaceutically acceptable salt, solvate, tautomer, stereoisomer, or isotopically labeled derivative thereof, wherein X is selected from the group consisting of

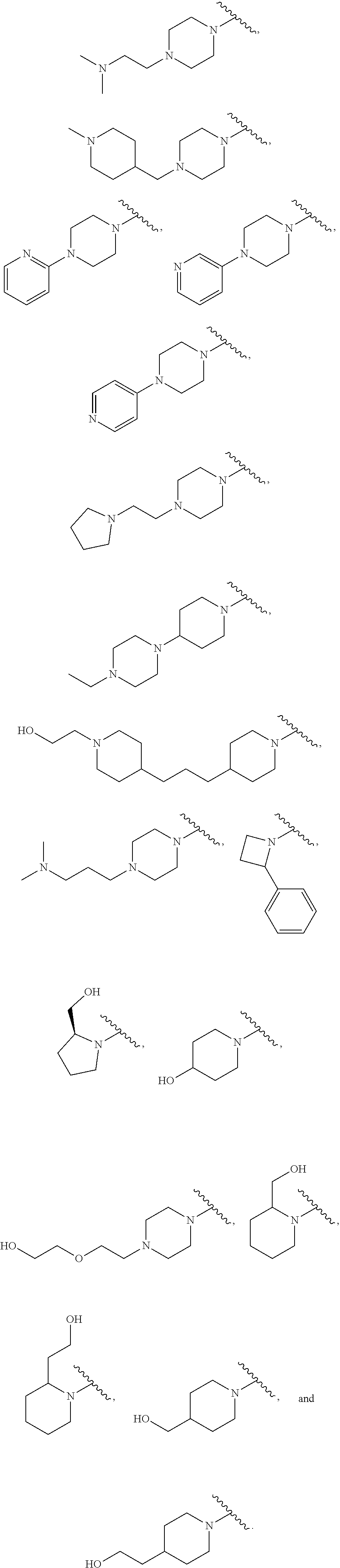

10. The compound of any one of claims 2-5, or a pharmaceutically acceptable salt, solvate, tautomer, stereoisomer, or isotopically labeled derivative thereof, of Formula (II-D):

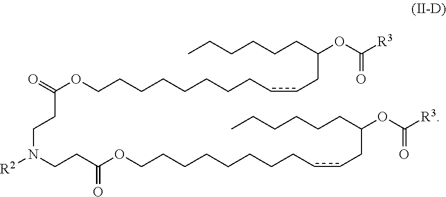

11. The compound of any one of claims 2-7 or 10, wherein R² is —H, optionally substituted alkyl, optionally substituted heteroalkyl, or optionally substituted heterocyclyl.

12. The compound of claim 11, or a pharmaceutically acceptable salt, solvate, tautomer, stereoisomer, or isotopically labeled derivative thereof, wherein R² is selected from the group consisting of -Me,

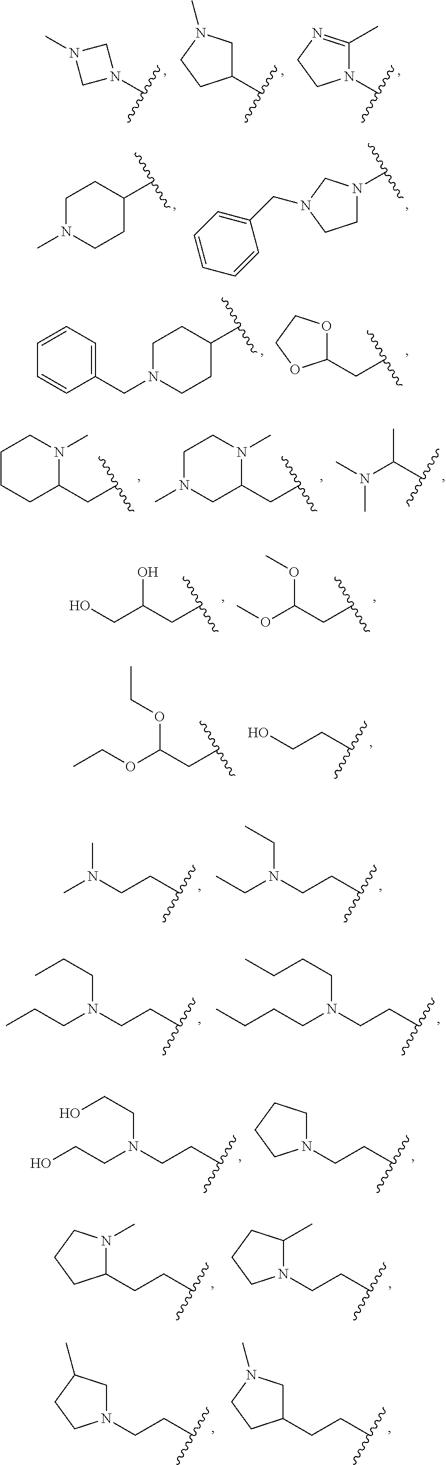

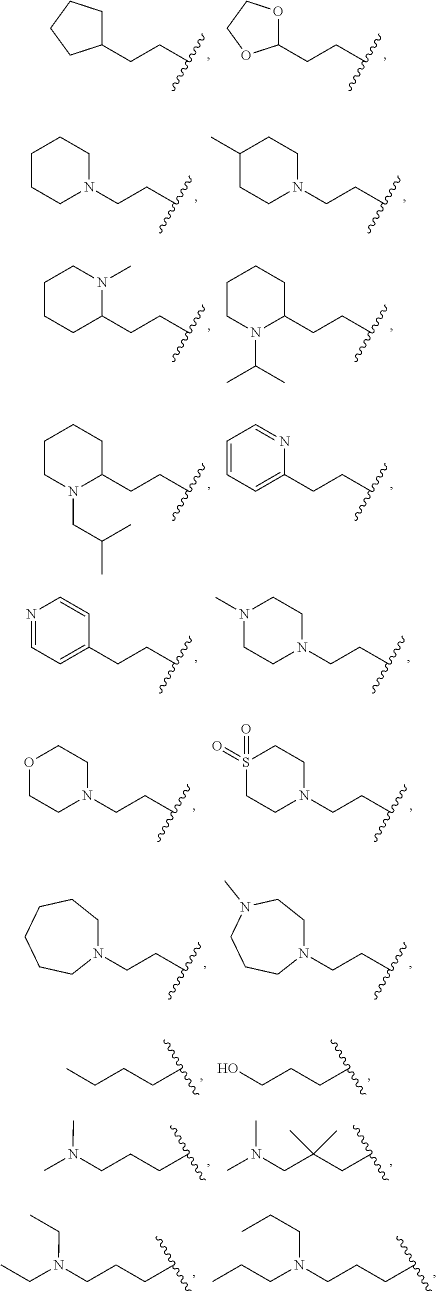

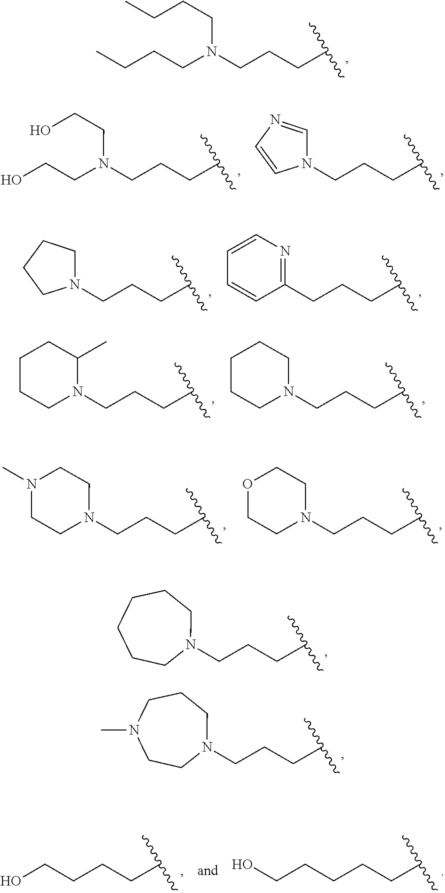

13. The compound of claim 5, or a pharmaceutically acceptable salt, solvate, hydrate, polymorph, co-crystal, tautomer, wherein R¹ and R² are joined together with the intervening atoms to form an optionally substituted heterocycle comprising one or more N atoms substituted with

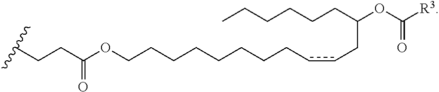

14. The compound of claim 13, or a pharmaceutically acceptable salt, solvate, hydrate, polymorph, co-crystal, tautomer, wherein R¹ and R² are joined together with the intervening atoms to form an optionally substituted heterocycle comprising two N atoms substituted with

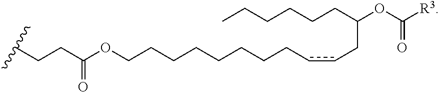

15. The compound of claim 13, or a pharmaceutically acceptable salt, solvate, hydrate, polymorph, co-crystal, tautomer, wherein R¹ and R² are joined together with the intervening atoms to form an optionally substituted heterocycle comprising three N atoms substituted with

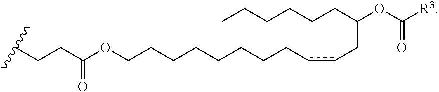

16. The compound of claim 5, or a pharmaceutically acceptable salt, solvate, tautomer, stereoisomer, or isotopically labeled derivative thereof, wherein R² is optionally substituted heteroalkyl comprising one or more N atoms substituted with

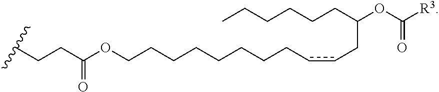

17. The compound of any one of claims 2-16, or a pharmaceutically acceptable salt, solvate, tautomer, stereoisomer, or isotopically labeled derivative thereof, wherein 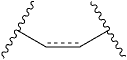
represents a single bond.

18. The compound of any one of claims 2-16, or a pharmaceutically acceptable salt, solvate, tautomer, stereoisomer, or isotopically labeled derivative thereof, wherein 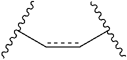
represents a double bond.

19. The compound of any one of the preceding claims, or a pharmaceutically acceptable salt, solvate, tautomer, stereoisomer, or isotopically labeled derivative thereof, wherein R³ is C₁-C₂₅ optionally substituted aliphatic.

20. The compound of claim 19, or a pharmaceutically acceptable salt, solvate, tautomer, stereoisomer, or isotopically labeled derivative thereof, wherein R³ is selected from the group consisting of

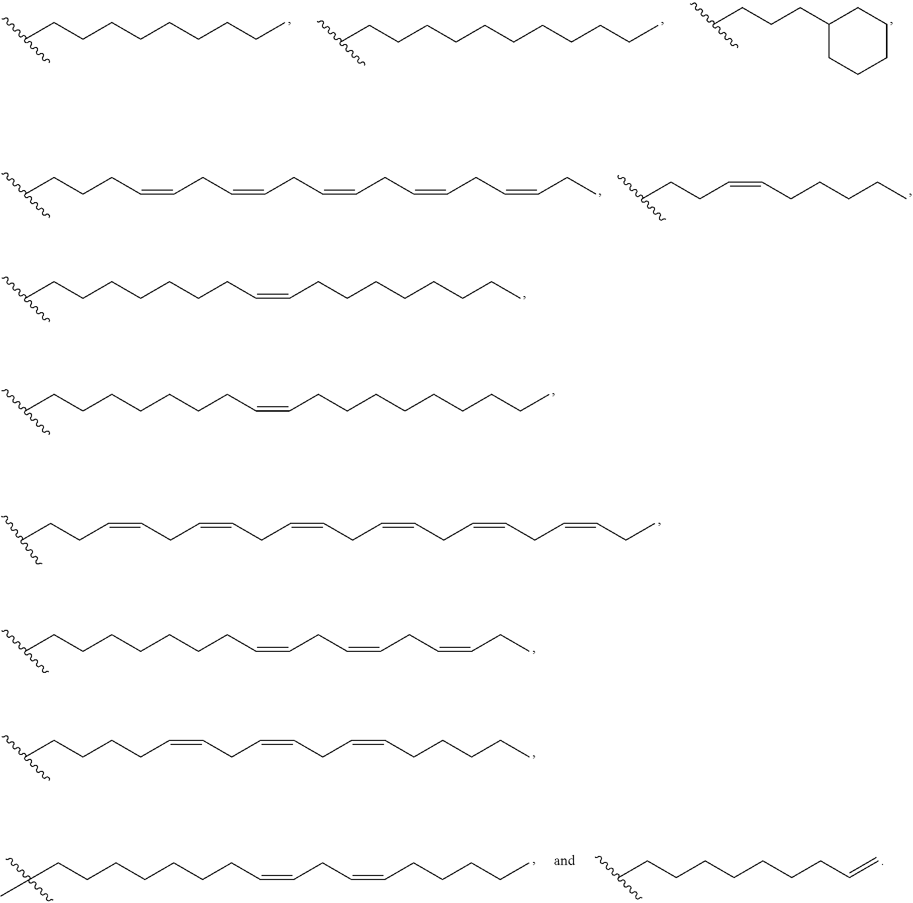

21. The compound of any one of claims 1-18, or a pharmaceutically acceptable salt, solvate, tautomer, stereoisomer, or isotopically labeled derivative thereof, wherein R³ is optionally substituted C₁-C₂₅ heteroaliphatic.

22. The compound of claim 21, or a pharmaceutically acceptable salt, solvate, tautomer, stereoisomer, or isotopically labeled derivative thereof, wherein R³ is —OR^{3O}, wherein R^{3O} is optionally substituted C₁-C₂₅ aliphatic.

23. The compound of claim 22, or a pharmaceutically acceptable salt, solvate, tautomer, stereoisomer, or isotopically labeled derivative thereof, wherein R^{3O} is selected from the group consisting of

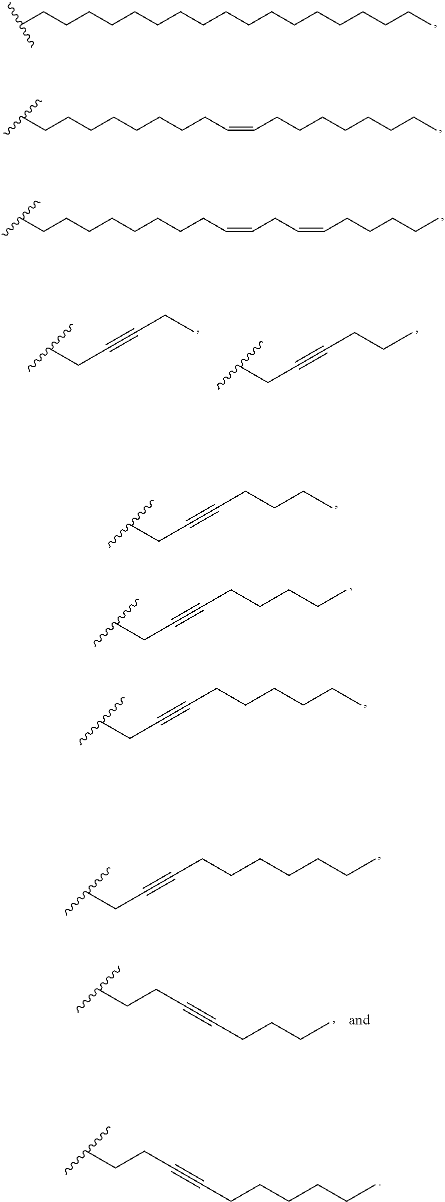

24. The compound of claim 21, or a pharmaceutically acceptable salt, solvate, tautomer, stereoisomer, or isotopically labeled derivative thereof, wherein R³ is —N(R^{3N})₂, wherein each instance of R^{3N} is independently optionally substituted C₁-C₂₅ aliphatic.

25. The compound of claim 2, selected from 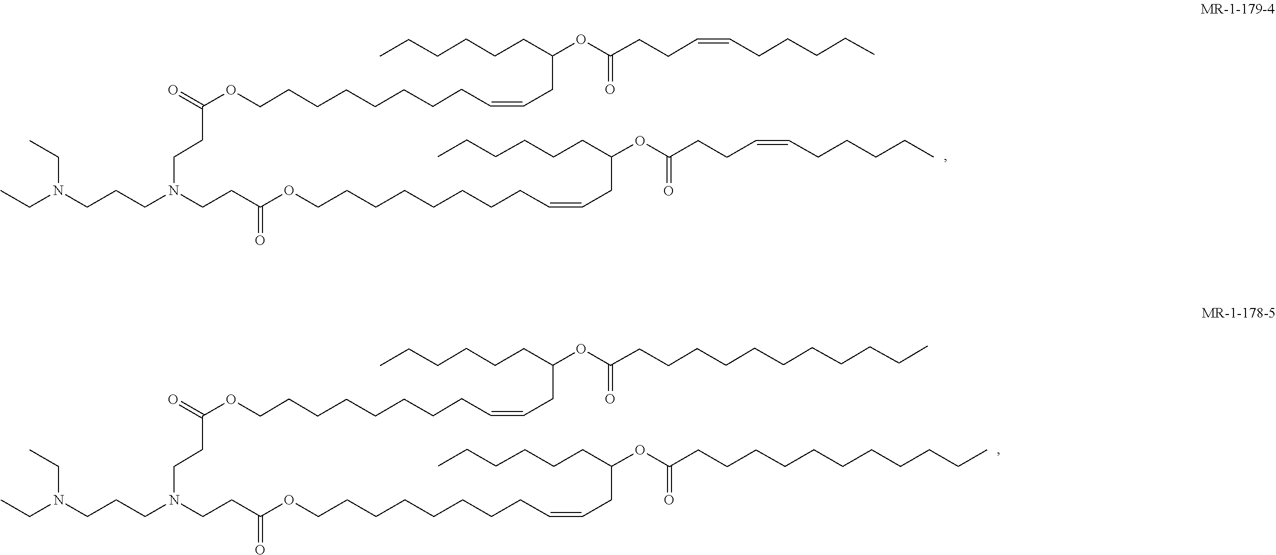 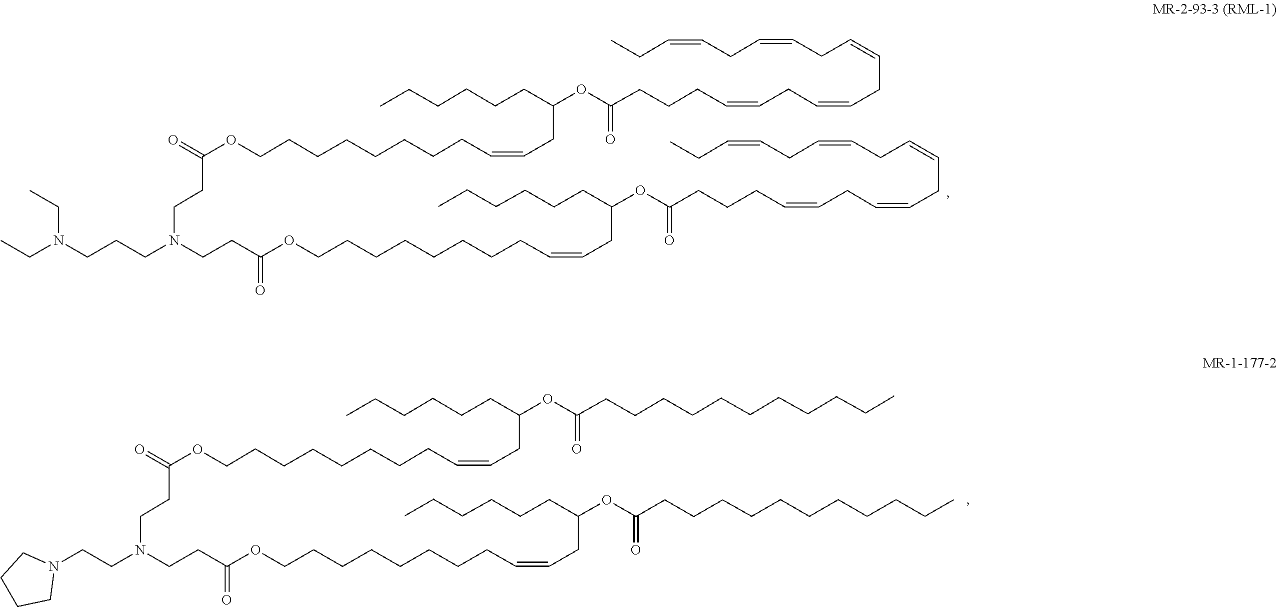 [SMILES: CC/C=C\C/C=C\C/C=C\C/C=C\C/C=C\CCCC(=O)OC(C/C=C\CCCCCCCCOC(=O)CCN(CCCN(CC)CC)CCC(=O)OCCCCCCCC/C=C\CC(CCCCCC)OC(=O)CCC/C=C\C/C=C\C/C=C\C/C=C\C/C=C\CC)CCCCCC ; CCCCCCCCCCCC(=O)OC(C/C=C\CCCCCCCCOC(=O)CCN(CCC(=O)OCCCCCCCC/C=C\CC(CCCCCC)OC(=O)CCCCCCCCCCC)CCN1CCCC1)CCCCCC] 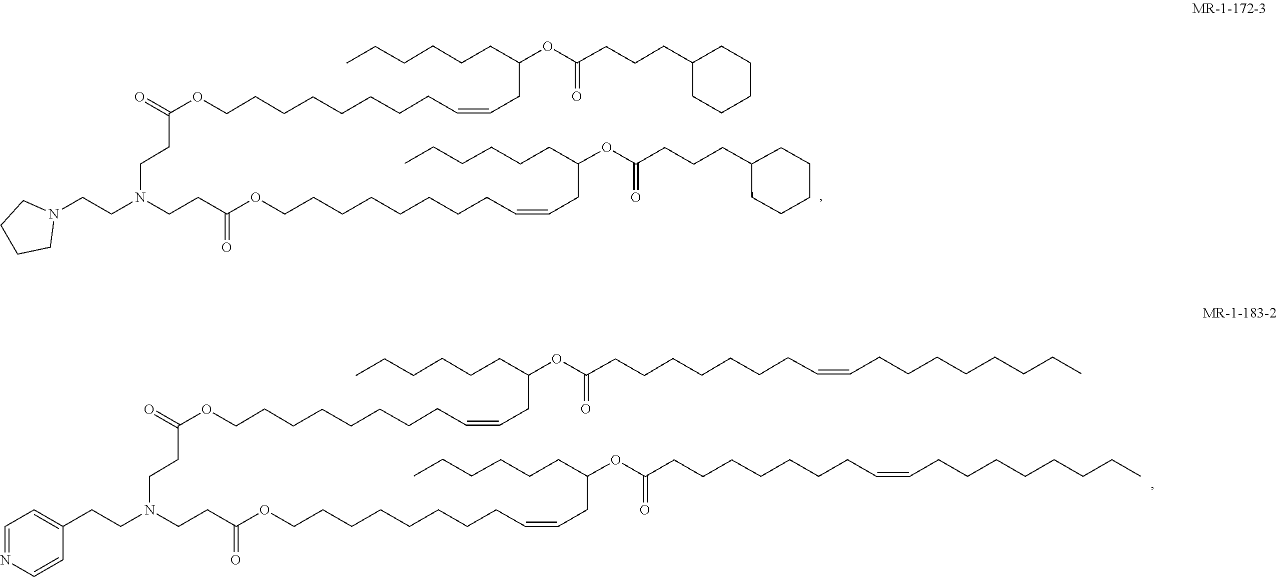 [SMILES: CCCCCCC(C/C=C\CCCCCCCCOC(=O)CCN(CCC(=O)OCCCCCCCC/C=C\CC(CCCCCC)OC(=O)CCCC1CCCCC1)CCN1CCCC1)OC(=O)CCCC1CCCCC1 ; CCCCCCCC/C=C\CCCCCCCC(=O)OC(C/C=C\CCCCCCCCOC(=O)CCN(CCC(=O)OCCCCCCCC/C=C\CC(CCCCCC)OC(=O)CCCCCCC/C=C\CCCCCCCC)CCc1ccncc1)CCCCCC] 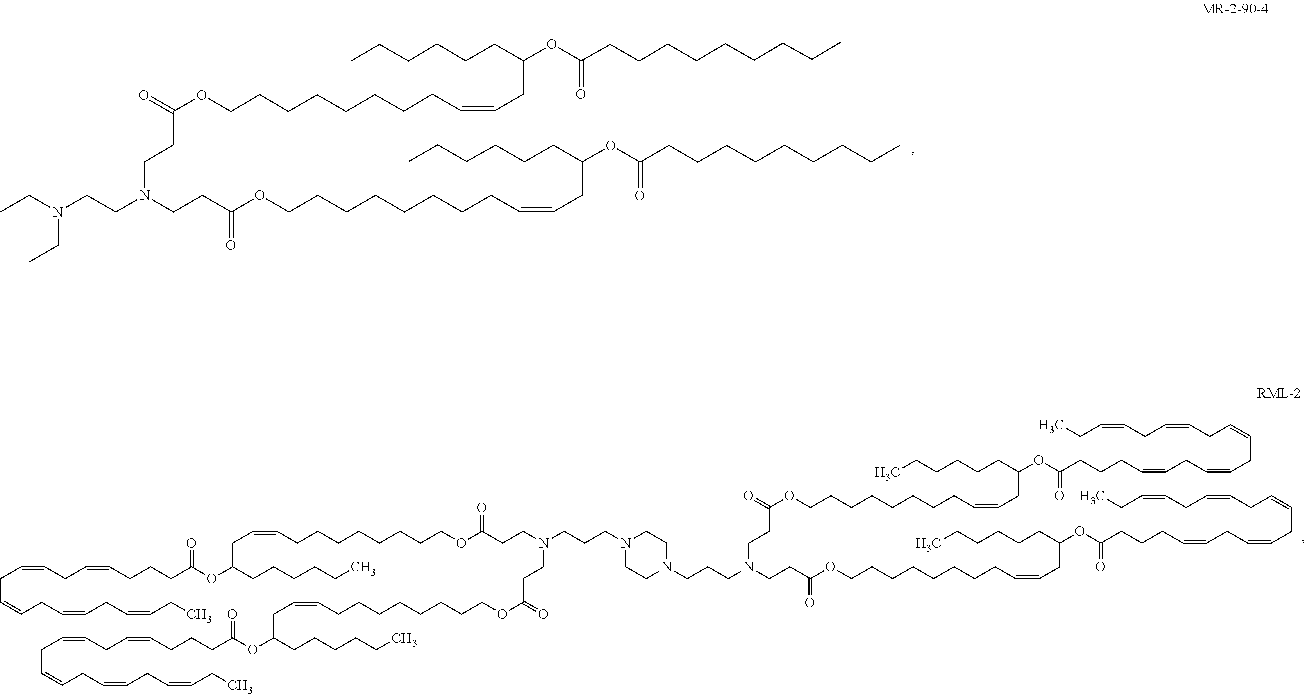 [SMILES: CCCCCCCCCC(=O)OC(C/C=C\CCCCCCCCOC(=O)CCN(CCC(=O)OCCCCCCCC/C=C\CC(CCCCCC)OC(=O)CCCCCCCCC)CCN(CC)CC)CCCCCC ; CC/C=C\C/C=C\C/C=C\C/C=C\C/C=C\CCCC(=O)OC(C/C=C\CCCCCCCCOC(=O)CCN(CCCN1CCN(CCCN(CCC(=O)OCCCCCCCC/C=C\CC(CCCCCC)OC(=O)CCC/C=C\C/C=C\C/C=C\C/C=C\C/C=C\CC)CCC(=O)OCCCCCCCC/C=C\CC(CCCCCC)OC(=O)CCC/C=C\C/C=C\C/C=C\C/C=C\C/C=C\CC)CC1)CCC(=O)OCCCCCCCC/C=C\CC(CCCCCC)OC(=O)CCC/C=C\C/C=C\C/C=C\C/C=C\C/C=C\CC)CCCCCC] 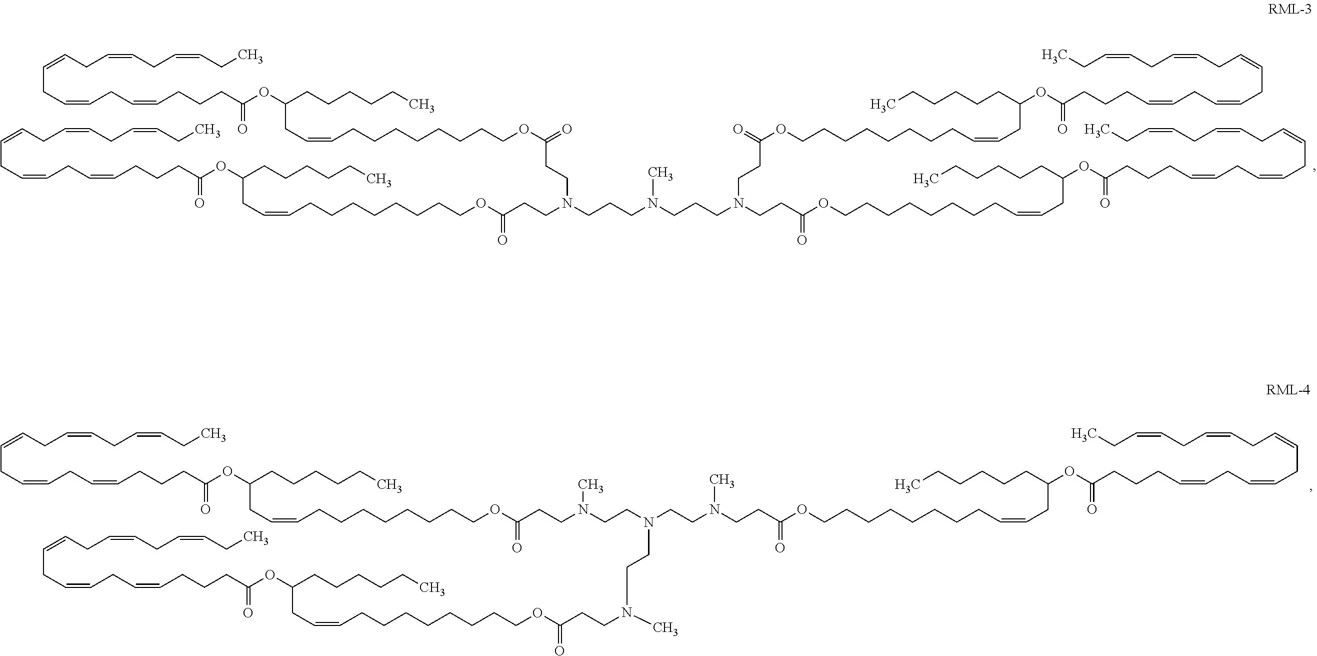 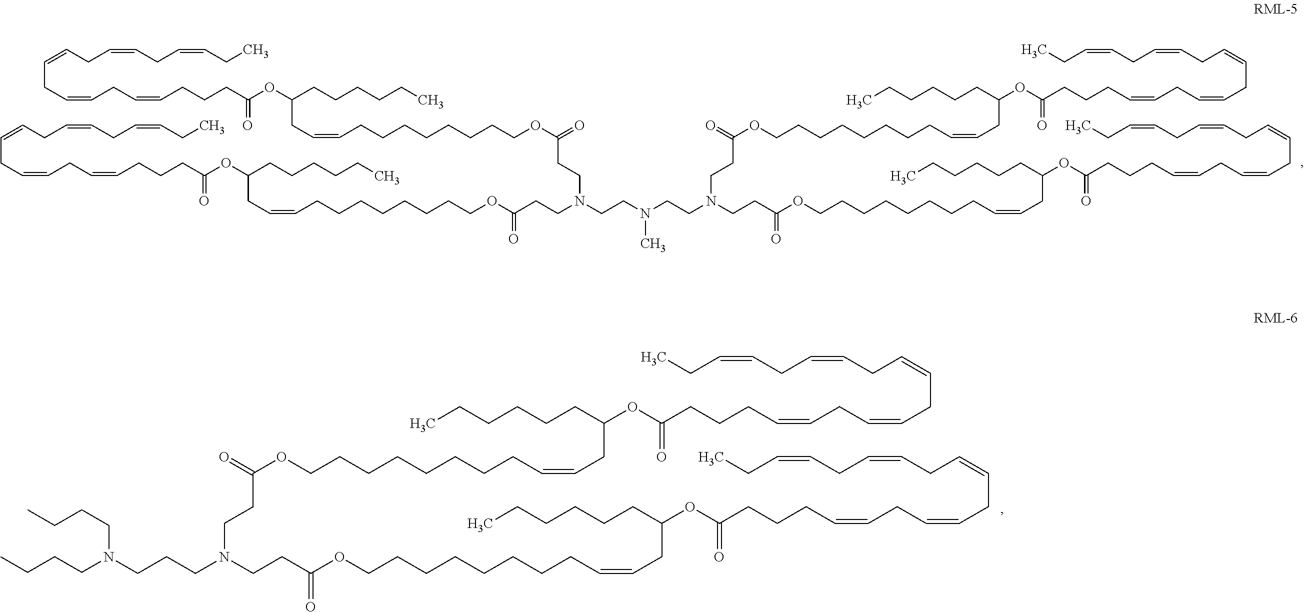 [SMILES: CC/C=C\C/C=C\C/C=C\C/C=C\C/C=C\CCCC(=O)OC(C/C=C\CCCCCCCCOC(=O)CCN(CCC(=O)OCCCCCCCC/C=C\CC(CCCCCC)OC(=O)CCC/C=C\C/C=C\C/C=C\C/C=C\C/C=C\CC)CCN(C)CCN(CCC(=O)OCCCCCCCC/C=C\CC(CCCCCC)OC(=O)CCC/C=C\C/C=C\C/C=C\C/C=C\C/C=C\CC)CCC(=O)OCCCCCCCC/C=C\CC(CCCCCC)OC(=O)CCC/C=C\C/C=C\C/C=C\C/C=C\C/C=C\CC)CCCCCC ; CC/C=C\C/C=C\C/C=C\C/C=C\C/C=C\CCCC(=O)OC(C/C=C\CCCCCCCCOC(=O)CCN(CCCN(CCCC)CCCC)CCC(=O)OCCCCCCCC/C=C\CC(CCCCCC)OC(=O)CCC/C=C\C/C=C\C/C=C\C/C=C\C/C=C\CC)CCCCCC] 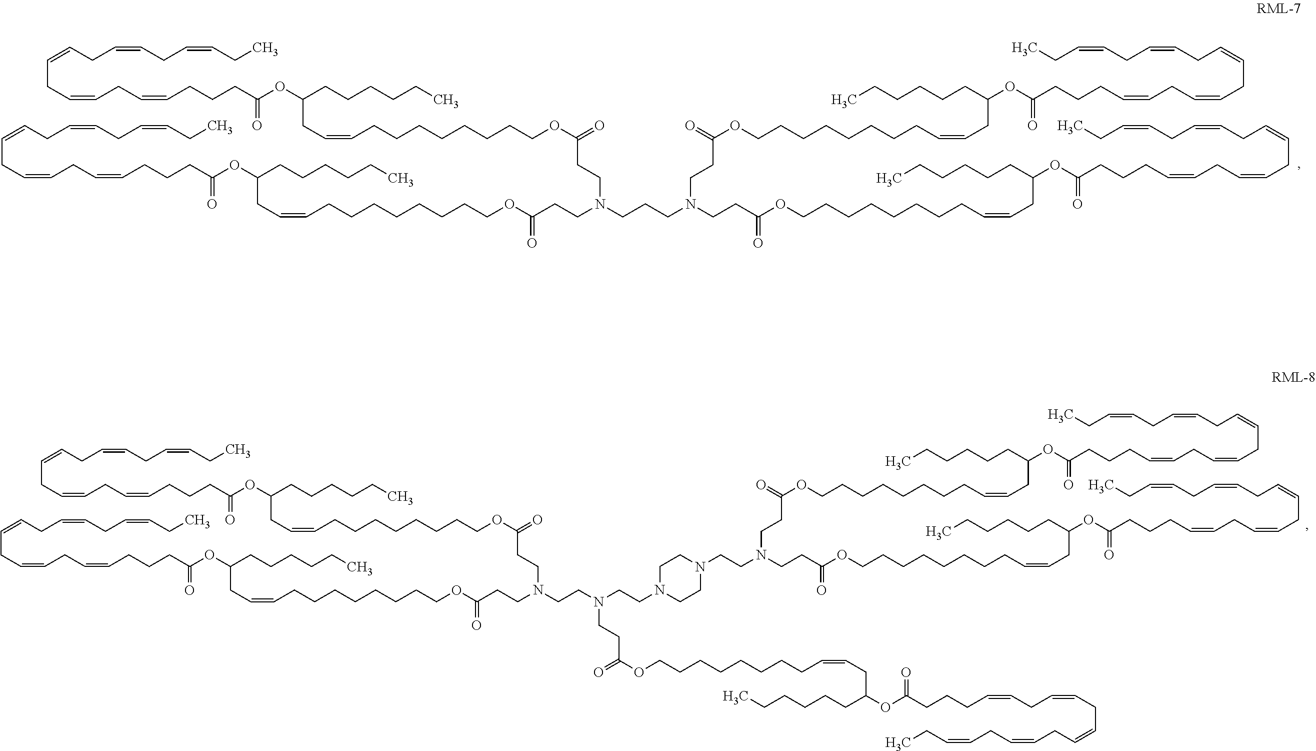 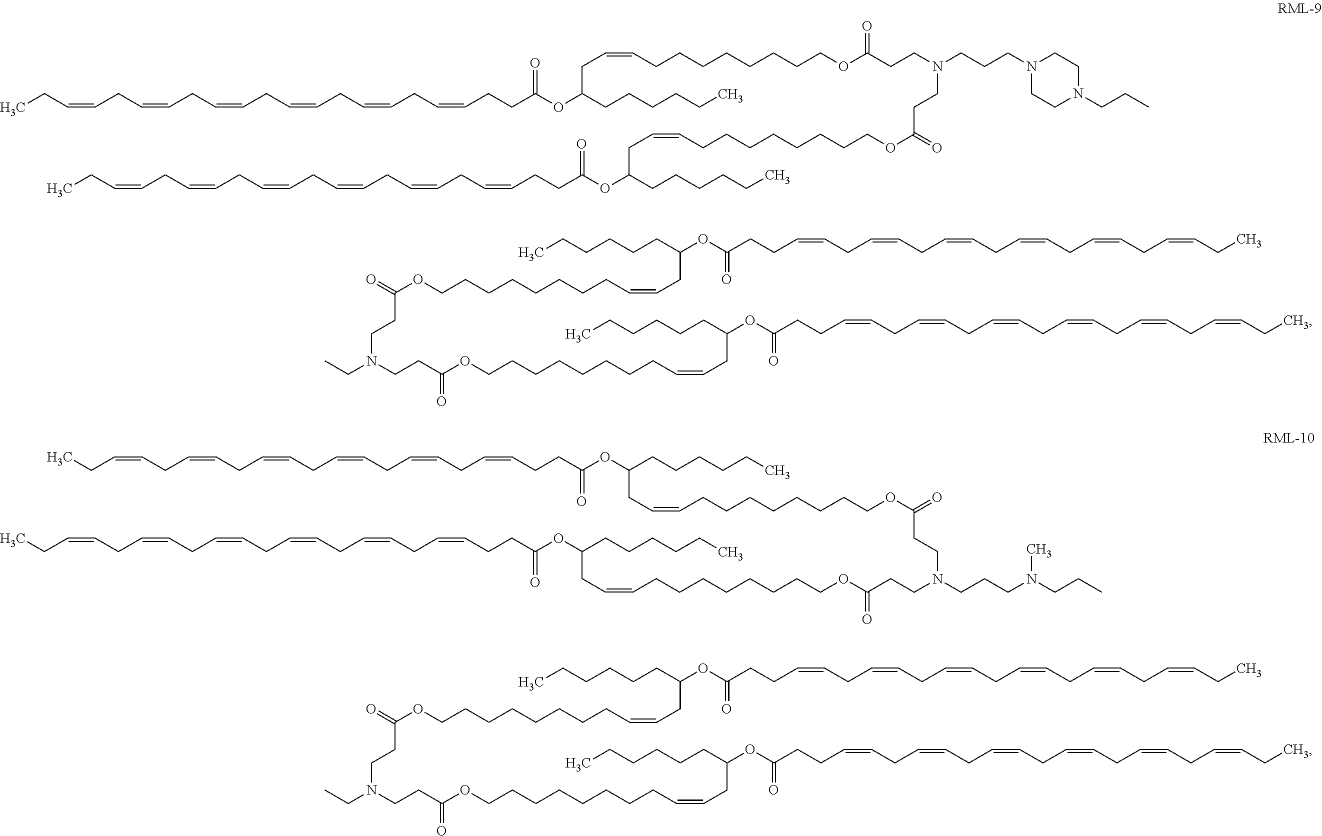 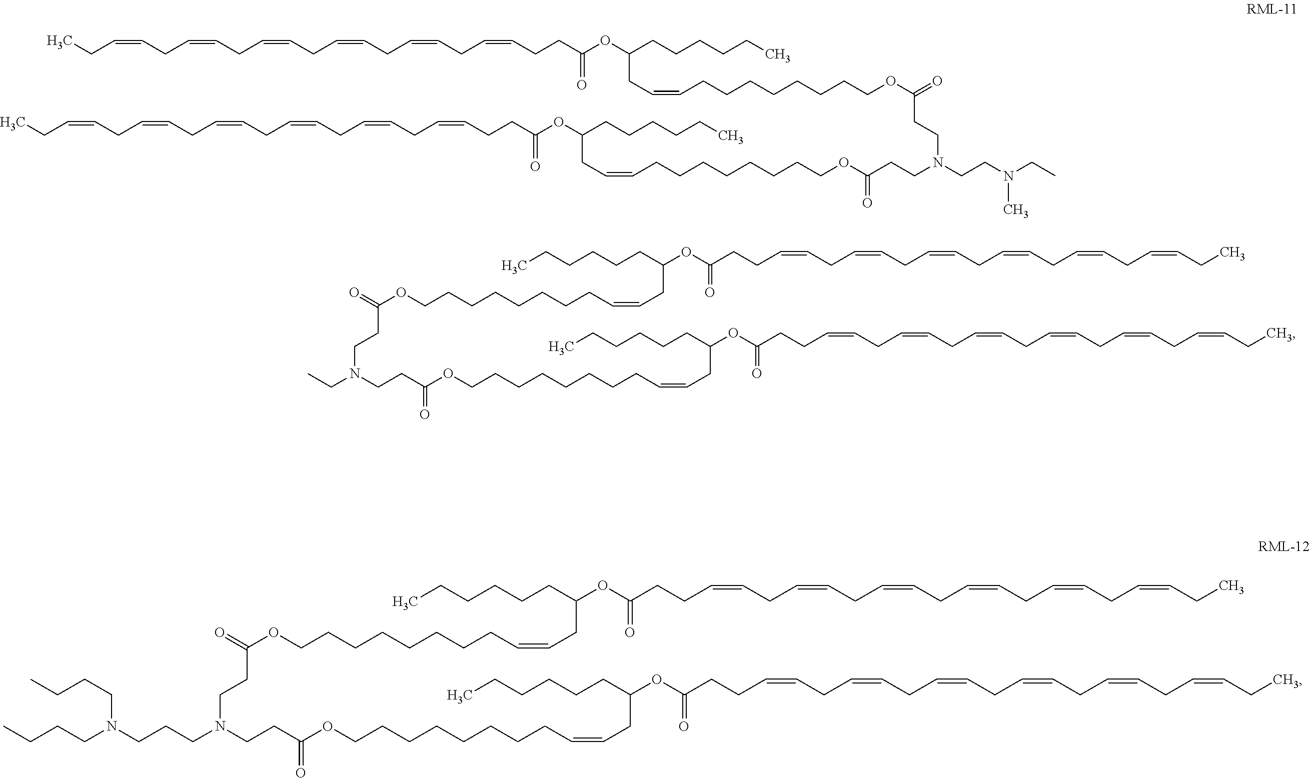 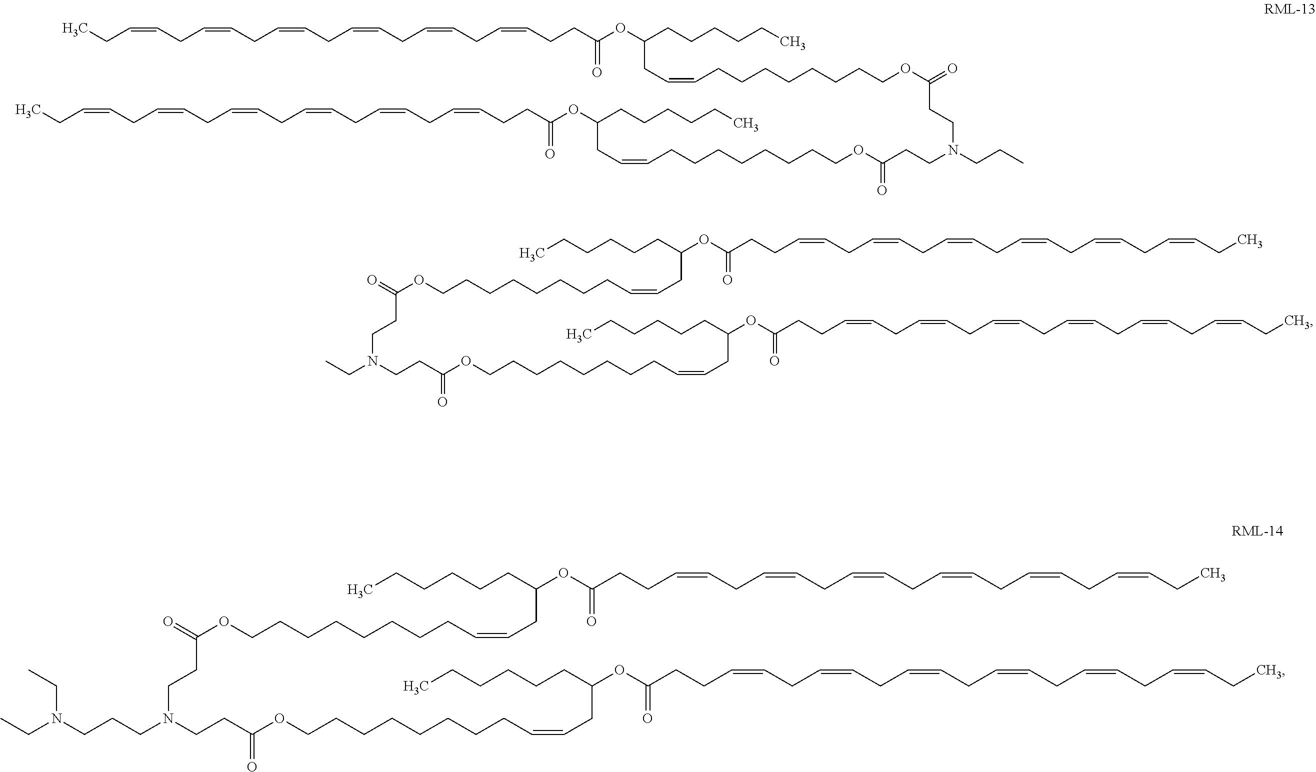 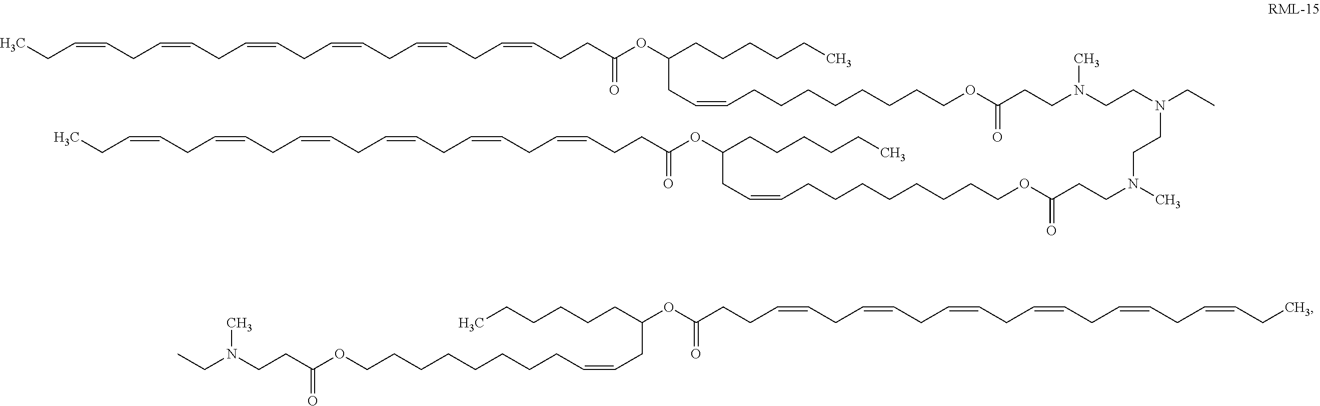 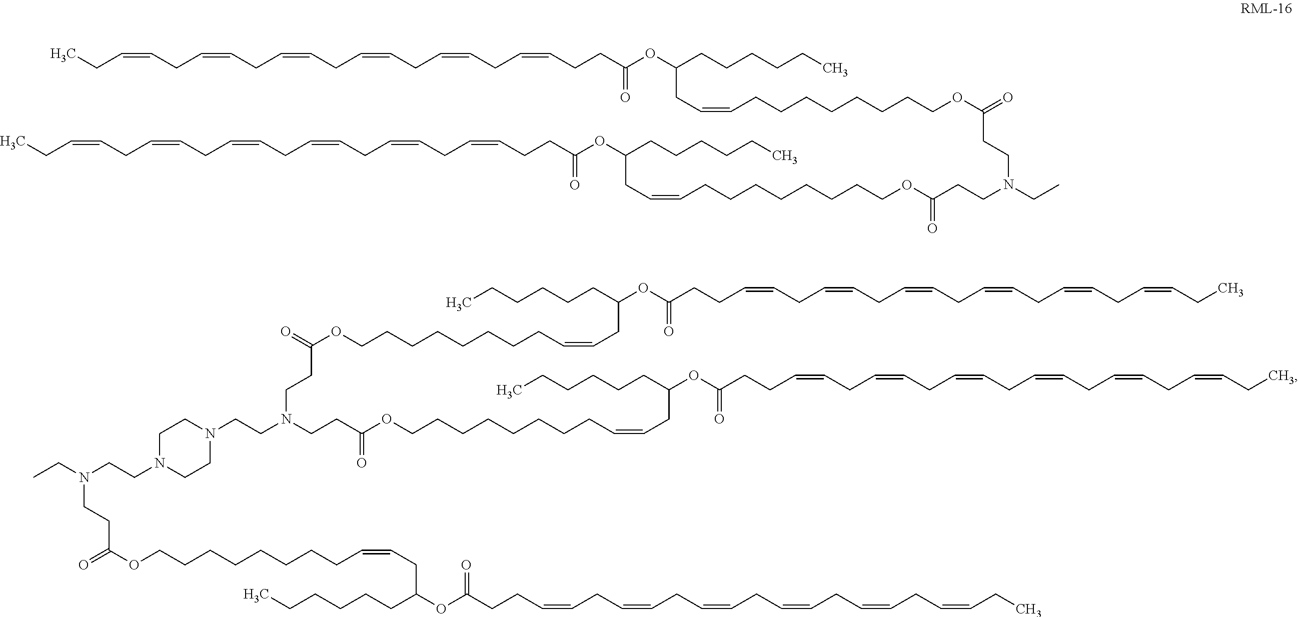 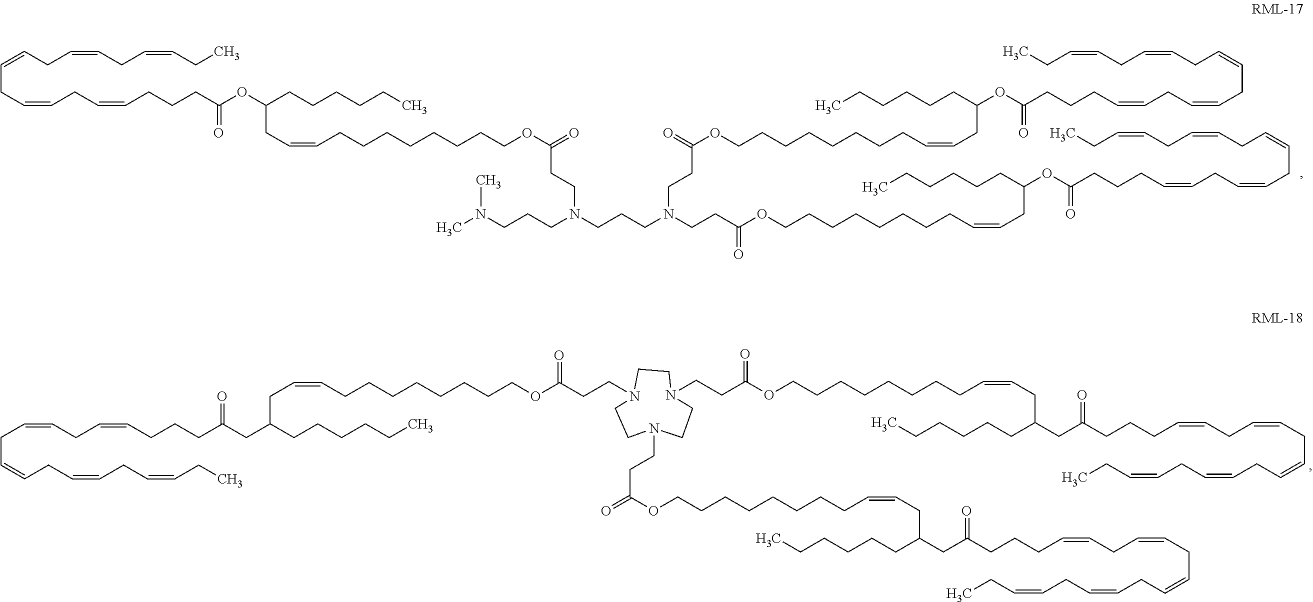 [SMILES: CC/C=C\C/C=C\C/C=C\C/C=C\C/C=C\CCCC(=O)OC(C/C=C\CCCCCCCCOC(=O)CCN(CCCN(C)C)CCCN(CCC(=O)OCCCCCCCC/C=C\CC(CCCCCC)OC(=O)CCC/C=C\C/C=C\C/C=C\C/C=C\C/C=C\CC)CCC(=O)OCCCCCCCC/C=C\CC(CCCCCC)OC(=O)CCC/C=C\C/C=C\C/C=C\C/C=C\C/C=C\CC)CCCCCC ; CC/C=C\C/C=C\C/C=C\C/C=C\C/C=C\CCCC(=O)CC(C/C=C\CCCCCCCCOC(=O)CCN1CCN(CCC(=O)OCCCCCCCC/C=C\CC(CCCCCC)CC(=O)CCC/C=C\C/C=C\C/C=C\C/C=C\C/C=C\CC)CCN(CCC(=O)OCCCCCCCC/C=C\CC(CCCCCC)CC(=O)CCC/C=C\C/C=C\C/C=C\C/C=C\C/C=C\CC)CC1)CCCCCC] 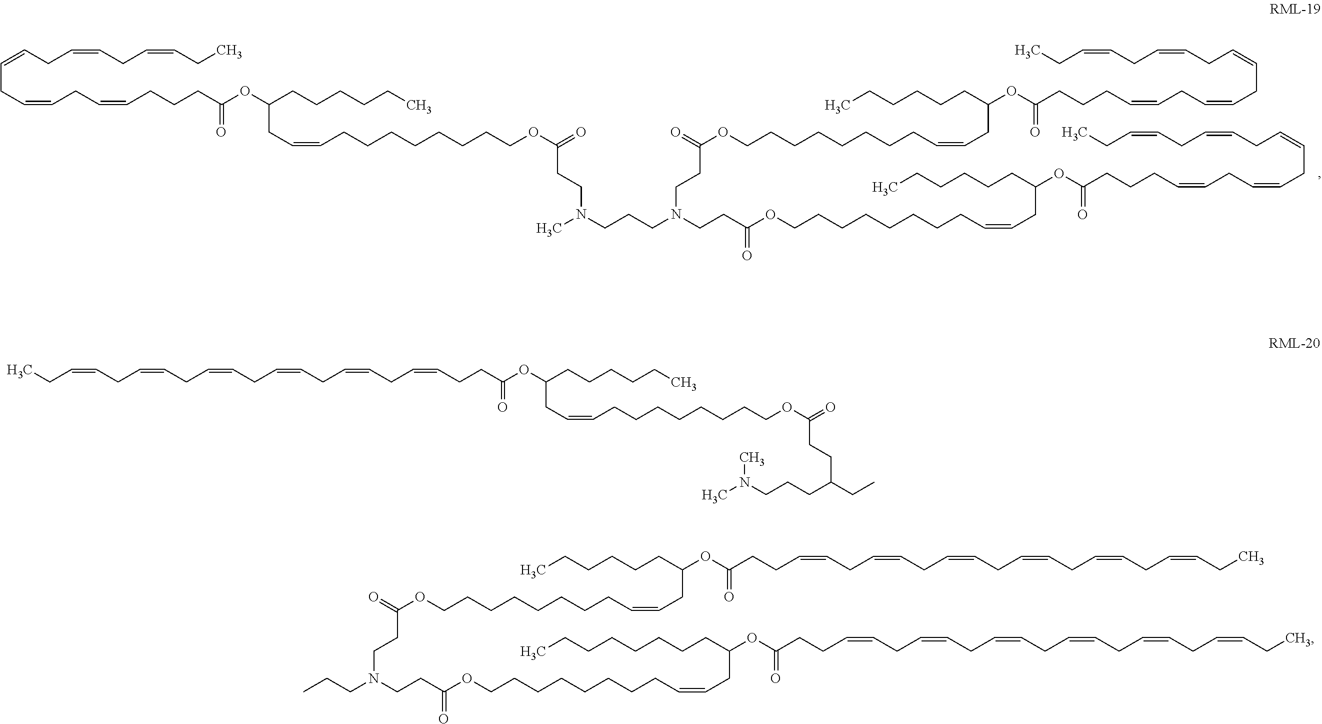 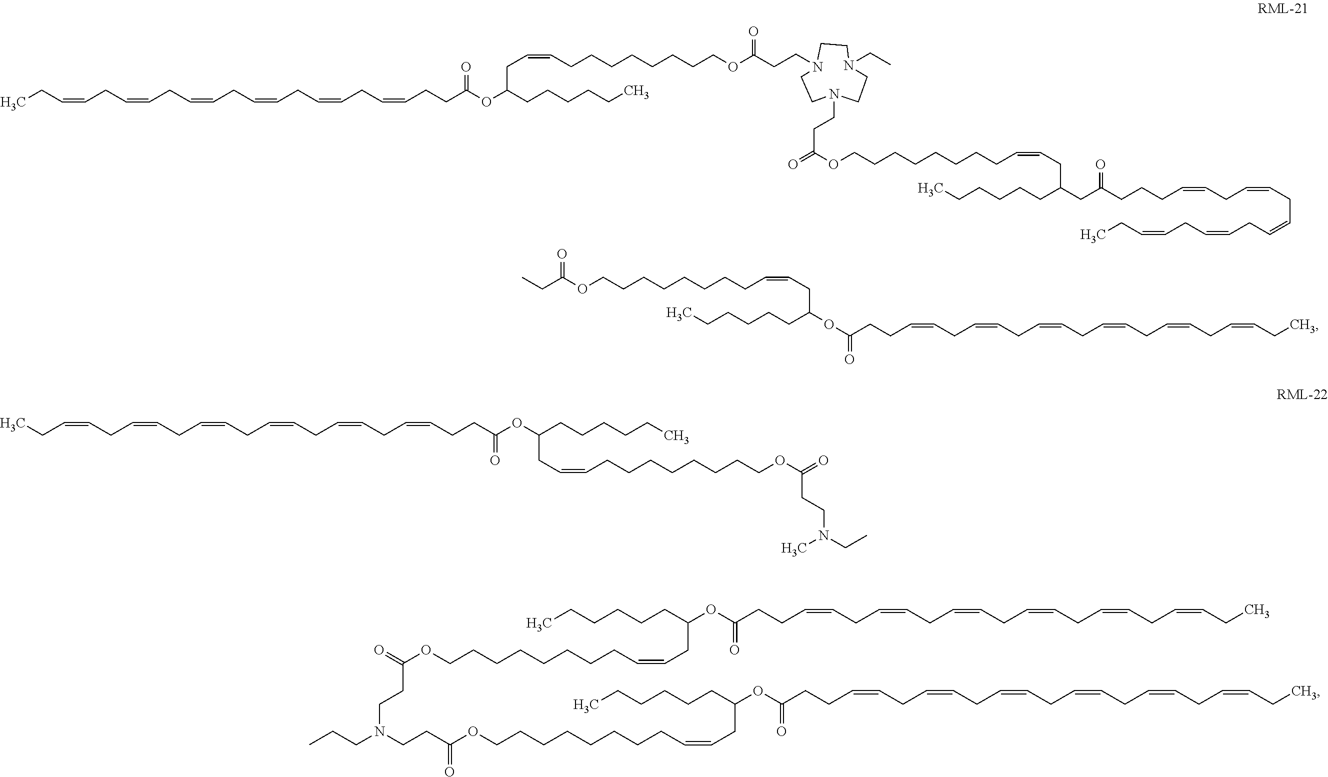 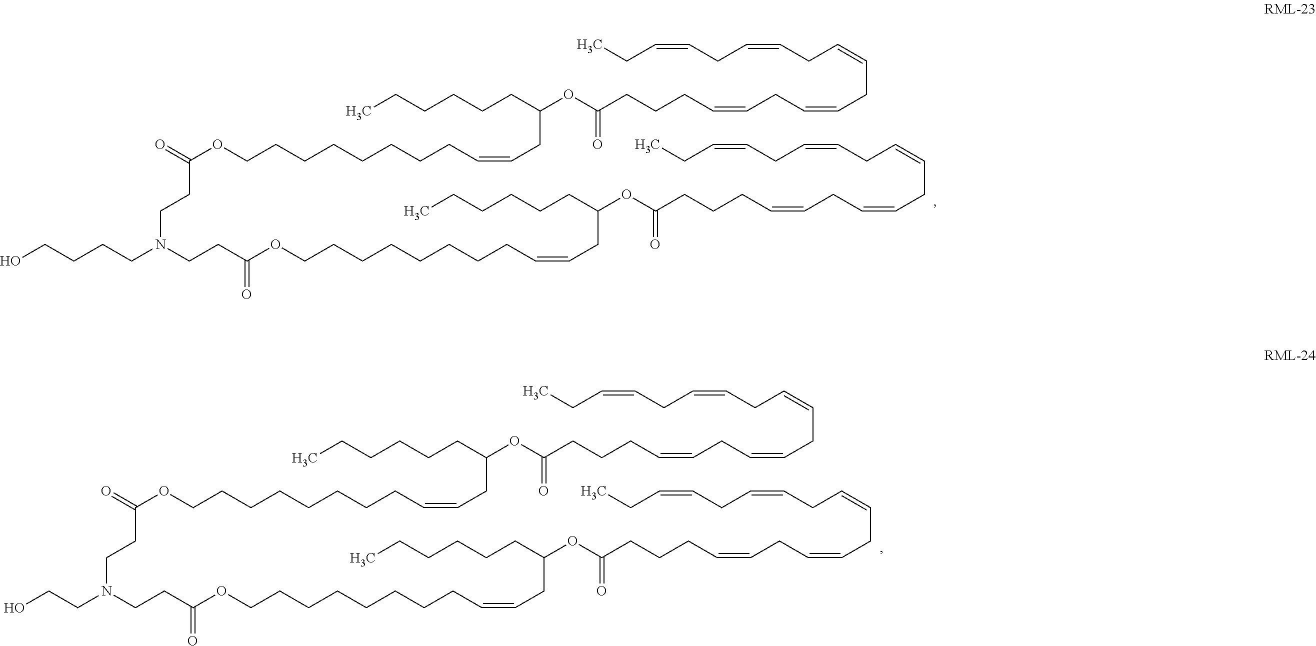 [SMILES: CC/C=C\C/C=C\C/C=C\C/C=C\C/C=C\CCCC(=O)OC(C/C=C\CCCCCCCCOC(=O)CCN(CCCCO)CCC(=O)OCCCCCCCC/C=C\CC(CCCCCC)OC(=O)CCC/C=C\C/C=C\C/C=C\C/C=C\C/C=C\CC)CCCCCC ; CC/C=C\C/C=C\C/C=C\C/C=C\C/C=C\CCCC(=O)OC(C/C=C\CCCCCCCCOC(=O)CCN(CCO)CCC(=O)OCCCCCCCC/C=C\CC(CCCCCC)OC(=O)CCC/C=C\C/C=C\C/C=C\C/C=C\C/C=C\CC)CCCCCC] 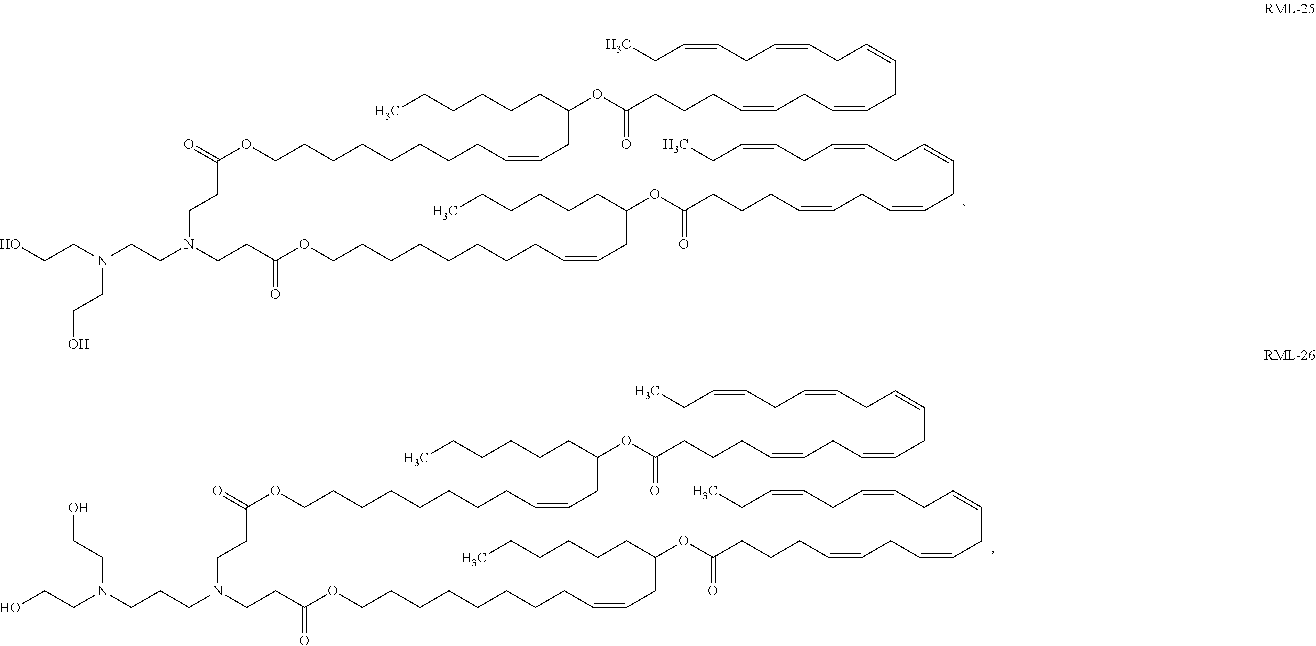 [SMILES: CC/C=C\C/C=C\C/C=C\C/C=C\C/C=C\CCCC(=O)OC(C/C=C\CCCCCCCCOC(=O)CCN(CCC(=O)OCCCCCCCC/C=C\CC(CCCCCC)OC(=O)CCC/C=C\C/C=C\C/C=C\C/C=C\C/C=C\CC)CCN(CCO)CCO)CCCCCC ; CC/C=C\C/C=C\C/C=C\C/C=C\C/C=C\CCCC(=O)OC(C/C=C\CCCCCCCCOC(=O)CCN(CCCN(CCO)CCO)CCC(=O)OCCCCCCCC/C=C\CC(CCCCCC)OC(=O)CCC/C=C\C/C=C\C/C=C\C/C=C\C/C=C\CC)CCCCCC] 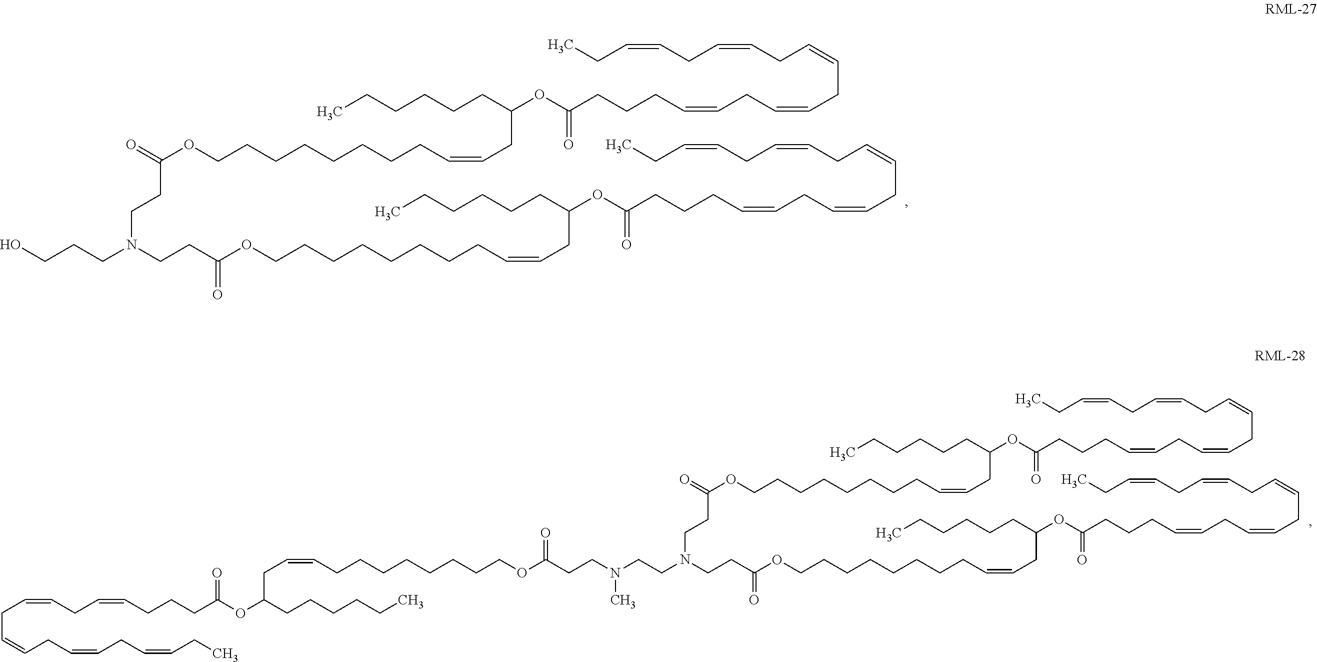 [SMILES: CC/C=C\C/C=C\C/C=C\C/C=C\C/C=C\CCCC(=O)OC(C/C=C\CCCCCCCCOC(=O)CCN(CCCO)CCC(=O)OCCCCCCCC/C=C\CC(CCCCCC)OC(=O)CCC/C=C\C/C=C\C/C=C\C/C=C\C/C=C\CC)CCCCCC ; CC/C=C\C/C=C\C/C=C\C/C=C\C/C=C\CCCC(=O)OC(C/C=C\CCCCCCCCOC(=O)CCN(C)CCN(CCC(=O)OCCCCCCCC/C=C\CC(CCCCCC)OC(=O)CCC/C=C\C/C=C\C/C=C\C/C=C\C/C=C\CC)CCC(=O)OCCCCCCCC/C=C\CC(CCCCCC)OC(=O)CCC/C=C\C/C=C\C/C=C\C/C=C\C/C=C\CC)CCCCCC] 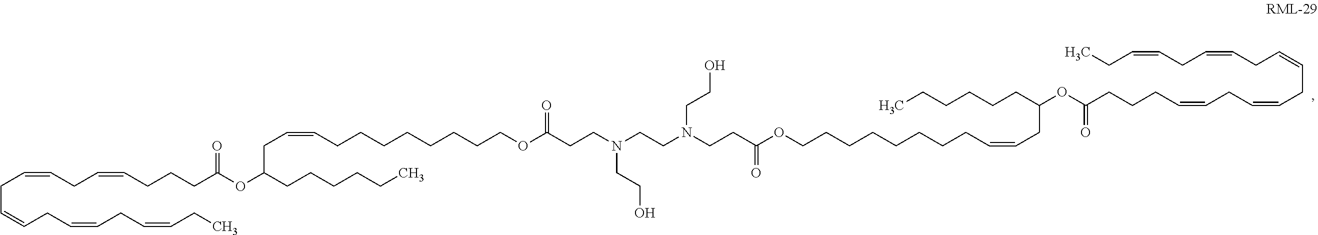 [SMILES: CC/C=C\C/C=C\C/C=C\C/C=C\C/C=C\CCCC(=O)OC(C/C=C\CCCCCCCCOC(=O)CCN(CCO)CCN(CCO)CCC(=O)OCCCCCCCC/C=C\CC(CCCCCC)OC(=O)CCC/C=C\C/C=C\C/C=C\C/C=C\C/C=C\CC)CCCCCC] 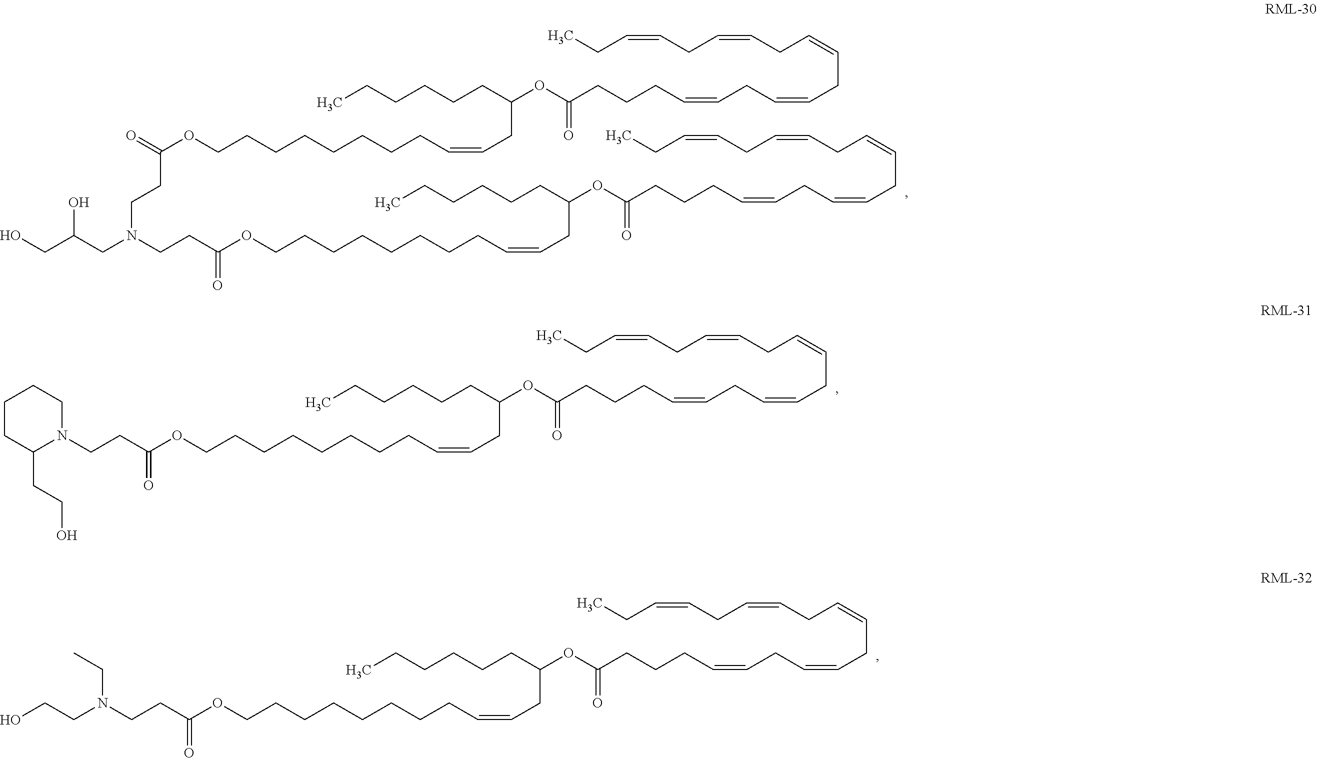 [SMILES: CC/C=C\C/C=C\C/C=C\C/C=C\C/C=C\CCCC(=O)OC(C/C=C\CCCCCCCCOC(=O)CCN(CCC(=O)OCCCCCCCC/C=C\CC(CCCCCC)OC(=O)CCC/C=C\C/C=C\C/C=C\C/C=C\C/C=C\CC)CC(O)CO)CCCCCC ; CC/C=C\C/C=C\C/C=C\C/C=C\C/C=C\CCCC(=O)OC(C/C=C\CCCCCCCCOC(=O)CCN1CCCCC1CCO)CCCCCC ; CC/C=C\C/C=C\C/C=C\C/C=C\C/C=C\CCCC(=O)OC(C/C=C\CCCCCCCCOC(=O)CCN(CC)CCO)CCCCCC] 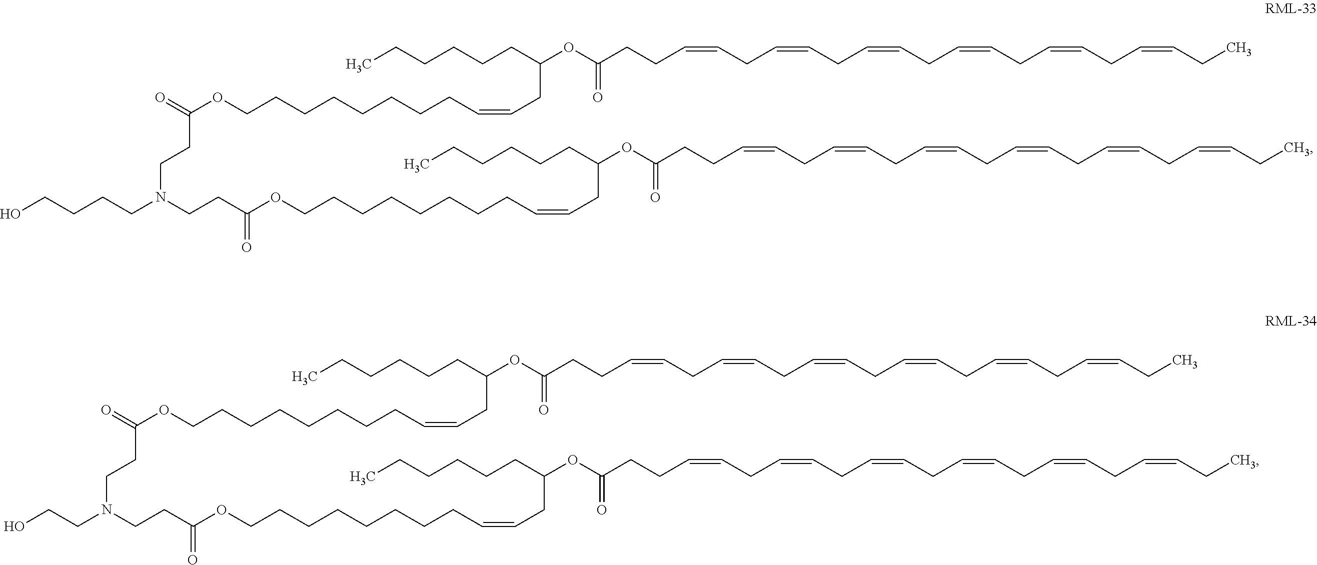 [SMILES: CC/C=C\C/C=C\C/C=C\C/C=C\C/C=C\C/C=C\CCC(=O)OC(C/C=C\CCCCCCCCOC(=O)CCN(CCCCO)CCC(=O)OCCCCCCCC/C=C\CC(CCCCCC)OC(=O)CC/C=C\C/C=C\C/C=C\C/C=C\C/C=C\C/C=C\CC)CCCCCC ; CC/C=C\C/C=C\C/C=C\C/C=C\C/C=C\C/C=C\CCC(=O)OC(C/C=C\CCCCCCCCOC(=O)CCN(CCO)CCC(=O)OCCCCCCCC/C=C\CC(CCCCCC)OC(=O)CC/C=C\C/C=C\C/C=C\C/C=C\C/C=C\C/C=C\CC)CCCCCC] 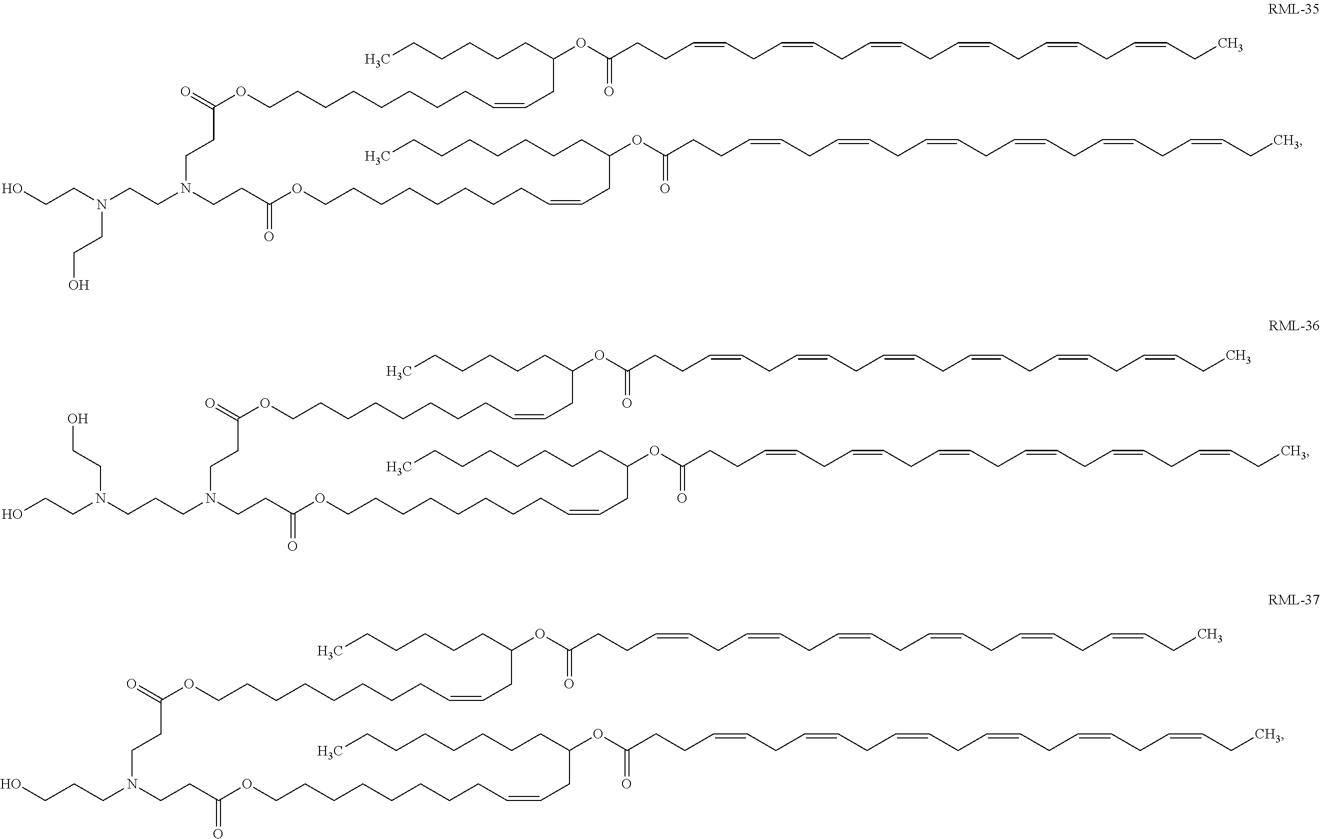 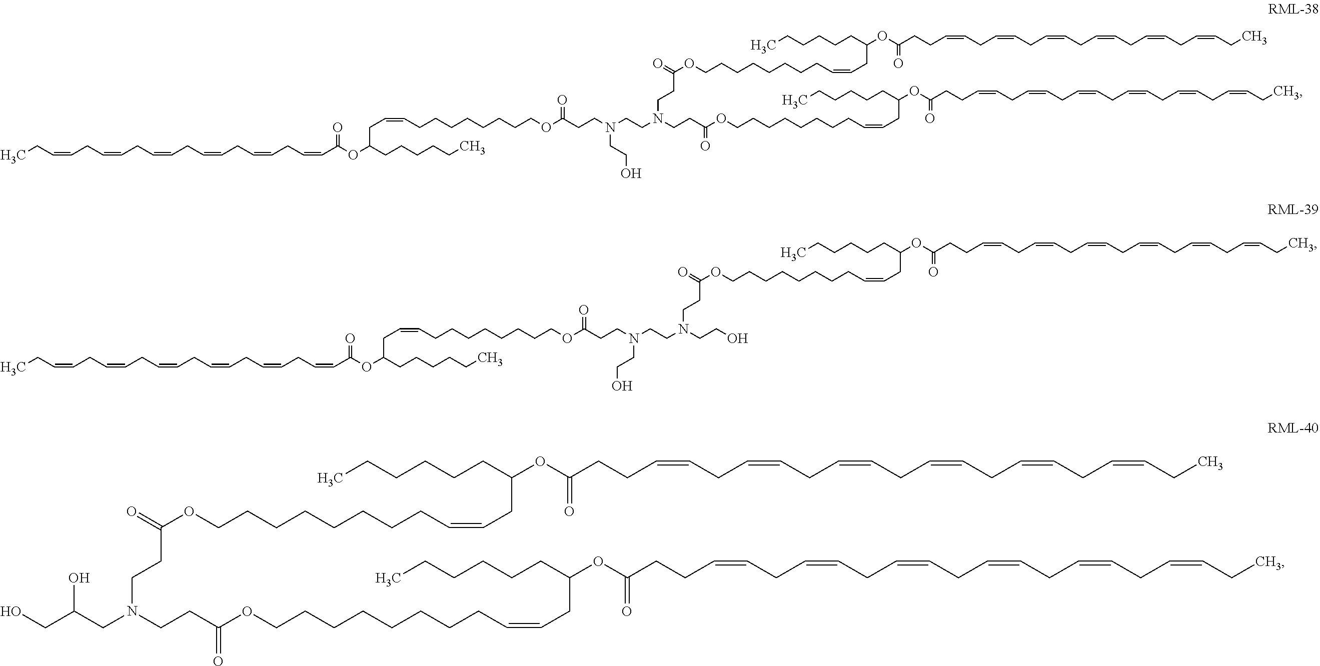 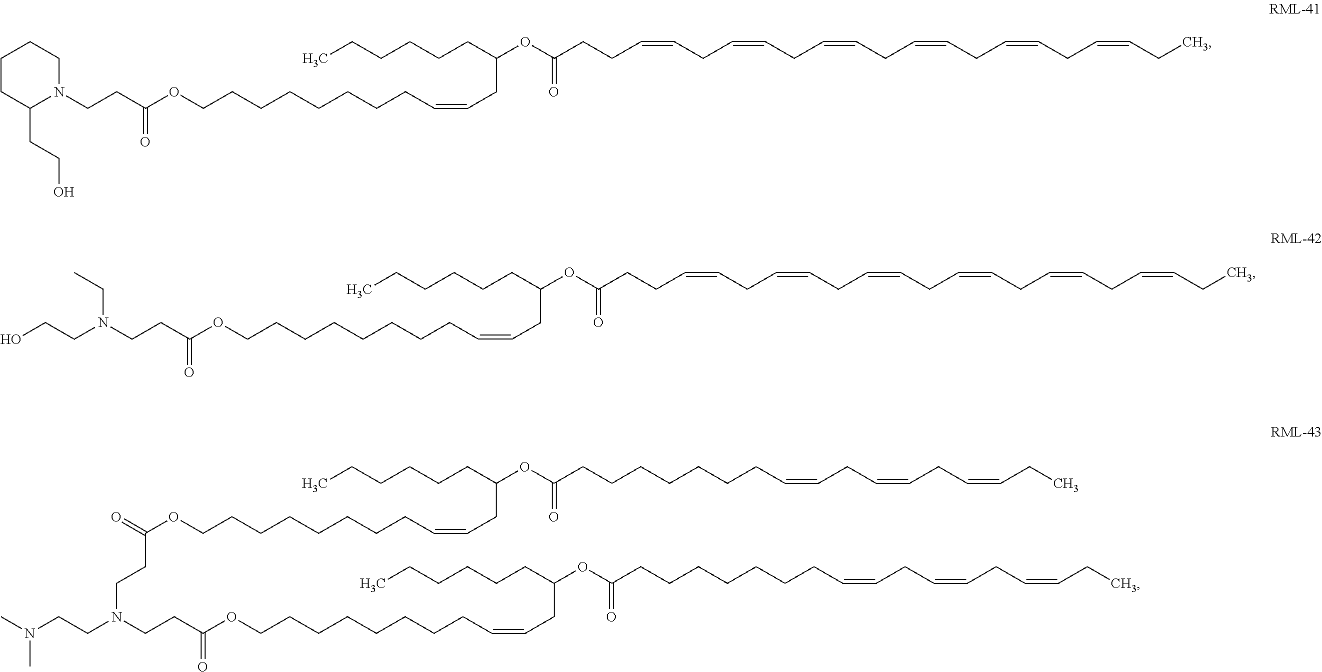 [SMILES: CC/C=C\C/C=C\C/C=C\C/C=C\C/C=C\C/C=C\CCC(=O)OC(C/C=C\CCCCCCCCOC(=O)CCN1CCCCC1CCO)CCCCCC ; CC/C=C\C/C=C\C/C=C\C/C=C\C/C=C\C/C=C\CCC(=O)OC(C/C=C\CCCCCCCCOC(=O)CCN(CC)CCO)CCCCCC ; CC/C=C\C/C=C\C/C=C\CCCCCCCC(=O)OC(C/C=C\CCCCCCCCOC(=O)CCN(CCC(=O)OCCCCCCCC/C=C\CC(CCCCCC)OC(=O)CCCCCCC/C=C\C/C=C\C/C=C\CC)CCN(C)C)CCCCCC] 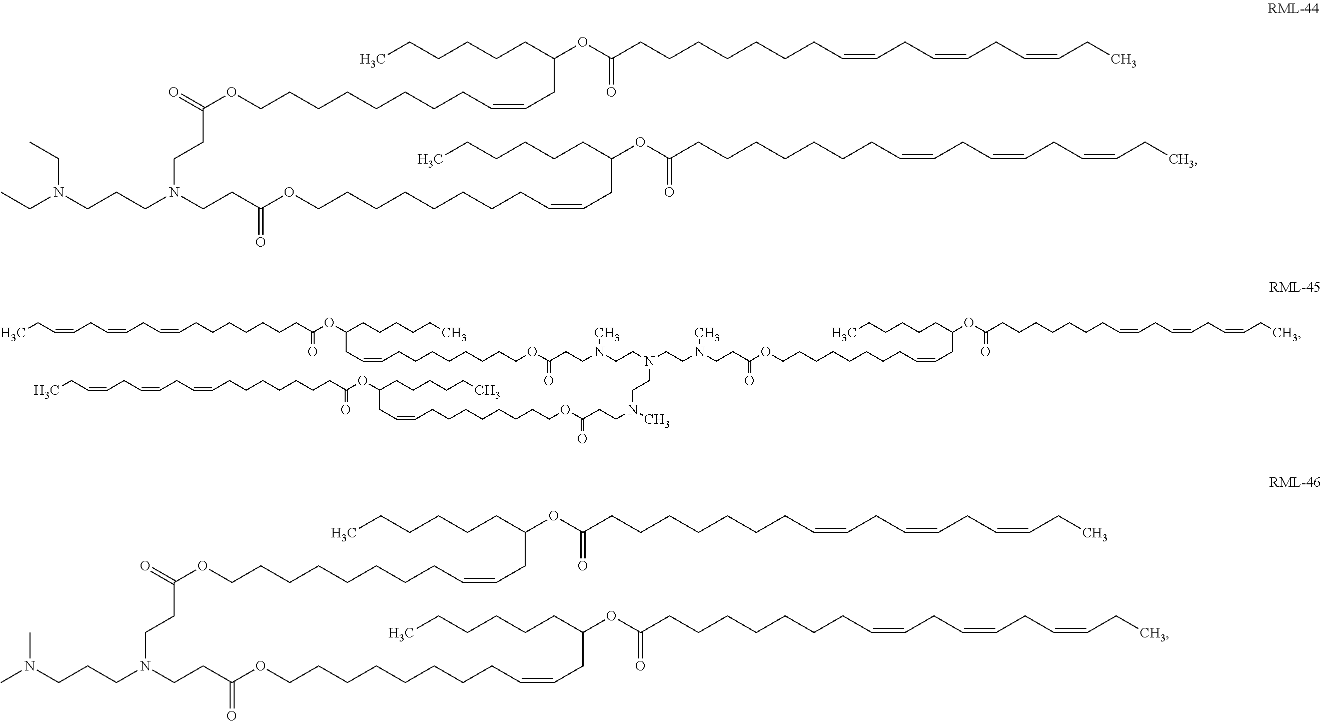 [SMILES: CC/C=C\C/C=C\C/C=C\CCCCCCCC(=O)OC(C/C=C\CCCCCCCCOC(=O)CCN(CCCN(CC)CC)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCC(=O)OC(C/C=C\CCCCCCCCOC(=O)CCN(C)CCN(CCN(C)CCC(=O)OCCCCCCCC/C=C\CC(CCCCCC)OC(=O)CCCCCCC/C=C\C/C=C\C/C=C\CC)CCN(C)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCC(=O)OC(C/C=C\CCCCCCCCOC(=O)CCN(CCCN(C)C)CCC(=O)OCCCCCCCC/C=C\CC(CCCCCC)OC(=O)CCCCCCC/C=C\C/C=C\C/C=C\CC)CCCCCC] 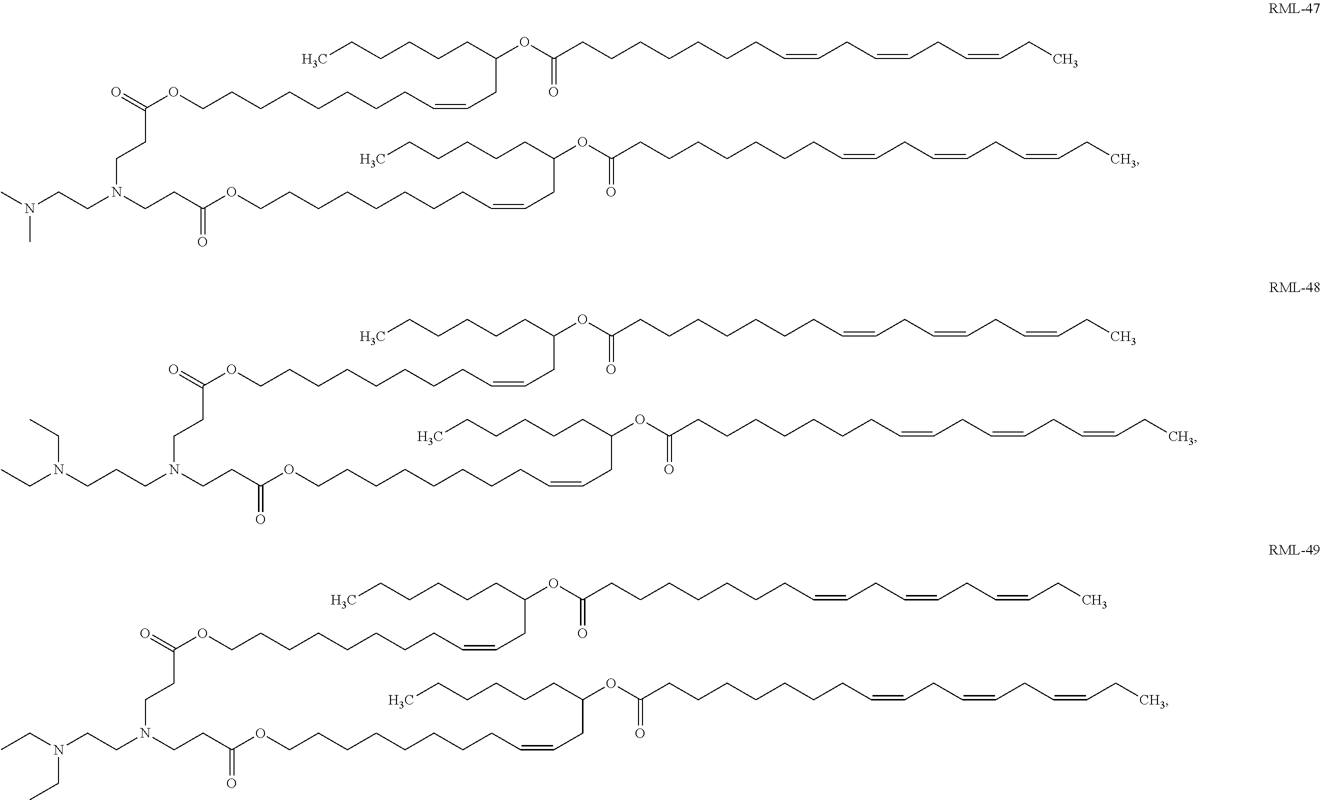 [SMILES: CC/C=C\C/C=C\C/C=C\CCCCCCCC(=O)OC(C/C=C\CCCCCCCCOC(=O)CCN(CCC(=O)OCCCCCCCC/C=C\CC(CCCCCC)OC(=O)CCCCCCC/C=C\C/C=C\C/C=C\CC)CCN(C)C)CCCCCC ; CC/C=C\C/C=C\C/C=C\CCCCCCCC(=O)OC(C/C=C\CCCCCCCCOC(=O)CCN(CCCN(CC)CC)CCC(=O)OCCCCCCCC/C=C\CC(CCCCCC)OC(=O)CCCCCCC/C=C\C/C=C\C/C=C\CC)CCCCCC ; CC/C=C\C/C=C\C/C=C\CCCCCCCC(=O)OC(C/C=C\CCCCCCCCOC(=O)CCN(CCC(=O)OCCCCCCCC/C=C\CC(CCCCCC)OC(=O)CCCCCCC/C=C\C/C=C\C/C=C\CC)CCN(CC)CC)CCCCCC] 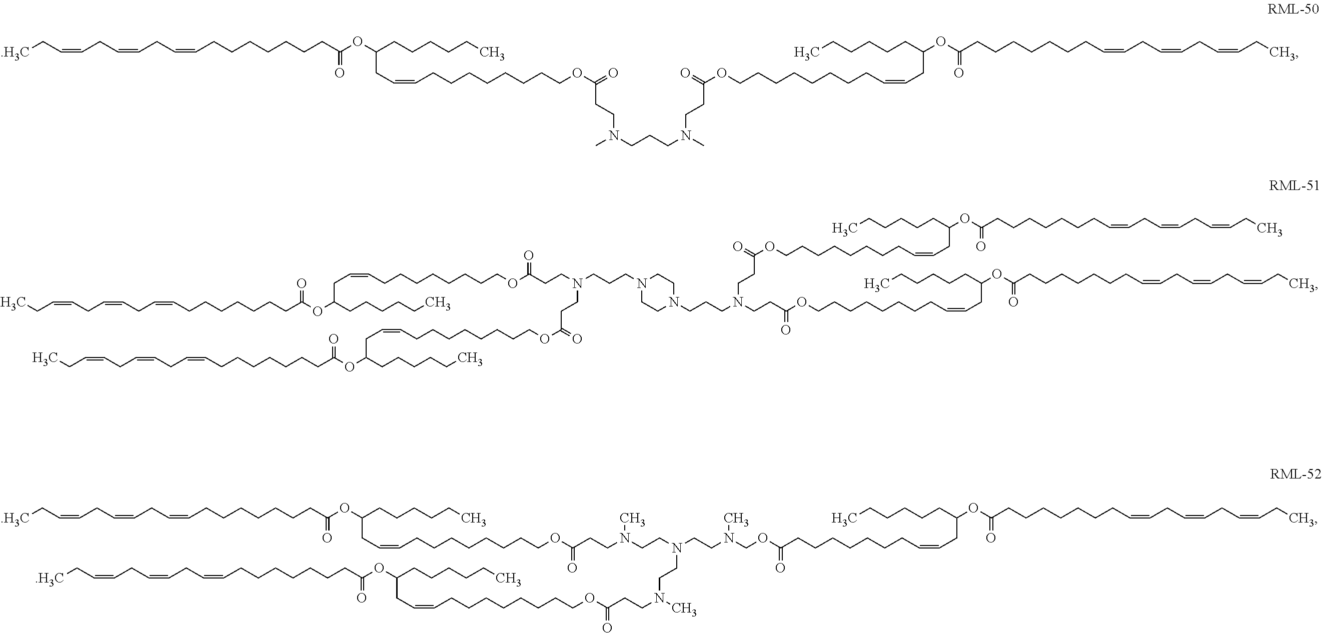 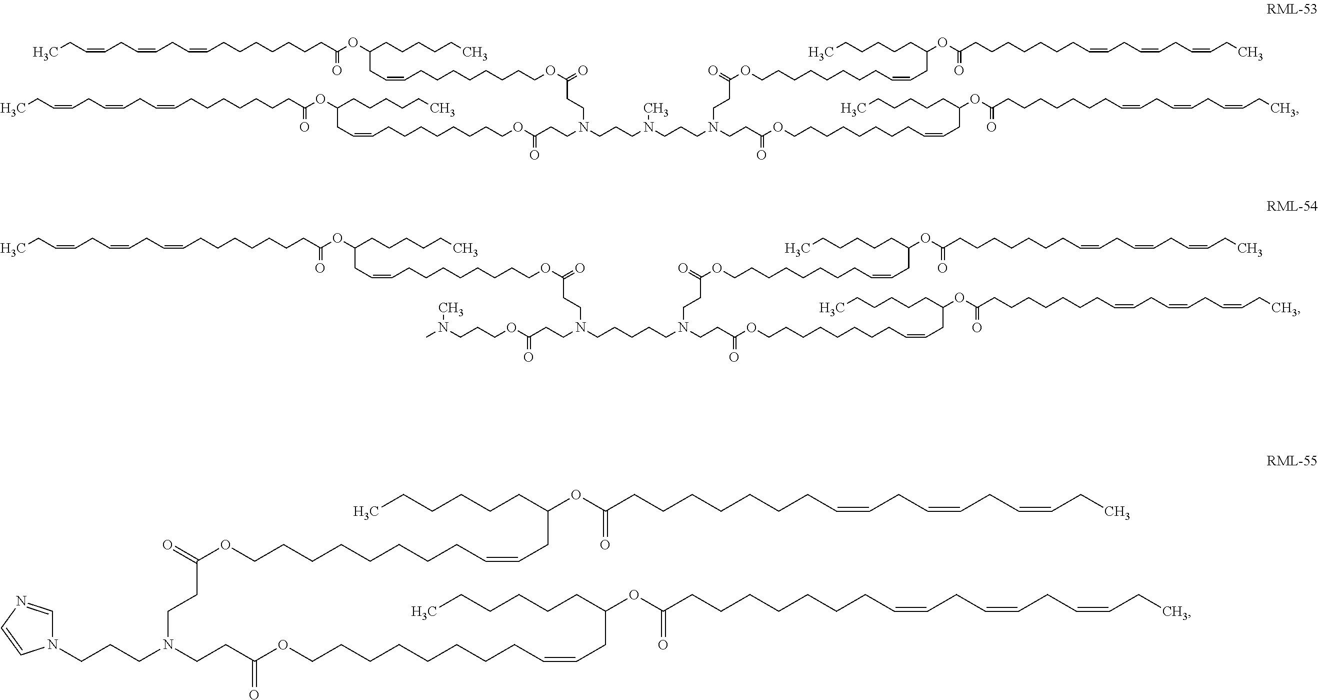 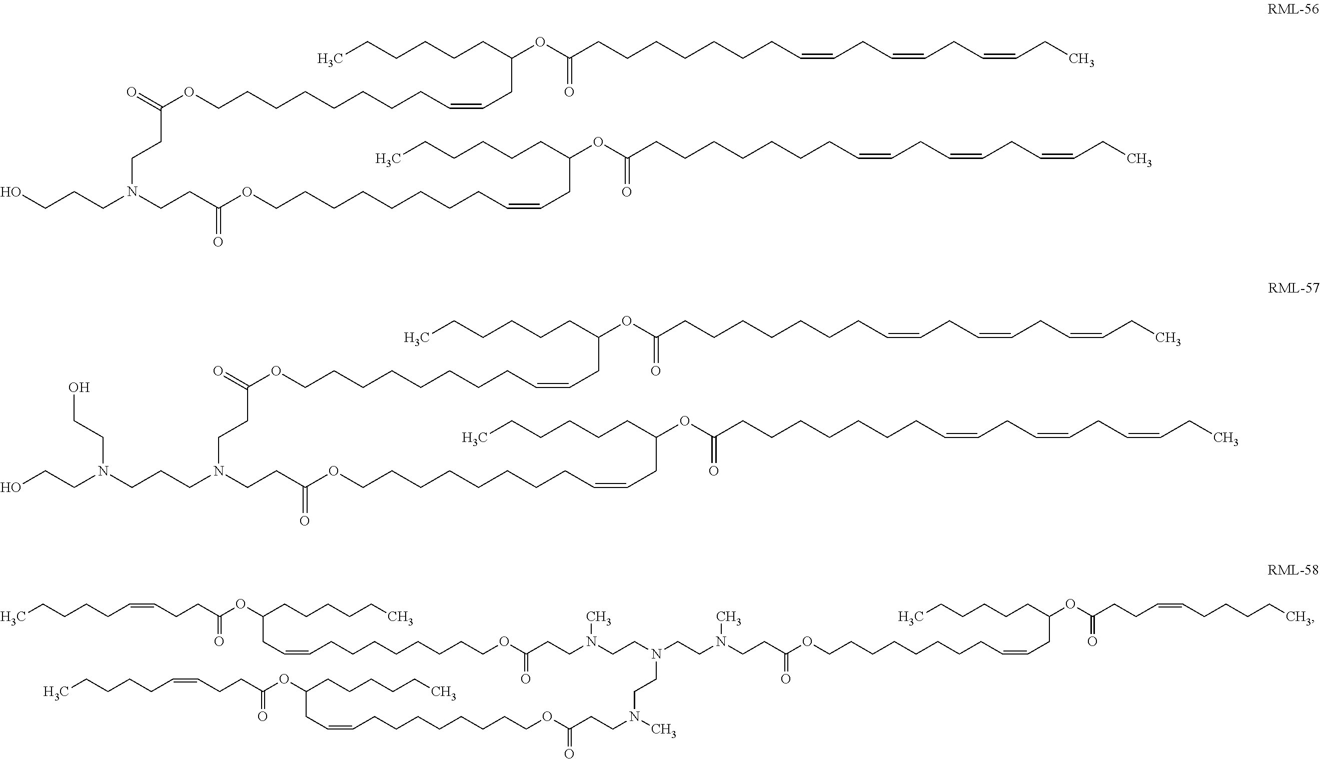 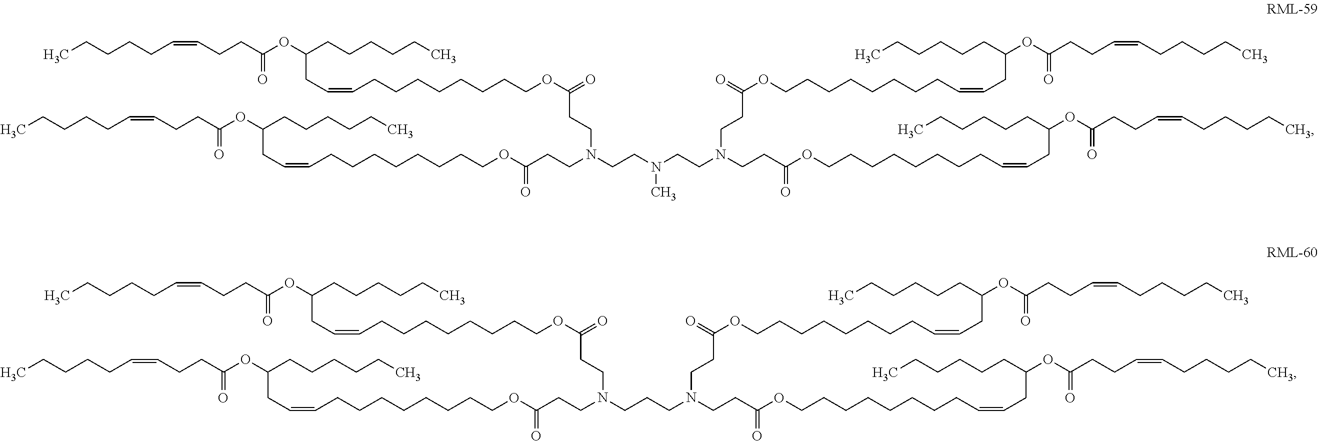 [SMILES: CCCCC/C=C\CCC(=O)OC(C/C=C\CCCCCCCCOC(=O)CCN(CCC(=O)OCCCCCCCC/C=C\CC(CCCCCC)OC(=O)CC/C=C\CCCCC)CCN(C)CCN(CCC(=O)OCCCCCCCC/C=C\CC(CCCCCC)OC(=O)CC/C=C\CCCCC)CCC(=O)OCCCCCCCC/C=C\CC(CCCCCC)OC(=O)CC/C=C\CCCCC)CCCCCC ; CCCCC/C=C\CCC(=O)OC(C/C=C\CCCCCCCCOC(=O)CCN(CCCN(CCC(=O)OCCCCCCCC/C=C\CC(CCCCCC)OC(=O)CC/C=C\CCCCC)CCC(=O)OCCCCCCCC/C=C\CC(CCCCCC)OC(=O)CC/C=C\CCCCC)CCC(=O)OCCCCCCCC/C=C\CC(CCCCCC)OC(=O)CC/C=C\CCCCC)CCCCCC] 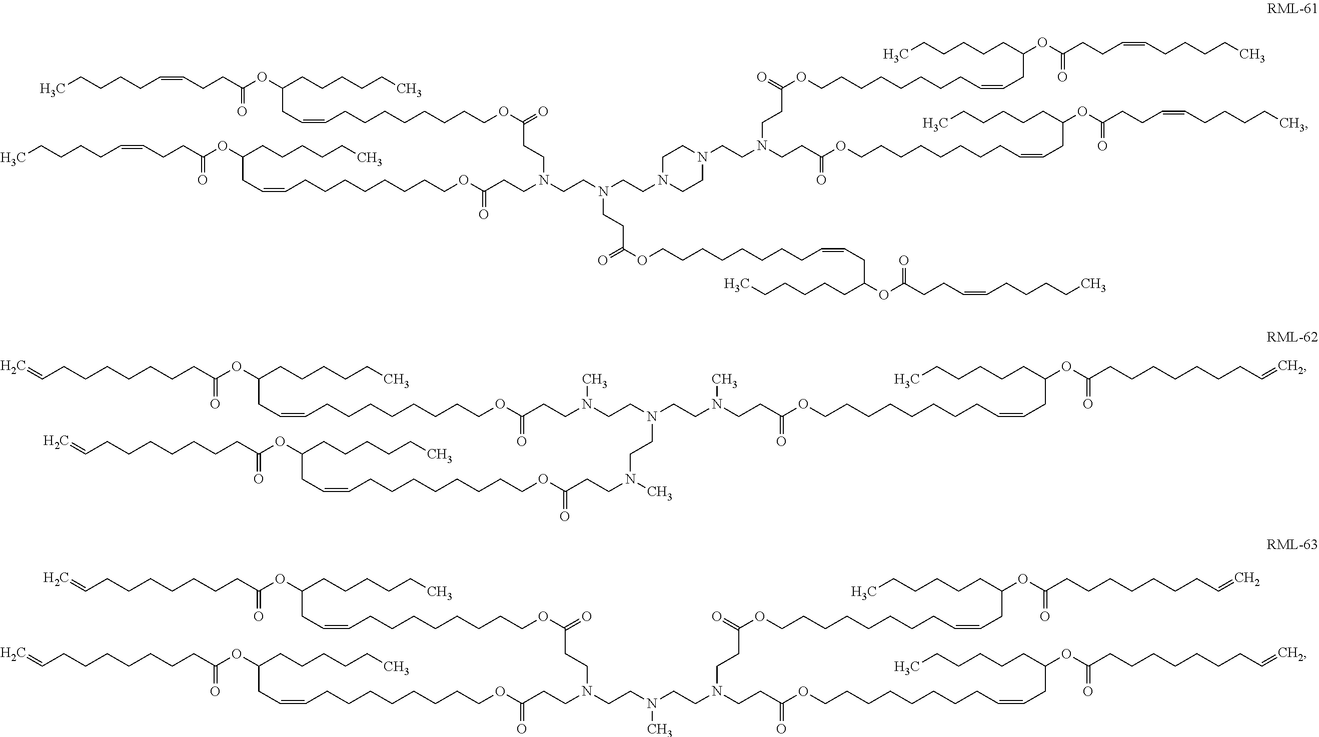 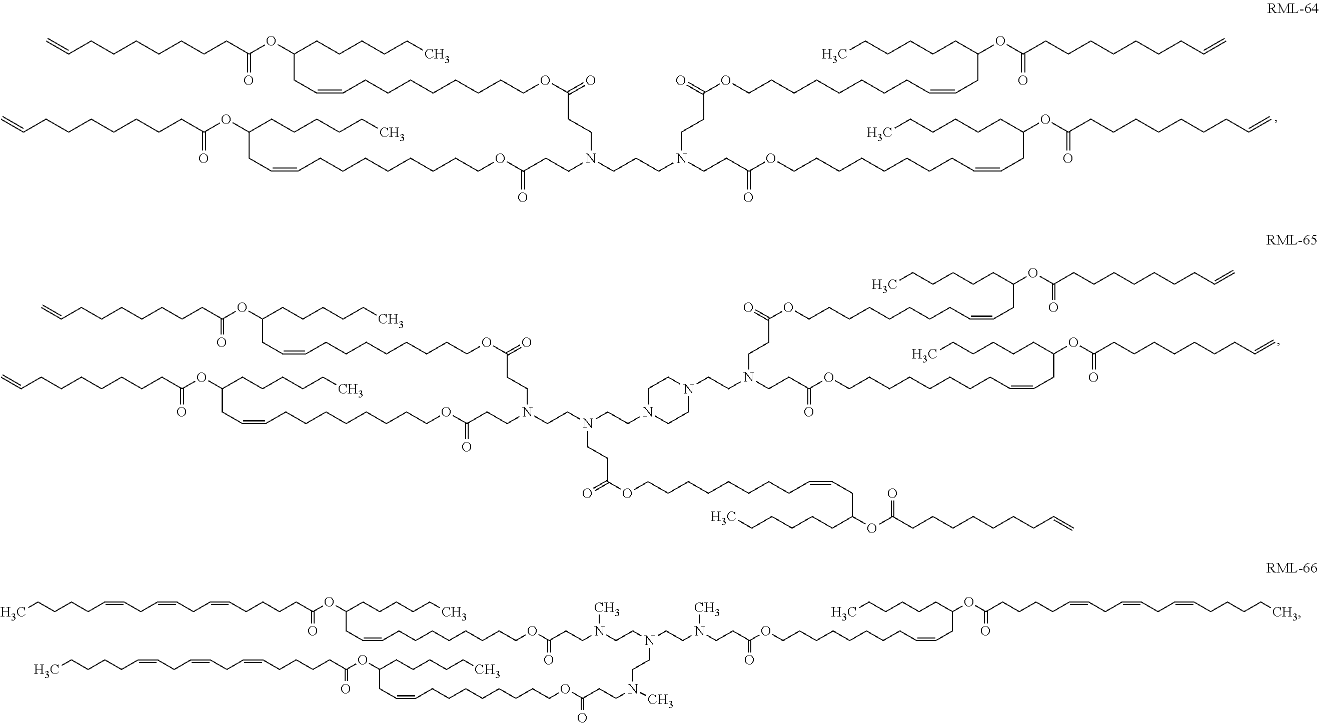 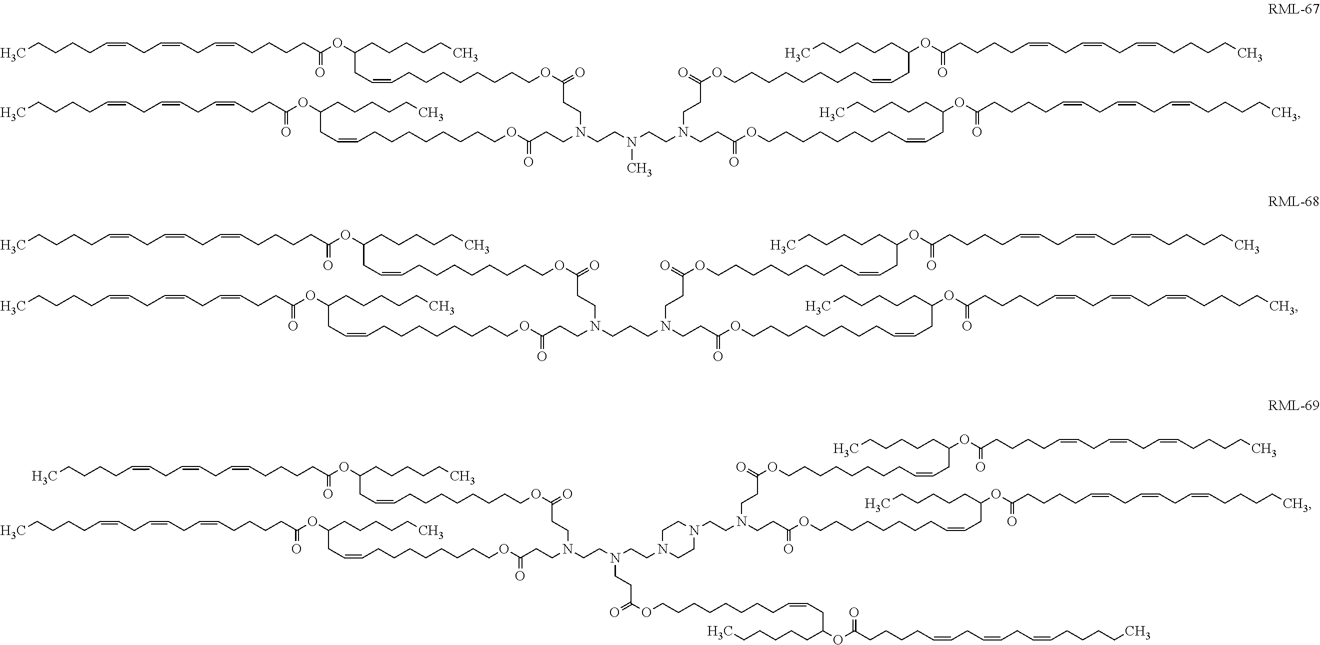 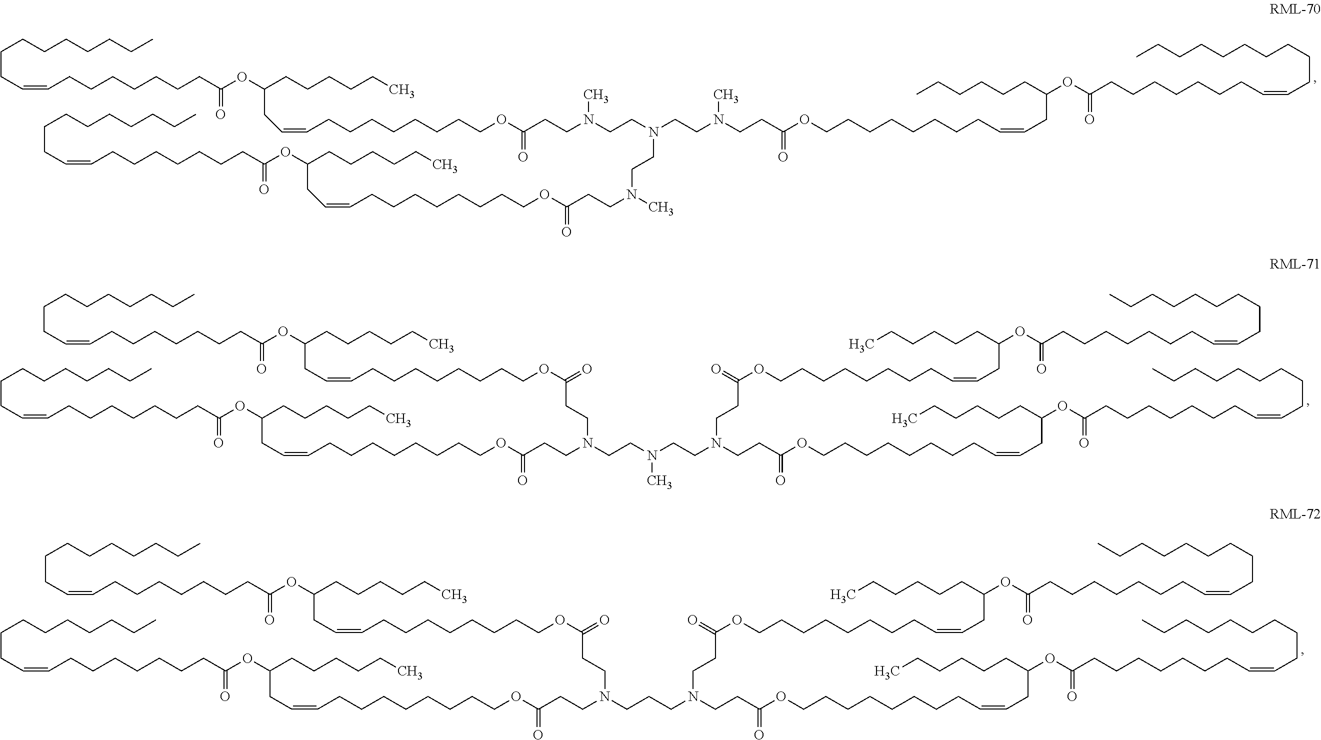 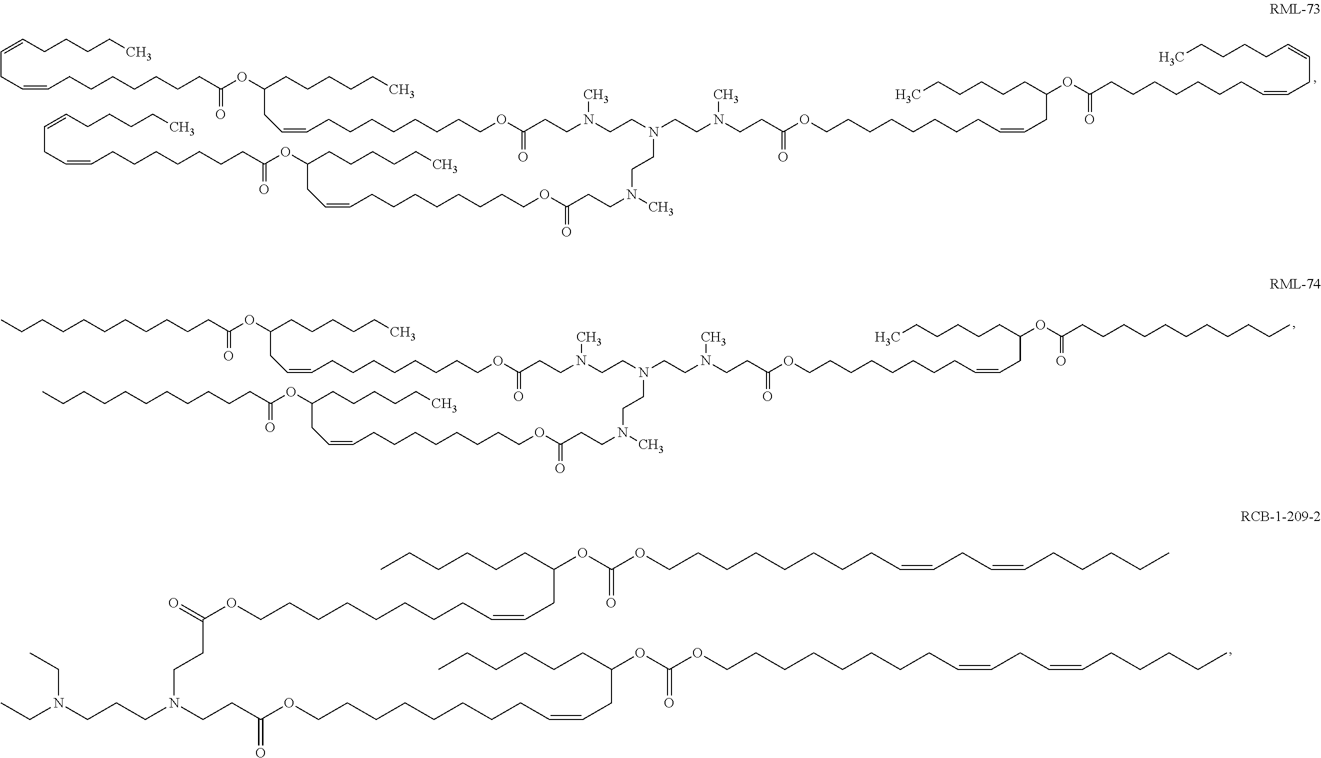 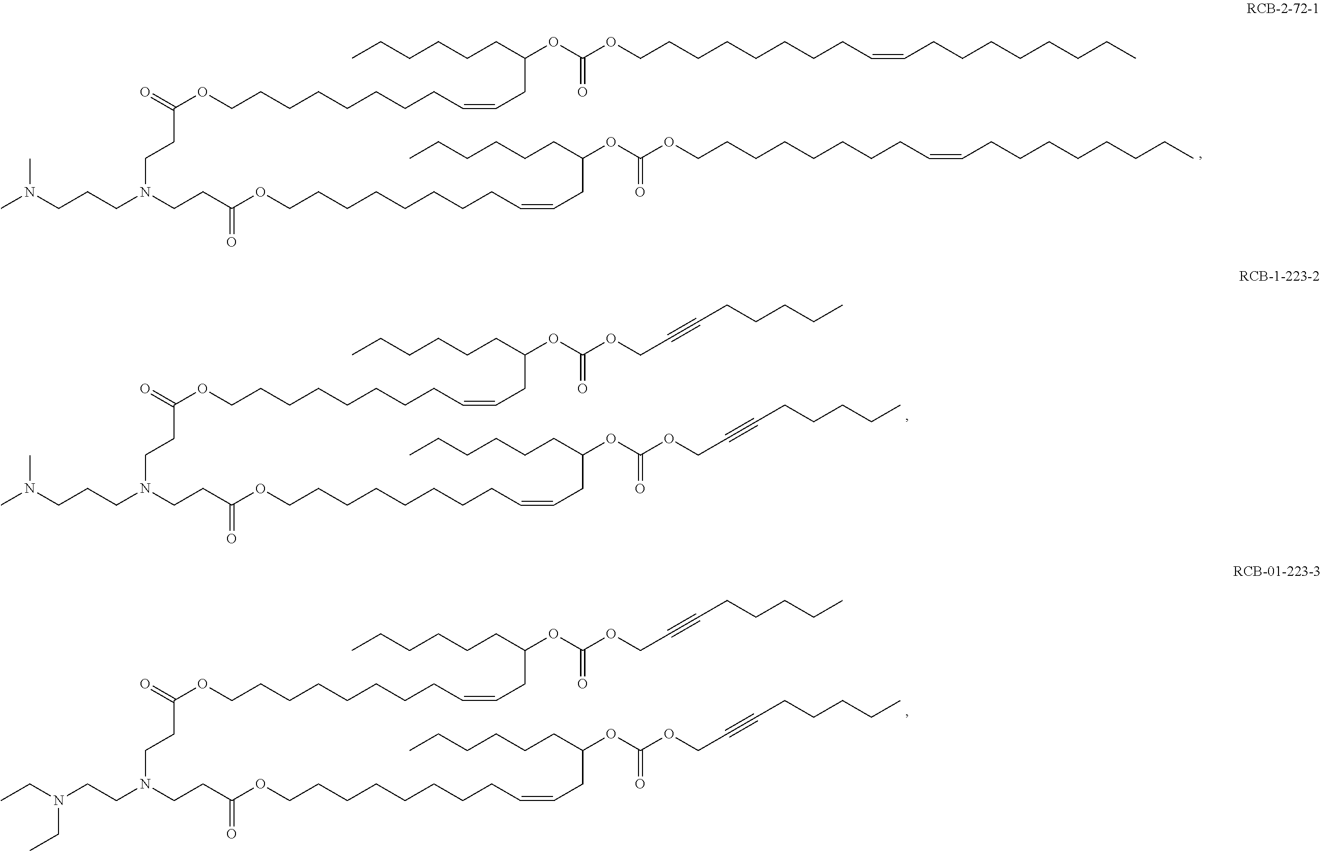 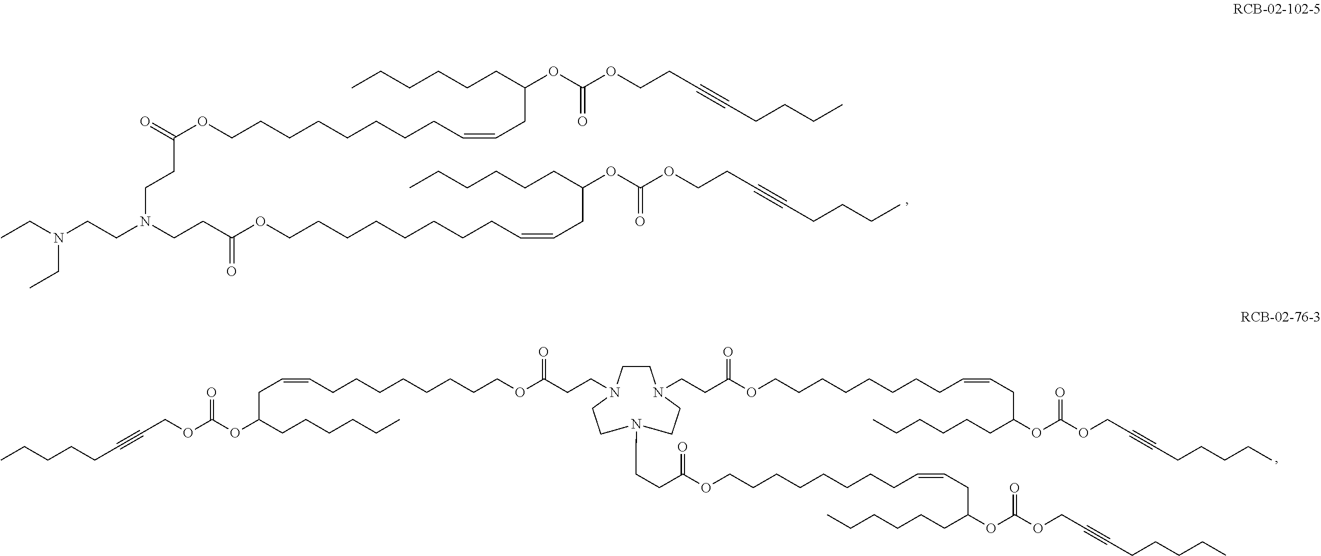 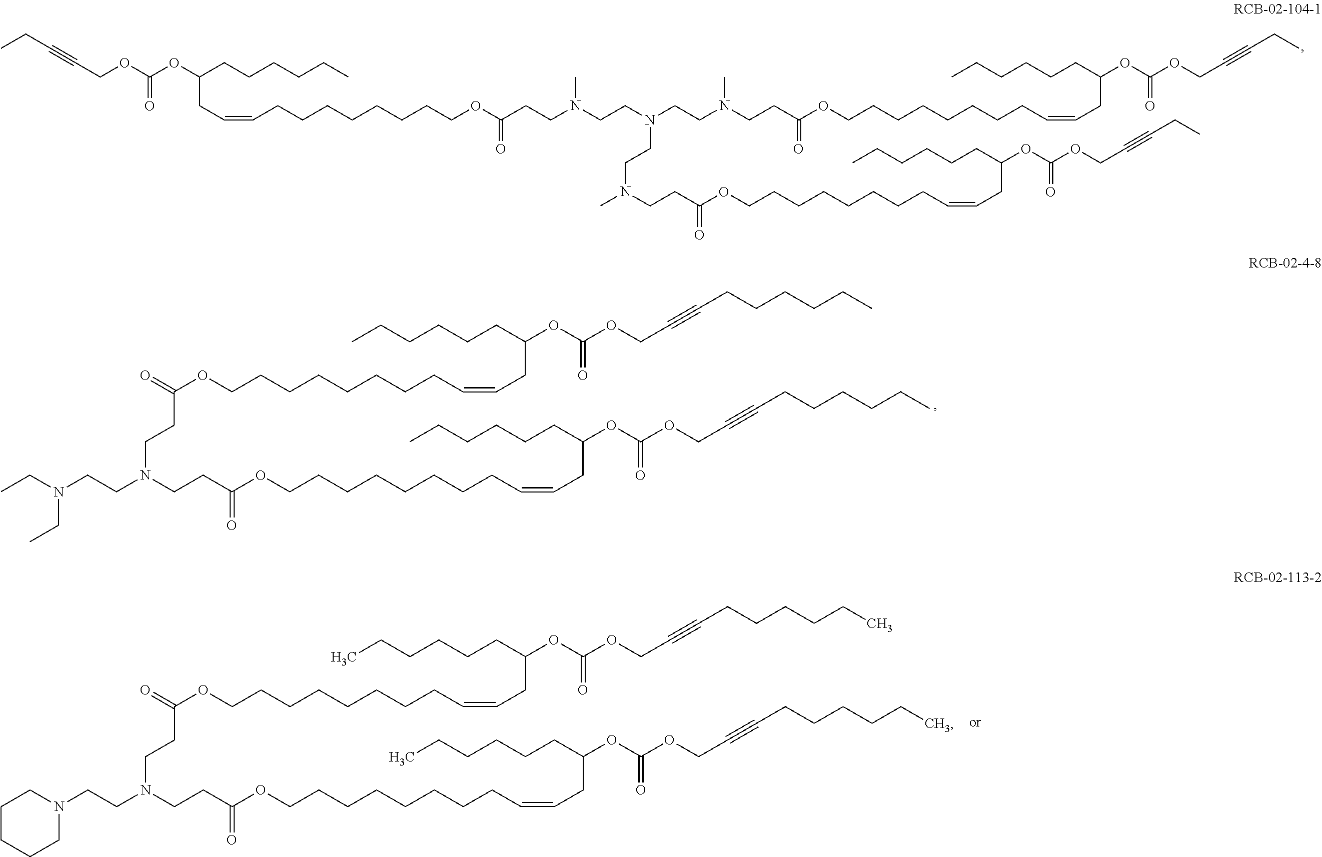 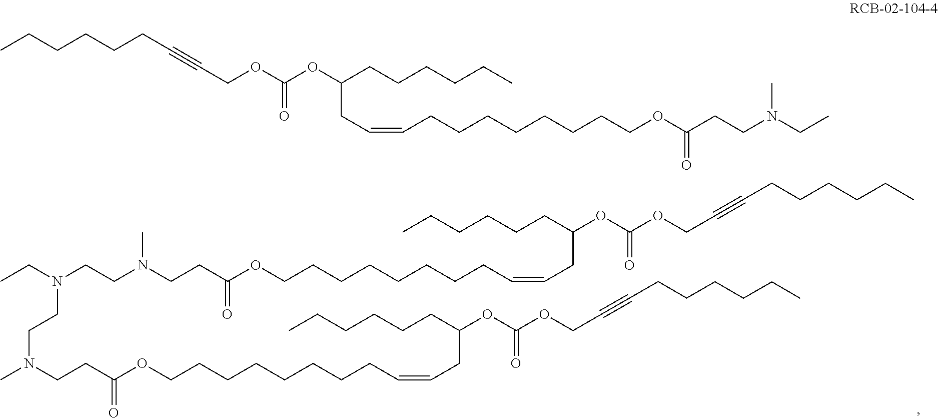
or a pharmaceutically acceptable salt, solvate, tautomer, stereoisomer, or isotopically labeled derivative thereof.

26. A pharmaceutical composition comprising a compound of any one of the preceding claims, or a pharmaceutically acceptable salt, solvate, tautomer, stereoisomer, or isotopically labeled derivative thereof, and a pharmaceutically acceptable excipient.

27. The pharmaceutical composition of claim 26, wherein the pharmaceutical composition further comprises an agent.

28. The pharmaceutical composition of claim 27, wherein the agent is an organic molecule, inorganic molecule, polynucleotide, protein, peptide, polynucleotide, targeting agent, an isotopically labeled chemical compound, vaccine, an immunological agent, or an agent useful in bioprocessing.

29. The pharmaceutical composition of claim 27 or 28, wherein the agent is a polynucleotide.

30. The pharmaceutical composition of claim 29, wherein the polynucleotide is an RNA.

31. The pharmaceutical composition of claim 30, wherein the RNA is messenger RNA (mRNA), single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA.

32. The pharmaceutical composition of claim 31, wherein the agent is mRNA.

33. The pharmaceutical composition of claim 29, wherein the polynucleotide is a DNA.

34. The pharmaceutical composition of claim 33, wherein the DNA is a plasmid DNA (pDNA).

35. The pharmaceutical composition of any one of claims 26-34, wherein the pharmaceutical composition further comprises one or more of a PEG-lipid, sterol, phospholipid, or charged lipid.

36. The pharmaceutical composition of any one of claims 27-35, wherein the agent and the compound, or the pharmaceutically acceptable salt, solvate, tautomer, stereoisomer, or isotopically labeled derivative thereof, are not covalently attached.

37. The pharmaceutical composition of any one of claims 26-36, wherein the composition is in the form of a particle.

38. The pharmaceutical composition of claim 37, wherein the particle is a nanoparticle or microparticle.

39. The pharmaceutical composition of claim 37, wherein the particle is a micelle, liposome, or lipoplex.

40. The pharmaceutical composition of claim 37, wherein the particle encapsulates the agent.

41. A method of delivering a polynucleotide to a subject, comprising administering to the subject a composition comprising a polynucleotide and a compound of any one of claims 1-22.

42. The method of claim 41, wherein the polynucleotide is delivered to the lungs, liver, or spleen of the subject.

43. The method of claim 42, wherein the polynucleotide is delivered to the lungs of the subject.

44. The method of any one of claims 41-43, wherein the composition is administered by inhalation, intravenously, intratracheally, or intramuscularly.

45. A method of treating or preventing a disease, disorder, or condition in a subject, comprising administering to the subject a composition comprising an agent and a compound of any one of claims 1-25.

46. The method of claim 45, wherein the disease, disorder, or condition is a genetic disease, proliferative disease, hematological disease, neurological disease, liver disease, spleen disease, lung disease, painful condition, psychiatric disorder, musculoskeletal disease, a metabolic disorder, inflammatory disease, or autoimmune disease.

47. The method of claim 45 or 46, wherein the disease, disorder, or condition is a lung disease.

48. The method of any one of claims 45-47, wherein the agent is an organic molecule, inorganic molecule, polynucleotide, protein, peptide, polynucleotide, targeting agent, an isotopically labeled chemical compound, vaccine, an immunological agent, or an agent useful in bioprocessing.

49. The method of any one of claims 45-48 wherein the agent is a polynucleotide.

50. The method of claim 49, wherein the polynucleotide is an RNA.

51. The method of claim 50, wherein the RNA is messenger RNA (mRNA), single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA.

52. The method of claim 51, wherein the agent is mRNA.

53. The method of claim 49, wherein the polynucleotide is a DNA.

54. The method of claim 53, wherein the DNA is a plasmid DNA (pDNA).

55. A kit comprising:
a compound of any one of claims 1-25, or a pharmaceutically acceptable salt, solvate, tautomer, stereoisomer, or isotopically labeled derivative thereof; or a pharmaceutical composition of any one of claims 26-40; and
instructions for using the compound, or pharmaceutically acceptable salt, solvate, tautomer, stereoisomer, or isotopically labeled derivative thereof, or pharmaceutical composition.

56. A method of preparing a compound of Formula (II), the method comprising reacting a compound of Formula (III): 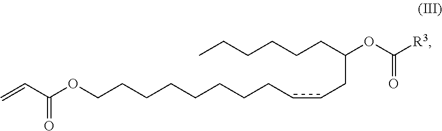
or a salt thereof, with a compound of formula:
H—X,
or a salt thereof, wherein:
R³ is optionally substituted aliphatic or optionally substituted heteroaliphatic;
X is —OR¹, —SR¹, or —NR¹R²;
R¹ is —H, optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl; and 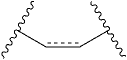
represents either a single or a double bond.

57. The method of claim 56, wherein R³ is C₁-C₂₅ optionally substituted aliphatic, —OR^{3O}, or —N(R^{3N})₂, wherein each instance of R^{3O} and R^{3N} is independently optionally substituted C₁-C₂₅ aliphatic.

58. A compound prepared by reacting a compound of Formula (III): 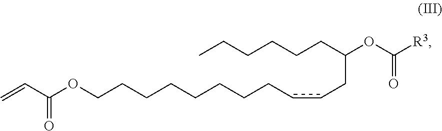
or a salt thereof, with a compound of formula:
H—X,
or a salt thereof, wherein:
R³ is optionally substituted aliphatic or optionally substituted heteroaliphatic;
X is —OR¹, —SR¹, or —NR¹R²;
R¹ is optionally substituted alkyl, optionally substituted heteroalkyl, optionally substituted aryl, optionally substituted heteroaryl, optionally substituted cycloalkyl, optionally substituted heterocyclyl, or a protecting group;
R² is —H, optionally substituted alkyl, optionally substituted heteroalkyl, optionally substituted aryl, optionally substituted heteroaryl, optionally substituted cycloalkyl, optionally substituted heterocyclyl, or a protecting group; or
R¹ and R² are joined together with the intervening atoms to form an optionally substituted heterocyclyl; and 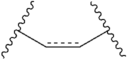
represents either a single or a double bond.

59. The compound of claim 58, wherein R³ is C₁-C₂₅ optionally substituted aliphatic, —OR^{3O}, or —N(R^{3N})₂, wherein each instance of R^{3O} and R^{3N} is independently optionally substituted C₁-C₂₅ aliphatic.

60. An inhalable dry powder composition comprising microparticles, wherein the microparticles comprise a polynucleotide, a lipid nanoparticle, and a saccharide excipient, and wherein at least about 50% of the microparticles in the composition have an aerodynamic size of about 1 micron to about 5 microns, a physical diameter of about 3 microns to about 10 microns, or a combination thereof.

61. The composition of claim 60, wherein the polynucleotide is encapsulated by the lipid nanoparticle.

62. The composition of claim 60 or 61, wherein at least about 70% of the microparticles in the composition have an aerodynamic size of about 1 micron to about 5 microns.

63. The composition of any one of claims 60-62, wherein at least about 70% of the microparticles in the composition have a physical diameter of between about 3 microns and about 10 microns.

64. The composition of any one of claims 60-63, wherein the polynucleotide is at least about 30 nucleotides in length.

65. The composition of claim 64, wherein the polynucleotide is at least about 1,000 nucleotides in length.

66. The composition of any one of claims 60-65, wherein the polynucleotide is a messenger RNA (mRNA).

67. The composition of claim 66, wherein the mRNA encodes an antigen or a therapeutic protein.

68. The composition of claim 67, wherein the antigen is an antigen from a pathogen or a cancer-associated antigen.

69. The composition of any one of claims 60-68, wherein the saccharide excipient is mannitol, erythritol or trehalose.

70. The composition of any one of claims 60-68, wherein the saccharide excipient comprises a polyol.

71. The composition of claim 70, wherein the polyol is mannitol.

72. The composition of claim 70, wherein the polyol is erythritol.

73. The composition of any one of claims 60-68, wherein the saccharide excipient comprises an oligosaccharide.

74. The composition of claim 73, wherein the oligosaccharide is trehalose or lactose.

75. The composition of any one of claims 60-74, wherein the lipid nanoparticle comprises an ionizable lipid or a pharmaceutically acceptable salt, solvate, tautomer, stereoisomer, or isotopically labeled derivative thereof, one or more of a PEG-lipid, a phospholipid, a charged lipid, and substituted or unsubstituted cholesterol.

76. The composition of claim 75, wherein the ionizable lipid is CKK-E12 (MD-1), C12-200, Dlin-MC3-DMA (MC3), a compound of any one of claims 1-25, 58, and 59, or a pharmaceutically acceptable salt, solvate, tautomer, stereoisomer, or isotopically labeled derivative thereof, or a lipid molecule that is neutral at physiological pH, but will be protonated at a lower pH.

77. The composition of claim 76, wherein the ionizable lipid is CKK-E12 (MD-1), or a pharmaceutically acceptable salt, solvate, tautomer, stereoisomer, or isotopically labeled derivative thereof.

78. The composition of claim 76, wherein the ionizable lipid is a compound of any one of claims 1-25, 58, and 59, or a pharmaceutically acceptable salt, solvate, tautomer, stereoisomer, or isotopically labeled derivative thereof.

79. The composition of claim 78, wherein the ionizable lipid is RCB-02-76-3 or RCB-01-223-3, or a pharmaceutically acceptable salt, solvate, tautomer, stereoisomer, or isotopically labeled derivative thereof.

80. The composition of any one of claims 75-79, wherein the charged lipid is a cationic lipid.

81. The composition of claim 80, wherein the cationic lipid is DOTAP, DDA, or a combination thereof.

82. The composition of claim 75, wherein the phospholipid comprises 1,2-dioleoyl-sn-glycero-3-phosphoethanolamine (DOPE), 1,2-distearoyl-sn-glycero-3-phosphocholine (DSPC), or a combination thereof.

83. The composition of claim 80, wherein the charged lipid comprises DOTAP.

84. The composition of any one of claims 60-83, wherein the lipid nanoparticle further comprises a lipid conjugated to a solubilizing group.

85. The composition of claim 84, wherein the solubilizing group is a polymer of polyethylene glycol (PEG).

86. The composition of any one of claims 75-85, wherein the cholesterol is unsubstituted cholesterol.

87. The composition of any one of claims 60-86, wherein the composition is a pharmaceutical composition comprising at least one pharmaceutically acceptable excipient.

88. The pharmaceutical composition of claim 87, wherein the pharmaceutical composition is a vaccine.

89. The composition of any one of claims 60-88, wherein the composition is formulated for endotracheal, mucosal, intranasal, inhalation or pulmonary delivery.

90. The composition of any one of claims 60-89, wherein at least some of the saccharide excipient in the composition is encapsulated by the lipid nanoparticles.

91. The composition of any one of claims 60-60, wherein the composition is spray freeze dried.

92. Use of the composition according to any one of claims 60-91 for the preparation of a medicament for the prophylaxis, treatment and/or amelioration of a disorder or a disease.

93. A method of treating or preventing a disorder or a disease by administering to a subject in need thereof a therapeutically effective amount of the composition according to any one of claims 60-91.

94. The use of claim 92 or the method of claim 93, wherein the disorder or disease is an allergic disease, an autoimmune disease, an infectious disease or a cancer.

95. The use or the method of claim 94, wherein the infectious disease is a viral, bacterial or protozoological infectious disease.

96. The use of claim 92 or the method of claim 93, wherein the disorder or disease is a lung disease, a cardiovascular disease, or a neuronal disease.

97. The use or the method of claim 96, wherein the disorder or disease is a lung disease.

98. The use or the method of claim 97, wherein the lung disease is asthma, chronic obstructive pulmonary disease (COPD), cystic fibrosis (CF), lymphangioleiomyomatosis (LAM) or pulmonary fibrosis.

99. A method of preparing a dry powder composition, the method comprising:
a) providing an aqueous mixture comprising a polynucleotide, a lipid nanoparticle, and a saccharide excipient, wherein the polynucleotide is encapsulated by the lipid nanoparticles; and
b) spray freeze drying the mixture to form microparticles, wherein at least about 50% of the microparticles in the composition have an aerodynamic size of about 1 micron to about 5 microns, a physical diameter of about 3 microns to about 10 microns, or a combination thereof,
thereby preparing the dry powder composition.

100. The method of claim 99, wherein at least about 70% of the microparticles in the composition have an aerodynamic size of about 1 micron to about 5 microns.

101. The method of claim 99 or 100, wherein at least about 70% of the microparticles in the composition have a physical diameter of between about 3 microns and about 10 microns.

102. The method of any one of claims 99-101, wherein the polynucleotide is at least about 30 nucleotides in length.

103. The method of claim 102, wherein the polynucleotide is at least about 1,000 nucleotides in length.

104. The method of any one of claims 99-103, wherein the polynucleotide is a messenger RNA (mRNA).

105. The method of claim 104, wherein the mRNA encodes an antigen.

106. The method of claim 105, wherein the antigen is an antigen from a pathogen or a cancer-associated antigen.

107. The method of any one of claims 99-106, wherein the aqueous mixture comprises about 1 μg/ml to about 75 μg/ml of the polynucleotide.

108. The method of claim 107, wherein the aqueous mixture comprises about 10 μg/ml to about 50 μg/ml of the polynucleotide.

109. The method of claim 108, wherein the aqueous mixture comprises about 20 μg/ml to about 30 μg/ml of the polynucleotide.

110. The method of any one of claims 99-109, wherein the aqueous mixture comprises about 0.2 mg/ml to about 0.6 mg/ml total lipid mass.

111. The method of claim 110, wherein the aqueous mixture comprises about 0.4 mg/mL to about 0.5 mg/ml total lipid mass.

112. The method of any one of claims 99-111, wherein the saccharide excipient is mannitol, erythritol, or trehalose.

113. The method of any one of claims 99-111, wherein the saccharide excipient comprises a polyol.

114. The method of claim 113, wherein the polyol is mannitol.

115. The method of claim 113, wherein the polyol is erythritol.

116. The method of any one of claims 99-111, wherein the saccharide excipient comprises an oligosaccharide.

117. The method of claim 116, wherein the oligosaccharide is trehalose or lactose.

118. The method of any one of claims 99-117, wherein the lipid nanoparticle comprises about 2% to about 20% of the saccharide excipient.

119. The method of claim 118, wherein the lipid nanoparticle comprises about 2% to about 4% of the saccharide excipient.

120. The method of any one of claims 99-119, wherein the aqueous mixture comprises about 0.25% to about 2.5% of the saccharide excipient.

121. The method of claim 120, wherein the aqueous mixture comprises about 0.25% to about 0.5% of the saccharide excipient.

122. The method of any one of claims 99-121, wherein the weight ratio of the saccharide excipient to the polynucleotide is about 50:1 to about 1000:1.

123. The method of claim 122, wherein the weight ratio of the saccharide excipient to polynucleotide is about 50:1 to about 200:1.

124. The method of claim 123, wherein the weight ratio of the saccharide excipient to polynucleotide is about 100:1.

125. The method of any one of claims 99-124, wherein the lipid nanoparticle comprises an ionizable lipid or a pharmaceutically acceptable salt, solvate, tautomer, stereoisomer, or isotopically labeled derivative thereof, and one or more of a substituted or unsubstituted cholesterol, a charged lipid, and a phospholipid.

126. The method of claim 125, wherein the ionizable lipid is CKK-E12 (MD-1), C12-200, Dlin-MC3-DMA (MC3), or a compound of any one of claims 1-25, 58, and 59, or a pharmaceutically acceptable salt, solvate, tautomer, stereoisomer, or isotopically labeled derivative thereof, or a lipid molecule that is neutral at physiological pH, but will be protonated at a lower pH.

127. The method of claim 126, wherein the ionizable lipid is CKK-E12 (MD-1), or a pharmaceutically acceptable salt, solvate, tautomer, stereoisomer, or isotopically labeled derivative thereof.

128. The method of claim 126, wherein the ionizable lipid is a compound of any one of claims 1-25, 58, and 59, or a pharmaceutically acceptable salt, solvate, tautomer, stereoisomer, or isotopically labeled derivative thereof.

129. The method of claim 128, wherein the compound is RCB-02-76-3 or RCB-01-223-3, or a pharmaceutically acceptable salt, solvate, tautomer, stereoisomer, or isotopically labeled derivative thereof.

130. The method of any one of claims 125-129, wherein the charged lipid is a cationic lipid.

131. The method of claim 130, wherein the cationic lipid is 1,2-dioleoyl-3-trimethylammonium-propane (DOTAP), dimethyldioctadecylammonium (DDA), or a combination thereof.

132. The method of any one of claims 125-131, wherein the phospholipid is 1,2-dioleoyl-sn-glycero-3-phosphoethanolamine (DOPE) or 1,2-distearoyl-sn-glycero-3-phosphocholine (DSPC), or a combination thereof.

133. The method of claim 132, wherein the charged lipid is DOTAP.

134. The method of any one of claims 99-133, wherein the lipid nanoparticle further comprises a lipid conjugated to a solubilizing group.

135. The method of claim 134, wherein the solubilizing group is a polymer of polyethylene glycol (PEG).

136. The method of any one of claims 125-135, wherein the cholesterol is unsubstituted cholesterol.
